SEQUENCE LISTING (492 sequences)

SEQ ID NO: 1 - protein, 323 aa, synthetic construct
  REGION 1..323: note = Human TROP2
MARGPGLAPPPLRLPLLLLVLAAVTGHTAAQDNCTCPTNKMTVCSPDGPGGRCQCRALGSGMAVDCSTLTSKCLLLKARMSAPKNARTLVRPSEHALVDNDGLYDPDCDPEGRFKARQCNQTSVCWCVNSVGVRRTDKGDLSLRCDELVRTHHILIDLRHRPTAGAFNHSDLDAELRRLFRERYRLHPKFVAAVHYEQPTIQIELRQNTSQKAAGDVDIGDAAYYFERDIKGESLFQGRGGLDLRVRGEPLQVERTLIYYLDEIPPKFSMKRLTAGLIAVIVVVVVALVAGMAVLVITNRRKSGKYKKVEIKELGELRKEPSL

SEQ ID NO: 2 - protein, 297 aa, synthetic construct
  REGION 1..297: note = Mature Human TROP2
HTAAQDNCTCPTNKMTVCSPDGPGGRCQCRALGSGMAVDCSTLTSKCLLLKARMSAPKNARTLVRPSEHALVDNDGLYDPDCDPEGRFKARQCNQTSVCWCVNSVGVRRTDKGDLSLRCDELVRTHHILIDLRHRPTAGAFNHSDLDAELRRLFRERYRLHPKFVAAVHYEQPTIQIELRQNTSQKAAGDVDIGDAAYYFERDIKGESLFQGRGGLDLRVRGEPLQVERTLIYYLDEIPPKFSMKRLTAGLIAVIVVVVVALVAGMAVLVITNRRKSGKYKKVEIKELGELRKEPSL

SEQ ID NO: 3 - protein, 248 aa, synthetic construct
  REGION 1..248: note = Extracellular domain of TROP2
HTAAQDNCTCPTNKMTVCSPDGPGGRCQCRALGSGMAVDCSTLTSKCLLLKARMSAPKNARTLVRPSEHALVDNDGLYDPDCDPEGRFKARQCNQTSVCWCVNSVGVRRTDKGDLSLRCDELVRTHHILIDLRHRPTAGAFNHSDLDAELRRLFRERYRLHPKFVAAVHYEQPTIQIELRQNTSQKAAGDVDIGDAAYYFERDIKGESLFQGRGGLDLRVRGEPLQVERTLIYYLDEIPPKFSMKRLT

SEQ ID NO: 4 - protein, 121 aa, synthetic construct
  REGION 1..121: note = VH domain of Sacituzumab IgG
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSS

SEQ ID NO: 5 - protein, 107 aa, synthetic construct
  REGION 1..107: note = VL domain of Sacituzumab IgG
DIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIK

SEQ ID NO: 6 - protein, 10 aa, synthetic construct
  REGION 1..10: note = Heavy Chain CDR1 of Sacituzumab IgG
GYTFTNYGMN

SEQ ID NO: 7 - protein, 17 aa, synthetic construct
  REGION 1..17: note = Heavy Chain CDR2 of Sacituzumab IgG
WINTYTGEPTYTDDFKG

SEQ ID NO: 8 - protein, 14 aa, synthetic construct
  REGION 1..14: note = Heavy Chain CDR3 of Sacituzumab IgG
ARGGFGSSYWYFDV

SEQ ID NO: 9 - protein, 11 aa, synthetic construct
  REGION 1..11: note = Light Chain CDR1 of Sacituzumab IgG
KASQDVSIAVA

SEQ ID NO: 10 - protein, 7 aa, synthetic construct
  REGION 1..7: note = Light Chain CDR2 of Sacituzumab IgG
SASYRYT

SEQ ID NO: 11 - protein, 9 aa, synthetic construct
  REGION 1..9: note = Light Chain CDR3 of Sacituzumab IgG
QQHYITPLT

SEQ ID NO: 12 - protein, 121 aa, synthetic construct
  REGION 1..121: note = VH domain of Sacituzumab IgG histidine scanning variant #1
QVQLQQSGSELKKPGASVKVSCKASHYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSS

SEQ ID NO: 13 - protein, 121 aa, synthetic construct
  REGION 1..121: note = VH domain of Sacituzumab IgG histidine scanning variant #2
QVQLQQSGSELKKPGASVKVSCKASGHTFTNYGMNWVKQAPGQGLKWMGWINTYTGEPTYTDDFKGRFAFSLDTSVSTAYLQISSLKADDTAVYFCARGGFGSSYWYFDVWGQGSLVTVSS

SEQ ID NO: 14 - protein, 121 aa, synthetic construct
  REGION 1..121: note = VH domain of Sacituzumab IgG histidine scanning variant #3
QVQLQQSGSELKKPGASVKVSCKASGYHFTNYGMNWVKQAPGQGLKWMGWINTYTGEPTYTDDFKGRFAFSLDTSVSTAYLQISSLKADDTAVYFCARGGFGSSYWYFDVWGQGSLVTVSS

SEQ ID NO: 15 - protein, 121 aa, synthetic construct
  REGION 1..121: note = VH domain of Sacituzumab IgG histidine scanning variant #4
QVQLQQSGSELKKPGASVKVSCKASGYTHTNYGMNWVKQAPGQGLKWMGWINTYTGEPTYTDDFKGRFAFSLDTSVSTAYLQISSLKADDTAVYFCARGGFGSSYWYFDVWGQGSLVTVSS

SEQ ID NO: 16 - protein, 121 aa, synthetic construct
  REGION 1..121: note = VH domain of Sacituzumab IgG histidine scanning variant #5
QVQLQQSGSELKKPGASVKVSCKASGYTFHNYGMNWVKQAPGQGLKWMGWINTYTGEPTYTDDFKGRFAFSLDTSVSTAYLQISSLKADDTAVYFCARGGFGSSYWYFDVWGQGSLVTVSS

SEQ ID NO: 17 - protein, 121 aa, synthetic construct
  REGION 1..121: note = VH domain of Sacituzumab IgG histidine scanning variant #6
QVQLQQSGSELKKPGASVKVSCKASGYTFTHYGMNWVKQAPGQGLKWMGWINTYTGEPTYTDDFKGRFAFSLDTSVSTAYLQISSLKADDTAVYFCARGGFGSSYWYFDVWGQGSLVTVSS

SEQ ID NO: 18 - protein, 121 aa, synthetic construct
  REGION 1..121: note = VH domain of Sacituzumab IgG histidine scanning variant #7
QVQLQQSGSELKKPGASVKVSCKASGYTFTNHGMNWVKQAPGQGLKWMGWINTYTGEPTYTDDFKGRFAFSLDTSVSTAYLQISSLKADDTAVYFCARGGFGSSYWYFDVWGQGSLVTVSS

SEQ ID NO: 19 - protein, 121 aa, synthetic construct
  REGION 1..121: note = VH domain of Sacituzumab IgG histidine scanning variant #8
QVQLQQSGSELKKPGASVKVSCKASGYTFTNYHMNWVKQAPGQGLKWMGWINTYTGEPTYTDDFKGRFAFSLDTSVSTAYLQISSLKADDTAVYFCARGGFGSSYWYFDVWGQGSLVTVSS

SEQ ID NO: 20 - protein, 121 aa, synthetic construct
  REGION 1..121: note = VH domain of Sacituzumab IgG histidine scanning variant #9
QVQLQQSGSELKKPGASVKVSCKASGYTFTNYGHNWVKQAPGQGLKWMGWINTYTGEPTYTDDFKGRFAFSLDTSVSTAYLQISSLKADDTAVYFCARGGFGSSYWYFDVWGQGSLVTVSS

SEQ ID NO: 21 - protein, 121 aa, synthetic construct
  REGION 1..121: note = VH domain of Sacituzumab IgG histidine scanning variant #10
QVQLQQSGSELKKPGASVKVSCKASGYTFTNYGMHWVKQAPGQGLKWMGWINTYTGEPTYTDDFKGRFAFSLDTSVSTAYLQISSLKADDTAVYFCARGGFGSSYWYFDVWGQGSLVTVSS

SEQ ID NO: 22 - protein, 121 aa, synthetic construct
  REGION 1..121: note = VH domain of Sacituzumab IgG histidine scanning variant #11
QVQLQQSGSELKKPGASVKVSCKASGYTFTNYGMNWVKQAPGQGLKWMGHINTYTGEPTYTDDFKGRFAFSLDTSVSTAYLQISSLKADDTAVYFCARGGFGSSYWYFDVWGQGSLVTVSS

SEQ ID NO: 23 - protein, 121 aa, synthetic construct
  REGION 1..121: note = VH domain of Sacituzumab IgG histidine scanning variant #12
QVQLQQSGSELKKPGASVKVSCKASGYTFTNYGMNWVKQAPGQGLKWMGWHNTYTGEPTYTDDFKGRFAFSLDTSVSTAYLQISSLKADDTAVYFCARGGFGSSYWYFDVWGQGSLVTVSS

SEQ ID NO: 24 - protein, 121 aa, synthetic construct
  REGION 1..121: note = VH domain of Sacituzumab IgG histidine scanning variant #13
QVQLQQSGSELKKPGASVKVSCKASGYTFTNYGMNWVKQAPGQGLKWMGWIHTYTGEPTYTDDFKGRFAFSLDTSVSTAYLQISSLKADDTAVYFCARGGFGSSYWYFDVWGQGSLVTVSS

SEQ ID NO: 25 - protein, 121 aa, synthetic construct
  REGION 1..121: note = VH domain of Sacituzumab IgG histidine scanning variant #14
QVQLQQSGSELKKPGASVKVSCKASGYTFTNYGMNWVKQAPGQGLKWMGWINHYTGEPTYTDDFKGRFAFSLDTSVSTAYLQISSLKADDTAVYFCARGGFGSSYWYFDVWGQGSLVTVSS

SEQ ID NO: 26 - protein, 121 aa, synthetic construct
  REGION 1..121: note = VH domain of Sacituzumab IgG histidine scanning variant #15
QVQLQQSGSELKKPGASVKVSCKASGYTFTNYGMNWVKQAPGQGLKWMGWINTHTGEPTYTDDFKGRFAFSLDTSVSTAYLQISSLKADDTAVYFCARGGFGSSYWYFDVWGQGSLVTVSS

SEQ ID NO: 27 - protein, 121 aa, synthetic construct
  REGION 1..121: note = VH domain of Sacituzumab IgG histidine scanning variant #16
QVQLQQSGSELKKPGASVKVSCKASGYTFTNYGMNWVKQAPGQGLKWMGWINTYHGEPTYTDDFKGRFAFSLDTSVSTAYLQISSLKADDTAVYFCARGGFGSSYWYFDVWGQGSLVTVSS

SEQ ID NO: 28 - protein, 121 aa, synthetic construct
  REGION 1..121: note = VH domain of Sacituzumab IgG histidine scanning variant #17
QVQLQQSGSELKKPGASVKVSCKASGYTFTNYGMNWVKQAPGQGLKWMGWINTYTHEPTYTDDFKGRFAFSLDTSVSTAYLQISSLKADDTAVYFCARGGFGSSYWYFDVWGQGSLVTVSS

SEQ ID NO: 29 - protein, 121 aa, synthetic construct
  REGION 1..121: note = VH domain of Sacituzumab IgG histidine scanning variant #18
QVQLQQSGSELKKPGASVKVSCKASGYTFTNYGMNWVKQAPGQGLKWMGWINTYTGHPTYTDDFKGRFAFSLDTSVSTAYLQISSLKADDTAVYFCARGGFGSSYWYFDVWGQGSLVTVSS

SEQ ID NO: 30 - protein, 121 aa, synthetic construct
  REGION 1..121: note = VH domain of Sacituzumab IgG histidine scanning variant #19
QVQLQQSGSELKKPGASVKVSCKASGYTFTNYGMNWVKQAPGQGLKWMGWINTYTGEHTYTDDFKGRFAFSLDTSVSTAYLQISSLKADDTAVYFCARGGFGSSYWYFDVWGQGSLVTVSS

SEQ ID NO: 31 - protein, 121 aa, synthetic construct
  REGION 1..121: note = VH domain of Sacituzumab IgG histidine scanning variant #20
QVQLQQSGSELKKPGASVKVSCKASGYTFTNYGMNWVKQAPGQGLKWMGWINTYTGEPHYTDDFKGRFAFSLDTSVSTAYLQISSLKADDTAVYFCARGGFGSSYWYFDVWGQGSLVTVSS

SEQ ID NO: 32 - protein, 121 aa, synthetic construct
  REGION 1..121: note = VH domain of Sacituzumab IgG histidine scanning variant #21
QVQLQQSGSELKKPGASVKVSCKASGYTFTNYGMNWVKQAPGQGLKWMGWINTYTGEPTHTDDFKGRFAFSLDTSVSTAYLQISSLKADDTAVYFCARGGFGSSYWYFDVWGQGSLVTVSS

SEQ ID NO: 33 - protein, 121 aa, synthetic construct
  REGION 1..121: note = VH domain of Sacituzumab IgG histidine scanning variant #22
QVQLQQSGSELKKPGASVKVSCKASGYTFTNYGMNWVKQAPGQGLKWMGWINTYTGEPTYHDDFKGRFAFSLDTSVSTAYLQISSLKADDTAVYFCARGGFGSSYWYFDVWGQGSLVTVSS

SEQ ID NO: 34 - protein, 121 aa, synthetic construct
  REGION 1..121: note = VH domain of Sacituzumab IgG histidine scanning variant #23
QVQLQQSGSELKKPGASVKVSCKASGYTFTNYGMNWVKQAPGQGLKWMGWINTYTGEPTYTHDFKGRFAFSLDTSVSTAYLQISSLKADDTAVYFCARGGFGSSYWYFDVWGQGSLVTVSS

SEQ ID NO: 35 - protein, 121 aa, synthetic construct
  REGION 1..121: note = VH domain of Sacituzumab IgG histidine scanning variant #24
QVQLQQSGSELKKPGASVKVSCKASGYTFTNYGMNWVKQAPGQGLKWMGWINTYTGEPTYTDHFKGRFAFSLDTSVSTAYLQISSLKADDTAVYFCARGGFGSSYWYFDVWGQGSLVTVSS

SEQ ID NO: 36 - protein, 121 aa, synthetic construct
  REGION 1..121: note = VH domain of Sacituzumab IgG histidine scanning variant #25
QVQLQQSGSELKKPGASVKVSCKASGYTFTNYGMNWVKQAPGQGLKWMGWINTYTGEPTYTDDHKGRFAFSLDTSVSTAYLQISSLKADDTAVYFCARGGFGSSYWYFDVWGQGSLVTVSS

SEQ ID NO: 37 - protein, 121 aa, synthetic construct
  REGION 1..121: note = VH domain of Sacituzumab IgG histidine scanning variant #26
QVQLQQSGSELKKPGASVKVSCKASGYTFTNYGMNWVKQAPGQGLKWMGWINTYTGEPTYTDDFHGRFAFSLDTSVSTAYLQISSLKADDTAVYFCARGGFGSSYWYFDVWGQGSLVTVSS

SEQ ID NO: 38 - protein, 121 aa, synthetic construct
  REGION 1..121: note = VH domain of Sacituzumab IgG histidine scanning variant #27
QVQLQQSGSELKKPGASVKVSCKASGYTFTNYGMNWVKQAPGQGLKWMGWINTYTGEPTYTDDFKHRFAFSLDTSVSTAYLQISSLKADDTAVYFCARGGFGSSYWYFDVWGQGSLVTVSS

SEQ ID NO: 39 - protein, 121 aa, synthetic construct
  REGION 1..121: note = VH domain of Sacituzumab IgG histidine scanning variant #28
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCHRGGFGSSYWYFDVWGQGSLVTVSS

SEQ ID NO: 40 - protein, 121 aa, synthetic construct
  REGION 1..121: note = VH domain of Sacituzumab IgG histidine scanning variant #29
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCAHGGFGSSYWYFDVWGQGSLVTVSS

SEQ ID NO: 41 - protein, 121 aa, synthetic construct
  REGION 1..121: note = VH domain of Sacituzumab IgG histidine scanning variant #30
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARHGFGSSYWYFDVWGQGSLVTVSS

SEQ ID NO: 42 - protein, 121 aa, synthetic construct
  REGION 1..121: note = VH domain of Sacituzumab IgG histidine scanning variant #31
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGHFGSSYWYFDVWGQGSLVTVSS

SEQ ID NO: 43 - protein, 121 aa, synthetic construct
  REGION 1..121: note = VH domain of Sacituzumab IgG histidine scanning variant #32
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGHGSSYWYFDVWGQGSLVTVSS

SEQ ID NO: 44 - protein, 121 aa, synthetic construct
  REGION 1..121: note = VH domain of Sacituzumab IgG histidine scanning variant #33
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFHSSYWYFDVWGQGSLVTVSS

SEQ ID NO: 45 - protein, 121 aa, synthetic construct
  REGION 1..121: note = VH domain of Sacituzumab IgG histidine scanning variant #34
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGHSYWYFDVWGQGSLVTVSS

SEQ ID NO: 46 - protein, 121 aa, synthetic construct
  REGION 1..121: note = VH domain of Sacituzumab IgG histidine scanning variant #35
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSHYWYFDVWGQGSLVTVSS

SEQ ID NO: 47 - protein, 121 aa, synthetic construct
  REGION 1..121: note = VH domain of Sacituzumab IgG histidine scanning variant #36
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSHWYFDVWGQGSLVTVSS

SEQ ID NO: 48 - protein, 121 aa, synthetic construct
  REGION 1..121: note = VH domain of Sacituzumab IgG histidine scanning variant #37
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYHYFDVWGQGSLVTVSS

SEQ ID NO: 49 - protein, 121 aa, synthetic construct
  REGION 1..121: note = VH domain of Sacituzumab IgG histidine scanning variant #38
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWHFDVWGQGSLVTVSS

SEQ ID NO: 50 - protein, 121 aa, synthetic construct
  REGION 1..121: note = VH domain of Sacituzumab IgG histidine scanning variant #39
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYHDVWGQGSLVTVSS

SEQ ID NO: 51 - protein, 121 aa, synthetic construct
  REGION 1..121: note = VH domain of Sacituzumab IgG histidine scanning variant #40
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFHVWGQGSLVTVSS

SEQ ID NO: 52 - protein, 121 aa, synthetic construct
  REGION 1..121: note = VH domain of Sacituzumab IgG histidine scanning variant #41
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDHWGQGSLVTVSS

SEQ ID NO: 53 - protein, 107 aa, synthetic construct
  REGION 1..107: note = VL domain of Sacituzumab IgG histidine scanning variant #1
DIQLTQSPSSLSASVGDRVSITCHASQDVSIAVAWYQQKPGKAPKLLIYSASYRYTGVPDRFSGSGSGTDFTLTISSLQPEDFAVYYCQQHYITPLTFGAGTKVEIK

SEQ ID NO: 54 - protein, 107 aa, synthetic construct
  REGION 1..107: note = VL domain of Sacituzumab IgG histidine scanning variant #2
DIQLTQSPSSLSASVGDRVSITCKHSQDVSIAVAWYQQKPGKAPKLLIYSASYRYTGVPDRFSGSGSGTDFTLTISSLQPEDFAVYYCQQHYITPLTFGAGTKVEIK

SEQ ID NO: 55 - protein, 107 aa, synthetic construct
  REGION 1..107: note = VL domain of Sacituzumab IgG histidine scanning variant #3
DIQLTQSPSSLSASVGDRVSITCKAHQDVSIAVAWYQQKPGKAPKLLIYSASYRYTGVPDRFSGSGSGTDFTLTISSLQPEDFAVYYCQQHYITPLTFGAGTKVEIK

SEQ ID NO: 56 - protein, 107 aa, synthetic construct
  REGION 1..107: note = VL domain of Sacituzumab IgG histidine scanning variant #4
DIQLTQSPSSLSASVGDRVSITCKASHDVSIAVAWYQQKPGKAPKLLIYSASYRYTGVPDRFSGSGSGTDFTLTISSLQPEDFAVYYCQQHYITPLTFGAGTKVEIK

SEQ ID NO: 57 - protein, 107 aa, synthetic construct
  REGION 1..107: note = VL domain of Sacituzumab IgG histidine scanning variant #5
DIQLTQSPSSLSASVGDRVSITCKASQHVSIAVAWYQQKPGKAPKLLIYSASYRYTGVPDRFSGSGSGTDFTLTISSLQPEDFAVYYCQQHYITPLTFGAGTKVEIK

SEQ ID NO: 58 - protein, 107 aa, synthetic construct
  REGION 1..107: note = VL domain of Sacituzumab IgG histidine scanning variant #6
DIQLTQSPSSLSASVGDRVSITCKASQDHSIAVAWYQQKPGKAPKLLIYSASYRYTGVPDRFSGSGSGTDFTLTISSLQPEDFAVYYCQQHYITPLTFGAGTKVEIK

SEQ ID NO: 59 - protein, 107 aa, synthetic construct
  REGION 1..107: note = VL domain of Sacituzumab IgG histidine scanning variant #7
DIQLTQSPSSLSASVGDRVSITCKASQDVHIAVAWYQQKPGKAPKLLIYSASYRYTGVPDRFSGSGSGTDFTLTISSLQPEDFAVYYCQQHYITPLTFGAGTKVEIK

SEQ ID NO: 60 - protein, 107 aa, synthetic construct
  REGION 1..107: note = VL domain of Sacituzumab IgG histidine scanning variant #8
DIQLTQSPSSLSASVGDRVSITCKASQDVSHAVAWYQQKPGKAPKLLIYSASYRYTGVPDRFSGSGSGTDFTLTISSLQPEDFAVYYCQQHYITPLTFGAGTKVEIK

SEQ ID NO: 61 - protein, 107 aa, synthetic construct
  REGION 1..107: note = VL domain of Sacituzumab IgG histidine scanning variant #9
DIQLTQSPSSLSASVGDRVSITCKASQDVSIHVAWYQQKPGKAPKLLIYSASYRYTGVPDRFSGSGSGTDFTLTISSLQPEDFAVYYCQQHYITPLTFGAGTKVEIK

SEQ ID NO: 62 - protein, 107 aa, synthetic construct
  REGION 1..107: note = VL domain of Sacituzumab IgG histidine scanning variant #10
DIQLTQSPSSLSASVGDRVSITCKASQDVSIAHAWYQQKPGKAPKLLIYSASYRYTGVPDRFSGSGSGTDFTLTISSLQPEDFAVYYCQQHYITPLTFGAGTKVEIK

SEQ ID NO: 63 - protein, 107 aa, synthetic construct
  REGION 1..107: note = VL domain of Sacituzumab IgG histidine scanning variant #11
DIQLTQSPSSLSASVGDRVSITCKASQDVSIAVHWYQQKPGKAPKLLIYSASYRYTGVPDRFSGSGSGTDFTLTISSLQPEDFAVYYCQQHYITPLTFGAGTKVEIK

SEQ ID NO: 64 - protein, 107 aa, synthetic construct
  REGION 1..107: note = VL domain of Sacituzumab IgG histidine scanning variant #12
DIQLTQSPSSLSASVGDRVSITCKASQDVSIAVAWYQQKPGKAPKLLIYHASYRYTGVPDRFSGSGSGTDFTLTISSLQPEDFAVYYCQQHYITPLTFGAGTKVEIK

SEQ ID NO: 65 - protein, 107 aa, synthetic construct
  REGION 1..107: note = VL domain of Sacituzumab IgG histidine scanning variant #13
DIQLTQSPSSLSASVGDRVSITCKASQDVSIAVAWYQQKPGKAPKLLIYSHSYRYTGVPDRFSGSGSGTDFTLTISSLQPEDFAVYYCQQHYITPLTFGAGTKVEIK

SEQ ID NO: 66 - protein, 107 aa, synthetic construct
  REGION 1..107: note = VL domain of Sacituzumab IgG histidine scanning variant #14
DIQLTQSPSSLSASVGDRVSITCKASQDVSIAVAWYQQKPGKAPKLLIYSAHYRYTGVPDRFSGSGSGTDFTLTISSLQPEDFAVYYCQQHYITPLTFGAGTKVEIK

SEQ ID NO: 67 - protein, 107 aa, synthetic construct
  REGION 1..107: note = VL domain of Sacituzumab IgG histidine scanning variant #15
DIQLTQSPSSLSASVGDRVSITCKASQDVSIAVAWYQQKPGKAPKLLIYSASHRYTGVPDRFSGSGSGTDFTLTISSLQPEDFAVYYCQQHYITPLTFGAGTKVEIK

SEQ ID NO: 68 - protein, 107 aa, synthetic construct
  REGION 1..107: note = VL domain of Sacituzumab IgG histidine scanning variant #16
DIQLTQSPSSLSASVGDRVSITCKASQDVSIAVAWYQQKPGKAPKLLIYSASYHYTGVPDRFSGSGSGTDFTLTISSLQPEDFAVYYCQQHYITPLTFGAGTKVEIK

SEQ ID NO: 69 - protein, 107 aa, synthetic construct
  REGION 1..107: note = VL domain of Sacituzumab IgG histidine scanning variant #17
DIQLTQSPSSLSASVGDRVSITCKASQDVSIAVAWYQQKPGKAPKLLIYSASYRHTGVPDRFSGSGSGTDFTLTISSLQPEDFAVYYCQQHYITPLTFGAGTKVEIK

SEQ ID NO: 70 - protein, 107 aa, synthetic construct
  REGION 1..107: note = VL domain of Sacituzumab IgG histidine scanning variant #18
DIQLTQSPSSLSASVGDRVSITCKASQDVSIAVAWYQQKPGKAPKLLIYSASYRYHGVPDRFSGSGSGTDFTLTISSLQPEDFAVYYCQQHYITPLTFGAGTKVEIK

SEQ ID NO: 71 - protein, 107 aa, synthetic construct
  REGION 1..107: note = VL domain of Sacituzumab IgG histidine scanning variant #19
DIQLTQSPSSLSASVGDRVSITCKASQDVSIAVAWYQQKPGKAPKLLIYSASYRYTGVPDRFSGSGSGTDFTLTISSLQPEDFAVYYCHQHYITPLTFGAGTKVEIK

SEQ ID NO: 72 - protein, 107 aa, synthetic construct
  REGION 1..107: note = VL domain of Sacituzumab IgG histidine scanning variant #20
DIQLTQSPSSLSASVGDRVSITCKASQDVSIAVAWYQQKPGKAPKLLIYSASYRYTGVPDRFSGSGSGTDFTLTISSLQPEDFAVYYCQHHYITPLTFGAGTKVEIK

SEQ ID NO: 73 - protein, 107 aa, synthetic construct
  REGION 1..107: note = VL domain of Sacituzumab IgG histidine scanning variant #21
DIQLTQSPSSLSASVGDRVSITCKASQDVSIAVAWYQQKPGKAPKLLIYSASYRYTGVPDRFSGSGSGTDFTLTISSLQPEDFAVYYCQQAYITPLTFGAGTKVEIK

SEQ ID NO: 74 - protein, 107 aa, synthetic construct
  REGION 1..107: note = VL domain of Sacituzumab IgG histidine scanning variant #22
DIQLTQSPSSLSASVGDRVSITCKASQDVSIAVAWYQQKPGKAPKLLIYSASYRYTGVPDRFSGSGSGTDFTLTISSLQPEDFAVYYCQQHHITPLTFGAGTKVEIK

SEQ ID NO: 75 - protein, 107 aa, synthetic construct
  REGION 1..107: note = VL domain of Sacituzumab IgG histidine scanning variant #23
DIQLTQSPSSLSASVGDRVSITCKASQDVSIAVAWYQQKPGKAPKLLIYSASYRYTGVPDRFSGSGSGTDFTLTISSLQPEDFAVYYCQQHYHTPLTFGAGTKVEIK

SEQ ID NO: 76 - protein, 107 aa, synthetic construct
  REGION 1..107: note = VL domain of Sacituzumab IgG histidine scanning variant #24
DIQLTQSPSSLSASVGDRVSITCKASQDVSIAVAWYQQKPGKAPKLLIYSASYRYTGVPDRFSGSGSGTDFTLTISSLQPEDFAVYYCQQHYIHPLTFGAGTKVEIK

SEQ ID NO: 77 - protein, 107 aa, synthetic construct
  REGION 1..107: note = VL domain of Sacituzumab IgG histidine scanning variant #25
DIQLTQSPSSLSASVGDRVSITCKASQDVSIAVAWYQQKPGKAPKLLIYSASYRYTGVPDRFSGSGSGTDFTLTISSLQPEDFAVYYCQQHYITHLTFGAGTKVEIK

SEQ ID NO: 78 - protein, 107 aa, synthetic construct
  REGION 1..107: note = VL domain of Sacituzumab IgG histidine scanning variant #26
DIQLTQSPSSLSASVGDRVSITCKASQDVSIAVAWYQQKPGKAPKLLIYSASYRYTGVPDRFSGSGSGTDFTLTISSLQPEDFAVYYCQQHYITPHTFGAGTKVEIK

SEQ ID NO: 79 - protein, 107 aa, synthetic construct
  REGION 1..107: note = VL domain of Sacituzumab IgG histidine scanning variant #27
DIQLTQSPSSLSASVGDRVSITCKASQDVSIAVAWYQQKPGKAPKLLIYSASYRYTGVPDRFSGSGSGTDFTLTISSLQPEDFAVYYCQQHYITPLHFGAGTKVEIK

SEQ ID NO: 80 - protein, 121 aa, synthetic construct
  REGION 1..121: note = VH domain of Datopotamab IgG
QVQLVQSGAEVKKPGASVKVSCKASGYTFTTAGMQWVRQAPGQGLEWMGWINTHSGVPKYAEDFKGRVTISADTSTSTAYLQLSSLKSEDTAVYYCARSGFGSSYWYFDVWGQGTLVTVSS

SEQ ID NO: 81 - protein, 107 aa, synthetic construct
  REGION 1..107: note = VL domain of Datopotamab IgG
DIQMTQSPSSLSASVGDRVTITCKASQDVSTAVAWYQQKPGKAPKLLIYSASYRYTGVPSRFSGSGSGTDFTLTISSLQPEDFAVYYCQQHYITPLTFGQGTKLEIK

SEQ ID NO: 82 - protein, 10 aa, synthetic construct
  REGION 1..10: note = Heavy Chain CDR1 of Datopotamab IgG
GYTFTTAGMQ

SEQ ID NO: 83 - protein, 17 aa, synthetic construct
  REGION 1..17: note = Heavy Chain CDR2 of Datopotamab IgG
WINTHSGVPKYAEDFKG

SEQ ID NO: 84 - protein, 14 aa, synthetic construct
  REGION 1..14: note = Heavy Chain CDR3 of Datopotamab IgG
ARSGFGSSYWYFDV

SEQ ID NO: 85 - protein, 11 aa, synthetic construct
  REGION 1..11: note = Light Chain CDR1 of Datopotamab IgG
KASQDVSTAVA

SEQ ID NO: 86 - protein, 7 aa, synthetic construct
  REGION 1..7: note = Light Chain CDR2 of Datopotamab IgG
SASYRYT

SEQ ID NO: 87 - protein, 9 aa, synthetic construct
  REGION 1..9: note = Light Chain CDR3 of Datopotamab IgG
QQHYITPLT

SEQ ID NO: 88 - protein, 121 aa, synthetic construct
  REGION 1..121: note = VH domain of Datopotamab IgG histidine scanning variant #1
QVQLVQSGAEVKKPGASVKVSCKASHYTFTTAGMQWVRQAPGQGLEWMGWINTHSGVPKYAEDFKGRVTISADTSTSTAYLQLSSLKSEDTAVYYCARSGFGSSYWYFDVWGQGTLVTVSS

SEQ ID NO: 89 - protein, 121 aa, synthetic construct
  REGION 1..121: note = VH domain of Datopotamab IgG histidine scanning variant #2
QVQLVQSGAEVKKPGASVKVSCKASGHTFTTAGMQWVRQAPGQGLEWMGWINTHSGVPKYAEDFKGRVTISADTSTSTAYLQLSSLKSEDTAVYYCARSGFGSSYWYFDVWGQGTLVTVSS

SEQ ID NO: 90 - protein, 121 aa, synthetic construct
  REGION 1..121: note = VH domain of Datopotamab IgG histidine scanning variant #3
QVQLVQSGAEVKKPGASVKVSCKASGYHFTTAGMQWVRQAPGQGLEWMGWINTHSGVPKYAEDFKGRVTISADTSTSTAYLQLSSLKSEDTAVYYCARSGFGSSYWYFDVWGQGTLVTVSS

SEQ ID NO: 91 - protein, 121 aa, synthetic construct
  REGION 1..121: note = VH domain of Datopotamab IgG histidine scanning variant #4
QVQLVQSGAEVKKPGASVKVSCKASGYTHTTAGMQWVRQAPGQGLEWMGWINTHSGVPKYAEDFKGRVTISADTSTSTAYLQLSSLKSEDTAVYYCARSGFGSSYWYFDVWGQGTLVTVSS

SEQ ID NO: 92 - protein, 121 aa, synthetic construct
  REGION 1..121: note = VH domain of Datopotamab IgG histidine scanning variant #5
QVQLVQSGAEVKKPGASVKVSCKASGYTFHTAGMQWVRQAPGQGLEWMGWINTHSGVPKYAEDFKGRVTISADTSTSTAYLQLSSLKSEDTAVYYCARSGFGSSYWYFDVWGQGTLVTVSS

SEQ ID NO: 93 - protein, 121 aa, synthetic construct
  REGION 1..121: note = VH domain of Datopotamab IgG histidine scanning variant #6
QVQLVQSGAEVKKPGASVKVSCKASGYTFTHAGMQWVRQAPGQGLEWMGWINTHSGVPKYAEDFKGRVTISADTSTSTAYLQLSSLKSEDTAVYYCARSGFGSSYWYFDVWGQGTLVTVSS

SEQ ID NO: 94 - protein, 121 aa, synthetic construct
  REGION 1..121: note = VH domain of Datopotamab IgG histidine scanning variant #7
QVQLVQSGAEVKKPGASVKVSCKASGYTFTTHGMQWVRQAPGQGLEWMGWINTHSGVPKYAEDFKGRVTISADTSTSTAYLQLSSLKSEDTAVYYCARSGFGSSYWYFDVWGQGTLVTVSS

SEQ ID NO: 95 - protein, 121 aa, synthetic construct
  REGION 1..121: note = VH domain of Datopotamab IgG histidine scanning variant #8
QVQLVQSGAEVKKPGASVKVSCKASGYTFTTAHMQWVRQAPGQGLEWMGWINTHSGVPKYAEDFKGRVTISADTSTSTAYLQLSSLKSEDTAVYYCARSGFGSSYWYFDVWGQGTLVTVSS

SEQ ID NO: 96 - protein, 121 aa, synthetic construct
  REGION 1..121: note = VH domain of Datopotamab IgG histidine scanning variant #9
QVQLVQSGAEVKKPGASVKVSCKASGYTFTTAGHQWVRQAPGQGLEWMGWINTHSGVPKYAEDFKGRVTISADTSTSTAYLQLSSLKSEDTAVYYCARSGFGSSYWYFDVWGQGTLVTVSS

SEQ ID NO: 97 - protein, 121 aa, synthetic construct
  REGION 1..121: note = VH domain of Datopotamab IgG histidine scanning variant #10
QVQLVQSGAEVKKPGASVKVSCKASGYTFTTAGMHWVRQAPGQGLEWMGWINTHSGVPKYAEDFKGRVTISADTSTSTAYLQLSSLKSEDTAVYYCARSGFGSSYWYFDVWGQGTLVTVSS

SEQ ID NO: 98 - protein, 121 aa, synthetic construct
  REGION 1..121: note = VH domain of Datopotamab IgG histidine scanning variant #11
QVQLVQSGAEVKKPGASVKVSCKASGYTFTTAGMQWVRQAPGQGLEWMGHINTHSGVPKYAEDFKGRVTISADTSTSTAYLQLSSLKSEDTAVYYCARSGFGSSYWYFDVWGQGTLVTVSS

SEQ ID NO: 99 - protein, 121 aa, synthetic construct
  REGION 1..121: note = VH domain of Datopotamab IgG histidine scanning variant #12
QVQLVQSGAEVKKPGASVKVSCKASGYTFTTAGMQWVRQAPGQGLEWMGWHNTHSGVPKYAEDFKGRVTISADTSTSTAYLQLSSLKSEDTAVYYCARSGFGSSYWYFDVWGQGTLVTVSS

SEQ ID NO: 100 - protein, 121 aa, synthetic construct
  REGION 1..121: note = VH domain of Datopotamab IgG histidine scanning variant #13
QVQLVQSGAEVKKPGASVKVSCKASGYTFTTAGMQWVRQAPGQGLEWMGWIHTHSGVPKYAEDFKGRVTISADTSTSTAYLQLSSLKSEDTAVYYCARSGFGSSYWYFDVWGQGTLVTVSS

SEQ ID NO: 101 - protein, 121 aa, synthetic construct
  REGION 1..121: note = VH domain of Datopotamab IgG histidine scanning variant #14
QVQLVQSGAEVKKPGASVKVSCKASGYTFTTAGMQWVRQAPGQGLEWMGWINHHSGVPKYAEDFKGRVTISADTSTSTAYLQLSSLKSEDTAVYYCARSGFGSSYWYFDVWGQGTLVTVSS

SEQ ID NO: 102 - protein, 121 aa, synthetic construct
  REGION 1..121: note = VH domain of Datopotamab IgG histidine scanning variant #15
QVQLVQSGAEVKKPGASVKVSCKASGYTFTTAGMQWVRQAPGQGLEWMGWINTASGVPKYAEDFKGRVTISADTSTSTAYLQLSSLKSEDTAVYYCARSGFGSSYWYFDVWGQGTLVTVSS

SEQ ID NO: 103 - protein, 121 aa, synthetic construct
  REGION 1..121: note = VH domain of Datopotamab IgG histidine scanning variant #16
QVQLVQSGAEVKKPGASVKVSCKASGYTFTTAGMQWVRQAPGQGLEWMGWINTHHGVPKYAEDFKGRVTISADTSTSTAYLQLSSLKSEDTAVYYCARSGFGSSYWYFDVWGQGTLVTVSS

SEQ ID NO: 104 - protein, 121 aa, synthetic construct
  REGION 1..121: note = VH domain of Datopotamab IgG histidine scanning variant #17
QVQLVQSGAEVKKPGASVKVSCKASGYTFTTAGMQWVRQAPGQGLEWMGWINTHSHVPKYAEDFKGRVTISADTSTSTAYLQLSSLKSEDTAVYYCARSGFGSSYWYFDVWGQGTLVTVSS

SEQ ID NO: 105 - protein, 121 aa, synthetic construct
  REGION 1..121: note = VH domain of Datopotamab IgG histidine scanning variant #18
QVQLVQSGAEVKKPGASVKVSCKASGYTFTTAGMQWVRQAPGQGLEWMGWINTHSGHPKYAEDFKGRVTISADTSTSTAYLQLSSLKSEDTAVYYCARSGFGSSYWYFDVWGQGTLVTVSS

SEQ ID NO: 106 - protein, 121 aa, synthetic construct
  REGION 1..121: note = VH domain of Datopotamab IgG histidine scanning variant #19
QVQLVQSGAEVKKPGASVKVSCKASGYTFTTAGMQWVRQAPGQGLEWMGWINTHSGVHKYAEDFKGRVTISADTSTSTAYLQLSSLKSEDTAVYYCARSGFGSSYWYFDVWGQGTLVTVSS

SEQ ID NO: 107 - protein, 121 aa, synthetic construct
  REGION 1..121: note = VH domain of Datopotamab IgG histidine scanning variant #20
QVQLVQSGAEVKKPGASVKVSCKASGYTFTTAGMQWVRQAPGQGLEWMGWINTHSGVPHYAEDFKGRVTISADTSTSTAYLQLSSLKSEDTAVYYCARSGFGSSYWYFDVWGQGTLVTVSS

SEQ ID NO: 108 - protein, 121 aa, synthetic construct
  REGION 1..121: note = VH domain of Datopotamab IgG histidine scanning variant #21
QVQLVQSGAEVKKPGASVKVSCKASGYTFTTAGMQWVRQAPGQGLEWMGWINTHSGVPKHAEDFKGRVTISADTSTSTAYLQLSSLKSEDTAVYYCARSGFGSSYWYFDVWGQGTLVTVSS

SEQ ID NO: 109 - protein, 121 aa, synthetic construct
  REGION 1..121: note = VH domain of Datopotamab IgG histidine scanning variant #22
QVQLVQSGAEVKKPGASVKVSCKASGYTFTTAGMQWVRQAPGQGLEWMGWINTHSGVPKYHEDFKGRVTISADTSTSTAYLQLSSLKSEDTAVYYCARSGFGSSYWYFDVWGQGTLVTVSS

SEQ ID NO: 110 - protein, 121 aa, synthetic construct
  REGION 1..121: note = VH domain of Datopotamab IgG histidine scanning variant #23
QVQLVQSGAEVKKPGASVKVSCKASGYTFTTAGMQWVRQAPGQGLEWMGWINTHSGVPKYAHDFKGRVTISADTSTSTAYLQLSSLKSEDTAVYYCARSGFGSSYWYFDVWGQGTLVTVSS

SEQ ID NO: 111 - protein, 121 aa, synthetic construct
  REGION 1..121: note = VH domain of Datopotamab IgG histidine scanning variant #24
QVQLVQSGAEVKKPGASVKVSCKASGYTFTTAGMQWVRQAPGQGLEWMGWINTHSGVPKYAEHFKGRVTISADTSTSTAYLQLSSLKSEDTAVYYCARSGFGSSYWYFDVWGQGTLVTVSS

SEQ ID NO: 112 - protein, 121 aa, synthetic construct
  REGION 1..121: note = VH domain of Datopotamab IgG histidine scanning variant #25
QVQLVQSGAEVKKPGASVKVSCKASGYTFTTAGMQWVRQAPGQGLEWMGWINTHSGVPKYAEDHKGRVTISADTSTSTAYLQLSSLKSEDTAVYYCARSGFGSSYWYFDVWGQGTLVTVSS

SEQ ID NO: 113 - protein, 121 aa, synthetic construct
  REGION 1..121: note = VH domain of Datopotamab IgG histidine scanning variant #26
QVQLVQSGAEVKKPGASVKVSCKASGYTFTTAGMQWVRQAPGQGLEWMGWINTHSGVPKYAEDFHGRVTISADTSTSTAYLQLSSLKSEDTAVYYCARSGFGSSYWYFDVWGQGTLVTVSS

SEQ ID NO: 114 - protein, 121 aa, synthetic construct
  REGION 1..121: note = VH domain of Datopotamab IgG histidine scanning variant #27
QVQLVQSGAEVKKPGASVKVSCKASGYTFTTAGMQWVRQAPGQGLEWMGWINTHSGVPKYAEDFKHRVTISADTSTSTAYLQLSSLKSEDTAVYYCARSGFGSSYWYFDVWGQGTLVTVSS

SEQ ID NO: 115 - protein, 121 aa, synthetic construct
  REGION 1..121: note = VH domain of Datopotamab IgG histidine scanning variant #28
QVQLVQSGAEVKKPGASVKVSCKASGYTFTTAGMQWVRQAPGQGLEWMGWINTHSGVPKYAEDFKGRVTISADTSTSTAYLQLSSLKSEDTAVYYCHRSGFGSSYWYFDVWGQGTLVTVSS

SEQ ID NO: 116 - protein, 121 aa, synthetic construct
  REGION 1..121: note = VH domain of Datopotamab IgG histidine scanning variant #29
QVQLVQSGAEVKKPGASVKVSCKASGYTFTTAGMQWVRQAPGQGLEWMGWINTHSGVPKYAEDFKGRVTISADTSTSTAYLQLSSLKSEDTAVYYCAHSGFGSSYWYFDVWGQGTLVTVSS

SEQ ID NO: 117 - protein, 121 aa, synthetic construct
  REGION 1..121: note = VH domain of Datopotamab IgG histidine scanning variant #30
QVQLVQSGAEVKKPGASVKVSCKASGYTFTTAGMQWVRQAPGQGLEWMGWINTHSGVPKYAEDFKGRVTISADTSTSTAYLQLSSLKSEDTAVYYCARHGFGSSYWYFDVWGQGTLVTVSS

SEQ ID NO: 118 - protein, 121 aa, synthetic construct
  REGION 1..121: note = VH domain of Datopotamab IgG histidine scanning variant #31
QVQLVQSGAEVKKPGASVKVSCKASGYTFTTAGMQWVRQAPGQGLEWMGWINTHSGVPKYAEDFKGRVTISADTSTSTAYLQLSSLKSEDTAVYYCARSHFGSSYWYFDVWGQGTLVTVSS

SEQ ID NO: 119 - protein, 121 aa, synthetic construct
  REGION 1..121: note = VH domain of Datopotamab IgG histidine scanning variant #32
QVQLVQSGAEVKKPGASVKVSCKASGYTFTTAGMQWVRQAPGQGLEWMGWINTHSGVPKYAEDFKGRVTISADTSTSTAYLQLSSLKSEDTAVYYCARSGHGSSYWYFDVWGQGTLVTVSS

SEQ ID NO: 120 - protein, 121 aa, synthetic construct
  REGION 1..121: note = VH domain of Datopotamab IgG histidine scanning variant #33
QVQLVQSGAEVKKPGASVKVSCKASGYTFTTAGMQWVRQAPGQGLEWMGWINTHSGVPKYAEDFKGRVTISADTSTSTAYLQLSSLKSEDTAVYYCARSGFHSSYWYFDVWGQGTLVTVSS

SEQ ID NO: 121 - protein, 121 aa, synthetic construct
  REGION 1..121: note = VH domain of Datopotamab IgG histidine scanning variant #34
QVQLVQSGAEVKKPGASVKVSCKASGYTFTTAGMQWVRQAPGQGLEWMGWINTHSGVPKYAEDFKGRVTISADTSTSTAYLQLSSLKSEDTAVYYCARSGFGHSYWYFDVWGQGTLVTVSS

SEQ ID NO: 122 - protein, 121 aa, synthetic construct
  REGION 1..121: note = VH domain of Datopotamab IgG histidine scanning variant #35
QVQLVQSGAEVKKPGASVKVSCKASGYTFTTAGMQWVRQAPGQGLEWMGWINTHSGVPKYAEDFKGRVTISADTSTSTAYLQLSSLKSEDTAVYYCARSGFGSHYWYFDVWGQGTLVTVSS

SEQ ID NO: 123 - protein, 121 aa, synthetic construct
  REGION 1..121: note = VH domain of Datopotamab IgG histidine scanning variant #36
QVQLVQSGAEVKKPGASVKVSCKASGYTFTTAGMQWVRQAPGQGLEWMGWINTHSGVPKYAEDFKGRVTISADTSTSTAYLQLSSLKSEDTAVYYCARSGFGSSHWYFDVWGQGTLVTVSS

SEQ ID NO: 124 - protein, 121 aa, synthetic construct
  REGION 1..121: note = VH domain of Datopotamab IgG histidine scanning variant #37
QVQLVQSGAEVKKPGASVKVSCKASGYTFTTAGMQWVRQAPGQGLEWMGWINTHSGVPKYAEDFKGRVTISADTSTSTAYLQLSSLKSEDTAVYYCARSGFGSSYHYFDVWGQGTLVTVSS

SEQ ID NO: 125 - protein, 121 aa, synthetic construct
  REGION 1..121: note = VH domain of Datopotamab IgG histidine scanning variant #38
QVQLVQSGAEVKKPGASVKVSCKASGYTFTTAGMQWVRQAPGQGLEWMGWINTHSGVPKYAEDFKGRVTISADTSTSTAYLQLSSLKSEDTAVYYCARSGFGSSYWHFDVWGQGTLVTVSS

SEQ ID NO: 126 - protein, 121 aa, synthetic construct
  REGION 1..121: note = VH domain of Datopotamab IgG histidine scanning variant #39
QVQLVQSGAEVKKPGASVKVSCKASGYTFTTAGMQWVRQAPGQGLEWMGWINTHSGVPKYAEDFKGRVTISADTSTSTAYLQLSSLKSEDTAVYYCARSGFGSSYWYHDVWGQGTLVTVSS

SEQ ID NO: 127 - protein, 121 aa, synthetic construct
  REGION 1..121: note = VH domain of Datopotamab IgG histidine scanning variant #40
QVQLVQSGAEVKKPGASVKVSCKASGYTFTTAGMQWVRQAPGQGLEWMGWINTHSGVPKYAEDFKGRVTISADTSTSTAYLQLSSLKSEDTAVYYCARSGFGSSYWYFHVWGQGTLVTVSS

SEQ ID NO: 128 - protein, 121 aa, synthetic construct
  REGION 1..121: note = VH domain of Datopotamab IgG histidine scanning variant #41
QVQLVQSGAEVKKPGASVKVSCKASGYTFTTAGMQWVRQAPGQGLEWMGWINTHSGVPKYAEDFKGRVTISADTSTSTAYLQLSSLKSEDTAVYYCARSGFGSSYWYFDHWGQGTLVTVSS

SEQ ID NO: 129 - protein, 107 aa, synthetic construct
  REGION 1..107: note = VL domain of Datopotamab IgG histidine scanning variant #1
DIQMTQSPSSLSASVGDRVTITCHASQDVSTAVAWYQQKPGKAPKLLIYSASYRYTGVPSRFSGSGSGTDFTLTISSLQPEDFAVYYCQQHYITPLTFGQGTKLEIK

SEQ ID NO: 130 - protein, 107 aa, synthetic construct
  REGION 1..107: note = VL domain of Datopotamab IgG histidine scanning variant #2
DIQMTQSPSSLSASVGDRVTITCKHSQDVSTAVAWYQQKPGKAPKLLIYSASYRYTGVPSRFSGSGSGTDFTLTISSLQPEDFAVYYCQQHYITPLTFGQGTKLEIK

SEQ ID NO: 131 - protein, 107 aa, synthetic construct
  REGION 1..107: note = VL domain of Datopotamab IgG histidine scanning variant #3
DIQMTQSPSSLSASVGDRVTITCKAHQDVSTAVAWYQQKPGKAPKLLIYSASYRYTGVPSRFSGSGSGTDFTLTISSLQPEDFAVYYCQQHYITPLTFGQGTKLEIK

SEQ ID NO: 132 - protein, 107 aa, synthetic construct
  REGION 1..107: note = VL domain of Datopotamab IgG histidine scanning variant #4
DIQMTQSPSSLSASVGDRVTITCKASHDVSTAVAWYQQKPGKAPKLLIYSASYRYTGVPSRFSGSGSGTDFTLTISSLQPEDFAVYYCQQHYITPLTFGQGTKLEIK

SEQ ID NO: 133 - protein, 107 aa, synthetic construct
  REGION 1..107: note = VL domain of Datopotamab IgG histidine scanning variant #5
DIQMTQSPSSLSASVGDRVTITCKASQHVSTAVAWYQQKPGKAPKLLIYSASYRYTGVPSRFSGSGSGTDFTLTISSLQPEDFAVYYCQQHYITPLTFGQGTKLEIK

SEQ ID NO: 134 - protein, 107 aa, synthetic construct
  REGION 1..107: note = VL domain of Datopotamab IgG histidine scanning variant #6
DIQMTQSPSSLSASVGDRVTITCKASQDHSTAVAWYQQKPGKAPKLLIYSASYRYTGVPSRFSGSGSGTDFTLTISSLQPEDFAVYYCQQHYITPLTFGQGTKLEIK

SEQ ID NO: 135 - protein, 107 aa, synthetic construct
  REGION 1..107: note = VL domain of Datopotamab IgG histidine scanning variant #7
DIQMTQSPSSLSASVGDRVTITCKASQDVHTAVAWYQQKPGKAPKLLIYSASYRYTGVPSRFSGSGSGTDFTLTISSLQPEDFAVYYCQQHYITPLTFGQGTKLEIK

SEQ ID NO: 136 - protein, 107 aa, synthetic construct
  REGION 1..107: note = VL domain of Datopotamab IgG histidine scanning variant #8
DIQMTQSPSSLSASVGDRVTITCKASQDVSHAVAWYQQKPGKAPKLLIYSASYRYTGVPSRFSGSGSGTDFTLTISSLQPEDFAVYYCQQHYITPLTFGQGTKLEIK

SEQ ID NO: 137 - protein, 107 aa, synthetic construct
  REGION 1..107: note = VL domain of Datopotamab IgG histidine scanning variant #9
DIQMTQSPSSLSASVGDRVTITCKASQDVSTHVAWYQQKPGKAPKLLIYSASYRYTGVPSRFSGSGSGTDFTLTISSLQPEDFAVYYCQQHYITPLTFGQGTKLEIK

SEQ ID NO: 138 - protein, 107 aa, synthetic construct
  REGION 1..107: note = VL domain of Datopotamab IgG histidine scanning variant #10
DIQMTQSPSSLSASVGDRVTITCKASQDVSTAHAWYQQKPGKAPKLLIYSASYRYTGVPSRFSGSGSGTDFTLTISSLQPEDFAVYYCQQHYITPLTFGQGTKLEIK

SEQ ID NO: 139 - protein, 107 aa, synthetic construct
  REGION 1..107: note = VL domain of Datopotamab IgG histidine scanning variant #11
DIQMTQSPSSLSASVGDRVTITCKASQDVSTAVHWYQQKPGKAPKLLIYSASYRYTGVPSRFSGSGSGTDFTLTISSLQPEDFAVYYCQQHYITPLTFGQGTKLEIK

SEQ ID NO: 140 - protein, 107 aa, synthetic construct
  REGION 1..107: note = VL domain of Datopotamab IgG histidine scanning variant #12
DIQMTQSPSSLSASVGDRVTITCKASQDVSTAVAWYQQKPGKAPKLLIYHASYRYTGVPSRFSGSGSGTDFTLTISSLQPEDFAVYYCQQHYITPLTFGQGTKLEIK

SEQ ID NO: 141 - protein, 107 aa, synthetic construct
  REGION 1..107: note = VL domain of Datopotamab IgG histidine scanning variant #13
DIQMTQSPSSLSASVGDRVTITCKASQDVSTAVAWYQQKPGKAPKLLIYSHSYRYTGVPSRFSGSGSGTDFTLTISSLQPEDFAVYYCQQHYITPLTFGQGTKLEIK

SEQ ID NO: 142 - protein, 107 aa, synthetic construct
  REGION 1..107: note = VL domain of Datopotamab IgG histidine scanning variant #14
DIQMTQSPSSLSASVGDRVTITCKASQDVSTAVAWYQQKPGKAPKLLIYSAHYRYTGVPSRFSGSGSGTDFTLTISSLQPEDFAVYYCQQHYITPLTFGQGTKLEIK

SEQ ID NO: 143 - protein, 107 aa, synthetic construct
  REGION 1..107: note = VL domain of Datopotamab IgG histidine scanning variant #15
DIQMTQSPSSLSASVGDRVTITCKASQDVSTAVAWYQQKPGKAPKLLIYSASHRYTGVPSRFSGSGSGTDFTLTISSLQPEDFAVYYCQQHYITPLTFGQGTKLEIK

SEQ ID NO: 144 - protein, 107 aa, synthetic construct
  REGION 1..107: note = VL domain of Datopotamab IgG histidine scanning variant #16
DIQMTQSPSSLSASVGDRVTITCKASQDVSTAVAWYQQKPGKAPKLLIYSASYHYTGVPSRFSGSGSGTDFTLTISSLQPEDFAVYYCQQHYITPLTFGQGTKLEIK

SEQ ID NO: 145 - protein, 107 aa, synthetic construct
  REGION 1..107: note = VL domain of Datopotamab IgG histidine scanning variant #17
DIQMTQSPSSLSASVGDRVTITCKASQDVSTAVAWYQQKPGKAPKLLIYSASYRHTGVPSRFSGSGSGTDFTLTISSLQPEDFAVYYCQQHYITPLTFGQGTKLEIK

SEQ ID NO: 146 - protein, 107 aa, synthetic construct
  REGION 1..107: note = VL domain of Datopotamab IgG histidine scanning variant #18
DIQMTQSPSSLSASVGDRVTITCKASQDVSTAVAWYQQKPGKAPKLLIYSASYRYHGVPSRFSGSGSGTDFTLTISSLQPEDFAVYYCQQHYITPLTFGQGTKLEIK

SEQ ID NO: 147 - protein, 107 aa, synthetic construct
  REGION 1..107: note = VL domain of Datopotamab IgG histidine scanning variant #19
DIQMTQSPSSLSASVGDRVTITCKASQDVSTAVAWYQQKPGKAPKLLIYSASYRYTGVPSRFSGSGSGTDFTLTISSLQPEDFAVYYCHQHYITPLTFGQGTKLEIK

SEQ ID NO: 148 - protein, 107 aa, synthetic construct
  REGION 1..107: note = VL domain of Datopotamab IgG histidine scanning variant #20
DIQMTQSPSSLSASVGDRVTITCKASQDVSTAVAWYQQKPGKAPKLLIYSASYRYTGVPSRFSGSGSGTDFTLTISSLQPEDFAVYYCQHHYITPLTFGQGTKLEIK

SEQ ID NO: 149 - protein, 107 aa, synthetic construct
  REGION 1..107: note = VL domain of Datopotamab IgG histidine scanning variant #21
DIQMTQSPSSLSASVGDRVTITCKASQDVSTAVAWYQQKPGKAPKLLIYSASYRYTGVPSRFSGSGSGTDFTLTISSLQPEDFAVYYCQQAYITPLTFGQGTKLEIK

SEQ ID NO: 150 - protein, 107 aa, synthetic construct
  REGION 1..107: note = VL domain of Datopotamab IgG histidine scanning variant #22
DIQMTQSPSSLSASVGDRVTITCKASQDVSTAVAWYQQKPGKAPKLLIYSASYRYTGVPSRFSGSGSGTDFTLTISSLQPEDFAVYYCQQHHITPLTFGQGTKLEIK

SEQ ID NO: 151 - protein, 107 aa, synthetic construct
  REGION 1..107: note = VL domain of Datopotamab IgG histidine scanning variant #23
DIQMTQSPSSLSASVGDRVTITCKASQDVSTAVAWYQQKPGKAPKLLIYSASYRYTGVPSRFSGSGSGTDFTLTISSLQPEDFAVYYCQQHYHTPLTFGQGTKLEIK

SEQ ID NO: 152 - protein, 107 aa, synthetic construct
  REGION 1..107: note = VL domain of Datopotamab IgG histidine scanning variant #24
DIQMTQSPSSLSASVGDRVTITCKASQDVSTAVAWYQQKPGKAPKLLIYSASYRYTGVPSRFSGSGSGTDFTLTISSLQPEDFAVYYCQQHYIHPLTFGQGTKLEIK

SEQ ID NO: 153 - protein, 107 aa, synthetic construct
  REGION 1..107: note = VL domain of Datopotamab IgG histidine scanning variant #25
DIQMTQSPSSLSASVGDRVTITCKASQDVSTAVAWYQQKPGKAPKLLIYSASYRYTGVPSRFSGSGSGTDFTLTISSLQPEDFAVYYCQQHYITHLTFGQGTKLEIK

SEQ ID NO: 154 - protein, 107 aa, synthetic construct
  REGION 1..107: note = VL domain of Datopotamab IgG histidine scanning variant #26
DIQMTQSPSSLSASVGDRVTITCKASQDVSTAVAWYQQKPGKAPKLLIYSASYRYTGVPSRFSGSGSGTDFTLTISSLQPEDFAVYYCQQHYITPHTFGQGTKLEIK

SEQ ID NO: 155 - protein, 107 aa, synthetic construct
  REGION 1..107: note = VL domain of Datopotamab IgG histidine scanning variant #27
DIQMTQSPSSLSASVGDRVTITCKASQDVSTAVAWYQQKPGKAPKLLIYSASYRYTGVPSRFSGSGSGTDFTLTISSLQPEDFAVYYCQQHYITPLHFGQGTKLEIK

SEQ ID NO: 156 - protein, 119 aa, synthetic construct
  REGION 1..119: note = VH domain of h7E6SVG IgG
QVQLQESGPGLVKPSETLSLTCTVSGGSISSYGVHWIRQPPGKGLEWIGVIWTSGVTDYNSALMGRVTISVDTSKNQFSLKLSSVTAADTAVYYCARDGDYDRYTMDYWGQGTLVTVSS

SEQ ID NO: 157 - protein, 111 aa, synthetic construct
  REGION 1..111: note = VL domain of h7E6SVG IgG
DIVMTQSPDSLAVSLGERATINCRASKSVSTSGYSYMHWYQQKPGQPPKLLIYLASNLESGVPDRFSGSGSGTDFTLTISSLQAEDVAVYYCQHSRELPYTFGQGTKLEIK

SEQ ID NO: 158 - protein, 10 aa, synthetic construct
  REGION 1..10: note = Heavy Chain CDR1 of h7E6SVG IgG
GGSISSYGVH

SEQ ID NO: 159 - protein, 16 aa, synthetic construct
  REGION 1..16: note = Heavy Chain CDR2 of h7E6SVG IgG
VIWTSGVTDYNSALMG

SEQ ID NO: 160 - protein, 13 aa, synthetic construct
  REGION 1..13: note = Heavy Chain CDR3 of h7E6SVG IgG
ARDGDYDRYTMDY

SEQ ID NO: 161 - protein, 15 aa, synthetic construct
  REGION 1..15: note = Light Chain CDR1 of h7E6SVG IgG
RASKSVSTSGYSYMH

SEQ ID NO: 162 - protein, 7 aa, synthetic construct
  REGION 1..7: note = Light Chain CDR2 of h7E6SVG IgG
LASNLES

SEQ ID NO: 163 - protein, 9 aa, synthetic construct
  REGION 1..9: note = Light Chain CDR3 of h7E6SVG IgG
QHSRELPYT

SEQ ID NO: 164 - protein, 119 aa, synthetic construct
  REGION 1..119: note = VH domain of h7E6SVG IgG histidine scanning variant #1
QVQLQESGPGLVKPSETLSLTCTVSHGSISSYGVHWIRQPPGKGLEWIGVIWTSGVTDYNSALMGRVTISVDTSKNQFSLKLSSVTAADTAVYYCARDGDYDRYTMDYWGQGTLVTVSS

SEQ ID NO: 165 - protein, 119 aa, synthetic construct
  REGION 1..119: note = VH domain of h7E6SVG IgG histidine scanning variant #2
QVQLQESGPGLVKPSETLSLTCTVSGHSISSYGVHWIRQPPGKGLEWIGVIWTSGVTDYNSALMGRVTISVDTSKNQFSLKLSSVTAADTAVYYCARDGDYDRYTMDYWGQGTLVTVSS

SEQ ID NO: 166 - protein, 119 aa, synthetic construct
  REGION 1..119: note = VH domain of h7E6SVG IgG histidine scanning variant #3
QVQLQESGPGLVKPSETLSLTCTVSGGHISSYGVHWIRQPPGKGLEWIGVIWTSGVTDYNSALMGRVTISVDTSKNQFSLKLSSVTAADTAVYYCARDGDYDRYTMDYWGQGTLVTVSS

SEQ ID NO: 167 - protein, 119 aa, synthetic construct
  REGION 1..119: note = VH domain of h7E6SVG IgG histidine scanning variant #4
QVQLQESGPGLVKPSETLSLTCTVSGGSHSSYGVHWIRQPPGKGLEWIGVIWTSGVTDYNSALMGRVTISVDTSKNQFSLKLSSVTAADTAVYYCARDGDYDRYTMDYWGQGTLVTVSS

SEQ ID NO: 168 - protein, 119 aa, synthetic construct
  REGION 1..119: note = VH domain of h7E6SVG IgG histidine scanning variant #5
QVQLQESGPGLVKPSETLSLTCTVSGGSIHSYGVHWIRQPPGKGLEWIGVIWTSGVTDYNSALMGRVTISVDTSKNQFSLKLSSVTAADTAVYYCARDGDYDRYTMDYWGQGTLVTVSS

SEQ ID NO: 169 - protein, 119 aa, synthetic construct
  REGION 1..119: note = VH domain of h7E6SVG IgG histidine scanning variant #6
QVQLQESGPGLVKPSETLSLTCTVSGGSISHYGVHWIRQPPGKGLEWIGVIWTSGVTDYNSALMGRVTISVDTSKNQFSLKLSSVTAADTAVYYCARDGDYDRYTMDYWGQGTLVTVSS

SEQ ID NO: 170 - protein, 119 aa, synthetic construct
  REGION 1..119: note = VH domain of h7E6SVG IgG histidine scanning variant #7
QVQLQESGPGLVKPSETLSLTCTVSGGSISSHGVHWIRQPPGKGLEWIGVIWTSGVTDYNSALMGRVTISVDTSKNQFSLKLSSVTAADTAVYYCARDGDYDRYTMDYWGQGTLVTVSS

SEQ ID NO: 171 - protein, 119 aa, synthetic construct
  REGION 1..119: note = VH domain of h7E6SVG IgG histidine scanning variant #8
QVQLQESGPGLVKPSETLSLTCTVSGGSISSYHVHWIRQPPGKGLEWIGVIWTSGVTDYNSALMGRVTISVDTSKNQFSLKLSSVTAADTAVYYCARDGDYDRYTMDYWGQGTLVTVSS

SEQ ID NO: 172 - protein, 119 aa, synthetic construct
  REGION 1..119: note = VH domain of h7E6SVG IgG histidine scanning variant #9
QVQLQESGPGLVKPSETLSLTCTVSGGSISSYGHHWIRQPPGKGLEWIGVIWTSGVTDYNSALMGRVTISVDTSKNQFSLKLSSVTAADTAVYYCARDGDYDRYTMDYWGQGTLVTVSS

SEQ ID NO: 173 - protein, 119 aa, synthetic construct
  REGION 1..119: note = VH domain of h7E6SVG IgG histidine scanning variant #10
QVQLQESGPGLVKPSETLSLTCTVSGGSISSYGVAWIRQPPGKGLEWIGVIWTSGVTDYNSALMGRVTISVDTSKNQFSLKLSSVTAADTAVYYCARDGDYDRYTMDYWGQGTLVTVSS

SEQ ID NO: 174 - protein, 119 aa, synthetic construct
  REGION 1..119: note = VH domain of h7E6SVG IgG histidine scanning variant #11
QVQLQESGPGLVKPSETLSLTCTVSGGSISSYGVHWIRQPPGKGLEWIGHIWTSGVTDYNSALMGRVTISVDTSKNQFSLKLSSVTAADTAVYYCARDGDYDRYTMDYWGQGTLVTVSS

SEQ ID NO: 175 - protein, 119 aa, synthetic construct
  REGION 1..119: note = VH domain of h7E6SVG IgG histidine scanning variant #12
QVQLQESGPGLVKPSETLSLTCTVSGGSISSYGVHWIRQPPGKGLEWIGVHWTSGVTDYNSALMGRVTISVDTSKNQFSLKLSSVTAADTAVYYCARDGDYDRYTMDYWGQGTLVTVSS

SEQ ID NO: 176 - protein, 119 aa, synthetic construct
  REGION 1..119: note = VH domain of h7E6SVG IgG histidine scanning variant #13
QVQLQESGPGLVKPSETLSLTCTVSGGSISSYGVHWIRQPPGKGLEWIGVIHTSGVTDYNSALMGRVTISVDTSKNQFSLKLSSVTAADTAVYYCARDGDYDRYTMDYWGQGTLVTVSS

SEQ ID NO: 177 - protein, 119 aa, synthetic construct
  REGION 1..119: note = VH domain of h7E6SVG IgG histidine scanning variant #14
QVQLQESGPGLVKPSETLSLTCTVSGGSISSYGVHWIRQPPGKGLEWIGVIWHSGVTDYNSALMGRVTISVDTSKNQFSLKLSSVTAADTAVYYCARDGDYDRYTMDYWGQGTLVTVSS

SEQ ID NO: 178 - protein, 119 aa, synthetic construct
  REGION 1..119: note = VH domain of h7E6SVG IgG histidine scanning variant #15
QVQLQESGPGLVKPSETLSLTCTVSGGSISSYGVHWIRQPPGKGLEWIGVIWTHGVTDYNSALMGRVTISVDTSKNQFSLKLSSVTAADTAVYYCARDGDYDRYTMDYWGQGTLVTVSS

SEQ ID NO: 179 - protein, 119 aa, synthetic construct
  REGION 1..119: note = VH domain of h7E6SVG IgG histidine scanning variant #16
QVQLQESGPGLVKPSETLSLTCTVSGGSISSYGVHWIRQPPGKGLEWIGVIWTSHVTDYNSALMGRVTISVDTSKNQFSLKLSSVTAADTAVYYCARDGDYDRYTMDYWGQGTLVTVSS

SEQ ID NO: 180 - protein, 119 aa, synthetic construct
  REGION 1..119: note = VH domain of h7E6SVG IgG histidine scanning variant #17
QVQLQESGPGLVKPSETLSLTCTVSGGSISSYGVHWIRQPPGKGLEWIGVIWTSGHTDYNSALMGRVTISVDTSKNQFSLKLSSVTAADTAVYYCARDGDYDRYTMDYWGQGTLVTVSS

SEQ ID NO: 181 - protein, 119 aa, synthetic construct
  REGION 1..119: note = VH domain of h7E6SVG IgG histidine scanning variant #18
QVQLQESGPGLVKPSETLSLTCTVSGGSISSYGVHWIRQPPGKGLEWIGVIWTSGVHDYNSALMGRVTISVDTSKNQFSLKLSSVTAADTAVYYCARDGDYDRYTMDYWGQGTLVTVSS

SEQ ID NO: 182 - protein, 119 aa, synthetic construct
  REGION 1..119: note = VH domain of h7E6SVG IgG histidine scanning variant #19
QVQLQESGPGLVKPSETLSLTCTVSGGSISSYGVHWIRQPPGKGLEWIGVIWTSGVTHYNSALMGRVTISVDTSKNQFSLKLSSVTAADTAVYYCARDGDYDRYTMDYWGQGTLVTVSS

SEQ ID NO: 183 - protein, 119 aa, synthetic construct
  REGION 1..119: note = VH domain of h7E6SVG IgG histidine scanning variant #20
QVQLQESGPGLVKPSETLSLTCTVSGGSISSYGVHWIRQPPGKGLEWIGVIWTSGVTDHNSALMGRVTISVDTSKNQFSLKLSSVTAADTAVYYCARDGDYDRYTMDYWGQGTLVTVSS

SEQ ID NO: 184 - protein, 119 aa, synthetic construct
  REGION 1..119: note = VH domain of h7E6SVG IgG histidine scanning variant #21
QVQLQESGPGLVKPSETLSLTCTVSGGSISSYGVHWIRQPPGKGLEWIGVIWTSGVTDYHSALMGRVTISVDTSKNQFSLKLSSVTAADTAVYYCARDGDYDRYTMDYWGQGTLVTVSS

SEQ ID NO: 185 - protein, 119 aa, synthetic construct
  REGION 1..119: note = VH domain of h7E6SVG IgG histidine scanning variant #22
QVQLQESGPGLVKPSETLSLTCTVSGGSISSYGVHWIRQPPGKGLEWIGVIWTSGVTDYNHALMGRVTISVDTSKNQFSLKLSSVTAADTAVYYCARDGDYDRYTMDYWGQGTLVTVSS

SEQ ID NO: 186 - protein, 119 aa, synthetic construct
  REGION 1..119: note = VH domain of h7E6SVG IgG histidine scanning variant #23
QVQLQESGPGLVKPSETLSLTCTVSGGSISSYGVHWIRQPPGKGLEWIGVIWTSGVTDYNSHLMGRVTISVDTSKNQFSLKLSSVTAADTAVYYCARDGDYDRYTMDYWGQGTLVTVSS

SEQ ID NO: 187 - protein, 119 aa, synthetic construct
  REGION 1..119: note = VH domain of h7E6SVG IgG histidine scanning variant #24
QVQLQESGPGLVKPSETLSLTCTVSGGSISSYGVHWIRQPPGKGLEWIGVIWTSGVTDYNSAHMGRVTISVDTSKNQFSLKLSSVTAADTAVYYCARDGDYDRYTMDYWGQGTLVTVSS

SEQ ID NO: 188 - protein, 119 aa, synthetic construct
  REGION 1..119: note = VH domain of h7E6SVG IgG histidine scanning variant #25
QVQLQESGPGLVKPSETLSLTCTVSGGSISSYGVHWIRQPPGKGLEWIGVIWTSGVTDYNSALHGRVTISVDTSKNQFSLKLSSVTAADTAVYYCARDGDYDRYTMDYWGQGTLVTVSS

SEQ ID NO: 189 - protein, 119 aa, synthetic construct
  REGION 1..119: note = VH domain of h7E6SVG IgG histidine scanning variant #26
QVQLQESGPGLVKPSETLSLTCTVSGGSISSYGVHWIRQPPGKGLEWIGVIWTSGVTDYNSALMHRVTISVDTSKNQFSLKLSSVTAADTAVYYCARDGDYDRYTMDYWGQGTLVTVSS

SEQ ID NO: 190 - protein, 119 aa, synthetic construct
  REGION 1..119: note = VH domain of h7E6SVG IgG histidine scanning variant #27
QVQLQESGPGLVKPSETLSLTCTVSGGSISSYGVHWIRQPPGKGLEWIGVIWTSGVTDYNSALMGRVTISVDTSKNQFSLKLSSVTAADTAVYYCHRDGDYDRYTMDYWGQGTLVTVSS

SEQ ID NO: 191 - protein, 119 aa, synthetic construct
  REGION 1..119: note = VH domain of h7E6SVG IgG histidine scanning variant #28
QVQLQESGPGLVKPSETLSLTCTVSGGSISSYGVHWIRQPPGKGLEWIGVIWTSGVTDYNSALMGRVTISVDTSKNQFSLKLSSVTAADTAVYYCAHDGDYDRYTMDYWGQGTLVTVSS

SEQ ID NO: 192 - protein, 119 aa, synthetic construct
  REGION 1..119: note = VH domain of h7E6SVG IgG histidine scanning variant #29
QVQLQESGPGLVKPSETLSLTCTVSGGSISSYGVHWIRQPPGKGLEWIGVIWTSGVTDYNSALMGRVTISVDTSKNQFSLKLSSVTAADTAVYYCARHGDYDRYTMDYWGQGTLVTVSS

SEQ ID NO: 193 - protein, 119 aa, synthetic construct
  REGION 1..119: note = VH domain of h7E6SVG IgG histidine scanning variant #30
QVQLQESGPGLVKPSETLSLTCTVSGGSISSYGVHWIRQPPGKGLEWIGVIWTSGVTDYNSALMGRVTISVDTSKNQFSLKLSSVTAADTAVYYCARDHDYDRYTMDYWGQGTLVTVSS

SEQ ID NO: 194 - protein, 119 aa, synthetic construct
  REGION 1..119: note = VH domain of h7E6SVG IgG histidine scanning variant #31
QVQLQESGPGLVKPSETLSLTCTVSGGSISSYGVHWIRQPPGKGLEWIGVIWTSGVTDYNSALMGRVTISVDTSKNQFSLKLSSVTAADTAVYYCARDGHYDRYTMDYWGQGTLVTVSS

SEQ ID NO: 195 - protein, 119 aa, synthetic construct
  REGION 1..119: note = VH domain of h7E6SVG IgG histidine scanning variant #32
QVQLQESGPGLVKPSETLSLTCTVSGGSISSYGVHWIRQPPGKGLEWIGVIWTSGVTDYNSALMGRVTISVDTSKNQFSLKLSSVTAADTAVYYCARDGDHDRYTMDYWGQGTLVTVSS

SEQ ID NO: 196 - protein, 119 aa, synthetic construct
  REGION 1..119: note = VH domain of h7E6SVG IgG histidine scanning variant #33
QVQLQESGPGLVKPSETLSLTCTVSGGSISSYGVHWIRQPPGKGLEWIGVIWTSGVTDYNSALMGRVTISVDTSKNQFSLKLSSVTAADTAVYYCARDGDYHRYTMDYWGQGTLVTVSS

SEQ ID NO: 197 - protein, 119 aa, synthetic construct
  REGION 1..119: note = VH domain of h7E6SVG IgG histidine scanning variant #34
QVQLQESGPGLVKPSETLSLTCTVSGGSISSYGVHWIRQPPGKGLEWIGVIWTSGVTDYNSALMGRVTISVDTSKNQFSLKLSSVTAADTAVYYCARDGDYDHYTMDYWGQGTLVTVSS

SEQ ID NO: 198 - protein, 119 aa, synthetic construct
  REGION 1..119: note = VH domain of h7E6SVG IgG histidine scanning variant #35
QVQLQESGPGLVKPSETLSLTCTVSGGSISSYGVHWIRQPPGKGLEWIGVIWTSGVTDYNSALMGRVTISVDTSKNQFSLKLSSVTAADTAVYYCARDGDYDRHTMDYWGQGTLVTVSS

SEQ ID NO: 199 - protein, 119 aa, synthetic construct
  REGION 1..119: note = VH domain of h7E6SVG IgG histidine scanning variant #36
QVQLQESGPGLVKPSETLSLTCTVSGGSISSYGVHWIRQPPGKGLEWIGVIWTSGVTDYNSALMGRVTISVDTSKNQFSLKLSSVTAADTAVYYCARDGDYDRYHMDYWGQGTLVTVSS

SEQ ID NO: 200 - protein, 119 aa, synthetic construct
  REGION 1..119: note = VH domain of h7E6SVG IgG histidine scanning variant #37
QVQLQESGPGLVKPSETLSLTCTVSGGSISSYGVHWIRQPPGKGLEWIGVIWTSGVTDYNSALMGRVTISVDTSKNQFSLKLSSVTAADTAVYYCARDGDYDRYTHDYWGQGTLVTVSS

SEQ ID NO: 201 - protein, 119 aa, synthetic construct
  REGION 1..119: note = VH domain of h7E6SVG IgG histidine scanning variant #38
QVQLQESGPGLVKPSETLSLTCTVSGGSISSYGVHWIRQPPGKGLEWIGVIWTSGVTDYNSALMGRVTISVDTSKNQFSLKLSSVTAADTAVYYCARDGDYDRYTMHYWGQGTLVTVSS

SEQ ID NO: 202 - protein, 119 aa, synthetic construct
  REGION 1..119: note = VH domain of h7E6SVG IgG histidine scanning variant #39
QVQLQESGPGLVKPSETLSLTCTVSGGSISSYGVHWIRQPPGKGLEWIGVIWTSGVTDYNSALMGRVTISVDTSKNQFSLKLSSVTAADTAVYYCARDGDYDRYTMDHWGQGTLVTVSS

SEQ ID NO: 203 - protein, 111 aa, synthetic construct
  REGION 1..111: note = VL domain of h7E6SVG IgG histidine scanning variant #1
DIVMTQSPDSLAVSLGERATINCHASKSVSTSGYSYMHWYQQKPGQPPKLLIYLASNLESGVPDRFSGSGSGTDFTLTISSLQAEDVAVYYCQHSRELPYTFGQGTKLEIK

SEQ ID NO: 204 - protein, 111 aa, synthetic construct
  REGION 1..111: note = VL domain of h7E6SVG IgG histidine scanning variant #2
DIVMTQSPDSLAVSLGERATINCRHSKSVSTSGYSYMHWYQQKPGQPPKLLIYLASNLESGVPDRFSGSGSGTDFTLTISSLQAEDVAVYYCQHSRELPYTFGQGTKLEIK

SEQ ID NO: 205 - protein, 111 aa, synthetic construct
  REGION 1..111: note = VL domain of h7E6SVG IgG histidine scanning variant #3
DIVMTQSPDSLAVSLGERATINCRAHKSVSTSGYSYMHWYQQKPGQPPKLLIYLASNLESGVPDRFSGSGSGTDFTLTISSLQAEDVAVYYCQHSRELPYTFGQGTKLEIK

SEQ ID NO: 206 - protein, 111 aa, synthetic construct
  REGION 1..111: note = VL domain of h7E6SVG IgG histidine scanning variant #4
DIVMTQSPDSLAVSLGERATINCRASHSVSTSGYSYMHWYQQKPGQPPKLLIYLASNLESGVPDRFSGSGSGTDFTLTISSLQAEDVAVYYCQHSRELPYTFGQGTKLEIK

SEQ ID NO: 207 - protein, 111 aa, synthetic construct
  REGION 1..111: note = VL domain of h7E6SVG IgG histidine scanning variant #5
DIVMTQSPDSLAVSLGERATINCRASKHVSTSGYSYMHWYQQKPGQPPKLLIYLASNLESGVPDRFSGSGSGTDFTLTISSLQAEDVAVYYCQHSRELPYTFGQGTKLEIK

SEQ ID NO: 208 - protein, 111 aa, synthetic construct
  REGION 1..111: note = VL domain of h7E6SVG IgG histidine scanning variant #6
DIVMTQSPDSLAVSLGERATINCRASKSHSTSGYSYMHWYQQKPGQPPKLLIYLASNLESGVPDRFSGSGSGTDFTLTISSLQAEDVAVYYCQHSRELPYTFGQGTKLEIK

SEQ ID NO: 209 - protein, 111 aa, synthetic construct
  REGION 1..111: note = VL domain of h7E6SVG IgG histidine scanning variant #7
DIVMTQSPDSLAVSLGERATINCRASKSVHTSGYSYMHWYQQKPGQPPKLLIYLASNLESGVPDRFSGSGSGTDFTLTISSLQAEDVAVYYCQHSRELPYTFGQGTKLEIK

SEQ ID NO: 210 - protein, 111 aa, synthetic construct
  REGION 1..111: note = VL domain of h7E6SVG IgG histidine scanning variant #8
DIVMTQSPDSLAVSLGERATINCRASKSVSHSGYSYMHWYQQKPGQPPKLLIYLASNLESGVPDRFSGSGSGTDFTLTISSLQAEDVAVYYCQHSRELPYTFGQGTKLEIK

SEQ ID NO: 211 - protein, 111 aa, synthetic construct
  REGION 1..111: note = VL domain of h7E6SVG IgG histidine scanning variant #9
DIVMTQSPDSLAVSLGERATINCRASKSVSTHGYSYMHWYQQKPGQPPKLLIYLASNLESGVPDRFSGSGSGTDFTLTISSLQAEDVAVYYCQHSRELPYTFGQGTKLEIK

SEQ ID NO: 212 - protein, 111 aa, synthetic construct
  REGION 1..111: note = VL domain of h7E6SVG IgG histidine scanning variant #10
DIVMTQSPDSLAVSLGERATINCRASKSVSTSHYSYMHWYQQKPGQPPKLLIYLASNLESGVPDRFSGSGSGTDFTLTISSLQAEDVAVYYCQHSRELPYTFGQGTKLEIK

SEQ ID NO: 213 - protein, 111 aa, synthetic construct
  REGION 1..111: note = VL domain of h7E6SVG IgG histidine scanning variant #11
DIVMTQSPDSLAVSLGERATINCRASKSVSTSGHSYMHWYQQKPGQPPKLLIYLASNLESGVPDRFSGSGSGTDFTLTISSLQAEDVAVYYCQHSRELPYTFGQGTKLEIK

SEQ ID NO: 214 - protein, 111 aa, synthetic construct
  REGION 1..111: note = VL domain of h7E6SVG IgG histidine scanning variant #12
DIVMTQSPDSLAVSLGERATINCRASKSVSTSGYHYMHWYQQKPGQPPKLLIYLASNLESGVPDRFSGSGSGTDFTLTISSLQAEDVAVYYCQHSRELPYTFGQGTKLEIK

SEQ ID NO: 215 - protein, 111 aa, synthetic construct
  REGION 1..111: note = VL domain of h7E6SVG IgG histidine scanning variant #13
DIVMTQSPDSLAVSLGERATINCRASKSVSTSGYSHMHWYQQKPGQPPKLLIYLASNLESGVPDRFSGSGSGTDFTLTISSLQAEDVAVYYCQHSRELPYTFGQGTKLEIK

SEQ ID NO: 216 - protein, 111 aa, synthetic construct
  REGION 1..111: note = VL domain of h7E6SVG IgG histidine scanning variant #14
DIVMTQSPDSLAVSLGERATINCRASKSVSTSGYSYHHWYQQKPGQPPKLLIYLASNLESGVPDRFSGSGSGTDFTLTISSLQAEDVAVYYCQHSRELPYTFGQGTKLEIK

SEQ ID NO: 217 - protein, 111 aa, synthetic construct
  REGION 1..111: note = VL domain of h7E6SVG IgG histidine scanning variant #15
DIVMTQSPDSLAVSLGERATINCRASKSVSTSGYSYMAWYQQKPGQPPKLLIYLASNLESGVPDRFSGSGSGTDFTLTISSLQAEDVAVYYCQHSRELPYTFGQGTKLEIK

SEQ ID NO: 218 - protein, 111 aa, synthetic construct
  REGION 1..111: note = VL domain of h7E6SVG IgG histidine scanning variant #16
DIVMTQSPDSLAVSLGERATINCRASKSVSTSGYSYMHWYQQKPGQPPKLLIYHASNLESGVPDRFSGSGSGTDFTLTISSLQAEDVAVYYCQHSRELPYTFGQGTKLEIK

SEQ ID NO: 219 - protein, 111 aa, synthetic construct
  REGION 1..111: note = VL domain of h7E6SVG IgG histidine scanning variant #17
DIVMTQSPDSLAVSLGERATINCRASKSVSTSGYSYMHWYQQKPGQPPKLLIYLHSNLESGVPDRFSGSGSGTDFTLTISSLQAEDVAVYYCQHSRELPYTFGQGTKLEIK

SEQ ID NO: 220 - protein, 111 aa, synthetic construct
  REGION 1..111: note = VL domain of h7E6SVG IgG histidine scanning variant #18
DIVMTQSPDSLAVSLGERATINCRASKSVSTSGYSYMHWYQQKPGQPPKLLIYLAHNLESGVPDRFSGSGSGTDFTLTISSLQAEDVAVYYCQHSRELPYTFGQGTKLEIK

SEQ ID NO: 221 - protein, 111 aa, synthetic construct
  REGION 1..111: note = VL domain of h7E6SVG IgG histidine scanning variant #19
DIVMTQSPDSLAVSLGERATINCRASKSVSTSGYSYMHWYQQKPGQPPKLLIYLASHLESGVPDRFSGSGSGTDFTLTISSLQAEDVAVYYCQHSRELPYTFGQGTKLEIK

SEQ ID NO: 222 - protein, 111 aa, synthetic construct
  REGION 1..111: note = VL domain of h7E6SVG IgG histidine scanning variant #20
DIVMTQSPDSLAVSLGERATINCRASKSVSTSGYSYMHWYQQKPGQPPKLLIYLASNHESGVPDRFSGSGSGTDFTLTISSLQAEDVAVYYCQHSRELPYTFGQGTKLEIK

SEQ ID NO: 223 - protein, 111 aa, synthetic construct
  REGION 1..111: note = VL domain of h7E6SVG IgG histidine scanning variant #21
DIVMTQSPDSLAVSLGERATINCRASKSVSTSGYSYMHWYQQKPGQPPKLLIYLASNLHSGVPDRFSGSGSGTDFTLTISSLQAEDVAVYYCQHSRELPYTFGQGTKLEIK

SEQ ID NO: 224 - protein, 111 aa, synthetic construct
  REGION 1..111: note = VL domain of h7E6SVG IgG histidine scanning variant #22
DIVMTQSPDSLAVSLGERATINCRASKSVSTSGYSYMHWYQQKPGQPPKLLIYLASNLEHGVPDRFSGSGSGTDFTLTISSLQAEDVAVYYCQHSRELPYTFGQGTKLEIK

SEQ ID NO: 225 - protein, 111 aa, synthetic construct
  REGION 1..111: note = VL domain of h7E6SVG IgG histidine scanning variant #23
DIVMTQSPDSLAVSLGERATINCRASKSVSTSGYSYMHWYQQKPGQPPKLLIYLASNLESGVPDRFSGSGSGTDFTLTISSLQAEDVAVYYCHHSRELPYTFGQGTKLEIK

SEQ ID NO: 226 - protein, 111 aa, synthetic construct
  REGION 1..111: note = VL domain of h7E6SVG IgG histidine scanning variant #24
DIVMTQSPDSLAVSLGERATINCRASKSVSTSGYSYMHWYQQKPGQPPKLLIYLASNLESGVPDRFSGSGSGTDFTLTISSLQAEDVAVYYCQASRELPYTFGQGTKLEIK

SEQ ID NO: 227 - protein, 111 aa, synthetic construct
  REGION 1..111: note = VL domain of h7E6SVG IgG histidine scanning variant #25
DIVMTQSPDSLAVSLGERATINCRASKSVSTSGYSYMHWYQQKPGQPPKLLIYLASNLESGVPDRFSGSGSGTDFTLTISSLQAEDVAVYYCQHHRELPYTFGQGTKLEIK

SEQ ID NO: 228 - protein, 111 aa, synthetic construct
  REGION 1..111: note = VL domain of h7E6SVG IgG histidine scanning variant #26
DIVMTQSPDSLAVSLGERATINCRASKSVSTSGYSYMHWYQQKPGQPPKLLIYLASNLESGVPDRFSGSGSGTDFTLTISSLQAEDVAVYYCQHSHELPYTFGQGTKLEIK

SEQ ID NO: 229 - protein, 111 aa, synthetic construct
  REGION 1..111: note = VL domain of h7E6SVG IgG histidine scanning variant #27
DIVMTQSPDSLAVSLGERATINCRASKSVSTSGYSYMHWYQQKPGQPPKLLIYLASNLESGVPDRFSGSGSGTDFTLTISSLQAEDVAVYYCQHSRHLPYTFGQGTKLEIK

SEQ ID NO: 230 - protein, 111 aa, synthetic construct
  REGION 1..111: note = VL domain of h7E6SVG IgG histidine scanning variant #28
DIVMTQSPDSLAVSLGERATINCRASKSVSTSGYSYMHWYQQKPGQPPKLLIYLASNLESGVPDRFSGSGSGTDFTLTISSLQAEDVAVYYCQHSREHPYTFGQGTKLEIK

SEQ ID NO: 231 - protein, 111 aa, synthetic construct
  REGION 1..111: note = VL domain of h7E6SVG IgG histidine scanning variant #29
DIVMTQSPDSLAVSLGERATINCRASKSVSTSGYSYMHWYQQKPGQPPKLLIYLASNLESGVPDRFSGSGSGTDFTLTISSLQAEDVAVYYCQHSRELHYTFGQGTKLEIK

SEQ ID NO: 232 - protein, 111 aa, synthetic construct
  REGION 1..111: note = VL domain of h7E6SVG IgG histidine scanning variant #30
DIVMTQSPDSLAVSLGERATINCRASKSVSTSGYSYMHWYQQKPGQPPKLLIYLASNLESGVPDRFSGSGSGTDFTLTISSLQAEDVAVYYCQHSRELPHTFGQGTKLEIK

SEQ ID NO: 233 - protein, 111 aa, synthetic construct
  REGION 1..111: note = VL domain of h7E6SVG IgG histidine scanning variant #31
DIVMTQSPDSLAVSLGERATINCRASKSVSTSGYSYMHWYQQKPGQPPKLLIYLASNLESGVPDRFSGSGSGTDFTLTISSLQAEDVAVYYCQHSRELPYHFGQGTKLEIK

SEQ ID NO: 234 - protein, 119 aa, synthetic construct
  REGION 1..119: note = VH domain of A1,2X4 IgG
QVQLQESGPGLVKPSETLSLTCTVSGFSLTSYGVNWIRQPPGKGLEWIGVMWAGGSTNYNSALMSRLTISKDTSKNQFSLKLSSVTAADTAVYYCARDENWDGAWFAYWGQGTLVTVSS

SEQ ID NO: 235 - protein, 112 aa, synthetic construct
  REGION 1..112: note = VL domain of A1,2X4 IgG
DIVMTQSPDSLAVSLGERATINCKSSQSLLNSGTRKNYLAWYQQKPGQPPKLLISWASSRESGVPDRFSGSGSGTDFTLTISSLQAEDVAVYYCKQSYNLFTFGGGTKVEIK

SEQ ID NO: 236 - protein, 10 aa, synthetic construct
  REGION 1..10: note = Heavy Chain CDR1 of A1,2X4 IgG
GFSLTSYGVN

SEQ ID NO: 237 - protein, 16 aa, synthetic construct
  REGION 1..16: note = Heavy Chain CDR2 of A1,2X4 IgG
VMWAGGSTNYNSALMS

SEQ ID NO: 238 - protein, 13 aa, synthetic construct
  REGION 1..13: note = Heavy Chain CDR3 of A1,2X4 IgG
ARDENWDGAWFAY

SEQ ID NO: 239 - protein, 17 aa, synthetic construct
  REGION 1..17: note = Light Chain CDR1 of A1,2X4 IgG
KSSQSLLNSGTRKNYLA

SEQ ID NO: 240 - protein, 7 aa, synthetic construct
  REGION 1..7: note = Light Chain CDR2 of A1,2X4 IgG
WASSRES

SEQ ID NO: 241 - protein, 8 aa, synthetic construct
  REGION 1..8: note = Light Chain CDR3 of A1,2X4 IgG
KQSYNLFT

SEQ ID NO: 242 - protein, 119 aa, synthetic construct
  REGION 1..119: note = VH domain of A1,2X4 IgG histidine scanning variant #1
QVQLQESGPGLVKPSETLSLTCTVSHFSLTSYGVNWIRQPPGKGLEWIGVMWAGGSTNYNSALMSRLTISKDTSKNQFSLKLSSVTAADTAVYYCARDENWDGAWFAYWGQGTLVTVSS

SEQ ID NO: 243 - protein, 119 aa, synthetic construct
  REGION 1..119: note = VH domain of A1,2X4 IgG histidine scanning variant #2
QVQLQESGPGLVKPSETLSLTCTVSGHSLTSYGVNWIRQPPGKGLEWIGVMWAGGSTNYNSALMSRLTISKDTSKNQFSLKLSSVTAADTAVYYCARDENWDGAWFAYWGQGTLVTVSS

SEQ ID NO: 244 - protein, 119 aa, synthetic construct
  REGION 1..119: note = VH domain of A1,2X4 IgG histidine scanning variant #3
QVQLQESGPGLVKPSETLSLTCTVSGFHLTSYGVNWIRQPPGKGLEWIGVMWAGGSTNYNSALMSRLTISKDTSKNQFSLKLSSVTAADTAVYYCARDENWDGAWFAYWGQGTLVTVSS

SEQ ID NO: 245 - protein, 119 aa, synthetic construct
  REGION 1..119: note = VH domain of A1,2X4 IgG histidine scanning variant #4
QVQLQESGPGLVKPSETLSLTCTVSGFSHTSYGVNWIRQPPGKGLEWIGVMWAGGSTNYNSALMSRLTISKDTSKNQFSLKLSSVTAADTAVYYCARDENWDGAWFAYWGQGTLVTVSS

SEQ ID NO: 246 - protein, 119 aa, synthetic construct
  REGION 1..119: note = VH domain of A1,2X4 IgG histidine scanning variant #5
QVQLQESGPGLVKPSETLSLTCTVSGFSLHSYGVNWIRQPPGKGLEWIGVMWAGGSTNYNSALMSRLTISKDTSKNQFSLKLSSVTAADTAVYYCARDENWDGAWFAYWGQGTLVTVSS

SEQ ID NO: 247 - protein, 119 aa, synthetic construct
  REGION 1..119: note = VH domain of A1,2X4 IgG histidine scanning variant #6
QVQLQESGPGLVKPSETLSLTCTVSGFSLTHYGVNWIRQPPGKGLEWIGVMWAGGSTNYNSALMSRLTISKDTSKNQFSLKLSSVTAADTAVYYCARDENWDGAWFAYWGQGTLVTVSS

SEQ ID NO: 248 - protein, 119 aa, synthetic construct
  REGION 1..119: note = VH domain of A1,2X4 IgG histidine scanning variant #7
QVQLQESGPGLVKPSETLSLTCTVSGFSLTSHGVNWIRQPPGKGLEWIGVMWAGGSTNYNSALMSRLTISKDTSKNQFSLKLSSVTAADTAVYYCARDENWDGAWFAYWGQGTLVTVSS

SEQ ID NO: 249 - protein, 119 aa, synthetic construct
  REGION 1..119: note = VH domain of A1,2X4 IgG histidine scanning variant #8
QVQLQESGPGLVKPSETLSLTCTVSGFSLTSYHVNWIRQPPGKGLEWIGVMWAGGSTNYNSALMSRLTISKDTSKNQFSLKLSSVTAADTAVYYCARDENWDGAWFAYWGQGTLVTVSS

SEQ ID NO: 250 - protein, 119 aa, synthetic construct
  REGION 1..119: note = VH domain of A1,2X4 IgG histidine scanning variant #9
QVQLQESGPGLVKPSETLSLTCTVSGFSLTSYGHNWIRQPPGKGLEWIGVMWAGGSTNYNSALMSRLTISKDTSKNQFSLKLSSVTAADTAVYYCARDENWDGAWFAYWGQGTLVTVSS

SEQ ID NO: 251 - protein, 119 aa, synthetic construct
  REGION 1..119: note = VH domain of A1,2X4 IgG histidine scanning variant #10
QVQLQESGPGLVKPSETLSLTCTVSGFSLTSYGVHWIRQPPGKGLEWIGVMWAGGSTNYNSALMSRLTISKDTSKNQFSLKLSSVTAADTAVYYCARDENWDGAWFAYWGQGTLVTVSS

SEQ ID NO: 252 - protein, 119 aa, synthetic construct
  REGION 1..119: note = VH domain of A1,2X4 IgG histidine scanning variant #11
QVQLQESGPGLVKPSETLSLTCTVSGFSLTSYGVNWIRQPPGKGLEWIGHMWAGGSTNYNSALMSRLTISKDTSKNQFSLKLSSVTAADTAVYYCARDENWDGAWFAYWGQGTLVTVSS

SEQ ID NO: 253 - protein, 119 aa, synthetic construct
  REGION 1..119: note = VH domain of A1,2X4 IgG histidine scanning variant #12
QVQLQESGPGLVKPSETLSLTCTVSGFSLTSYGVNWIRQPPGKGLEWIGVHWAGGSTNYNSALMSRLTISKDTSKNQFSLKLSSVTAADTAVYYCARDENWDGAWFAYWGQGTLVTVSS

SEQ ID NO: 254 - protein, 119 aa, synthetic construct
  REGION 1..119: note = VH domain of A1,2X4 IgG histidine scanning variant #13
QVQLQESGPGLVKPSETLSLTCTVSGFSLTSYGVNWIRQPPGKGLEWIGVMHAGGSTNYNSALMSRLTISKDTSKNQFSLKLSSVTAADTAVYYCARDENWDGAWFAYWGQGTLVTVSS

SEQ ID NO: 255 - protein, 119 aa, synthetic construct
  REGION 1..119: note = VH domain of A1,2X4 IgG histidine scanning variant #14
QVQLQESGPGLVKPSETLSLTCTVSGFSLTSYGVNWIRQPPGKGLEWIGVMWHGGSTNYNSALMSRLTISKDTSKNQFSLKLSSVTAADTAVYYCARDENWDGAWFAYWGQGTLVTVSS

SEQ ID NO: 256 - protein, 119 aa, synthetic construct
  REGION 1..119: note = VH domain of A1,2X4 IgG histidine scanning variant #15
QVQLQESGPGLVKPSETLSLTCTVSGFSLTSYGVNWIRQPPGKGLEWIGVMWAHGSTNYNSALMSRLTISKDTSKNQFSLKLSSVTAADTAVYYCARDENWDGAWFAYWGQGTLVTVSS

SEQ ID NO: 257 - protein, 119 aa, synthetic construct
  REGION 1..119: note = VH domain of A1,2X4 IgG histidine scanning variant #16
QVQLQESGPGLVKPSETLSLTCTVSGFSLTSYGVNWIRQPPGKGLEWIGVMWAGHSTNYNSALMSRLTISKDTSKNQFSLKLSSVTAADTAVYYCARDENWDGAWFAYWGQGTLVTVSS

SEQ ID NO: 258 - protein, 119 aa, synthetic construct
  REGION 1..119: note = VH domain of A1,2X4 IgG histidine scanning variant #17
QVQLQESGPGLVKPSETLSLTCTVSGFSLTSYGVNWIRQPPGKGLEWIGVMWAGGHTNYNSALMSRLTISKDTSKNQFSLKLSSVTAADTAVYYCARDENWDGAWFAYWGQGTLVTVSS

SEQ ID NO: 259 - protein, 119 aa, synthetic construct
  REGION 1..119: note = VH domain of A1,2X4 IgG histidine scanning variant #18
QVQLQESGPGLVKPSETLSLTCTVSGFSLTSYGVNWIRQPPGKGLEWIGVMWAGGSHNYNSALMSRLTISKDTSKNQFSLKLSSVTAADTAVYYCARDENWDGAWFAYWGQGTLVTVSS

SEQ ID NO: 260 - protein, 119 aa, synthetic construct
  REGION 1..119: note = VH domain of A1,2X4 IgG histidine scanning variant #19
QVQLQESGPGLVKPSETLSLTCTVSGFSLTSYGVNWIRQPPGKGLEWIGVMWAGGSTHYNSALMSRLTISKDTSKNQFSLKLSSVTAADTAVYYCARDENWDGAWFAYWGQGTLVTVSS

SEQ ID NO: 261 - protein, 119 aa, synthetic construct
  REGION 1..119: note = VH domain of A1,2X4 IgG histidine scanning variant #20
QVQLQESGPGLVKPSETLSLTCTVSGFSLTSYGVNWIRQPPGKGLEWIGVMWAGGSTNHNSALMSRLTISKDTSKNQFSLKLSSVTAADTAVYYCARDENWDGAWFAYWGQGTLVTVSS

SEQ ID NO: 262 - protein, 119 aa, synthetic construct
  REGION 1..119: note = VH domain of A1,2X4 IgG histidine scanning variant #21
QVQLQESGPGLVKPSETLSLTCTVSGFSLTSYGVNWIRQPPGKGLEWIGVMWAGGSTNYHSALMSRLTISKDTSKNQFSLKLSSVTAADTAVYYCARDENWDGAWFAYWGQGTLVTVSS

SEQ ID NO: 263 - protein, 119 aa, synthetic construct
  REGION 1..119: note = VH domain of A1,2X4 IgG histidine scanning variant #22
QVQLQESGPGLVKPSETLSLTCTVSGFSLTSYGVNWIRQPPGKGLEWIGVMWAGGSTNYNHALMSRLTISKDTSKNQFSLKLSSVTAADTAVYYCARDENWDGAWFAYWGQGTLVTVSS

SEQ ID NO: 264 - protein, 119 aa, synthetic construct
  REGION 1..119: note = VH domain of A1,2X4 IgG histidine scanning variant #23
QVQLQESGPGLVKPSETLSLTCTVSGFSLTSYGVNWIRQPPGKGLEWIGVMWAGGSTNYNSHLMSRLTISKDTSKNQFSLKLSSVTAADTAVYYCARDENWDGAWFAYWGQGTLVTVSS

SEQ ID NO: 265 - protein, 119 aa, synthetic construct
  REGION 1..119: note = VH domain of A1,2X4 IgG histidine scanning variant #24
QVQLQESGPGLVKPSETLSLTCTVSGFSLTSYGVNWIRQPPGKGLEWIGVMWAGGSTNYNSAHMSRLTISKDTSKNQFSLKLSSVTAADTAVYYCARDENWDGAWFAYWGQGTLVTVSS

SEQ ID NO: 266 - protein, 119 aa, synthetic construct
  REGION 1..119: note = VH domain of A1,2X4 IgG histidine scanning variant #25
QVQLQESGPGLVKPSETLSLTCTVSGFSLTSYGVNWIRQPPGKGLEWIGVMWAGGSTNYNSALHSRLTISKDTSKNQFSLKLSSVTAADTAVYYCARDENWDGAWFAYWGQGTLVTVSS

SEQ ID NO: 267 - protein, 119 aa, synthetic construct
  REGION 1..119: note = VH domain of A1,2X4 IgG histidine scanning variant #26
QVQLQESGPGLVKPSETLSLTCTVSGFSLTSYGVNWIRQPPGKGLEWIGVMWAGGSTNYNSALMHRLTISKDTSKNQFSLKLSSVTAADTAVYYCARDENWDGAWFAYWGQGTLVTVSS

SEQ ID NO: 268 - protein, 119 aa, synthetic construct
  REGION 1..119: note = VH domain of A1,2X4 IgG histidine scanning variant #27
QVQLQESGPGLVKPSETLSLTCTVSGFSLTSYGVNWIRQPPGKGLEWIGVMWAGGSTNYNSALMSRLTISKDTSKNQFSLKLSSVTAADTAVYYCHRDENWDGAWFAYWGQGTLVTVSS

SEQ ID NO: 269 - protein, 119 aa, synthetic construct
  REGION 1..119: note = VH domain of A1,2X4 IgG histidine scanning variant #28
QVQLQESGPGLVKPSETLSLTCTVSGFSLTSYGVNWIRQPPGKGLEWIGVMWAGGSTNYNSALMSRLTISKDTSKNQFSLKLSSVTAADTAVYYCAHDENWDGAWFAYWGQGTLVTVSS

SEQ ID NO: 270 - protein, 119 aa, synthetic construct
  REGION 1..119: note = VH domain of A1,2X4 IgG histidine scanning variant #29
QVQLQESGPGLVKPSETLSLTCTVSGFSLTSYGVNWIRQPPGKGLEWIGVMWAGGSTNYNSALMSRLTISKDTSKNQFSLKLSSVTAADTAVYYCARHENWDGAWFAYWGQGTLVTVSS

SEQ ID NO: 271 - protein, 119 aa, synthetic construct
  REGION 1..119: note = VH domain of A1,2X4 IgG histidine scanning variant #30
QVQLQESGPGLVKPSETLSLTCTVSGFSLTSYGVNWIRQPPGKGLEWIGVMWAGGSTNYNSALMSRLTISKDTSKNQFSLKLSSVTAADTAVYYCARDHNWDGAWFAYWGQGTLVTVSS

SEQ ID NO: 272 - protein, 119 aa, synthetic construct
  REGION 1..119: note = VH domain of A1,2X4 IgG histidine scanning variant #31
QVQLQESGPGLVKPSETLSLTCTVSGFSLTSYGVNWIRQPPGKGLEWIGVMWAGGSTNYNSALMSRLTISKDTSKNQFSLKLSSVTAADTAVYYCARDEHWDGAWFAYWGQGTLVTVSS

SEQ ID NO: 273 - protein, 119 aa, synthetic construct
  REGION 1..119: note = VH domain of A1,2X4 IgG histidine scanning variant #32
QVQLQESGPGLVKPSETLSLTCTVSGFSLTSYGVNWIRQPPGKGLEWIGVMWAGGSTNYNSALMSRLTISKDTSKNQFSLKLSSVTAADTAVYYCARDENHDGAWFAYWGQGTLVTVSS

SEQ ID NO: 274 - protein, 119 aa, synthetic construct
  REGION 1..119: note = VH domain of A1,2X4 IgG histidine scanning variant #33
QVQLQESGPGLVKPSETLSLTCTVSGFSLTSYGVNWIRQPPGKGLEWIGVMWAGGSTNYNSALMSRLTISKDTSKNQFSLKLSSVTAADTAVYYCARDENWHGAWFAYWGQGTLVTVSS

SEQ ID NO: 275 - protein, 119 aa, synthetic construct
  REGION 1..119: note = VH domain of A1,2X4 IgG histidine scanning variant #34
QVQLQESGPGLVKPSETLSLTCTVSGFSLTSYGVNWIRQPPGKGLEWIGVMWAGGSTNYNSALMSRLTISKDTSKNQFSLKLSSVTAADTAVYYCARDENWDHAWFAYWGQGTLVTVSS

SEQ ID NO: 276 - protein, 119 aa, synthetic construct
  REGION 1..119: note = VH domain of A1,2X4 IgG histidine scanning variant #35
QVQLQESGPGLVKPSETLSLTCTVSGFSLTSYGVNWIRQPPGKGLEWIGVMWAGGSTNYNSALMSRLTISKDTSKNQFSLKLSSVTAADTAVYYCARDENWDGHWFAYWGQGTLVTVSS

SEQ ID NO: 277 - protein, 119 aa, synthetic construct
  REGION 1..119: note = VH domain of A1,2X4 IgG histidine scanning variant #36
QVQLQESGPGLVKPSETLSLTCTVSGFSLTSYGVNWIRQPPGKGLEWIGVMWAGGSTNYNSALMSRLTISKDTSKNQFSLKLSSVTAADTAVYYCARDENWDGAHFAYWGQGTLVTVSS

SEQ ID NO: 278 - protein, 119 aa, synthetic construct
  REGION 1..119: note = VH domain of A1,2X4 IgG histidine scanning variant #37
QVQLQESGPGLVKPSETLSLTCTVSGFSLTSYGVNWIRQPPGKGLEWIGVMWAGGSTNYNSALMSRLTISKDTSKNQFSLKLSSVTAADTAVYYCARDENWDGAWHAYWGQGTLVTVSS

SEQ ID NO: 279 - protein, 119 aa, synthetic construct
  REGION 1..119: note = VH domain of A1,2X4 IgG histidine scanning variant #38
QVQLQESGPGLVKPSETLSLTCTVSGFSLTSYGVNWIRQPPGKGLEWIGVMWAGGSTNYNSALMSRLTISKDTSKNQFSLKLSSVTAADTAVYYCARDENWDGAWFHYWGQGTLVTVSS

SEQ ID NO: 280 - protein, 119 aa, synthetic construct
  REGION 1..119: note = VH domain of A1,2X4 IgG histidine scanning variant #39
QVQLQESGPGLVKPSETLSLTCTVSGFSLTSYGVNWIRQPPGKGLEWIGVMWAGGSTNYNSALMSRLTISKDTSKNQFSLKLSSVTAADTAVYYCARDENWDGAWFAHWGQGTLVTVSS

SEQ ID NO: 281 - protein, 112 aa, synthetic construct
  REGION 1..112: note = VL domain of A1,2X4 IgG histidine scanning variant #1
DIVMTQSPDSLAVSLGERATINCHSSQSLLNSGTRKNYLAWYQQKPGQPPKLLISWASSRESGVPDRFSGSGSGTDFTLTISSLQAEDVAVYYCKQSYNLFTFGGGTKVEIK

SEQ ID NO: 282 - protein, 112 aa, synthetic construct
  REGION 1..112: note = VL domain of A1,2X4 IgG histidine scanning variant #2
DIVMTQSPDSLAVSLGERATINCKHSQSLLNSGTRKNYLAWYQQKPGQPPKLLISWASSRESGVPDRFSGSGSGTDFTLTISSLQAEDVAVYYCKQSYNLFTFGGGTKVEIK

SEQ ID NO: 283 - protein, 112 aa, synthetic construct
  REGION 1..112: note = VL domain of A1,2X4 IgG histidine scanning variant #3
DIVMTQSPDSLAVSLGERATINCKSHQSLLNSGTRKNYLAWYQQKPGQPPKLLISWASSRESGVPDRFSGSGSGTDFTLTISSLQAEDVAVYYCKQSYNLFTFGGGTKVEIK

SEQ ID NO: 284 - protein, 112 aa, synthetic construct
  REGION 1..112: note = VL domain of A1,2X4 IgG histidine scanning variant #4
DIVMTQSPDSLAVSLGERATINCKSSHSLLNSGTRKNYLAWYQQKPGQPPKLLISWASSRESGVPDRFSGSGSGTDFTLTISSLQAEDVAVYYCKQSYNLFTFGGGTKVEIK

SEQ ID NO: 285 - protein, 112 aa, synthetic construct
  REGION 1..112: note = VL domain of A1,2X4 IgG histidine scanning variant #5
DIVMTQSPDSLAVSLGERATINCKSSQHLLNSGTRKNYLAWYQQKPGQPPKLLISWASSRESGVPDRFSGSGSGTDFTLTISSLQAEDVAVYYCKQSYNLFTFGGGTKVEIK

SEQ ID NO: 286 - protein, 112 aa, synthetic construct
  REGION 1..112: note = VL domain of A1,2X4 IgG histidine scanning variant #6
DIVMTQSPDSLAVSLGERATINCKSSQSHLNSGTRKNYLAWYQQKPGQPPKLLISWASSRESGVPDRFSGSGSGTDFTLTISSLQAEDVAVYYCKQSYNLFTFGGGTKVEIK

SEQ ID NO: 287 - protein, 112 aa, synthetic construct
  REGION 1..112: note = VL domain of A1,2X4 IgG histidine scanning variant #7
DIVMTQSPDSLAVSLGERATINCKSSQSLHNSGTRKNYLAWYQQKPGQPPKLLISWASSRESGVPDRFSGSGSGTDFTLTISSLQAEDVAVYYCKQSYNLFTFGGGTKVEIK

SEQ ID NO: 288 - protein, 112 aa, synthetic construct
  REGION 1..112: note = VL domain of A1,2X4 IgG histidine scanning variant #8
DIVMTQSPDSLAVSLGERATINCKSSQSLLHSGTRKNYLAWYQQKPGQPPKLLISWASSRESGVPDRFSGSGSGTDFTLTISSLQAEDVAVYYCKQSYNLFTFGGGTKVEIK

SEQ ID NO: 289 - protein, 112 aa, synthetic construct
  REGION 1..112: note = VL domain of A1,2X4 IgG histidine scanning variant #9
DIVMTQSPDSLAVSLGERATINCKSSQSLLNHGTRKNYLAWYQQKPGQPPKLLISWASSRESGVPDRFSGSGSGTDFTLTISSLQAEDVAVYYCKQSYNLFTFGGGTKVEIK

SEQ ID NO: 290 - protein, 112 aa, synthetic construct
  REGION 1..112: note = VL domain of A1,2X4 IgG histidine scanning variant #10
DIVMTQSPDSLAVSLGERATINCKSSQSLLNSHTRKNYLAWYQQKPGQPPKLLISWASSRESGVPDRFSGSGSGTDFTLTISSLQAEDVAVYYCKQSYNLFTFGGGTKVEIK

SEQ ID NO: 291 - protein, 112 aa, synthetic construct
  REGION 1..112: note = VL domain of A1,2X4 IgG histidine scanning variant #11
DIVMTQSPDSLAVSLGERATINCKSSQSLLNSGHRKNYLAWYQQKPGQPPKLLISWASSRESGVPDRFSGSGSGTDFTLTISSLQAEDVAVYYCKQSYNLFTFGGGTKVEIK

SEQ ID NO: 292 - protein, 112 aa, synthetic construct
  REGION 1..112: note = VL domain of A1,2X4 IgG histidine scanning variant #12
DIVMTQSPDSLAVSLGERATINCKSSQSLLNSGTHKNYLAWYQQKPGQPPKLLISWASSRESGVPDRFSGSGSGTDFTLTISSLQAEDVAVYYCKQSYNLFTFGGGTKVEIK

SEQ ID NO: 293 - protein, 112 aa, synthetic construct
  REGION 1..112: note = VL domain of A1,2X4 IgG histidine scanning variant #13
DIVMTQSPDSLAVSLGERATINCKSSQSLLNSGTRHNYLAWYQQKPGQPPKLLISWASSRESGVPDRFSGSGSGTDFTLTISSLQAEDVAVYYCKQSYNLFTFGGGTKVEIK

SEQ ID NO: 294 - protein, 112 aa, synthetic construct
  REGION 1..112: note = VL domain of A1,2X4 IgG histidine scanning variant #14
DIVMTQSPDSLAVSLGERATINCKSSQSLLNSGTRKHYLAWYQQKPGQPPKLLISWASSRESGVPDRFSGSGSGTDFTLTISSLQAEDVAVYYCKQSYNLFTFGGGTKVEIK

SEQ ID NO: 295 - protein, 112 aa, synthetic construct
  REGION 1..112: note = VL domain of A1,2X4 IgG histidine scanning variant #15
DIVMTQSPDSLAVSLGERATINCKSSQSLLNSGTRKNHLAWYQQKPGQPPKLLISWASSRESGVPDRFSGSGSGTDFTLTISSLQAEDVAVYYCKQSYNLFTFGGGTKVEIK

SEQ ID NO: 296 - protein, 112 aa, synthetic construct
  REGION 1..112: note = VL domain of A1,2X4 IgG histidine scanning variant #16
DIVMTQSPDSLAVSLGERATINCKSSQSLLNSGTRKNYHAWYQQKPGQPPKLLISWASSRESGVPDRFSGSGSGTDFTLTISSLQAEDVAVYYCKQSYNLFTFGGGTKVEIK

SEQ ID NO: 297 - protein, 112 aa, synthetic construct
  REGION 1..112: note = VL domain of A1,2X4 IgG histidine scanning variant #17
DIVMTQSPDSLAVSLGERATINCKSSQSLLNSGTRKNYLHWYQQKPGQPPKLLISWASSRESGVPDRFSGSGSGTDFTLTISSLQAEDVAVYYCKQSYNLFTFGGGTKVEIK

SEQ ID NO: 298 - protein, 112 aa, synthetic construct
  REGION 1..112: note = VL domain of A1,2X4 IgG histidine scanning variant #18
DIVMTQSPDSLAVSLGERATINCKSSQSLLNSGTRKNYLAWYQQKPGQPPKLLISHASSRESGVPDRFSGSGSGTDFTLTISSLQAEDVAVYYCKQSYNLFTFGGGTKVEIK

SEQ ID NO: 299 - protein, 112 aa, synthetic construct
  REGION 1..112: note = VL domain of A1,2X4 IgG histidine scanning variant #19
DIVMTQSPDSLAVSLGERATINCKSSQSLLNSGTRKNYLAWYQQKPGQPPKLLISWHSSRESGVPDRFSGSGSGTDFTLTISSLQAEDVAVYYCKQSYNLFTFGGGTKVEIK

SEQ ID NO: 300 - protein, 112 aa, synthetic construct
  REGION 1..112: note = VL domain of A1,2X4 IgG histidine scanning variant #20
DIVMTQSPDSLAVSLGERATINCKSSQSLLNSGTRKNYLAWYQQKPGQPPKLLISWAHSRESGVPDRFSGSGSGTDFTLTISSLQAEDVAVYYCKQSYNLFTFGGGTKVEIK

SEQ ID NO: 301 - protein, 112 aa, synthetic construct
  REGION 1..112: note = VL domain of A1,2X4 IgG histidine scanning variant #21
DIVMTQSPDSLAVSLGERATINCKSSQSLLNSGTRKNYLAWYQQKPGQPPKLLISWASHRESGVPDRFSGSGSGTDFTLTISSLQAEDVAVYYCKQSYNLFTFGGGTKVEIK

SEQ ID NO: 302 - protein, 112 aa, synthetic construct
  REGION 1..112: note = VL domain of A1,2X4 IgG histidine scanning variant #22
DIVMTQSPDSLAVSLGERATINCKSSQSLLNSGTRKNYLAWYQQKPGQPPKLLISWASSHESGVPDRFSGSGSGTDFTLTISSLQAEDVAVYYCKQSYNLFTFGGGTKVEIK

SEQ ID NO: 303 - protein, 112 aa, synthetic construct
  REGION 1..112: note = VL domain of A1,2X4 IgG histidine scanning variant #23
DIVMTQSPDSLAVSLGERATINCKSSQSLLNSGTRKNYLAWYQQKPGQPPKLLISWASSRHSGVPDRFSGSGSGTDFTLTISSLQAEDVAVYYCKQSYNLFTFGGGTKVEIK

SEQ ID NO: 304 - protein, 112 aa, synthetic construct
  REGION 1..112: note = VL domain of A1,2X4 IgG histidine scanning variant #24
DIVMTQSPDSLAVSLGERATINCKSSQSLLNSGTRKNYLAWYQQKPGQPPKLLISWASSREHGVPDRFSGSGSGTDFTLTISSLQAEDVAVYYCKQSYNLFTFGGGTKVEIK

SEQ ID NO: 305 - protein, 112 aa, synthetic construct
  REGION 1..112: note = VL domain of A1,2X4 IgG histidine scanning variant #25
DIVMTQSPDSLAVSLGERATINCKSSQSLLNSGTRKNYLAWYQQKPGQPPKLLISWASSRESGVPDRFSGSGSGTDFTLTISSLQAEDVAVYYCHQSYNLFTFGGGTKVEIK

SEQ ID NO: 306 - protein, 112 aa, synthetic construct
  REGION 1..112: note = VL domain of A1,2X4 IgG histidine scanning variant #26
DIVMTQSPDSLAVSLGERATINCKSSQSLLNSGTRKNYLAWYQQKPGQPPKLLISWASSRESGVPDRFSGSGSGTDFTLTISSLQAEDVAVYYCKHSYNLFTFGGGTKVEIK

SEQ ID NO: 307 - protein, 112 aa, synthetic construct
  REGION 1..112: note = VL domain of A1,2X4 IgG histidine scanning variant #27
DIVMTQSPDSLAVSLGERATINCKSSQSLLNSGTRKNYLAWYQQKPGQPPKLLISWASSRESGVPDRFSGSGSGTDFTLTISSLQAEDVAVYYCKQHYNLFTFGGGTKVEIK

SEQ ID NO: 308 - protein, 112 aa, synthetic construct
  REGION 1..112: note = VL domain of A1,2X4 IgG histidine scanning variant #28
DIVMTQSPDSLAVSLGERATINCKSSQSLLNSGTRKNYLAWYQQKPGQPPKLLISWASSRESGVPDRFSGSGSGTDFTLTISSLQAEDVAVYYCKQSHNLFTFGGGTKVEIK

SEQ ID NO: 309 - protein, 112 aa, synthetic construct
  REGION 1..112: note = VL domain of A1,2X4 IgG histidine scanning variant #29
DIVMTQSPDSLAVSLGERATINCKSSQSLLNSGTRKNYLAWYQQKPGQPPKLLISWASSRESGVPDRFSGSGSGTDFTLTISSLQAEDVAVYYCKQSYHLFTFGGGTKVEIK

SEQ ID NO: 310 - protein, 112 aa, synthetic construct
  REGION 1..112: note = VL domain of A1,2X4 IgG histidine scanning variant #30
DIVMTQSPDSLAVSLGERATINCKSSQSLLNSGTRKNYLAWYQQKPGQPPKLLISWASSRESGVPDRFSGSGSGTDFTLTISSLQAEDVAVYYCKQSYNHFTFGGGTKVEIK

SEQ ID NO: 311 - protein, 112 aa, synthetic construct
  REGION 1..112: note = VL domain of A1,2X4 IgG histidine scanning variant #31
DIVMTQSPDSLAVSLGERATINCKSSQSLLNSGTRKNYLAWYQQKPGQPPKLLISWASSRESGVPDRFSGSGSGTDFTLTISSLQAEDVAVYYCKQSYNLHTFGGGTKVEIK

SEQ ID NO: 312 - protein, 112 aa, synthetic construct
  REGION 1..112: note = VL domain of A1,2X4 IgG histidine scanning variant #32
DIVMTQSPDSLAVSLGERATINCKSSQSLLNSGTRKNYLAWYQQKPGQPPKLLISWASSRESGVPDRFSGSGSGTDFTLTISSLQAEDVAVYYCKQSYNLFHFGGGTKVEIK

SEQ ID NO: 313 - protein, 121 aa, synthetic construct
  REGION 1..121: note = VH domain of Sacituzumab IgG with double histidine substitution
QVQLQQSGSELKKPGASVKVSCKASGYTFTNHHMNWVKQAPGQGLKWMGWINTYTGEPTYTDDFKGRFAFSLDTSVSTAYLQISSLKADDTAVYFCARGGFGSSYWYFDVWGQGSLVTVSS

SEQ ID NO: 314 - protein, 121 aa, synthetic construct
  REGION 1..121: note = VH domain of Sacituzumab IgG with double histidine substitution
QVQLQQSGSELKKPGASVKVSCKASGYTFTNHGMNWVKQAPGQGLKWMGWINTYTGEPTHTDDFKGRFAFSLDTSVSTAYLQISSLKADDTAVYFCARGGFGSSYWYFDVWGQGSLVTVSS

SEQ ID NO: 315 - protein, 121 aa, synthetic construct
  REGION 1..121: note = VH domain of Sacituzumab IgG with double histidine substitution
QVQLQQSGSELKKPGASVKVSCKASGYTFTNHGMNWVKQAPGQGLKWMGWINTYTGEPTYTHDFKGRFAFSLDTSVSTAYLQISSLKADDTAVYFCARGGFGSSYWYFDVWGQGSLVTVSS

SEQ ID NO: 316 - protein, 121 aa, synthetic construct
  REGION 1..121: note = VH domain of Sacituzumab IgG with double histidine substitution
QVQLQQSGSELKKPGASVKVSCKASGYTFTNHGMNWVKQAPGQGLKWMGWINTYTGEPTYTDHFKGRFAFSLDTSVSTAYLQISSLKADDTAVYFCARGGFGSSYWYFDVWGQGSLVTVSS

SEQ ID NO: 317 - protein, 121 aa, synthetic construct
  REGION 1..121: note = VH domain of Sacituzumab IgG with double histidine substitution
QVQLQQSGSELKKPGASVKVSCKASGYTFTNHGMNWVKQAPGQGLKWMGWINTYTGEPTYTDDHKGRFAFSLDTSVSTAYLQISSLKADDTAVYFCARGGFGSSYWYFDVWGQGSLVTVSS

SEQ ID NO: 318 - protein, 121 aa, synthetic construct
  REGION 1..121: note = VH domain of Sacituzumab IgG with double histidine substitution
QVQLQQSGSELKKPGASVKVSCKASGYTFTNHGMNWVKQAPGQGLKWMGWINTYTGEPTYTDDFHGRFAFSLDTSVSTAYLQISSLKADDTAVYFCARGGFGSSYWYFDVWGQGSLVTVSS

SEQ ID NO: 319 - protein, 121 aa, synthetic construct
  REGION 1..121: note = VH domain of Sacituzumab IgG with double histidine substitution
QVQLQQSGSELKKPGASVKVSCKASGYTFTNHGMNWVKQAPGQGLKWMGWINTYTGEPTYTDDFKHRFAFSLDTSVSTAYLQISSLKADDTAVYFCARGGFGSSYWYFDVWGQGSLVTVSS

SEQ ID NO: 320 - protein, 121 aa, synthetic construct
  REGION 1..121: note = VH domain of Sacituzumab IgG with double histidine substitution
QVQLQQSGSELKKPGASVKVSCKASGYTFTNYHMNWVKQAPGQGLKWMGWINTYTGEPTHTDDFKGRFAFSLDTSVSTAYLQISSLKADDTAVYFCARGGFGSSYWYFDVWGQGSLVTVSS

SEQ ID NO: 321 - protein, 121 aa, synthetic construct
  REGION 1..121: note = VH domain of Sacituzumab IgG with double histidine substitution
QVQLQQSGSELKKPGASVKVSCKASGYTFTNYHMNWVKQAPGQGLKWMGWINTYTGEPTYTHDFKGRFAFSLDTSVSTAYLQISSLKADDTAVYFCARGGFGSSYWYFDVWGQGSLVTVSS

SEQ ID NO: 322 - protein, 121 aa, synthetic construct
  REGION 1..121: note = VH domain of Sacituzumab IgG with double histidine substitution
QVQLQQSGSELKKPGASVKVSCKASGYTFTNYHMNWVKQAPGQGLKWMGWINTYTGEPTYTDHFKGRFAFSLDTSVSTAYLQISSLKADDTAVYFCARGGFGSSYWYFDVWGQGSLVTVSS

SEQ ID NO: 323 - protein, 121 aa, synthetic construct
  REGION 1..121: note = VH domain of Sacituzumab IgG with double histidine substitution
QVQLQQSGSELKKPGASVKVSCKASGYTFTNYHMNWVKQAPGQGLKWMGWINTYTGEPTYTDDHKGRFAFSLDTSVSTAYLQISSLKADDTAVYFCARGGFGSSYWYFDVWGQGSLVTVSS

SEQ ID NO: 324 - protein, 121 aa, synthetic construct
  REGION 1..121: note = VH domain of Sacituzumab IgG with double histidine substitution
QVQLQQSGSELKKPGASVKVSCKASGYTFTNYHMNWVKQAPGQGLKWMGWINTYTGEPTYTDDFHGRFAFSLDTSVSTAYLQISSLKADDTAVYFCARGGFGSSYWYFDVWGQGSLVTVSS

SEQ ID NO: 325 - protein, 121 aa, synthetic construct
  REGION 1..121: note = VH domain of Sacituzumab IgG with double histidine substitution
QVQLQQSGSELKKPGASVKVSCKASGYTFTNYHMNWVKQAPGQGLKWMGWINTYTGEPTYTDDFKHRFAFSLDTSVSTAYLQISSLKADDTAVYFCARGGFGSSYWYFDVWGQGSLVTVSS

SEQ ID NO: 326 - protein, 121 aa, synthetic construct
  REGION 1..121: note = VH domain of Sacituzumab IgG with double histidine substitution
QVQLQQSGSELKKPGASVKVSCKASGYTFTNYGMNWVKQAPGQGLKWMGWINTYTGEPTHTHDFKGRFAFSLDTSVSTAYLQISSLKADDTAVYFCARGGFGSSYWYFDVWGQGSLVTVSS

SEQ ID NO: 327 - protein, 121 aa, synthetic construct
  REGION 1..121: note = VH domain of Sacituzumab IgG with double histidine substitution
QVQLQQSGSELKKPGASVKVSCKASGYTFTNYGMNWVKQAPGQGLKWMGWINTYTGEPTHTDHFKGRFAFSLDTSVSTAYLQISSLKADDTAVYFCARGGFGSSYWYFDVWGQGSLVTVSS

SEQ ID NO: 328 - protein, 121 aa, synthetic construct
  REGION 1..121: note = VH domain of Sacituzumab IgG with double histidine substitution
QVQLQQSGSELKKPGASVKVSCKASGYTFTNYGMNWVKQAPGQGLKWMGWINTYTGEPTHTDDHKGRFAFSLDTSVSTAYLQISSLKADDTAVYFCARGGFGSSYWYFDVWGQGSLVTVSS

SEQ ID NO: 329 - protein, 121 aa, synthetic construct
  REGION 1..121: note = VH domain of Sacituzumab IgG with double histidine substitution
QVQLQQSGSELKKPGASVKVSCKASGYTFTNYGMNWVKQAPGQGLKWMGWINTYTGEPTHTDDFHGRFAFSLDTSVSTAYLQISSLKADDTAVYFCARGGFGSSYWYFDVWGQGSLVTVSS

SEQ ID NO: 330 - protein, 121 aa, synthetic construct
  REGION 1..121: note = VH domain of Sacituzumab IgG with double histidine substitution
QVQLQQSGSELKKPGASVKVSCKASGYTFTNYGMNWVKQAPGQGLKWMGWINTYTGEPTHTDDFKHRFAFSLDTSVSTAYLQISSLKADDTAVYFCARGGFGSSYWYFDVWGQGSLVTVSS

SEQ ID NO: 331 - protein, 121 aa, synthetic construct
  REGION 1..121: note = VH domain of Sacituzumab IgG with double histidine substitution
QVQLQQSGSELKKPGASVKVSCKASGYTFTNYGMNWVKQAPGQGLKWMGWINTYTGEPTYTHHFKGRFAFSLDTSVSTAYLQISSLKADDTAVYFCARGGFGSSYWYFDVWGQGSLVTVSS

SEQ ID NO: 332 - protein, 121 aa, synthetic construct
  REGION 1..121: note = VH domain of Sacituzumab IgG with double histidine substitution
QVQLQQSGSELKKPGASVKVSCKASGYTFTNYGMNWVKQAPGQGLKWMGWINTYTGEPTYTHDHKGRFAFSLDTSVSTAYLQISSLKADDTAVYFCARGGFGSSYWYFDVWGQGSLVTVSS

SEQ ID NO: 333 - protein, 121 aa, synthetic construct
  REGION 1..121: note = VH domain of Sacituzumab IgG with double histidine substitution
QVQLQQSGSELKKPGASVKVSCKASGYTFTNYGMNWVKQAPGQGLKWMGWINTYTGEPTYTHDFHGRFAFSLDTSVSTAYLQISSLKADDTAVYFCARGGFGSSYWYFDVWGQGSLVTVSS

SEQ ID NO: 334 - protein, 121 aa, synthetic construct
  REGION 1..121: note = VH domain of Sacituzumab IgG with double histidine substitution
QVQLQQSGSELKKPGASVKVSCKASGYTFTNYGMNWVKQAPGQGLKWMGWINTYTGEPTYTHDFKHRFAFSLDTSVSTAYLQISSLKADDTAVYFCARGGFGSSYWYFDVWGQGSLVTVSS

SEQ ID NO: 335 - protein, 121 aa, synthetic construct
  REGION 1..121: note = VH domain of Sacituzumab IgG with double histidine substitution
QVQLQQSGSELKKPGASVKVSCKASGYTFTNYGMNWVKQAPGQGLKWMGWINTYTGEPTYTDHHKGRFAFSLDTSVSTAYLQISSLKADDTAVYFCARGGFGSSYWYFDVWGQGSLVTVSS

SEQ ID NO: 336 - protein, 121 aa, synthetic construct
  REGION 1..121: note = VH domain of Sacituzumab IgG with double histidine substitution
QVQLQQSGSELKKPGASVKVSCKASGYTFTNYGMNWVKQAPGQGLKWMGWINTYTGEPTYTDHFHGRFAFSLDTSVSTAYLQISSLKADDTAVYFCARGGFGSSYWYFDVWGQGSLVTVSS

SEQ ID NO: 337 - protein, 121 aa, synthetic construct
  REGION 1..121: note = VH domain of Sacituzumab IgG with double histidine substitution
QVQLQQSGSELKKPGASVKVSCKASGYTFTNYGMNWVKQAPGQGLKWMGWINTYTGEPTYTDHFKHRFAFSLDTSVSTAYLQISSLKADDTAVYFCARGGFGSSYWYFDVWGQGSLVTVSS

SEQ ID NO: 338 - protein, 121 aa, synthetic construct
  REGION 1..121: note = VH domain of Sacituzumab IgG with double histidine substitution
QVQLQQSGSELKKPGASVKVSCKASGYTFTNYGMNWVKQAPGQGLKWMGWINTYTGEPTYTDDHHGRFAFSLDTSVSTAYLQISSLKADDTAVYFCARGGFGSSYWYFDVWGQGSLVTVSS

SEQ ID NO: 339 - protein, 121 aa, synthetic construct
  REGION 1..121: note = VH domain of Sacituzumab IgG with double histidine substitution
QVQLQQSGSELKKPGASVKVSCKASGYTFTNYGMNWVKQAPGQGLKWMGWINTYTGEPTYTDDHKHRFAFSLDTSVSTAYLQISSLKADDTAVYFCARGGFGSSYWYFDVWGQGSLVTVSS

SEQ ID NO: 340 - protein, 121 aa, synthetic construct
  REGION 1..121: note = VH domain of Sacituzumab IgG with double histidine substitution
QVQLQQSGSELKKPGASVKVSCKASGYTFTNYGMNWVKQAPGQGLKWMGWINTYTGEPTYTDDFHHRFAFSLDTSVSTAYLQISSLKADDTAVYFCARGGFGSSYWYFDVWGQGSLVTVSS

SEQ ID NO: 341 - protein, 121 aa, synthetic construct
  REGION 1..121: note = VH domain of Sacituzumab IgG with triple histidine substitution
QVQLQQSGSELKKPGASVKVSCKASGYTFTNYHMNWVKQAPGQGLKWMGWINTYTGEPTHTDDHKGRFAFSLDTSVSTAYLQISSLKADDTAVYFCARGGFGSSYWYFDVWGQGSLVTVSS

SEQ ID NO: 342 - protein, 121 aa, synthetic construct
  REGION 1..121: note = VH domain of Sacituzumab IgG with triple histidine substitution
QVQLQQSGSELKKPGASVKVSCKASGYTFTNYHMNWVKQAPGQGLKWMGWINTYTGEPTHTDDFHGRFAFSLDTSVSTAYLQISSLKADDTAVYFCARGGFGSSYWYFDVWGQGSLVTVSS

SEQ ID NO: 343 - protein, 121 aa, synthetic construct
  REGION 1..121: note = VH domain of Sacituzumab IgG with triple histidine substitution
QVQLQQSGSELKKPGASVKVSCKASGYTFTNYHMNWVKQAPGQGLKWMGWINTYTGEPTHTDDFKHRFAFSLDTSVSTAYLQISSLKADDTAVYFCARGGFGSSYWYFDVWGQGSLVTVSS

SEQ ID NO: 344 - protein, 121 aa, synthetic construct
  REGION 1..121: note = VH domain of Sacituzumab IgG with triple histidine substitution
QVQLQQSGSELKKPGASVKVSCKASGYTFTNYHMNWVKQAPGQGLKWMGWINTYTGEPTYTDDHHGRFAFSLDTSVSTAYLQISSLKADDTAVYFCARGGFGSSYWYFDVWGQGSLVTVSS

SEQ ID NO: 345 - protein, 121 aa, synthetic construct
  REGION 1..121: note = VH domain of Sacituzumab IgG with triple histidine substitution
QVQLQQSGSELKKPGASVKVSCKASGYTFTNYHMNWVKQAPGQGLKWMGWINTYTGEPTYTDDHKHRFAFSLDTSVSTAYLQISSLKADDTAVYFCARGGFGSSYWYFDVWGQGSLVTVSS

SEQ ID NO: 346 - protein, 121 aa, synthetic construct
  REGION 1..121: note = VH domain of Sacituzumab IgG with triple histidine substitution
QVQLQQSGSELKKPGASVKVSCKASGYTFTNYHMNWVKQAPGQGLKWMGWINTYTGEPTYTDDFHHRFAFSLDTSVSTAYLQISSLKADDTAVYFCARGGFGSSYWYFDVWGQGSLVTVSS

SEQ ID NO: 347 - protein, 121 aa, synthetic construct
  REGION 1..121: note = VH domain of Sacituzumab IgG with triple histidine substitution
QVQLQQSGSELKKPGASVKVSCKASGYTFTNYGMNWVKQAPGQGLKWMGWINTYTGEPTHTDDHHGRFAFSLDTSVSTAYLQISSLKADDTAVYFCARGGFGSSYWYFDVWGQGSLVTVSS

SEQ ID NO: 348 - protein, 121 aa, synthetic construct
  REGION 1..121: note = VH domain of Sacituzumab IgG with triple histidine substitution
QVQLQQSGSELKKPGASVKVSCKASGYTFTNYGMNWVKQAPGQGLKWMGWINTYTGEPTHTDDHKHRFAFSLDTSVSTAYLQISSLKADDTAVYFCARGGFGSSYWYFDVWGQGSLVTVSS

SEQ ID NO: 349 - protein, 121 aa, synthetic construct
  REGION 1..121: note = VH domain of Sacituzumab IgG with triple histidine substitution
QVQLQQSGSELKKPGASVKVSCKASGYTFTNYGMNWVKQAPGQGLKWMGWINTYTGEPTHTDDFHHRFAFSLDTSVSTAYLQISSLKADDTAVYFCARGGFGSSYWYFDVWGQGSLVTVSS

SEQ ID NO: 350 - protein, 121 aa, synthetic construct
  REGION 1..121: note = VH domain of Sacituzumab IgG with triple histidine substitution
QVQLQQSGSELKKPGASVKVSCKASGYTFTNYGMNWVKQAPGQGLKWMGWINTYTGEPTYTDDHHHRFAFSLDTSVSTAYLQISSLKADDTAVYFCARGGFGSSYWYFDVWGQGSLVTVSS

SEQ ID NO: 351 - protein, 107 aa, synthetic construct
  REGION 1..107: note = VL domain of Sacituzumab IgG with double histidine substitution
DIQLTQSPSSLSASVGDRVSITCKASQDVHIAHAWYQQKPGKAPKLLIYSASYRYTGVPDRFSGSGSGTDFTLTISSLQPEDFAVYYCQQHYITPLTFGAGTKVEIK

SEQ ID NO: 352 - protein, 107 aa, synthetic construct
  REGION 1..107: note = VL domain of Sacituzumab IgG with double histidine substitution
DIQLTQSPSSLSASVGDRVSITCKASQDVHIAVAWYQQKPGKAPKLLIYSHSYRYTGVPDRFSGSGSGTDFTLTISSLQPEDFAVYYCQQHYITPLTFGAGTKVEIK

SEQ ID NO: 353 - protein, 107 aa, synthetic construct
  REGION 1..107: note = VL domain of Sacituzumab IgG with double histidine substitution
DIQLTQSPSSLSASVGDRVSITCKASQDVHIAVAWYQQKPGKAPKLLIYSAHYRYTGVPDRFSGSGSGTDFTLTISSLQPEDFAVYYCQQHYITPLTFGAGTKVEIK

SEQ ID NO: 354 - protein, 107 aa, synthetic construct
  REGION 1..107: note = VL domain of Sacituzumab IgG with double histidine substitution
DIQLTQSPSSLSASVGDRVSITCKASQDVHIAVAWYQQKPGKAPKLLIYSASHRYTGVPDRFSGSGSGTDFTLTISSLQPEDFAVYYCQQHYITPLTFGAGTKVEIK

SEQ ID NO: 355 - protein, 107 aa, synthetic construct
  REGION 1..107: note = VL domain of Sacituzumab IgG with double histidine substitution
DIQLTQSPSSLSASVGDRVSITCKASQDVHIAVAWYQQKPGKAPKLLIYSASYRYTGVPDRFSGSGSGTDFTLTISSLQPEDFAVYYCHQHYITPLTFGAGTKVEIK

SEQ ID NO: 356 - protein, 107 aa, synthetic construct
  REGION 1..107: note = VL domain of Sacituzumab IgG with double histidine substitution
DIQLTQSPSSLSASVGDRVSITCKASQDVHIAVAWYQQKPGKAPKLLIYSASYRYTGVPDRFSGSGSGTDFTLTISSLQPEDFAVYYCQQAYITPLTFGAGTKVEIK

SEQ ID NO: 357 - protein, 107 aa, synthetic construct
  REGION 1..107: note = VL domain of Sacituzumab IgG with double histidine substitution
DIQLTQSPSSLSASVGDRVSITCKASQDVHIAVAWYQQKPGKAPKLLIYSASYRYTGVPDRFSGSGSGTDFTLTISSLQPEDFAVYYCQQHHITPLTFGAGTKVEIK

SEQ ID NO: 358 - protein, 107 aa, synthetic construct
  REGION 1..107: note = VL domain of Sacituzumab IgG with double histidine substitution
DIQLTQSPSSLSASVGDRVSITCKASQDVHIAVAWYQQKPGKAPKLLIYSASYRYTGVPDRFSGSGSGTDFTLTISSLQPEDFAVYYCQQHYHTPLTFGAGTKVEIK

SEQ ID NO: 359 - protein, 107 aa, synthetic construct
  REGION 1..107: note = VL domain of Sacituzumab IgG with double histidine substitution
DIQLTQSPSSLSASVGDRVSITCKASQDVSIAHAWYQQKPGKAPKLLIYSHSYRYTGVPDRFSGSGSGTDFTLTISSLQPEDFAVYYCQQHYITPLTFGAGTKVEIK

SEQ ID NO: 360 - protein, 107 aa, synthetic construct
  REGION 1..107: note = VL domain of Sacituzumab IgG with double histidine substitution
DIQLTQSPSSLSASVGDRVSITCKASQDVSIAHAWYQQKPGKAPKLLIYSAHYRYTGVPDRFSGSGSGTDFTLTISSLQPEDFAVYYCQQHYITPLTFGAGTKVEIK

SEQ ID NO: 361 - protein, 107 aa, synthetic construct
  REGION 1..107: note = VL domain of Sacituzumab IgG with double histidine substitution
DIQLTQSPSSLSASVGDRVSITCKASQDVSIAHAWYQQKPGKAPKLLIYSASHRYTGVPDRFSGSGSGTDFTLTISSLQPEDFAVYYCQQHYITPLTFGAGTKVEIK

SEQ ID NO: 362 - protein, 107 aa, synthetic construct
  REGION 1..107: note = VL domain of Sacituzumab IgG with double histidine substitution
DIQLTQSPSSLSASVGDRVSITCKASQDVSIAHAWYQQKPGKAPKLLIYSASYRYTGVPDRFSGSGSGTDFTLTISSLQPEDFAVYYCHQHYITPLTFGAGTKVEIK

SEQ ID NO: 363 - protein, 107 aa, synthetic construct
  REGION 1..107: note = VL domain of Sacituzumab IgG with double histidine substitution
DIQLTQSPSSLSASVGDRVSITCKASQDVSIAHAWYQQKPGKAPKLLIYSASYRYTGVPDRFSGSGSGTDFTLTISSLQPEDFAVYYCQQAYITPLTFGAGTKVEIK

SEQ ID NO: 364 - protein, 107 aa, synthetic construct
  REGION 1..107: note = VL domain of Sacituzumab IgG with double histidine substitution
DIQLTQSPSSLSASVGDRVSITCKASQDVSIAHAWYQQKPGKAPKLLIYSASYRYTGVPDRFSGSGSGTDFTLTISSLQPEDFAVYYCQQHHITPLTFGAGTKVEIK

SEQ ID NO: 365 - protein, 107 aa, synthetic construct
  REGION 1..107: note = VL domain of Sacituzumab IgG with double histidine substitution
DIQLTQSPSSLSASVGDRVSITCKASQDVSIAHAWYQQKPGKAPKLLIYSASYRYTGVPDRFSGSGSGTDFTLTISSLQPEDFAVYYCQQHYHTPLTFGAGTKVEIK

SEQ ID NO: 366 - protein, 107 aa, synthetic construct
  REGION 1..107: note = VL domain of Sacituzumab IgG with double histidine substitution
DIQLTQSPSSLSASVGDRVSITCKASQDVSIAVAWYQQKPGKAPKLLIYSHHYRYTGVPDRFSGSGSGTDFTLTISSLQPEDFAVYYCQQHYITPLTFGAGTKVEIK

SEQ ID NO: 367 - protein, 107 aa, synthetic construct
  REGION 1..107: note = VL domain of Sacituzumab IgG with double histidine substitution
DIQLTQSPSSLSASVGDRVSITCKASQDVSIAVAWYQQKPGKAPKLLIYSHSHRYTGVPDRFSGSGSGTDFTLTISSLQPEDFAVYYCQQHYITPLTFGAGTKVEIK

SEQ ID NO: 368 - protein, 107 aa, synthetic construct
  REGION 1..107: note = VL domain of Sacituzumab IgG with double histidine substitution
DIQLTQSPSSLSASVGDRVSITCKASQDVSIAVAWYQQKPGKAPKLLIYSHSYRYTGVPDRFSGSGSGTDFTLTISSLQPEDFAVYYCHQHYITPLTFGAGTKVEIK

SEQ ID NO: 369 - protein, 107 aa, synthetic construct
  REGION 1..107: note = VL domain of Sacituzumab IgG with double histidine substitution
DIQLTQSPSSLSASVGDRVSITCKASQDVSIAVAWYQQKPGKAPKLLIYSHSYRYTGVPDRFSGSGSGTDFTLTISSLQPEDFAVYYCQQAYITPLTFGAGTKVEIK

SEQ ID NO: 370 - protein, 107 aa, synthetic construct
  REGION 1..107: note = VL domain of Sacituzumab IgG with double histidine substitution
DIQLTQSPSSLSASVGDRVSITCKASQDVSIAVAWYQQKPGKAPKLLIYSHSYRYTGVPDRFSGSGSGTDFTLTISSLQPEDFAVYYCQQHHITPLTFGAGTKVEIK

SEQ ID NO: 371 - protein, 107 aa, synthetic construct
  REGION 1..107: note = VL domain of Sacituzumab IgG with double histidine substitution
DIQLTQSPSSLSASVGDRVSITCKASQDVSIAVAWYQQKPGKAPKLLIYSHSYRYTGVPDRFSGSGSGTDFTLTISSLQPEDFAVYYCQQHYHTPLTFGAGTKVEIK

SEQ ID NO: 372 - protein, 107 aa, synthetic construct
  REGION 1..107: note = VL domain of Sacituzumab IgG with double histidine substitution
DIQLTQSPSSLSASVGDRVSITCKASQDVSIAVAWYQQKPGKAPKLLIYSAHHRYTGVPDRFSGSGSGTDFTLTISSLQPEDFAVYYCQQHYITPLTFGAGTKVEIK

SEQ ID NO: 373 - protein, 107 aa, synthetic construct
  REGION 1..107: note = VL domain of Sacituzumab IgG with double histidine substitution
DIQLTQSPSSLSASVGDRVSITCKASQDVSIAVAWYQQKPGKAPKLLIYSAHYRYTGVPDRFSGSGSGTDFTLTISSLQPEDFAVYYCHQHYITPLTFGAGTKVEIK

SEQ ID NO: 374 - protein, 107 aa, synthetic construct
  REGION 1..107: note = VL domain of Sacituzumab IgG with double histidine substitution
DIQLTQSPSSLSASVGDRVSITCKASQDVSIAVAWYQQKPGKAPKLLIYSAHYRYTGVPDRFSGSGSGTDFTLTISSLQPEDFAVYYCQQAYITPLTFGAGTKVEIK

SEQ ID NO: 375 - protein, 107 aa, synthetic construct
  REGION 1..107: note = VL domain of Sacituzumab IgG with double histidine substitution
DIQLTQSPSSLSASVGDRVSITCKASQDVSIAVAWYQQKPGKAPKLLIYSAHYRYTGVPDRFSGSGSGTDFTLTISSLQPEDFAVYYCQQHHITPLTFGAGTKVEIK

SEQ ID NO: 376 - protein, 107 aa, synthetic construct
  REGION 1..107: note = VL domain of Sacituzumab IgG with double histidine substitution
DIQLTQSPSSLSASVGDRVSITCKASQDVSIAVAWYQQKPGKAPKLLIYSAHYRYTGVPDRFSGSGSGTDFTLTISSLQPEDFAVYYCQQHYHTPLTFGAGTKVEIK

SEQ ID NO: 377 - protein, 107 aa, synthetic construct
  REGION 1..107: note = VL domain of Sacituzumab IgG with double histidine substitution
DIQLTQSPSSLSASVGDRVSITCKASQDVSIAVAWYQQKPGKAPKLLIYSASHRYTGVPDRFSGSGSGTDFTLTISSLQPEDFAVYYCHQHYITPLTFGAGTKVEIK

SEQ ID NO: 378 - protein, 107 aa, synthetic construct
  REGION 1..107: note = VL domain of Sacituzumab IgG with double histidine substitution
DIQLTQSPSSLSASVGDRVSITCKASQDVSIAVAWYQQKPGKAPKLLIYSASHRYTGVPDRFSGSGSGTDFTLTISSLQPEDFAVYYCQQAYITPLTFGAGTKVEIK

SEQ ID NO: 379 - protein, 107 aa, synthetic construct
  REGION 1..107: note = VL domain of Sacituzumab IgG with double histidine substitution
DIQLTQSPSSLSASVGDRVSITCKASQDVSIAVAWYQQKPGKAPKLLIYSASHRYTGVPDRFSGSGSGTDFTLTISSLQPEDFAVYYCQQHHITPLTFGAGTKVEIK

SEQ ID NO: 380 - protein, 107 aa, synthetic construct
  REGION 1..107: note = VL domain of Sacituzumab IgG with double histidine substitution
DIQLTQSPSSLSASVGDRVSITCKASQDVSIAVAWYQQKPGKAPKLLIYSASHRYTGVPDRFSGSGSGTDFTLTISSLQPEDFAVYYCQQHYHTPLTFGAGTKVEIK

SEQ ID NO: 381 - protein, 107 aa, synthetic construct
  REGION 1..107: note = VL domain of Sacituzumab IgG with double histidine substitution
DIQLTQSPSSLSASVGDRVSITCKASQDVSIAVAWYQQKPGKAPKLLIYSASYRYTGVPDRFSGSGSGTDFTLTISSLQPEDFAVYYCHQAYITPLTFGAGTKVEIK

SEQ ID NO: 382 - protein, 107 aa, synthetic construct
  REGION 1..107: note = VL domain of Sacituzumab IgG with double histidine substitution
DIQLTQSPSSLSASVGDRVSITCKASQDVSIAVAWYQQKPGKAPKLLIYSASYRYTGVPDRFSGSGSGTDFTLTISSLQPEDFAVYYCHQHHITPLTFGAGTKVEIK

SEQ ID NO: 383 - protein, 107 aa, synthetic construct
  REGION 1..107: note = VL domain of Sacituzumab IgG with double histidine substitution
DIQLTQSPSSLSASVGDRVSITCKASQDVSIAVAWYQQKPGKAPKLLIYSASYRYTGVPDRFSGSGSGTDFTLTISSLQPEDFAVYYCHQHYHTPLTFGAGTKVEIK

SEQ ID NO: 384 - protein, 107 aa, synthetic construct
  REGION 1..107: note = VL domain of Sacituzumab IgG with double histidine substitution
DIQLTQSPSSLSASVGDRVSITCKASQDVSIAVAWYQQKPGKAPKLLIYSASYRYTGVPDRFSGSGSGTDFTLTISSLQPEDFAVYYCQQAHITPLTFGAGTKVEIK

SEQ ID NO: 385 - protein, 107 aa, synthetic construct
  REGION 1..107: note = VL domain of Sacituzumab IgG with double histidine substitution
DIQLTQSPSSLSASVGDRVSITCKASQDVSIAVAWYQQKPGKAPKLLIYSASYRYTGVPDRFSGSGSGTDFTLTISSLQPEDFAVYYCQQAYHTPLTFGAGTKVEIK

SEQ ID NO: 386 - protein, 107 aa, synthetic construct
  REGION 1..107: note = VL domain of Sacituzumab IgG with double histidine substitution
DIQLTQSPSSLSASVGDRVSITCKASQDVSIAVAWYQQKPGKAPKLLIYSASYRYTGVPDRFSGSGSGTDFTLTISSLQPEDFAVYYCQQHHHTPLTFGAGTKVEIK

SEQ ID NO: 387 - protein, 107 aa, synthetic construct
  REGION 1..107: note = VL domain of Sacituzumab IgG with triple histidine substitution
DIQLTQSPSSLSASVGDRVSITCKASQDVHIAVAWYQQKPGKAPKLLIYSHSHRYTGVPDRFSGSGSGTDFTLTISSLQPEDFAVYYCQQHYITPLTFGAGTKVEIK

SEQ ID NO: 388 - protein, 107 aa, synthetic construct
  REGION 1..107: note = VL domain of Sacituzumab IgG with triple histidine substitution
DIQLTQSPSSLSASVGDRVSITCKASQDVHIAVAWYQQKPGKAPKLLIYSHSYRYTGVPDRFSGSGSGTDFTLTISSLQPEDFAVYYCHQHYITPLTFGAGTKVEIK

SEQ ID NO: 389 - protein, 107 aa, synthetic construct
  REGION 1..107: note = VL domain of Sacituzumab IgG with triple histidine substitution
DIQLTQSPSSLSASVGDRVSITCKASQDVHIAVAWYQQKPGKAPKLLIYSHSYRYTGVPDRFSGSGSGTDFTLTISSLQPEDFAVYYCQQHYHTPLTFGAGTKVEIK

SEQ ID NO: 390 - protein, 107 aa, synthetic construct
  REGION 1..107: note = VL domain of Sacituzumab IgG with triple histidine substitution
DIQLTQSPSSLSASVGDRVSITCKASQDVHIAVAWYQQKPGKAPKLLIYSASHRYTGVPDRFSGSGSGTDFTLTISSLQPEDFAVYYCHQHYITPLTFGAGTKVEIK

SEQ ID NO: 391 - protein, 107 aa, synthetic construct
  REGION 1..107: note = VL domain of Sacituzumab IgG with triple histidine substitution
DIQLTQSPSSLSASVGDRVSITCKASQDVHIAVAWYQQKPGKAPKLLIYSASHRYTGVPDRFSGSGSGTDFTLTISSLQPEDFAVYYCQQHYHTPLTFGAGTKVEIK

SEQ ID NO: 392 - protein, 107 aa, synthetic construct
  REGION 1..107: note = VL domain of Sacituzumab IgG with triple histidine substitution
DIQLTQSPSSLSASVGDRVSITCKASQDVHIAVAWYQQKPGKAPKLLIYSASYRYTGVPDRFSGSGSGTDFTLTISSLQPEDFAVYYCHQHYHTPLTFGAGTKVEIK

SEQ ID NO: 393 - protein, 107 aa, synthetic construct
  REGION 1..107: note = VL domain of Sacituzumab IgG with triple histidine substitution
DIQLTQSPSSLSASVGDRVSITCKASQDVSIAVAWYQQKPGKAPKLLIYSHSHRYTGVPDRFSGSGSGTDFTLTISSLQPEDFAVYYCHQHYITPLTFGAGTKVEIK

SEQ ID NO: 394 - protein, 107 aa, synthetic construct
  REGION 1..107: note = VL domain of Sacituzumab IgG with triple histidine substitution
DIQLTQSPSSLSASVGDRVSITCKASQDVSIAVAWYQQKPGKAPKLLIYSHSHRYTGVPDRFSGSGSGTDFTLTISSLQPEDFAVYYCQQHYHTPLTFGAGTKVEIK

SEQ ID NO: 395 - protein, 107 aa, synthetic construct
  REGION 1..107: note = VL domain of Sacituzumab IgG with triple histidine substitution
DIQLTQSPSSLSASVGDRVSITCKASQDVSIAVAWYQQKPGKAPKLLIYSHSYRYTGVPDRFSGSGSGTDFTLTISSLQPEDFAVYYCHQHYHTPLTFGAGTKVEIK

SEQ ID NO: 396 - protein, 107 aa, synthetic construct
  REGION 1..107: note = VL domain of Sacituzumab IgG with triple histidine substitution
DIQLTQSPSSLSASVGDRVSITCKASQDVSIAVAWYQQKPGKAPKLLIYSASHRYTGVPDRFSGSGSGTDFTLTISSLQPEDFAVYYCHQHYHTPLTFGAGTKVEIK

SEQ ID NO: 397 - protein, 121 aa, synthetic construct
  REGION 1..121: note = VH domain of Datopotamab IgG with double histidine substitution
QVQLVQSGAEVKKPGASVKVSCKASGYTFTTAGMQWVRQAPGQGLEWMGWINHHSGVPKYAEDFKGRVTISADTSTSTAYLQLSSLKSEDTAVYYCAHSGFGSSYWYFDVWGQGTLVTVSS

SEQ ID NO: 398 - protein, 121 aa, synthetic construct
  REGION 1..121: note = VH domain of Datopotamab IgG with double histidine substitution
QVQLVQSGAEVKKPGASVKVSCKASGYTFTTAGMQWVRQAPGQGLEWMGWINHHSGVPKYAEDFKGRVTISADTSTSTAYLQLSSLKSEDTAVYYCARSGHGSSYWYFDVWGQGTLVTVSS

SEQ ID NO: 399 - protein, 121 aa, synthetic construct
  REGION 1..121: note = VH domain of Datopotamab IgG with double histidine substitution
QVQLVQSGAEVKKPGASVKVSCKASGYTFTTAGMQWVRQAPGQGLEWMGWINHHSGVPKYAEDFKGRVTISADTSTSTAYLQLSSLKSEDTAVYYCARSGFGSHYWYFDVWGQGTLVTVSS

SEQ ID NO: 400 - protein, 121 aa, synthetic construct
  REGION 1..121: note = VH domain of Datopotamab IgG with double histidine substitution
QVQLVQSGAEVKKPGASVKVSCKASGYTFTTAGMQWVRQAPGQGLEWMGWINTHSGVPKYAEDFKGRVTISADTSTSTAYLQLSSLKSEDTAVYYCAHSGHGSSYWYFDVWGQGTLVTVSS

SEQ ID NO: 401 - protein, 121 aa, synthetic construct
  REGION 1..121: note = VH domain of Datopotamab IgG with double histidine substitution
QVQLVQSGAEVKKPGASVKVSCKASGYTFTTAGMQWVRQAPGQGLEWMGWINTHSGVPKYAEDFKGRVTISADTSTSTAYLQLSSLKSEDTAVYYCAHSGFGSHYWYFDVWGQGTLVTVSS

SEQ ID NO: 402 - protein, 121 aa, synthetic construct
  REGION 1..121: note = VH domain of Datopotamab IgG with double histidine substitution
QVQLVQSGAEVKKPGASVKVSCKASGYTFTTAGMQWVRQAPGQGLEWMGWINTHSGVPKYAEDFKGRVTISADTSTSTAYLQLSSLKSEDTAVYYCARSGHGSHYWYFDVWGQGTLVTVSS

SEQ ID NO: 403 - protein, 121 aa, synthetic construct
  REGION 1..121: note = VH domain of Datopotamab IgG with triple histidine substitution
QVQLVQSGAEVKKPGASVKVSCKASGYTFTTAGMQWVRQAPGQGLEWMGWINHHSGVPKYAEDFKGRVTISADTSTSTAYLQLSSLKSEDTAVYYCAHSGHGSSYWYFDVWGQGTLVTVSS

SEQ ID NO: 404 - protein, 121 aa, synthetic construct
  REGION 1..121: note = VH domain of Datopotamab IgG with triple histidine substitution
QVQLVQSGAEVKKPGASVKVSCKASGYTFTTAGMQWVRQAPGQGLEWMGWINHHSGVPKYAEDFKGRVTISADTSTSTAYLQLSSLKSEDTAVYYCAHSGFGSHYWYFDVWGQGTLVTVSS

SEQ ID NO: 405 - protein, 121 aa, synthetic construct
  REGION 1..121: note = VH domain of Datopotamab IgG with triple histidine substitution
QVQLVQSGAEVKKPGASVKVSCKASGYTFTTAGMQWVRQAPGQGLEWMGWINHHSGVPKYAEDFKGRVTISADTSTSTAYLQLSSLKSEDTAVYYCARSGHGSHYWYFDVWGQGTLVTVSS

SEQ ID NO: 406 - protein, 121 aa, synthetic construct
  REGION 1..121: note = VH domain of Datopotamab IgG with triple histidine substitution
QVQLVQSGAEVKKPGASVKVSCKASGYTFTTAGMQWVRQAPGQGLEWMGWINTHSGVPKYAEDFKGRVTISADTSTSTAYLQLSSLKSEDTAVYYCAHSGHGSHYWYFDVWGQGTLVTVSS

SEQ ID NO: 407 - protein, 107 aa, synthetic construct
  REGION 1..107: note = VL domain of Datopotamab IgG with double histidine substitution
DIQMTQSPSSLSASVGDRVTITCKASQHVSTAVAWYQQKPGKAPKLLIYSHSYRYTGVPSRFSGSGSGTDFTLTISSLQPEDFAVYYCQQHYITPLTFGQGTKLEIK

SEQ ID NO: 408 - protein, 107 aa, synthetic construct
  REGION 1..107: note = VL domain of Datopotamab IgG with double histidine substitution
DIQMTQSPSSLSASVGDRVTITCKASQHVSTAVAWYQQKPGKAPKLLIYSAHYRYTGVPSRFSGSGSGTDFTLTISSLQPEDFAVYYCQQHYITPLTFGQGTKLEIK

SEQ ID NO: 409 - protein, 107 aa, synthetic construct
  REGION 1..107: note = VL domain of Datopotamab IgG with double histidine substitution
DIQMTQSPSSLSASVGDRVTITCKASQHVSTAVAWYQQKPGKAPKLLIYSASYRYTGVPSRFSGSGSGTDFTLTISSLQPEDFAVYYCHQHYITPLTFGQGTKLEIK

SEQ ID NO: 410 - protein, 107 aa, synthetic construct
  REGION 1..107: note = VL domain of Datopotamab IgG with double histidine substitution
DIQMTQSPSSLSASVGDRVTITCKASQHVSTAVAWYQQKPGKAPKLLIYSASYRYTGVPSRFSGSGSGTDFTLTISSLQPEDFAVYYCQQHHITPLTFGQGTKLEIK

SEQ ID NO: 411 - protein, 107 aa, synthetic construct
  REGION 1..107: note = VL domain of Datopotamab IgG with double histidine substitution
DIQMTQSPSSLSASVGDRVTITCKASQDVSTAVAWYQQKPGKAPKLLIYSHHYRYTGVPSRFSGSGSGTDFTLTISSLQPEDFAVYYCQQHYITPLTFGQGTKLEIK

SEQ ID NO: 412 - protein, 107 aa, synthetic construct
  REGION 1..107: note = VL domain of Datopotamab IgG with double histidine substitution
DIQMTQSPSSLSASVGDRVTITCKASQDVSTAVAWYQQKPGKAPKLLIYSHSYRYTGVPSRFSGSGSGTDFTLTISSLQPEDFAVYYCHQHYITPLTFGQGTKLEIK

SEQ ID NO: 413 - protein, 107 aa, synthetic construct
  REGION 1..107: note = VL domain of Datopotamab IgG with double histidine substitution
DIQMTQSPSSLSASVGDRVTITCKASQDVSTAVAWYQQKPGKAPKLLIYSHSYRYTGVPSRFSGSGSGTDFTLTISSLQPEDFAVYYCQQHHITPLTFGQGTKLEIK

SEQ ID NO: 414 - protein, 107 aa, synthetic construct
  REGION 1..107: note = VL domain of Datopotamab IgG with double histidine substitution
DIQMTQSPSSLSASVGDRVTITCKASQDVSTAVAWYQQKPGKAPKLLIYSAHYRYTGVPSRFSGSGSGTDFTLTISSLQPEDFAVYYCHQHYITPLTFGQGTKLEIK

SEQ ID NO: 415 - protein, 107 aa, synthetic construct
  REGION 1..107: note = VL domain of Datopotamab IgG with double histidine substitution
DIQMTQSPSSLSASVGDRVTITCKASQDVSTAVAWYQQKPGKAPKLLIYSAHYRYTGVPSRFSGSGSGTDFTLTISSLQPEDFAVYYCQQHHITPLTFGQGTKLEIK

SEQ ID NO: 416 - protein, 107 aa, synthetic construct
  REGION 1..107: note = VL domain of Datopotamab IgG with double histidine substitution
DIQMTQSPSSLSASVGDRVTITCKASQDVSTAVAWYQQKPGKAPKLLIYSASYRYTGVPSRFSGSGSGTDFTLTISSLQPEDFAVYYCHQHHITPLTFGQGTKLEIK

SEQ ID NO: 417 - protein, 107 aa, synthetic construct
  REGION 1..107: note = VL domain of Datopotamab IgG with triple histidine substitution
DIQMTQSPSSLSASVGDRVTITCKASQHVSTAVAWYQQKPGKAPKLLIYSHHYRYTGVPSRFSGSGSGTDFTLTISSLQPEDFAVYYCQQHYITPLTFGQGTKLEIK

SEQ ID NO: 418 - protein, 107 aa, synthetic construct
  REGION 1..107: note = VL domain of Datopotamab IgG with triple histidine substitution
DIQMTQSPSSLSASVGDRVTITCKASQHVSTAVAWYQQKPGKAPKLLIYSHSYRYTGVPSRFSGSGSGTDFTLTISSLQPEDFAVYYCHQHYITPLTFGQGTKLEIK

SEQ ID NO: 419 - protein, 107 aa, synthetic construct
  REGION 1..107: note = VL domain of Datopotamab IgG with triple histidine substitution
DIQMTQSPSSLSASVGDRVTITCKASQHVSTAVAWYQQKPGKAPKLLIYSHSYRYTGVPSRFSGSGSGTDFTLTISSLQPEDFAVYYCQQHHITPLTFGQGTKLEIK

SEQ ID NO: 420 - protein, 107 aa, synthetic construct
  REGION 1..107: note = VL domain of Datopotamab IgG with triple histidine substitution
DIQMTQSPSSLSASVGDRVTITCKASQHVSTAVAWYQQKPGKAPKLLIYSAHYRYTGVPSRFSGSGSGTDFTLTISSLQPEDFAVYYCHQHYITPLTFGQGTKLEIK

SEQ ID NO: 421 - protein, 107 aa, synthetic construct
  REGION 1..107: note = VL domain of Datopotamab IgG with triple histidine substitution
DIQMTQSPSSLSASVGDRVTITCKASQHVSTAVAWYQQKPGKAPKLLIYSAHYRYTGVPSRFSGSGSGTDFTLTISSLQPEDFAVYYCQQHHITPLTFGQGTKLEIK

SEQ ID NO: 422 - protein, 107 aa, synthetic construct
  REGION 1..107: note = VL domain of Datopotamab IgG with triple histidine substitution
DIQMTQSPSSLSASVGDRVTITCKASQHVSTAVAWYQQKPGKAPKLLIYSASYRYTGVPSRFSGSGSGTDFTLTISSLQPEDFAVYYCHQHHITPLTFGQGTKLEIK

SEQ ID NO: 423 - protein, 107 aa, synthetic construct
  REGION 1..107: note = VL domain of Datopotamab IgG with triple histidine substitution
DIQMTQSPSSLSASVGDRVTITCKASQDVSTAVAWYQQKPGKAPKLLIYSHHYRYTGVPSRFSGSGSGTDFTLTISSLQPEDFAVYYCHQHYITPLTFGQGTKLEIK

SEQ ID NO: 424 - protein, 107 aa, synthetic construct
  REGION 1..107: note = VL domain of Datopotamab IgG with triple histidine substitution
DIQMTQSPSSLSASVGDRVTITCKASQDVSTAVAWYQQKPGKAPKLLIYSHHYRYTGVPSRFSGSGSGTDFTLTISSLQPEDFAVYYCQQHHITPLTFGQGTKLEIK

SEQ ID NO: 425 - protein, 107 aa, synthetic construct
  REGION 1..107: note = VL domain of Datopotamab IgG with triple histidine substitution
DIQMTQSPSSLSASVGDRVTITCKASQDVSTAVAWYQQKPGKAPKLLIYSHSYRYTGVPSRFSGSGSGTDFTLTISSLQPEDFAVYYCHQHHITPLTFGQGTKLEIK

SEQ ID NO: 426 - protein, 107 aa, synthetic construct
  REGION 1..107: note = VL domain of Datopotamab IgG with triple histidine substitution
DIQMTQSPSSLSASVGDRVTITCKASQDVSTAVAWYQQKPGKAPKLLIYSAHYRYTGVPSRFSGSGSGTDFTLTISSLQPEDFAVYYCHQHHITPLTFGQGTKLEIK

SEQ ID NO: 427 - protein, 119 aa, synthetic construct
  REGION 1..119: note = VH domain of hu7E6SVG IgG with double histidine substitution
QVQLQESGPGLVKPSETLSLTCTVSGGSISSYHHHWIRQPPGKGLEWIGVIWTSGVTDYNSALMGRVTISVDTSKNQFSLKLSSVTAADTAVYYCARDGDYDRYTMDYWGQGTLVTVSS

SEQ ID NO: 428 - protein, 119 aa, synthetic construct
  REGION 1..119: note = VH domain of hu7E6SVG IgG with double histidine substitution
QVQLQESGPGLVKPSETLSLTCTVSGGSISSYHVHWIRQPPGKGLEWIGVHWTSGVTDYNSALMGRVTISVDTSKNQFSLKLSSVTAADTAVYYCARDGDYDRYTMDYWGQGTLVTVSS

SEQ ID NO: 429 - protein, 119 aa, synthetic construct
  REGION 1..119: note = VH domain of hu7E6SVG IgG with double histidine substitution
QVQLQESGPGLVKPSETLSLTCTVSGGSISSYHVHWIRQPPGKGLEWIGVIWTSGHTDYNSALMGRVTISVDTSKNQFSLKLSSVTAADTAVYYCARDGDYDRYTMDYWGQGTLVTVSS

SEQ ID NO: 430 - protein, 119 aa, synthetic construct
  REGION 1..119: note = VH domain of hu7E6SVG IgG with double histidine substitution
QVQLQESGPGLVKPSETLSLTCTVSGGSISSYHVHWIRQPPGKGLEWIGVIWTSGVTDYNSALMGRVTISVDTSKNQFSLKLSSVTAADTAVYYCARDGDYHRYTMDYWGQGTLVTVSS

SEQ ID NO: 431 - protein, 119 aa, synthetic construct
  REGION 1..119: note = VH domain of hu7E6SVG IgG with double histidine substitution
QVQLQESGPGLVKPSETLSLTCTVSGGSISSYHVHWIRQPPGKGLEWIGVIWTSGVTDYNSALMGRVTISVDTSKNQFSLKLSSVTAADTAVYYCARDGDYDHYTMDYWGQGTLVTVSS

SEQ ID NO: 432 - protein, 119 aa, synthetic construct
  REGION 1..119: note = VH domain of hu7E6SVG IgG with double histidine substitution
QVQLQESGPGLVKPSETLSLTCTVSGGSISSYHVHWIRQPPGKGLEWIGVIWTSGVTDYNSALMGRVTISVDTSKNQFSLKLSSVTAADTAVYYCARDGDYDRYTHDYWGQGTLVTVSS

SEQ ID NO: 433 - protein, 119 aa, synthetic construct
  REGION 1..119: note = VH domain of hu7E6SVG IgG with double histidine substitution
QVQLQESGPGLVKPSETLSLTCTVSGGSISSYGHHWIRQPPGKGLEWIGVHWTSGVTDYNSALMGRVTISVDTSKNQFSLKLSSVTAADTAVYYCARDGDYDRYTMDYWGQGTLVTVSS

SEQ ID NO: 434 - protein, 119 aa, synthetic construct
  REGION 1..119: note = VH domain of hu7E6SVG IgG with double histidine substitution
QVQLQESGPGLVKPSETLSLTCTVSGGSISSYGHHWIRQPPGKGLEWIGVIWTSGHTDYNSALMGRVTISVDTSKNQFSLKLSSVTAADTAVYYCARDGDYDRYTMDYWGQGTLVTVSS

SEQ ID NO: 435 - protein, 119 aa, synthetic construct
  REGION 1..119: note = VH domain of hu7E6SVG IgG with double histidine substitution
QVQLQESGPGLVKPSETLSLTCTVSGGSISSYGHHWIRQPPGKGLEWIGVIWTSGVTDYNSALMGRVTISVDTSKNQFSLKLSSVTAADTAVYYCARDGDYHRYTMDYWGQGTLVTVSS

SEQ ID NO: 436 - protein, 119 aa, synthetic construct
  REGION 1..119: note = VH domain of hu7E6SVG IgG with double histidine substitution
QVQLQESGPGLVKPSETLSLTCTVSGGSISSYGHHWIRQPPGKGLEWIGVIWTSGVTDYNSALMGRVTISVDTSKNQFSLKLSSVTAADTAVYYCARDGDYDHYTMDYWGQGTLVTVSS

SEQ ID NO: 437 - protein, 119 aa, synthetic construct
  REGION 1..119: note = VH domain of hu7E6SVG IgG with double histidine substitution
QVQLQESGPGLVKPSETLSLTCTVSGGSISSYGHHWIRQPPGKGLEWIGVIWTSGVTDYNSALMGRVTISVDTSKNQFSLKLSSVTAADTAVYYCARDGDYDRYTHDYWGQGTLVTVSS

SEQ ID NO: 438 - protein, 119 aa, synthetic construct
  REGION 1..119: note = VH domain of hu7E6SVG IgG with double histidine substitution
QVQLQESGPGLVKPSETLSLTCTVSGGSISSYGVHWIRQPPGKGLEWIGVHWTSGHTDYNSALMGRVTISVDTSKNQFSLKLSSVTAADTAVYYCARDGDYDRYTMDYWGQGTLVTVSS

SEQ ID NO: 439 - protein, 119 aa, synthetic construct
  REGION 1..119: note = VH domain of hu7E6SVG IgG with double histidine substitution
QVQLQESGPGLVKPSETLSLTCTVSGGSISSYGVHWIRQPPGKGLEWIGVHWTSGVTDYNSALMGRVTISVDTSKNQFSLKLSSVTAADTAVYYCARDGDYHRYTMDYWGQGTLVTVSS

SEQ ID NO: 440 - protein, 119 aa, synthetic construct
  REGION 1..119: note = VH domain of hu7E6SVG IgG with double histidine substitution
QVQLQESGPGLVKPSETLSLTCTVSGGSISSYGVHWIRQPPGKGLEWIGVHWTSGVTDYNSALMGRVTISVDTSKNQFSLKLSSVTAADTAVYYCARDGDYDHYTMDYWGQGTLVTVSS

SEQ ID NO: 441 - protein, 119 aa, synthetic construct
  REGION 1..119: note = VH domain of hu7E6SVG IgG with double histidine substitution
QVQLQESGPGLVKPSETLSLTCTVSGGSISSYGVHWIRQPPGKGLEWIGVHWTSGVTDYNSALMGRVTISVDTSKNQFSLKLSSVTAADTAVYYCARDGDYDRYTHDYWGQGTLVTVSS

SEQ ID NO: 442 - protein, 119 aa, synthetic construct
  REGION 1..119: note = VH domain of hu7E6SVG IgG with double histidine substitution
QVQLQESGPGLVKPSETLSLTCTVSGGSISSYGVHWIRQPPGKGLEWIGVIWTSGHTDYNSALMGRVTISVDTSKNQFSLKLSSVTAADTAVYYCARDGDYHRYTMDYWGQGTLVTVSS

SEQ ID NO: 443 - protein, 119 aa, synthetic construct
  REGION 1..119: note = VH domain of hu7E6SVG IgG with double histidine substitution
QVQLQESGPGLVKPSETLSLTCTVSGGSISSYGVHWIRQPPGKGLEWIGVIWTSGHTDYNSALMGRVTISVDTSKNQFSLKLSSVTAADTAVYYCARDGDYDHYTMDYWGQGTLVTVSS

SEQ ID NO: 444 - protein, 119 aa, synthetic construct
  REGION 1..119: note = VH domain of hu7E6SVG IgG with double histidine substitution
QVQLQESGPGLVKPSETLSLTCTVSGGSISSYGVHWIRQPPGKGLEWIGVIWTSGHTDYNSALMGRVTISVDTSKNQFSLKLSSVTAADTAVYYCARDGDYDRYTHDYWGQGTLVTVSS

SEQ ID NO: 445 - protein, 119 aa, synthetic construct
  REGION 1..119: note = VH domain of hu7E6SVG IgG with double histidine substitution
QVQLQESGPGLVKPSETLSLTCTVSGGSISSYGVHWIRQPPGKGLEWIGVIWTSGVTDYNSALMGRVTISVDTSKNQFSLKLSSVTAADTAVYYCARDGDYHHYTMDYWGQGTLVTVSS

SEQ ID NO: 446 - protein, 119 aa, synthetic construct
  REGION 1..119: note = VH domain of hu7E6SVG IgG with double histidine substitution
QVQLQESGPGLVKPSETLSLTCTVSGGSISSYGVHWIRQPPGKGLEWIGVIWTSGVTDYNSALMGRVTISVDTSKNQFSLKLSSVTAADTAVYYCARDGDYHRYTHDYWGQGTLVTVSS

SEQ ID NO: 447 - protein, 119 aa, synthetic construct
  REGION 1..119: note = VH domain of hu7E6SVG IgG with double histidine substitution
QVQLQESGPGLVKPSETLSLTCTVSGGSISSYGVHWIRQPPGKGLEWIGVIWTSGVTDYNSALMGRVTISVDTSKNQFSLKLSSVTAADTAVYYCARDGDYDHYTHDYWGQGTLVTVSS

SEQ ID NO: 448 - protein, 119 aa, synthetic construct
  REGION 1..119: note = VH domain of hu7E6SVG IgG with triple histidine substitution
QVQLQESGPGLVKPSETLSLTCTVSGGSISSYHVHWIRQPPGKGLEWIGVHWTSGHTDYNSALMGRVTISVDTSKNQFSLKLSSVTAADTAVYYCARDGDYDRYTMDYWGQGTLVTVSS

SEQ ID NO: 449 - protein, 119 aa, synthetic construct
  REGION 1..119: note = VH domain of hu7E6SVG IgG with triple histidine substitution
QVQLQESGPGLVKPSETLSLTCTVSGGSISSYHVHWIRQPPGKGLEWIGVHWTSGVTDYNSALMGRVTISVDTSKNQFSLKLSSVTAADTAVYYCARDGDYDHYTMDYWGQGTLVTVSS

SEQ ID NO: 450 - protein, 119 aa, synthetic construct
  REGION 1..119: note = VH domain of hu7E6SVG IgG with triple histidine substitution
QVQLQESGPGLVKPSETLSLTCTVSGGSISSYHVHWIRQPPGKGLEWIGVHWTSGVTDYNSALMGRVTISVDTSKNQFSLKLSSVTAADTAVYYCARDGDYDRYTHDYWGQGTLVTVSS

SEQ ID NO: 451 - protein, 119 aa, synthetic construct
  REGION 1..119: note = VH domain of hu7E6SVG IgG with triple histidine substitution
QVQLQESGPGLVKPSETLSLTCTVSGGSISSYHVHWIRQPPGKGLEWIGVIWTSGHTDYNSALMGRVTISVDTSKNQFSLKLSSVTAADTAVYYCARDGDYDHYTMDYWGQGTLVTVSS

SEQ ID NO: 452 - protein, 119 aa, synthetic construct
  REGION 1..119: note = VH domain of hu7E6SVG IgG with triple histidine substitution
QVQLQESGPGLVKPSETLSLTCTVSGGSISSYHVHWIRQPPGKGLEWIGVIWTSGHTDYNSALMGRVTISVDTSKNQFSLKLSSVTAADTAVYYCARDGDYDRYTHDYWGQGTLVTVSS

SEQ ID NO: 453 - protein, 119 aa, synthetic construct
  REGION 1..119: note = VH domain of hu7E6SVG IgG with triple histidine substitution
QVQLQESGPGLVKPSETLSLTCTVSGGSISSYHVHWIRQPPGKGLEWIGVIWTSGVTDYNSALMGRVTISVDTSKNQFSLKLSSVTAADTAVYYCARDGDYDHYTHDYWGQGTLVTVSS

SEQ ID NO: 454 - protein, 119 aa, synthetic construct
  REGION 1..119: note = VH domain of hu7E6SVG IgG with triple histidine substitution
QVQLQESGPGLVKPSETLSLTCTVSGGSISSYGVHWIRQPPGKGLEWIGVHWTSGHTDYNSALMGRVTISVDTSKNQFSLKLSSVTAADTAVYYCARDGDYDHYTMDYWGQGTLVTVSS

SEQ ID NO: 455 - protein, 119 aa, synthetic construct
  REGION 1..119: note = VH domain of hu7E6SVG IgG with triple histidine substitution
QVQLQESGPGLVKPSETLSLTCTVSGGSISSYGVHWIRQPPGKGLEWIGVHWTSGHTDYNSALMGRVTISVDTSKNQFSLKLSSVTAADTAVYYCARDGDYDRYTHDYWGQGTLVTVSS

SEQ ID NO: 456 - protein, 119 aa, synthetic construct
  REGION 1..119: note = VH domain of hu7E6SVG IgG with triple histidine substitution
QVQLQESGPGLVKPSETLSLTCTVSGGSISSYGVHWIRQPPGKGLEWIGVHWTSGVTDYNSALMGRVTISVDTSKNQFSLKLSSVTAADTAVYYCARDGDYDHYTHDYWGQGTLVTVSS

SEQ ID NO: 457 - protein, 119 aa, synthetic construct
  REGION 1..119: note = VH domain of hu7E6SVG IgG with triple histidine substitution
QVQLQESGPGLVKPSETLSLTCTVSGGSISSYGVHWIRQPPGKGLEWIGVIWTSGHTDYNSALMGRVTISVDTSKNQFSLKLSSVTAADTAVYYCARDGDYDHYTHDYWGQGTLVTVSS

SEQ ID NO: 458 - protein, 111 aa, synthetic construct
  REGION 1..111: note = VL domain of hu7E6SVG IgG with double histidine substitution
DIVMTQSPDSLAVSLGERATINCRASKSVSTSGYSYMHWYQQKPGQPPKLLIYLASNLHHGVPDRFSGSGSGTDFTLTISSLQAEDVAVYYCQHSRELPYTFGQGTKLEIK

SEQ ID NO: 459 - protein, 111 aa, synthetic construct
  REGION 1..111: note = VL domain of hu7E6SVG IgG with double histidine substitution
DIVMTQSPDSLAVSLGERATINCRASKSVSTSGYSYMHWYQQKPGQPPKLLIYLASNLHSGVPDRFSGSGSGTDFTLTISSLQAEDVAVYYCHHSRELPYTFGQGTKLEIK

SEQ ID NO: 460 - protein, 111 aa, synthetic construct
  REGION 1..111: note = VL domain of hu7E6SVG IgG with double histidine substitution
DIVMTQSPDSLAVSLGERATINCRASKSVSTSGYSYMHWYQQKPGQPPKLLIYLASNLHSGVPDRFSGSGSGTDFTLTISSLQAEDVAVYYCQASRELPYTFGQGTKLEIK

SEQ ID NO: 461 - protein, 111 aa, synthetic construct
  REGION 1..111: note = VL domain of hu7E6SVG IgG with double histidine substitution
DIVMTQSPDSLAVSLGERATINCRASKSVSTSGYSYMHWYQQKPGQPPKLLIYLASNLHSGVPDRFSGSGSGTDFTLTISSLQAEDVAVYYCQHSRHLPYTFGQGTKLEIK

SEQ ID NO: 462 - protein, 111 aa, synthetic construct
  REGION 1..111: note = VL domain of hu7E6SVG IgG with double histidine substitution
DIVMTQSPDSLAVSLGERATINCRASKSVSTSGYSYMHWYQQKPGQPPKLLIYLASNLHSGVPDRFSGSGSGTDFTLTISSLQAEDVAVYYCQHSRELPYHFGQGTKLEIK

SEQ ID NO: 463 - protein, 111 aa, synthetic construct
  REGION 1..111: note = VL domain of hu7E6SVG IgG with double histidine substitution
DIVMTQSPDSLAVSLGERATINCRASKSVSTSGYSYMHWYQQKPGQPPKLLIYLASNLEHGVPDRFSGSGSGTDFTLTISSLQAEDVAVYYCHHSRELPYTFGQGTKLEIK

SEQ ID NO: 464 - protein, 111 aa, synthetic construct
  REGION 1..111: note = VL domain of hu7E6SVG IgG with double histidine substitution
DIVMTQSPDSLAVSLGERATINCRASKSVSTSGYSYMHWYQQKPGQPPKLLIYLASNLEHGVPDRFSGSGSGTDFTLTISSLQAEDVAVYYCQASRELPYTFGQGTKLEIK

SEQ ID NO: 465 - protein, 111 aa, synthetic construct
  REGION 1..111: note = VL domain of hu7E6SVG IgG with double histidine substitution
DIVMTQSPDSLAVSLGERATINCRASKSVSTSGYSYMHWYQQKPGQPPKLLIYLASNLEHGVPDRFSGSGSGTDFTLTISSLQAEDVAVYYCQHSRHLPYTFGQGTKLEIK

SEQ ID NO: 466 - protein, 111 aa, synthetic construct
  REGION 1..111: note = VL domain of hu7E6SVG IgG with double histidine substitution
DIVMTQSPDSLAVSLGERATINCRASKSVSTSGYSYMHWYQQKPGQPPKLLIYLASNLEHGVPDRFSGSGSGTDFTLTISSLQAEDVAVYYCQHSRELPYHFGQGTKLEIK

SEQ ID NO: 467 - protein, 111 aa, synthetic construct
  REGION 1..111: note = VL domain of hu7E6SVG IgG with double histidine substitution
DIVMTQSPDSLAVSLGERATINCRASKSVSTSGYSYMHWYQQKPGQPPKLLIYLASNLESGVPDRFSGSGSGTDFTLTISSLQAEDVAVYYCHASRELPYTFGQGTKLEIK

SEQ ID NO: 468 - protein, 111 aa, synthetic construct
  REGION 1..111: note = VL domain of hu7E6SVG IgG with double histidine substitution
DIVMTQSPDSLAVSLGERATINCRASKSVSTSGYSYMHWYQQKPGQPPKLLIYLASNLESGVPDRFSGSGSGTDFTLTISSLQAEDVAVYYCHHSRHLPYTFGQGTKLEIK

SEQ ID NO: 469 - protein, 111 aa, synthetic construct
  REGION 1..111: note = VL domain of hu7E6SVG IgG with double histidine substitution
DIVMTQSPDSLAVSLGERATINCRASKSVSTSGYSYMHWYQQKPGQPPKLLIYLASNLESGVPDRFSGSGSGTDFTLTISSLQAEDVAVYYCHHSRELPYHFGQGTKLEIK

SEQ ID NO: 470 - protein, 111 aa, synthetic construct
  REGION 1..111: note = VL domain of hu7E6SVG IgG with double histidine substitution
DIVMTQSPDSLAVSLGERATINCRASKSVSTSGYSYMHWYQQKPGQPPKLLIYLASNLESGVPDRFSGSGSGTDFTLTISSLQAEDVAVYYCQASRHLPYTFGQGTKLEIK

SEQ ID NO: 471 - protein, 111 aa, synthetic construct
  REGION 1..111: note = VL domain of hu7E6SVG IgG with double histidine substitution
DIVMTQSPDSLAVSLGERATINCRASKSVSTSGYSYMHWYQQKPGQPPKLLIYLASNLESGVPDRFSGSGSGTDFTLTISSLQAEDVAVYYCQASRELPYHFGQGTKLEIK

SEQ ID NO: 472 - protein, 111 aa, synthetic construct
  REGION 1..111: note = VL domain of hu7E6SVG IgG with double histidine substitution
DIVMTQSPDSLAVSLGERATINCRASKSVSTSGYSYMHWYQQKPGQPPKLLIYLASNLESGVPDRFSGSGSGTDFTLTISSLQAEDVAVYYCQHSRHLPYHFGQGTKLEIK

SEQ ID NO: 473 - protein, 111 aa, synthetic construct
  REGION 1..111: note = VL domain of hu7E6SVG IgG with triple histidine substitution
DIVMTQSPDSLAVSLGERATINCRASKSVSTSGYSYMHWYQQKPGQPPKLLIYLASNLHHGVPDRFSGSGSGTDFTLTISSLQAEDVAVYYCHHSRELPYTFGQGTKLEIK

SEQ ID NO: 474 - protein, 111 aa, synthetic construct
  REGION 1..111: note = VL domain of hu7E6SVG IgG with triple histidine substitution
DIVMTQSPDSLAVSLGERATINCRASKSVSTSGYSYMHWYQQKPGQPPKLLIYLASNLHHGVPDRFSGSGSGTDFTLTISSLQAEDVAVYYCQASRELPYTFGQGTKLEIK

SEQ ID NO: 475 - protein, 111 aa, synthetic construct
  REGION 1..111: note = VL domain of hu7E6SVG IgG with triple histidine substitution
DIVMTQSPDSLAVSLGERATINCRASKSVSTSGYSYMHWYQQKPGQPPKLLIYLASNLHHGVPDRFSGSGSGTDFTLTISSLQAEDVAVYYCQHSRHLPYTFGQGTKLEIK

SEQ ID NO: 476 - protein, 111 aa, synthetic construct
  REGION 1..111: note = VL domain of hu7E6SVG IgG with triple histidine substitution
DIVMTQSPDSLAVSLGERATINCRASKSVSTSGYSYMHWYQQKPGQPPKLLIYLASNLHHGVPDRFSGSGSGTDFTLTISSLQAEDVAVYYCQHSRELPYHFGQGTKLEIK

SEQ ID NO: 477 - protein, 111 aa, synthetic construct
  REGION 1..111: note = VL domain of hu7E6SVG IgG with triple histidine substitution
DIVMTQSPDSLAVSLGERATINCRASKSVSTSGYSYMHWYQQKPGQPPKLLIYLASNLHSGVPDRFSGSGSGTDFTLTISSLQAEDVAVYYCHASRELPYTFGQGTKLEIK

SEQ ID NO: 478 - protein, 111 aa, synthetic construct
  REGION 1..111: note = VL domain of hu7E6SVG IgG with triple histidine substitution
DIVMTQSPDSLAVSLGERATINCRASKSVSTSGYSYMHWYQQKPGQPPKLLIYLASNLHSGVPDRFSGSGSGTDFTLTISSLQAEDVAVYYCHHSRHLPYTFGQGTKLEIK

SEQ ID NO: 479 - protein, 111 aa, synthetic construct
  REGION 1..111: note = VL domain of hu7E6SVG IgG with triple histidine substitution
DIVMTQSPDSLAVSLGERATINCRASKSVSTSGYSYMHWYQQKPGQPPKLLIYLASNLHSGVPDRFSGSGSGTDFTLTISSLQAEDVAVYYCHHSRELPYHFGQGTKLEIK

SEQ ID NO: 480 - protein, 111 aa, synthetic construct
  REGION 1..111: note = VL domain of hu7E6SVG IgG with triple histidine substitution
DIVMTQSPDSLAVSLGERATINCRASKSVSTSGYSYMHWYQQKPGQPPKLLIYLASNLHSGVPDRFSGSGSGTDFTLTISSLQAEDVAVYYCQASRHLPYTFGQGTKLEIK

SEQ ID NO: 481 - protein, 111 aa, synthetic construct
  REGION 1..111: note = VL domain of hu7E6SVG IgG with triple histidine substitution
DIVMTQSPDSLAVSLGERATINCRASKSVSTSGYSYMHWYQQKPGQPPKLLIYLASNLHSGVPDRFSGSGSGTDFTLTISSLQAEDVAVYYCQASRELPYHFGQGTKLEIK

SEQ ID NO: 482 - protein, 111 aa, synthetic construct
  REGION 1..111: note = VL domain of hu7E6SVG IgG with triple histidine substitution
DIVMTQSPDSLAVSLGERATINCRASKSVSTSGYSYMHWYQQKPGQPPKLLIYLASNLHSGVPDRFSGSGSGTDFTLTISSLQAEDVAVYYCQHSRHLPYHFGQGTKLEIK

SEQ ID NO: 483 - protein, 111 aa, synthetic construct
  REGION 1..111: note = VL domain of hu7E6SVG IgG with triple histidine substitution
DIVMTQSPDSLAVSLGERATINCRASKSVSTSGYSYMHWYQQKPGQPPKLLIYLASNLEHGVPDRFSGSGSGTDFTLTISSLQAEDVAVYYCHASRELPYTFGQGTKLEIK

SEQ ID NO: 484 - protein, 111 aa, synthetic construct
  REGION 1..111: note = VL domain of hu7E6SVG IgG with triple histidine substitution
DIVMTQSPDSLAVSLGERATINCRASKSVSTSGYSYMHWYQQKPGQPPKLLIYLASNLEHGVPDRFSGSGSGTDFTLTISSLQAEDVAVYYCHHSRHLPYTFGQGTKLEIK

SEQ ID NO: 485 - protein, 111 aa, synthetic construct
  REGION 1..111: note = VL domain of hu7E6SVG IgG with triple histidine substitution
DIVMTQSPDSLAVSLGERATINCRASKSVSTSGYSYMHWYQQKPGQPPKLLIYLASNLEHGVPDRFSGSGSGTDFTLTISSLQAEDVAVYYCHHSRELPYHFGQGTKLEIK

SEQ ID NO: 486 - protein, 111 aa, synthetic construct
  REGION 1..111: note = VL domain of hu7E6SVG IgG with triple histidine substitution
DIVMTQSPDSLAVSLGERATINCRASKSVSTSGYSYMHWYQQKPGQPPKLLIYLASNLEHGVPDRFSGSGSGTDFTLTISSLQAEDVAVYYCQASRHLPYTFGQGTKLEIK

SEQ ID NO: 487 - protein, 111 aa, synthetic construct
  REGION 1..111: note = VL domain of hu7E6SVG IgG with triple histidine substitution
DIVMTQSPDSLAVSLGERATINCRASKSVSTSGYSYMHWYQQKPGQPPKLLIYLASNLEHGVPDRFSGSGSGTDFTLTISSLQAEDVAVYYCQASRELPYHFGQGTKLEIK

SEQ ID NO: 488 - protein, 111 aa, synthetic construct
  REGION 1..111: note = VL domain of hu7E6SVG IgG with triple histidine substitution
DIVMTQSPDSLAVSLGERATINCRASKSVSTSGYSYMHWYQQKPGQPPKLLIYLASNLEHGVPDRFSGSGSGTDFTLTISSLQAEDVAVYYCQHSRHLPYHFGQGTKLEIK

SEQ ID NO: 489 - protein, 111 aa, synthetic construct
  REGION 1..111: note = VL domain of hu7E6SVG IgG with triple histidine substitution
DIVMTQSPDSLAVSLGERATINCRASKSVSTSGYSYMHWYQQKPGQPPKLLIYLASNLESGVPDRFSGSGSGTDFTLTISSLQAEDVAVYYCHASRHLPYTFGQGTKLEIK

SEQ ID NO: 490 - protein, 111 aa, synthetic construct
  REGION 1..111: note = VL domain of hu7E6SVG IgG with triple histidine substitution
DIVMTQSPDSLAVSLGERATINCRASKSVSTSGYSYMHWYQQKPGQPPKLLIYLASNLESGVPDRFSGSGSGTDFTLTISSLQAEDVAVYYCHASRELPYHFGQGTKLEIK

SEQ ID NO: 491 - protein, 111 aa, synthetic construct
  REGION 1..111: note = VL domain of hu7E6SVG IgG with triple histidine substitution
DIVMTQSPDSLAVSLGERATINCRASKSVSTSGYSYMHWYQQKPGQPPKLLIYLASNLESGVPDRFSGSGSGTDFTLTISSLQAEDVAVYYCHHSRHLPYHFGQGTKLEIK

SEQ ID NO: 492 - protein, 111 aa, synthetic construct
  REGION 1..111: note = VL domain of hu7E6SVG IgG with triple histidine substitution
DIVMTQSPDSLAVSLGERATINCRASKSVSTSGYSYMHWYQQKPGQPPKLLIYLASNLESGVPDRFSGSGSGTDFTLTISSLQAEDVAVYYCQASRHLPYHFGQGTKLEIK

(19) United States
(12) Patent Application Publication
(10) Pub. No.: US 2026/0274985 A1
(43) Pub. Date: Sep. 17, 2026

(54) TROP2-BINDING PROTEINS AND USES THEREOF

(71) Applicant: MYTHIC THERAPEUTICS, INC., Waltham, MA (US)

(72) Inventors: Brian Fiske, Waltham, MA (US); Nimish Gera, Waltham, MA (US); Thomas Chittenden, Sudbury, MA (US); David Niquille, Boston, MA (US)

(73) Assignee: MYTHIC THERAPEUTICS, INC., Waltham, MA (US)

(21) Appl. No.: 19/469,445

(22) PCT Filed: Mar. 29, 2024
(86) PCT No.: PCT/US2024/022221

§ 371 Date: Sep. 26, 2025

Related U.S. Application Data
(60) Provisional application No. 63/455,278, filed on Mar. 29, 2023.
(60) Provisional application No. 63/596,306, filed on Nov. 5, 2023.

(51) Int. Cl. C07K 16/30; A61P 35/00

(52) U.S. Cl. CPC C07K 16/30; A61P 35/00; C07K 2317/92

(57) ABSTRACT

Provided herein are antigen-binding protein constructs (ABPCs) that bind TROP2 and uses of the same.

### CROSS-REFERENCE TO RELATED APPLICATIONS

This application claims priority to U.S. Provisional Patent Applications Ser. No. 63/596,306, filed Nov. 5, 2023, and 63/455,278, filed on Mar. 29, 2023, the entire contents of which are hereby incorporated by reference in their entireties.

### SEQUENCE LISTING

This application contains a Sequence Listing that has been submitted electronically as an XML file named “45395-0063WO_SL_ST26”. The XML file, created on Nov. 4, 2023, is 683,397 bytes in size, and the material contained within it is hereby incorporated by reference in its entirety.

### TECHNICAL FIELD

The present disclosure relates to the field of antigen-binding molecules.

### SUMMARY

The present invention is based on the concept that antigen-binding protein (ABP) constructs can be generated that display enhanced efficacy in toxin liberation in a target mammalian cell, an increase in endolysosomal delivery, and an increase in cell killing.

Trophoblast cell surface antigen 2 (TROP2), also known as tumor-associated calcium signal transducer 2 (TACSTD2), is a cellular target of increasing interest in the field of oncology. TROP2 is overexpressed in a number of large cancers (e.g., NSCLC, breast), and the anti-TROP2 ADC, sacituzumab govitecan (Trodelvy®), has been approved in metastatic TNBC. Another anti-TROP2 ADC, datopotamab deruxtecan, has been showing encouraging efficacy in the HR+/HER2-cohort and the TNBC cohorts in Phase 3 trials. Notably, Trodelvy has shown less efficacy in TROP2 low versus TROP2 high patients, indicating an unmet need for patients having low TROP2 expressing tumors. Moreover, TROP2 has significant expression in normal tissue, causing target mediated clearance of anti-TROP2 ADCs, and creating a challenge to selectively increase the delivery to tumor but not normal cells.

Reasoning that pH engineering of TROP2 antibodies could potentially reduce uptake of corresponding ADCs in normal tissues, Applicants sought to develop the anti-TROP2 binding proteins as disclosed herein. Such anti-TROP2 pH-ADCs hold the potential to mitigate the “on target” toxicity and widen therapeutic window as compared to existing, non-pH-dependent anti-TROP2 ADCs. The pH-ADCs of the present disclosure could also improve efficacy by increasing tumor uptake and improving the PK profile, which could be particularly beneficial for patients having tumors characterized by low TROP2 expression.

As disclosed herein, Applicant designed, produced, and extensively evaluated about 700 antigen binding protein construct (ABPC) candidates to produce scores of ABPCs exhibiting superior pH-dependence, internalization, and cytotoxicity (as ADCs) when compared to their corresponding, non-augmented control ABPCs (or control ADCs). Of these initial candidates, nearly ten percent exhibited greater than 1.4× cellular internalization over their respective control ABPCs, and many of these superior internalizers exhibited a correlative superior cytotoxicity against TROP2-positive cells. Taken together, the results disclosed herein demonstrate that cellular TROP2 can be effectively targeted using pH-engineered anti-TROP2 antibodies (and conjugates produced therefrom) exhibiting strong binding at physiologic pH but not at the acidic pH found in the endolysosomal/lysosomal compartments. Ongoing and future studies will evaluate the capabilities of these augmented anti-TROP2 ABPCs, particularly as regards their ability to carry any desired cargo into TROP2-positive cells, both in vitro and in vivo.

Accordingly, provided herein are antigen-binding protein construct (ABPCs) and pharmaceutical compositions including an effective amount of an ABPC including: a first antigen-binding domain (ABD) that is capable of specifically binding TROP2 or an epitope of TROP2 presented on the surface of a target mammalian cell, where: (a) the dissociation rate of the first ABD at a pH of about 4.0 to about 6.5 is faster than the dissociation rate at a pH of about 7.0 to about 8.0; and/or (b) the dissociation constant (KD) of the first ABD at a pH of about 4.0 to about 6.5 is greater than the KD at a pH of about 7.0 to about 8.0.

In some embodiments, the first ABD includes a heavy chain variable domain (HCVD) of an HCVD of sacituzumab, datopotamab, hu7E6SVG, or A1,2X4, each HCVD with at least one (1) histidine substitution (or alanine where a histidine was already present).

As used herein, when a histidine or an alanine substitution is recited, it may be present in one or more of the heavy chain CDRs and/or light chain CDRs, wherein each such CDR is determined by the Kabat method, IMGT, or the additive “Kabat-IMGT” approach described in Example 1 (i.e., defining a given CDR by including the amino acid residues present in either or both the Kabat and IMGT CDR definitions).

In some embodiments, the first ABD includes a light chain variable domain (LCVD) of an LCVD of sacituzumab, datopotamab, hu7E6SVG, or A1,2X4, each LCVD with at least one (1) histidine substitution (or an alanine where a histidine was already present).

In some embodiments, the first ABD includes one of (a) to (d): (a) an HCVD of sacituzumab and/or an LCVD of sacituzumab; (b) an HCVD of datopotamab and/or an LCVD of datopotamab; (c) an HCVD of hu7E6SVG and/or an LCVD of hu7E6SVG; and (d) an HCVD of A1,2X4 and/or an LCVD of A1,2X4, each HCVD or LCVD with one or more amino acids substituted with a histidine (or an alanine where a histidine was already present).

In some embodiments, the HCVD includes one of (a) to (d): (a) an HCVD of sacituzumab including SEQ ID NO: 4; (b) an HCVD of datopotamab including SEQ ID NO: 80. (c) an HCVD of hu7E6SVG including SEQ ID NO: 156; and (d) an HCVD of A1,2X4 including SEQ ID NO: 234, each HCVD with one or more amino acids substituted with a histidine (or an alanine where a histidine was already present).

In some embodiments, the LCVD includes one of (a) to (d): (a) an LCVD of sacituzumab including SEQ ID NO: 5; (b) an LCVD of datopotamab including SEQ ID NO: 81; (c) an LCVD of hu7E6SVG including SEQ ID NO: 157; and (d) an LCVD of A1,2X4 including SEQ ID NO: 235, each LCVD with one or more amino acids substituted with a histidine (or an alanine where a histidine was already present).

In some embodiments, the first TROP2 binding domain includes an HCVD of one of (a) to (d): (a) an HCVD including a CDR1, a CDR2, and a CDR3 of SEQ ID NOs: 6, 7, and 8, respectively, with collectively a total of one or more amino acid positions in SEQ ID NOs: 6, 7, and 8 substituted with a histidine; (b) an HCVD including a CDR1, a CDR2, and a CDR3 of SEQ ID NOs: 82, 83, and 84, respectively, with collectively a total of one or more amino acid positions in SEQ ID NOs: 82, 83, and 84 substituted with a histidine; (c) an HCVD including a CDR1, a CDR2, and a CDR3 of SEQ ID NOs: 158, 159, and 160, respectively, with collectively a total of one or more amino acid positions in SEQ ID NOs: 158, 159, and 160 substituted with a histidine; and (d) an HCVD including a CDR1, a CDR2, and a CDR3 of SEQ ID NOs: 236, 237, and 238, respectively, with collectively a total of one or more amino acid positions in SEQ ID NOs: 236, 237, and 238 substituted with a histidine. In each of the foregoing embodiments, an alanine may be substituted where a histidine was already present.

In some embodiments, the first ABD includes one of (a) to (d): (a) an LCVD including a CDR1, a CDR2, and a CDR3 of SEQ ID NOs: 9, 10, and 11, respectively, with collectively a total of one or more amino acid positions in SEQ ID NOs: 9, 10, and 11 substituted with a histidine; (b) an LCVD including a CDR1, a CDR2, and a CDR3 of SEQ ID NOs: 85, 86, and 87, respectively, with collectively a total of one or more amino acid positions in SEQ ID NOs: 85, 86, and 87 substituted with a histidine; (c) an LCVD including a CDR1, a CDR2, and a CDR3 of SEQ ID NOs: 161, 162, and 163, respectively, with collectively a total of one or more amino acid positions in SEQ ID NOs: 161, 162, and 163 substituted with a histidine; and (d) an LCVD including a CDR1, a CDR2, and a CDR3 of SEQ ID NOs: 239, 240, and 241, respectively, with collectively a total of one or more amino acid positions in SEQ ID NOs: 239, 240, and 241 substituted with a histidine. In each of the foregoing embodiments, an alanine may be substituted where a histidine was already present.

In some embodiments, the first ABD includes one of (a) to (d): (a) an HCVD including a CDR1, a CDR2, and a CDR3 of SEQ ID NOs: 6, 7, and 8, respectively, with collectively a total of one or more amino acid positions in SEQ ID NOs: 6, 7, and 8 substituted with a histidine; and/or an LCVD including a CDR1, a CDR2, and a CDR3 of SEQ ID NOs: 9, 10, and 11, respectively, with collectively a total of one or more amino acid positions in SEQ ID NOs: 9, 10, and 11 substituted with a histidine; (b) an HCVD including a CDR1, a CDR2, and a CDR3 of SEQ ID NOs: 82, 83, and 84, respectively, with collectively a total of one or more amino acid positions in SEQ ID NOs: 82, 83, and 84 substituted with a histidine; and/or an LCVD including a CDR1, a CDR2, and a CDR3 of SEQ ID NOs: 85, 86, and 87, respectively, with collectively a total of one or more amino acid positions in SEQ ID NOs: 85, 86, and 87 substituted with a histidine; (c) an HCVD including a CDR1, a CDR2, and a CDR3 of SEQ ID NOs: 158, 159, and 160, respectively, with collectively a total of one or more amino acid positions in SEQ ID NOs: 158, 159, and 160 substituted with a histidine; and/or an LCVD including a CDR1, a CDR2, and a CDR3 of SEQ ID NOs: 161, 162, and 163, respectively, with collectively a total of one or more amino acid positions in SEQ ID NOs: 161, 162, and 163 substituted with a histidine; and (d) an HCVD including a CDR1, a CDR2, and a CDR3 of SEQ ID NOs: 236, 237, and 238, respectively, with collectively a total of one or more amino acid positions in SEQ ID NOs: 236, 237, and 238 substituted with a histidine; and/or an LCVD including a CDR1, a CDR2, and a CDR3 of SEQ ID NOs: 239, 240, and 241, respectively, with collectively a total of one or more amino acid positions in SEQ ID NOs: 239, 240, and 241 substituted with a histidine. In each of the foregoing embodiments, an alanine may be substituted where a histidine was already present.

In some embodiments, the first ABD includes an HCVD that is one of (a) to (d): (a) an HCVD that is at least 90%, 95%, or 98% identical to SEQ ID NO: 4, where the HCVD includes a histidine at one or more positions in SEQ ID NO: 4 selected from: 29, 32, 33, 50, 52, 53, 60, 61, 62, 63, 64, 65, 66, 99, 101, 104, 105, and 108; (b) an HCVD that is at least 90%, 95%, or 98% identical to SEQ ID NO: 80, where the HCVD includes a histidine at one or more positions in SEQ ID NO: 80 selected from: 29, 53, 55, 64, 65, 98, 101, 104, 105, 107, and 108; (c) an HCVD that is at least 90%, 95%, or 98% identical to SEQ ID NO: 156, where the HCVD includes a histidine at one or more positions in SEQ ID NO: 156 selected from: 26, 29, 30, 33, 34, 51, 54, 56, 57, 59, 102, 103, and 106; and (d) an HCVD that is at least 90%, 95%, or 98% identical to SEQ ID NO: 234, where the HCVD includes a histidine at one or more positions in SEQ ID NO: 234. In each of the foregoing embodiments, an alanine may be substituted where a histidine was already present.

In some embodiments, the first ABD includes one of (a) to (d): (a) an LCVD that is at least 90%, 95%, or 98% identical to SEQ ID NO: 5, where the LCVD includes a histidine at one or more positions in SEQ ID NO: 5 selected from: 28, 29, 30, 31, 32, 33, 50, 51, 52, 53, 89, 92, 93, 94, 95, 96, and 97; (b) an LCVD that is at least 90%, 95%, or 98% identical to SEQ ID NO: 81, where the LCVD includes a histidine at one or more positions in SEQ ID NO: 81 selected from: 26, 28, 29, 30, 31, 33, 34, 50, 51, 52, 53, 54, 89, 93, 94, and 95; (c) an LCVD that is at least 90%, 95%, or 98% identical to SEQ ID NO: 157, where the LCVD includes a histidine at one or more positions in SEQ ID NO: 157 selected from 25, 32, 34, 36, 54, 56, 57, 58, 59, 60, 93, 96, 97, 99, 100, and 101; and (d) an LCVD that is at least 90%, 95%, or 98% identical to SEQ ID NO: 235, where the LCVD includes a histidine at one or more positions in SEQ ID NO: 235. In each of the foregoing embodiments, an alanine may be substituted where a histidine was already present.

In some embodiments, the first ABD includes an HCVD of one of (a) to (d): (a) an HCVD that is at least 90%, 95%, or 98% identical to SEQ ID NO: 4, where the HCVD includes a histidine at two or more positions in SEQ ID NO: 4 selected from: 29, 32, 33, 50, 52, 53, 60, 61, 62, 63, 64, 65, 66, 99, 101, 104, 105, and 108; (b) an HCVD that is at least 90%, 95%, or 98% identical to SEQ ID NO: 80, where the HCVD includes a histidine at two or more positions in SEQ ID NO: 80 selected from: 29, 53, 55, 64, 65, 98, 101, 104, 105, 107, and 108; (c) an HCVD that is at least 90%, 95%, or 98% identical to SEQ ID NO: 156, where the HCVD includes a histidine at two or more positions in SEQ ID NO: 156 selected from: 26, 29, 30, 33, 34, 51, 54, 56, 57, 59, 102, 103, and 106; and (d) an HCVD that is at least 90%, 95%, or 98% identical to SEQ ID NO: 234, where the HCVD includes a histidine at two or more positions in SEQ ID NO: 234. In each of the foregoing embodiments, an alanine may be substituted where a histidine was already present.

In some embodiments, the first ABD includes an LCVD of one of (a) to (d): (a) an LCVD that is at least 90%, 95%, or 98% identical to SEQ ID NO: 5, where the LCVD includes a histidine at two or more positions in SEQ ID NO: 5 selected from: 28, 29, 30, 31, 32, 33, 50, 51, 52, 53, 89, 92, 93, 94, 95, 96, and 97; (b) an LCVD that is at least 90%, 95%, or 98% identical to SEQ ID NO: 81, wherein the LCVD includes a histidine at two or more positions in SEQ ID NO: 81 selected from: 26, 28, 29, 30, 31, 33, 34, 50, 51, 52, 53, 54, 89, 93, 94, and 95; (c) an LCVD that is at least 90%, 95%, or 98% identical to SEQ ID NO: 157, wherein the LCVD includes a histidine at two or more positions in SEQ ID NO: 157 selected from: 25, 32, 34, 36, 54, 56, 57, 58, 59, 60, 93, 96, 97, 99, 100, and 101; and (d) an LCVD that is at least 90%, 95%, or 98% identical to SEQ ID NO: 235, wherein the LCVD includes a histidine at two or more positions in SEQ ID NO: 235. In each of the foregoing embodiments, an alanine may be substituted where a histidine was already present.

In some embodiments, the first ABD includes one of (a) to (d): (a) an HCVD that is at least 90%, 95%, or 98% identical to SEQ ID NO: 4, where the HCVD includes a histidine at one or more positions in SEQ ID NO: 4 selected from: 29, 32, 33, 50, 52, 53, 60, 61, 62, 63, 64, 65, 66, 99, 101, 104, 105, and 108; and/or an LCVD that is at least 90%, 95%, or 98% identical to SEQ ID NO: 5, where the LCVD includes a histidine at one or more positions in SEQ ID NO: 5 selected from: 28, 29, 30, 31, 32, 33, 50, 51, 52, 53, 89, 92, 93, 94, 95, 96, and 97; (b) an HCVD that is at least 90%, 95%, or 98% identical to SEQ ID NO: 80, where the HCVD includes a histidine at one or more positions in SEQ ID NO: 80 selected from: 29, 53, 55, 64, 65, 98, 101, 104, 105, 107, and 108; and/or an LCVD that is at least 90%, 95%, or 98% identical to SEQ ID NO: 81, where the LCVD includes a histidine at one or more positions in SEQ ID NO: 81 selected from: 26, 28, 29, 30, 31, 33, 34, 50, 51, 52, 53, 54, 89, 93, 94, and 95; (c) an HCVD that is at least 90%, 95%, or 98% identical to SEQ LD NO: 156, wherein the HCVD includes a histidine at one or more positions in SEQ ID NO: 156 selected from: 26, 29, 30, 33, 34, 51, 54, 56, 57, 59, 102, 103, and 106; and/or an LCVD that is at least 90%, 95%, or 98% identical to SEQ ID NO: 157, where the LCVD includes a histidine at one or more positions in SEQ ID NO: 157 selected from 25, 32, 34, 36, 54, 56, 57, 58, 59, 60, 93, 96, 97, 99, 100, and 101; and (d) an HCVD that is at least 90%, 95%, or 98% identical to SEQ ID NO: 234, wherein the HCVD includes a histidine at one or more positions in SEQ ID NO: 234; and/or an LCVD that is at least 90%, 95%, or 98% identical to SEQ ID NO: 235, where the LCVD includes a histidine at one or more positions in SEQ ID NO: 235. In each of the foregoing embodiments, an alanine may be substituted where a histidine was already present.

In some embodiments, the first ABD includes one of (a) to (d): (a) an HCVD of SEQ ID NO: 4, one of the sequences set forth in SEQ ID NOs: 12-52, or one of the sequences set forth in SEQ ID NOs: 313-350; (b) an HCVD of SEQ ID NO: 80, one of the sequences set forth in SEQ ID NOs: 88-128, one of the sequences set forth in SEQ ID NOs: 397-406, or one of the sequences set forth in SEQ ID NOs: 397-406; (c) an HCVD of SEQ ID NO: 156, one of the sequences set forth in SEQ ID NOs: 164-202, or one of the sequences set forth in SEQ ID NOs: 427-457; and (d) an HCVD of SEQ ID NO: 234 or one of the sequences set forth in SEQ ID NOs: 242-280; and/or the first ABD includes one of (a) to (d): (a) an LCVD of SEQ ID NO: 5, one of the sequences set forth in SEQ ID NOs: 53-79, or one of the sequences set forth in SEQ ID NOs: 351-396; (b) an LCVD of SEQ ID NO: 81, one of the sequences set forth in SEQ ID NOs: 129-155, or one of the sequences set forth in SEQ ID NOs: 407-426; (c) an LCVD of SEQ ID NO: 157, one of the sequences set forth in SEQ ID NOs: 203-233, or one of the sequences set forth in SEQ ID NOs: 458-492, and (d) an LCVD of SEQ ID NO: 235, or one of the sequences set forth in SEQ ID NOs: 281-312. In each of the foregoing embodiments, an alanine may be substituted where a histidine was already present.

In some embodiments, the first ABD includes one of (a) to (d): (a) an HCVD of SEQ ID NO: 4, one of the sequences set forth in SEQ ID NOs: 12-52, or one of the sequences set forth in SEQ LD NOs: 313-350, and/or an LCVD of SEQ ID NO: 5, one of the sequences set forth in SEQ ID NOs: 53-79, or one of the sequences set forth in SEQ ID NOs: 351-396, where the first ABD does not include (i) an HCVD of SEQ ID NO: 4 and an LCVD of SEQ ID NO: 5; (ii) an HCVD of SEQ ID NO: 4 and an LCVD that is not one of SEQ ID NOs: 57, 58, 59, 61, 62, 64, 65, 66, 67, 71, 74, 75, 76, 77, 78, 79, 351, 352, 353, 354, 355, 359, 360, 361, 362, 365, 366, 367, 368, 370, 371, 372, 373, 376, 377, 383, 386, 387, 388, 389, 392, 393, 394, 395, and 396, or (iii) an LCVD of SEQ ID NO: 5 and an HCVD that is not one of SEQ ID NOs: 15, 18, 19, 22, 24, 25, 32, 33, 34, 35, 36, 37, 38, 41, 43, 46, 47, 50, 318, 320, 322, 323, 324, 325, 328, 332, 333, 334, 336, 337, 343, 344, 345, 347, and 348; (b) an HCVD of SEQ ID NO: 80, one of the sequences set forth in SEQ ID NOs: 88-128, or one of the sequences set forth in SEQ ID NOs: 397-406; and/or an LCVD of SEQ ID NO: 81, one of the sequences set forth in SEQ ID NOs: 129-155, or one of the sequences set forth in SEQ ID NOs: 407-426, where the first ABD does not include (i) an HCVD of SEQ ID NO: 80 and an LCVD of SEQ ID NO: 81; (ii) an HCVD of SEQ ID NO: 80 and an LCVD that is not one of SEQ ID NOs: 131, 133, 134, 135, 136, 138, 139, 140, 141, 142, 143, 144, 147, 151, 152, 153, 411, 412, 417, 418, and 423; or (iii) an LCVD of SEQ ID NO: 81 and an HCVD that is not one of SEQ ID NOs: 91, 101, 103, 112, 113, 116, 119, 122, 123, 125, and 126; (c) an HCVD of SEQ ID NO: 156, or one of the sequences set forth in SEQ ID NOs: 164-202; and/or an LCVD of SEQ ID NO: 157, one of the sequences set forth in SEQ ID NOs: 203-233, or one of the sequences set forth in SEQ ID NOs: 458-492, where the first ABD does not include (i) an HCVD of SEQ ID NO: 156 and an LCVD of SEQ ID NO: 157; (ii) an HCVD of SEQ ID NO: 156 and an LCVD that is not one of the sequences set forth in SEQ ID NOs: 204, 211, 213, 215, 218, 220, 221, 222, 223, 224, 225, 228, 229, 231, 232, 233, 459, 460, 462, 464, 465, 466, 467, 468, 469, 470, 473, 474, 485, 488, 489, and 491; or (iii) an LCVD of SEQ ID NO: 157 and an HCVD that is not one of the sequences set forth in SEQ ID NOs: 164, 167, 168, 171, 172, 175, 178, 180, 181, 183, 196, 197, 200, 201, 427, 428, 429, 430, 431, 433, 434, 435, 436, 437, 438, 439, 440, 441, 443, 444, 445, 446, 447, 449, 451, 454, 456, and 457; and (d) an HCVD of SEQ ID NO: 234 or one of the sequences set forth in SEQ ID NOs: 242-280; and/or an LCVD of SEQ ID NO: 235 or one of the sequences set forth in SEQ ID NOs: 281-312, where the first ABD does not include an HCVD of SEQ ID NO: 234 and an LCVD of SEQ ID NO: 235. In each of the foregoing embodiments, an alanine may be substituted where a histidine was already present.

In some embodiments, the ABPC is degraded in the target mammalian cell following internalization of the ABPC by the target mammalian cell. In some embodiments, the ABPC includes a conjugated toxin, radioisotope, drug, or small molecule.

In some embodiments, the composition provides for an increase in toxin liberation in the target mammalian cell as compared to a composition including the same amount of a control ABPC. In some embodiments, the composition provides for at least a 20% increase in toxin liberation in the target mammalian cell as compared to a composition including the same amount of a control ABPC. In some embodiments, the composition provides for at least a 50% increase in toxin liberation in the target mammalian cell as compared to a composition including the same amount of a control ABPC. In some embodiments, the composition provides for at least a 2-fold increase in toxin liberation in the target mammalian cell as compared to a composition including the same amount of a control ABPC. In some embodiments, the composition provides for at least a 5-fold increase in toxin liberation in the target mammalian cell as compared to a composition including the same amount of a control ABPC.

In some embodiments, the composition provides for an increase in target mammalian cell killing as compared to a composition including the same amount of a control ABPC. In some embodiments, the composition provides for at least a 20% or 50% increase in target mammalian cell killing as compared to a composition including the same amount of a control ABPC. In some embodiments, the composition provides for at least a 2- or 5-fold increase in target mammalian cell killing as compared to a composition including the same amount of a control ABPC.

In some embodiments, the composition provides for an increase in endolysosomal delivery in the target mammalian cell as compared to a composition including the same amount of a control ABPC. In some embodiments, the composition provides for at least a 20% or 50% increase in endolysosomal delivery in the target mammalian cell as compared to a composition including the same amount of a control ABPC. In some embodiments, the composition provides for at least a 2- or 5-fold increase in endolysosomal delivery in the target mammalian cell as compared to a composition including the same amount of a control ABPC.

In some embodiments, the composition results in a less of a reduction in the level of TROP2 presented on the surface of the target mammalian cell as compared to a composition including the same amount of a control ABPC. In some embodiments, the composition does not result in a detectable reduction in the level of the TROP2 presented on the surface of the target mammalian cell.

Also provided herein are pharmaceutical compositions including an effective amount of an antigen-binding protein construct (ABPC) including: a first ABD that is capable of specifically binding TROP2 or an epitope of TROP2 presented on the surface of a target mammalian cell; and a conjugated toxin, radioisotope, drug, or small molecule, where: (a) the dissociation rate of the first ABD at a pH of about 4.0 to about 6.5 is faster than the dissociation rate at a pH of about 7.0 to about 8.0; or the dissociation constant (KD) of the first ABD at a pH of about 4.0 to about 6.5 is greater than the KD at a pH of about 7.0 to about 8.0; and (b) the composition provides for one or more of: an increase in toxin liberation in the target mammalian cell as compared to a composition including the same amount of a control ABPC; an increase in target mammalian cell killing as compared to a composition including the same amount of a control ABPC; and an increase in endolysosomal delivery in the target mammalian cell as compared to a composition including the same amount of a control ABPC.

In some embodiments, the first ABD includes an HCVD that is one of (a) to (d): (a) an HCVD of sacituzumab; (b) an HCVD of datopotamab; (c) an HCVD of hu7E6SVG; and (d) an HCVD of A1,2X4, each HCVD with one or more histidine substitution. In some embodiments, the first ABD includes an LCVD of one of (a) to (d): (a) an LCVD of sacituzumab; (b) an LCVD of datopotamab; (c) an LCVD of hu7E6SVG; and (d) an LCVD of A1,2X4, each LCVD with one or more histidine substitution. In each of the foregoing embodiments, an alanine may be substituted where a histidine was already present.

In some embodiments, the first ABD includes one of (a) to (d): (a) an HCVD of sacituzumab and/or an LCVD of sacituzumab; (b) an HCVD of datopotamab and/or an LCVD of datopotamab; (c) an HCVD of hu7E6SVG and/or an LCVD of hu7E6SVG; and (d) an HCVD of A1,2X4 and/or an LCVD of A1,2X4, each HCVD and/or LCVD with one or more histidine substitution. In each of the foregoing embodiments, an alanine may be substituted where a histidine was already present.

In some embodiments, the HCVD includes one of (a) to (d): (a) an HCVD of sacituzumab including SEQ ID NO: 4; (b) an HCVD of datopotamab including SEQ ID NO: 80; (c) an HCVD of hu7E6SVG including SEQ ID NO: 156; and (d) an HCVD of A1,2X4 including SEQ ID NO: 234. In some embodiments, the LCVD includes one of (a) to (d): (a) an LCVD of sacituzumab including SEQ ID NO: 5; (b) an LCVD of datopotamab including SEQ ID NO: 81; (c) an LCVD of hu7E6SVG including SEQ ID NO: 157; and (d) an LCVD of A1,2X4 including SEQ ID NO: 235. In each of the foregoing embodiments, an alanine may be substituted where a histidine was already present.

In some embodiments, the first ABD includes an HCVD including one of (a) to (d): (a) an HCVD including a CDR1, a CDR2, and a CDR3 of SEQ ID NOs: 6, 7, and 8, respectively, with collectively a total of one or more amino acid positions in SEQ ID NOs: 6, 7, and 8 substituted with a histidine; (b) an HCVD including a CDR1, a CDR2, and a CDR3 of SEQ ID NOs: 82, 83, and 84, respectively, with collectively a total of one or more amino acid positions in SEQ ID NOs: 82, 83, and 84 substituted with a histidine; (c) an HCVD including a CDR1, a CDR2, and a CDR3 of SEQ ID NOs: 158, 159, and 160, respectively, with collectively a total of one or more amino acid positions in SEQ ID NOs: 158, 159, and 160 substituted with a histidine; and (d) an HCVD including a CDR1, a CDR2, and a CDR3 of SEQ ID NOs: 236, 237, and 238, respectively, with collectively a total of one or more amino acid positions in SEQ ID NOs: 236, 237, and 238 substituted with a histidine. In each of the foregoing embodiments, an alanine may be substituted where a histidine was already present.

In some embodiments, the first ABD includes an LCVD of one of (a) to (d): (a) an LCVD including a CDR1, a CDR2, and a CDR3 of SEQ ID NOs: 9, 10, and 11, respectively, with collectively a total of one or more amino acid positions in SEQ ID NOs: 9, 10, and 11 substituted with a histidine; (b) an LCVD including a CDR1, a CDR2, and a CDR3 of SEQ ID NOs: 85, 86, and 87, respectively, with collectively a total of one or more amino acid positions in SEQ ID NOs: 85, 86, and 87 substituted with a histidine; (c) an LCVD including a CDR1, a CDR2, and a CDR3 of SEQ ID NOs: 161, 162, and 163, respectively, with collectively a total of one or more amino acid positions in SEQ ID NOs: 161, 162, and 163 substituted with a histidine; and (d) an LCVD including a CDR1, a CDR2, and a CDR3 of SEQ ID NOs: 239, 240, and 241, respectively, with collectively a total of one or more amino acid positions in SEQ ID NOs: 239, 240, and 241 substituted with a histidine. In each of the foregoing embodiments, an alanine may be substituted where a histidine was already present.

In some embodiments, the first ABD includes one of (a) to (d): (a) an HCVD including a CDR1, a CDR2, and a CDR3 of SEQ ID NOs: 6, 7, and 8, respectively, with collectively a total of one or more amino acid positions in SEQ ID NOs: 6, 7, and 8 substituted with a histidine; and/or an LCVD including a CDR1, a CDR2, and a CDR3 of SEQ ID NOs: 9, 10, and 11, respectively, with collectively a total of one or more amino acid positions in SEQ ID NOs: 9, 10, and 11 substituted with a histidine; (b) an HCVD including a CDR1, a CDR2, and a CDR3 of SEQ ID NOs: 82, 83, and 84, respectively, with collectively a total of one or more amino acid positions in SEQ ID NOs: 82, 83, and 84 substituted with a histidine; and/or an LCVD including a CDR1, a CDR2, and a CDR3 of SEQ ID NOs: 85, 86, and 87, respectively, with collectively a total of one or more amino acid positions in SEQ ID NOs: 85, 86, and 87 substituted with a histidine, (c) an HCVD including a CDR1, a CDR2, and a CDR3 of SEQ ID NOs: 158, 159, and 160, respectively, with collectively a total of one or more amino acid positions in SEQ ID NOs: 158, 159, and 160 substituted with a histidine; and/or an LCVD including a CDR1, a CDR2, and a CDR3 of SEQ ID NOs: 161, 162, and 163, respectively, with collectively a total of one or more amino acid positions in SEQ ID NOs: 161, 162, and 163 substituted with a histidine; and (d) an HCVD including a CDR1, a CDR2, and a CDR3 of SEQ ID NOs: 236, 237, and 238, respectively, with collectively a total of one or more amino acid positions in SEQ ID NOs: 236, 237, and 238 substituted with a histidine; and/or an LCVD including a CDR1, a CDR2, and a CDR3 of SEQ ID NOs: 239, 240, and 241, respectively, with collectively a total of one or more amino acid positions in SEQ ID NOs: 239, 240, and 241 substituted with a histidine. In each of the foregoing embodiments, an alanine may be substituted where a histidine was already present.

In some embodiments, the first ABD includes an HCVD that is one of (a) to (d): (a) an HCVD that is at least 90%, 95%, or 98% identical to SEQ ID NO: 4, where the HCVD includes a histidine at one or more positions in SEQ ID NO: 4 selected from: 29, 32, 33, 50, 52, 53, 60, 61, 62, 63, 64, 65, 66, 99, 101, 104, 105, and 108; (b) an HCVD that is at least 90%, 95%, or 98% identical to SEQ ID NO: 80, where the HCVD includes a histidine at one or more positions in SEQ ID NO: 80 selected from: 29, 53, 55, 64, 65, 98, 101, 104, 105, 107, and 108; (c) an HCVD that is at least 90%, 95%, or 98% identical to SEQ ID NO: 156, where the HCVD includes a histidine at one or more positions in SEQ ID NO: 156 selected from: 26, 29, 30, 33, 34, 51, 54, 56, 57, 59, 102, 103, and 106; and (d) an HCVD that is at least 90%, 95%, or 98% identical to SEQ ID NO: 234, where the HCVD includes a histidine at one or more positions in SEQ ID NO: 234. In each of the foregoing embodiments, an alanine may be substituted where a histidine was already present.

In some embodiments, the first ABD includes an LCVD of one of (a) to (d): (a) an LCVD that is at least 90%, 95%, or 98% identical to SEQ ID NO: 5, where the LCVD includes a histidine at one or more positions in SEQ ID NO: 5 selected from: 28, 29, 30, 31, 32, 33, 50, 51, 52, 53, 89, 92, 93, 94, 95, 96, and 97; (b) an LCVD that is at least 90%, 95%, or 98% identical to SEQ ID NO: 81, where the LCVD includes a histidine at one or more positions in SEQ ID NO: 81 selected from: 26, 28, 29, 30, 31, 33, 34, 50, 51, 52, 53, 54, 89, 93, 94, and 95; (c) an LCVD that is at least 90%, 95%, or 98% identical to SEQ ID NO: 157, where the LCVD includes a histidine at one or more positions in SEQ ID NO: 157 selected from 25, 32, 34, 36, 54, 56, 57, 58, 59, 60, 93, 96, 97, 99, 100, and 101; and (d) an LCVD that is at least 90%, 95%, or 98% identical to SEQ ID NO: 235, where the LCVD includes a histidine at one or more positions in SEQ ID NO: 235. In each of the foregoing embodiments, an alanine may be substituted where a histidine was already present.

In some embodiments, the first ABD includes an HCVD that is one of (a) to (d): (a) an HCVD that is at least 90%, 95%, or 98% identical to SEQ ID NO: 4, where the HCVD includes a histidine at two or more positions in SEQ ID NO: 4 selected from: 29, 32, 33, 50, 52, 53, 60, 61, 62, 63, 64, 65, 66, 99, 101, 104, 105, and 108; (b) an HCVD that is at least 90%, 95%, or 98% identical to SEQ ID NO: 80, where the HCVD includes a histidine at two or more positions in SEQ ID NO: 80 selected from: 29, 53, 55, 64, 65, 98, 101, 104, 105, 107, and 108; (c) an HCVD that is at least 90%, 95%, or 98% identical to SEQ ID NO: 156, where the HCVD includes a histidine at two or more positions in SEQ ID NO: 156 selected from: 26, 29, 30, 33, 34, 51, 54, 56, 57, 59, 102, 103, and 106, and (d) an HCVD that is at least 90%, 95%, or 98% identical to SEQ ID NO: 234, where the HCVD includes a histidine at two or more positions in SEQ ID NO: 234. In each of the foregoing embodiments, an alanine may be substituted where a histidine was already present.

In some embodiments, the first ABD includes an LCVD of one of (a) to (d): (a) an LCVD that is at least 90%, 95%, or 98% identical to SEQ ID NO: 5, where the LCVD includes a histidine at two or more positions in SEQ ID NO: 5 selected from: 28, 29, 30, 31, 32, 33, 50, 51, 52, 53, 89, 92, 93, 94, 95, 96, and 97; (b) an LCVD that is at least 90%, 95%, or 98% identical to SEQ ID NO: 81, where the LCVD includes a histidine at two or more positions in SEQ ID NO: 81 from: 26, 28, 29, 30, 31, 33, 34, 50, 51, 52, 53, 54, 89, 93, 94, and 95; (c) an LCVD that is at least 90%, 95%, or 98% identical to SEQ ID NO: 157, where the LCVD includes a histidine at two or more positions in SEQ ID NO: 157 selected from: 25, 32, 34, 36, 54, 56, 57, 58, 59, 60, 93, 96, 97, 99, 100, and 101; and (d) an LCVD that is at least 90%, 95%, or 98% identical to SEQ ID NO: 235, where the LCVD includes a histidine at two or more positions in SEQ ID NO: 235. In each of the foregoing embodiments, an alanine may be substituted where a histidine was already present.

In some embodiments, the first ABD includes one of (a) to (d): (a) an HCVD that is at least 90%, 95%, or 98% identical to SEQ ID NO: 4, where the HCVD includes a histidine at one or more positions in SEQ ID NO: 4 selected from: 29, 32, 33, 50, 52, 53, 60, 61, 62, 63, 64, 65, 66, 99, 101, 104, 105, and 108; and/or an LCVD that is at least 90%, 95%, or 98% identical to SEQ ID NO: 5, where the LCVD includes a histidine at one or more positions in SEQ ID NO: 5 selected from: 28, 29, 30, 31, 32, 33, 50, 51, 52, 53, 89, 92, 93, 94, 95, 96, and 97; (b) an HCVD that is at least 90%, 95%, or 98% identical to SEQ ID NO: 80, where the HCVD includes a histidine at one or more positions in SEQ ID NO: 80 selected from: 29, 53, 55, 64, 65, 98, 101, 104, 105, 107, and 108; and/or an LCVD that is at least 90%, 95%, or 98% identical to SEQ ID NO: 81, where the LCVD includes a histidine at one or more positions in SEQ ID NO: 81 selected from: 26, 28, 29, 30, 31, 33, 34, 50, 51, 52, 53, 54, 89, 93, 94, and 95; (c) an HCVD that is at least 90%, 95%, or 98% identical to SEQ ID NO: 156, where the HCVD includes a histidine at one or more positions in SEQ ID NO: 156 selected from: 26, 29, 30, 33, 34, 51, 54, 56, 57, 59, 102, 103, and 106, and/or an LCVD that is at least 90%, 95%, or 98% identical to SEQ ID NO: 157, where the LCVD includes a histidine at one or more positions in SEQ ID NO: 157 selected from 25, 32, 34, 36, 54, 56, 57, 58, 59, 60, 93, 96, 97, 99, 100, and 101; and (d) an HCVD that is at least 90%, 95%, or 98% identical to SEQ ID NO: 234, where the HCVD includes a histidine at one or more positions in SEQ ID NO: 234; and/or an LCVD that is at least 90%, 95%, or 98% identical to SEQ ID NO: 235, where the LCVD includes a histidine at one or more positions in SEQ ID NO: 235. In each of the foregoing embodiments, an alanine may be substituted where a histidine was already present.

In some embodiments, the first ABD includes an HCVD that is one of (a) to (d): (a) an HCVD of SEQ ID NO: 4, one of the sequences set forth in SEQ ID NOs: 12-52, or one of the sequences set forth in SEQ ID NOs: 313-350; (b) an HCVD of SEQ ID NO: 80, one of the sequences set forth in SEQ ID NOs: 88-128, one of the sequences set forth in SEQ ID NOs: 397-406, or one of the sequences set forth in SEQ ID NOs: 397-406; (c) an HCVD of SEQ ID NO: 156, one of the sequences set forth in SEQ ID NOs: 164-202, or one of the sequences set forth in SEQ ID NOs: 427-457; and (d) an HCVD of SEQ ID NO: 234, or one of the sequences set forth in SEQ ID NOs: 242-280. In some embodiments, the first ABD includes an LCVD of one of (a) to (d): (a) an LCVD of SEQ ID NO: 5, one of the sequences set forth in SEQ ID NOs: 53-79, or one of the sequences set forth in SEQ ID NOs: 351-396; (b) an LCVD of SEQ ID NO: 81, one of the sequences set forth in SEQ ID NOs: 129-155, or one of the sequences set forth in SEQ ID NOs: 407-426; (c) an LCVD of SEQ ID NO: 157, one of the sequences set forth in SEQ ID NOs: 203-233, or one of the sequences set forth in SEQ ID NOs: 458-492; and (d) an LCVD of SEQ ID NO: 235, or one of the sequences set forth in SEQ ID NOs: 281-312.

In some embodiments, the first ABD includes one of (a) to (d): (a) an HCVD of SEQ ID NO: 4, one of the sequences set forth in SEQ ID NOs: 12-52, or one of the sequences set forth in SEQ ID NOs: 313-350, and/or an LCVD of SEQ ID NO: 5, one of the sequences set forth in SEQ ID NOs: 53-79, or one of the sequences set forth in SEQ ID NOs: 351-396, where the first ABD does not include (i) an HCVD of SEQ ID NO: 4 and an LCVD of SEQ ID NO: 5; (ii) an HCVD of SEQ ID NO: 4 and an LCVD that is not one of SEQ ID NOs: 57, 58, 59, 61, 62, 64, 65, 66, 67, 71, 74, 75, 76, 77, 78, 79, 351, 352, 353, 354, 355, 359, 360, 361, 362, 365, 366, 367, 368, 370, 371, 372, 373, 376, 377, 383, 386, 387, 388, 389, 392, 393, 394, 395, and 396; or (iii) an HCVD that is not one of SEQ ID NOs: 15, 18, 19, 22, 24, 25, 32, 33, 34, 35, 36, 37, 38, 41, 43, 46, 47, 50, 318, 320, 322, 323, 324, 325, 328, 332, 333, 334, 336, 337, 343, 344, 345, 347, and 348 and an LCVD of SEQ ID NO: 5; (b) an HCVD of SEQ ID NO: 80, one of the sequences set forth in SEQ ID NOs: 88-128, one of the sequences set forth in SEQ ID NOs: 397-406, or one of the sequences set forth in SEQ ID NOs: 397-406; and/or an LCVD of SEQ ID NO: 81, one of the sequences set forth in SEQ ID NOs: 129-155, or one of the sequences set forth in SEQ ID NOs: 407-426, where the first ABD does not include (i) an HCVD of SEQ ID NO: 80 and an LCVD of SEQ ID NO: 81; (ii) an HCVD of SEQ ID NO: 80 and an LCVD that is not one of SEQ ID NOs: 131, 133, 134, 135, 136, 138, 139, 140, 141, 142, 143, 144, 147, 151, 152, 153, 411, 412, 417, 418, and 423; or (iii) an HCVD that is not one of SEQ ID NOs: 91, 101, 103, 112, 113, 116, 119, 122, 123, 125, and 126 and an LCVD of SEQ ID NO: 81; (c) an HCVD of SEQ ID NO: 156, one of the sequences set forth in SEQ ID NOs: 164-202, or one of the sequences set forth in SEQ ID NOs: 427-457; and/or an LCVD of SEQ ID NO: 157, one of the sequences set forth in SEQ ID NOs: 203-233, or one of the sequences set forth in SEQ ID NOs: 458-492, where the first ABD does not include (i) an HCVD of SEQ ID NO: 156 and an LCVD of SEQ ID NO: 157; (ii) an HCVD of SEQ ID NO: 156 and an LCVD that is not one of the sequences set forth in SEQ ID NOs: 204, 211, 213, 215, 218, 220, 221, 222, 223, 224, 225, 228, 229, 231, 232, 233, 459, 460, 462, 464, 465, 466, 467, 468, 469, 470, 473, 474, 485, 488, 489, and 491; or (iii) an HCVD that is not one of the sequences set forth in SEQ ID NOs: 164, 167, 168, 171, 172, 175, 178, 180, 181, 183, 196, 197, 200, 201, 427, 428, 429, 430, 431, 433, 434, 435, 436, 437, 438, 439, 440, 441, 443, 444, 445, 446, 447, 449, 451, 454, 456, and 457 and an LCVD of SEQ ID NO: 157; and (d) an HCVD of SEQ ID NO: 234, or one of the sequences set forth in SEQ ID NOs: 242-280; and/or an LCVD of SEQ ID NO: 235, or one of the sequences set forth in SEQ ID NOs: 281-312, where the first ABD does not include an HCVD of SEQ ID NO: 234 and an LCVD of SEQ ID NO: 235.

In some embodiments, the composition provides for an increase in toxin liberation in the target mammalian cell as compared to a composition including the same amount of a control ABPC. In some embodiments, the composition provides for at least a 20% or 50% increase in toxin liberation in the target mammalian cell as compared to a composition including the same amount of a control ABPC. In some embodiments, the composition provides for at least a 2- or 5-fold increase in toxin liberation in the target mammalian cell as compared to a composition including the same amount of a control ABPC.

In some embodiments, the composition provides for an increase in target mammalian cell killing as compared to a composition including the same amount of a control ABPC. In some embodiments, the composition provides for at least a 20% or 50% increase in target mammalian cell killing as compared to a composition including the same amount of a control ABPC. In some embodiments, the composition provides for at least a 2- or 5-fold increase in target mammalian cell killing as compared to a composition including the same amount of a control ABPC.

In some embodiments, the composition provides for an increase in endolysosomal delivery, including at least a 20% or 50% increase in endolysosomal delivery as compared to a composition including the same amount of a control ABPC. In some embodiments, the composition provides for at least a 2- or 5-fold increase in endolysosomal delivery in the target mammalian cell as compared to a composition including the same amount of a control ABPC.

In some embodiments, the composition results in a less of a reduction in the level of TROP2 presented on the surface of the target mammalian cell as compared to a composition including the same amount of a control ABPC. In some embodiments, the composition does not result in a detectable reduction in the level of the TROP2 presented on the surface of the target mammalian cell. In some embodiments, the target mammalian cell is a cancer cell.

In some embodiments, the dissociation rate of the ABD at a pH of ~4.0 to ~6.5 is at least 10% faster than the dissociation rate of the ABD at a pH of ~7.0 to ~8.0. In some embodiments, the dissociation rate of the ABD at a pH of ~4.0 to ~6.5 is at least 3~fold faster than the dissociation rate of the ABD at a pH of ~7.0 to ~8.0. In some embodiments, the dissociation rate of the ABD at a pH of ~4.0 to ~6.5 is at least 10~fold faster than the dissociation rate of the ABD at a pH of ~7.0 to ~8.0. In some embodiments, the K_{D} of the ABD at a pH of ~4.0 to ~6.5 is at least 10% greater than the KT of the ABD at a pH of ~7.0 to ~8.0. In some embodiments, the K_{D} of the ABD at a pH of ~4.0 to ~6.5 is at least 3~fold greater than the K_{D} of the ABD at a pH of ~7.0 to ~8.0. In some embodiments, the K_{D} of the ABD at a pH of ~4.0 to ~6.5 is at least 10~fold greater than the K_{D} of the ABD at a pH of ~7.0 to ~8.0.

In some embodiments, the ABPC is cytotoxic or cytostatic to the target mammalian cell.

In some embodiments, the ABPC is cross-reactive with anon-human primate TROP2 and human TROP2. In some embodiments, the ABPC is cross-reactive with a non-human primate TROP2, a human TROP2, and one or both of rat TROP2 and a mouse TROP2. In some embodiments, the ABPC is cross-reactive with a non-human primate TROP2, a human TROP2, a rat TROP2, and a mouse TROP2. In some embodiments, the ABD binds to an epitope of TROP2 that is present on the surface of cells from an Old World Monkey.

In some embodiments, the ABPC includes a single polypeptide. In some embodiments, the ABD is selected from a VH domain, a VHH domain, a VNAR domain, and a scFv. In some embodiments, the ABPC is a BiTe, a (scFv)₂, a nanobody, a nanobody-HSA, a DART, a TandAb, a scDiabody, a scDiabody-CH3, scFv-CH-CL-scFv, a HSAbody, scDiabody-HSA, or a tandem-scFv

In some embodiments, the ABPC includes two or more polypeptides. In some embodiments, the ABPC is selected from the group of an antibody, a VHH-scAb, a VHH-Fab, a Dual scFab, a F(ab′)₂, a diabody, a crossMab, a DAF (two-in-one), a DAF (four-in-one), a DutaMab, a DT-IgG, a knobs-in-holes common light chain, a knobs-in-holes assembly, a charge pair, a Fab-arm exchange, a SEEDbody, a LUZ-Y, a Fcab, a κλ-body, an orthogonal Fab, a DVD-IgG, a IgG(H)-scFv, a scFv-(H)IgG, IgG(L)-scFv, scFv-(L)IgG, IgG(L,H)-Fv, IgG(H)-V, V(H)-IgG, IgG(L)-V, V(L)-IgG, KIH IgG-scFab, 2scFv-IgG, IgG-2scFv, scFv4-Ig, Zybody, DVI-IgG, Diabody-CH3, a triple body, a miniantibody, a minibody, a TriBi minibody, scFv-CH3 KIH, Fab-scFv, a F(ab′)₂-scFv2, a scFv-KIH, a Fab-scFv-Fc, a tetravalent HCAb, a scDiabody-Fc, a Diabody-Fc, a tandem scFv-Fc, a VHH-Fc, a tandem VHH-Fc, a VHH-Fc KiH, a Fab-VHH-Fc, an Intrabody, a dock and lock, an ImmTAC, an IgG-IgG conjugate, a Cov-X-Body, a scFv1-PEG-scFv2, an Adnectin, a DARPin, a fibronectin, a DEP conjugate, a PROTAB, and a PROTAC.

In some embodiments, the ABPC or ABD includes a proteolysis-targeting antibody (PROTAB) (as described, e.g., in Marei et al, “Antibody targeting of E3 ubiquitin ligases for receptor degradation. Nature, 6 Oct. 2022). In some embodiments, a PROTAB or PROTAC may tether cell-surface E3 ubiquitin ligases to transmembrane proteins, resulting in target degradation both in vitro and in vivo.

In some embodiments, at least one polypeptide of the ABPC is conjugated to the toxin, the radioisotope, the drug, or the small molecule via a cleavable linker. In some embodiments, at least one polypeptide of the ABPC is conjugated to the toxin, the radioisotope, the drug, or the small molecule via a non-cleavable linker.

In some embodiments, the half-life of the ABPC in vivo is increased as compared to the half-life of a control ABPC in vivo. In some embodiments, the half-life of the ABPC in vivo is increased about 5% to about 95% as compared to the half-life of a control ABPC in vivo. In some embodiments, the half-life of the ABPC in vivo is increased about 10% to about 95% as compared to the half-life of a control ABPC in vivo. In some embodiments, the half-life of the ABPC in vivo is increased about 30% to about 95% as compared to the half-life of a control ABPC in vivo. In some embodiments, the half-life of the ABPC in vivo is increased about 50% to about 95% as compared to the half-life of a control ABPC in vivo. In some embodiments, the half-life of the ABPC in vivo is increased about 70% to about 95% as compared to the half-life of a control ABPC in vivo. In other embodiments, the in vivo half-life may be decreased.

In some embodiments, the control ABPC is capable of specifically binding to TROP2 or an epitope of TROP2 presented on the surface of a target mammalian cell, where: (a) the control ABPC includes a first ABD: (b) the dissociation rate of the first ABD of the control ABPC at a pH of ~4.0 to ~6.5 is no more than 3-fold faster than the dissociation rate at a pH of ~7.0 to ~8.0; and (c) the dissociation constant (K_{D}) of the first ABD of the control ABPC at a pH of ~4.0 to ~6.5 is no more than 3-fold greater than the K_{D} at a pH of ~7.0 to ~8.0.

In some embodiments, the control ABPC is capable of specifically binding to TROP2 or an epitope of TROP2 presented on the surface of a target mammalian cell, where: (a) the control ABPC includes a first ABD; (b) the dissociation rate of the first ABD of the control ABPC at a pH of ~4.0 to ~6.5 is no more than 2-fold faster than the dissociation rate at a pH of ~7.0 to ~8.0; and (c) the dissociation constant (K_{D}) of the first ABD of the control ABPC at a pH of ~4.0 to ~6.5 is no more than 2-fold greater than the K_{D} at a pH of ~7.0 to ~8.0.

In some embodiments, the control ABPC is capable of specifically binding to TROP2 or an epitope of TROP2 presented on the surface of a target mammalian cell, where: (a) the control ABPC includes a first ABD; (b) the dissociation rate of the first ABD of the control ABPC at a pH of ~4.0 to ~6.5 is no more than 1-fold faster than the dissociation rate at a pH of ~7.0 to ~8.0; and (c) the dissociation constant (K_{D}) of the first ABD of the control ABPC at a pH of ~4.0 to ~6.5 is no more than 1-fold greater than the Kr at a pH of ~7.0 to ~8.0.

In some embodiments, the control ABPC is selected from one of: (a) sacituzumab; (b) datopotamab; (b) hu7E6SVG; and (d) A1,2X4.

In some embodiments, the ABPC includes a second ABD.

Also provided herein are kits including at least one dose of any of the pharmaceutical compositions described herein.

Also provided herein are antigen-binding protein constructs (ABPCs) including: a first ABD that is capable of specifically binding TROP2 or an epitope of TROP2 presented on the surface of a target mammalian cell, where: (a) the dissociation rate of the first ABD at a pH of about 4.0 to about 6.5 is faster than the dissociation rate at a pH of about 7.0 to about 8.0; or (b) the dissociation constant (K_{D}) of the first ABD at a pH of about 4.0 to about 6.5 is greater than the K_{D} at a pH of about 7.0 to about 8.0.

In some embodiments, the first ABD includes one of (a) to (d): (a) an HCVD of sacituzumab with one or more amino acids substituted with a histidine; (b) an HCVD of datopotamab with one or more amino acids substituted with a histidine; (c) an HCVD of hu7E6SVG with one or more amino acids substituted with a histidine; and (d) an HCVD of A1,2X4 with one or more amino acids substituted with a histidine. In some embodiments, the first ABD includes one of (a) to (d): (a) an LCVD of sacituzumab with one or more amino acids substituted with a histidine; (b) an LCVD of datopotamab with one or more amino acids substituted with a histidine; (c) an LCVD of hu7E6SVG with one or more amino acids substituted with a histidine; and (d) an LCVD of A1,2X4 with one or more amino acids substituted with a histidine. In each of the foregoing embodiments, an alanine may be substituted where a histidine was already present.

In some embodiments, the first ABD includes one of (a) to (d): (a) an HCVD of sacituzumab and/or an LCVD of sacituzumab, either or both with one or more amino acids substituted with a histidine; (b) an HCVD of datopotamab and/or an LCVD of datopotamab, either or both with one or more amino acids substituted with a histidine; (c) an HCVD of hu7E6SVG and/or an LCVD of hu7E6SVG, either or both with one or more amino acids substituted with a histidine; and (d) an HCVD of A1,2X4 and/or an LCVD of A1,2X4, either or both with one or more amino acids substituted with a histidine. In some embodiments, the HCVD includes one of (a) to (d): (a) an HCVD of sacituzumab including SEQ ID NO: 4; (b) an HCVD of datopotamab including SEQ ID NO: 80; (c) an HCVD of hu7E6SVG including SEQ ID NO: 156; and (d) an HCVD of A1,2X4 including SEQ ID NO: 234. In some embodiments, the LCVD includes one of (a) to (d): (a) an LCVD of sacituzumab including SEQ ID NO: 5; (b) an LCVD of datopotamab including SEQ ID NO: 81; (c) an LCVD of hu7E6SVG including SEQ ID NO: 157; and (d) an LCVD of A1,2X4 including SEQ ID NO: 235. In each of the foregoing embodiments, an alanine may be substituted where a histidine was already present.

In some embodiments, the first ABD includes an HCVD that is one of (a) to (d): (a) an HCVD including a CDR1, a CDR2, and a CDR3 of SEQ ID NOs: 6, 7, and 8, respectively, with collectively a total of one or more amino acid positions in SEQ ID NOs: 6, 7, and 8 substituted with a histidine; (b) an HCVD including a CDR1, a CDR2, and a CDR3 of SEQ ID NOs: 82, 83, and 84, respectively, with collectively a total of one or more amino acid positions in SEQ ID NOs: 82, 83, and 84 substituted with a histidine; (c) an HCVD including a CDR1, a CDR2, and a CDR3 of SEQ ID NOs: 158, 159, and 160, respectively, with collectively a total of one or more amino acid positions in SEQ ID NOs: 158, 159, and 160 substituted with a histidine; and (d) an HCVD including a CDR1, a CDR2, and a CDR3 of SEQ ID NOs: 236, 237, and 238, respectively, with collectively a total of one or more amino acid positions in SEQ ID NOs: 236, 237, and 238 substituted with a histidine. In some embodiments, the first ABD includes an LCVD of one of (a) to (d): (a) an LCVD including a CDR1, a CDR2, and a CDR3 of SEQ ID NOs: 9, 10, and 11, respectively, with collectively a total of one or more amino acid positions in SEQ ID NOs: 9, 10, and 11 substituted with a histidine; (b) an LCVD including a CDR1, a CDR2, and a CDR3 of SEQ ID NOs: 85, 86, and 87, respectively, with collectively a total of one or more amino acid positions in SEQ ID NOs: 85, 86, and 87 substituted with a histidine; (c) an LCVD including a CDR1, a CDR2, and a CDR3 of SEQ ID NOs: 161, 162, and 163, respectively, with collectively a total of one or more amino acid positions in SEQ ID NOs: 161, 162, and 163 substituted with a histidine; and (d) an LCVD including a CDR1, a CDR2, and a CDR3 of SEQ ID NOs: 239, 240, and 241, respectively, with collectively a total of one or more amino acid positions in SEQ LD NOs: 239, 240, and 241 substituted with a histidine. In each of the foregoing embodiments, an alanine may be substituted where a histidine was already present.

In some embodiments, the first ABD includes one of (a) to (d): (a) an HCVD including a CDR1, a CDR2, and a CDR3 of SEQ ID NOs: 6, 7, and 8, respectively, with collectively a total of one or more amino acid positions in SEQ ID NOs: 6, 7, and 8 substituted with a histidine; and/or an LCVD including a CDR1, a CDR2, and a CDR3 of SEQ ID NOs: 9, 10, and 11, respectively, with collectively a total of one or more amino acid positions in SEQ ID NOs: 9, 10, and 11 substituted with a histidine; (b) an HCVD including a CDR1, a CDR2, and a CDR3 of SEQ ID NOs: 82, 83, and 84, respectively, with collectively a total of one or more amino acid positions in SEQ ID NOs: 82, 83, and 84 substituted with a histidine; and/or an LCVD including a CDR1, a CDR2, and a CDR3 of SEQ ID NOs: 85, 86, and 87, respectively, with collectively a total of one or more amino acid positions in SEQ ID NOs: 85, 86, and 87 substituted with a histidine, (c) an HCVD including a CDR1, a CDR2, and a CDR3 of SEQ ID NOs: 158, 159, and 160, respectively, with collectively a total of one or more amino acid positions in SEQ ID NOs: 158, 159, and 160 substituted with a histidine; and/or an LCVD including a CDR1, a CDR2, and a CDR3 of SEQ ID NOs: 161, 162, and 163, respectively, with collectively a total of one or more amino acid positions in SEQ ID NOs: 161, 162, and 163 substituted with a histidine; and (d) an HCVD including a CDR1, a CDR2, and a CDR3 of SEQ ID NOs: 236, 237, and 238, respectively, with collectively a total of one or more amino acid positions in SEQ ID NOs: 236, 237, and 238 substituted with a histidine; and/or an LCVD including a CDR1, a CDR2, and a CDR3 of SEQ ID NOs: 239, 240, and 241, respectively, with collectively a total of one or more amino acid positions in SEQ ID NOs: 239, 240, and 241 substituted with a histidine. In each of the foregoing embodiments, an alanine may be substituted where a histidine was already present.

In some embodiments, the first ABD includes an HCVD that is one of (a) to (d): (a) an HCVD that is at least 90%, 95%, or 98% identical to SEQ ID NO: 4, where the HCVD includes a histidine at one or more positions in SEQ ID NO: 4 selected from: 29, 32, 33, 50, 52, 53, 60, 61, 62, 63, 64, 65, 66, 99, 101, 104, 105, and 108; (b) an HCVD that is at least 90%, 95%, or 98% identical to SEQ ID NO: 80, where the HCVD includes a histidine at one or more positions in SEQ ID NO: 80 selected from: 29, 53, 55, 64, 65, 98, 101, 104, 105, 107, and 108; (c) an HCVD that is at least 90%/o, 95%, or 98% identical to SEQ ID NO: 156, where the HCVD includes a histidine at one or more positions in SEQ ID NO: 156 selected from: 26, 29, 30, 33, 34, 51, 54, 56, 57, 59, 102, 103, and 106; and (d) an HCVD that is at least 90%, 95%, or 98% identical to SEQ ID NO: 234, where the HCVD includes a histidine at one or more positions in SEQ ID NO: 234. In each of the foregoing embodiments, an alanine may be substituted where a histidine was already present.

In some embodiments, the first ABD includes an LCVD that is one of (a) to (d): (a) an LCVD that is at least 90%, 95%, or 98% identical to SEQ ID NO: 5, where the LCVD includes a histidine at one or more positions in SEQ ID NO: 5 selected from: 28, 29, 30, 31, 32, 33, 50, 51, 52, 53, 89, 92, 93, 94, 95, 96, and 97; (b) an LCVD that is at least 90%, 95%, or 98% identical to SEQ ID NO: 81, where the LCVD includes a histidine at one or more positions in SEQ ID NO: 81 selected from: 26, 28, 29, 30, 31, 33, 34, 50, 51, 52, 53, 54, 89, 93, 94, and 95; (c) an LCVD that is at least 90%, 95%, or 98% identical to SEQ ID NO: 157, where the LCVD includes a histidine at one or more positions in SEQ ID NO: 157 selected from 25, 32, 34, 36, 54, 56, 57, 58, 59, 60, 93, 96, 97, 99, 100, and 101; and (d) an LCVD that is at least 90%, 95%, or 98% identical to SEQ ID NO: 235, where the LCVD includes a histidine at one or more positions in SEQ ID NO: 235.

In some embodiments, the first ABD includes an HCVD that is one of (a) to (d): (a) an HCVD that is at least 90%, 95%, or 98% identical to SEQ ID NO: 4, where the HCVD includes a histidine at two or more positions in SEQ ID NO: 4 selected from: 29, 32, 33, 50, 52, 53, 60, 61, 62, 63, 64, 65, 66, 99, 101, 104, 105, and 108; (b) an HCVD that is at least 90%, 95%, or 98% identical to SEQ ID NO: 80, where the HCVD includes a histidine at two or more positions in SEQ ID NO: 80 selected from: 29, 53, 55, 64, 65, 98, 101, 104, 105, 107, and 108; (c) an HCVD that is at least 90%, 95%, or 98% identical to SEQ ID NO: 156, where the HCVD includes a histidine at two or more positions in SEQ ID NO: 156 selected from: 26, 29, 30, 33, 34, 51, 54, 56, 57, 59, 102, 103, and 106; and (d) an HCVD that is at least 90%, 95%, or 98% identical to SEQ ID NO: 234, where the HCVD includes a histidine at two or more positions in SEQ ID NO: 234. In each of the foregoing embodiments, an alanine may be substituted where a histidine was already present.

In some embodiments, the first ABD includes an LCVD that is one of (a) to (d): (a) an LCVD that is at least 90%/0, 95%, or 98% identical to SEQ ID NO: 5, where the LCVD includes a histidine at two or more positions in SEQ ID NO: 5 selected from: 28, 29, 30, 31, 32, 33, 50, 51, 52, 53, 89, 92, 93, 94, 95, 96, and 97; (b) an LCVD that is at least 90%, 95%, or 98% identical to SEQ ID NO: 81, where the LCVD includes a histidine at two or more positions in SEQ ID NO: 81 selected from: 26, 28, 29, 30, 31, 33, 34, 50, 51, 52, 53, 54, 89, 93, 94, and 95; (c) an LCVD that is at least 90%, 95%, or 98% identical to SEQ ID NO: 157, where the LCVD includes a histidine at two or more positions in SEQ ID NO: 157 selected from 25, 32, 34, 36, 54, 56, 57, 58, 59, 60, 93, 96, 97, 99, 100, and 101; and (d) an LCVD that is at least 90%, 95%, or 98% identical to SEQ ID NO: 235, where the LCVD includes a histidine at two or more positions in SEQ ID NO: 235. In each of the foregoing embodiments, an alanine may be substituted where a histidine was already present.

In some embodiments, the first ABD includes one of (a) to (d): (a) an HCVD that is at least 90%, 95%, or 98% identical to SEQ ID NO: 4, where the HCVD includes a histidine at one or more positions in SEQ ID NO: 4 selected from: 29, 32, 33, 50, 52, 53, 60, 61, 62, 63, 64, 65, 66, 99, 101, 104, 105, and 108; and/or an LCVD that is at least 90%, 95%, or 98% identical to SEQ ID NO: 5, where the LCVD includes a histidine at one or more positions in SEQ ID NO: 5 selected from: 28, 29, 30, 31, 32, 33, 50, 51, 52, 53, 89, 92, 93, 94, 95, 96, and 97; (b) an HCVD that is at least 90%, 95%, or 98% identical to SEQ ID NO: 80, where the HCVD includes a histidine at one or more positions in SEQ ID NO: 80 selected from: 29, 53, 55, 64, 65, 98, 101, 104, 105, 107, and 108; and/or an LCVD that is at least 90%, 95%, or 98% identical to SEQ ID NO: 81, where the LCVD includes a histidine at one or more positions in SEQ ID NO: 81 selected from: 26, 28, 29, 30, 31, 33, 34, 50, 51, 52, 53, 54, 89, 93, 94, and 95; (c) an HCVD that is at least 90%, 95%, or 98% identical to SEQ ID NO: 156, where the HCVD includes a histidine at one or more positions in SEQ ID NO: 156 selected from: 26, 29, 30, 33, 34, 51, 54, 56, 57, 59, 102, 103, and 106; and/or an LCVD that is at least 90%, 95%, or 98% identical to SEQ ID NO: 157, where the LCVD includes a histidine at one or more positions in SEQ ID NO: 157 selected from 25, 32, 34, 36, 54, 56, 57, 58, 59, 60, 93, 96, 97, 99, 100, and 101; and (d) an HCVD that is at least 90%, 95%, or 98% identical to SEQ ID NO: 234, where the HCVD includes a histidine at one or more positions in SEQ ID NO: 234; and/or an LCVD that is at least 90%, 95%, or 98% identical to SEQ ID NO: 235, where the LCVD includes a histidine at one or more positions in SEQ ID NO: 235. In each of the foregoing embodiments, an alanine may be substituted where a histidine was already present.

In some embodiments, the first ABD includes an HCVD that is one of (a) to (d): (a) an HCVD of SEQ ID NO: 4, one of the sequences set forth in SEQ ID NOs: 12-52, or one of the sequences set forth in SEQ ID NOs: 313-350; (b) an HCVD of SEQ ID NO: 80, one of the sequences set forth in SEQ ID NOs: 88-128, one of the sequences set forth in SEQ ID NOs: 397-406, or one of the sequences set forth in SEQ ID NOs: 397-406; (c) an HCVD of SEQ ID NO: 156, one of the sequences set forth in SEQ ID NOs: 164-202, or one of the sequences set forth in SEQ ID NOs: 427-457; and (d) an HCVD of SEQ ID NO: 234, or one of the sequences set forth in SEQ ID NOs: 242-280; and/or the first ABD includes an LCVD of one of (a) to (d): (a) an LCVD of SEQ ID NO: 5, one of the sequences set forth in SEQ ID NOs: 53-79, or one of the sequences set forth in SEQ ID NOs: 351-396; (b) an LCVD of SEQ ID NO: 81, one of the sequences set forth in SEQ ID NOs: 129-155, or one of the sequences set forth in SEQ ID NOs: 407-426; (c) an LCVD of SEQ ID NO: 157, one of the sequences set forth in SEQ ID NOs: 203-233, or one of the sequences set forth in SEQ ID NOs: 458-492; and (d) an LCVD of SEQ ID NO: 235, or one of the sequences set forth in SEQ ID NOs: 281-312. In each of the foregoing embodiments, an alanine may be substituted where a histidine was already present.

In some embodiments, the first ABD includes one of (a) to (d): (a) an HCVD of SEQ ID NO: 4, one of the sequences set forth in SEQ ID NOs: 12-52, or one of the sequences set forth in SEQ ID NOs: 313-350, and/or an LCVD of SEQ ID NO: 5, one of the sequences set forth in SEQ ID NOs: 53-79, or one of the sequences set forth in SEQ ID NOs: 351-396, where the first ABD does not include (i) an HCVD of SEQ ID NO: 4 and an LCVD of SEQ ID NO: 5; (ii) an HCVD of SEQ ID NO: 4 and LCVD that is not one of SEQ ID NOs: 57, 58, 59, 60, 61, 62, 64, 65, 66, 67, 71, 74, 75, 76, 77, 78, 79, 351, 352, 353, 354, 355, 359, 360, 361, 362, 365, 366, 367, 368, 370, 371, 372, 373, 376, 377, 383, 386, 387, 388, 389, 392, 393, 394, 395, and 396; or (iii) an HCVD that is not one of SEQ ID NOs: 15, 18, 19, 22, 24, 25, 32, 33, 34, 35, 36, 37, 38, 41, 43, 46, 47, 50, 318, 320, 322, 323, 324, 325, 328, 332, 333, 334, 336, 337, 343, 344, 345, 347, and 348 and an LCVD of SEQ ID NO: 5; (b) an HCVD of SEQ ID NO: 80, one of the sequences set forth in SEQ ID NOs: 88-128, one of the sequences set forth in SEQ ID NOs: 397-406, or one of the sequences set forth in SEQ ID NOs: 397-406; and/or an LCVD of SEQ ID NO: 81, one of the sequences set forth in SEQ ID NOs: 129-155, or one of the sequences set forth in SEQ ID NOs: 407-426, where the first ABD does not include (i) an HCVD of SEQ ID NO: 80 and an LCVD of SEQ ID NO: 81; (ii) an HCVD of SEQ ID NO: 80 and an LCVD that is not one of SEQ ID NOs: 131, 133, 134, 135, 136, 138, 139, 140, 141, 142, 143, 144, 147, 151, 152, 153, 411, 412, 417, 418, and 423; or (iii) an HCVD that is not one of SEQ ID NOs: 91, 101, 103, 112, 113, 116, 119, 122, 123, 125, and 126 and an LCVD of SEQ ID NO: 81; (c) an HCVD of SEQ ID NO: 156, one of the sequences set forth in SEQ ID NOs: 164-202, or one of the sequences set forth in SEQ ID NOs: 427-457; and/or an LCVD of SEQ ID NO: 157, one of the sequences set forth in SEQ ID NOs: 203-233, or one of the sequences set forth in SEQ ID NOs: 458-492, where the first ABD does not include (i) an HCVD of SEQ ID NO: 156 and an LCVD of SEQ ID NO: 157; (ii) an HCVD of SEQ ID NO: 156 and an LCVD that is not one of the sequences set forth in SEQ ID NOs: 204, 211, 213, 215, 218, 220, 221, 222, 223, 224, 225, 228, 229, 231, 232, 233, 459, 460, 462, 464, 465, 466, 467, 468, 469, 470, 473, 474, 485, 488, 489, and 491; or (iii) an HCVD that is not one of the sequences set forth in SEQ ID NOs: 164, 167, 168, 171, 172, 175, 178, 180, 181, 183, 196, 197, 200, 201, 427, 428, 429, 430, 431, 433, 434, 435, 436, 437, 438, 439, 440, 441, 443, 444, 445, 446, 447, 449, 451, 454, 456, and 457 and an LCVD of SEQ ID NO: 157; and (d) an HCVD of SEQ ID NO: 234, or one of the sequences set forth in SEQ ID NOs: 242-280; and/or an LCVD of SEQ ID NO: 235 or one of the sequences set forth in SEQ LD NOs: 281-312, where the first ABD does not include an HCVD of SEQ ID NO: 234 and an LCVD of SEQ ID NO: 235.

In some embodiments, the ABPC is degraded in the target mammalian cell following internalization of the ABPC by the target mammalian cell. In some embodiments, the ABPC includes a conjugated toxin, radioisotope, drug, or small molecule.

In some embodiments, a composition including the ABPC provides for an increase in toxin liberation in the target mammalian cell as compared to a composition including the same amount of a control ABPC. In some embodiments, a composition including the ABPC provides for at least a 20% or 50% increase in toxin liberation in the target mammalian cell as compared to a composition including the same amount of a control ABPC. In some embodiments, a composition including the ABPC provides for at least a 2- or 5-fold increase in toxin liberation in the target mammalian cell as compared to a composition including the same amount of a control ABPC.

In some embodiments, a composition including the ABPC provides for an increase in target mammalian cell killing as compared to a composition including the same amount of a control ABPC. In some embodiments, a composition including the ABPC provides for at least a 20% or 50% increase in target mammalian cell killing as compared to a composition including the same amount of a control ABPC. In some embodiments, a composition including the ABPC provides for at least a 2- or 5-fold increase in target mammalian cell killing as compared to a composition including the same amount of a control ABPC.

In some embodiments, a composition including the ABPC provides for an increase in endolysosomal delivery in the target mammalian cell as compared to a composition including the same amount of a control ABPC. In some embodiments, the ABPC provides for at least a 20% increase in endolysosomal delivery in the target mammalian cell as compared to a composition including the same amount of a control ABPC. In some embodiments, a composition including the ABPC provides for at least a 50% increase in endolysosomal delivery in the target mammalian cell as compared to a composition including the same amount of a control ABPC. In some embodiments, a composition including the ABPC provides for at least a 2-fold increase in endolysosomal delivery in the target mammalian cell as compared to a composition including the same amount of a control ABPC. In some embodiments, a composition including the ABPC provides for at least a 5-fold increase in endolysosomal delivery in the target mammalian cell as compared to a composition including the same amount of a control ABPC.

In some embodiments, a composition including the ABPC results in a less of a reduction in the level of TROP2 presented on the surface of the target mammalian cell as compared to a composition including the same amount of a control ABPC. In some embodiments, a composition including the ABPC does not result in a detectable reduction in the level of the TROP2 presented on the surface of the target mammalian cell.

Also provided herein are antigen-binding protein constructs (ABPCs) including: a first ABD that is capable of specifically binding TROP2 or an epitope of TROP2 presented on the surface of a target mammalian cell; and a conjugated toxin, radioisotope, drug, or small molecule, where: (a) the dissociation rate of the first ABD at a pH of about 4.0 to about 6.5 is faster than the dissociation rate at a pH of about 7.0 to about 8.0; or the dissociation constant (K_{D}) of the first ABD at a pH of about 4.0 to about 6.5 is greater than the K_{D} at a pH of about 7.0 to about 8.0; and (b) the composition provides for one or more of: an increase in toxin liberation in the target mammalian cell as compared to a composition including the same amount of a control ABPC; an increase in target mammalian cell killing as compared to a composition including the same amount of a control ABPC; and an increase in endolysosomal delivery in the target mammalian cell as compared to a composition including the same amount of a control ABPC.

In some embodiments, the first ABD includes one of (a) to (d): (a) an HCVD of sacituzumab; (b) an HCVD of datopotamab; (c) an HCVD of hu7E6SVG; and (d) an HCVD of A1,2X4, each or any with one or more amino acids substituted with a histidine. In some embodiments, the first ABD includes an LCVD of one of (a) to (d): (a) an LCVD of sacituzumab; (b) an LCVD of datopotamab; (c) an LCVD of hu7E6SVG; and (d) an LCVD of A1,2X4 each or any with one or more amino acids substituted with a histidine. In some embodiments, the first ABD includes one of (a) to (d): (a) an HCVD of sacituzumab and an LCVD of sacituzumab; (b) an HCVD of datopotamab and an LCVD of datopotamab; (c) an HCVD of hu7E6SVG and an LCVD of hu7E6SVG; and (d) an HCVD of A1,2X4 and an LCVD of A1,2X4, each or any of the domains with one or more amino acids substituted with a histidine. In each of the foregoing embodiments, an alanine may be substituted where a histidine was already present.

In some embodiments, the HCVD includes one of (a) to (d): (a) an HCVD of sacituzumab including SEQ ID NO: 4; (b) an HCVD of datopotamab including SEQ ID NO: 80; (c) an HCVD of hu7E6SVG including SEQ ID NO: 156; and (d) an HCVD of A1,2X4 including SEQ ID NO: 234. In some embodiments, the LCVD includes one of (a) to (d): (a) an LCVD of sacituzumab including SEQ ID NO: 5; (b) an LCVD of datopotamab including SEQ ID NO: 81; (c) an LCVD of hu7E6SVG including SEQ ID NO: 157; and (d) an LCVD of A1,2X4 including SEQ ID NO: 235. In each of the foregoing embodiments, an alanine may be substituted where a histidine was already present.

In some embodiments, the first ABD includes an HCVD that is one of (a) to (d): (a) an HCVD including a CDR1, a CDR2, and a CDR3 of SEQ ID NOs: 6, 7, and 8, respectively, with collectively a total of one or more amino acid positions in SEQ ID NOs: 6, 7, and 8 substituted with a histidine; (b) an HCVD including a CDR1, a CDR2, and a CDR3 of SEQ ID NOs: 82, 83, and 84, respectively, with collectively a total of one or more amino acid positions in SEQ ID NOs: 82, 83, and 84 substituted with a histidine; (c) an HCVD including a CDR1, a CDR2, and a CDR3 of SEQ ID NOs: 158, 159, and 160, respectively, with collectively a total of one or more amino acid positions in SEQ ID NOs: 158, 159, and 160 substituted with a histidine; and (d) an HCVD including a CDR1, a CDR2, and a CDR3 of SEQ ID NOs: 236, 237, and 238, respectively, with collectively a total of one or more amino acid positions in SEQ ID NOs: 236, 237, and 238 substituted with a histidine. In each of the foregoing embodiments, an alanine may be substituted where a histidine was already present.

In some embodiments, the first ABD includes an LCVD of one of (a) to (d): (a) an LCVD including a CDR1, a CDR2, and a CDR3 of SEQ ID NOs: 9, 10, and 11, respectively, with collectively a total of one or more amino acid positions in SEQ ID NOs: 9, 10, and 11 substituted with a histidine; (b) an LCVD including a CDR1, a CDR2, and a CDR3 of SEQ ID NOs: 85, 86, and 87, respectively, with collectively a total of one or more amino acid positions in SEQ ID NOs: 85, 86, and 87 substituted with a histidine; (c) an LCVD including a CDR1, a CDR2, and a CDR3 of SEQ ID NOs: 161, 162, and 163, respectively, with collectively a total of one or more amino acid positions in SEQ ID NOs: 161, 162, and 163 substituted with a histidine; and (d) an LCVD including a CDR1, a CDR2, and a CDR3 of SEQ ID NOs: 239, 240, and 241, respectively, with collectively a total of one or more amino acid positions in SEQ ID NOs: 239, 240, and 241 substituted with a histidine. In each of the foregoing embodiments, an alanine may be substituted where a histidine was already present.

In some embodiments, the first ABD includes one of (a) to (d): (a) an HCVD including a CDR1, a CDR2, and a CDR3 of SEQ ID NOs: 6, 7, and 8, respectively, with collectively a total of one or more amino acid positions in SEQ ID NOs: 6, 7, and 8 substituted with a histidine; and/or an LCVD including a CDR1, a CDR2, and a CDR3 of SEQ ID NOs: 9, 10, and 11, respectively, with collectively a total of one or more amino acid positions in SEQ ID NOs: 9, 10, and 11 substituted with a histidine; (b) an HCVD including a CDR1, a CDR2, and a CDR3 of SEQ ID NOs: 82, 83, and 84, respectively, with collectively a total of one or more amino acid positions in SEQ ID NOs: 82, 83, and 84 substituted with a histidine; and/or an LCVD including a CDR1, a CDR2, and a CDR3 of SEQ ID NOs: 85, 86, and 87, respectively, with collectively a total of one or more amino acid positions in SEQ ID NOs: 85, 86, and 87 substituted with a histidine; (c) an HCVD including a CDR1, a CDR2, and a CDR3 of SEQ ID NOs: 158, 159, and 160, respectively, with collectively a total of one or more amino acid positions in SEQ ID NOs: 158, 159, and 160 substituted with a histidine; and/or an LCVD including a CDR1, a CDR2, and a CDR3 of SEQ ID NOs: 161, 162, and 163, respectively, with collectively a total of one or more amino acid positions in SEQ ID NOs: 161, 162, and 163 substituted with a histidine; and (d) an HCVD including a CDR1, a CDR2, and a CDR3 of SEQ ID NOs: 236, 237, and 238, respectively, with collectively a total of one or more amino acid positions in SEQ ID NOs: 236, 237, and 238 substituted with a histidine; and/or an LCVD including a CDR1, a CDR2, and a CDR3 of SEQ ID NOs: 239, 240, and 241, respectively, with collectively a total of one or more amino acid positions in SEQ ID NOs: 239, 240, and 241 substituted with a histidine. In each of the foregoing embodiments, an alanine may be substituted where a histidine was already present.

In some embodiments, the first ABD includes an HCVD that is one of (a) to (d): (a) an HCVD that is at least 90%, 95%, or 98% identical to SEQ ID NO: 4, where the HCVD includes a histidine at one or more positions in SEQ ID NO: 4 selected from: 29, 32, 33, 50, 52, 53, 60, 61, 62, 63, 64, 65, 66, 99, 101, 104, 105, and 108; (b) an HCVD that is at least 90%, 95%, or 98% identical to SEQ ID NO: 80, where the HCVD includes a histidine at one or more positions in SEQ ID NO: 80 selected from: 29, 53, 55, 64, 65, 98, 101, 104, 105, 107, and 108; (c) an HCVD that is at least 90%, 95%, or 98% identical to SEQ ID NO: 156, where the HCVD includes a histidine at one or more positions in SEQ ID NO: 156 selected from: 26, 29, 30, 33, 34, 51, 54, 56, 57, 59, 102, 103, and 106, and (d) an HCVD that is at least 90%, 95%, or 98% identical to SEQ ID NO: 234, where the HCVD includes a histidine at one or more positions in SEQ ID NO: 234. In each of the foregoing embodiments, an alanine may be substituted where a histidine was already present.

In some embodiments, the first ABD includes an LCVD of one of (a) to (d): (a) an LCVD that is at least 90%, 95%, or 98% identical to SEQ ID NO: 5, where the LCVD includes a histidine at one or more positions in SEQ ID NO: 5 selected from: 28, 29, 30, 31, 32, 33, 50, 51, 52, 53, 89, 92, 93, 94, 95, 96, and 97; (b) an LCVD that is at least 90%, 95%, or 98% identical to SEQ ID NO: 81, where the LCVD includes a histidine at one or more positions in SEQ ID NO: 81 selected from: 26, 28, 29, 30, 31, 33, 34, 50, 51, 52, 53, 54, 89, 93, 94, and 95; (c) an LCVD that is at least 90%, 95%, or 98% identical to SEQ ID NO: 157, where the LCVD includes a histidine at one or more positions in SEQ ID NO: 157 selected from 25, 32, 34, 36, 54, 56, 57, 58, 59, 60, 93, 96, 97, 99, 100, and 101; and (d) an LCVD that is at least 90%, 95%, or 98% identical to SEQ ID NO: 235, where the LCVD includes a histidine at one or more positions in SEQ ID NO: 235. In each of the foregoing embodiments, an alanine may be substituted where a histidine was already present.

In some embodiments, the first ABD includes an HCVD that is one of (a) to (d): (a) an HCVD that is at least 90%, 95%, or 98% identical to SEQ ID NO: 4, where the HCVD includes a histidine at two or more positions in SEQ ID NO: 4 selected from: 29, 32, 33, 50, 52, 53, 60, 61, 62, 63, 64, 65, 66, 99, 101, 104, 105, and 108; (b) an HCVD that is at least 90%, 95%, or 98% identical to SEQ ID NO: 80, where the HCVD includes a histidine at two or more positions in SEQ ID NO: 80 selected from 29, 53, 55, 64, 65, 98, 101, 104, 105, 107, and 108; (c) an HCVD that is at least 90%, 95%, or 98% identical to SEQ LD NO: 156, where the HCVD includes a histidine at two or more positions in SEQ ID NO: 156 selected from 26, 29, 30, 33, 34, 51, 54, 56, 57, 59, 102, 103, and 106; and (d) an HCVD that is at least 90%, 95%, or 98% identical to SEQ ID NO: 234, where the HCVD includes a histidine at two or more positions in SEQ ID NO: 234. In each of the foregoing embodiments, an alanine may be substituted where a histidine was already present.

In some embodiments, the first ABD includes an LCVD of one of (a) to (d): (a) an LCVD that is at least 90%, 95%, or 98% identical to SEQ ID NO: 5, where the LCVD includes a histidine at two or more positions in SEQ ID NO: 5 selected from: 28, 29, 30, 31, 32, 33, 50, 51, 52, 53, 89, 92, 93, 94, 95, 96, and 97; (b) an LCVD that is at least 90%, 95%, or 98% identical to SEQ ID NO: 81, where the LCVD includes a histidine at two or more positions in SEQ ID NO: 81 from: 26, 28, 29, 30, 31, 33, 34, 50, 51, 52, 53, 54, 89, 93, 94, and 95; (c) an LCVD that is at least 90%, 95%, or 98% identical to SEQ ID NO: 157, where the LCVD includes a histidine at two or more positions in SEQ ID NO: 157 selected from: 25, 32, 34, 36, 54, 56, 57, 58, 59, 60, 93, 96, 97, 99, 100, and 101; and (d) an LCVD that is at least 900%, 95%, or 98% identical to SEQ ID NO: 235, where the LCVD includes a histidine at two or more positions in SEQ ID NO: 235. In each of the foregoing embodiments, an alanine may be substituted where a histidine was already present.

In some embodiments, the first ABD includes one of (a) to (d): (a) an HCVD that is at least 90%, 95%, or 98% identical to SEQ ID NO: 4, where the HCVD includes a histidine at one or more positions in SEQ ID NO: 4 selected from: 29, 32, 33, 50, 52, 53, 60, 61, 62, 63, 64, 65, 66, 99, 101, 104, 105, and 108; and/or an LCVD that is at least 90%, 95%, or 98% identical to SEQ ID NO: 5, where the LCVD includes a histidine at one or more positions in SEQ ID NO: 5 selected from: 28, 29, 30, 31, 32, 33, 50, 51, 52, 53, 89, 92, 93, 94, 95, 96, and 97; (b) an HCVD that is at least 90%, 95%, or 98% identical to SEQ ID NO: 80, where the HCVD includes a histidine at one or more positions in SEQ ID NO: 80 selected from: 29, 53, 55, 64, 65, 98, 101, 104, 105, 107, and 108; and/or an LCVD that is at least 90%, 95%, or 98% identical to SEQ ID NO: 81, where the LCVD includes a histidine at one or more positions in SEQ ID NO: 81 selected from: 26, 28, 29, 30, 31, 33, 34, 50, 51, 52, 53, 54, 89, 93, 94, and 95; (c) an HCVD that is at least 90%, 95%, or 98% identical to SEQ ID NO: 156, where the HCVD includes a histidine at one or more positions in SEQ ID NO: 156 selected from: 26, 29, 30, 33, 34, 51, 54, 56, 57, 59, 102, 103, and 106; and/or an LCVD that is at least 90%, 95%, or 98% identical to SEQ ID NO: 157, where the LCVD includes a histidine at one or more positions in SEQ ID NO: 157 selected from 25, 32, 34, 36, 54, 56, 57, 58, 59, 60, 93, 96, 97, 99, 100, and 101; and (d) an HCVD that is at least 90%, 95%, or 98% identical to SEQ ID NO: 234, where the HCVD includes a histidine at one or more positions in SEQ ID NO: 234; and/or an LCVD that is at least 90%, 95%, or 98% identical to SEQ ID NO: 235, where the LCVD includes a histidine at one or more positions in SEQ ID NO: 235. In each of the foregoing embodiments, an alanine may be substituted where a histidine was already present.

In some embodiments, the first ABD includes one of (a) to (d): (a) an HCVD of SEQ ID NO: 4, one of the sequences set forth in SEQ ID NOs: 12-52, or one of the sequences set forth in SEQ ID NOs: 313-350; (b) an HCVD of SEQ ID NO: 80, one of the sequences set forth in SEQ ID NOs: 88-128, one of the sequences set forth in SEQ ID NOs: 397-406, or one of the sequences set forth in SEQ ID NOs: 397-406; (c) an HCVD of SEQ ID NO: 156, one of the sequences set forth in SEQ ID NOs: 164-202, or one of the sequences set forth in SEQ ID NOs: 427-457; and (d) an HCVD of SEQ ID NO: 234, or one of the sequences set forth in SEQ ID NOs: 242-280. In some embodiments, the first ABD includes one of (a) to (d): (a) an LCVD of SEQ ID NO: 5, one of the sequences set forth in SEQ ID NOs: 53-79, or one of the sequences set forth in SEQ ID NOs: 351-396; (b) an LCVD of SEQ ID NO: 81, one of the sequences set forth in SEQ ID NOs: 129-155, or one of the sequences set forth in SEQ ID NOs: 407-426; (c) an LCVD of SEQ ID NO: 157, one of the sequences set forth in SEQ ID NOs: 203-233, or one of the sequences set forth in SEQ ID NOs: 458492; and (d) an LCVD of SEQ ID NO: 235, or one of the sequences set forth in SEQ ID NOs: 281-312.

In some embodiments, the first ABD includes one of (a) to (d): (a) an HCVD of SEQ ID NO: 4, one of the sequences set forth in SEQ ID NOs: 12-52, or one of the sequences set forth in SEQ ID NOs: 313-350, and/or an LCVD of SEQ ID NO: 5, one of the sequences set forth in SEQ ID NOs: 53-79, or one of the sequences set forth in SEQ ID NOs: 351-396, where the first ABD does not include (i) an HCVD of SEQ ID NO: 4 and an LCVD of SEQ ID NO: 5; (ii) an HCVD of SEQ ID NO: 4 and an LCVD that is not one of SEQ ID NOs: 57, 58, 59, 61, 62, 64, 65, 66, 67, 71, 74, 75, 76, 77, 78, 79, 351, 352, 353, 354, 355, 359, 360, 361, 362, 365, 366, 367, 368, 370, 371, 372, 373, 376, 377, 383, 386, 387, 388, 389, 392, 393, 394, 395, and 396; or (iii) an HCVD that is not one of SEQ ID NOs: 15, 18, 19, 22, 24, 25, 32, 33, 34, 35, 36, 37, 38, 41, 43, 46, 47, 50, 318, 320, 322, 323, 324, 325, 328, 332, 333, 334, 336, 337, 343, 344, 345, 347, and 348 and an LCVD of SEQ ID NO: 5; (b) an HCVD of SEQ ID NO: 80, one of the sequences set forth in SEQ ID NOs: 88-128, one of the sequences set forth in SEQ ID NOs: 397-406, or one of the sequences set forth in SEQ ID NOs: 397-406; and/or an LCVD of SEQ ID NO: 81, one of the sequences set forth in SEQ ID NOs: 129-155, or one of the sequences set forth in SEQ ID NOs: 407-426, where the first ABD does not include (i) an HCVD of SEQ ID NO: 80 and an LCVD of SEQ ID NO: 81; (ii) an HCVD of SEQ ID NO: 80 and an LCVD that is not one of SEQ ID NOs: 131, 133, 134, 135, 136, 138, 139, 140, 141, 142, 143, 144, 147, 151, 152, 153,411, 412,417, 418, and 423; or (iii) an HCVD that is not one of SEQ ID NOs: 91, 101, 103, 112, 113, 116, 119, 122, 123, 125, and 126 and an LCVD of SEQ ID NO: 81; (c) an HCVD of SEQ ID NO: 156, one of the sequences set forth in SEQ ID NOs: 164-202, or one of the sequences set forth in SEQ ID NOs: 427-457; and/or an LCVD of SEQ ID NO: 157, one of the sequences set forth in SEQ ID NOs: 203-233, or one of the sequences set forth in SEQ ID NOs: 458-492, where the first ABD does not include (i) an HCVD of SEQ ID NO: 156 and an LCVD of SEQ ID NO: 157; (ii) an HCVD of SEQ ID NO: 156 and an LCVD that is not one of the sequences set forth in SEQ ID NOs: 204, 211, 213, 215, 218, 220, 221, 222, 223, 224, 225, 228, 229, 231, 232, 233, 459, 460, 462, 464, 465, 466, 467, 468, 469, 470, 473, 474, 485, 488, 489, and 491; or (iii) an HCVD that is not one of the sequences set forth in SEQ ID NOs: 164, 167, 168, 171, 172, 175, 178, 180, 181, 183, 196, 197, 200, 201, 427, 428, 429, 430, 431, 433, 434, 435, 436, 437, 438, 439, 440, 441, 443, 444, 445, 446, 447, 449, 451, 454, 456, and 457 and an LCVD of SEQ ID NO: 157; and (d) an HCVD of SEQ ID NO: 234 or one of the sequences set forth in SEQ ID NOs: 242-280; and/or an LCVD of SEQ ID NO: 235 or one of the sequences set forth in SEQ ID NOs: 281-312, where the first ABD does not include an HCVD of SEQ ID NO: 234.

In some embodiments, a composition including the ABPC provides for an increase in toxin liberation in the target mammalian cell as compared to a composition including the same amount of a control ABPC. In some embodiments, a composition including the ABPC provides for at least a 20% increase in toxin liberation in the target mammalian cell as compared to a composition including the same amount of a control ABPC. In some embodiments, a composition including the ABPC provides for at least a 50% increase in toxin liberation in the target mammalian cell as compared to a composition including the same amount of a control ABPC. In some embodiments, a composition including the ABPC provides for at least a 2-fold increase in toxin liberation in the target mammalian cell as compared to a composition including the same amount of a control ABPC. In some embodiments, a composition including the ABPC provides for at least a 5-fold increase in toxin liberation in the target mammalian cell as compared to a composition including the same amount of a control ABPC.

In some embodiments, a composition including the ABPC provides for an increase in target mammalian cell killing as compared to a composition including the same amount of a control ABPC In some embodiments, a composition including the ABPC provides for at least a 20% or 50% increase in target mammalian cell killing as compared to a composition including the same amount of a control ABPC. In some embodiments, a composition including the ABPC provides for at least a 2- or 5-fold increase in target mammalian cell killing as compared to a composition including the same amount of a control ABPC.

In some embodiments, a composition including the ABPC provides for an increase in endolysosomal delivery in the target mammalian cell as compared to a composition including the same amount of a control ABPC. In some embodiments, a composition including the ABPC provides for at least a 20% or 50% increase in endolysosomal delivery in the target mammalian cell as compared to a composition including the same amount of a control ABPC. In some embodiments, a composition including the ABPC provides for at least a 2- or 5-fold increase in endolysosomal delivery in the target mammalian cell as compared to a composition including the same amount of a control ABPC.

In some embodiments, a composition including the ABPC results in a less of a reduction in the level of TROP2 presented on the surface of the target mammalian cell as compared to a composition including the same amount of a control ABPC. In some embodiments, a composition including the ABPC does not result in a detectable reduction in the level of the TROP2 presented on the surface of the target mammalian cell. In some embodiments, the target mammalian cell is a cancer cell.

In some embodiments, the dissociation rate of the ABD at a pH of ~4.0 to ~6.5 is at least 10% faster than the dissociation rate of the ABD at a pH of ~7.0 to ~8.0. In some embodiments, the dissociation rate of the ABD at a pH of ~4.0 to ~6.5 is at least 3-fold faster than the dissociation rate of the ABD at a pH of ~7.0 to ~8.0. In some embodiments, the dissociation rate of the ABD at a pH of ~4.0 to ~6.5 is at least 10-fold faster than the dissociation rate of the ABD at a pH of ~7.0 to ~8.0. In some embodiments, the K_{D} of the ABD at a pH of ~4.0 to ~6.5 is at least 10% greater than the K_{D} of the ABD at a pH of ~7.0 to ~8.0. In some embodiments, the K_{D} of the ABD at a pH of ~4.0 to ~6.5 is at least 3-fold greater than the K_{D} of the ABD at a pH of ~7.0 to ~8.0. In some embodiments, the K_{D} of the ABD at a pH of ~4.0 to ~6.5 is at least 10-fold greater than the KU of the ABD at a pH of ~7.0 to ~8.0.

In some embodiments, the ABPC is cytotoxic or cytostatic to the target mammalian cell.

In some embodiments, the ABPC is cross-reactive with a non-human primate TROP2 and human TROP2. In some embodiments, the ABPC is cross-reactive with a non-human primate TROP2, a human TROP2, and one or both of rat TROP2 and a mouse TROP2. In some embodiments, the ABPC is cross-reactive with a non-human primate TROP2, a human TROP2, a rat TROP2, and a mouse TROP2. In some embodiments, the ABD binds to an epitope of TROP2 that is present on the surface of cells from an Old World Monkey.

In some embodiments, the ABPC includes a single polypeptide. In some embodiments, the ABD is selected from a VH domain, a VHH domain, a VNAR domain, and a scFv. In some embodiments, the ABPC is a BiTe, a (scFv)₂, a nanobody, a nanobody-HSA, a DART, a TandAb, a scDiabody, a scDiabody-CH3, scFv-CH-CL-scFv, a HSAbody, scDiabody-HSA, or a tandem-scFv

In some embodiments, the ABPC includes two or more polypeptides. In some embodiments, the ABPC is selected from the group of an antibody, a VHH-scAb, a VHH-Fab, a Dual scFab, a F(ab′)2, a diabody, a crossMab, a DAF (two-in-one), a DAF (four-in-one), a DutaMab, a DT-IgG, a knobs-in-holes common light chain, a knobs-in-holes assembly, a charge pair, a Fab-arm exchange, a SEEDbody, a LUZ-Y, a Fcab, a X-body, an orthogonal Fab, a DVD-IgG, a IgG(H)-scFv, a scFv-(H)IgG, IgG(L)-scFv, scFv-(L)IgG, IgG(L,H)-Fv, IgG(H)-V, V(H)-IgG, IgG(L)-V, V(L)-IgG, KIH IgG-scFab, 2scFv-IgG, IgG-2scFv, scFv4-Ig, Zybody, DVI-IgG, Diabody-CH3, a triple body, a miniantibody, a minibody, a TriBi minibody, scFv-CH3 KIH, Fab-scFv, a F(ab′)2-scFv2, a scFv-KIH, a Fab-scFv-Fc, a tetravalent HCAb, a scDiabody-Fc, a Diabody-Fc, a tandem scFv-Fc, a VHH-Fc, a tandem VHH-Fc, a VHH-Fc KiH, a Fab-VHH-Fc, an Intrabody, a dock and lock, an ImmTAC, an IgG-IgG conjugate, a Cov-X-Body, a scFv1-PEG-scFv2, an Adnectin, a DARPin, a fibronectin, a DEP conjugate, a PROTAB, and a PROTAC.

In some embodiments, at least one polypeptide of the ABPC is conjugated to the toxin, the radioisotope, the drug, or the small molecule via a cleavable linker. In some embodiments, at least one polypeptide of the ABPC is conjugated to the toxin, the radioisotope, the drug, or the small molecule via a non-cleavable linker.

In some embodiments, the half-life of the ABPC in vivo is decreased as compared to the half-life of a control ABPC in vivo. In some embodiments, the ABPC in vivo is decreased about 5% to 95% as compared to the half-life of a control ABPC in vivo. In some embodiments, the half-life of the ABPC in vivo is decreased about 10% to 95% as compared to the half-life of a control ABPC in vivo. In some embodiments, the half-life of the ABPC in vivo is decreased about 30% to 95% as compared to the half-life of a control ABPC in vivo. In some embodiments, the half-life of the ABPC in vivo is decreased about 50% to 95% as compared to the half-life of a control ABPC in vivo. In some embodiments, the half-life of the ABPC in vivo is decreased about 70% to 95% as compared to the half-life of a control ABPC in vivo.

In some embodiments, the control ABPC is capable of specifically binding to TROP2 or an epitope of TROP2 presented on the surface of a target mammalian cell, where: (a) the control ABPC includes a first ABD; (b) the dissociation rate of the first ABD of the control ABPC at a pH of ~4.0 to ~6.5 is no more than 3-fold faster than the dissociation rate at a pH of ~7.0 to ~8.0; and (c) the dissociation constant (K_{D}) of the first ABD of the control ABPC at a pH of ~4.0 to ~6.5 is no more than 3-fold greater than the K_{D} at a pH of ~7.0 to ~8.0.

In some embodiments, the control ABPC is capable of specifically binding to TROP2 or an epitope of TROP2 presented on the surface of a target mammalian cell, where: (a) the control ABPC includes a first ABD; (b) the dissociation rate of the first ABD of the control ABPC at a pH of ~4.0 to ~6.5 is no more than 2-fold faster than the dissociation rate at a pH of ~7.0 to ~8.0; and (c) the dissociation constant (K_{D}) of the first ABD of the ABPC at a pH of ~4.0 to ~6.5 is no more than 2-fold greater than the K_{D} at a pH of ~7.0 to ~8.0.

In some embodiments, the control ABPC is capable of specifically binding to TROP2 or an epitope of TROP2 presented on the surface of a target mammalian cell, where: (a) the control ABPC includes a first ABD; (b) the dissociation rate of the first ABD of the control ABPC at a pH of about 4.0 to about 6.5 is no more than 1-fold faster than the dissociation rate at a pH of about 7.0 to about 8.0; and (c) the dissociation constant (K_{D}) of the first ABD of the control ABPC at a pH of about 4.0 to about 6.5 is no more than 1-fold greater than the K) at a pH of about 7.0 to about 8.0.

In some embodiments, the control ABPC is sacituzumab, datopotamab, hu7E6SVG, or A1,2X4.

In some embodiments, the ABPC includes a second ABD.

Also provided herein are kits including at least one dose of any ABPC described herein.

Also provided herein are methods of treating a cancer characterized by having a population of cancer cells that have TROP2 or an epitope of TROP2 presented on their surface, the method including: administering a therapeutically effective amount of any of the pharmaceutical compositions described herein or any of the ABPCs described herein to a subject identified as having a cancer characterized by having the population of cancer cells.

Also provided herein are methods of reducing the volume of a tumor in a subject, where the tumor is characterized by having a population of cancer cells that have TROP2 or an epitope of TROP2 presented on their surface, the method including: administering a therapeutically effective amount of any of the pharmaceutical compositions described herein or any of the ABPCs described herein to a subject identified as having a cancer characterized by having the population of cancer cells.

Also provided herein are methods of inducing cell death in a cancer cell in a subject, where the cancer cell has TROP2 or an epitope of TROP2 presented on its surface, where the method includes: administering a therapeutically effective amount of any of the pharmaceutical compositions described herein or any of the ABPCs described herein to a subject identified as having a cancer characterized by having a population of the cancer cells.

In some embodiments, the cancer is a primary tumor. In some embodiments, the cancer is a metastasis.

In some embodiments, the cancer is a non-T-cell-infiltrating tumor. In some embodiments, the cancer is a T-cell infiltrating tumor.

Also provided herein are methods of decreasing the risk of developing a metastasis or decreasing the risk of developing an additional metastasis in a subject having a cancer, where the cancer is characterized by having a population of cancer cells that have TROP2 or an epitope of TROP2 presented on their surface the method including: administering a therapeutically effective amount of any one of the pharmaceutical compositions described herein or any of the ABPCs described herein to a subject identified as having a cancer characterized by having the population of cancer cells.

In some embodiments, the cancer is a non-T-cell-infiltrating tumor. In some embodiments, the cancer is a T-cell infiltrating tumor.

As used herein, the term “antigen-binding protein construct” is (i) a single polypeptide that includes at least one ABD or (ii) a complex of two or more polypeptides (e.g., the same or different polypeptides) that together form at least one ABD. Non-limiting examples and aspects of antigen-binding protein constructs are described herein. Additional examples and aspects of antigen-binding protein constructs are known in the art.

A “multi-specific ABPC” is an ABPC that includes two or more different ABDs that collectively specifically bind two or more different epitopes. The two or more different epitopes may be epitopes on the same antigen (e.g., a single polypeptide present on the surface of a cell) or on different antigens (e.g., different proteins present on the surface of the same cell or present on the surface of different cells). In some aspects, the antigen is present on the surface of the cell. In some aspects, a multi-specific ABPC binds two different epitopes (i.e., a “bispecific ABPC”). In some aspects, a multi-specific ABPC binds three different epitopes (i.e., a “trispecific ABPC”). In some aspects, a multi-specific ABPC binds four different epitopes (i.e., a “quadspecific ABPC”). In some aspects, a multi-specific ABPC binds five different epitopes (i.e., a “quintspecific ABPC”). Each binding specificity may be present in any suitable valency. Non-limiting examples of multi-specific ABPC s are described herein.

An “Antigen-Binding Protein” or “ABD” is one or more protein domain(s) (e.g., formed from amino acids from a single polypeptide or formed from amino acids from two or more polypeptides (e.g., the same or different polypeptides) that is capable of specifically binding to one or more different antigen(s). In some examples, an ABD can bind to an antigen or epitope with specificity and affinity similar to that of naturally-occurring antibodies. In some embodiments, the ABD can be an antibody or a fragment thereof. In some embodiments, an ABD can include an alternative scaffold. Non-limiting examples of ABDs are described herein. Additional examples of ABDs are known in the art. In some examples, an ABD can bind to a single antigen.

The term “antibody” is used herein in its broadest sense and includes certain types of immunoglobulin molecules that include one or more ABDs that specifically bind to an antigen or epitope. An antibody specifically includes, e.g., intact antibodies (e.g., intact immunoglobulins, e.g., human IgG (e.g., human IgG1, human IgG2, human IgG3, human IgG4)), antibody fragments, and multi-specific antibodies. One example of an ABD is an ABD formed by a VH-VL dimer. Additional examples of an antibody are described herein. Additional examples of an antibody are known in the art.

The phrase “endosomal/lysosomal pathway” refers to a network of endosomes (early endosomes, multi-vesicular bodies, late endosomes, and lysosomes) in the cytoplasm of a mammalian cell, wherein molecules are internalized through cell-mediated internalization processes, e.g., pinocytosis, micropinocytosis, receptor-mediated endocytosis, and/or phagocytosis, are sorted.

Once the endosomes in the endosomal/lysosomal pathway are purified or isolated, assays for a target protein (e.g., an antigen-binding protein construct described herein) can be performed using methods known in the art (ELISA, Western blot, immunofluorescence, and immunoprecipitation followed by an assay for protein concentration), and can be used to determine the concentration or relative level of the target protein in the endosomes. Alternatively, endosomes in the endosomal/lysosomal pathway can be imaged using immunofluorescence microscopy using an detectably-labelled antibody (e.g., a fluorophore-labelled, a dye-labelled, or a GFP-labelled antibody, e.g., CellLight™ Early Endosome-GFP) that specifically binds to a characteristic protein present in the endosomes (e.g., EEA1 for early endosomes) and a fluorophore-labelled antibody that specifically binds to the protein of interest (e.g., an ABPC), and the level of the target protein in the endosomes can be determined by quantitation of the overlap in the fluorescence emissions of the two different antibodies.

The phrase “endolysosomal delivery” refers to rate of accumulation overtime or the total accumulation at a specific timepoint of an ABPC (e.g., any of the ABPCs described herein) in the endosomal/lysosomal pathway in a mammalian cell (e.g., any of the exemplary target mammalian cells described herein).

An exemplary method to calculate the increase in endolysosomal delivery of a pH engineered ABPC variant as compared to its corresponding starting ABPC from cellular fluorescence data is to measure the ratio of the variant's mean fluorescence intensity minus the mean fluorescence intensity of a non-binding IgG control, then all divided by the variant's corresponding starting ABPC's mean fluorescence intensity minus the mean fluorescence intensity of the IgG control.

An exemplary assay for measuring endolysosomal delivery of any of the ABPCs described herein include those which involve labeling of an ABPC with a fluorescent dye, followed by incubation of the labeled ABPC with cells and measurement of cellular fluorescence as an indicator of endolysosomal delivery of the ABPC (e.g., as described generally in Wustner, Traffic 7(6):699-715, 2006). Alternatively, pH-sensitive dyes which preferentially fluoresce at acidic pH but not neutral pH can be used to label any of the ABPCs described herein, which can then be incubated with cells and the cellular fluorescence measured as an indicator of delivery of the ABPC into acidic endolysosomal compartments.

The term “population” when used before a noun means two or more of the specific noun. For example, the phrase “a population of cancer cells” means “two or more cancer cells.” Non-limiting examples of cancer cells are described herein.

The phrase “cytostatic to a cell” refers to a direct or indirect decrease in the proliferation (cell division) of the cell (e.g., a cancer cell) in vivo or in vitro. When an agent is cytostatic to a cell, the agent can, e.g., directly or indirectly result in cell cycle arrest of the cell (e.g., a cancer cell). In some examples, an agent that is cytostatic to a cell can reduce the number of cells in a population of the cells that are in S phase (as compared to the number of cells in a population of the cells that are in S phase prior to contact with the agent). In some examples, an agent that is cytostatic to a cell can reduce the percentage of the cells in S phase by at least 20%, at least 40%, at least 60%, or at least 80% (e.g., as compared to the percentage of cells in a population of the cells that are in S phase prior to contact with the agent).

The phrase “cytotoxic to a cell” refers to the inducement, directly or indirectly, in the death (e.g., necrosis or apoptosis) of the cell (e.g., a mammalian cell, e.g., a cancer cell).

“Affinity” refers to the strength of the sum total of non-covalent interactions between an antigen-binding site and its binding partner (e.g., an antigen or epitope). Unless indicated otherwise, as used herein, “affinity” refers to intrinsic binding affinity, which reflects a 1:1 interaction between members of an ABD and an antigen or epitope. The affinity of a molecule X for its partner Y can be represented by the dissociation equilibrium constant (K_{D}). Affinity can be measured by common methods known in the art, including those described herein. Affinity can be determined, e.g., using surface plasmon resonance (SPR) technology (e.g., BIACORE®) or biolayer interferometry (e.g., FORTEBIO®). Additional methods for determining the affinity for an ABD and its corresponding antigen or epitope are known in the art.

The term “epitope” means a portion of an antigen that is specifically bound by an ABD through a set of physical interactions between: (i) all monomers (e.g. individual amino acid residues, sugar side chains, and post-translationally modified amino acid residues) on the portion of the ABD that specifically binds the antigen and (ii) all monomers (e.g. individual amino acid residues, sugar side chains, post-translationally modified amino acid residues) on the portion of the antigen that is specifically bound by the ABD. Epitopes can, e.g., consist of surface-accessible amino acid residues, sugar side chains, phosphorylated amino acid residues, methylated amino acid residues, and/or acetylated amino acid residues and may have specific three-dimensional structural characteristics, as well as specific charge characteristics. Conformational and non-conformational epitopes are distinguished in that binding to the former, but not the latter, may be lost in the presence of denaturing solvents. In some embodiments, an epitope is defined by a linear amino acid sequence of at least about 3 to 6 amino acids, or about 10 to 15 amino acids. In some embodiments, an epitope refers to a portion of a full-length protein or a portion thereof that is defined by a three-dimensional structure (e.g., protein folding). In some embodiments, an epitope is defined by a discontinuous amino acid sequence that is brought together via protein folding. In some embodiments, an epitope is defined by a discontinuous amino acid sequence that is brought together by quaternary structure (e.g., a cleft formed by the interaction of two different polypeptide chains). The amino acid sequences between the residues that define the epitope may not be critical to three-dimensional structure of the epitope. A conformational epitope may be determined and screened using assays that compare binding of antigen-binding protein construct to a denatured version of the antigen, such that a linear epitope is generated. An epitope may include amino acid residues that are directly involved in the binding, and others, which are not directly involved in the binding.

Methods for identifying an epitope to which an ABD specifically binds are known in the art, e.g., structure-based analysis (e.g. X-ray crystallography, NMR, and/or electron microscopy) (e.g. on the antigen and/or the antigen-ABD complex) and/or mutagenesis-based analysis (e.g. alanine scanning mutagenesis, glycine scanning mutagenesis, and homology scanning mutagenesis) wherein mutants are measured in a binding assay with a binding partner, many of which are known in the art.

The term “paratope” means a portion of an ABD that specifically binds to an antigen through a set of physical interactions between: (i) all monomers (e.g. individual amino acid residues, sugar side chains, posttranslationally modified amino acid residues) on the portion of the ABD that specifically binds the antigen and (ii) all monomers (e.g. individual amino acid residues, sugar side chains, posttranslationally modified amino acid residues) on the portion of the antigen that is specifically bound by the ABD. Paratopes can, e.g., consist of surface-accessible amino acid residues and may have specific three-dimensional structural characteristics, as well as specific charge characteristics. In some embodiments, a paratope refers to a portion of a full-length ABD or a portion thereof that is defined by a three-dimensional structure (e.g., protein folding). In some embodiments, a paratope is defined by a discontinuous amino acid sequence that is brought together via protein folding. In some embodiments, an epitope is defined by a discontinuous amino acid sequence that is brought together by quaternary structure (e.g., a cleft formed by the interaction of two different polypeptide chains). The amino acid sequences between the residues that define the paratope may not be critical to three-dimensional structure of the paratope. A paratope may comprise amino acid residues that are directly involved in the binding, and others, which are not directly involved in the binding.

Methods for identifying a paratope to which an ABD specifically binds are known in the art, e.g., structure-based analysis (e.g., X-ray crystallography, NMR, and/or electron microscopy) (e.g. on the ABD, and/or the ABD-antigen complex), and/or mutagenesis-based analysis (e.g., alanine scanning mutagenesis, glycine scanning mutagenesis, and homology scanning mutagenesis) wherein mutants are measured in a binding assay with a binding partner, many of which are known in the art.

The phrase “present on the surface of a mammalian cell” means (1) an antigen that physically attached to or at least partially embedded in the plasma membrane of a mammalian cell (e.g., a transmembrane protein, a peripheral membrane protein, a lipid-anchored protein (e.g., a GPI-anchor), an N-myristoylated protein, or a S-palmitoylated protein) or (2) an antigen that is stably bound to its cognate receptor, where the cognate receptor is physically attached to the plasma membrane of a cell (e.g., a ligand bound to its cognate receptor, where the cognate receptor is physically attached to the plasma membrane). Non-limiting methods for determining the presence of antigen on the surface of a mammalian cell include fluorescence-activated cell sorting (FACS), immunohistochemistry, cell-fractionation assays and Western blotting.

The phrase “control ABPC” or “control antigen-binding protein construct” means (i) an ABPC that is capable of specifically binding to TROP2 or an epitope of TROP2 presented on the surface of a mammalian cell (e.g., a target mammalian cell), where one or both of the following is true: (a) the dissociation rate of the first ABD at a pH of ~4.0 to ~6.5 (e.g., any of the subranges of this range described herein) is no more than 3-fold (e.g., no more than 2.8-fold, no more than 2.6-fold, no more than 2.5-fold, no more than 2.4-fold, no more than 2.2-fold, no more than 2.0-fold, no more than 1.8-fold, no more than 1.6-fold, no more than 1.5-fold, no more than 1.4-fold, no more than 1.2-fold, no more than 1.0-fold, no more than 0.8-fold, no more than 0.6-fold, no more than 0.5-fold, no more than 0.4-fold, no more than 0.3-fold no more than 0.2-fold, or no more than 0.1-fold) faster than the dissociation rate at a pH of ~7.0 to ~8.0 (e.g., any of the subranges of this range described herein); or (b) the dissociation constant (K_{D}) of the first ABD at a pH of ~4.0 to ~6.5 (e.g., any of the subranges of this range described herein) is no more than 3-fold (e.g., no more than 2.8-fold, no more than 2.6-fold, no more than 2.5-fold, no more than 2.4-fold, no more than 2.2-fold, no more than 2.0-fold, no more than 1.8-fold, no more than 1.6-fold, no more than 1.5-fold, no more than 1.4-fold, no more than 1.2-fold, no more than 1.0-fold, no more than 0.8-fold, no more than 0.6-fold, no more than 0.5-fold, no more than 0.4-fold, no more than 0.3-fold no more than 0.2-fold, or no more than 0.1-fold) greater than the K_{D} at a pH of ~7.0 to ~8.0 (e.g., any of the subranges of this range described herein); (ii) sacituzumab; (iii) datopotamab; (iv) hu7E6SVG; and/or (v) A1,2X4.

The term “extracellular space” means the liquid exterior to the plasma membrane of a mammalian cell. When a mammalian cell is in vitro, the extracellular space can be a liquid culture medium. When a mammalian cell is in vivo, the extracellular space can be, e.g., plasma, serum, blood, interstitial fluid, or lymph.

The term “endolysosomal space” means the fluid encapsulated by the vesicles and organelles that make-up the endosomal/lysosomal pathway in a mammalian cell.

The phrase “a reduced level” or “a decreased level” can be a reduction or decrease of at least a 1% (e.g., at least a 2%, 4%, 6%, 8%, 10%, 12%, 14%, 16%, 18%, 20%, 22%, 24%, 26%, 30%, 35%, 409%, 45%, 50%, 55%, 60%, 65%, 70%, 75%, 80%, 85%, 90%, 95%, or at least a 99%) reduction as compared to a reference level or value.

The term “cell killing potency” refers to the ability of an agent (e.g., any of the ABPCs described herein) to induce, directly or indirectly, the apoptosis and/or necrosis of a mammalian cell (e.g., a cancer cell), measured as a rate over time or at a relevant timepoint. Methods for determining the cell killing potency of a cell are known in the art (e.g., trypan blue staining, microscopy, fluorescence-assisted cell sorting, and assays to detect markers of apoptosis (e.g., Annexin V)). In non-limiting examples, cell killing potency can be measured, e.g., by cell killing at a single concentration of an agent, by the IC50 of the agent (i.e., the concentration of the agent whereby half the maximal cell killing potency is achieved), or by the ratio of an agent's dissociation constant KD on mammalian cells divided by its IC50. In some non-limiting examples, the IC50s and/or the KD ratios described herein are compared to those of a control ABPC (as defined herein), and, optionally, demonstrate that the ABPCs described herein have a higher cell killing potency as compared to the control ABPC.

The term “toxin liberation” refers to the ability of a mammalian cell (e.g., a non-cancerous mammalian cell or a cancer cell) to internalize (e.g., via pinocytosis and/or receptor-mediated endocytosis) any of the ABPCs described herein (e.g., any of ABPCs or control ABPCs described herein) that are conjugated to a toxin, and subsequently release the toxin conjugated to the ABPC, measured as a rate over time or at a specific timepoint. Toxin liberation can be assessed using a variety of different exemplary assays, e.g., ELISA, immunofluorescence, cell killing assays, cell cycle arrest assays, DNA damage assays, mass spectrometry, HPLC, and/or an isotope-labeled toxin.

The phrase “target cell” or “target mammalian cell” or “mammalian target cell” means a mammalian cell that has at least one TROP2 present on its surface. In some examples, a mammalian target cell can be a cancer cell. In some embodiments of a target mammalian cell can have a total of about the following (each±about 10%): 1-10E6, 1-9E6, 1-8E6, 1-7E6, 1-6E6, 1-5E6, 1-4E6, 1-3E6, 1-2E6, 1-1E6, 1-800,000, 1-600,000, 1-400,000, 1-200,000, 1-100,000, 1-80,000, 1-80,000, 1-75,000, 1-70,000, 1-65,000, 1-60,000, 1-55,000, 1-50,000, 145,000, 1-40,000, 1-35,000, 1-30,000, 1-25,000, 1-20,000, 1-15,000, 1-10,000, 1-7,500, 1-5,000, 1-4,000, 1-3,000, 1-2,000, 1-1,000, 1-500, 1-100, 1-50, or 1-10, or any of the ranges of numbers recited in paragraph [0065] of US 2022/0281984, of the TROP2 present on the plasma membrane of the target mammalian cell.

The phrase “antigen density” means the number of TROP2 present on the surface of a target mammalian cell or the average number of TROP2 on the surface of a population of particular type of target mammalian cells. It can be measured, e.g., using the Quantibright bead kit or radiolabel (e.g., BD Biosciences PE Phycoerythrin Fluorescence Quantitation Kit, catalog #340495).

The phrase “amino acid substituted with a histidine” means the substitution of an amino acid residue that is not histidine in a reference polypeptide sequence with a histidine. Non-limiting methods for substituting an amino acid residue in a reference polypeptide with a histidine are described herein. Additional methods for substituting an amino acid residue in a reference polypeptide with a histidine are known in the art.

The phrase “amino acid substituted with an alanine” means the substitution of an amino acid residue that is a histidine in a reference polypeptide sequence with an alanine. Non-limiting methods for substituting a histidine in a reference polypeptide with an alanine are described herein. Additional methods for substituting a histidine in a reference polypeptide with an alanine are known in the art.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure.

FIGS. 1A-1B Present octet plots for sacituzumab and its heavy chain (HC) and light chain (LC) variants (MYT11566-MYT11621). 1A: Y axes are fixed; 1B: Y axes are automatically applied to maximize vertical separation. Grey and black lines correspond to pH 5.4 and pH 7.4, respectively.

FIGS. 2A-2B Present octet plots for sacituzumab and several of its LC variants (MYT11566, MYT11622-MYT11635) and datopotamab and its HC variants (MYT11622-MYT11676). 2A: Y axes are fixed; 2B: Y axes are automatically applied to maximize vertical separation. Grey and black lines correspond to pH 5.4 and pH 7.4, respectively.

FIGS. 3A-3B Present octet plots for additional HC and LC variants (MYT11635-MYT11755). 3A: X & Y axes are fixed; 3B: X & Y axes are automatically applied to maximize vertical separation. Grey and black lines correspond to pH 5.4 and pH 7.4, respectively.

FIGS. 4A-4B Present octet plots for additional HC and LC variants (MYT12389-MYT12425). 4A: X & Y axes are fixed; 4B: X & Y axes are automatically applied to maximize vertical separation. Grey and black lines correspond to pH 5.4 and pH 7.4, respectively.

FIGS. 5A-5B Present octet plots for additional HC and LC variants (MYT12405-MYT12473). 5A: X & Y axes are fixed; 5B: X & Y axes are automatically applied to maximize vertical separation. Grey and black lines correspond to pH 5.4 and pH 7.4, respectively.

FIGS. 6A-6B Present octet plots for additional HC and LC variants (MYT12385-MYT12409). 6A: X & Y axes are fixed; 6B: X & Y axes are automatically applied to maximize vertical separation. Grey and black lines correspond to pH 5.4 and pH 7.4, respectively.

FIGS. 7A-7B Present octet plots for additional HC and LC variants (MYT12296-MYT12465). 7A: X & Y axes are fixed; 7B: X & Y axes are automatically applied to maximize vertical separation. Grey and black lines correspond to pH 5.4 and pH 7.4, respectively.

FIGS. 8A-8B Present octet plots for additional HC and LC variants (MYT12294-MYT12456). 8A: X & Y axes are fixed; 8B: X & Y axes are automatically applied to maximize vertical separation. Grey and black lines correspond to pH 5.4 and pH 7.4, respectively.

FIG. 9 is a graph showing the results of the internalization assay. Median Fluorescent Intensity (MFI) is shown for the indicated IgG constructs.

FIG. 10 is a graph showing MFI for the indicated IgG constructs.

FIG. 11 is a graph showing cytotoxicity against NCI-H1975 cells as a function of fold over datopotamab (FoD) for the indicated antibodies.

FIG. 12 is a graph showing the depth of killing of NCI-H1975 cells as a function of FoD for the indicated antibodies.

FIGS. 13A-13B Present octet plots for variants MYT13160-MYT13203. 13A: X & Y axes are fixed; 13B: X & Y axes are automatically applied to maximize vertical separation. Grey and black lines correspond to pH 5.4 and pH 7.4, respectively.

FIGS. 14A-14B Present octet plots for variants MYT13204-MYT13257. 14A: X & Y axes are fixed; 14B: X & Y axes are automatically applied to maximize vertical separation. Grey and black lines correspond to pH 5.4 and pH 7.4, respectively.

FIGS. 15A-15B Present octet plots for variants MYT13258-MYT13309. 15A: X & Y axes are fixed; 15B: X & Y axes are automatically applied to maximize vertical separation. Grey and black lines correspond to pH 5.4 and pH 7.4, respectively.

FIGS. 16A-16B Present octet plots for variants MYT13366-MYT13447. 16A: X & Y axes are fixed; 16B: X & Y axes are automatically applied to maximize vertical separation. Grey and black lines correspond to pH 5.4 and pH 7.4, respectively

FIGS. 17A-17B Present octet plots for linker-toxin-conjugated variants 112-DXd, 165-DXd, 166-DXd, 167-DXd, 168-DXd, 169-DXd, 170-DXd, 171-DXd, and 172-DXd. 17A: X & Y axes are fixed; 17B: X & Y axes are automatically applied to maximize vertical separation. Grey and black lines correspond to pH 5.4 and pH 7.4, respectively.

Additional advantages of the disclosure will be set forth in part in the description which follows, and in part will be obvious from the description, or can be learned by practice of the disclosure. The advantages of the disclosure will be realized and attained by means of the elements and combinations particularly pointed out in the appended claims. It is to be understood that both the foregoing general description and the following detailed description are exemplary and explanatory only and are not restrictive of the disclosure, as claimed.

### DETAILED DESCRIPTION

Provided herein are antigen-binding protein constructs (ABPCs) that include: a first ABD that is capable of specifically binding TROP2 or an epitope of TROP2 presented on the surface of a target mammalian cell, where: (a) the dissociation rate of the first ABD at a pH of about 4.0 to about 6.5 is faster than the dissociation rate at a pH of about 7.0 to about 8.0; and/or (b) the dissociation constant (KD) of the first ABD at a pH of about 4.0 to about 6.5 is greater than the KD at a pH of about 7.0 to about 8.0. In some examples of these ABPCs, the ABPC is degraded in the target mammalian cell following internalization of the ABPC by the target mammalian cell. Some examples of any of the ABPCs described herein can further include a conjugated toxin, radioisotope, drug, or small molecule (e.g., a fluorophore or dye).

Also provided are antigen-binding protein constructs (ABPCs) that include: a first ABD that is capable of specifically binding TROP2 or an epitope of TROP2 presented on the surface of a target mammalian cell; and a conjugated toxin, radioisotope, drug, or small molecule, where: (a) the dissociation rate of the first ABD at a pH of about 4.0 to about 6.5 is faster than the dissociation rate at a pH of about 7.0 to about 8.0; and/or the dissociation constant (KD) of the first ABD at a pH of about 4.0 to about 6.5 is greater than the KD at a pH of about 7.0 to about 8.0; and (b) a composition including the ABPC provides for one or more (e.g., two or three) of: an increase (e.g., a detectable increase) in toxin liberation in the target mammalian cell as compared to a composition comprising the same amount of a control ABPC; an increase (e.g., a detectable increase) in target mammalian cell killing as compared to a composition comprising the same amount of a control ABPC; and an increase (e.g., a detectable increase) in endolysosomal delivery in the target mammalian cell as compared to a composition comprising the same amount of a control ABPC.

In some examples, the first ABD includes an HCVD of sacituzumab with one or more amino acids substituted with a histidine. In some examples, the first ABD includes an LCVD of sacituzumab with one or more amino acids substituted with a histidine. In some examples, the first ABD includes an HCVD of sacituzumab with one or more amino acids substituted with a histidine; and an LCVD of sacituzumab with one or more amino acids substituted with a histidine. In some examples, the HCVD of sacituzumab comprises SEQ ID NO: 4. In some examples, the LCVD of sacituzumab comprises SEQ ID NO: 5. In each of the foregoing examples, an alanine may be substituted where a histidine was already present.

In some examples, the first ABD comprises an HCVD comprising a CDR1, a CDR2, and a CDR3 of SEQ ID NOs: 6, 7, and 8, respectively, with collectively a total of one or more amino acid positions in SEQ ID NOs: 6, 7, and 8 substituted with a histidine. In some examples, the first ABD comprises an LCVD comprising a CDR1, a CDR2, and a CDR3 of SEQ ID NOs: 9, 10, and 11, respectively, with collectively a total of one or more amino acid positions in SEQ ID NOs: 9, 10, and 11 substituted with a histidine. In some examples, the first ABD includes: an HCVD comprising a CDR1, a CDR2, and a CDR3 of SEQ ID NOs: 6, 7, and 8, respectively, with collectively a total of one or more amino acid positions in NOs: 6, 7, and 8 substituted with a histidine; and an LCVD comprising a CDR1, a CDR2, and a CDR3 of SEQ ID NOs: 9, 10, and 11, respectively, with collectively a total of one or more amino acid positions in SEQ ID NOs: 9, 10, and 11 substituted with a histidine. In each of the foregoing examples, an alanine may be substituted where a histidine was already present.

In some examples, the first ABD includes an HCVD that is at least 90%, 95%, or 98% identical to SEQ ID NO: 4, where the HCVD includes a histidine at one or more amino acid positions in SEQ ID NO: 4 selected from: 29, 32, 33, 50, 52, 53, 60, 61, 62, 63, 64, 65, 66, 99, 101, 104, 105, and 108. In some examples, the first ABD includes an LCVD that is at least 90%, 95%, or 98% identical to SEQ ID NO: 5, where the LCVD includes a histidine at one or more amino acid positions in SEQ ID NO: 5 selected from: 28, 29, 30, 31, 32, 33, 50, 51, 52, 53, 89, 92, 93, 94, 95, 96, and 97. In some examples, the first ABD includes an HCVD that is at least 90%, 95%, or 98% identical to SEQ ID NO: 4, where the HCVD includes a histidine at one or more amino acid positions in SEQ ID NO: 4 selected from: 29, 32, 33, 50, 52, 53, 60, 61, 62, 63, 64, 65, 66, 99, 101, 104, 105, and 108, and an LCVD that is at least 90%, 95%, or 98% identical to SEQ ID NO: 5, where the LCVD includes a histidine at one or more amino acid positions in SEQ ID NO: 5 selected from: 28, 29, 30, 31, 32, 33, 50, 51, 52, 53, 89, 92, 93, 94, 95, 96, and 97.

In some examples, an HCVD includes an HCVD that is at least 90%, 95%, or 98% identical to SEQ ID NO: 4, where the HCVD includes a histidine at any of the specific combinations of two or more amino acid positions in SEQ ID NO: 4 listed in Table 1.

TABLE 1
Exemplary Combinations of Amino Acid Positions in SEQ ID NO: 4 that can be substituted

| | Column | | | | | | | | | | | | | | | | | |
|---|---|---|---|---|---|---|---|---|---|---|---|---|---|---|---|---|---|---|
| | 1 | 2 | 3 | 4 | 5 | 6 | 7 | 8 | 9 | 10 | 11 | 12 | 13 | 14 | 15 | 16 | 17 | 18 |
| SEQ 4 AA Position | 29 | 32 | 33 | 50 | 52 | 53 | 60 | 61 | 62 | 63 | 64 | 65 | 66 | 99 | 101 | 104 | 105 | 108 |

Exemplary 2, 3, and 4 amino acid position combinations that can be substituted with histidine

| | |
|---|---|
| A | Each and every unique 2-position combination of X and Y, wherein X and Y are each selected |
| | from a SEQ ID NO: 4 amino acid position recited in one of columns 1 to 18, without |
| | replacement (i.e., X ≠ Y). Expressed another way, the combinations of positions recited in the |
| | following combinations of columns: 1, 2, 1 + 3, 1 + 4, 1 + 5, 1 + 6, 1 + 7, 1 + 8, 1 + 9, 1 + 10, 1 + 11, 1 + 12, |
| | 1 + 13, 1 + 14, 1 + 15, 1 + 16, 1 + 17, 1 + 18, 2 + 3, 2 + 4, 2 + 5, 2 + 6, 2 + 7, 2 + 8, 2 + 9, 2 + 10, 2 + 11, 2 + 12, |
| | 2 + 13, 2 + 14, 2 + 15, 2 + 16, 2 + 17, 2 + 18, 3 + 4, 3 + 5, 3 + 6, 3 + 7, 3 + 8, 3 + 9, 3 + 10, 3 + 11, 3 + 12, 3 + 13, |
| | 3 + 14, 3 + 15, 3 + 16, 3 + 17, 3 + 18, 4 + 5, 4 + 6, 4 + 7, 4 + 8, 4 + 9, 4 + 10, 4 + 11, 4 + 12, 4 + 13, 4 + 14, 4 + 15, |
| | 4 + 16, 4 + 17, 4 + 18, 5 + 6, 5 + 7, 5 + 8, 5 + 9, 5 + 10, 5 + 11, 5 + 12, 5 + 13, 5 + 14, 5 + 15, 5 + 16, 5 + 17, |
| | 5 + 18, 6 + 7, 6 + 8, 6 + 9, 6 + 10, 6 + 11, 6 + 12, 6 + 13, 6 + 14, 6 + 15, 6 + 16, 6 + 17, 6 + 18, 7 + 8, 7 + 9, 7 + 10, |
| | 7 + 11, 7 + 12, 7 + 13, 7 + 14, 7 + 15, 7 + 16, 7 + 17, 7 + 18, 8 + 9, 8 + 10, 8 + 11, 8 + 12, 8 + 13, 8 + 14, 8 + 15, |
| | 8 + 16, 8 + 17, 8 + 18, 9 + 10, 9 + 11, 9 + 12, 9 + 13, 9 + 14, 9 + 15, 9 + 16, 9 + 17, 9 + 18, 10 + 11, 10 + 12, |
| | 10 + 13, 10 + 14, 10 + 15, 10 + 16, 10 + 17, 10 + 18, 11 + 12, 11 + 13, 11 + 14, 11 + 15, 11 + 16, 11 + 17, |
| | 11 + 18, 12 + 13, 12 + 14, 12 + 15, 12 + 16, 12 + 17, 12 + 18, 13 + 14, 13 + 15, 13 + 16, 13 + 17, 13 + 18, |
| | 14 + 15, 14 + 16, 14 + 17, 14 + 18, 15 + 16, 15 + 17, 15 + 18, 16 + 17, 16 + 18, and 17 + 18. |
| B | Each and every unique 3-position combination of X, Y, and Z, wherein X and Y are together |
| | one of the 2-position combinations recited in “A” (above), and Z is selected from a SEQ ID |
| | NO: 4 amino acid position recited in one of columns 1 to 18, without replacement (i.e., |
| | X ≠ Y ≠ Z). Expressed another way, the combinations of positions recited in the following |
| | combinations of columns: 1 + 2 + 3, 1 + 2 + 4, 1 + 2 + 5, 1 + 2 + 6, 1 + 2 + 7, 1 + 2 + 8, 1 + 2 + 9, 1 + 2 + 10, |
| | 1 + 2 + 11, 1 + 2 + 12, 1 + 2 + 13, 1 + 2 + 14, 1 + 2 + 15, 1 + 2 + 16, 1 + 2 + 17, 1 + 2 + 18, 1 + 3 + 4, 1 + 3 + 5, 1 + 3 + 6, |
| | 1 + 3 + 7, 1 + 3 + 8, 1 + 3 + 9, 1 + 3 + 10, 1 + 3 + 11, 1 + 3 + 12, 1 + 3 + 13, 1 + 3 + 14, 1 + 3 + 15, 1 + 3 + 16, 1 + 3 + 17, |
| | 1 + 3 + 18, 1 + 4 + 5, 1 + 4 + 6, 1 + 4 + 7, 1 + 4 + 8, 1 + 4 + 9, 1 + 4 + 10, 1 + 4 + 11, 1 + 4 + 12, 1 + 4 + 13, 1 + 4 + 14, |
| | 1 + 4 + 15, 1 + 4 + 16, 1 + 4 + 17, 1 + 4 + 18, 1 + 5 + 6, 1 + 5 + 7, 1 + 5 + 8, 1 + 5 + 9, 1 + 5 + 10, 1 + 5 + 11, 1 + 5 + 12, |
| | 1 + 5 + 13, 1 + 5 + 14, 1 + 5 + 15, 1 + 5 + 16, 1 + 5 + 17, 1 + 5 + 18, 1 + 6 + 7, 1 + 6 + 8, 1 + 6 + 9, 1 + 6 + 10, 1 + 6 + 11, |
| | 1 + 6 + 12, 1 + 6 + 13, 1 + 6 + 14, 1 + 6 + 15, 1 + 6 + 16, 1 + 6 + 17, 1 + 6 + 18, 1 + 7 + 8, 1 + 7 + 9, 1 + 7 + 10, |
| | 1 + 7 + 11. 1 + 7 + 12, 1 + 7 + 13, 1 + 7 + 14, 1 + 7 + 15, 1 + 7 + 16, 1 + 7 + 17, 1 + 7 + 18, 1 + 8 + 9, 1 + 8 + 10, |
| | 1 + 8 + 11, 1 + 8 + 12, 1 + 8 + 13, 1 + 8 + 14, 1 + 8 + 15, 1 + 8 + 16, 1 + 8 + 17, 1 + 8 + 18, 1 + 9 + 10, 1 + 9 + 11, |
| | 1 + 9 + 12, 1 + 9 + 13, 1 + 9 + 14, 1 + 9 + 15, 1 + 9 + 16, 1 + 9 + 17, 1 + 9 + 18, 1 + 10 + 11, 1 + 10 + 12, 1 + 10 + 13, |
| | 1 + 10 + 14, 1 + 10 + 15, 1 + 10 + 16, 1 + 10 + 17, 1 + 10 + 18, 1 + 11 + 12, 1 + 11 + 13, 1 + 11 + 14, 1 + 11 + 15, |
| | 1 + 11 + 16, 1 + 11 + 17, 1 + 11 + 18, 1 + 12 + 13, 1 + 12 + 14, 1 + 12 + 15, 1 + 12 + 16, 1 + 12 + 17, 1 + 12 + 18, |
| | 1 + 13 + 14, 1 + 13 + 15, 1 + 13 + 16, 1 + 13 + 17, 1 + 13 + 18, 1 + 14 + 15, 1 + 14 + 16, 1 + 14 + 17, 1 + 14 + 18, |
| | 1 + 15 + 16, 1 + 15 + 17, 1 + 15 + 18, 1 + 16 + 17, 1 + 16 + 18, 1 + 17 + 18, 2 + 3 + 4, 2 + 3 + 5, 2 + 3 + 6, 2 + 3 + 7, |
| | 2 + 3 + 8, 2 + 3 + 9, 2 + 3 + 10, 2 + 3 + 11, 2 + 3 + 12, 2 + 3 + 13, 2 + 3 + 14, 2 + 3 + 15, 2 + 3 + 16, 2 + 3 + 17, |
| | 2 + 3 + 18, 2 + 4 + 5, 2 + 4 + 6, 2 + 4 + 7, 2 + 4 + 8, 2 + 4 + 9, 2 + 4 + 10, 2 + 4 + 11, 2 + 4 + 12, 2 + 4 + 13, 2 + 4 + 14, |
| | 2 + 4 + 15, 2 + 4 + 16, 2 + 4 + 17, 2 + 4 + 18, 2 + 5 + 6, 2 + 5 + 7, 2 + 5 + 8, 2 + 5 + 9, 2 + 5 + 10, 2 + 5 + 11, 2 + 5 + 12, |
| | 2 + 5 + 13, 2 + 5 + 14, 2 + 5 + 15, 2 + 5 + 16, 2 + 5 + 17, 2 + 5 + 18, 2 + 6 + 7, 2 + 6 + 8, 2 + 6 + 9, 2 + 6 + 10, 2 + 6 + 11, |
| | 2 + 6 + 12, 2 + 6 + 13, 2 + 6 + 14, 2 + 6 + 15, 2 + 6 + 16, 2 + 6 + 17, 2 + 6 + 18, 2 + 7 + 8, 2 + 7 + 9, 2 + 7 + 10, |
| | 2 + 7 + 11, 2 + 7 + 12, 2 + 7 + 13, 2 + 7 + 14, 2 + 7 + 15, 2 + 7 + 16, 2 + 7 + 17, 2 + 7 + 18, 2 + 8 + 9, 2 + 8 + 10, |
| | 2 + 8 + 11, 2 + 8 + 12, 2 + 8 + 13, 2 + 8 + 14, 2 + 8 + 15, 2 + 8 + 16, 2 + 8 + 17, 2 + 8 + 18, 2 + 9 + 10, 2 + 9 + 11, |
| | 2 + 9 + 12, 2 + 9 + 13, 2 + 9 + 14, 2 + 9 + 15, 2 + 9 + 16, 2 + 9 + 17, 2 + 9 + 18, 2 + 10 + 11, 2 + 10 + 12, 2 + 10 + 13, |
| | 2 + 10 + 14, 2 + 10 + 15, 2 + 10 + 16, 2 + 10 + 17, 2 + 10 + 18, 2 + 11 + 12, 2 + 11 + 13, 2 + 11 + 14, 2 + 11 + 15, |
| | 2 + 11 + 16, 2 + 11 + 17, 2 + 11 + 18, 2 + 12 + 13, 2 + 12 + 14, 2 + 12 + 15, 2 + 12 + 16, 2 + 12 + 17, 2 + 12 + 18, |
| | 2 + 13 + 14, 2 + 13 + 15, 2 + 13 + 16, 2 + 13 + 17, 2 + 13 + 18, 2 + 14 + 15, 2 + 14 + 16, 2 + 14 + 17, 2 + 14 + 18, |
| | 2 + 15 + 16, 2 + 15 + 17, 2 + 15 + 18, 2 + 16 + 17, 2 + 16 + 18, 2 + 17 + 18, 3 + 4 + 5, 3 + 4 + 6, 3 + 4 + 7, 3 + 4 + 8, |
| | 3 + 4 + 9, 3 + 4 + 10, 3 + 4 + 11, 3 + 4 + 12, 3 + 4 + 13, 3 + 4 + 14, 3 + 4 + 15, 3 + 4 + 16, 3 + 4 + 17, 3 + 4 + 18, |
| | 3 + 5 + 6, 3 + 5 + 7, 3 + 5 + 8, 3 + 5 + 9, 3 + 5 + 10, 3 + 5 + 11, 3 + 5 + 12, 3 + 5 + 13, 3 + 5 + 14, 3 + 5 + 15, 3 + 5 + 16, |
| | 3 + 5 + 17, 3 + 5 + 18, 3 + 6 + 7, 3 + 6 + 8, 3 + 6 + 9, 3 + 6 + 10, 3 + 6 + 11, 3 + 6 + 12, 3 + 6 + 13, 3 + 6 + 14, 3 + 6 + 15, |
| | 3 + 6 + 16, 3 + 6 + 17, 3 + 6 + 18, 3 + 7 + 8, 3 + 7 + 9, 3 + 7 + 10, 3 + 7 + 11, 3 + 7 + 12, 3 + 7 + 13, 3 + 7 + 14, |
| | 3 + 7 + 15, 3 + 7 + 16, 3 + 7 + 17, 3 + 7 + 18, 3 + 8 + 9, 3 + 8 + 10, 3 + 8 + 11, 3 + 8 + 12, 3 + 8 + 13, 3 + 8 + 14, |
| | 3 + 8 + 15, 3 + 8 + 16, 3 + 8 + 17, 3 + 8 + 18, 3 + 9 + 10, 3 + 9 + 11, 3 + 9 + 12, 3 + 9 + 13, 3 + 9 + 14, 3 + 9 + 15, |
| | 3 + 9 + 16, 3 + 9 + 17, 3 + 9 + 18, 3 + 10 + 11, 3 + 10 + 12, 3 + 10 + 13, 3 + 10 + 14, 3 + 10 + 15, 3 + 10 + 16, |
| | 3 + 10 + 17, 3 + 10 + 18, 3 + 11 + 12, 3 + 11 + 13, 3 + 11 + 14, 3 + 11 + 15, 3 + 11 + 16, 3 + 11 + 17, 3 + 11 + 18, |
| | 3 + 12 + 13, 3 + 12 + 14, 3 + 12 + 15, 3 + 12 + 16, 3 + 12 + 17, 3 + 12 + 18, 3 + 13 + 14, 3 + 13 + 15, 3 + 13 + 16, |
| | 3 + 13 + 17, 3 + 13 + 18, 3 + 14 + 15, 3 + 14 + 16, 3 + 14 + 17, 3 + 14 + 18, 3 + 15 + 16, 3 + 15 + 17, 3 + 15 + 18, |
| | 3 + 16 + 17, 3 + 16 + 18, 3 + 17 + 18, 4 + 5 + 6, 4 + 5 + 7, 4 + 5 + 8, 4 + 5 + 9, 4 + 5 + 10, 4 + 5 + 11, 4 + 5 + 12, |
| | 4 + 5 + 13, 4 + 5 + 14, 4 + 5 + 15, 4 + 5 + 16, 4 + 5 + 17, 4 + 5 + 18, 4 + 6 + 7, 4 + 6 + 8, 4 + 6 + 9, 4 + 6 + 10, 4 + 6 + 11, |
| | 4 + 6 + 12, 4 + 6 + 13, 4 + 6 + 14, 4 + 6 + 15, 4 + 6 + 16, 4 + 6 + 17, 4 + 6 + 18, 4 + 7 + 8, 4 + 7 + 9, 4 + 7 + 10, |
| | 4 + 7 + 11, 4 + 7 + 12, 4 + 7 + 13, 4 + 7 + 14, 4 + 7 + 15, 4 + 7 + 16, 4 + 7 + 17, 4 + 7 + 18, 4 + 8 + 9, 4 + 8 + 10, |
| | 4 + 8 + 11, 4 + 8 + 12, 4 + 8 + 13, 4 + 8 + 14, 4 + 8 + 15, 4 + 8 + 16, 4 + 8 + 17, 4 + 8 + 18, 4 + 9 + 10, 4 + 9 + 11, |
| | 4 + 9 + 12, 4 + 9 + 13, 4 + 9 + 14, 4 + 9 + 15, 4 + 9 + 16, 4 + 9 + 17, 4 + 9 + 18, 4 + 10 + 11, 4 + 10 + 12, 4 + 10 + 13, |
| | 4 + 10 + 14, 4 + 10 + 15, 4 + 10 + 16, 4 + 10 + 17, 4 + 10 + 18, 4 + 11 + 12, 4 + 11 + 13, 4 + 11 + 14, 4 + 11 + 15, |
| | 4 + 11 + 16, 4 + 11 + 17, 4 + 11 + 18, 4 + 12 + 13, 4 + 12 + 14, 4 + 12 + 15, 4 + 12 + 16, 4 + 12 + 17, 4 + 12 + 18, |
| | 4 + 13 + 14, 4 + 13 + 15, 4 + 13 + 16, 4 + 13 + 17, 4 + 13 + 18, 4 + 14 + 15, 4 + 14 + 16, 4 + 14 + 17, 4 + 14 + 18, |
| | 4 + 15 + 16, 4 + 15 + 17, 4 + 15 + 18, 4 + 16 + 17, 4 + 16 + 18, 4 + 17 + 18, 5 + 6 + 7, 5 + 6 + 8, 5 + 6 + 9, 5 + 6 + 10, |
| | 5 + 6 + 11, 5 + 6 + 12, 5 + 6 + 13, 5 + 6 + 14, 5 + 6 + 15, 5 + 6 + 16, 5 + 6 + 17, 5 + 6 + 18, 5 + 7 + 8, 5 + 7 + 9, |
| | 5 + 7 + 10, 5 + 7 + 11, 5 + 7 + 12, 5 + 7 + 13, 5 + 7 + 14, 5 + 7 + 15, 5 + 7 + 16, 5 + 7 + 17, 5 + 7 + 18, 5 + 8 + 9, |
| | 5 + 8 + 10, 5 + 8 + 11, 5 + 8 + 12, 5 + 8 + 13, 5 + 8 + 14, 5 + 8 + 15, 5 + 8 + 16, 5 + 8 + 17, 5 + 8 + 18, 5 + 9 + 10, |
| | 5 + 9 + 11, 5 + 9 + 12, 5 + 9 + 13, 5 + 9 + 14, 5 + 9 + 15, 5 + 9 + 16, 5 + 9 + 17, 5 + 9 + 18, 5 + 10 + 11, 5 + 10 + 12, |
| | 5 + 10 + 13, 5 + 10 + 14, 5 + 10 + 15, 5 + 10 + 16, 5 + 10 + 17, 5 + 10 + 18, 5 + 11 + 12, 5 + 11 + 13, 5 + 11 + 14, |
| | 5 + 11 + 15, 5 + 11 + 16, 5 + 11 + 17, 5 + 11 + 18, 5 + 12 + 13, 5 + 12 + 14, 5 + 12 + 15, 5 + 12 + 16, 5 + 12 + 17, |
| | 5 + 12 + 18, 5 + 13 + 14, 5 + 13 + 15, 5 + 13 + 16, 5 + 13 + 17, 5 + 13 + 18, 5 + 14 + 15, 5 + 14 + 16, 5 + 14 + 17, |
| | 5 + 14 + 18, 5 + 15 + 16, 5 + 15 + 17, 5 + 15 + 18, 5 + 16 + 17, 5 + 16 + 18, 5 + 17 + 18, 6 + 7 + 8, 6 + 7 + 9, |
| | 6 + 7 + 10, 6 + 7 + 11, 6 + 7 + 12, 6 + 7 + 13, 6 + 7 + 14, 6 + 7 + 15, 6 + 7 + 16, 6 + 7 + 17, 6 + 7 + 18, 6 + 8 + 9, |
| | 6 + 8 + 10, 6 + 8 + 11, 6 + 8 + 12, 6 + 8 + 13, 6 + 8 + 14, 6 + 8 + 15, 6 + 8 + 16, 6 + 8 + 17, 6 + 8 + 18, 6 + 9 + 10, |
| | 6 + 9 + 11, 6 + 9 + 12, 6 + 9 + 13, 6 + 9 + 14, 6 + 9 + 15, 6 + 9 + 16, 6 + 9 + 17, 6 + 9 + 18, 6 + 10 + 11, 6 + 10 + 12, |
| | 6 + 10 + 13, 6 + 10 + 14, 6 + 10 + 15, 6 + 10 + 16, 6 + 10 + 17, 6 + 10 + 18, 6 + 11 + 12, 6 + 11 + 13, 6 + 11 + 14, |
| | 6 + 11 + 15, 6 + 11 + 16, 6 + 11 + 17, 6 + 11 + 18, 6 + 12 + 13, 6 + 12 + 14, 6 + 12 + 15, 6 + 12 + 16, 6 + 12 + 17, |
| | 6 + 12 + 18, 6 + 13 + 14, 6 + 13 + 15, 6 + 13 + 16, 6 + 13 + 17, 6 + 13 + 18, 6 + 14 + 15, 6 + 14 + 16, 6 + 14 + 17, |
| | 6 + 14 + 18, 6 + 15 + 16, 6 + 15 + 17, 6 + 15 + 18, 6 + 16 + 17, 6 + 16 + 18, 6 + 17 + 18, 7 + 8 + 9, 7 + 8 + 10, |
| | 7 + 8 + 11, 7 + 8 + 12, 7 + 8 + 13, 7 + 8 + 14, 7 + 8 + 15, 7 + 8 + 16, 7 + 8 + 17, 7 + 8 + 18, 7 + 9 + 10, 7 + 9 + 11, |
| | 7 + 9 + 12, 7 + 9 + 13, 7 + 9 + 14, 7 + 9 + 15, 7 + 9 + 16, 7 + 9 + 17, 7 + 9 + 18, 7 + 10 + 11, 7 + 10 + 12, 7 + 10 + 13, |
| | 7 + 10 + 14, 7 + 10 + 15, 7 + 10 + 16, 7 + 10 + 17, 7 + 10 + 18, 7 + 11 + 12, 7 + 11 + 13, 7 + 11 + 14, 7 + 11 + 15, |
| | 7 + 11 + 16, 7 + 11 + 17, 7 + 11 + 18, 7 + 12 + 13, 7 + 12 + 14, 7 + 12 + 15, 7 + 12 + 16, 7 + 12 + 17, 7 + 12 + 18, |
| | 7 + 13 + 14, 7 + 13 + 15, 7 + 13 + 16, 7 + 13 + 17, 7 + 13 + 18, 7 + 14 + 15, 7 + 14 + 16, 7 + 14 + 17, 7 + 14 + 18, |
| | 7 + 15 + 16, 7 + 15 + 17, 7 + 15 + 18, 7 + 16 + 17, 7 + 16 + 18, 7 + 17 + 18, 8 + 9 + 10, 8 + 9 + 11, 8 + 9 + 12, |
| | 8 + 9 + 13, 8 + 9 + 14, 8 + 9 + 15, 8 + 9 + 16, 8 + 9 + 17, 8 + 9 + 18, 8 + 10 + 11, 8 + 10 + 12, 8 + 10 + 13, 8 + 10 + 14, |
| | 8 + 10 + 15, 8 + 10 + 16, 8 + 10 + 17, 8 + 10 + 18, 8 + 11 + 12, 8 + 11 + 13, 8 + 11 + 14, 8 + 11 + 15, 8 + 11 + 16, |
| | 8 + 11 + 17, 8 + 11 + 18, 8 + 12 + 13, 8 + 12 + 14, 8 + 12 + 15, 8 + 12 + 16, 8 + 12 + 17, 8 + 12 + 18, 8 + 13 + 14, |
| | 8 + 13 + 15, 8 + 13 + 16, 8 + 13 + 17, 8 + 13 + 18, 8 + 14 + 15, 8 + 14 + 16, 8 + 14 + 17, 8 + 14 + 18, 8 + 15 + 16, |
| | 8 + 15 + 17, 8 + 15 + 18, 8 + 16 + 17, 8 + 16 + 18, 8 + 17 + 18, 9 + 10 + 11, 9 + 10 + 12, 9 + 10 + 13, 9 + 10 + 14, |
| | 9 + 10 + 15, 9 + 10 + 16, 9 + 10 + 17, 9 + 10 + 18, 9 + 11 + 12, 9 + 11 + 13, 9 + 11 + 14, 9 + 11 + 15, 9 + 11 + 16, |
| | 9 + 11 + 17, 9 + 11 + 18, 9 + 12 + 13, 9 + 12 + 14, 9 + 12 + 15, 9 + 12 + 16, 9 + 12 + 17, 9 + 12 + 18, 9 + 13 + 14, |
| | 9 + 13 + 15, 9 + 13 + 16, 9 + 13 + 17, 9 + 13 + 18, 9 + 14 + 15, 9 + 14 + 16, 9 + 14 + 17, 9 + 14 + 18, 9 + 15 + 16, |
| | 9 + 15 + 17, 9 + 15 + 18, 9 + 16 + 17, 9 + 16 + 18, 9 + 17 + 18, 10 + 11 + 12, 10 + 11 + 13, 10 + 11 + 14, |
| | 10 + 11 + 15, 10 + 11 + 16, 10 + 11 + 17, 10 + 11 + 18, 10 + 12 + 13, 10 + 12 + 14, 10 + 12 + 15, 10 + 12 + 16, |
| | 10 + 12 + 17, 10 + 12 + 18, 10 + 13 + 14, 10 + 13 + 15, 10 + 13 + 16, 10 + 13 + 17, 10 + 13 + 18, 10 + 14 + 15, |
| | 10 + 14 + 16, 10 + 14 + 17, 10 + 14 + 18, 10 + 15 + 16, 10 + 15 + 17, 10 + 15 + 18, 10 + 16 + 17, 10 + 16 + 18, |
| | 10 + 17 + 18, 11 + 12 + 13, 11 + 12 + 14, 11 + 12 + 15, 11 + 12 + 16, 11 + 12 + 17, 11 + 12 + 18, 11 + 13 + 14, |
| | 11 + 13 + 15, 11 + 13 + 16, 11 + 13 + 17, 11 + 13 + 18, 11 + 14 + 15, 11 + 14 + 16, 11 + 14 + 17, 11 + 14 + 18, |
| | 11 + 15 + 16, 11 + 15 + 17, 11 + 15 + 18, 11 + 16 + 17, 11 + 16 + 18, 11 + 17 + 18, 12 + 13 + 14, 12 + 13 + 15, |
| | 12 + 13 + 16, 12 + 13 + 17, 12 + 13 + 18, 12 + 14 + 15, 12 + 14 + 16, 12 + 14 + 17, 12 + 14 + 18, 12 + 15 + 16, |
| | 12 + 15 + 17, 12 + 15 + 18, 12 + 16 + 17, 12 + 16 + 18, 12 + 17 + 18, 13 + 14 + 15, 13 + 14 + 16, 13 + 14 + 17, |
| | 13 + 14 + 18, 13 + 15 + 16, 13 + 15 + 17, 13 + 15 + 18, 13 + 16 + 17, 13 + 16 + 18, 13 + 17 + 18, 14 + 15 + 16, |
| | 14 + 15 + 17, 14 + 15 + 18, 14 + 16 + 17, 14 + 16 + 18, 14 + 17 + 18, 15 + 16 + 17, 15 + 16 + 18, 15 + 17 + 18, |
| | and 16 + 17 + 18. |
| C | Each and every unique 4-position combination of X & Y & Z & α, wherein X, Y, and Z are |
| | together one of the 3-position combinations recited in “B” (above), and α is selected from a |
| | SEQ ID NO: 4 amino acid position recited in one of columns 1 to 18, without replacement (i.e. |
| | X ≠ Y ≠ Z ≠ α). |

In some examples, the first ABD includes an LCVD that is at least 90%, 95%, or 98% identical to SEQ ID NO. 5, where the LCVD includes a histidine at any of the specific combinations of two or more amino acid positions in SEQ ID NO: 5 listed in Table 2 and/or where the LCVD includes an alanine at position 91 of SEQ ID NO: 5.

In some examples, the first ABD includes an LCVD that is at least 90%, 95%, or 98% identical to SEQ ID NO: 5, where the LCVD includes an alanine at position 91 in SEQ ID NO: 5.

In some examples the first ABD includes: an LCVD that is at least 90%, 95%, or 98% identical to SEQ ID NO: 5, where the LCVD includes an alanine at position 91 in SEQ ID NO: 5, and an HCVD that is at least 90%, 95%, or 98% identical to SEQ ID NO: 4, where the HCVD includes a histidine at one or more amino acid positions in SEQ ID NO: 4 selected from: 29, 32, 33, 50, 52, 53, 60, 61, 62, 63, 64, 65, 66, 99, 101, 104, 105, and 108.

In some examples, the first ABD includes an LCVD that is at least 90%, 95%, or 98% identical to SEQ ID NO: 5, where the LCVD includes a histidine at one or more amino acid positions in SEQ ID NO: 5 selected from. 28, 29, 30, 31, 32, 33, 50, 51, 52, 53, 89, 92, 93, 94, 95, 96, and 97 and/or where the LCVD includes an alanine at position 91 in SEQ ID NO: 5. In some examples the first ABD includes: an LCVD that is at least 90%, 95%, or 98% identical to SEQ ID NO: 5, where the LCVD includes a histidine at one or more amino acid positions in SEQ ID NO. 5 selected from: 28, 29, 30, 31, 32, 33, 50, 51, 52, 53, 89, 92, 93, 94, 95, 96, and 97 and/or where the LCVD includes an alanine at position 91 in SEQ ID NO: 5, and an HCVD that is at least 90%, 95%, or 98% identical to SEQ ID NO: 4, where the HCVD includes a histidine at one or more amino acid positions in SEQ ID NO: 4 selected from: 29, 32, 33, 50, 52, 53, 60, 61, 62, 63, 64, 65, 66, 99, 101, 104, 105, and 108.

In some examples the first ABD includes an LCVD that is at least 90%, 95%, or 98% identical to SEQ ID NO: 5, where the LCVD includes a histidine at one or more amino acid positions in SEQ ID NO: 5 selected from: 28, 29, 30, 31, 32, 33, 50, 51, 52, 53, 89, 92, 93, 94, 95, 96, and 97 and/or where the LCVD includes an alanine at position 91 in SEQ ID NO: 5, and an HCVD that is at least 90%, 95%, or 98% identical to SEQ ID NO: 4, where the HCVD includes a histidine at one or more amino acid positions in SEQ ID NO: 4 selected from: 29, 32, 33, 50, 52, 53, 60, 61, 62, 63, 64, 65, 66, 99, 101, 104, 105, and 108 and/or where the HCVD includes an alanine at position 91.

TABLE 2
Exemplary Combinations of Amino Acid Positions in SEQ ID NO: 5 that can
be Substituted with Histidine (or Alanine (A) in the case of position 91)

| | Column | | | | | | | | | | | | | | | | | |
|---|---|---|---|---|---|---|---|---|---|---|---|---|---|---|---|---|---|---|
| | 1 | 2 | 3 | 4 | 5 | 6 | 7 | 8 | 9 | 10 | 11 | 12 | 13 | 14 | 15 | 16 | 17 | 18 |
| SEQ 5 AA Position | 28 | 29 | 30 | 31 | 32 | 33 | 50 | 51 | 52 | 53 | 89 | 92 | 93 | 94 | 95 | 96 | 97 | 91 (A) |

Exemplary 2, 3, and 4 AA position combinations that can be substituted with histidine
(or an alanine at position 91 of SEQ ID NO: 5)

| | |
|---|---|
| A | Each and every unique 2-position combination of X & Y, wherein X and Y are each selected |
| | (without replacement) from a SEQ ID NO: 5 AA position indicated in columns 1-18. |
| | Expressed another way, the combinations of positions recited in the following combinations |
| | of columns: 1, 2, 1 + 3, 1 + 4, 1 + 5, 1 + 6, 1 + 7, 1 + 8, 1 + 9, 1 + 10, 1 + 11, 1 + 12, 1 + 13, 1 + 14, 1 + 15, |
| | 1 + 16, 1 + 17, 1 + 18, 2 + 3, 2 + 4, 2 + 5, 2 + 6, 2 + 7, 2 + 8, 2 + 9, 2 + 10, 2 + 11, 2 + 12, 2 + 13, 2 + 14, |
| | 2 + 15, 2 + 16, 2 + 17, 2 + 18, 3 + 4, 3 + 5, 3 + 6, 3 + 7, 3 + 8, 3 + 9, 3 + 10, 3 + 11, 3 + 12, 3 + 13, 3 + 14, |
| | 3 + 15, 3 + 16, 3 + 17, 3 + 18, 4 + 5, 4 + 6, 4 + 7, 4 + 8, 4 + 9, 4 + 10, 4 + 11, 4 + 12, 4 + 13, 4 + 14, 4 + 15, |
| | 4 + 16, 4 + 17, 4 + 18, 5 + 6, 5 + 7, 5 + 8, 5 + 9, 5 + 10, 5 + 11, 5 + 12, 5 + 13, 5 + 14, 5 + 15, 5 + 16, 5 + 17, |
| | 5 + 18, 6 + 7, 6 + 8, 6 + 9, 6 + 10, 6 + 11, 6 + 12, 6 + 13, 6 + 14, 6 + 15, 6 + 16, 6 + 17, 6 + 18, 7 + 8, 7 + 9, |
| | 7 + 10, 7 + 11, 7 + 12, 7 + 13, 7 + 14, 7 + 15, 7 + 16, 7 + 17, 7 + 18, 8 + 9, 8 + 10, 8 + 11, 8 + 12, 8 + 13, |
| | 8 + 14, 8 + 15, 8 + 16, 8 + 17, 8 + 18, 9 + 10, 9 + 11, 9 + 12, 9 + 13, 9 + 14, 9 + 15, 9 + 16, 9 + 17, 9 + 18, |
| | 10 + 11, 10 + 12, 10 + 13, 10 + 14, 10 + 15, 10 + 16, 10 + 17, 10 + 18, 11 + 12, 11 + 13, 11 + 14, 11 + 15, |
| | 11 + 16, 11 + 17, 11 + 18, 12 + 13, 12 + 14, 12 + 15, 12 + 16, 12 + 17, 12 + 18, 13 + 14, 13 + 15, 13 + 16, |
| | 13 + 17, 13 + 18, 14 + 15, 14 + 16, 14 + 17, 14 + 18, 15 + 16, 15 + 17, 15 + 18, 16 + 17, 16 + 18, and |
| | 17 + 18. |
| B | Each and every unique 3-position combination of X & Y & Z, wherein X, Y, and Z are each |
| | selected (without replacement) from a SEQ ID NO: 5 AA position indicated in columns 1- |
| | 18. Expressed another way, the combinations of positions recited in the following |
| | combinations of columns: 1 + 2 + 3, 1 + 2 + 4, 1 + 2 + 5, 1 + 2 + 6, 1 + 2 + 7, 1 + 2 + 8, 1 + 2 + 9, 1 + 2 + 10, |
| | 1 + 2 + 11, 1 + 2 + 12, 1 + 2 + 13, 1 + 2 + 14, 1 + 2 + 15, 1 + 2 + 16, 1 + 2 + 17, 1 + 2 + 18, 1 + 3 + 4, 1 + 3 + 5, |
| | 1 + 3 + 6, 1 + 3 + 7, 1 + 3 + 8, 1 + 3 + 9, 1 + 3 + 10, 1 + 3 + 11, 1 + 3 + 12, 1 + 3 + 13, 1 + 3 + 14, 1 + 3 + 15, |
| | 1 + 3 + 16, 1 + 3 + 17, 1 + 3 + 18, 1 + 4 + 5, 1 + 4 + 6, 1 + 4 + 7, 1 + 4 + 8, 1 + 4 + 9, 1 + 4 + 10, 1 + 4 + 11, 1 + 4 + 12, |
| | 1 + 4 + 13, 1 + 4 + 14, 1 + 4 + 15, 1 + 4 + 16, 1 + 4 + 17, 1 + 4 + 18, 1 + 5 + 6, 1 + 5 + 7, 1 + 5 + 8, 1 + 5 + 9, |
| | 1 + 5 + 10, 1 + 5 + 11, 1 + 5 + 12, 1 + 5 + 13, 1 + 5 + 14, 1 + 5 + 15, 1 + 5 + 16, 1 + 5 + 17, 1 + 5 + 18, 1 + 6 + 7, |
| | 1 + 6 + 8, 1 + 6 + 9, 1 + 6 + 10, 1 + 6 + 11, 1 + 6 + 12, 1 + 6 + 13, 1 + 6 + 14, 1 + 6 + 15, 1 + 6 + 16, 1 + 6 + 17, |
| | 1 + 6 + 18, 1 + 7 + 8, 1 + 7 + 9, 1 + 7 + 10, 1 + 7 + 11, 1 + 7 + 12, 1 + 7 + 13, 1 + 7 + 14, 1 + 7 + 15, 1 + 7 + 16, |
| | 1 + 7 + 17, 1 + 7 + 18, 1 + 8 + 9, 1 + 8 + 10, 1 + 8 + 11, 1 + 8 + 12, 1 + 8 + 13, 1 + 8 + 14, 1 + 8 + 15, 1 + 8 + 16, |
| | 1 + 8 + 17, 1 + 8 + 18, 1 + 9 + 10, 1 + 9 + 11, 1 + 9 + 12, 1 + 9 + 13, 1 + 9 + 14, 1 + 9 + 15, 1 + 9 + 16, 1 + 9 + 17, |
| | 1 + 9 + 18, 1 + 10 + 11, 1 + 10 + 12, 1 + 10 + 13, 1 + 10 + 14, 1 + 10 + 15, 1 + 10 + 16, 1 + 10 + 17, 1 + 10 + 18, |
| | 1 + 11 + 12, 1 + 11 + 13, 1 + 11 + 14, 1 + 11 + 15, 1 + 11 + 16, 1 + 11 + 17, 1 + 11 + 18, 1 + 12 + 13, 1 + 12 + 14, |
| | 1 + 12 + 15, 1 + 12 + 16, 1 + 12 + 17, 1 + 12 + 18, 1 + 13 + 14, 1 + 13 + 15, 1 + 13 + 16, 1 + 13 + 17, 1 + 13 + 18, |
| | 1 + 14 + 15, 1 + 14 + 16, 1 + 14 + 17, 1 + 14 + 18, 1 + 15 + 16, 1 + 15 + 17, 1 + 15 + 18, 1 + 16 + 17, 1 + 16 + 18, |
| | 1 + 17 + 18, 2 + 3 + 4, 2 + 3 + 5, 2 + 3 + 6, 2 + 3 + 7, 2 + 3 + 8, 2 + 3 + 9, 2 + 3 + 10, 2 + 3 + 11, 2 + 3 + 12, 2 + 3 + 13, |
| | 2 + 3 + 14, 2 + 3 + 15, 2 + 3 + 16, 2 + 3 + 17, 2 + 3 + 18, 2 + 4 + 5, 2 + 4 + 6, 2 + 4 + 7, 2 + 4 + 8, 2 + 4 + 9, 2 + 4 + 10, |
| | 2 + 4 + 11, 2 + 4 + 12, 2 + 4 + 13, 2 + 4 + 14, 2 + 4 + 15, 2 + 4 + 16, 2 + 4 + 17, 2 + 4 + 18, 2 + 5 + 6, 2 + 5 + 7, |
| | 2 + 5 + 8, 2 + 5 + 9, 2 + 5 + 10, 2 + 5 + 11, 2 + 5 + 12, 2 + 5 + 13, 2 + 5 + 14, 2 + 5 + 15, 2 + 5 + 16, 2 + 5 + 17, |
| | 2 + 5 + 18, 2 + 6 + 7, 2 + 6 + 8, 2 + 6 + 9, 2 + 6 + 10, 2 + 6 + 11, 2 + 6 + 12, 2 + 6 + 13, 2 + 6 + 14, 2 + 6 + 15, |
| | 2 + 6 + 16, 2 + 6 + 17, 2 + 6 + 18, 2 + 7 + 8, 2 + 7 + 9, 2 + 7 + 10, 2 + 7 + 11, 2 + 7 + 12, 2 + 7 + 13, 2 + 7 + 14, |
| | 2 + 7 + 15, 2 + 7 + 16, 2 + 7 + 17, 2 + 7 + 18, 2 + 8 + 9, 2 + 8 + 10, 2 + 8 + 11, 2 + 8 + 12, 2 + 8 + 13, 2 + 8 + 14, |
| | 2 + 8 + 15, 2 + 8 + 16, 2 + 8 + 17, 2 + 8 + 18, 2 + 9 + 10, 2 + 9 + 11, 2 + 9 + 12, 2 + 9 + 13, 2 + 9 + 14, 2 + 9 + 15, |
| | 2 + 9 + 16, 2 + 9 + 17, 2 + 9 + 18, 2 + 10 + 11, 2 + 10 + 12, 2 + 10 + 13, 2 + 10 + 14, 2 + 10 + 15, 2 + 10 + 16, |
| | 2 + 10 + 17, 2 + 10 + 18, 2 + 11 + 12, 2 + 11 + 13, 2 + 11 + 14, 2 + 11 + 15, 2 + 11 + 16, 2 + 11 + 17, 2 + 11 + 18, |
| | 2 + 12 + 13, 2 + 12 + 14, 2 + 12 + 15, 2 + 12 + 16, 2 + 12 + 17, 2 + 12 + 18, 2 + 13 + 14, 2 + 13 + 15, 2 + 13 + 16, |
| | 2 + 13 + 17, 2 + 13 + 18, 2 + 14 + 15, 2 + 14 + 16, 2 + 14 + 17, 2 + 14 + 18, 2 + 15 + 16, 2 + 15 + 17, 2 + 15 + 18, |
| | 2 + 16 + 17, 2 + 16 + 18, 2 + 17 + 18, 3 + 4 + 5, 3 + 4 + 6, 3 + 4 + 7, 3 + 4 + 8, 3 + 4 + 9, 3 + 4 + 10, 3 + 4 + 11, |
| | 3 + 4 + 12, 3 + 4 + 13, 3 + 4 + 14, 3 + 4 + 15, 3 + 4 + 16, 3 + 4 + 17, 3 + 4 + 18, 3 + 5 + 6, 3 + 5 + 7, 3 + 5 + 8, |
| | 3 + 5 + 9, 3 + 5 + 10, 3 + 5 + 11, 3 + 5 + 12, 3 + 5 + 13, 3 + 5 + 14, 3 + 5 + 15, 3 + 5 + 16, 3 + 5 + 17, 3 + 5 + 18, |
| | 3 + 6 + 7, 3 + 6 + 8, 3 + 6 + 9, 3 + 6 + 10, 3 + 6 + 11, 3 + 6 + 12, 3 + 6 + 13, 3 + 6 + 14, 3 + 6 + 15, 3 + 6 + 16, |
| | 3 + 6 + 17, 3 + 6 + 18, 3 + 7 + 8, 3 + 7 + 9, 3 + 7 + 10, 3 + 7 + 11, 3 + 7 + 12, 3 + 7 + 13, 3 + 7 + 14, 3 + 7 + 15, |
| | 3 + 7 + 16, 3 + 7 + 17, 3 + 7 + 18, 3 + 8 + 9, 3 + 8 + 10, 3 + 8 + 11, 3 + 8 + 12, 3 + 8 + 13, 3 + 8 + 14, 3 + 8 + 15, |
| | 3 + 8 + 16, 3 + 8 + 17, 3 + 8 + 18, 3 + 9 + 10, 3 + 9 + 11, 3 + 9 + 12, 3 + 9 + 13, 3 + 9 + 14, 3 + 9 + 15, 3 + 9 + 16, |
| | 3 + 9 + 17, 3 + 9 + 18, 3 + 10 + 11, 3 + 10 + 12, 3 + 10 + 13, 3 + 10 + 14, 3 + 10 + 15, 3 + 10 + 16, 3 + 10 + 17, |
| | 3 + 10 + 18, 3 + 11 + 12, 3 + 11 + 13, 3 + 11 + 14, 3 + 11 + 15, 3 + 11 + 16, 3 + 11 + 17, 3 + 11 + 18, 3 + 12 + 13, |
| | 3 + 12 + 14, 3 + 12 + 15, 3 + 12 + 16, 3 + 12 + 17, 3 + 12 + 18, 3 + 13 + 14, 3 + 13 + 15, 3 + 13 + 16, 3 + 13 + 17, |
| | 3 + 13 + 18, 3 + 14 + 15, 3 + 14 + 16, 3 + 14 + 17, 3 + 14 + 18, 3 + 15 + 16, 3 + 15 + 17, 3 + 15 + 18, 3 + 16 + 17, |
| | 3 + 16 + 18, 3 + 17 + 18, 4 + 5 + 6, 4 + 5 + 7, 4 + 5 + 8, 4 + 5 + 9, 4 + 5 + 10, 4 + 5 + 11, 4 + 5 + 12, 4 + 5 + 13, |
| | 4 + 5 + 14, 4 + 5 + 15, 4 + 5 + 16, 4 + 5 + 17, 4 + 5 + 18, 4 + 6 + 7, 4 + 6 + 8, 4 + 6 + 9, 4 + 6 + 10, 4 + 6 + 11, |
| | 4 + 6 + 12, 4 + 6 + 13, 4 + 6 + 14, 4 + 6 + 15, 4 + 6 + 16, 4 + 6 + 17, 4 + 6 + 18, 4 + 7 + 8, 4 + 7 + 9, 4 + 7 + 10, |
| | 4 + 7 + 11, 4 + 7 + 12, 4 + 7 + 13, 4 + 7 + 14, 4 + 7 + 15, 4 + 7 + 16, 4 + 7 + 17, 4 + 7 + 18, 4 + 8 + 9, 4 + 8 + 10, |
| | 4 + 8 + 11, 4 + 8 + 12, 4 + 8 + 13, 4 + 8 + 14, 4 + 8 + 15, 4 + 8 + 16, 4 + 8 + 17, 4 + 8 + 18, 4 + 9 + 10, 4 + 9 + 11, |
| | 4 + 9 + 12, 4 + 9 + 13, 4 + 9 + 14, 4 + 9 + 15, 4 + 9 + 16, 4 + 9 + 17, 4 + 9 + 18, 4 + 10 + 11, 4 + 10 + 12, |
| | 4 + 10 + 13, 4 + 10 + 14, 4 + 10 + 15, 4 + 10 + 16, 4 + 10 + 17, 4 + 10 + 18, 4 + 11 + 12, 4 + 11 + 13, 4 + 11 + 14, |
| | 4 + 11 + 15, 4 + 11 + 16, 4 + 11 + 17, 4 + 11 + 18, 4 + 12 + 13, 4 + 12 + 14, 4 + 12 + 15, 4 + 12 + 16, 4 + 12 + 17, |
| | 4 + 12 + 18, 4 + 13 + 14, 4 + 13 + 15, 4 + 13 + 16, 4 + 13 + 17, 4 + 13 + 18, 4 + 14 + 15, 4 + 14 + 16, 4 + 14 + 17, |
| | 4 + 14 + 18, 4 + 15 + 16, 4 + 15 + 17, 4 + 15 + 18, 4 + 16 + 17, 4 + 16 + 18, 4 + 17 + 18, 5 + 6 + 7, 5 + 6 + 8, |
| | 5 + 6 + 9, 5 + 6 + 10, 5 + 6 + 11, 5 + 6 + 12, 5 + 6 + 13, 5 + 6 + 14, 5 + 6 + 15, 5 + 6 + 16, 5 + 6 + 17, 5 + 6 + 18, |
| | 5 + 7 + 8, 5 + 7 + 9, 5 + 7 + 10, 5 + 7 + 11, 5 + 7 + 12, 5 + 7 + 13, 5 + 7 + 14, 5 + 7 + 15, 5 + 7 + 16, 5 + 7 + 17, |
| | 5 + 7 + 18, 5 + 8 + 9, 5 + 8 + 10, 5 + 8 + 11, 5 + 8 + 12, 5 + 8 + 13, 5 + 8 + 14, 5 + 8 + 15, 5 + 8 + 16, 5 + 8 + 17, |
| | 5 + 8 + 18, 5 + 9 + 10, 5 + 9 + 11, 5 + 9 + 12, 5 + 9 + 13, 5 + 9 + 14, 5 + 9 + 15, 5 + 9 + 16, 5 + 9 + 17, 5 + 9 + 18, |
| | 5 + 10 + 11, 5 + 10 + 12, 5 + 10 + 13, 5 + 10 + 14, 5 + 10 + 15, 5 + 10 + 16, 5 + 10 + 17, 5 + 10 + 18, 5 + 11 + 12, |
| | 5 + 11 + 13, 5 + 11 + 14, 5 + 11 + 15, 5 + 11 + 16, 5 + 11 + 17, 5 + 11 + 18, 5 + 12 + 13, 5 + 12 + 14, 5 + 12 + 15, |
| | 5 + 12 + 16, 5 + 12 + 17, 5 + 12 + 18, 5 + 13 + 14, 5 + 13 + 15, 5 + 13 + 16, 5 + 13 + 17, 5 + 13 + 18, 5 + 14 + 15, |
| | 5 + 14 + 16, 5 + 14 + 17, 5 + 14 + 18, 5 + 15 + 16, 5 + 15 + 17, 5 + 15 + 18, 5 + 16 + 17, 5 + 16 + 18, 5 + 17 + 18, |
| | 6 + 7 + 8, 6 + 7 + 9, 6 + 7 + 10, 6 + 7 + 11, 6 + 7 + 12, 6 + 7 + 13, 6 + 7 + 14, 6 + 7 + 15, 6 + 7 + 16, 6 + 7 + 17, |
| | 6 + 7 + 18, 6 + 8 + 9, 6 + 8 + 10, 6 + 8 + 11, 6 + 8 + 12, 6 + 8 + 13, 6 + 8 + 14, 6 + 8 + 15, 6 + 8 + 16, 6 + 8 + 17, |
| | 6 + 8 + 18, 6 + 9 + 10, 6 + 9 + 11, 6 + 9 + 12, 6 + 9 + 13, 6 + 9 + 14, 6 + 9 + 15, 6 + 9 + 16, 6 + 9 + 17, 6 + 9 + 18, |
| | 6 + 10 + 11, 6 + 10 + 12, 6 + 10 + 13, 6 + 10 + 14, 6 + 10 + 15, 6 + 10 + 16, 6 + 10 + 17, 6 + 10 + 18, 6 + 11 + 12, |
| | 6 + 11 + 13, 6 + 11 + 14, 6 + 11 + 15, 6 + 11 + 16, 6 + 11 + 17, 6 + 11 + 18, 6 + 12 + 13, 6 + 12 + 14, 6 + 12 + 15, |
| | 6 + 12 + 16, 6 + 12 + 17, 6 + 12 + 18, 6 + 13 + 14, 6 + 13 + 15, 6 + 13 + 16, 6 + 13 + 17, 6 + 13 + 18, 6 + 14 + 15, |
| | 6 + 14 + 16, 6 + 14 + 17, 6 + 14 + 18, 6 + 15 + 16, 6 + 15 + 17, 6 + 15 + 18, 6 + 16 + 17, 6 + 16 + 18, 6 + 17 + 18, |
| | 7 + 8 + 9, 7 + 8 + 10, 7 + 8 + 11, 7 + 8 + 12, 7 + 8 + 13, 7 + 8 + 14, 7 + 8 + 15, 7 + 8 + 16, 7 + 8 + 17, 7 + 8 + 18, |
| | 7 + 9 + 10, 7 + 9 + 11, 7 + 9 + 12, 7 + 9 + 13, 7 + 9 + 14, 7 + 9 + 15, 7 + 9 + 16, 7 + 9 + 17, 7 + 9 + 18, 7 + 10 + 11, |
| | 7 + 10 + 12, 7 + 10 + 13, 7 + 10 + 14, 7 + 10 + 15, 7 + 10 + 16, 7 + 10 + 17, 7 + 10 + 18, 7 + 11 + 12, 7 + 11 + 13, |
| | 7 + 11 + 14, 7 + 11 + 15, 7 + 11 + 16, 7 + 11 + 17, 7 + 11 + 18, 7 + 12 + 13, 7 + 12 + 14, 7 + 12 + 15, 7 + 12 + 16, |
| | 7 + 12 + 17, 7 + 12 + 18, 7 + 13 + 14, 7 + 13 + 15, 7 + 13 + 16, 7 + 13 + 17, 7 + 13 + 18, 7 + 14 + 15, 7 + 14 + 16, |
| | 7 + 14 + 17, 7 + 14 + 18, 7 + 15 + 16, 7 + 15 + 17, 7 + 15 + 18, 7 + 16 + 17, 7 + 16 + 18, 7 + 17 + 18, 8 + 9 + 10, |
| | 8 + 9 + 11, 8 + 9 + 12, 8 + 9 + 13, 8 + 9 + 14, 8 + 9 + 15, 8 + 9 + 16, 8 + 9 + 17, 8 + 9 + 18, 8 + 10 + 11, 8 + 10 + 12, |
| | 8 + 10 + 13, 8 + 10 + 14, 8 + 10 + 15, 8 + 10 + 16, 8 + 10 + 17, 8 + 10 + 18, 8 + 11 + 12, 8 + 11 + 13, 8 + 11 + 14, |
| | 8 + 11 + 15, 8 + 11 + 16, 8 + 11 + 17, 8 + 11 + 18, 8 + 12 + 13, 8 + 12 + 14, 8 + 12 + 15, 8 + 12 + 16, 8 + 12 + 17, |
| | 8 + 12 + 18, 8 + 13 + 14, 8 + 13 + 15, 8 + 13 + 16, 8 + 13 + 17, 8 + 13 + 18, 8 + 14 + 15, 8 + 14 + 16, 8 + 14 + 17, |
| | 8 + 14 + 18, 8 + 15 + 16, 8 + 15 + 17, 8 + 15 + 18, 8 + 16 + 17, 8 + 16 + 18, 8 + 17 + 18, 9 + 10 + 11, 9 + 10 + 12, |
| | 9 + 10 + 13, 9 + 10 + 14, 9 + 10 + 15, 9 + 10 + 16, 9 + 10 + 17, 9 + 10 + 18, 9 + 11 + 12, 9 + 11 + 13, 9 + 11 + 14, |
| | 9 + 11 + 15, 9 + 11 + 16, 9 + 11 + 17, 9 + 11 + 18, 9 + 12 + 13, 9 + 12 + 14, 9 + 12 + 15, 9 + 12 + 16, 9 + 12 + 17, |
| | 9 + 12 + 18, 9 + 13 + 14, 9 + 13 + 15, 9 + 13 + 16, 9 + 13 + 17, 9 + 13 + 18, 9 + 14 + 15, 9 + 14 + 16, 9 + 14 + 17, |
| | 9 + 14 + 18, 9 + 15 + 16, 9 + 15 + 17, 9 + 15 + 18, 9 + 16 + 17, 9 + 16 + 18, 9 + 17 + 18, 10 + 11 + 12, |
| | 10 + 11 + 13, 10 + 11 + 14, 10 + 11 + 15, 10 + 11 + 16, 10 + 11 + 17, 10 + 11 + 18, 10 + 12 + 13, 10 + 12 + 14, |
| | 10 + 12 + 15, 10 + 12 + 16, 10 + 12 + 17, 10 + 12 + 18, 10 + 13 + 14, 10 + 13 + 15, 10 + 13 + 16, 10 + 13 + 17, |
| | 10 + 13 + 18, 10 + 14 + 15, 10 + 14 + 16, 10 + 14 + 17, 10 + 14 + 18, 10 + 15 + 16, 10 + 15 + 17, 10 + 15 + 18, |
| | 10 + 16 + 17, 10 + 16 + 18, 10 + 17 + 18, 11 + 12 + 13, 11 + 12 + 14, 11 + 12 + 15, 11 + 12 + 16, 11 + 12 + 17, |
| | 11 + 12 + 18. 11 + 13 + 14, 11 + 13 + 15, 11 + 13 + 16, 11 + 13 + 17, 11 + 13 + 18, 11 + 14 + 15, 11 + 14 + 16, |
| | 11 + 14 + 17, 11 + 14 + 18, 11 + 15 + 16, 11 + 15 + 17, 11 + 15 + 18, 11 + 16 + 17, 11 + 16 + 18, 11 + 17 + 18, |
| | 12 + 13 + 14, 12 + 13 + 15, 12 + 13 + 16, 12 + 13 + 17, 12 + 13 + 18, 12 + 14 + 15, 12 + 14 + 16, 12 + 14 + 17, |
| | 12 + 14 + 18, 12 + 15 + 16, 12 + 15 + 17, 12 + 15 + 18, 12 + 16 + 17, 12 + 16 + 18, 12 + 17 + 18, 13 + 14 + 15, |
| | 13 + 14 + 16, 13 + 14 + 17, 13 + 14 + 18, 13 + 15 + 16, 13 + 15 + 17, 13 + 15 + 18, 13 + 16 + 17, 13 + 16 + 18, |
| | 13 + 17 + 18, 14 + 15 + 16, 14 + 15 + 17, 14 + 15 + 18, 14 + 16 + 17, 14 + 16 + 18, 14 + 17 + 18, 15 + 16 + 17, |
| | 15 + 16 + 18, 15 + 17 + 18, and 16 + 17 + 18. |
| C | Each and every unique 4-position combination of X & Y & Z & α, wherein X, Y, and Z are |
| | together one of the 3-position combinations recited in “B” (above), and α is selected from a |
| | SEQ ID NO: 5 amino acid position recited in one of columns 1 to 18, without replacement |
| | (i.e. X ≠ Y ≠ Z ≠ α). |

In some examples, the first ABD includes an HCVD that is at least 90%, 95%, or 98% identical to SEQ ID NO: 4, where the HCVD includes a histidine at any of the specific combinations of two or more amino acid positions in SEQ ID NO: 4 listed in Table 1; and an LCVD that that is at least 90%, 95%, or 98% identical to SEQ ID NO: 5, where the LCVD includes a histidine at any of the specific combinations of two or more amino acid positions in SEQ ID NO: 5 listed in Table 2.

In some examples, the first ABD includes an LCVD that is at least 90%, 95%, or 98% identical to SEQ ID NO: 5, where the LCVD includes a histidine at any of the specific combinations of two or more amino acid positions in SEQ ID NO: 5 listed in Table 2, and/or where the LCVD includes an alanine in position 91 in SEQ ID NO: 5; and an HCVD that that is at least 90%, 95%, or 98% identical to SEQ ID NO: 4, where the HCVD includes a histidine at any of the specific combinations of two or more amino acid positions in SEQ ID NO: 4 listed in Table 1.

In some examples, the first ABD comprises an LCVD comprising SEQ ID NO: 5, and an HCVD that is at least 90%, 95%, or 98% identical to SEQ ID NO: 4, where the HCVD includes a histidine at any of the specific combinations of two or more amino acid positions in SEQ ID NO: 4 listed in Table 1.

In some examples, the first ABD comprises an LCVD that is at least 90%, 95%, or 98% identical to SEQ ID NO: 5, where the LCVD includes a histidine at position 28 in SEQ ID NO: 5; and an HCVD that is at least 90%, 95%, or 98% identical to SEQ ID NO: 4, where the HCVD includes a histidine at any of the specific combinations of two or more amino acid positions in SEQ ID NO: 4 listed in Table 1.

In some examples, the first ABD comprises an LCVD that is at least 90%, 95%, or 98% identical to SEQ ID NO: 5, where the LCVD includes a histidine at position 29 in SEQ ID NO: 5; and an HCVD that is at least 90%, 95%, or 98% identical to SEQ ID NO: 4, where the HCVD includes a histidine at any of the specific combinations of two or more amino acid positions in SEQ ID NO: 4 listed in Table 1.

In some examples, the first ABD comprises an LCVD that is at least 90%, 95%, or 98% identical to SEQ ID NO: 5, where the LCVD includes a histidine at position 30 in SEQ ID NO: 5; and an HCVD that is at least 90%, 95%, or 98% identical to SEQ ID NO: 4, where the HCVD includes a histidine at any of the specific combinations of two or more amino acid positions in SEQ ID NO: 4 listed in Table 1.

In some examples, the first ABD comprises an LCVD that is at least 90%, 95%, or 98% identical to SEQ ID NO: 5, where the LCVD includes an alanine at position 91 in SEQ ID NO: 5; and an HCVD that is at least 90%, 95%, or 98% identical to SEQ ID NO: 4, wherein the HCVD includes a histidine at any of the specific combinations of two or more amino acid positions in SEQ ID NO: 4 listed in Table 1.

In some examples, the first ABD comprises an LCVD that is at least 90%, 95%, or 98% identical to SEQ ID NO: 5, wherein the LCVD includes a histidine at position 31 in SEQ ID NO: 5; and an HCVD that is at least 90%, 95%, or 98% identical to SEQ ID NO: 4, wherein the HCVD includes a histidine at any of the specific combinations of two or more amino acid positions in SEQ ID NO: 4 listed in Table 1.

In some examples, the first ABD comprises an LCVD that is at least 90%, 95%, or 98% identical to SEQ ID NO: 5, wherein the LCVD includes a histidine at position 32 in SEQ ID NO: 5; and an HCVD that is at least 90%, 95%, or 98% identical to SEQ ID NO: 4, where the HCVD includes a histidine at any of the specific combinations of two or more amino acid positions in SEQ ID NO: 4 listed in Table 1.

In some examples, the first ABD comprises an LCVD that is at least 90%, 95%, or 98% identical to SEQ ID NO: 5, wherein the LCVD includes a histidine at position 33 in SEQ ID NO: 5; and an HCVD that is at least 90%, 95%, or 98% identical to SEQ ID NO: 4, where the HCVD includes a histidine at any of the specific combinations of two or more amino acid positions in SEQ ID NO: 4 listed in Table 1.

In some examples, the first ABD comprises an LCVD that is at least 90%, 95%, or 98% identical to SEQ ID NO: 5, wherein the LCVD includes a histidine at position 50 in SEQ ID NO: 5; and an HCVD that is at least 90%, 95%, or 98% identical to SEQ ID NO: 4, where the HCVD includes a histidine at any of the specific combinations of two or more amino acid positions in SEQ ID NO: 4 listed in Table 1.

In some examples, the first ABD comprises an LCVD that is at least 90%, 95%, or 98% identical to SEQ ID NO: 5, wherein the LCVD includes a histidine at position 51 in SEQ ID NO: 5; and an HCVD that is at least 90%, 95%, or 98% identical to SEQ ID NO: 4, where the HCVD includes a histidine at any of the specific combinations of two or more amino acid positions in SEQ ID NO: 4 listed in Table 1.

In some examples, the first ABD comprises an LCVD that is at least 90%, 95%, or 98% identical to SEQ ID NO: 5, wherein the LCVD includes a histidine at position 52 in SEQ ID NO: 5; and an HCVD that is at least 90%, 95%, or 98% identical to SEQ ID NO: 4, where the HCVD includes a histidine at any of the specific combinations of two or more amino acid positions in SEQ ID NO: 4 listed in Table 1.

In some examples, the first ABD comprises an LCVD that is at least 90%, 95%, or 98% identical to SEQ ID NO: 5, wherein the LCVD includes a histidine at position 53 in SEQ ID NO: 5; and an HCVD that is at least 90%, 95%, or 98% identical to SEQ ID NO: 4, where the HCVD includes a histidine at any of the specific combinations of two or more amino acid positions in SEQ ID NO: 4 listed in Table 1.

In some examples, the first ABD comprises an LCVD that is at least 90%, 95%, or 98% identical to SEQ ID NO: 5, wherein the LCVD includes a histidine at position 89 in SEQ ID NO: 5; and an HCVD that is at least 90%, 95%, or 98% identical to SEQ ID NO: 4, where the HCVD includes a histidine at any of the specific combinations of two or more amino acid positions in SEQ ID NO: 4 listed in Table 1.

In some examples, the first ABD comprises an LCVD that is at least 90%, 95%, or 98% identical to SEQ ID NO: 5, wherein the LCVD includes a histidine at position 92 in SEQ ID NO: 5; and an HCVD that is at least 90%, 95%, or 98% identical to SEQ ID NO: 4, where the HCVD includes a histidine at any of the specific combinations of two or more amino acid positions in SEQ ID NO: 4 listed in Table 1.

In some examples, the first ABD comprises an LCVD that is at least 90%, 95%, or 98% identical to SEQ ID NO: 5, wherein the LCVD includes a histidine at position 93 in SEQ ID NO: 5; and an HCVD that is at least 90%, 95%, or 98% identical to SEQ ID NO: 4, where the HCVD includes a histidine at any of the specific combinations of two or more amino acid positions in SEQ ID NO: 4 listed in Table 1.

In some examples, the first ABD comprises an LCVD that is at least 90%, 95%, or 98% identical to SEQ ID NO: 5, wherein the LCVD includes a histidine at position 94 in SEQ ID NO: 5; and an HCVD that is at least 90%, 95%, or 98% identical to SEQ ID NO: 4, where the HCVD includes a histidine at any of the specific combinations of two or more amino acid positions in SEQ ID NO: 4 listed in Table 1.

In some examples, the first ABD comprises an LCVD that is at least 90%, 95%, or 98% identical to SEQ ID NO: 5, wherein the LCVD includes a histidine at position 95 in SEQ ID NO: 5; and an HCVD that is at least 90%, 95%, or 98% identical to SEQ ID NO: 4, where the HCVD includes a histidine at any of the specific combinations of two or more amino acid positions in SEQ ID NO: 4 listed in Table 1.

In some examples, the first ABD comprises an LCVD that is at least 90%, 95%, or 98% identical to SEQ ID NO: 5, wherein the LCVD includes a histidine at position 96 in SEQ ID NO: 5; and an HCVD that is at least 90%, 95%, or 98% identical to SEQ ID NO: 4, where the HCVD includes a histidine at any of the specific combinations of two or more amino acid positions in SEQ ID NO: 4 listed in Table 1.

In some examples, the first ABD comprises an LCVD that is at least 90%, 95%, or 98% identical to SEQ ID NO: 5, wherein the LCVD includes a histidine at position 97 in SEQ ID NO: 5; and an HCVD that is at least 90%, 95%, or 98% identical to SEQ ID NO: 4, where the HCVD includes a histidine at any of the specific combinations of two or more amino acid positions in SEQ ID NO: 4 listed in Table 1.

In some examples, the first ABD comprises an HCVD that is at least 90%, 95%, or 98% identical to SEQ ID NO: 4, wherein the HCVD includes a histidine at any one of positions 29, 32, 33, 50, 52, 53, 60, 61, 62, 63, 64, 65, 66, 99, 101, 104, 105, and 108 in SEQ ID NO: 4; and an LCVD that is at least 90%, 95%, or 98% identical to SEQ ID NO: 5, where the LCVD includes a histidine at any of the specific combinations of two or more amino acid positions in SEQ ID NO: 5 listed in Table 2.

In some examples, the first ABD comprises an HCVD of SEQ ID NO: 4, one of the sequences set forth in SEQ ID NOs: 12-52, or one of the sequences set forth in SEQ ID NOs: 313-350; and/or the first ABD includes an LCVD of SEQ ID NO: 5, one of the sequences set forth in SEQ ID NOs: 53-79, or one of the sequences set forth in SEQ ID NOs: 351-396.

In some examples of the ABPCs, the first ABD includes an LCVD comprising the sequence of SEQ ID NO: 5, one of the sequences set forth in SEQ ID NOs: 53-79, or one of the sequences set forth in SEQ ID NOs: 351-396 and an HCVD comprising SEQ ID NO: 4, one of the sequences set forth in SEQ ID NOs: 12-52, or one of the sequences set forth in SEQ ID NOs: 313-350.

In some examples, the first ABD includes an HCVD comprising the sequence of SEQ ID NO: 4, and an LCVD comprising the sequence of SEQ ID NO: 5, one of the sequences set forth in SEQ ID NOs: 53-79, or one of the sequences set forth in SEQ ID NOs: 351-396.

In some examples, the first ABD includes an HCVD comprising the sequence of SEQ ID NO: 12, and an LCVD comprising the sequence of SEQ ID NO: 5, one of the sequences set forth in SEQ ID NOs: 53-79, or one of the sequences set forth in SEQ ID NOs: 351-396.

In some examples, the first ABD includes an HCVD comprising the sequence of SEQ ID NO: 13, and an LCVD comprising the sequence of SEQ ID NO: 5, one of the sequences set forth in SEQ ID NOs: 53-79, or one of the sequences set forth in SEQ ID NOs: 351-396.

In some examples, the first ABD includes an HCVD comprising the sequence of SEQ ID NO: 14, and an LCVD comprising the sequence of SEQ ID NO: 5, one of the sequences set forth in SEQ ID NOs: 53-79, or one of the sequences set forth in SEQ ID NOs: 351-396.

In some examples, the first ABD includes an HCVD comprising the sequence of SEQ ID NO: 15, and an LCVD comprising the sequence of SEQ ID NO: 5, one of the sequences set forth in SEQ ID NOs: 53-79, or one of the sequences set forth in SEQ ID NOs: 351-396.

In some examples, the first ABD includes an HCVD comprising the sequence of SEQ ID NO: 16, and an LCVD comprising the sequence of SEQ ID NO: 5, one of the sequences set forth in SEQ ID NOs: 53-79, or one of the sequences set forth in SEQ ID NOs: 351-396.

In some examples, the first ABD includes an HCVD comprising the sequence of SEQ ID NO: 17, and an LCVD comprising the sequence of SEQ ID NO: 5, one of the sequences set forth in SEQ ID NOs: 53-79, or one of the sequences set forth in SEQ ID NOs: 351-396.

In some examples, the first ABD includes an HCVD comprising the sequence of SEQ ID NO: 18, and an LCVD comprising the sequence of SEQ ID NO: 5, one of the sequences set forth in SEQ ID NOs: 53-79, or one of the sequences set forth in SEQ ID NOs: 351-396.

In some examples, the first ABD includes an HCVD comprising the sequence of SEQ ID NO: 19, and an LCVD comprising the sequence of SEQ ID NO: 5, one of the sequences set forth in SEQ ID NOs: 53-79, or one of the sequences set forth in SEQ ID NOs: 351-396.

In some examples, the first ABD includes an HCVD comprising the sequence of SEQ ID NO: 20, and an LCVD comprising the sequence of SEQ ID NO: 5, one of the sequences set forth in SEQ ID NOs: 53-79, or one of the sequences set forth in SEQ ID NOs: 351-396.

In some examples, the first ABD includes an HCVD comprising the sequence of SEQ ID NO: 21, and an LCVD comprising the sequence of SEQ ID NO: 5, one of the sequences set forth in SEQ ID NOs: 53-79, or one of the sequences set forth in SEQ ID NOs: 351-396.

In some examples, the first ABD includes an HCVD comprising the sequence of SEQ ID NO: 22, and an LCVD comprising the sequence of SEQ ID NO: 5, one of the sequences set forth in SEQ ID NOs: 53-79, or one of the sequences set forth in SEQ ID NOs: 351-396.

In some examples, the first ABD includes an HCVD comprising the sequence of SEQ ID NO: 23, and an LCVD comprising the sequence of SEQ ID NO: 5, one of the sequences set forth in SEQ ID NOs: 53-79, or one of the sequences set forth in SEQ ID NOs: 351-396.

In some examples, the first ABD includes an HCVD comprising the sequence of SEQ ID NO: 24, and an LCVD comprising the sequence of SEQ ID NO: 5, one of the sequences set forth in SEQ ID NOs: 53-79, or one of the sequences set forth in SEQ ID NOs: 351-396.

In some examples, the first ABD includes an HCVD comprising the sequence of SEQ ID NO: 25, and an LCVD comprising the sequence of SEQ ID NO: 5, one of the sequences set forth in SEQ ID NOs: 53-79, or one of the sequences set forth in SEQ ID NOs: 351-396.

In some examples, the first ABD includes an HCVD comprising the sequence of SEQ ID NO: 26, and an LCVD comprising the sequence of SEQ ID NO: 5, one of the sequences set forth in SEQ ID NOs: 53-79, or one of the sequences set forth in SEQ ID NOs: 351-396.

In some examples, the first ABD includes an HCVD comprising the sequence of SEQ ID NO: 27, and an LCVD comprising the sequence of SEQ ID NO: 5, one of the sequences set forth in SEQ ID NOs: 53-79, or one of the sequences set forth in SEQ ID NOs: 351-396.

In some examples, the first ABD includes an HCVD comprising the sequence of SEQ ID NO: 28, and an LCVD comprising the sequence of SEQ ID NO: 5, one of the sequences set forth in SEQ ID NOs: 53-79, or one of the sequences set forth in SEQ ID NOs: 351-396.

In some examples, the first ABD includes an HCVD comprising the sequence of SEQ ID NO: 29, and an LCVD comprising the sequence of SEQ ID NO: 5, one of the sequences set forth in SEQ ID NOs: 53-79, or one of the sequences set forth in SEQ ID NOs: 351-396.

In some examples, the first ABD includes an HCVD comprising the sequence of SEQ ID NO: 30, and an LCVD comprising the sequence of SEQ ID NO: 5, one of the sequences set forth in SEQ ID NOs: 53-79, or one of the sequences set forth in SEQ ID NOs: 351-396.

In some examples, the first ABD includes an HCVD comprising the sequence of SEQ ID NO: 31, and an LCVD comprising the sequence of SEQ ID NO: 5, one of the sequences set forth in SEQ ID NOs: 53-79, or one of the sequences set forth in SEQ ID NOs: 351-396.

In some examples, the first ABD includes an HCVD comprising the sequence of SEQ ID NO: 32, and an LCVD comprising the sequence of SEQ ID NO: 5, one of the sequences set forth in SEQ ID NOs: 53-79, or one of the sequences set forth in SEQ ID NOs: 351-396.

In some examples, the first ABD includes an HCVD comprising the sequence of SEQ ID NO: 33, and an LCVD comprising the sequence of SEQ ID NO: 5, one of the sequences set forth in SEQ ID NOs: 53-79, or one of the sequences set forth in SEQ ID NOs: 351-396.

In some examples, the first ABD includes an HCVD comprising the sequence of SEQ ID NO: 34, and an LCVD comprising the sequence of SEQ ID NO: 5, one of the sequences set forth in SEQ ID NOs: 53-79, or one of the sequences set forth in SEQ ID NOs: 351-396.

In some examples, the first ABD includes an HCVD comprising the sequence of SEQ ID NO: 35, and an LCVD comprising the sequence of SEQ ID NO: 5, one of the sequences set forth in SEQ ID NOs: 53-79, or one of the sequences set forth in SEQ ID NOs: 351-396.

In some examples, the first ABD includes an HCVD comprising the sequence of SEQ ID NO: 36, and an LCVD comprising the sequence of SEQ ID NO: 5, one of the sequences set forth in SEQ ID NOs: 53-79, or one of the sequences set forth in SEQ ID NOs: 351-396.

In some examples, the first ABD includes an HCVD comprising the sequence of SEQ ID NO: 37, and an LCVD comprising the sequence of SEQ ID NO: 5, one of the sequences set forth in SEQ ID NOs: 53-79, or one of the sequences set forth in SEQ ID NOs: 351-396.

In some examples, the first ABD includes an HCVD comprising the sequence of SEQ ID NO: 38, and an LCVD comprising the sequence of SEQ ID NO: 5, one of the sequences set forth in SEQ ID NOs: 53-79, or one of the sequences set forth in SEQ ID NOs: 351-396.

In some examples, the first ABD includes an HCVD comprising the sequence of SEQ ID NO: 39, and an LCVD comprising the sequence of SEQ ID NO: 5, one of the sequences set forth in SEQ ID NOs: 53-79, or one of the sequences set forth in SEQ ID NOs: 351-396.

In some examples, the first ABD includes an HCVD comprising the sequence of SEQ ID NO: 40, and an LCVD comprising the sequence of SEQ ID NO: 5, one of the sequences set forth in SEQ ID NOs: 53-79, or one of the sequences set forth in SEQ ID NOs: 351-396.

In some examples, the first ABD includes an HCVD comprising the sequence of SEQ ID NO: 41, and an LCVD comprising the sequence of SEQ ID NO: 5, one of the sequences set forth in SEQ ID NOs: 53-79, or one of the sequences set forth in SEQ ID NOs: 351-396.

In some examples, the first ABD includes an HCVD comprising the sequence of SEQ ID NO: 42, and an LCVD comprising the sequence of SEQ ID NO: 5, one of the sequences set forth in SEQ ID NOs: 53-79, or one of the sequences set forth in SEQ ID NOs: 351-396.

In some examples, the first ABD includes an HCVD comprising the sequence of SEQ ID NO: 43, and an LCVD comprising the sequence of SEQ ID NO: 5, one of the sequences set forth in SEQ ID NOs: 53-79, or one of the sequences set forth in SEQ ID NOs: 351-396.

In some examples, the first ABD includes an HCVD comprising the sequence of SEQ ID NO: 44, and an LCVD comprising the sequence of SEQ ID NO: 5, one of the sequences set forth in SEQ ID NOs: 53-79, or one of the sequences set forth in SEQ ID NOs: 351-396.

In some examples, the first ABD includes an HCVD comprising the sequence of SEQ ID NO: 45, and an LCVD comprising the sequence of SEQ ID NO: 5, one of the sequences set forth in SEQ ID NOs: 53-79, or one of the sequences set forth in SEQ ID NOs: 351-396.

In some examples, the first ABD includes an HCVD comprising the sequence of SEQ ID NO: 46, and an LCVD comprising the sequence of SEQ ID NO: 5, one of the sequences set forth in SEQ ID NOs: 53-79, or one of the sequences set forth in SEQ ID NOs: 351-396.

In some examples, the first ABD includes an HCVD comprising the sequence of SEQ ID NO: 47, and an LCVD comprising the sequence of SEQ ID NO: 5, one of the sequences set forth in SEQ ID NOs: 53-79, or one of the sequences set forth in SEQ ID NOs: 351-396.

In some examples, the first ABD includes an HCVD comprising the sequence of SEQ ID NO: 48, and an LCVD comprising the sequence of SEQ ID NO: 5, one of the sequences set forth in SEQ ID NOs: 53-79, or one of the sequences set forth in SEQ ID NOs: 351-396.

In some examples, the first ABD includes an HCVD comprising the sequence of SEQ ID NO: 49, and an LCVD comprising the sequence of SEQ ID NO: 5, one of the sequences set forth in SEQ ID NOs: 53-79, or one of the sequences set forth in SEQ ID NOs: 351-396.

In some examples, the first ABD includes an HCVD comprising the sequence of SEQ ID NO: 50, and an LCVD comprising the sequence of SEQ ID NO: 5, one of the sequences set forth in SEQ ID NOs: 53-79, or one of the sequences set forth in SEQ ID NOs: 351-396.

In some examples, the first ABD includes an HCVD comprising the sequence of SEQ ID NO: 51, and an LCVD comprising the sequence of SEQ ID NO: 5, one of the sequences set forth in SEQ ID NOs: 53-79, or one of the sequences set forth in SEQ ID NOs: 351-396.

In some examples, the first ABD includes an HCVD comprising the sequence of SEQ ID NO: 52, and an LCVD comprising the sequence of SEQ ID NO: 5, one of the sequences set forth in SEQ ID NOs: 53-79, or one of the sequences set forth in SEQ ID NOs: 351-396.

In some examples, the first ABD includes an HCVD comprising the sequence of SEQ ID NO: 313, and an LCVD comprising the sequence of SEQ ID NO: 5, one of the sequences as set forth in SEQ ID NOs: 53-79, or one of the sequences set forth in SEQ ID NOs: 351-396.

In some examples, the first ABD includes an HCVD comprising the sequence of SEQ ID NO: 314, and an LCVD comprising the sequence of SEQ ID NO: 5, one of the sequences as set forth in SEQ ID NOs: 53-79, or one of the sequences set forth in SEQ ID NOs: 351-396.

In some examples, the first ABD includes an HCVD comprising the sequence of SEQ ID NO: 315, and an LCVD comprising the sequence of SEQ ID NO: 5, one of the sequences as set forth in SEQ ID NOs: 53-79, or one of the sequences set forth in SEQ ID NOs: 351-396.

In some examples, the first ABD includes an HCVD comprising the sequence of SEQ ID NO: 316, and an LCVD comprising the sequence of SEQ ID NO: 5, one of the sequences as set forth in SEQ ID NOs: 53-79, or one of the sequences set forth in SEQ ID NOs: 351-396.

In some examples, the first ABD includes an HCVD comprising the sequence of SEQ ID NO: 317, and an LCVD comprising the sequence of SEQ ID NO: 5, one of the sequences as set forth in SEQ ID NOs: 53-79, or one of the sequences set forth in SEQ ID NOs: 351-396.

In some examples, the first ABD includes an HCVD comprising the sequence of SEQ ID NO: 318, and an LCVD comprising the sequence of SEQ ID NO: 5, one of the sequences as set forth in SEQ ID NOs: 53-79, or one of the sequences set forth in SEQ ID NOs: 351-396.

In some examples, the first ABD includes an HCVD comprising the sequence of SEQ ID NO: 319, and an LCVD comprising the sequence of SEQ ID NO: 5, one of the sequences as set forth in SEQ ID NOs: 53-79, or one of the sequences set forth in SEQ ID NOs: 351-396.

In some examples, the first ABD includes an HCVD comprising the sequence of SEQ ID NO: 320, and an LCVD comprising the sequence of SEQ ID NO: 5, one of the sequences as set forth in SEQ ID NOs: 53-79, or one of the sequences set forth in SEQ ID NOs: 351-396.

In some examples, the first ABD includes an HCVD comprising the sequence of SEQ ID NO: 321, and an LCVD comprising the sequence of SEQ ID NO: 5, one of the sequences as set forth in SEQ ID NOs: 53-79, or one of the sequences set forth in SEQ ID NOs: 351-396.

In some examples, the first ABD includes an HCVD comprising the sequence of SEQ ID NO: 322, and an LCVD comprising the sequence of SEQ ID NO: 5, one of the sequences as set forth in SEQ ID NOs: 53-79, or one of the sequences set forth in SEQ ID NOs: 351-396.

In some examples, the first ABD includes an HCVD comprising the sequence of SEQ ID NO: 323, and an LCVD comprising the sequence of SEQ ID NO: 5, one of the sequences as set forth in SEQ ID NOs: 53-79, or one of the sequences set forth in SEQ ID NOs: 351-396.

In some examples, the first ABD includes an HCVD comprising the sequence of SEQ ID NO: 324, and an LCVD comprising the sequence of SEQ ID NO: 5, one of the sequences as set forth in SEQ ID NOs: 53-79, or one of the sequences set forth in SEQ ID NOs: 351-396.

In some examples, the first ABD includes an HCVD comprising the sequence of SEQ ID NO: 325, and an LCVD comprising the sequence of SEQ ID NO: 5, one of the sequences as set forth in SEQ ID NOs: 53-79, or one of the sequences set forth in SEQ ID NOs: 351-396.

In some examples, the first ABD includes an HCVD comprising the sequence of SEQ ID NO: 326, and an LCVD comprising the sequence of SEQ ID NO: 5, one of the sequences as set forth in SEQ ID NOs: 53-79, or one of the sequences set forth in SEQ ID NOs: 351-396.

In some examples, the first ABD includes an HCVD comprising the sequence of SEQ ID NO: 327, and an LCVD comprising the sequence of SEQ ID NO: 5, one of the sequences as set forth in SEQ ID NOs: 53-79, or one of the sequences set forth in SEQ ID NOs: 351-396.

In some examples, the first ABD includes an HCVD comprising the sequence of SEQ ID NO: 328, and an LCVD comprising the sequence of SEQ ID NO: 5, one of the sequences as set forth in SEQ ID NOs: 53-79, or one of the sequences set forth in SEQ ID NOs: 351-396.

In some examples, the first ABD includes an HCVD comprising the sequence of SEQ ID NO: 329, and an LCVD comprising the sequence of SEQ ID NO: 5, one of the sequences as set forth in SEQ ID NOs: 53-79, or one of the sequences set forth in SEQ ID NOs: 351-396.

In some examples, the first ABD includes an HCVD comprising the sequence of SEQ ID NO: 330, and an LCVD comprising the sequence of SEQ ID NO: 5, one of the sequences as set forth in SEQ ID NOs: 53-79, or one of the sequences set forth in SEQ ID NOs: 351-396.

In some examples, the first ABD includes an HCVD comprising the sequence of SEQ ID NO: 331, and an LCVD comprising the sequence of SEQ ID NO: 5, one of the sequences as set forth in SEQ ID NOs: 53-79, or one of the sequences set forth in SEQ ID NOs: 351-396.

In some examples, the first ABD includes an HCVD comprising the sequence of SEQ ID NO: 332, and an LCVD comprising the sequence of SEQ ID NO: 5, one of the sequences as set forth in SEQ ID NOs: 53-79, or one of the sequences set forth in SEQ ID NOs: 351-396.

In some examples, the first ABD includes an HCVD comprising the sequence of SEQ ID NO: 333, and an LCVD comprising the sequence of SEQ ID NO: 5, one of the sequences as set forth in SEQ ID NOs: 53-79, or one of the sequences set forth in SEQ ID NOs: 351-396.

In some examples, the first ABD includes an HCVD comprising the sequence of SEQ ID NO: 334, and an LCVD comprising the sequence of SEQ ID NO: 5, one of the sequences as set forth in SEQ ID NOs: 53-79, or one of the sequences set forth in SEQ ID NOs: 351-396.

In some examples, the first ABD includes an HCVD comprising the sequence of SEQ ID NO: 335, and an LCVD comprising the sequence of SEQ ID NO: 5, one of the sequences as set forth in SEQ ID NOs: 53-79, or one of the sequences set forth in SEQ ID NOs: 351-396.

In some examples, the first ABD includes an HCVD comprising the sequence of SEQ ID NO: 336, and an LCVD comprising the sequence of SEQ ID NO: 5, one of the sequences as set forth in SEQ ID NOs: 53-79, or one of the sequences set forth in SEQ ID NOs: 351-396.

In some examples, the first ABD includes an HCVD comprising the sequence of SEQ ID NO: 337, and an LCVD comprising the sequence of SEQ ID NO: 5, one of the sequences as set forth in SEQ ID NOs: 53-79, or one of the sequences set forth in SEQ ID NOs: 351-396.

In some examples, the first ABD includes an HCVD comprising the sequence of SEQ ID NO: 338, and an LCVD comprising the sequence of SEQ ID NO: 5, one of the sequences as set forth in SEQ ID NOs: 53-79, or one of the sequences set forth in SEQ ID NOs: 351-396.

In some examples, the first ABD includes an HCVD comprising the sequence of SEQ ID NO: 339, and an LCVD comprising the sequence of SEQ ID NO: 5, one of the sequences as set forth in SEQ ID NOs: 53-79, or one of the sequences set forth in SEQ ID NOs: 351-396.

In some examples, the first ABD includes an HCVD comprising the sequence of SEQ ID NO: 340, and an LCVD comprising the sequence of SEQ ID NO: 5, one of the sequences as set forth in SEQ ID NOs: 53-79, or one of the sequences set forth in SEQ ID NOs: 351-396.

In some examples, the first ABD includes an HCVD comprising the sequence of SEQ ID NO: 341, and an LCVD comprising the sequence of SEQ ID NO: 5, one of the sequences as set forth in SEQ ID NOs: 53-79, or one of the sequences set forth in SEQ ID NOs: 351-396.

In some examples, the first ABD includes an HCVD comprising the sequence of SEQ ID NO: 342, and an LCVD comprising the sequence of SEQ ID NO: 5, one of the sequences as set forth in SEQ ID NOs: 53-79, or one of the sequences set forth in SEQ ID NOs: 351-396.

In some examples, the first ABD includes an HCVD comprising the sequence of SEQ ID NO: 343, and an LCVD comprising the sequence of SEQ ID NO: 5, one of the sequences as set forth in SEQ ID NOs: 53-79, or one of the sequences set forth in SEQ ID NOs: 351-396.

In some examples, the first ABD includes an HCVD comprising the sequence of SEQ ID NO: 344, and an LCVD comprising the sequence of SEQ ID NO: 5, one of the sequences as set forth in SEQ ID NOs: 53-79, or one of the sequences set forth in SEQ ID NOs: 351-396.

In some examples, the first ABD includes an HCVD comprising the sequence of SEQ ID NO: 345, and an LCVD comprising the sequence of SEQ ID NO: 5, one of the sequences as set forth in SEQ ID NOs: 53-79, or one of the sequences set forth in SEQ ID NOs: 351-396.

In some examples, the first ABD includes an HCVD comprising the sequence of SEQ ID NO: 346, and an LCVD comprising the sequence of SEQ ID NO: 5, one of the sequences as set forth in SEQ ID NOs: 53-79, or one of the sequences set forth in SEQ ID NOs: 351-396.

In some examples, the first ABD includes an HCVD comprising the sequence of SEQ ID NO: 347, and an LCVD comprising the sequence of SEQ ID NO: 5, one of the sequences as set forth in SEQ ID NOs: 53-79, or one of the sequences set forth in SEQ ID NOs: 351-396.

In some examples, the first ABD includes an HCVD comprising the sequence of SEQ ID NO: 348, and an LCVD comprising the sequence of SEQ ID NO: 5, one of the sequences as set forth in SEQ ID NOs: 53-79, or one of the sequences set forth in SEQ ID NOs: 351-396.

In some examples, the first ABD includes an HCVD comprising the sequence of SEQ ID NO: 349, and an LCVD comprising the sequence of SEQ ID NO: 5, one of the sequences as set forth in SEQ ID NOs: 53-79, or one of the sequences set forth in SEQ ID NOs: 351-396.

In some examples, the first ABD includes an HCVD comprising the sequence of SEQ ID NO: 350, and an LCVD comprising the sequence of SEQ ID NO: 5, one of the sequences as set forth in SEQ ID NOs: 53-79, or one of the sequences set forth in SEQ ID NOs: 351-396.

In some examples, the first ABD includes an LCVD comprising the sequence of SEQ ID NO: 5, and an HCVD comprising the sequence of SEQ ID NO: 4, one of the sequences set forth in SEQ ID NOs: 12-52, or one of the sequences set forth in SEQ ID NOs: 313-350.

In some examples, the first ABD includes an LCVD comprising the sequence of SEQ ID NO: 53, and an HCVD comprising the sequence of SEQ ID NO: 4, one of the sequences set forth in SEQ ID NOs: 12-52, or one of the sequences set forth in SEQ ID NOs: 313-350.

In some examples, the first ABD includes an LCVD comprising the sequence of SEQ ID NO: 54, and an HCVD comprising the sequence of SEQ ID NO: 4, one of the sequences set forth in SEQ ID NOs: 12-52, or one of the sequences set forth in SEQ ID NOs: 313-350.

In some examples, the first ABD includes an LCVD comprising the sequence of SEQ ID NO: 55, and an HCVD comprising the sequence of SEQ ID NO: 4, one of the sequences set forth in SEQ ID NOs: 12-52, or one of the sequences set forth in SEQ ID NOs: 313-350.

In some examples, the first ABD includes an LCVD comprising the sequence of SEQ ID NO: 56, and an HCVD comprising the sequence of SEQ ID NO: 4, one of the sequences set forth in SEQ ID NOs: 12-52, or one of the sequences set forth in SEQ ID NOs: 313-350.

In some examples, the first ABD includes an LCVD comprising the sequence of SEQ ID NO: 57, and an HCVD comprising the sequence of SEQ ID NO: 4, one of the sequences set forth in SEQ ID NOs: 12-52, or one of the sequences set forth in SEQ ID NOs: 313-350.

In some examples, the first ABD includes an LCVD comprising the sequence of SEQ ID NO: 58, and an HCVD comprising the sequence of SEQ ID NO: 4, one of the sequences set forth in SEQ ID NOs: 12-52, or one of the sequences set forth in SEQ ID NOs: 313-350.

In some examples, the first ABD includes an LCVD comprising the sequence of SEQ ID NO: 59, and an HCVD comprising the sequence of SEQ ID NO: 4, one of the sequences set forth in SEQ ID NOs: 12-52, or one of the sequences set forth in SEQ ID NOs: 313-350.

In some examples, the first ABD includes an LCVD comprising the sequence of SEQ ID NO: 60, and an HCVD comprising the sequence of SEQ ID NO: 4, one of the sequences set forth in SEQ ID NOs: 12-52, or one of the sequences set forth in SEQ ID NOs: 313-350.

In some examples, the first ABD includes an LCVD comprising the sequence of SEQ ID NO: 61, and an HCVD comprising the sequence of SEQ ID NO: 4, one of the sequences set forth in SEQ ID NOs: 12-52, or one of the sequences set forth in SEQ ID NOs: 313-350.

In some examples, the first ABD includes an LCVD comprising the sequence of SEQ ID NO: 62, and an HCVD comprising the sequence of SEQ ID NO: 4, one of the sequences set forth in SEQ ID NOs: 12-52, or one of the sequences set forth in SEQ ID NOs: 313-350.

In some examples, the first ABD includes an LCVD comprising the sequence of SEQ ID NO: 63, and an HCVD comprising the sequence of SEQ ID NO: 4, one of the sequences set forth in SEQ ID NOs: 12-52, or one of the sequences set forth in SEQ ID NOs: 313-350.

In some examples, the first ABD includes an LCVD comprising the sequence of SEQ ID NO: 64, and an HCVD comprising the sequence of SEQ ID NO: 4, one of the sequences set forth in SEQ ID NOs: 12-52, or one of the sequences set forth in SEQ ID NOs: 313-350.

In some examples, the first ABD includes an LCVD comprising the sequence of SEQ ID NO: 65, and an HCVD comprising the sequence of SEQ ID NO: 4, one of the sequences set forth in SEQ ID NOs: 12-52, or one of the sequences set forth in SEQ ID NOs: 313-350.

In some examples, the first ABD includes an LCVD comprising the sequence of SEQ ID NO: 66, and an HCVD comprising the sequence of SEQ ID NO: 4, one of the sequences set forth in SEQ ID NOs: 12-52, or one of the sequences set forth in SEQ ID NOs: 313-350.

In some examples, the first ABD includes an LCVD comprising the sequence of SEQ ID NO: 67, and an HCVD comprising the sequence of SEQ ID NO: 4, one of the sequences set forth in SEQ ID NOs: 12-52, or one of the sequences set forth in SEQ ID NOs: 313-350.

In some examples, the first ABD includes an LCVD comprising the sequence of SEQ ID NO: 68, and an HCVD comprising the sequence of SEQ ID NO: 4, one of the sequences set forth in SEQ ID NOs: 12-52, or one of the sequences set forth in SEQ ID NOs: 313-350.

In some examples, the first ABD includes an LCVD comprising the sequence of SEQ ID NO: 69, and an HCVD comprising the sequence of SEQ ID NO: 4, one of the sequences set forth in SEQ ID NOs: 12-52, or one of the sequences set forth in SEQ ID NOs: 313-350.

In some examples, the first ABD includes an LCVD comprising the sequence of SEQ ID NO: 70, and an HCVD comprising the sequence of SEQ ID NO: 4, one of the sequences set forth in SEQ ID NOs: 12-52, or one of the sequences set forth in SEQ ID NOs: 313-350.

In some examples, the first ABD includes an LCVD comprising the sequence of SEQ ID NO: 71, and an HCVD comprising the sequence of SEQ ID NO: 4, one of the sequences set forth in SEQ ID NOs: 12-52, or one of the sequences set forth in SEQ ID NOs: 313-350.

In some examples, the first ABD includes an LCVD comprising the sequence of SEQ ID NO: 72, and an HCVD comprising the sequence of SEQ ID NO: 4, one of the sequences set forth in SEQ ID NOs: 12-52, or one of the sequences set forth in SEQ ID NOs: 313-350.

In some examples, the first ABD includes an LCVD comprising the sequence of SEQ ID NO: 73, and an HCVD comprising the sequence of SEQ ID NO: 4, one of the sequences set forth in SEQ ID NOs: 12-52, or one of the sequences set forth in SEQ ID NOs: 313-350.

In some examples, the first ABD includes an LCVD comprising the sequence of SEQ ID NO: 74, and an HCVD comprising the sequence of SEQ ID NO: 4, one of the sequences set forth in SEQ ID NOs: 12-52, or one of the sequences set forth in SEQ ID NOs: 313-350.

In some examples, the first ABD includes an LCVD comprising the sequence of SEQ ID NO: 75, and an HCVD comprising the sequence of SEQ ID NO: 4, one of the sequences set forth in SEQ ID NOs: 12-52, or one of the sequences set forth in SEQ ID NOs: 313-350.

In some examples, the first ABD includes an LCVD comprising the sequence of SEQ ID NO: 76, and an HCVD comprising the sequence of SEQ ID NO: 4, one of the sequences set forth in SEQ ID NOs: 12-52, or one of the sequences set forth in SEQ ID NOs: 313-350.

In some examples, the first ABD includes an LCVD comprising the sequence of SEQ ID NO: 77, and an HCVD comprising the sequence of SEQ ID NO: 4, one of the sequences set forth in SEQ ID NOs: 12-52, or one of the sequences set forth in SEQ ID NOs: 313-350.

In some examples, the first ABD includes an LCVD comprising the sequence of SEQ ID NO: 78, and an HCVD comprising the sequence of SEQ ID NO: 4, one of the sequences set forth in SEQ ID NOs: 12-52, or one of the sequences set forth in SEQ ID NOs: 313-350.

In some examples, the first ABD includes an LCVD comprising the sequence of SEQ ID NO: 79, and an HCVD comprising the sequence of SEQ ID NO: 4, one of the sequences set forth in SEQ ID NOs: 12-52, or one of the sequences set forth in SEQ ID NOs: 313-350.

In some examples, the first ABD includes an LCVD comprising the sequence of SEQ ID NO: 351, and an HCVD comprising the sequence of SEQ ID NO: 4, one of the sequences set forth in SEQ ID NOs: 12-52, or one of the sequences set forth in SEQ ID NOs: 313-350.

In some examples, the first ABD includes an LCVD comprising the sequence of SEQ ID NO: 352, and an HCVD comprising the sequence of SEQ ID NO: 4, one of the sequences set forth in SEQ ID NOs: 12-52, or one of the sequences set forth in SEQ ID NOs: 313-350.

In some examples, the first ABD includes an LCVD comprising the sequence of SEQ ID NO: 353, and an HCVD comprising the sequence of SEQ ID NO: 4, one of the sequences set forth in SEQ ID NOs: 12-52, or one of the sequences set forth in SEQ ID NOs: 313-350.

In some examples, the first ABD includes an LCVD comprising the sequence of SEQ ID NO: 354, and an HCVD comprising the sequence of SEQ ID NO: 4, one of the sequences set forth in SEQ ID NOs: 12-52, or one of the sequences set forth in SEQ ID NOs: 313-350.

In some examples, the first ABD includes an LCVD comprising the sequence of SEQ ID NO: 355, and an HCVD comprising the sequence of SEQ ID NO: 4, one of the sequences set forth in SEQ ID NOs: 12-52, or one of the sequences set forth in SEQ ID NOs: 313-350.

In some examples, the first ABD includes an LCVD comprising the sequence of SEQ ID NO: 356, and an HCVD comprising the sequence of SEQ ID NO: 4, one of the sequences set forth in SEQ ID NOs: 12-52, or one of the sequences set forth in SEQ ID NOs: 313-350.

In some examples, the first ABD includes an LCVD comprising the sequence of SEQ ID NO: 357, and an HCVD comprising the sequence of SEQ ID NO: 4, one of the sequences set forth in SEQ ID NOs: 12-52, or one of the sequences set forth in SEQ ID NOs: 313-350.

In some examples, the first ABD includes an LCVD comprising the sequence of SEQ ID NO: 358, and an HCVD comprising the sequence of SEQ ID NO: 4, one of the sequences set forth in SEQ ID NOs: 12-52, or one of the sequences set forth in SEQ ID NOs: 313-350.

In some examples, the first ABD includes an LCVD comprising the sequence of SEQ ID NO: 359, and an HCVD comprising the sequence of SEQ ID NO: 4, one of the sequences set forth in SEQ ID NOs: 12-52, or one of the sequences set forth in SEQ ID NOs: 313-350.

In some examples, the first ABD includes an LCVD comprising the sequence of SEQ ID NO: 360, and an HCVD comprising the sequence of SEQ ID NO: 4, one of the sequences set forth in SEQ ID NOs: 12-52, or one of the sequences set forth in SEQ ID NOs: 313-350.

In some examples, the first ABD includes an LCVD comprising the sequence of SEQ ID NO: 361, and an HCVD comprising the sequence of SEQ ID NO: 4, one of the sequences set forth in SEQ ID NOs: 12-52, or one of the sequences set forth in SEQ ID NOs: 313-350.

In some examples, the first ABD includes an LCVD comprising the sequence of SEQ ID NO: 362, and an HCVD comprising the sequence of SEQ ID NO: 4, one of the sequences set forth in SEQ ID NOs: 12-52, or one of the sequences set forth in SEQ ID NOs: 313-350.

In some examples, the first ABD includes an LCVD comprising the sequence of SEQ ID NO: 363, and an HCVD comprising the sequence of SEQ ID NO: 4, one of the sequences set forth in SEQ ID NOs: 12-52, or one of the sequences set forth in SEQ ID NOs: 313-350.

In some examples, the first ABD includes an LCVD comprising the sequence of SEQ ID NO: 364, and an HCVD comprising the sequence of SEQ ID NO: 4, one of the sequences set forth in SEQ ID NOs: 12-52, or one of the sequences set forth in SEQ ID NOs: 313-350.

In some examples, the first ABD includes an LCVD comprising the sequence of SEQ ID NO: 365, and an HCVD comprising the sequence of SEQ ID NO: 4, one of the sequences set forth in SEQ ID NOs: 12-52, or one of the sequences set forth in SEQ ID NOs: 313-350.

In some examples, the first ABD includes an LCVD comprising the sequence of SEQ ID NO: 366, and an HCVD comprising the sequence of SEQ ID NO: 4, one of the sequences set forth in SEQ ID NOs: 12-52, or one of the sequences set forth in SEQ ID NOs: 313-350.

In some examples, the first ABD includes an LCVD comprising the sequence of SEQ ID NO: 367, and an HCVD comprising the sequence of SEQ ID NO: 4, one of the sequences set forth in SEQ ID NOs: 12-52, or one of the sequences set forth in SEQ ID NOs: 313-350.

In some examples, the first ABD includes an LCVD comprising the sequence of SEQ ID NO: 368, and an HCVD comprising the sequence of SEQ ID NO: 4, one of the sequences set forth in SEQ ID NOs: 12-52, or one of the sequences set forth in SEQ ID NOs: 313-350.

In some examples, the first ABD includes an LCVD comprising the sequence of SEQ ID NO: 369, and an HCVD comprising the sequence of SEQ ID NO: 4, one of the sequences set forth in SEQ ID NOs: 12-52, or one of the sequences set forth in SEQ ID NOs: 313-350.

In some examples, the first ABD includes an LCVD comprising the sequence of SEQ ID NO: 370, and an HCVD comprising the sequence of SEQ ID NO: 4, one of the sequences set forth in SEQ ID NOs: 12-52, or one of the sequences set forth in SEQ ID NOs: 313-350.

In some examples, the first ABD includes an LCVD comprising the sequence of SEQ ID NO: 371, and an HCVD comprising the sequence of SEQ ID NO: 4, one of the sequences set forth in SEQ ID NOs: 12-52, or one of the sequences set forth in SEQ ID NOs: 313-350.

In some examples, the first ABD includes an LCVD comprising the sequence of SEQ ID NO: 372, and an HCVD comprising the sequence of SEQ ID NO: 4, one of the sequences set forth in SEQ ID NOs: 12-52, or one of the sequences set forth in SEQ ID NOs: 313-350.

In some examples, the first ABD includes an LCVD comprising the sequence of SEQ ID NO: 373, and an HCVD comprising the sequence of SEQ ID NO: 4, one of the sequences set forth in SEQ ID NOs: 12-52, or one of the sequences set forth in SEQ ID NOs: 313-350.

In some examples, the first ABD includes an LCVD comprising the sequence of SEQ ID NO: 374, and an HCVD comprising the sequence of SEQ ID NO: 4, one of the sequences set forth in SEQ ID NOs: 12-52, or one of the sequences set forth in SEQ ID NOs: 313-350.

In some examples, the first ABD includes an LCVD comprising the sequence of SEQ ID NO: 375, and an HCVD comprising the sequence of SEQ ID NO: 4, one of the sequences set forth in SEQ ID NOs: 12-52, or one of the sequences set forth in SEQ ID NOs: 313-350.

In some examples, the first ABD includes an LCVD comprising the sequence of SEQ ID NO: 376, and an HCVD comprising the sequence of SEQ ID NO: 4, one of the sequences set forth in SEQ ID NOs: 12-52, or one of the sequences set forth in SEQ ID NOs: 313-350.

In some examples, the first ABD includes an LCVD comprising the sequence of SEQ ID NO: 377, and an HCVD comprising the sequence of SEQ ID NO: 4, one of the sequences set forth in SEQ ID NOs: 12-52, or one of the sequences set forth in SEQ ID NOs: 313-350.

In some examples, the first ABD includes an LCVD comprising the sequence of SEQ ID NO: 378, and an HCVD comprising the sequence of SEQ ID NO: 4, one of the sequences set forth in SEQ ID NOs: 12-52, or one of the sequences set forth in SEQ ID NOs: 313-350.

In some examples, the first ABD includes an LCVD comprising the sequence of SEQ ID NO: 379, and an HCVD comprising the sequence of SEQ ID NO: 4, one of the sequences set forth in SEQ ID NOs: 12-52, or one of the sequences set forth in SEQ ID NOs: 313-350.

In some examples, the first ABD includes an LCVD comprising the sequence of SEQ ID NO: 380, and an HCVD comprising the sequence of SEQ ID NO: 4, one of the sequences set forth in SEQ ID NOs: 12-52, or one of the sequences set forth in SEQ ID NOs: 313-350.

In some examples, the first ABD includes an LCVD comprising the sequence of SEQ ID NO: 381, and an HCVD comprising the sequence of SEQ ID NO: 4, one of the sequences set forth in SEQ ID NOs: 12-52, or one of the sequences set forth in SEQ ID NOs: 313-350.

In some examples, the first ABD includes an LCVD comprising the sequence of SEQ ID NO: 382, and an HCVD comprising the sequence of SEQ ID NO: 4, one of the sequences set forth in SEQ ID NOs: 12-52, or one of the sequences set forth in SEQ ID NOs: 313-350.

In some examples, the first ABD includes an LCVD comprising the sequence of SEQ ID NO: 383, and an HCVD comprising the sequence of SEQ ID NO: 4, one of the sequences set forth in SEQ ID NOs: 12-52, or one of the sequences set forth in SEQ ID NOs: 313-350.

In some examples, the first ABD includes an LCVD comprising the sequence of SEQ ID NO: 384, and an HCVD comprising the sequence of SEQ ID NO: 4, one of the sequences set forth in SEQ ID NOs: 12-52, or one of the sequences set forth in SEQ ID NOs: 313-350.

In some examples, the first ABD includes an LCVD comprising the sequence of SEQ ID NO: 385, and an HCVD comprising the sequence of SEQ ID NO: 4, one of the sequences set forth in SEQ ID NOs: 12-52, or one of the sequences set forth in SEQ ID NOs: 313-350.

In some examples, the first ABD includes an LCVD comprising the sequence of SEQ ID NO: 386, and an HCVD comprising the sequence of SEQ ID NO: 4, one of the sequences set forth in SEQ ID NOs: 12-52, or one of the sequences set forth in SEQ ID NOs: 313-350.

In some examples, the first ABD includes an LCVD comprising the sequence of SEQ ID NO: 387, and an HCVD comprising the sequence of SEQ ID NO: 4, one of the sequences set forth in SEQ ID NOs: 12-52, or one of the sequences set forth in SEQ ID NOs: 313-350.

In some examples, the first ABD includes an LCVD comprising the sequence of SEQ ID NO: 388, and an HCVD comprising the sequence of SEQ ID NO: 4, one of the sequences set forth in SEQ ID NOs: 12-52, or one of the sequences set forth in SEQ ID NOs: 313-350.

In some examples, the first ABD includes an LCVD comprising the sequence of SEQ ID NO: 389, and an HCVD comprising the sequence of SEQ ID NO: 4, one of the sequences set forth in SEQ ID NOs: 12-52, or one of the sequences set forth in SEQ ID NOs: 313-350.

In some examples, the first ABD includes an LCVD comprising the sequence of SEQ ID NO: 390, and an HCVD comprising the sequence of SEQ ID NO: 4, one of the sequences set forth in SEQ ID NOs: 12-52, or one of the sequences set forth in SEQ ID NOs: 313-350.

In some examples, the first ABD includes an LCVD comprising the sequence of SEQ ID NO: 391, and an HCVD comprising the sequence of SEQ ID NO: 4, one of the sequences set forth in SEQ ID NOs: 12-52, or one of the sequences set forth in SEQ ID NOs: 313-350.

In some examples, the first ABD includes an LCVD comprising the sequence of SEQ ID NO: 392, and an HCVD comprising the sequence of SEQ ID NO: 4, one of the sequences set forth in SEQ ID NOs: 12-52, or one of the sequences set forth in SEQ ID NOs: 313-350.

In some examples, the first ABD includes an LCVD comprising the sequence of SEQ ID NO: 393, and an HCVD comprising the sequence of SEQ ID NO: 4, one of the sequences set forth in SEQ ID NOs: 12-52, or one of the sequences set forth in SEQ ID NOs: 313-350.

In some examples, the first ABD includes an LCVD comprising the sequence of SEQ ID NO: 394, and an HCVD comprising the sequence of SEQ ID NO: 4, one of the sequences set forth in SEQ ID NOs: 12-52, or one of the sequences set forth in SEQ ID NOs: 313-350.

In some examples, the first ABD includes an LCVD comprising the sequence of SEQ ID NO: 395, and an HCVD comprising the sequence of SEQ ID NO: 4, one of the sequences set forth in SEQ ID NOs: 12-52, or one of the sequences set forth in SEQ ID NOs: 313-350.

In some examples, the first ABD includes an LCVD comprising the sequence of SEQ ID NO: 396, and an HCVD comprising the sequence of SEQ ID NO: 4, one of the sequences set forth in SEQ ID NOs: 12-52, or one of the sequences set forth in SEQ ID NOs: 313-350.

In some examples, the first ABD includes an HCVD of datopotamab with one or more amino acids substituted with a histidine. In some examples, the first ABD includes an LCVD of datopotamab with one or more amino acids substituted with a histidine. In some examples, the first ABD includes an HCVD of datopotamab with one or more amino acids substituted with a histidine; and an LCVD of datopotamab with one or more amino acids substituted with a histidine. In some examples, the HCVD of datopotamab comprises SEQ ID NO: 80. In some examples, the LCVD of datopotamab comprises SEQ ID NO: 81. In each of the foregoing examples, an alanine may be substituted where a histidine was already present.

In some examples, the first ABD comprises an HCVD comprising a CDR1, a CDR2, and a CDR3 of SEQ ID NOs: 82, 83, and 84, respectively, with collectively a total of one or more amino acid positions in SEQ ID NOs: 82, 83, and 84 substituted with a histidine. In some examples, the first ABD comprises an LCVD comprising a CDR1, a CDR2, and a CDR3 of SEQ ID NOs: 85, 86, and 87, respectively, with collectively a total of one or more amino acid positions in SEQ ID NOs: 85, 86, and 87 substituted with a histidine. In some examples, the first ABD includes: an HCVD comprising a CDR1, a CDR2, and a CDR3 of SEQ ID NOs: 82, 83, and 84, respectively, with collectively a total of one or more amino acid positions in NOs: 82, 83, and 84 substituted with a histidine; and an LCVD comprising a CDR1, a CDR2, and a CDR3 of SEQ ID NOs: 85, 86, and 87, respectively, with collectively a total of one or more amino acid positions in SEQ ID NOs: 85, 86, and 87 substituted with a histidine. In each of the foregoing examples, an alanine may be substituted where a histidine was already present.

In some examples, the first ABD includes an HCVD that is at least 90%, 95%, or 98% identical to SEQ ID NO: 80, where the HCVD includes a histidine at one or more amino acid positions in SEQ ID NO: 80 selected from: 29, 53, 55, 64, 65, 98, 101, 104, 105, 107, and 108. In some examples, the first ABD includes an LCVD that is at least 90%, 95%, or 98% identical to SEQ ID NO: 81, where the LCVD includes a histidine at one or more amino acid positions in SEQ ID NO: 81 selected from: 26, 28, 29, 30, 31, 33, 34, 50, 51, 52, 53, 54, 89, 93, 94, and 95. In some examples, the first ABD includes an HCVD that is at least 90%, 95%, or 98% identical to SEQ ID NO: 80, where the HCVD includes a histidine at one or more amino acid positions in SEQ ID NO: 80 selected from: 29, 53, 55, 64, 65, 98, 101, 104, 105, 107, and 108, and an LCVD that is at least 90%, 95%, or 98% identical to SEQ ID NO. 81, where the LCVD includes a histidine at one or more amino acid positions in SEQ ID NO: 81 selected from: 26, 28, 29, 30, 31, 33, 34, 50, 51, 52, 53, 54, 89, 93, 94, and 95.

In some examples, an HCVD includes an HCVD that is at least 90%, 95%, or 98% identical to SEQ ID NO: 80, where the HCVD includes a histidine at any of the specific combinations of two or more amino acid positions in SEQ ID NO: 80 listed in Table 3.

In some examples, the first ABD includes an HCVD that is at least 90% (e.g., at least 92%, 94%, 96%, 98%, at least 99%, or 100%) identical to SEQ ID NO. 80, where the HCVD includes an alanine at position 54 in SEQ ID NO: 80.

In some examples the first ABD includes: an HCVD that is at least 90% (e.g., at least 92%, 94%, 96%, 98%, at least 99%, or 100%) identical to SEQ ID NO: 80, where the HCVD includes an alanine at position 54 in SEQ ID NO: 80, and an LCVD that is at least 90% (e.g., at least 92%, 94%, 96%, 98%, at least 99%/o, or 100%) identical to SEQ ID NO: 81, where the LCVD includes a histidine at one or more amino acid positions in SEQ ID NO: 81 selected from: 26, 28, 29, 30, 31, 33, 34, 50, 51, 52, 53, 54, 89, 93, 94, and 95.

TABLE 3
Exemplary Combinations of Amino Acid Positions in SEQ ID NO: 80 that can be
Substituted with Histidine (or an Alanine (A) in the case of position 54)

| | Column | | | | | | | | | | | |
|---|---|---|---|---|---|---|---|---|---|---|---|---|
| | 1 | 2 | 3 | 4 | 5 | 6 | 7 | 8 | 9 | 10 | 11 | 12 |
| SEQ 80 AA Position | 29 | 53 | 55 | 64 | 65 | 98 | 101 | 104 | 105 | 107 | 108 | 54 (A) |

Exemplary 2, 3, and 4 AA position combinations that can be substituted with histidine (each
combination optionally including an alanine at position 54 of SEQ ID NO: 80)

| | |
|---|---|
| A | Each and every unique 2-position combination of X & Y, wherein X and Y are each |
| | selected (without replacement) from a SEQ ID NO: 80 AA position indicated in columns |
| | 1-12. Expressed another way, the combinations of positions recited in the following |
| | combinations of columns: 1 + 2, 1 + 3, 1 + 4, 1 + 5, 1 + 6, 1 + 7, 1 + 8, 1 + 9, 1 + 10, 1 + 11, 1 + 12, |
| | 2 + 3, 2 + 4, 2 + 5, 2 + 6, 2 + 7, 2 + 8, 2 + 9, 2 + 10, 2 + 11, 2 + 12, 3 + 4, 3 + 5, 3 + 6, 3 + 7, 3 + 8, 3 + 9, |
| | 3 + 10, 3 + 11, 3 + 12, 4 + 5, 4 + 6, 4 + 7, 4 + 8, 4 + 9, 4 + 10, 4 + 11, 4 + 12, 5 + 6, 5 + 7, 5 + 8, 5 + 9, |
| | 5 + 10, 5 + 11, 5 + 12, 6 + 7, 6 + 8, 6 + 9, 6 + 10, 6 + 11, 6 + 12, 7 + 8, 7 + 9, 7 + 10, 7 + 11, 7 + 12, 8 + 9, |
| | 8 + 10, 8 + 11, 8 + 12, 9 + 10, 9 + 11, 9 + 12, 10 + 11, 10 + 12, and 11 + 12. |
| B | Each and every unique 3-position combination of X & Y & Z, wherein X, Y, and Z are |
| | each selected (without replacement) from a SEQ ID NO: 80 AA position indicated in |
| | columns 1-12. Expressed another way, the combinations of positions recited in the |
| | following combinations of columns: 1 + 2 + 3, 1 + 2 + 4, 1 + 2 + 5, 1 + 2 + 6, 1 + 2 + 7, 1 + 2 + 8, |
| | 1 + 2 + 9, 1 + 2 + 10, 1 + 2 + 11, 1 + 2 + 12, 1 + 3 + 4, 1 + 3 + 5, 1 + 3 + 6, 1 + 3 + 7, 1 + 3 + 8, 1 + 3 + 9, |
| | 1 + 3 + 10, 1 + 3 + 11, 1 + 3 + 12, 1 + 4 + 5, 1 + 4 + 6, 1 + 4 + 7, 1 + 4 + 8, 1 + 4 + 9, 1 + 4 + 10, 1 + 4 + 11, |
| | 1 + 4 + 12, 1 + 5 + 6, 1 + 5 + 7, 1 + 5 + 8, 1 + 5 + 9, 1 + 5 + 10, 1 + 5 + 11, 1 + 5 + 12, 1 + 6 + 7, 1 + 6 + 8, |
| | 1 + 6 + 9, 1 + 6 + 10, 1 + 6 + 11, 1 + 6 + 12, 1 + 7 + 8, 1 + 7 + 9, 1 + 7 + 10, 1 + 7 + 11, 1 + 7 + 12, 1 + 8 + 9, |
| | 1 + 8 + 10, 1 + 8 + 11, 1 + 8 + 12, 1 + 9 + 10, 1 + 9 + 11, 1 + 9 + 12, 1 + 10 + 11, 1 + 10 + 12, 1 + 11 + 12, |
| | 2 + 3 + 4, 2 + 3 + 5, 2 + 3 + 6, 2 + 3 + 7, 2 + 3 + 8, 2 + 3 + 9, 2 + 3 + 10, 2 + 3 + 11, 2 + 3 + 12, 2 + 4 + 5, 2 + 4 + 6, |
| | 2 + 4 + 7, 2 + 4 + 8, 2 + 4 + 9, 2 + 4 + 10, 2 + 4 + 11, 2 + 4 + 12, 2 + 5 + 6, 2 + 5 + 7, 2 + 5 + 8, 2 + 5 + 9, |
| | 2 + 5 + 10, 2 + 5 + 11, 2 + 5 + 12, 2 + 6 + 7, 2 + 6 + 8, 2 + 6 + 9, 2 + 6 + 10, 2 + 6 + 11, 2 + 6 + 12, 2 + 7 + 8, |
| | 2 + 7 + 9, 2 + 7 + 10, 2 + 7 + 11, 2 + 7 + 12, 2 + 8 + 9, 2 + 8 + 10, 2 + 8 + 11, 2 + 8 + 12, 2 + 9 + 10, 2 + 9 + 11, |
| | 2 + 9 + 12, 2 + 10 + 11, 2 + 10 + 12, 2 + 11 + 12, 3 + 4 + 5, 3 + 4 + 6, 3 + 4 + 7, 3 + 4 + 8, 3 + 4 + 9, 3 + 4 + 10, |
| | 3 + 4 + 11, 3 + 4 + 12, 3 + 5 + 6, 3 + 5 + 7, 3 + 5 + 8, 3 + 5 + 9, 3 + 5 + 10, 3 + 5 + 11, 3 + 5 + 12, 3 + 6 + 7, |
| | 3 + 6 + 8, 3 + 6 + 9, 3 + 6 + 10, 3 + 6 + 11, 3 + 6 + 12, 3 + 7 + 8, 3 + 7 + 9, 3 + 7 + 10, 3 + 7 + 11, 3 + 7 + 12, |
| | 3 + 8 + 9, 3 + 8 + 10, 3 + 8 + 11, 3 + 8 + 12, 3 + 9 + 10, 3 + 9 + 11, 3 + 9 + 12, 3 + 10 + 11, 3 + 10 + 12, |
| | 3 + 11 + 12, 4 + 5 + 6, 4 + 5 + 7, 4 + 5 + 8, 4 + 5 + 9, 4 + 5 + 10, 4 + 5 + 11, 4 + 5 + 12, 4 + 6 + 7, 4 + 6 + 8, |
| | 4 + 6 + 9, 4 + 6 + 10, 4 + 6 + 11, 4 + 6 + 12, 4 + 7 + 8, 4 + 7 + 9, 4 + 7 + 10, 4 + 7 + 11, 4 + 7 + 12, 4 + 8 + 9, |
| | 4 + 8 + 10, 4 + 8 + 11, 4 + 8 + 12, 4 + 9 + 10, 4 + 9 + 11, 4 + 9 + 12, 4 + 10 + 11, 4 + 10 + 12, 4 + 11 + 12, |
| | 5 + 6 + 7, 5 + 6 + 8, 5 + 6 + 9, 5 + 6 + 10, 5 + 6 + 11, 5 + 6 + 12, 5 + 7 + 8, 5 + 7 + 9, 5 + 7 + 10, 5 + 7 + 11, |
| | 5 + 7 + 12, 5 + 8 + 9, 5 + 8 + 10, 5 + 8 + 11, 5 + 8 + 12, 5 + 9 + 10, 5 + 9 + 11, 5 + 9 + 12, 5 + 10 + 11, |
| | 5 + 10 + 12, 5 + 11 + 12, 6 + 7 + 8, 6 + 7 + 9, 6 + 7 + 10, 6 + 7 + 11, 6 + 7 + 12, 6 + 8 + 9, 6 + 8 + 10, 6 + 8 + 11, |
| | 6 + 8 + 12, 6 + 9 + 10, 6 + 9 + 11, 6 + 9 + 12, 6 + 10 + 11, 6 + 10 + 12, 6 + 11 + 12, 7 + 8 + 9, 7 + 8 + 10, |
| | 7 + 8 + 11, 7 + 8 + 12, 7 + 9 + 10, 7 + 9 + 11, 7 + 9 + 12, 7 + 10 + 11, 7 + 10 + 12, 7 + 11 + 12, 8 + 9 + 10, |
| | 8 + 9 + 11, 8 + 9 + 12, 8 + 10 + 11, 8 + 10 + 12, 8 + 11 + 12, 9 + 10 + 11, 9 + 10 + 12, 9 + 11 + 12, and |
| | 10 + 11 + 12. |
| C | Each and every unique 4-position combination of X & Y & Z & α, wherein X, Y, and Z |
| | are together one of the 3-position combinations recited in “B” (above), and α is selected |
| | from a SEQ ID NO: 80 amino acid position recited in one of columns 1 to 12, without |
| | replacement (i.e. X ≠ Y ≠ Z ≠ α). |

In some examples, the first ABD includes an LCVD that is at least 90%, 95%, or 98% identical to SEQ ID NO: 81, where the LCVD includes a histidine at any of the specific combinations of two or more amino acid positions in SEQ ID NO: 81 listed in Table 4 and/or where the LCVD includes an alanine at position 91 of SEQ ID NO: 81.

In some examples, the first ABD includes an LCVD that is at least 90%, 95%, or 98% identical to SEQ ID NO: 81, where the LCVD includes an alanine at position 91 in SEQ ID NO: 81.

In some examples the first ABD includes: an LCVD that is at least 90%, 95%, or 98% identical to SEQ ID NO: 81, where the LCVD includes an alanine at position 91 in SEQ ID NO: 81, and an HCVD that is at least 90%, 95%, or 98% identical to SEQ ID NO: 80, where the HCVD includes a histidine at one or more amino acid positions in SEQ ID NO: 80 selected from: 29, 53, 55, 64, 65, 98, 101, 104, 105, 107, and 108.

In some examples, the first ABD includes an LCVD that is at least 90%, 95%, or 98% identical to SEQ ID NO: 81, where the LCVD includes a histidine at one or more amino acid positions in SEQ ID NO: 81 selected from: 26, 28, 29, 30, 31, 33, 34, 50, 51, 52, 53, 54, 89, 93, 94, and 95 and/or where the LCVD includes an alanine at position 91 in SEQ ID NO: 81. In some examples the first ABD includes: an LCVD that is at least 90%, 95%, or 98% identical to SEQ ID NO: 81, where the LCVD includes a histidine at one or more amino acid positions in SEQ ID NO: 81 selected from: 26, 28, 29, 30, 31, 33, 34, 50, 51, 52, 53, 54, 89, 93, 94, and 95 and/or where the LCVD includes an alanine at position 91 in SEQ ID NO: 81, and an HCVD that is at least 90%, 95%, or 98% identical to SEQ ID NO: 80, where the HCVD includes a histidine at one or more amino acid positions in SEQ ID NO: 80 selected from: 29, 53, 55, 64, 65, 98, 101, 104, 105, 107, and 108.

In some examples the first ABD includes an LCVD that is at least 90%, 95%, or 98% identical to SEQ ID NO: 81, where the LCVD includes a histidine at one or more amino acid positions in SEQ ID NO: 81 selected from: 26, 28, 29, 30, 31, 33, 34, 50, 51, 52, 53, 54, 89, 93, 94, and 95 and/or where the LCVD includes an alanine at position 91 in SEQ ID NO: 81, and an HCVD that is at least 90/c, 95%, or 98% identical to SEQ ID NO: 80, where the HCVD includes a histidine at one or more amino acid positions in SEQ ID NO: 80 selected from: 29, 53, 55, 64, 65, 98, 101, 104, 105, 107, and 108 and/or where the HCVD includes an alanine in position 54 of SEQ ID NO: 80.

TABLE 4
Exemplary Combinations of Amino Acid Positions in SEQ ID NO: 81 that can be Substituted 7

| | Column | | | | | | | | | | | | | | | | |
|---|---|---|---|---|---|---|---|---|---|---|---|---|---|---|---|---|---|
| | 1 | 2 | 3 | 4 | 5 | 6 | 7 | 8 | 9 | 10 | 11 | 12 | 13 | 14 | 15 | 16 | 17 |
| SEQ 81 AA Position | 26 | 28 | 29 | 30 | 31 | 33 | 34 | 50 | 51 | 52 | 53 | 54 | 89 | 93 | 94 | 95 | 91 (A) |

Exemplary 2, 3, and 4 AA position combinations that can be substituted with histidine (or an alanine at position 91 of SEQ ID NO: 81)

| | |
|---|---|
| A | Each and every unique 2-position combination of X & Y, wherein X and Y are each |
| | selected (without replacement) from a SEQ ID NO: 81 AA position indicated in columns |
| | 1-17. Expressed another way, the combinations of positions recited in the following |
| | combinations of columns: 1 + 2, 1 + 3, 1 + 4, 1 + 5, 1 + 6, 1 + 7, 1 + 8, 1 + 9, 1 + 10, 1 + 11, 1 + 12, |
| | 1 + 13, 1 + 14, 1 + 15, 1 + 16, 1 + 17, 2 + 3, 2 + 4, 2 + 5, 2 + 6, 2 + 7, 2 + 8, 2 + 9, 2 + 10, 2 + 11, 2 + 12, |
| | 2 + 13, 2 + 14, 2 + 15, 2 + 16, 2 + 17, 3 + 4, 3 + 5, 3 + 6, 3 + 7, 3 + 8, 3 + 9, 3 + 10, 3 + 11, 3 + 12, 3 + 13, |
| | 3 + 14, 3 + 15, 3 + 16, 3 + 17, 4 + 5, 4 + 6, 4 + 7, 4 + 8, 4 + 9, 4 + 10, 4 + 11, 4 + 12, 4 + 13, 4 + 14, 4 + 15, |
| | 4 + 16, 4 + 17, 5 + 6, 5 + 7, 5 + 8, 5 + 9, 5 + 10, 5 + 11, 5 + 12, 5 + 13, 5 + 14, 5 + 15, 5 + 16, 5 + 17, 6 + 7, |
| | 6 + 8, 6 + 9, 6 + 10, 6 + 11, 6 + 12, 6 + 13, 6 + 14, 6 + 15, 6 + 16, 6 + 17, 7 + 8, 7 + 9, 7 + 10, 7 + 11, 7 + 12, |
| | 7 + 13, 7 + 14, 7 + 15, 7 + 16, 7 + 17, 8 + 9, 8 + 10, 8 + 11, 8 + 12, 8 + 13, 8 + 14, 8 + 15, 8 + 16, 8 + 17, |
| | 9 + 10, 9 + 11, 9 + 12, 9 + 13, 9 + 14, 9 + 15, 9 + 16, 9 + 17, 10 + 11, 10 + 12, 10 + 13, 10 + 14, 10 + 15, |
| | 10 + 16, 10 + 17, 11 + 12, 11 + 13, 11 + 14, 11 + 15, 11 + 16, 11 + 17, 12 + 13, 12 + 14, 12 + 15, 12 + 16, |
| | 12 + 17, 13 + 14, 13 + 15, 13 + 16, 13 + 17, 14 + 15, 14 + 16, 14 + 17, 15 + 16, 15 + 17, and 16 + 17. |
| B | Each and every unique 3-position combination of X & Y & Z, wherein X, Y, and Z are |
| | each selected (without replacement) from a SEQ ID NO: 81 AA position indicated in |
| | columns 1-17. Expressed another way, the combinations of positions recited in the |
| | following combinations of columns: 1 + 2 + 3, 1 + 2 + 4, 1 + 2 + 5, 1 + 2 + 6, 1 + 2 + 7, 1 + 2 + 8, 1 + 2 + 9, |
| | 1 + 2 + 10, 1 + 2 + 11, 1 + 2 + 12, 1 + 2 + 13, 1 + 2 + 14, 1 + 2 + 15, 1 + 2 + 16, 1 + 2 + 17, 1 + 3 + 4, 1 + 3 + 5, |
| | 1 + 3 + 6, 1 + 3 + 7, 1 + 3 + 8, 1 + 3 + 9, 1 + 3 + 10, 1 + 3 + 11, 1 + 3 + 12, 1 + 3 + 13, 1 + 3 + 14, 1 + 3 + 15, |
| | 1 + 3 + 16, 1 + 3 + 17, 1 + 4 + 5, 1 + 4 + 6, 1 + 4 + 7, 1 + 4 + 8, 1 + 4 + 9, 1 + 4 + 10, 1 + 4 + 11, 1 + 4 + 12, |
| | 1 + 4 + 13, 1 + 4 + 14, 1 + 4 + 15, 1 + 4 + 16, 1 + 4 + 17, 1 + 5 + 6, 1 + 5 + 7, 1 + 5 + 8, 1 + 5 + 9, 1 + 5 + 10, |
| | 1 + 5 + 11, 1 + 5 + 12, 1 + 5 + 13, 1 + 5 + 14, 1 + 5 + 15, 1 + 5 + 16, 1 + 5 + 17, 1 + 6 + 7, 1 + 6 + 8, 1 + 6 + 9, |
| | 1 + 6 + 10, 1 + 6 + 11, 1 + 6 + 12, 1 + 6 + 13, 1 + 6 + 14, 1 + 6 + 15, 1 + 6 + 16, 1 + 6 + 17, 1 + 7 + 8, 1 + 7 + 9, |
| | 1 + 7 + 10, 1 + 7 + 11, 1 + 7 + 12, 1 + 7 + 13, 1 + 7 + 14, 1 + 7 + 15, 1 + 7 + 16, 1 + 7 + 17, 1 + 8 + 9, 1 + 8 + 10, |
| | 1 + 8 + 11, 1 + 8 + 12, 1 + 8 + 13, 1 + 8 + 14, 1 + 8 + 15, 1 + 8 + 16, 1 + 8 + 17, 1 + 9 + 10, 1 + 9 + 11, 1 + 9 + 12, |
| | 1 + 9 + 13, 1 + 9 + 14, 1 + 9 + 15, 1 + 9 + 16, 1 + 9 + 17, 1 + 10 + 11, 1 + 10 + 12, 1 + 10 + 13, 1 + 10 + 14, |
| | 1 + 10 + 15, 1 + 10 + 16, 1 + 10 + 17, 1 + 11 + 12, 1 + 11 + 13, 1 + 11 + 14, 1 + 11 + 15, 1 + 11 + 16, |
| | 1 + 11 + 17, 1 + 12 + 13, 1 + 12 + 14, 1 + 12 + 15, 1 + 12 + 16, 1 + 12 + 17, 1 + 13 + 14, 1 + 13 + 15, |
| | 1 + 13 + 16, 1 + 13 + 17, 1 + 14 + 15, 1 + 14 + 16, 1 + 14 + 17, 1 + 15 + 16, 1 + 15 + 17, 1 + 16 + 17, 2 + 3 + 4, |
| | 2 + 3 + 5, 2 + 3 + 6, 2 + 3 + 7, 2 + 3 + 8, 2 + 3 + 9, 2 + 3 + 10, 2 + 3 + 11, 2 + 3 + 12, 2 + 3 + 13, 2 + 3 + 14, |
| | 2 + 3 + 15, 2 + 3 + 16, 2 + 3 + 17, 2 + 4 + 5, 2 + 4 + 6, 2 + 4 + 7, 2 + 4 + 8, 2 + 4 + 9, 2 + 4 + 10, 2 + 4 + 11, |
| | 2 + 4 + 12, 2 + 4 + 13, 2 + 4 + 14, 2 + 4 + 15, 2 + 4 + 16, 2 + 4 + 17, 2 + 5 + 6, 2 + 5 + 7, 2 + 5 + 8, 2 + 5 + 9, |
| | 2 + 5 + 10, 2 + 5 + 11, 2 + 5 + 12, 2 + 5 + 13, 2 + 5 + 14, 2 + 5 + 15, 2 + 5 + 16, 2 + 5 + 17, 2 + 6 + 7, 2 + 6 + 8, |
| | 2 + 6 + 9, 2 + 6 + 10, 2 + 6 + 11, 2 + 6 + 12, 2 + 6 + 13, 2 + 6 + 14, 2 + 6 + 15, 2 + 6 + 16, 2 + 6 + 17, 2 + 7 + 8, |
| | 2 + 7 + 9, 2 + 7 + 10, 2 + 7 + 11, 2 + 7 + 12, 2 + 7 + 13, 2 + 7 + 14, 2 + 7 + 15, 2 + 7 + 16, 2 + 7 + 17, 2 + 8 + 9, |
| | 2 + 8 + 10, 2 + 8 + 11, 2 + 8 + 12, 2 + 8 + 13, 2 + 8 + 14, 2 + 8 + 15, 2 + 8 + 16, 2 + 8 + 17, 2 + 9 + 10, 2 + 9 + 11, |
| | 2 + 9 + 12, 2 + 9 + 13, 2 + 9 + 14, 2 + 9 + 15, 2 + 9 + 16, 2 + 9 + 17, 2 + 10 + 11, 2 + 10 + 12, 2 + 10 + 13, |
| | 2 + 10 + 14, 2 + 10 + 15, 2 + 10 + 16, 2 + 10 + 17, 2 + 11 + 12, 2 + 11 + 13, 2 + 11 + 14, 2 + 11 + 15, |
| | 2 + 11 + 16, 2 + 11 + 17, 2 + 12 + 13, 2 + 12 + 14, 2 + 12 + 15, 2 + 12 + 16, 2 + 12 + 17, 2 + 13 + 14, |
| | 2 + 13 + 15, 2 + 13 + 16, 2 + 13 + 17, 2 + 14 + 15, 2 + 14 + 16, 2 + 14 + 17, 2 + 15 + 16, 2 + 15 + 17, |
| | 2 + 16 + 17, 3 + 4 + 5, 3 + 4 + 6, 3 + 4 + 7, 3 + 4 + 8, 3 + 4 + 9, 3 + 4 + 10, 3 + 4 + 11, 3 + 4 + 12, 3 + 4 + 13, |
| | 3 + 4 + 14, 3 + 4 + 15, 3 + 4 + 16, 3 + 4 + 17, 3 + 5 + 6, 3 + 5 + 7, 3 + 5 + 8, 3 + 5 + 9, 3 + 5 + 10, 3 + 5 + 11, |
| | 3 + 5 + 12, 3 + 5 + 13, 3 + 5 + 14, 3 + 5 + 15, 3 + 5 + 16, 3 + 5 + 17, 3 + 6 + 7, 3 + 6 + 8, 3 + 6 + 9, 3 + 6 + 10, |
| | 3 + 6 + 11, 3 + 6 + 12, 3 + 6 + 13, 3 + 6 + 14, 3 + 6 + 15, 3 + 6 + 16, 3 + 6 + 17, 3 + 7 + 8, 3 + 7 + 9, 3 + 7 + 10, |
| | 3 + 7 + 11, 3 + 7 + 12. 3 + 7 + 13, 3 + 7 + 14, 3 + 7 + 15, 3 + 7 + 16, 3 + 7 + 17, 3 + 8 + 9, 3 + 8 + 10, 3 + 8 + 11, |
| | 3 + 8 + 12, 3 + 8 + 13, 3 + 8 + 14, 3 + 8 + 15, 3 + 8 + 16, 3 + 8 + 17, 3 + 9 + 10, 3 + 9 + 11, 3 + 9 + 12, 3 + 9 + 13, |
| | 3 + 9 + 14, 3 + 9 + 15, 3 + 9 + 16, 3 + 9 + 17, 3 + 10 + 11, 3 + 10 + 12, 3 + 10 + 13, 3 + 10 + 14, 3 + 10 + 15, |
| | 3 + 10 + 16, 3 + 10 + 17, 3 + 11 + 12, 3 + 11 + 13, 3 + 11 + 14, 3 + 11 + 15, 3 + 11 + 16, 3 + 11 + 17, |
| | 3 + 12 + 13, 3 + 12 + 14, 3 + 12 + 15, 3 + 12 + 16, 3 + 12 + 17, 3 + 13 + 14, 3 + 13 + 15, 3 + 13 + 16, |
| | 3 + 13 + 17, 3 + 14 + 15, 3 + 14 + 16, 3 + 14 + 17, 3 + 15 + 16, 3 + 15 + 17, 3 + 16 + 17, 4 + 5 + 6, 4 + 5 + 7, |
| | 4 + 5 + 8, 4 + 5 + 9, 4 + 5 + 10, 4 + 5 + 11, 4 + 5 + 12, 4 + 5 + 13, 4 + 5 + 14, 4 + 5 + 15, 4 + 5 + 16, 4 + 5 + 17, |
| | 4 + 6 + 7, 4 + 6 + 8, 4 + 6 + 9, 4 + 6 + 10, 4 + 6 + 11, 4 + 6 + 12, 4 + 6 + 13, 4 + 6 + 14, 4 + 6 + 15, 4 + 6 + 16, |
| | 4 + 6 + 17, 4 + 7 + 8, 4 + 7 + 9, 4 + 7 + 10, 4 + 7 + 11, 4 + 7 + 12, 4 + 7 + 13, 4 + 7 + 14, 4 + 7 + 15, 4 + 7 + 16, |
| | 4 + 7 + 17, 4 + 8 + 9, 4 + 8 + 10, 4 + 8 + 11, 4 + 8 + 12, 4 + 8 + 13, 4 + 8 + 14, 4 + 8 + 15, 4 + 8 + 16, 4 + 8 + 17, |
| | 4 + 9 + 10, 4 + 9 + 11, 4 + 9 + 12, 4 + 9 + 13, 4 + 9 + 14, 4 + 9 + 15, 4 + 9 + 16, 4 + 9 + 17, 4 + 10 + 11, |
| | 4 + 10 + 12, 4 + 10 + 13, 4 + 10 + 14, 4 + 10 + 15, 4 + 10 + 16, 4 + 10 + 17, 4 + 11 + 12, 4 + 11 + 13, |
| | 4 + 11 + 14, 4 + 11 + 15, 4 + 11 + 16, 4 + 11 + 17, 4 + 12 + 13, 4 + 12 + 14, 4 + 12 + 15, 4 + 12 + 16, |
| | 4 + 12 + 17, 4 + 13 + 14, 4 + 13 + 15, 4 + 13 + 16, 4 + 13 + 17, 4 + 14 + 15, 4 + 14 + 16, 4 + 14 + 17, |
| | 4 + 15 + 16, 4 + 15 + 17, 4 + 16 + 17, 5 + 6 + 7, 5 + 6 + 8, 5 + 6 + 9, 5 + 6 + 10, 5 + 6 + 11, 5 + 6 + 12, 5 + 6 + 13, |
| | 5 + 6 + 14, 5 + 6 + 15, 5 + 6 + 16, 5 + 6 + 17, 5 + 7 + 8, 5 + 7 + 9, 5 + 7 + 10, 5 + 7 + 11, 5 + 7 + 12, 5 + 7 + 13, |
| | 5 + 7 + 14, 5 + 7 + 15, 5 + 7 + 16, 5 + 7 + 17, 5 + 8 + 9, 5 + 8 + 10, 5 + 8 + 11, 5 + 8 + 12, 5 + 8 + 13, 5 + 8 + 14, |
| | 5 + 8 + 15, 5 + 8 + 16, 5 + 8 + 17, 5 + 9 + 10, 5 + 9 + 11, 5 + 9 + 12, 5 + 9 + 13, 5 + 9 + 14, 5 + 9 + 15, 5 + 9 + 16, |
| | 5 + 9 + 17, 5 + 10 + 11, 5 + 10 + 12, 5 + 10 + 13, 5 + 10 + 14, 5 + 10 + 15, 5 + 10 + 16, 5 + 10 + 17, 5 + 11 + 12, |
| | 5 + 11 + 13, 5 + 11 + 14, 5 + 11 + 15, 5 + 11 + 16, 5 + 11 + 17, 5 + 12 + 13, 5 + 12 + 14, 5 + 12 + 15, |
| | 5 + 12 + 16, 5 + 12 + 17, 5 + 13 + 14, 5 + 13 + 15, 5 + 13 + 16, 5 + 13 + 17, 5 + 14 + 15, 5 + 14 + 16, |
| | 5 + 14 + 17, 5 + 15 + 16, 5 + 15 + 17, 5 + 16 + 17, 6 + 7 + 8, 6 + 7 + 9, 6 + 7 + 10, 6 + 7 + 11, 6 + 7 + 12, |
| | 6 + 7 + 13, 6 + 7 + 14, 6 + 7 + 15, 6 + 7 + 16, 6 + 7 + 17, 6 + 8 + 9, 6 + 8 + 10, 6 + 8 + 11, 6 + 8 + 12, 6 + 8 + 13, |
| | 6 + 8 + 14, 6 + 8 + 15, 6 + 8 + 16, 6 + 8 + 17, 6 + 9 + 10, 6 + 9 + 11, 6 + 9 + 12, 6 + 9 + 13, 6 + 9 + 14, 6 + 9 + 15, |
| | 6 + 9 + 16, 6 + 9 + 17, 6 + 10 + 11, 6 + 10 + 12, 6 + 10 + 13, 6 + 10 + 14, 6 + 10 + 15, 6 + 10 + 16, 6 + 10 + 17, |
| | 6 + 11 + 12, 6 + 11 + 13, 6 + 11 + 14, 6 + 11 + 15, 6 + 11 + 16, 6 + 11 + 17, 6 + 12 + 13, 6 + 12 + 14, |
| | 6 + 12 + 15, 6 + 12 + 16, 6 + 12 + 17, 6 + 13 + 14, 6 + 13 + 15, 6 + 13 + 16, 6 + 13 + 17, 6 + 14 + 15, |
| | 6 + 14 + 16, 6 + 14 + 17, 6 + 15 + 16, 6 + 15 + 17, 6 + 16 + 17, 7 + 8 + 9, 7 + 8 + 10, 7 + 8 + 11, 7 + 8 + 12, |
| | 7 + 8 + 13, 7 + 8 + 14, 7 + 8 + 15, 7 + 8 + 16, 7 + 8 + 17, 7 + 9 + 10, 7 + 9 + 11, 7 + 9 + 12, 7 + 9 + 13, 7 + 9 + 14, |
| | 7 + 9 + 15, 7 + 9 + 16, 7 + 9 + 17, 7 + 10 + 11, 7 + 10 + 12, 7 + 10 + 13, 7 + 10 + 14, 7 + 10 + 15, 7 + 10 + 16, |
| | 7 + 10 + 17, 7 + 11 + 12, 7 + 11 + 13, 7 + 11 + 14, 7 + 11 + 15, 7 + 11 + 16, 7 + 11 + 17, 7 + 12 + 13, |
| | 7 + 12 + 14, 7 + 12 + 15, 7 + 12 + 16, 7 + 12 + 17, 7 + 13 + 14, 7 + 13 + 15, 7 + 13 + 16, 7 + 13 + 17, |
| | 7 + 14 + 15, 7 + 14 + 16, 7 + 14 + 17, 7 + 15 + 16, 7 + 15 + 17, 7 + 16 + 17, 8 + 9 + 10, 8 + 9 + 11, 8 + 9 + 12, |
| | 8 + 9 + 13, 8 + 9 + 14, 8 + 9 + 15, 8 + 9 + 16, 8 + 9 + 17, 8 + 10 + 11, 8 + 10 + 12, 8 + 10 + 13, 8 + 10 + 14, |
| | 8 + 10 + 15, 8 + 10 + 16, 8 + 10 + 17, 8 + 11 + 12, 8 + 11 + 13, 8 + 11 + 14, 8 + 11 + 15, 8 + 11 + 16, |
| | 8 + 11 + 17, 8 + 12 + 13, 8 + 12 + 14, 8 + 12 + 15, 8 + 12 + 16, 8 + 12 + 17, 8 + 13 + 14, 8 + 13 + 15, |
| | 8 + 13 + 16, 8 + 13 + 17, 8 + 14 + 15, 8 + 14 + 16, 8 + 14 + 17, 8 + 15 + 16, 8 + 15 + 17, 8 + 16 + 17, |
| | 9 + 10 + 11, 9 + 10 + 12, 9 + 10 + 13, 9 + 10 + 14, 9 + 10 + 15, 9 + 10 + 16, 9 + 10 + 17, 9 + 11 + 12, |
| | 9 + 11 + 13, 9 + 11 + 14, 9 + 11 + 15, 9 + 11 + 16, 9 + 11 + 17, 9 + 12 + 13, 9 + 12 + 14, 9 + 12 + 15, |
| | 9 + 12 + 16, 9 + 12 + 17, 9 + 13 + 14, 9 + 13 + 15, 9 + 13 + 16, 9 + 13 + 17, 9 + 14 + 15, 9 + 14 + 16, |
| | 9 + 14 + 17, 9 + 15 + 16, 9 + 15 + 17, 9 + 16 + 17, 10 + 11 + 12, 10 + 11 + 13, 10 + 11 + 14, 10 + 11 + 15, |
| | 10 + 11 + 16, 10 + 11 + 17, 10 + 12 + 13, 10 + 12 + 14, 10 + 12 + 15, 10 + 12 + 16, 10 + 12 + 17, |
| | 10 + 13 + 14, 10 + 13 + 15, 10 + 13 + 16, 10 + 13 + 17, 10 + 14 + 15, 10 + 14 + 16, 10 + 14 + 17, |
| | 10 + 15 + 16, 10 + 15 + 17, 10 + 16 + 17, 11 + 12 + 13, 11 + 12 + 14, 11 + 12 + 15, 11 + 12 + 16, |
| | 11 + 12 + 17, 11 + 13 + 14, 11 + 13 + 15, 11 + 13 + 16, 11 + 13 + 17, 11 + 14 + 15, 11 + 14 + 16, |
| | 11 + 14 + 17, 11 + 15 + 16, 11 + 15 + 17, 11 + 16 + 17, 12 + 13 + 14, 12 + 13 + 15, 12 + 13 + 16, |
| | 12 + 13 + 17, 12 + 14 + 15, 12 + 14 + 16, 12 + 14 + 17, 12 + 15 + 16, 12 + 15 + 17, 12 + 16 + 17, |
| | 13 + 14 + 15, 13 + 14 + 16, 13 + 14 + 17, 13 + 15 + 16, 13 + 15 + 17, 13 + 16 + 17, 14 + 15 + 16, |
| | 14 + 15 + 17, 14 + 16 + 17, and 15 + 16 + 17. |
| C | Each and every unique 4-position combination of X & Y & Z & α, wherein X, Y, and Z are |
| | together one of the 3-position combinations recited in “B” (above), and α is selected from a |
| | SEQ ID NO: 81 amino acid position recited in one of columns 1 to 17, without |
| | replacement (i.e. X ≠ Y ≠ Z ≠ α). |

In some examples, the first ABD includes an HCVD that is at least 90%, 95%, or 98% identical to SEQ ID NO: 80, where the HCVD includes a histidine at any of the specific combinations of two or more amino acid positions in SEQ ID NO: 80 listed in Table 3; and an LCVD that that is at least 90%, 95%, or 98% identical to SEQ ID NO: 81, where the LCVD includes a histidine at any of the specific combinations of two or more amino acid positions in SEQ ID NO. 81 listed in Table 4.

In some examples, the first ABD includes an LCVD that is at least 90%, 95%, or 98% identical to SEQ ID NO: 81, where the LCVD includes a histidine at any of the specific combinations of two or more amino acid positions in SEQ ID NO: 81 listed in Table 4, and/or where the LCVD includes an alanine at position 91 in SEQ ID NO: 81; and an HCVD that that is at least 90%, 95%, or 98% identical to SEQ ID NO: 80, where the HCVD includes a histidine at any of the specific combinations of two or more amino acid positions in SEQ ID NO: 80 listed in Table 3.

In some examples, the first ABD comprises an LCVD comprising SEQ ID NO: 81, and an HCVD that is at least 90%, 95%, or 98% identical to SEQ ID NO: 80, where the HCVD includes a histidine at any of the specific combinations of two or more amino acid positions in SEQ ID NO: 80 listed in Table 3.

In some examples, the first ABD comprises an LCVD that is at least 90%, 95%, or 98% identical to SEQ ID NO: 81, where the LCVD includes a histidine at position 26 in SEQ ID NO: 81; and an HCVD that is at least 90%, 95%, or 98% identical to SEQ ID NO: 80, where the HCVD includes a histidine at any of the specific combinations of two or more amino acid positions in SEQ ID NO: 80 listed in Table 3.

In some examples, the first ABD comprises an LCVD that is at least 90%, 95%, or 98% identical to SEQ ID NO: 81, where the LCVD includes a histidine at position 28 in SEQ ID NO: 81, and an HCVD that is at least 90%, 95%, or 98% identical to SEQ ID NO: 80, where the HCVD includes a histidine at any of the specific combinations of two or more amino acid positions in SEQ ID NO: 80 listed in Table 3.

In some examples, the first ABD comprises an LCVD that is at least 90%, 95%, or 98% identical to SEQ ID NO: 81, where the LCVD includes a histidine at position 29 in SEQ ID NO: 81; and an HCVD that is at least 90%, 95%, or 98% identical to SEQ ID NO: 80, where the HCVD includes a histidine at any of the specific combinations of two or more amino acid positions in SEQ ID NO: 80 listed in Table 3.

In some examples, the first ABD comprises an LCVD that is at least 90%, 95%, or 98% identical to SEQ ID NO: 81, where the LCVD includes an alanine at position 91 in SEQ ID NO: 81; and an HCVD that is at least 90%, 95%, or 98% identical to SEQ ID NO: 80, wherein the HCVD includes a histidine at any of the specific combinations of two or more amino acid positions in SEQ ID NO: 80 listed in Table 3.

In some examples, the first ABD comprises an LCVD that is at least 90%, 95%, or 98% identical to SEQ ID NO: 81, wherein the LCVD includes a histidine at position 30 in SEQ ID NO: 81; and an HCVD that is at least 90%, 95%, or 98% identical to SEQ ID NO: 80, wherein the HCVD includes a histidine at any of the specific combinations of two or more amino acid positions in SEQ ID NO: 80 listed in Table 3.

In some examples, the first ABD comprises an LCVD that is at least 90%, 95%, or 98% identical to SEQ ID NO: 81, wherein the LCVD includes a histidine at position 31 in SEQ ID NO: 81; and an HCVD that is at least 90%, 95%, or 98% identical to SEQ ID NO: 80, where the HCVD includes a histidine at any of the specific combinations of two or more amino acid positions in SEQ ID NO: 80 listed in Table 3.

In some examples, the first ABD comprises an LCVD that is at least 90%, 95%, or 98% identical to SEQ ID NO: 81, wherein the LCVD includes a histidine at position 33 in SEQ ID NO: 81; and an HCVD that is at least 90%, 95%, or 98% identical to SEQ ID NO: 80, where the HCVD includes a histidine at any of the specific combinations of two or more amino acid positions in SEQ ID NO: 80 listed in Table 3.

In some examples, the first ABD comprises an LCVD that is at least 90%, 95%, or 98% identical to SEQ ID NO: 81, wherein the LCVD includes a histidine at position 34 in SEQ ID NO: 81; and an HCVD that is at least 90%, 95%, or 98% identical to SEQ ID NO: 80, where the HCVD includes a histidine at any of the specific combinations of two or more amino acid positions in SEQ ID NO: 80 listed in Table 3.

In some examples, the first ABD comprises an LCVD that is at least 90%, 95%, or 98% identical to SEQ ID NO: 81, wherein the LCVD includes a histidine at position 50 in SEQ ID NO: 81; and an HCVD that is at least 90%, 95%, or 98% identical to SEQ ID NO: 80, where the HCVD includes a histidine at any of the specific combinations of two or more amino acid positions in SEQ ID NO: 80 listed in Table 3.

In some examples, the first ABD comprises an LCVD that is at least 90%, 95%, or 98% identical to SEQ ID NO: 81, wherein the LCVD includes a histidine at position 51 in SEQ ID NO: 81; and an HCVD that is at least 90%, 95%, or 98% identical to SEQ ID NO: 80, where the HCVD includes a histidine at any of the specific combinations of two or more amino acid positions in SEQ ID NO: 80 listed in Table 3.

In some examples, the first ABD comprises an LCVD that is at least 90%, 95%, or 98% identical to SEQ ID NO. 81, wherein the LCVD includes a histidine at position 52 in SEQ ID NO: 81; and an HCVD that is at least 90%, 95%, or 98% identical to SEQ ID NO: 80, where the HCVD includes a histidine at any of the specific combinations of two or more amino acid positions in SEQ ID NO: 80 listed in Table 3.

In some examples, the first ABD comprises an LCVD that is at least 90%, 95%, or 98% identical to SEQ ID NO: 81, wherein the LCVD includes a histidine at position 53 in SEQ ID NO: 81; and an HCVD that is at least 90%, 95%, or 98% identical to SEQ ID NO: 80, where the HCVD includes a histidine at any of the specific combinations of two or more amino acid positions in SEQ ID NO: 80 listed in Table 3.

In some examples, the first ABD comprises an LCVD that is at least 90%, 95%, or 98% identical to SEQ ID NO: 81, wherein the LCVD includes a histidine at position 54 in SEQ ID NO: 81; and an HCVD that is at least 90%, 95%, or 98% identical to SEQ ID NO: 80, where the HCVD includes a histidine at any of the specific combinations of two or more amino acid positions in SEQ ID NO: 80 listed in Table 3.

In some examples, the first ABD comprises an LCVD that is at least 90%, 95%, or 98% identical to SEQ ID NO: 81, wherein the LCVD includes a histidine at position 89 in SEQ ID NO: 81; and an HCVD that is at least 90%, 95%, or 98% identical to SEQ ID NO: 80, where the HCVD includes a histidine at any of the specific combinations of two or more amino acid positions in SEQ ID NO: 80 listed in Table 3.

In some examples, the first ABD comprises an LCVD that is at least 90%, 95%, or 98% identical to SEQ ID NO: 81, wherein the LCVD includes a histidine at position 93 in SEQ ID NO: 81; and an HCVD that is at least 90%, 95%, or 98% identical to SEQ ID NO: 80, where the HCVD includes a histidine at any of the specific combinations of two or more amino acid positions in SEQ ID NO: 80 listed in Table 3.

In some examples, the first ABD comprises an LCVD that is at least 90%, 95%, or 98% identical to SEQ ID NO: 81, wherein the LCVD includes a histidine at position 94 in SEQ ID NO: 81; and an HCVD that is at least 90%, 95%, or 98% identical to SEQ ID NO: 80, where the HCVD includes a histidine at any of the specific combinations of two or more amino acid positions in SEQ ID NO: 80 listed in Table 3.

In some examples, the first ABD comprises an LCVD that is at least 90%, 95%, or 98% identical to SEQ ID NO. 81, wherein the LCVD includes a histidine at position 95 in SEQ ID NO: 81; and an HCVD that is at least 90%, 95%, or 98% identical to SEQ ID NO: 80, where the HCVD includes a histidine at any of the specific combinations of two or more amino acid positions in SEQ ID NO: 80 listed in Table 3.

In some examples, the first ABD comprises an HCVD that is at least 90%, 95%, or 98% identical to SEQ ID NO: 80, wherein the HCVD includes a histidine at any one of positions 29, 53, 55, 64, 65, 98, 101, 104, 105, 107, and 108 in SEQ ID NO: 80; and an LCVD that is at least 90%, 95%, or 98% identical to SEQ ID NO: 81, where the LCVD includes a histidine at any of the specific combinations of two or more amino acid positions in SEQ ID NO: 81 listed in Table 4.

In some examples, the first ABD comprises an HCVD of SEQ ID NO: 80, one of the sequences set forth in SEQ ID NOs: 88-128, one of the sequences set forth in SEQ ID NOs: 397-406; and/or the first ABD includes an LCVD of SEQ ID NO: 81, one of the sequences set forth in SEQ ID NOs: 129-155, or one of the sequences set forth in SEQ ID NOs: 407-426.

In some examples of the ABPCs, the first ABD includes an LCVD comprising the sequence of SEQ ID NO: 81, one of the sequences set forth in SEQ ID NOs: 129-155, or one of the sequences set forth in SEQ ID NOs: 407-426 and an HCVD comprising the sequences of SEQ ID NO: 80, one of the sequences set forth in SEQ ID NOs: 88-128, or one of the sequences set forth in SEQ ID NOs: 397-406.

In some examples, the first ABD includes an HCVD comprising the sequence of SEQ ID NO: 80, and an LCVD comprising the sequence of SEQ ID NO: 81, one of the sequences set forth in SEQ ID NOs: 129-155, or one of the sequences set forth in SEQ ID NOs: 407-426.

In some examples, the first ABD includes an HCVD comprising the sequence of SEQ ID NO: 88, and an LCVD comprising the sequence of SEQ ID NO: 81, one of the sequences set forth in SEQ ID NOs: 129-155, or one of the sequences set forth in SEQ ID NOs: 407-426.

In some examples, the first ABD includes an HCVD comprising the sequence of SEQ ID NO: 89, and an LCVD comprising the sequence of SEQ ID NO: 81, one of the sequences set forth in SEQ ID NOs: 129-155, or one of the sequences set forth in SEQ ID NOs: 407-426.

In some examples, the first ABD includes an HCVD comprising the sequence of SEQ ID NO: 90, and an LCVD comprising the sequence of SEQ ID NO: 81, one of the sequences set forth in SEQ ID NOs: 129-155, or one of the sequences set forth in SEQ ID NOs: 407-426.

In some examples, the first ABD includes an HCVD comprising the sequence of SEQ ID NO: 91, and an LCVD comprising the sequence of SEQ ID NO: 81, one of the sequences set forth in SEQ ID NOs: 129-155, or one of the sequences set forth in SEQ ID NOs: 407-426.

In some examples, the first ABD includes an HCVD comprising the sequence of SEQ ID NO: 92, and an LCVD comprising the sequence of SEQ ID NO: 81, one of the sequences set forth in SEQ ID NOs: 129-155, or one of the sequences set forth in SEQ ID NOs: 407-426.

In some examples, the first ABD includes an HCVD comprising the sequence of SEQ ID NO: 93, and an LCVD comprising the sequence of SEQ ID NO: 81, one of the sequences set forth in SEQ ID NOs: 129-155, or one of the sequences set forth in SEQ ID NOs: 407-426.

In some examples, the first ABD includes an HCVD comprising the sequence of SEQ ID NO: 94, and an LCVD comprising the sequence of SEQ ID NO: 81, one of the sequences set forth in SEQ ID NOs: 129-155, or one of the sequences set forth in SEQ ID NOs: 407-426.

In some examples, the first ABD includes an HCVD comprising the sequence of SEQ ID NO: 95, and an LCVD comprising the sequence of SEQ ID NO: 81, one of the sequences set forth in SEQ ID NOs: 129-155, or one of the sequences set forth in SEQ ID NOs: 407-426.

In some examples, the first ABD includes an HCVD comprising the sequence of SEQ ID NO: 96, and an LCVD comprising the sequence of SEQ ID NO: 81, one of the sequences set forth in SEQ ID NOs: 129-155, or one of the sequences set forth in SEQ ID NOs: 407-426.

In some examples, the first ABD includes an HCVD comprising the sequence of SEQ ID NO: 97, and an LCVD comprising the sequence of SEQ ID NO: 81, one of the sequences set forth in SEQ ID NOs: 129-155, or one of the sequences set forth in SEQ ID NOs: 407426.

In some examples, the first ABD includes an HCVD comprising the sequence of SEQ ID NO: 98, and an LCVD comprising the sequence of SEQ ID NO: 81, one of the sequences set forth in SEQ ID NOs: 129-155, or one of the sequences set forth in SEQ ID NOs: 407-426.

In some examples, the first ABD includes an HCVD comprising the sequence of SEQ ID NO: 99, and an LCVD comprising the sequence of SEQ ID NO: 81, one of the sequences set forth in SEQ ID NOs: 129-155, or one of the sequences set forth in SEQ ID NOs: 407426.

In some examples, the first ABD includes an HCVD comprising the sequence of SEQ ID NO: 100, and an LCVD comprising the sequence of SEQ ID NO: 81, one of the sequences set forth in SEQ ID NOs: 129-155, or one of the sequences set forth in SEQ ID NOs: 407426.

In some examples, the first ABD includes an HCVD comprising the sequence of SEQ ID NO: 101, and an LCVD comprising the sequence of SEQ ID NO: 81, one of the sequences set forth in SEQ ID NOs: 129-155, or one of the sequences set forth in SEQ ID NOs: 407-426.

In some examples, the first ABD includes an HCVD comprising the sequence of SEQ ID NO: 102, and an LCVD comprising the sequence of SEQ ID NO: 81, one of the sequences set forth in SEQ ID NOs: 129-155, or one of the sequences set forth in SEQ ID NOs: 407426.

In some examples, the first ABD includes an HCVD comprising the sequence of SEQ ID NO: 103, and an LCVD comprising the sequence of SEQ ID NO: 81, one of the sequences set forth in SEQ ID NOs: 129-155, or one of the sequences set forth in SEQ ID NOs: 407426.

In some examples, the first ABD includes an HCVD comprising the sequence of SEQ ID NO: 104, and an LCVD comprising the sequence of SEQ ID NO: 81, one of the sequences set forth in SEQ ID NOs: 129-155, or one of the sequences set forth in SEQ ID NOs: 407426.

In some examples, the first ABD includes an HCVD comprising the sequence of SEQ ID NO: 105, and an LCVD comprising the sequence of SEQ ID NO: 81, one of the sequences set forth in SEQ ID NOs: 129-155, or one of the sequences set forth in SEQ ID NOs: 407426.

In some examples, the first ABD includes an HCVD comprising the sequence of SEQ ID NO: 106, and an LCVD comprising the sequence of SEQ ID NO: 81, one of the sequences set forth in SEQ ID NOs: 129-155, or one of the sequences set forth in SEQ ID NOs: 407-426.

In some examples, the first ABD includes an HCVD comprising the sequence of SEQ ID NO: 107, and an LCVD comprising the sequence of SEQ ID NO: 81, one of the sequences set forth in SEQ ID NOs: 129-155, or one of the sequences set forth in SEQ ID NOs: 407-426.

In some examples, the first ABD includes an HCVD comprising the sequence of SEQ ID NO: 108, and an LCVD comprising the sequence of SEQ ID NO: 81, one of the sequences set forth in SEQ ID NOs: 129-155, or one of the sequences set forth in SEQ ID NOs: 407-426.

In some examples, the first ABD includes an HCVD comprising the sequence of SEQ ID NO: 109, and an LCVD comprising the sequence of SEQ ID NO: 81, one of the sequences set forth in SEQ ID NOs: 129-155, or one of the sequences set forth in SEQ ID NOs: 407-426.

In some examples, the first ABD includes an HCVD comprising the sequence of SEQ ID NO: 110, and an LCVD comprising the sequence of SEQ ID NO: 81, one of the sequences set forth in SEQ ID NOs: 129-155, or one of the sequences set forth in SEQ ID NOs: 407-426.

In some examples, the first ABD includes an HCVD comprising the sequence of SEQ ID NO: 111, and an LCVD comprising the sequence of SEQ ID NO: 81, one of the sequences set forth in SEQ ID NOs: 129-155, or one of the sequences set forth in SEQ ID NOs: 407-426.

In some examples, the first ABD includes an HCVD comprising the sequence of SEQ ID NO: 112, and an LCVD comprising the sequence of SEQ ID NO: 81, one of the sequences set forth in SEQ ID NOs: 129-155, or one of the sequences set forth in SEQ ID NOs: 407-426.

In some examples, the first ABD includes an HCVD comprising the sequence of SEQ ID NO: 113, and an LCVD comprising the sequence of SEQ ID NO: 81, one of the sequences set forth in SEQ ID NOs: 129-155, or one of the sequences set forth in SEQ ID NOs: 407-426.

In some examples, the first ABD includes an HCVD comprising the sequence of SEQ ID NO: 114, and an LCVD comprising the sequence of SEQ ID NO: 81, one of the sequences set forth in SEQ ID NOs: 129-155, or one of the sequences set forth in SEQ ID NOs: 407-426.

In some examples, the first ABD includes an HCVD comprising the sequence of SEQ ID NO: 115, and an LCVD comprising the sequence of SEQ ID NO: 81, one of the sequences set forth in SEQ ID NOs: 129-155, or one of the sequences set forth in SEQ ID NOs: 407-426.

In some examples, the first ABD includes an HCVD comprising the sequence of SEQ ID NO: 116, and an LCVD comprising the sequence of SEQ ID NO: 81, one of the sequences set forth in SEQ ID NOs: 129-155, or one of the sequences set forth in SEQ ID NOs: 407-426.

In some examples, the first ABD includes an HCVD comprising the sequence of SEQ ID NO: 117, and an LCVD comprising the sequence of SEQ ID NO: 81, one of the sequences set forth in SEQ ID NOs: 129-155, or one of the sequences set forth in SEQ ID NOs: 407-426.

In some examples, the first ABD includes an HCVD comprising the sequence of SEQ ID NO: 118, and an LCVD comprising the sequence of SEQ ID NO: 81, one of the sequences set forth in SEQ ID NOs: 129-155, or one of the sequences set forth in SEQ ID NOs: 407-426.

In some examples, the first ABD includes an HCVD comprising the sequence of SEQ ID NO: 119, and an LCVD comprising the sequence of SEQ ID NO: 81, one of the sequences set forth in SEQ ID NOs: 129-155, or one of the sequences set forth in SEQ ID NOs: 407-426.

In some examples, the first ABD includes an HCVD comprising the sequence of SEQ ID NO: 120, and an LCVD comprising the sequence of SEQ ID NO: 81, one of the sequences set forth in SEQ ID NOs: 129-155, or one of the sequences set forth in SEQ ID NOs: 407-426.

In some examples, the first ABD includes an HCVD comprising the sequence of SEQ ID NO: 121, and an LCVD comprising the sequence of SEQ ID NO: 81, one of the sequences set forth in SEQ ID NOs: 129-155, or one of the sequences set forth in SEQ ID NOs: 407-426.

In some examples, the first ABD includes an HCVD comprising the sequence of SEQ ID NO: 122, and an LCVD comprising the sequence of SEQ ID NO: 81, one of the sequences set forth in SEQ ID NOs: 129-155, or one of the sequences set forth in SEQ ID NOs: 407-426.

In some examples, the first ABD includes an HCVD comprising the sequence of SEQ ID NO: 123, and an LCVD comprising the sequence of SEQ ID NO: 81, one of the sequences set forth in SEQ ID NOs: 129-155, or one of the sequences set forth in SEQ ID NOs: 407-426.

In some examples, the first ABD includes an HCVD comprising the sequence of SEQ ID NO: 124, and an LCVD comprising the sequence of SEQ ID NO: 81, one of the sequences set forth in SEQ ID NOs: 129-155, or one of the sequences set forth in SEQ ID NOs: 407-426.

In some examples, the first ABD includes an HCVD comprising the sequence of SEQ ID NO: 125, and an LCVD comprising the sequence of SEQ ID NO: 81, one of the sequences set forth in SEQ ID NOs: 129-155, or one of the sequences set forth in SEQ ID NOs: 407-426.

In some examples, the first ABD includes an HCVD comprising the sequence of SEQ ID NO: 126, and an LCVD comprising the sequence of SEQ ID NO: 81, one of the sequences set forth in SEQ ID NOs: 129-155, or one of the sequences set forth in SEQ ID NOs: 407-426.

In some examples, the first ABD includes an HCVD comprising the sequence of SEQ ID NO: 127, and an LCVD comprising the sequence of SEQ ID NO: 81, one of the sequences set forth in SEQ ID NOs: 129-155, or one of the sequences set forth in SEQ ID NOs: 407-426.

In some examples, the first ABD includes an HCVD comprising the sequence of SEQ ID NO: 128, and an LCVD comprising the sequence of SEQ ID NO: 81, one of the sequences set forth in SEQ ID NOs: 129-155, or one of the sequences set forth in SEQ ID NOs: 407-426.

In some examples, the first ABD includes an HCVD comprising the sequence of SEQ ID NO: 397, and an LCVD comprising the sequence of SEQ ID NO: 81, one of the sequences set forth in SEQ ID NOs: 129-155, or one of the sequences set forth in SEQ ID NOs: 407-426.

In some examples, the first ABD includes an HCVD comprising the sequence of SEQ ID NO: 398, and an LCVD comprising the sequence of SEQ ID NO: 81, one of the sequences set forth in SEQ ID NOs: 129-155, or one of the sequences set forth in SEQ ID NOs: 407-426.

In some examples, the first ABD includes an HCVD comprising the sequence of SEQ ID NO: 399, and an LCVD comprising the sequence of SEQ ID NO: 81, one of the sequences set forth in SEQ ID NOs: 129-155, or one of the sequences set forth in SEQ ID NOs: 407-426.

In some examples, the first ABD includes an HCVD comprising the sequence of SEQ ID NO: 400, and an LCVD comprising the sequence of SEQ ID NO: 81, one of the sequences set forth in SEQ ID NOs: 129-155, or one of the sequences set forth in SEQ ID NOs: 407-426.

In some examples, the first ABD includes an HCVD comprising the sequence of SEQ ID NO: 401, and an LCVD comprising the sequence of SEQ ID NO: 81, one of the sequences set forth in SEQ ID NOs: 129-155, or one of the sequences set forth in SEQ ID NOs: 407-426.

In some examples, the first ABD includes an HCVD comprising the sequence of SEQ ID NO: 402, and an LCVD comprising the sequence of SEQ ID NO: 81, one of the sequences set forth in SEQ ID NOs: 129-155, or one of the sequences set forth in SEQ ID NOs: 407-426.

In some examples, the first ABD includes an HCVD comprising the sequence of SEQ ID NO: 403, and an LCVD comprising the sequence of SEQ ID NO: 81, one of the sequences set forth in SEQ ID NOs: 129-155, or one of the sequences set forth in SEQ ID NOs: 407-426.

In some examples, the first ABD includes an HCVD comprising the sequence of SEQ ID NO: 404, and an LCVD comprising the sequence of SEQ ID NO: 81, one of the sequences set forth in SEQ ID NOs: 129-155, or one of the sequences set forth in SEQ ID NOs: 407-426.

In some examples, the first ABD includes an HCVD comprising the sequence of SEQ ID NO: 405, and an LCVD comprising the sequence of SEQ ID NO: 81, one of the sequences set forth in SEQ ID NOs: 129-155, or one of the sequences set forth in SEQ ID NOs: 407-426.

In some examples, the first ABD includes an HCVD comprising the sequence of SEQ ID NO: 406, and an LCVD comprising the sequence of SEQ ID NO: 81, one of the sequences set forth in SEQ ID NOs: 129-155, or one of the sequences set forth in SEQ ID NOs: 407-426.

In some examples, the first ABD includes an LCVD comprising the sequence of SEQ ID NO: 81, and an HCVD comprising the sequence of SEQ ID NO: 80, one of the sequences set forth in SEQ ID NOs: 88-128, or one of the sequences set forth in SEQ ID NOs: 397-406.

In some examples, the first ABD includes an LCVD comprising the sequence of SEQ ID NO: 129, and an HCVD comprising the sequence of SEQ ID NO: 80, one of the sequences set forth in SEQ ID NOs: 88-128, or one of the sequences set forth in SEQ ID NOs: 397-406.

In some examples, the first ABD includes an LCVD comprising the sequence of SEQ ID NO: 130, and an HCVD comprising the sequence of SEQ ID NO: 80, one of the sequences set forth in SEQ ID NOs: 88-128, or one of the sequences set forth in SEQ ID NOs: 397-406.

In some examples, the first ABD includes an LCVD comprising the sequence of SEQ ID NO: 131, and an HCVD comprising the sequence of SEQ ID NO: 80, one of the sequences set forth in SEQ ID NOs: 88-128, or one of the sequences set forth in SEQ ID NOs: 397-406.

In some examples, the first ABD includes an LCVD comprising the sequence of SEQ ID NO: 132, and an HCVD comprising the sequence of SEQ ID NO: 80, one of the sequences set forth in SEQ ID NOs: 88-128, or one of the sequences set forth in SEQ ID NOs: 397-406.

In some examples, the first ABD includes an LCVD comprising the sequence of SEQ ID NO: 133, and an HCVD comprising the sequence of SEQ ID NO: 80, one of the sequences set forth in SEQ ID NOs: 88-128, or one of the sequences set forth in SEQ ID NOs: 397-406.

In some examples, the first ABD includes an LCVD comprising the sequence of SEQ ID NO: 134, and an HCVD comprising the sequence of SEQ ID NO: 80, one of the sequences set forth in SEQ ID NOs: 88-128, or one of the sequences set forth in SEQ ID NOs: 397-406.

In some examples, the first ABD includes an LCVD comprising the sequence of SEQ ID NO: 135, and an HCVD comprising the sequence of SEQ ID NO: 80, one of the sequences set forth in SEQ ID NOs: 88-128, or one of the sequences set forth in SEQ ID NOs: 397-406.

In some examples, the first ABD includes an LCVD comprising the sequence of SEQ ID NO: 136, and an HCVD comprising the sequence of SEQ ID NO: 80, one of the sequences set forth in SEQ ID NOs: 88-128, or one of the sequences set forth in SEQ ID NOs: 397-406.

In some examples, the first ABD includes an LCVD comprising the sequence of SEQ ID NO: 137, and an HCVD comprising the sequence of SEQ ID NO: 80, one of the sequences set forth in SEQ ID NOs: 88-128, or one of the sequences set forth in SEQ ID NOs: 397-406.

In some examples, the first ABD includes an LCVD comprising the sequence of SEQ ID NO: 138, and an HCVD comprising the sequence of SEQ ID NO: 80, one of the sequences set forth in SEQ ID NOs: 88-128, or one of the sequences set forth in SEQ ID NOs: 397-406.

In some examples, the first ABD includes an LCVD comprising the sequence of SEQ ID NO: 139, and an HCVD comprising the sequence of SEQ ID NO: 80, one of the sequences set forth in SEQ ID NOs: 88-128, or one of the sequences set forth in SEQ ID NOs: 397-406.

In some examples, the first ABD includes an LCVD comprising the sequence of SEQ ID NO: 140, and an HCVD comprising the sequence of SEQ ID NO: 80, one of the sequences set forth in SEQ ID NOs: 88-128, or one of the sequences set forth in SEQ ID NOs: 397-406.

In some examples, the first ABD includes an LCVD comprising the sequence of SEQ ID NO: 141, and an HCVD comprising the sequence of SEQ ID NO: 80, one of the sequences set forth in SEQ ID NOs: 88-128, or one of the sequences set forth in SEQ ID NOs: 397-406.

In some examples, the first ABD includes an LCVD comprising the sequence of SEQ ID NO: 142, and an HCVD comprising the sequence of SEQ ID NO: 80, one of the sequences set forth in SEQ ID NOs: 88-128, or one of the sequences set forth in SEQ ID NOs: 397-406.

In some examples, the first ABD includes an LCVD comprising the sequence of SEQ ID NO: 143, and an HCVD comprising the sequence of SEQ ID NO: 80, one of the sequences set forth in SEQ ID NOs: 88-128, or one of the sequences set forth in SEQ ID NOs: 397-406.

In some examples, the first ABD includes an LCVD comprising the sequence of SEQ ID NO: 144, and an HCVD comprising the sequence of SEQ ID NO: 80, one of the sequences set forth in SEQ ID NOs: 88-128, or one of the sequences set forth in SEQ ID NOs: 397-406.

In some examples, the first ABD includes an LCVD comprising the sequence of SEQ ID NO: 145, and an HCVD comprising the sequence of SEQ ID NO: 80, one of the sequences set forth in SEQ ID NOs: 88-128, or one of the sequences set forth in SEQ ID NOs: 397-406.

In some examples, the first ABD includes an LCVD comprising the sequence of SEQ ID NO: 146, and an HCVD comprising the sequence of SEQ ID NO: 80, one of the sequences set forth in SEQ ID NOs: 88-128, or one of the sequences set forth in SEQ ID NOs: 397-406.

In some examples, the first ABD includes an LCVD comprising the sequence of SEQ ID NO: 147, and an HCVD comprising the sequence of SEQ ID NO: 80, one of the sequences set forth in SEQ ID NOs: 88-128, or one of the sequences set forth in SEQ ID NOs: 397-406.

In some examples, the first ABD includes an LCVD comprising the sequence of SEQ ID NO: 148, and an HCVD comprising the sequence of SEQ ID NO: 80, one of the sequences set forth in SEQ ID NOs: 88-128, or one of the sequences set forth in SEQ ID NOs: 397-406.

In some examples, the first ABD includes an LCVD comprising the sequence of SEQ ID NO: 149, and an HCVD comprising the sequence of SEQ ID NO: 80, one of the sequences set forth in SEQ ID NOs: 88-128, or one of the sequences set forth in SEQ ID NOs: 397-406.

In some examples, the first ABD includes an LCVD comprising the sequence of SEQ ID NO: 150, and an HCVD comprising the sequence of SEQ ID NO: 80, one of the sequences set forth in SEQ ID NOs: 88-128, or one of the sequences set forth in SEQ ID NOs: 397-406.

In some examples, the first ABD includes an LCVD comprising the sequence of SEQ ID NO: 151, and an HCVD comprising the sequence of SEQ ID NO: 80, one of the sequences set forth in SEQ ID NOs: 88-128, or one of the sequences set forth in SEQ ID NOs: 397-406.

In some examples, the first ABD includes an LCVD comprising the sequence of SEQ ID NO: 152, and an HCVD comprising the sequence of SEQ ID NO: 80, one of the sequences set forth in SEQ ID NOs: 88-128, or one of the sequences set forth in SEQ ID NOs: 397-406.

In some examples, the first ABD includes an LCVD comprising the sequence of SEQ ID NO: 153, and an HCVD comprising the sequence of SEQ ID NO: 80, one of the sequences set forth in SEQ ID NOs: 88-128, or one of the sequences set forth in SEQ ID NOs: 397-406.

In some examples, the first ABD includes an LCVD comprising the sequence of SEQ ID NO: 154, and an HCVD comprising the sequence of SEQ ID NO: 80, one of the sequences set forth in SEQ ID NOs: 88-128, or one of the sequences set forth in SEQ ID NOs: 397-406.

In some examples, the first ABD includes an LCVD comprising the sequence of SEQ ID NO: 155, and an HCVD comprising the sequence of SEQ ID NO: 80, one of the sequences set forth in SEQ ID NOs: 88-128, or one of the sequences set forth in SEQ ID NOs: 397-406.

In some examples, the first ABD includes an LCVD comprising the sequence of SEQ ID NO: 407, and an HCVD comprising the sequence of SEQ ID NO: 80, one of the sequences set forth in SEQ ID NOs: 88-128, or one of the sequences set forth in SEQ ID NOs: 397-406.

In some examples, the first ABD includes an LCVD comprising the sequence of SEQ ID NO: 408, and an HCVD comprising the sequence of SEQ ID NO: 80, one of the sequences set forth in SEQ ID NOs: 88-128, or one of the sequences set forth in SEQ ID NOs: 397-406.

In some examples, the first ABD includes an LCVD comprising the sequence of SEQ ID NO: 409, and an HCVD comprising the sequence of SEQ ID NO: 80, one of the sequences set forth in SEQ ID NOs: 88-128, or one of the sequences set forth in SEQ ID NOs: 397-406.

In some examples, the first ABD includes an LCVD comprising the sequence of SEQ ID NO: 410, and an HCVD comprising the sequence of SEQ ID NO: 80, one of the sequences set forth in SEQ ID NOs: 88-128, or one of the sequences set forth in SEQ ID NOs: 397-406.

In some examples, the first ABD includes an LCVD comprising the sequence of SEQ ID NO: 411, and an HCVD comprising the sequence of SEQ ID NO: 80, one of the sequences set forth in SEQ ID NOs: 88-128, or one of the sequences set forth in SEQ ID NOs: 397-406.

In some examples, the first ABD includes an LCVD comprising the sequence of SEQ ID NO: 412, and an HCVD comprising the sequence of SEQ ID NO: 80, one of the sequences set forth in SEQ ID NOs: 88-128, or one of the sequences set forth in SEQ ID NOs: 397-406.

In some examples, the first ABD includes an LCVD comprising the sequence of SEQ ID NO: 413, and an HCVD comprising the sequence of SEQ ID NO: 80, one of the sequences set forth in SEQ ID NOs: 88-128, or one of the sequences set forth in SEQ ID NOs: 397-406.

In some examples, the first ABD includes an LCVD comprising the sequence of SEQ ID NO: 414, and an HCVD comprising the sequence of SEQ ID NO: 80, one of the sequences set forth in SEQ ID NOs: 88-128, or one of the sequences set forth in SEQ ID NOs: 397-406.

In some examples, the first ABD includes an LCVD comprising the sequence of SEQ ID NO: 415, and an HCVD comprising the sequence of SEQ ID NO: 80, one of the sequences set forth in SEQ ID NOs: 88-128, or one of the sequences set forth in SEQ ID NOs: 397-406.

In some examples, the first ABD includes an LCVD comprising the sequence of SEQ ID NO: 416, and an HCVD comprising the sequence of SEQ ID NO: 80, one of the sequences set forth in SEQ ID NOs: 88-128, or one of the sequences set forth in SEQ ID NOs: 397-406.

In some examples, the first ABD includes an LCVD comprising the sequence of SEQ ID NO: 417, and an HCVD comprising the sequence of SEQ ID NO: 80, one of the sequences set forth in SEQ ID NOs: 88-128, or one of the sequences set forth in SEQ ID NOs: 397-406.

In some examples, the first ABD includes an LCVD comprising the sequence of SEQ ID NO: 418, and an HCVD comprising the sequence of SEQ ID NO: 80, one of the sequences set forth in SEQ ID NOs: 88-128, or one of the sequences set forth in SEQ ID NOs: 397-406.

In some examples, the first ABD includes an LCVD comprising the sequence of SEQ ID NO: 419, and an HCVD comprising the sequence of SEQ ID NO: 80, one of the sequences set forth in SEQ ID NOs: 88-128, or one of the sequences set forth in SEQ ID NOs: 397-406.

In some examples, the first ABD includes an LCVD comprising the sequence of SEQ ID NO: 420, and an HCVD comprising the sequence of SEQ ID NO: 80, one of the sequences set forth in SEQ ID NOs: 88-128, or one of the sequences set forth in SEQ ID NOs: 397-406.

In some examples, the first ABD includes an LCVD comprising the sequence of SEQ ID NO: 421, and an HCVD comprising the sequence of SEQ ID NO: 80, one of the sequences set forth in SEQ ID NOs: 88-128, or one of the sequences set forth in SEQ ID NOs: 397-406.

In some examples, the first ABD includes an LCVD comprising the sequence of SEQ ID NO: 422, and an HCVD comprising the sequence of SEQ ID NO: 80, one of the sequences set forth in SEQ ID NOs: 88-128, or one of the sequences set forth in SEQ ID NOs: 397-406.

In some examples, the first ABD includes an LCVD comprising the sequence of SEQ ID NO: 423, and an HCVD comprising the sequence of SEQ ID NO: 80, one of the sequences set forth in SEQ ID NOs: 88-128, or one of the sequences set forth in SEQ ID NOs: 397-406.

In some examples, the first ABD includes an LCVD comprising the sequence of SEQ ID NO: 424, and an HCVD comprising the sequence of SEQ ID NO: 80, one of the sequences set forth in SEQ ID NOs: 88-128, or one of the sequences set forth in SEQ ID NOs: 397-406.

In some examples, the first ABD includes an LCVD comprising the sequence of SEQ ID NO: 425, and an HCVD comprising the sequence of SEQ ID NO: 80, one of the sequences set forth in SEQ ID NOs: 88-128, or one of the sequences set forth in SEQ ID NOs: 397-406.

In some examples, the first ABD includes an LCVD comprising the sequence of SEQ ID NO: 426, and an HCVD comprising the sequence of SEQ ID NO: 80, one of the sequences set forth in SEQ ID NOs: 88-128, or one of the sequences set forth in SEQ ID NOs: 397-406.

In some examples, the first ABD includes an HCVD of hu7E6SVG with one or more amino acids substituted with a histidine. In some examples, the first ABD includes an LCVD of hu7E6SVG with one or more amino acids substituted with a histidine. In some examples, the first ABD includes an HCVD of hu7E6SVG with one or more amino acids substituted with a histidine; and an LCVD of hu7E6SVG with one or more amino acids substituted with a histidine. In some examples, the HCVD of hu7E6SVG comprises SEQ ID NO: 156. In some examples, the LCVD of hu7E6SVG comprises SEQ ID NO. 157. In each of the foregoing examples, an alanine may be substituted where a histidine was already present.

In some examples, the first ABD comprises an HCVD comprising a CDR1, a CDR2, and a CDR3 of SEQ ID NOs: 158, 159, and 160, respectively, with collectively a total of one or more amino acid positions in SEQ ID NOs: 158, 159, and 160 substituted with a histidine. In some examples, the first ABD comprises an LCVD comprising a CDR1, a CDR2, and a CDR3 of SEQ ID NOs: 161, 162, and 163, respectively, with collectively a total of one or more amino acid positions in SEQ ID NOs: 161, 162, and 163 substituted with a histidine. In some examples, the first ABD includes: an HCVD comprising a CDR1, a CDR2, and a CDR3 of SEQ ID NOs: 158, 159, and 160, respectively, with collectively a total of one or more amino acid positions in NOs: 158, 159, and 160 substituted with a histidine; and an LCVD comprising a CDR1, a CDR2, and a CDR3 of SEQ ID NOs: 161, 162, and 163, respectively, with collectively a total of one or more amino acid positions in SEQ ID NOs: 161, 162, and 163 substituted with a histidine. In each of the foregoing examples, an alanine may be substituted where a histidine was already present.

In some examples, the first ABD includes an HCVD that is at least 90%, 95%, or 98% identical to SEQ ID NO: 156, where the HCVD includes a histidine at one or more amino acid positions in SEQ ID NO: 156 selected from: 26, 29, 30, 33, 34, 51, 54, 56, 57, 59, 102, 103, and 106. In some examples, the first ABD includes an LCVD that is at least 90%, 95%, or 98% identical to SEQ ID NO: 157, where the LCVD includes a histidine at one or more amino acid positions in SEQ ID NO: 157 selected from: 25, 32, 34, 36, 54, 56, 57, 58, 59, 60, 93, 96, 97, 99, 100, and 101. In some examples, the first ABD includes an HCVD that is at least 90%, 95%, or 98% identical to SEQ ID NO: 156, where the HCVD includes a histidine at one or more amino acid positions in SEQ ID NO. 156 selected from: 26, 29, 30, 33, 34, 51, 54, 56, 57, 59, 102, 103, and 106, and an LCVD that is at least 90%, 95%, or 98% identical to SEQ ID NO: 157, where the LCVD includes a histidine at one or more amino acid positions in SEQ ID NO: 157 selected from 25, 32, 34, 36, 54, 56, 57, 58, 59, 60, 93, 96, 97, 99, 100, and 101.

TABLE 5
Exemplary Combinations of Amino Acid Positions in SEQ ID NO: 156 that can be Substituted

| | Col | | | | | | | | | | | | | |
|---|---|---|---|---|---|---|---|---|---|---|---|---|---|---|
| | 1 | 2 | 3 | 4 | 5 | 6 | 7 | 8 | 9 | 10 | 11 | 12 | 13 | 14 |
| Pos | 26 | 29 | 30 | 33 | 34 | 51 | 54 | 56 | 57 | 59 | 102 | 103 | 106 | 35 (A) |

Exemplary 2, 3, and 4 AA position combinations that can be substituted with histidine (or an alanine at
position 35 of SEQ ID NO: 156)

| | |
|---|---|
| A | Each and every unique 2-position combination of X & Y, wherein X and Y are each selected |
| | (without replacement) from a SEQ ID NO: 156 AA position indicated in columns 1-14. |
| | Expressed another way, the combinations of positions recited in the following combinations of |
| | columns: 1 + 2, 1 + 3, 1 + 4, 1 + 5, 1 + 6, 1 + 7, 1 + 8, 1 + 9, 1 + 10, 1 + 11, 1 + 12, 1 + 13, 1 + 14, 2 + 3, 2 + 4, |
| | 2 + 5, 2 + 6, 2 + 7, 2 + 8, 2 + 9, 2 + 10, 2 + 11, 2 + 12, 2 + 13, 2 + 14, 3 + 4, 3 + 5, 3 + 6, 3 + 7, 3 + 8, 3 + 9, 3 + 10, |
| | 3 + 11, 3 + 12, 3 + 13, 3 + 14, 4 + 5, 4 + 6, 4 + 7, 4 + 8, 4 + 9, 4 + 10, 4 + 11, 4 + 12, 4 + 13, 4 + 14, 5 + 6, 5 + 7, |
| | 5 + 8, 5 + 9, 5 + 10, 5 + 11, 5 + 12, 5 + 13, 5 + 14, 6 + 7, 6 + 8, 6 + 9, 6 + 10, 6 + 11, 6 + 12, 6 + 13, 6 + 14, 7 + 8, |
| | 7 + 9, 7 + 10, 7 + 11, 7 + 12, 7 + 13, 7 + 14, 8 + 9, 8 + 10, 8 + 11, 8 + 12, 8 + 13, 8 + 14, 9 + 10, 9 + 11, 9 + 12, |
| | 9 + 13, 9 + 14, 10 + 11, 10 + 12, 10 + 13, 10 + 14, 11 + 12, 11 + 13, 11 + 14, 12 + 13, 12 + 14, and 13 + 14. |
| B | Each and every unique 3-position combination of X & Y & Z, wherein X, Y, and Z are each |
| | selected (without replacement) from a SEQ ID NO: 156 AA position indicated in columns 1-14. |
| | Expressed another way, the combinations of positions recited in the following combinations of |
| | columns: 1 + 2 + 3, 1 + 2 + 4, 1 + 2 + 5, 1 + 2 + 6, 1 + 2 + 7, 1 + 2 + 8, 1 + 2 + 9, 1 + 2 + 10, 1 + 2 + 11, 1 + 2 + 12, |
| | 1 + 2 + 13, 1 + 2 + 14, 1 + 3 + 4, 1 + 3 + 5, 1 + 3 + 6, 1 + 3 + 7, 1 + 3 + 8, 1 + 3 + 9, 1 + 3 + 10, 1 + 3 + 11, 1 + 3 + 12, |
| | 1 + 3 + 13, 1 + 3 + 14, 1 + 4 + 5, 1 + 4 + 6, 1 + 4 + 7, 1 + 4 + 8, 1 + 4 + 9, 1 + 4 + 10, 1 + 4 + 11, 1 + 4 + 12, 1 + 4 + 13, |
| | 1 + 4 + 14, 1 + 5 + 6, 1 + 5 + 7, 1 + 5 + 8, 1 + 5 + 9, 1 + 5 + 10, 1 + 5 + 11, 1 + 5 + 12, 1 + 5 + 13, 1 + 5 + 14, 1 + 6 + 7, |
| | 1 + 6 + 8, 1 + 6 + 9, 1 + 6 + 10, 1 + 6 + 11, 1 + 6 + 12, 1 + 6 + 13, 1 + 6 + 14, 1 + 7 + 8, 1 + 7 + 9, 1 + 7 + 10, 1 + 7 + 11, |
| | 1 + 7 + 12, 1 + 7 + 13, 1 + 7 + 14, 1 + 8 + 9, 1 + 8 + 10, 1 + 8 + 11, 1 + 8 + 12, 1 + 8 + 13, 1 + 8 + 14, 1 + 9 + 10, 1 + 9 + 11, |
| | 1 + 9 + 12, 1 + 9 + 13, 1 + 9 + 14, 1 + 10 + 11, 1 + 10 + 12, 1 + 10 + 13, 1 + 10 + 14, 1 + 11 + 12, 1 + 11 + 13, |
| | 1 + 11 + 14, 1 + 12 + 13, 1 + 12 + 14, 1 + 13 + 14, 2 + 3 + 4, 2 + 3 + 5, 2 + 3 + 6, 2 + 3 + 7, 2 + 3 + 8, 2 + 3 + 9, 2 + 3 + 10, |
| | 2 + 3 + 11, 2 + 3 + 12, 2 + 3 + 13, 2 + 3 + 14, 2 + 4 + 5, 2 + 4 + 6, 2 + 4 + 7, 2 + 4 + 8, 2 + 4 + 9, 2 + 4 + 10, 2 + 4 + 11, |
| | 2 + 4 + 12, 2 + 4 + 13, 2 + 4 + 14, 2 + 5 + 6, 2 + 5 + 7, 2 + 5 + 8, 2 + 5 + 9, 2 + 5 + 10, 2 + 5 + 11, 2 + 5 + 12, 2 + 5 + 13, |
| | 2 + 5 + 14, 2 + 6 + 7, 2 + 6 + 8, 2 + 6 + 9, 2 + 6 + 10, 2 + 6 + 11, 2 + 6 + 12, 2 + 6 + 13, 2 + 6 + 14, 2 + 7 + 8, 2 + 7 + 9, |
| | 2 + 7 + 10, 2 + 7 + 11, 2 + 7 + 12, 2 + 7 + 13, 2 + 7 + 14, 2 + 8 + 9, 2 + 8 + 10, 2 + 8 + 11, 2 + 8 + 12, 2 + 8 + 13, 2 + 8 + 14, |
| | 2 + 9 + 10, 2 + 9 + 11, 2 + 9 + 12, 2 + 9 + 13, 2 + 9 + 14, 2 + 10 + 11, 2 + 10 + 12, 2 + 10 + 13, 2 + 10 + 14, 2 + 11 + 12, |
| | 2 + 11 + 13, 2 + 11 + 14, 2 + 12 + 13, 2 + 12 + 14, 2 + 13 + 14, 3 + 4 + 5, 3 + 4 + 6, 3 + 4 + 7, 3 + 4 + 8, 3 + 4 + 9, |
| | 3 + 4 + 10, 3 + 4 + 11, 3 + 4 + 12, 3 + 4 + 13, 3 + 4 + 14, 3 + 5 + 6, 3 + 5 + 7, 3 + 5 + 8, 3 + 5 + 9, 3 + 5 + 10, 3 + 5 + 11, |
| | 3 + 5 + 12, 3 + 5 + 13, 3 + 5 + 14, 3 + 6 + 7, 3 + 6 + 8, 3 + 6 + 9, 3 + 6 + 10, 3 + 6 + 11, 3 + 6 + 12, 3 + 6 + 13, 3 + 6 + 14, |
| | 3 + 7 + 8, 3 + 7 + 9, 3 + 7 + 10, 3 + 7 + 11, 3 + 7 + 12, 3 + 7 + 13, 3 + 7 + 14, 3 + 8 + 9, 3 + 8 + 10, 3 + 8 + 11, 3 + 8 + 12, |
| | 3 + 8 + 13, 3 + 8 + 14, 3 + 9 + 10, 3 + 9 + 11, 3 + 9 + 12, 3 + 9 + 13, 3 + 9 + 14, 3 + 10 + 11, 3 + 10 + 12, 3 + 10 + 13, |
| | 3 + 10 + 14, 3 + 11 + 12, 3 + 11 + 13, 3 + 11 + 14, 3 + 12 + 13, 3 + 12 + 14, 3 + 13 + 14, 4 + 5 + 6, 4 + 5 + 7, 4 + 5 + 8, |
| | 4 + 5 + 9, 4 + 5 + 10, 4 + 5 + 11, 4 + 5 + 12, 4 + 5 + 13, 4 + 5 + 14, 4 + 6 + 7, 4 + 6 + 8, 4 + 6 + 9, 4 + 6 + 10, 4 + 6 + 11, |
| | 4 + 6 + 12, 4 + 6 + 13, 4 + 6 + 14, 4 + 7 + 8, 4 + 7 + 9, 4 + 7 + 10, 4 + 7 + 11, 4 + 7 + 12, 4 + 7 + 13, 4 + 7 + 14, 4 + 8 + 9, |
| | 4 + 8 + 10, 4 + 8 + 11, 4 + 8 + 12, 4 + 8 + 13, 4 + 8 + 14, 4 + 9 + 10, 4 + 9 + 11, 4 + 9 + 12, 4 + 9 + 13, 4 + 9 + 14, |
| | 4 + 10 + 11, 4 + 10 + 12, 4 + 10 + 13, 4 + 10 + 14, 4 + 11 + 12, 4 + 11 + 13, 4 + 11 + 14, 4 + 12 + 13, 4 + 12 + 14, |
| | 4 + 13 + 14, 5 + 6 + 7, 5 + 6 + 8, 5 + 6 + 9, 5 + 6 + 10, 5 + 6 + 11, 5 + 6 + 12, 5 + 6 + 13, 5 + 6 + 14, 5 + 7 + 8, 5 + 7 + 9, |
| | 5 + 7 + 10, 5 + 7 + 11, 5 + 7 + 12, 5 + 7 + 13, 5 + 7 + 14, 5 + 8 + 9, 5 + 8 + 10, 5 + 8 + 11, 5 + 8 + 12, 5 + 8 + 13, 5 + 8 + 14, |
| | 5 + 9 + 10, 5 + 9 + 11, 5 + 9 + 12, 5 + 9 + 13, 5 + 9 + 14, 5 + 10 + 11, 5 + 10 + 12, 5 + 10 + 13, 5 + 10 + 14, 5 + 11 + 12, |
| | 5 + 11 + 13, 5 + 11 + 14, 5 + 12 + 13, 5 + 12 + 14, 5 + 13 + 14, 6 + 7 + 8, 6 + 7 + 9, 6 + 7 + 10, 6 + 7 + 11, 6 + 7 + 12, |
| | 6 + 7 + 13, 6 + 7 + 14, 6 + 8 + 9, 6 + 8 + 10, 6 + 8 + 11, 6 + 8 + 12, 6 + 8 + 13, 6 + 8 + 14, 6 + 9 + 10, 6 + 9 + 11, 6 + 9 + 12, |
| | 6 + 9 + 13, 6 + 9 + 14, 6 + 10 + 11, 6 + 10 + 12, 6 + 10 + 13, 6 + 10 + 14, 6 + 11 + 12, 6 + 11 + 13, 6 + 11 + 14, |
| | 6 + 12 + 13, 6 + 12 + 14, 6 + 13 + 14, 7 + 8 + 9, 7 + 8 + 10, 7 + 8 + 11, 7 + 8 + 12, 7 + 8 + 13, 7 + 8 + 14, 7 + 9 + 10, |
| | 7 + 9 + 11, 7 + 9 + 12, 7 + 9 + 13, 7 + 9 + 14, 7 + 10 + 11, 7 + 10 + 12, 7 + 10 + 13, 7 + 10 + 14, 7 + 11 + 12, 7 + 11 + 13, |
| | 7 + 11 + 14, 7 + 12 + 13, 7 + 12 + 14, 7 + 13 + 14, 8 + 9 + 10, 8 + 9 + 11, 8 + 9 + 12, 8 + 9 + 13, 8 + 9 + 14, 8 + 10 + 11, |
| | 8 + 10 + 12, 8 + 10 + 13, 8 + 10 + 14, 8 + 11 + 12, 8 + 11 + 13, 8 + 11 + 14, 8 + 12 + 13, 8 + 12 + 14, 8 + 13 + 14, |
| | 9 + 10 + 11, 9 + 10 + 12, 9 + 10 + 13, 9 + 10 + 14, 9 + 11 + 12, 9 + 11 + 13, 9 + 11 + 14, 9 + 12 + 13, 9 + 12 + 14, |
| | 9 + 13 + 14, 10 + 11 + 12, 10 + 11 + 13, 10 + 11 + 14, 10 + 12 + 13, 10 + 12 + 14, 10 + 13 + 14, 11 + 12 + 13, |
| | 11 + 12 + 14, 11 + 13 + 14, and 12 + 13 + 14. |
| C | Each and every unique 4-position combination of X & Y & Z & α, wherein X, Y, and Z are |
| | together one of the 3-position combinations recited in “B” (above), and α is selected from a SEQ |
| | ID NO: 156 amino acid position recited in one of columns 1 to 14, without replacement (i.e. |
| | X ≠ Y ≠ Z ≠ α). |

In some examples, an HCVD includes an HCVD that is at least 90%, 95%, or 98% identical to SEQ ID NO: 156, where the HCVD includes a histidine at any of the specific combinations of two or more amino acid positions in SEQ ID NO: 156 listed in Table 5.

In some examples, an LCVD includes an LCVD that is at least 90%, 95%, or 98% identical to SEQ ID NO: 157, where the LCVD includes a histidine at any of the specific combinations of two or more amino acid positions in SEQ ID NO: 157 listed in Table 6.

In some examples, the first ABD includes an HCVD that is at least 90% (e.g., at least 92%, 94%, 96%, 98%, at least 99%, or 100%) identical to SEQ ID NO: 156, where the HCVD includes an alanine at position 35 in SEQ ID NO: 156.

In some examples the first ABD includes: an HCVD that is at least 90° % (e.g., at least 92%, 94%, 96%, 98%, at least 99%, or 100/c) identical to SEQ ID NO: 156, where the HCVD includes an alanine at position 35 in SEQ ID NO: 156, and an LCVD that is at least 90% (e.g., at least 92%, 94%, 96%, 98%, at least 99%, or 100%) identical to SEQ ID NO: 157, where the LCVD includes a histidine at one or more amino acid positions in SEQ ID NO: 157 selected from: 25, 32, 34, 36, 54, 56, 57, 58, 59, 60, 93, 96, 97, 99, 100, and 101.

In some examples, the first ABD includes an LCVD that is at least 90% (e.g., at least 92%, 94%, 96%, 98%, at least 99%, or 100%) identical to SEQ ID NO: 157, where the LCVD includes an alanine at position 38 in SEQ ID NO: 157.

In some examples the first ABD includes: an LCVD that is at least 90% (e.g., at least 92%, 94%, 96%, 98%, at least 99%, or 100%) identical to SEQ ID NO: 157, where the HCVD includes an alanine at position in SEQ ID NO 157, and an HCVD that is at least 9 d (e.g., at least 92%, 94% 96%, 98% at least, or 100%) identical to SEQ ID NO: 156, where the HCVD includes a histidine at one or more amino acid positions in SEQ ID NO: 156 selected from: 26, 29, 30, 33, 34, 51, 54, 56, 57, 59, 102, 103, and 106.

In some examples, the first ABD includes an LCVD that is at least 90 (e.g., at least 92%, 94%, 96%, 98%, at least 99%, or 100%) identical to SEQ ID NO: 157, where the LCVD includes an alanine at position 94 in SEQ ID NO: 157.

In some examples the first ABD includes: an LCVD that is at least 90%, (e.g., at least 92%, 94%, 96% 98%, at least 99%, or 100%) identical to SEQ ID NO: 157, where the LCVD includes an alanine at position 94 in SEQ ID NO 157, and an HCVD that is at least 90 (e.g., at least 92%, 94%, 896%, 98% at least 99%, or 100788) identical to SEQ ID NO: 156, where the HCVD includes a histidine at one or more amino acid positions in SEQ ID NO: 156 selected from: 26, 29, 30, 33, 34, 51, 54, 56, 57, 59, 102, 103, and 106.

TABLE 6
Exemplary Combinations of Amino Acid Positions in SEQ ID NO: 157 that can be Substituted

| | Col | | | | | | | | | | | | | | | | | | |
|---|---|---|---|---|---|---|---|---|---|---|---|---|---|---|---|---|---|---|---|
| | 1 | 2 | 3 | 4 | 5 | 6 | 7 | 8 | 9 | 10 | 11 | 12 | 13 | 14 | 15 | 16 | 17 | 18 | 19 |
| Pos | 25 | 32 | 34 | 36 | 54 | 56 | 57 | 58 | 59 | 60 | 93 | 94 | 96 | 97 | 99 | 100 | 101 | 38 | 94 |
| | | | | | | | | | | | | | | | | | | (A) | (A) |

Exemplary 2, 3, and 4 AA position combinations that can be substituted with histidine (or an alanine
at positions 38 and/or 94 of SEQ ID NO: 157)

| | |
|---|---|
| A | Each and every unique 2-position combination of X & Y, wherein X and Y are each selected |
| | (without replacement) from a SEQ ID NO: 157 AA position indicated in columns 1-19. |
| | Expressed another way, the combinations of positions recited in the following combinations of |
| | columns: 1 + 2, 1 + 3, 1 + 4, 1 + 5, 1 + 6, 1 + 7, 1 + 8, 1 + 9, 1 + 10, 1 + 11, 1 + 12, 1 + 13, 1 + 14, 1 + 15, 1 + 16, |
| | 1 + 17, 1 + 18, 1 + 19, 2 + 3, 2 + 4, 2 + 5, 2 + 6, 2 + 7, 2 + 8, 2 + 9, 2 + 10, 2 + 11, 2 + 12, 2 + 13, 2 + 14, 2 + 15, |
| | 2 + 16, 2 + 17, 2 + 18, 2 + 19, 3 + 4, 3 + 5, 3 + 6, 3 + 7, 3 + 8, 3 + 9, 3 + 10, 3 + 11, 3 + 12, 3 + 13, 3 + 14, 3 + 15, |
| | 3 + 16, 3 + 17, 3 + 18, 3 + 19, 4 + 5, 4 + 6, 4 + 7, 4 + 8, 4 + 9, 4 + 10, 4 + 11, 4 + 12, 4 + 13, 4 + 14, 4 + 15, 4 + 16, |
| | 4 + 17, 4 + 18, 4 + 19, 5 + 6, 5 + 7, 5 + 8, 5 + 9, 5 + 10, 5 + 11, 5 + 12, 5 + 13, 5 + 14, 5 + 15, 5 + 16, 5 + 17, |
| | 5 + 18, 5 + 19, 6 + 7, 6 + 8, 6 + 9, 6 + 10, 6 + 11, 6 + 12, 6 + 13, 6 + 14, 6 + 15, 6 + 16, 6 + 17, 6 + 18, 6 + 19, |
| | 7 + 8, 7 + 9, 7 + 10, 7 + 11, 7 + 12, 7 + 13, 7 + 14, 7 + 15, 7 + 16, 7 + 17, 7 + 18, 7 + 19, 8 + 9, 8 + 10, 8 + 11, |
| | 8 + 12, 8 + 13, 8 + 14, 8 + 15, 8 + 16, 8 + 17, 8 + 18, 8 + 19, 9 + 10, 9 + 11, 9 + 12, 9 + 13, 9 + 14, 9 + 15, 9 + 16, |
| | 9 + 17, 9 + 18, 9 + 19, 10 + 11, 10 + 12, 10 + 13, 10 + 14, 10 + 15, 10 + 16, 10 + 17, 10 + 18, 10 + 19, 11 + 12, |
| | 11 + 13, 11 + 14, 11 + 15, 11 + 16, 11 + 17, 11 + 18, 11 + 19, 12 + 13, 12 + 14, 12 + 15, 12 + 16, 12 + 17, |
| | 12 + 18, 12 + 19, 13 + 14, 13 + 15, 13 + 16, 13 + 17, 13 + 18, 13 + 19, 14 + 15, 14 + 16, 14 + 17, 14 + 18, |
| | 14 + 19, 15 + 16, 15 + 17, 15 + 18, 15 + 19, 16 + 17, 16 + 18, 16 + 19, 17 + 18, 17 + 19, and 18 + 19. |
| B | Each and every unique 3-position combination of X & Y & Z, wherein X, Y, and Z are each |
| | selected (without replacement) from a SEQ ID NO: 157 AA position indicated in columns 1-19. |
| | Expressed another way, the combinations of positions recited in the following combinations of |
| | columns: 1 + 2 + 3, 1 + 2 + 4, 1 + 2 + 5, 1 + 2 + 6, 1 + 2 + 7, 1 + 2 + 8, 1 + 2 + 9, 1 + 2 + 10, 1 + 2 + 11, 1 + 2 + 12, |
| | 1 + 2 + 13, 1 + 2 + 14, 1 + 2 + 15, 1 + 2 + 16, 1 + 2 + 17, 1 + 2 + 18, 1 + 2 + 19, 1 + 3 + 4, 1 + 3 + 5, 1 + 3 + 6, 1 + 3 + 7, |
| | 1 + 3 + 8, 1 + 3 + 9, 1 + 3 + 10, 1 + 3 + 11, 1 + 3 + 12, 1 + 3 + 13, 1 + 3 + 14, 1 + 3 + 15, 1 + 3 + 16, 1 + 3 + 17, |
| | 1 + 3 + 18, 1 + 3 + 19, 1 + 4 + 5, 1 + 4 + 6, 1 + 4 + 7, 1 + 4 + 8, 1 + 4 + 9, 1 + 4 + 10, 1 + 4 + 11, 1 + 4 + 12, 1 + 4 + 13, |
| | 1 + 4 + 14, 1 + 4 + 15, 1 + 4 + 16, 1 + 4 + 17, 1 + 4 + 18, 1 + 4 + 19, 1 + 5 + 6, 1 + 5 + 7, 1 + 5 + 8, 1 + 5 + 9, 1 + 5 + 10, |
| | 1 + 5 + 11, 1 + 5 + 12, 1 + 5 + 13, 1 + 5 + 14, 1 + 5 + 15, 1 + 5 + 16, 1 + 5 + 17, 1 + 5 + 18, 1 + 5 + 19, 1 + 6 + 7, |
| | 1 + 6 + 8, 1 + 6 + 9, 1 + 6 + 10, 1 + 6 + 11, 1 + 6 + 12, 1 + 6 + 13, 1 + 6 + 14, 1 + 6 + 15, 1 + 6 + 16, 1 + 6 + 17, |
| | 1 + 6 + 18, 1 + 6 + 19, 1 + 7 + 8, 1 + 7 + 9, 1 + 7 + 10, 1 + 7 + 11, 1 + 7 + 12, 1 + 7 + 13, 1 + 7 + 14, 1 + 7 + 15, |
| | 1 + 7 + 16, 1 + 7 + 17, 1 + 7 + 18, 1 + 7 + 19, 1 + 8 + 9, 1 + 8 + 10, 1 + 8 + 11, 1 + 8 + 12, 1 + 8 + 13, 1 + 8 + 14, |
| | 1 + 8 + 15, 1 + 8 + 16, 1 + 8 + 17, 1 + 8 + 18, 1 + 8 + 19, 1 + 9 + 10, 1 + 9 + 11, 1 + 9 + 12, 1 + 9 + 13, 1 + 9 + 14, |
| | 1 + 9 + 15, 1 + 9 + 16, 1 + 9 + 17, 1 + 9 + 18, 1 + 9 + 19, 1 + 10 + 11, 1 + 10 + 12, 1 + 10 + 13, 1 + 10 + 14, |
| | 1 + 10 + 15, 1 + 10 + 16, 1 + 10 + 17, 1 + 10 + 18, 1 + 10 + 19, 1 + 11 + 12, 1 + 11 + 13, 1 + 11 + 14, 1 + 11 + 15, |
| | 1 + 11 + 16, 1 + 11 + 17, 1 + 11 + 18, 1 + 11 + 19, 1 + 12 + 13, 1 + 12 + 14, 1 + 12 + 15, 1 + 12 + 16, 1 + 12 + 17, |
| | 1 + 12 + 18, 1 + 12 + 19, 1 + 13 + 14, 1 + 13 + 15, 1 + 13 + 16, 1 + 13 + 17, 1 + 13 + 18, 1 + 13 + 19, 1 + 14 + 15, |
| | 1 + 14 + 16, 1 + 14 + 17, 1 + 14 + 18, 1 + 14 + 19, 1 + 15 + 16, 1 + 15 + 17, 1 + 15 + 18, 1 + 15 + 19, 1 + 16 + 17, |
| | 1 + 16 + 18, 1 + 16 + 19, 1 + 17 + 18, 1 + 17 + 19, 1 + 18 + 19, 2 + 3 + 4, 2 + 3 + 5, 2 + 3 + 6, 2 + 3 + 7, 2 + 3 + 8, |
| | 2 + 3 + 9, 2 + 3 + 10, 2 + 3 + 11, 2 + 3 + 12, 2 + 3 + 13, 2 + 3 + 14, 2 + 3 + 15, 2 + 3 + 16, 2 + 3 + 17, 2 + 3 + 18, |
| | 2 + 3 + 19, 2 + 4 + 5, 2 + 4 + 6, 2 + 4 + 7, 2 + 4 + 8, 2 + 4 + 9, 2 + 4 + 10, 2 + 4 + 11, 2 + 4 + 12, 2 + 4 + 13, 2 + 4 + 14, |
| | 2 + 4 + 15, 2 + 4 + 16, 2 + 4 + 17, 2 + 4 + 18, 2 + 4 + 19, 2 + 5 + 6, 2 + 5 + 7, 2 + 5 + 8, 2 + 5 + 9, 2 + 5 + 10, 2 + 5 + 11, |
| | 2 + 5 + 12, 2 + 5 + 13, 2 + 5 + 14, 2 + 5 + 15, 2 + 5 + 16, 2 + 5 + 17, 2 + 5 + 18, 2 + 5 + 19, 2 + 6 + 7, 2 + 6 + 8, 2 + 6 + 9, |
| | 2 + 6 + 10, 2 + 6 + 11, 2 + 6 + 12, 2 + 6 + 13, 2 + 6 + 14, 2 + 6 + 15, 2 + 6 + 16, 2 + 6 + 17, 2 + 6 + 18, 2 + 6 + 19, |
| | 2 + 7 + 8, 2 + 7 + 9, 2 + 7 + 10, 2 + 7 + 11, 2 + 7 + 12, 2 + 7 + 13, 2 + 7 + 14, 2 + 7 + 15, 2 + 7 + 16, 2 + 7 + 17, |
| | 2 + 7 + 18, 2 + 7 + 19, 2 + 8 + 9, 2 + 8 + 10, 2 + 8 + 11, 2 + 8 + 12, 2 + 8 + 13, 2 + 8 + 14, 2 + 8 + 15, 2 + 8 + 16, |
| | 2 + 8 + 17, 2 + 8 + 18, 2 + 8 + 19, 2 + 9 + 10, 2 + 9 + 11, 2 + 9 + 12, 2 + 9 + 13, 2 + 9 + 14, 2 + 9 + 15, 2 + 9 + 16, |
| | 2 + 9 + 17, 2 + 9 + 18, 2 + 9 + 19, 2 + 10 + 11, 2 + 10 + 12, 2 + 10 + 13, 2 + 10 + 14, 2 + 10 + 15, 2 + 10 + 16, |
| | 2 + 10 + 17, 2 + 10 + 18, 2 + 10 + 19, 2 + 11 + 12, 2 + 11 + 13, 2 + 11 + 14, 2 + 11 + 15, 2 + 11 + 16, 2 + 11 + 17, |
| | 2 + 11 + 18, 2 + 11 + 19, 2 + 12 + 13, 2 + 12 + 14, 2 + 12 + 15, 2 + 12 + 16, 2 + 12 + 17, 2 + 12 + 18, 2 + 12 + 19, |
| | 2 + 13 + 14, 2 + 13 + 15, 2 + 13 + 16, 2 + 13 + 17, 2 + 13 + 18, 2 + 13 + 19, 2 + 14 + 15, 2 + 14 + 16, 2 + 14 + 17, |
| | 2 + 14 + 18, 2 + 14 + 19, 2 + 15 + 16, 2 + 15 + 17, 2 + 15 + 18, 2 + 15 + 19, 2 + 16 + 17, 2 + 16 + 18, 2 + 16 + 19, |
| | 2 + 17 + 18, 2 + 17 + 19, 2 + 18 + 19, 3 + 4 + 5, 3 + 4 + 6, 3 + 4 + 7, 3 + 4 + 8, 3 + 4 + 9, 3 + 4 + 10, 3 + 4 + 11, |
| | 3 + 4 + 12, 3 + 4 + 13, 3 + 4 + 14, 3 + 4 + 15, 3 + 4 + 16, 3 + 4 + 17, 3 + 4 + 18, 3 + 4 + 19, 3 + 5 + 6, 3 + 5 + 7, 3 + 5 + 8, |
| | 3 + 5 + 9, 3 + 5 + 10, 3 + 5 + 11, 3 + 5 + 12. 3 + 5 + 13, 3 + 5 + 14, 3 + 5 + 15, 3 + 5 + 16, 3 + 5 + 17, 3 + 5 + 18, |
| | 3 + 5 + 19, 3 + 6 + 7, 3 + 6 + 8, 3 + 6 + 9, 3 + 6 + 10, 3 + 6 + 11, 3 + 6 + 12, 3 + 6 + 13, 3 + 6 + 14, 3 + 6 + 15, 3 + 6 + 16, |
| | 3 + 6 + 17, 3 + 6 + 18, 3 + 6 + 19, 3 + 7 + 8, 3 + 7 + 9, 3 + 7 + 10, 3 + 7 + 11, 3 + 7 + 12, 3 + 7 + 13, 3 + 7 + 14, |
| | 3 + 7 + 15, 3 + 7 + 16, 3 + 7 + 17, 3 + 7 + 18, 3 + 7 + 19, 3 + 8 + 9, 3 + 8 + 10, 3 + 8 + 11, 3 + 8 + 12, 3 + 8 + 13, |
| | 3 + 8 + 14, 3 + 8 + 15, 3 + 8 + 16, 3 + 8 + 17, 3 + 8 + 18, 3 + 8 + 19, 3 + 9 + 10, 3 + 9 + 11, 3 + 9 + 12, 3 + 9 + 13, |
| | 3 + 9 + 14, 3 + 9 + 15, 3 + 9 + 16, 3 + 9 + 17, 3 + 9 + 18, 3 + 9 + 19, 3 + 10 + 11, 3 + 10 + 12, 3 + 10 + 13, 3 + 10 + 14, |
| | 3 + 10 + 15, 3 + 10 + 16, 3 + 10 + 17, 3 + 10 + 18, 3 + 10 + 19, 3 + 11 + 12, 3 + 11 + 13, 3 + 11 + 14, 3 + 11 + 15, |
| | 3 + 11 + 16, 3 + 11 + 17, 3 + 11 + 18, 3 + 11 + 19, 3 + 12 + 13, 3 + 12 + 14, 3 + 12 + 15, 3 + 12 + 16, 3 + 12 + 17, |
| | 3 + 12 + 18, 3 + 12 + 19, 3 + 13 + 14, 3 + 13 + 15, 3 + 13 + 16, 3 + 13 + 17, 3 + 13 + 18, 3 + 13 + 19, 3 + 14 + 15, |
| | 3 + 14 + 16, 3 + 14 + 17, 3 + 14 + 18, 3 + 14 + 19, 3 + 15 + 16, 3 + 15 + 17, 3 + 15 + 18, 3 + 15 + 19, 3 + 16 + 17, |
| | 3 + 16 + 18, 3 + 16 + 19, 3 + 17 + 18, 3 + 17 + 19, 3 + 18 + 19, 4 + 5 + 6, 4 + 5 + 7, 4 + 5 + 8, 4 + 5 + 9, 4 + 5 + 10, |
| | 4 + 5 + 11, 4 + 5 + 12, 4 + 5 + 13, 4 + 5 + 14, 4 + 5 + 15, 4 + 5 + 16, 4 + 5 + 17, 4 + 5 + 18, 4 + 5 + 19, 4 + 6 + 7, |
| | 4 + 6 + 8, 4 + 6 + 9, 4 + 6 + 10, 4 + 6 + 11, 4 + 6 + 12, 4 + 6 + 13, 4 + 6 + 14, 4 + 6 + 15, 4 + 6 + 16, 4 + 6 + 17, |
| | 4 + 6 + 18, 4 + 6 + 19, 4 + 7 + 8, 4 + 7 + 9, 4 + 7 + 10, 4 + 7 + 11, 4 + 7 + 12, 4 + 7 + 13, 4 + 7 + 14, 4 + 7 + 15, |
| | 4 + 7 + 16, 4 + 7 + 17, 4 + 7 + 18, 4 + 7 + 19, 4 + 8 + 9, 4 + 8 + 10, 4 + 8 + 11, 4 + 8 + 12, 4 + 8 + 13, 4 + 8 + 14, |
| | 4 + 8 + 15, 4 + 8 + 16, 4 + 8 + 17, 4 + 8 + 18, 4 + 8 + 19, 4 + 9 + 10, 4 + 9 + 11, 4 + 9 + 12, 4 + 9 + 13, 4 + 9 + 14, |
| | 4 + 9 + 15, 4 + 9 + 16, 4 + 9 + 17, 4 + 9 + 18, 4 + 9 + 19, 4 + 10 + 11, 4 + 10 + 12, 4 + 10 + 13, 4 + 10 + 14, |
| | 4 + 10 + 15, 4 + 10 + 16, 4 + 10 + 17, 4 + 10 + 18, 4 + 10 + 19, 4 + 11 + 12, 4 + 11 + 13, 4 + 11 + 14, 4 + 11 + 15, |
| | 4 + 11 + 16, 4 + 11 + 17, 4 + 11 + 18, 4 + 11 + 19, 4 + 12 + 13, 4 + 12 + 14, 4 + 12 + 15, 4 + 12 + 16, 4 + 12 + 17, |
| | 4 + 12 + 18, 4 + 12 + 19, 4 + 13 + 14, 4 + 13 + 15, 4 + 13 + 16, 4 + 13 + 17, 4 + 13 + 18, 4 + 13 + 19, 4 + 14 + 15, |
| | 4 + 14 + 16, 4 + 14 + 17, 4 + 14 + 18, 4 + 14 + 19, 4 + 15 + 16, 4 + 15 + 17, 4 + 15 + 18, 4 + 15 + 19, 4 + 16 + 17, |
| | 4 + 16 + 18, 4 + 16 + 19, 4 + 17 + 18, 4 + 17 + 19, 4 + 18 + 19, 5 + 6 + 7, 5 + 6 + 8, 5 + 6 + 9, 5 + 6 + 10, 5 + 6 + 11, |
| | 5 + 6 + 12, 5 + 6 + 13, 5 + 6 + 14, 5 + 6 + 15, 5 + 6 + 16, 5 + 6 + 17, 5 + 6 + 18, 5 + 6 + 19, 5 + 7 + 8, 5 + 7 + 9, |
| | 5 + 7 + 10, 5 + 7 + 11, 5 + 7 + 12, 5 + 7 + 13, 5 + 7 + 14, 5 + 7 + 15, 5 + 7 + 16, 5 + 7 + 17, 5 + 7 + 18, 5 + 7 + 19, |
| | 5 + 8 + 9, 5 + 8 + 10, 5 + 8 + 11, 5 + 8 + 12, 5 + 8 + 13, 5 + 8 + 14, 5 + 8 + 15, 5 + 8 + 16, 5 + 8 + 17, 5 + 8 + 18, |
| | 5 + 8 + 19, 5 + 9 + 10, 5 + 9 + 11, 5 + 9 + 12, 5 + 9 + 13, 5 + 9 + 14, 5 + 9 + 15, 5 + 9 + 16, 5 + 9 + 17, 5 + 9 + 18, |
| | 5 + 9 + 19, 5 + 10 + 11, 5 + 10 + 12, 5 + 10 + 13, 5 + 10 + 14, 5 + 10 + 15, 5 + 10 + 16, 5 + 10 + 17, 5 + 10 + 18, |
| | 5 + 10 + 19, 5 + 11 + 12, 5 + 11 + 13, 5 + 11 + 14, 5 + 11 + 15, 5 + 11 + 16, 5 + 11 + 17, 5 + 11 + 18, 5 + 11 + 19, |
| | 5 + 12 + 13, 5 + 12 + 14, 5 + 12 + 15, 5 + 12 + 16, 5 + 12 + 17, 5 + 12 + 18, 5 + 12 + 19, 5 + 13 + 14, 5 + 13 + 15, |
| | 5 + 13 + 16, 5 + 13 + 17, 5 + 13 + 18, 5 + 13 + 19, 5 + 14 + 15, 5 + 14 + 16, 5 + 14 + 17, 5 + 14 + 18, 5 + 14 + 19, |
| | 5 + 15 + 16, 5 + 15 + 17, 5 + 15 + 18, 5 + 15 + 19, 5 + 16 + 17, 5 + 16 + 18, 5 + 16 + 19, 5 + 17 + 18, 5 + 17 + 19, |
| | 5 + 18 + 19, 6 + 7 + 8, 6 + 7 + 9, 6 + 7 + 10, 6 + 7 + 11, 6 + 7 + 12, 6 + 7 + 13, 6 + 7 + 14, 6 + 7 + 15, 6 + 7 + 16, |
| | 6 + 7 + 17, 6 + 7 + 18, 6 + 7 + 19, 6 + 8 + 9, 6 + 8 + 10, 6 + 8 + 11, 6 + 8 + 12, 6 + 8 + 13, 6 + 8 + 14, 6 + 8 + 15, |
| | 6 + 8 + 16, 6 + 8 + 17, 6 + 8 + 18, 6 + 8 + 19, 6 + 9 + 10, 6 + 9 + 11, 6 + 9 + 12, 6 + 9 + 13, 6 + 9 + 14, 6 + 9 + 15, |
| | 6 + 9 + 16, 6 + 9 + 17, 6 + 9 + 18, 6 + 9 + 19, 6 + 10 + 11, 6 + 10 + 12, 6 + 10 + 13, 6 + 10 + 14, 6 + 10 + 15, |
| | 6 + 10 + 16, 6 + 10 + 17, 6 + 10 + 18, 6 + 10 + 19, 6 + 11 + 12, 6 + 11 + 13, 6 + 11 + 14, 6 + 11 + 15, 6 + 11 + 16, |
| | 6 + 11 + 17, 6 + 11 + 18, 6 + 11 + 19, 6 + 12 + 13, 6 + 12 + 14, 6 + 12 + 15, 6 + 12 + 16, 6 + 12 + 17, 6 + 12 + 18, |
| | 6 + 12 + 19, 6 + 13 + 14, 6 + 13 + 15, 6 + 13 + 16, 6 + 13 + 17, 6 + 13 + 18, 6 + 13 + 19, 6 + 14 + 15, 6 + 14 + 16, |
| | 6 + 14 + 17, 6 + 14 + 18, 6 + 14 + 19, 6 + 15 + 16, 6 + 15 + 17, 6 + 15 + 18, 6 + 15 + 19, 6 + 16 + 17, 6 + 16 + 18, |
| | 6 + 16 + 19, 6 + 17 + 18, 6 + 17 + 19, 6 + 18 + 19, 7 + 8 + 9, 7 + 8 + 10, 7 + 8 + 11, 7 + 8 + 12, 7 + 8 + 13, 7 + 8 + 14, |
| | 7 + 8 + 15, 7 + 8 + 16, 7 + 8 + 17, 7 + 8 + 18, 7 + 8 + 19, 7 + 9 + 10, 7 + 9 + 11, 7 + 9 + 12, 7 + 9 + 13, 7 + 9 + 14, |
| | 7 + 9 + 15, 7 + 9 + 16, 7 + 9 + 17, 7 + 9 + 18, 7 + 9 + 19, 7 + 10 + 11, 7 + 10 + 12, 7 + 10 + 13, 7 + 10 + 14, |
| | 7 + 10 + 15, 7 + 10 + 16, 7 + 10 + 17, 7 + 10 + 18, 7 + 10 + 19, 7 + 11 + 12, 7 + 11 + 13, 7 + 11 + 14, 7 + 11 + 15, |
| | 7 + 11 + 16, 7 + 11 + 17, 7 + 11 + 18, 7 + 11 + 19, 7 + 12 + 13, 7 + 12 + 14, 7 + 12 + 15, 7 + 12 + 16, 7 + 12 + 17, |
| | 7 + 12 + 18, 7 + 12 + 19, 7 + 13 + 14, 7 + 13 + 15, 7 + 13 + 16, 7 + 13 + 17, 7 + 13 + 18, 7 + 13 + 19, 7 + 14 + 15, |
| | 7 + 14 + 16, 7 + 14 + 17, 7 + 14 + 18, 7 + 14 + 19, 7 + 15 + 16, 7 + 15 + 17, 7 + 15 + 18, 7 + 15 + 19, 7 + 16 + 17, |
| | 7 + 16 + 18, 7 + 16 + 19, 7 + 17 + 18, 7 + 17 + 19, 7 + 18 + 19, 8 + 9 + 10, 8 + 9 + 11, 8 + 9 + 12, 8 + 9 + 13, |
| | 8 + 9 + 14, 8 + 9 + 15, 8 + 9 + 16, 8 + 9 + 17, 8 + 9 + 18, 8 + 9 + 19, 8 + 10 + 11, 8 + 10 + 12, 8 + 10 + 13, 8 + 10 + 14, |
| | 8 + 10 + 15, 8 + 10 + 16, 8 + 10 + 17, 8 + 10 + 18, 8 + 10 + 19, 8 + 11 + 12, 8 + 11 + 13, 8 + 11 + 14, 8 + 11 + 15, |
| | 8 + 11 + 16, 8 + 11 + 17, 8 + 11 + 18, 8 + 11 + 19, 8 + 12 + 13, 8 + 12 + 14, 8 + 12 + 15, 8 + 12 + 16, 8 + 12 + 17, |
| | 8 + 12 + 18, 8 + 12 + 19, 8 + 13 + 14, 8 + 13 + 15, 8 + 13 + 16, 8 + 13 + 17, 8 + 13 + 18, 8 + 13 + 19, 8 + 14 + 15, |
| | 8 + 14 + 16, 8 + 14 + 17, 8 + 14 + 18, 8 + 14 + 19, 8 + 15 + 16, 8 + 15 + 17, 8 + 15 + 18, 8 + 15 + 19, 8 + 16 + 17, |
| | 8 + 16 + 18, 8 + 16 + 19, 8 + 17 + 18, 8 + 17 + 19, 8 + 18 + 19, 9 + 10 + 11, 9 + 10 + 12, 9 + 10 + 13, 9 + 10 + 14, |
| | 9 + 10 + 15, 9 + 10 + 16, 9 + 10 + 17, 9 + 10 + 18, 9 + 10 + 19, 9 + 11 + 12, 9 + 11 + 13, 9 + 11 + 14, 9 + 11 + 15, |
| | 9 + 11 + 16, 9 + 11 + 17, 9 + 11 + 18, 9 + 11 + 19, 9 + 12 + 13, 9 + 12 + 14, 9 + 12 + 15, 9 + 12 + 16, 9 + 12 + 17, |
| | 9 + 12 + 18, 9 + 12 + 19, 9 + 13 + 14, 9 + 13 + 15, 9 + 13 + 16, 9 + 13 + 17, 9 + 13 + 18, 9 + 13 + 19, 9 + 14 + 15, |
| | 9 + 14 + 16, 9 + 14 + 17, 9 + 14 + 18, 9 + 14 + 19, 9 + 15 + 16, 9 + 15 + 17, 9 + 15 + 18, 9 + 15 + 19, 9 + 16 + 17, |
| | 9 + 16 + 18, 9 + 16 + 19, 9 + 17 + 18, 9 + 17 + 19, 9 + 18 + 19, 10 + 11 + 12, 10 + 11 + 13, 10 + 11 + 14, |
| | 10 + 11 + 15, 10 + 11 + 16, 10 + 11 + 17, 10 + 11 + 18, 10 + 11 + 19, 10 + 12 + 13, 10 + 12 + 14, 10 + 12 + 15, |
| | 10 + 12 + 16, 10 + 12 + 17, 10 + 12 + 18, 10 + 12 + 19, 10 + 13 + 14, 10 + 13 + 15, 10 + 13 + 16, 10 + 13 + 17, |
| | 10 + 13 + 18, 10 + 13 + 19, 10 + 14 + 15, 10 + 14 + 16, 10 + 14 + 17, 10 + 14 + 18, 10 + 14 + 19, 10 + 15 + 16, |
| | 10 + 15 + 17, 10 + 15 + 18, 10 + 15 + 19, 10 + 16 + 17, 10 + 16 + 18, 10 + 16 + 19, 10 + 17 + 18, 10 + 17 + 19, |
| | 10 + 18 + 19, 11 + 12 + 13, 11 + 12 + 14, 11 + 12 + 15, 11 + 12 + 16, 11 + 12 + 17, 11 + 12 + 18, 11 + 12 + 19, |
| | 11 + 13 + 14, 11 + 13 + 15, 11 + 13 + 16, 11 + 13 + 17, 11 + 13 + 18, 11 + 13 + 19, 11 + 14 + 15, 11 + 14 + 16, |
| | 11 + 14 + 17, 11 + 14 + 18, 11 + 14 + 19, 11 + 15 + 16, 11 + 15 + 17, 11 + 15 + 18, 11 + 15 + 19, 11 + 16 + 17, |
| | 11 + 16 + 18, 11 + 16 + 19, 11 + 17 + 18, 11 + 17 + 19, 11 + 18 + 19, 12 + 13 + 14, 12 + 13 + 15, 12 + 13 + 16, |
| | 12 + 13 + 17, 12 + 13 + 18, 12 + 13 + 19, 12 + 14 + 15, 12 + 14 + 16, 12 + 14 + 17, 12 + 14 + 18, 12 + 14 + 19, |
| | 12 + 15 + 16, 12 + 15 + 17, 12 + 15 + 18, 12 + 15 + 19, 12 + 16 + 17, 12 + 16 + 18, 12 + 16 + 19, 12 + 17 + 18, |
| | 12 + 17 + 19, 12 + 18 + 19, 13 + 14 + 15, 13 + 14 + 16, 13 + 14 + 17, 13 + 14 + 18, 13 + 14 + 19, 13 + 15 + 16, |
| | 13 + 15 + 17, 13 + 15 + 18, 13 + 15 + 19, 13 + 16 + 17, 13 + 16 + 18, 13 + 16 + 19, 13 + 17 + 18, 13 + 17 + 19, |
| | 13 + 18 + 19, 14 + 15 + 16, 14 + 15 + 17, 14 + 15 + 18, 14 + 15 + 19, 14 + 16 + 17, 14 + 16 + 18, 14 + 16 + 19, |
| | 14 + 17 + 18, 14 + 17 + 19, 14 + 18 + 19, 15 + 16 + 17, 15 + 16 + 18, 15 + 16 + 19, 15 + 17 + 18, 15 + 17 + 19, |
| | 15 + 18 + 19, 16 + 17 + 18, 16 + 17 + 19, 16 + 18 + 19, and 17 + 18 + 19. |
| C | Each and every unique 4-position combination of X & Y & Z & α, wherein X, Y, and Z are |
| | together one of the 3-position combinations recited in “B” (above), and α is selected from a |
| | SEQ ID NO: 157 amino acid position recited in one of columns 1 to 19, without replacement |
| | (i.e. X ≠ Y ≠ Z ≠ α). |

In some examples, the first ABD includes an HCVD that is at least 90%, 95%, or 98% identical to SEQ ID NO: 156, where the HCVD includes a histidine at any of the specific combinations of two or more amino acid positions in SEQ ID NO: 156 listed in Table 5; and an LCVD that that is at least 90%, 95%, or 98% identical to SEQ ID NO: 157, where the LCVD includes a histidine at any of the specific combinations of two or more amino acid positions in SEQ ID NO. 157 listed in Table 6.

In some examples, the first ABD includes an LCVD that is at least 90%, 95%, or 98% identical to SEQ ID NO: 157, where the LCVD includes a histidine at any of the specific combinations of two or more amino acid positions in SEQ ID NO: 157 listed in Table 6, and/or where the LCVD includes an alanine at position 38 or 94 in SEQ ID NO: 157; and an HCVD that that is at least 90%, 95%, or 98% identical to SEQ ID NO: 156, where the HCVD includes a histidine at any of the specific combinations of two or more amino acid positions in SEQ ID NO: 156 listed in Table 5.

In some examples, the first ABD comprises an LCVD comprising SEQ ID NO: 157, and an HCVD that is at least 90%, 95%, or 98% identical to SEQ ID NO: 156, where the HCVD includes a histidine at any of the specific combinations of two or more amino acid positions in SEQ ID NO: 156 listed in Table 5.

In some examples, the first ABD comprises an LCVD that is at least 90%, 95%, or 98% identical to SEQ ID NO: 157, where the LCVD includes a histidine at position 25 in SEQ ID NO: 157; and an HCVD that is at least 90%, 95%, or 98% identical to SEQ ID NO: 156, where the HCVD includes a histidine at any of the specific combinations of two or more amino acid positions in SEQ ID NO: 156 listed in Table 5.

In some examples, the first ABD comprises an LCVD that is at least 90%, 95%, or 98% identical to SEQ ID NO: 157, where the LCVD includes a histidine at position 32 in SEQ ID NO: 157, and an HCVD that is at least 90%, 95%, or 98% identical to SEQ ID NO: 156, where the HCVD includes a histidine at any of the specific combinations of two or more amino acid positions in SEQ ID NO: 156 listed in Table 5.

In some examples, the first ABD comprises an LCVD that is at least 90%, 95%, or 98% identical to SEQ ID NO: 157, where the LCVD includes a histidine at position 34 in SEQ ID NO: 157; and an HCVD that is at least 90%, 95%, or 98% identical to SEQ ID NO: 156, where the HCVD includes a histidine at any of the specific combinations of two or more amino acid positions in SEQ ID NO: 156 listed in Table 5.

In some examples, the first ABD comprises an LCVD that is at least 90%, 95%, or 98% identical to SEQ ID NO: 157, where the LCVD includes an alanine at position 38 in SEQ ID NO: 157; and an HCVD that is at least 90%, 95%, or 98% identical to SEQ ID NO: 156, wherein the HCVD includes a histidine at any of the specific combinations of two or more amino acid positions in SEQ ID NO: 156 listed in Table 5.

In some examples, the first ABD comprises an LCVD that is at least 90%, 95%, or 98% identical to SEQ ID NO: 157, where the LCVD includes an alanine at position 94 in SEQ ID NO: 157; and an HCVD that is at least 90%, 95%, or 98% identical to SEQ ID NO: 156, wherein the HCVD includes a histidine at any of the specific combinations of two or more amino acid positions in SEQ ID NO: 156 listed in Table 5.

In some examples, the first ABD comprises an LCVD that is at least 90%, 95%, or 98% identical to SEQ ID NO. 157, wherein the LCVD includes a histidine at position 36 in SEQ ID NO: 157; and an HCVD that is at least 90%, 95%, or 98% identical to SEQ ID NO: 156, wherein the HCVD includes a histidine at any of the specific combinations of two or more amino acid positions in SEQ ID NO: 156 listed in Table 5.

In some examples, the first ABD comprises an LCVD that is at least 90%, 95%, or 98% identical to SEQ ID NO: 157, wherein the LCVD includes a histidine at position 54 in SEQ ID NO: 157; and an HCVD that is at least 90%, 95%, or 98% identical to SEQ ID NO: 156, where the HCVD includes a histidine at any of the specific combinations of two or more amino acid positions in SEQ ID NO: 156 listed in Table 5.

In some examples, the first ABD comprises an LCVD that is at least 90%, 95%, or 98% identical to SEQ ID NO: 157, wherein the LCVD includes a histidine at position 56 in SEQ ID NO: 157; and an HCVD that is at least 90%, 95%, or 98% identical to SEQ ID NO: 156, where the HCVD includes a histidine at any of the specific combinations of two or more amino acid positions in SEQ ID NO: 156 listed in Table 5.

In some examples, the first ABD comprises an LCVD that is at least 90%, 95%, or 98% identical to SEQ ID NO: 157, wherein the LCVD includes a histidine at position 57 in SEQ ID NO: 157; and an HCVD that is at least 90%, 95%, or 98% identical to SEQ ID NO: 156, where the HCVD includes a histidine at any of the specific combinations of two or more amino acid positions in SEQ ID NO: 156 listed in Table 5.

In some examples, the first ABD comprises an LCVD that is at least 90%, 95%, or 98% identical to SEQ ID NO: 157, wherein the LCVD includes a histidine at position 58 in SEQ ID NO: 157; and an HCVD that is at least 90%, 95%, or 98% identical to SEQ ID NO: 156, where the HCVD includes a histidine at any of the specific combinations of two or more amino acid positions in SEQ ID NO: 156 listed in Table 5.

In some examples, the first ABD comprises an LCVD that is at least 90%, 95%, or 98% identical to SEQ ID NO. 157, wherein the LCVD includes a histidine at position 59 in SEQ ID NO: 157; and an HCVD that is at least 90%, 95%, or 98% identical to SEQ ID NO: 156, where the HCVD includes a histidine at any of the specific combinations of two or more amino acid positions in SEQ ID NO: 156 listed in Table 5.

In some examples, the first ABD comprises an LCVD that is at least 90%, 95%, or 98% identical to SEQ ID NO. 157, wherein the LCVD includes a histidine at position 60 in SEQ ID NO: 157; and an HCVD that is at least 90° %, 95%, or 98% identical to SEQ ID NO: 156, where the HCVD includes a histidine at any of the specific combinations of two or more amino acid positions in SEQ ID NO: 156 listed in Table 5.

In some examples, the first ABD comprises an LCVD that is at least 90%, 95%, or 98% identical to SEQ ID NO: 157, wherein the LCVD includes a histidine at position 93 in SEQ ID NO: 157; and an HCVD that is at least 90° %, 95%, or 98% identical to SEQ ID NO: 156, where the HCVD includes a histidine at any of the specific combinations of two or more amino acid positions in SEQ ID NO: 156 listed in Table 5.

In some examples, the first ABD comprises an LCVD that is at least 90%, 95%, or 98% identical to SEQ ID NO: 157, wherein the LCVD includes a histidine at position 94 in SEQ ID NO: 157; and an HCVD that is at least 90%, 95%, or 98% identical to SEQ ID NO: 156, where the HCVD includes a histidine at any of the specific combinations of two or more amino acid positions in SEQ ID NO: 156 listed in Table 5.

In some examples, the first ABD comprises an LCVD that is at least 90%, 95%, or 98% identical to SEQ ID NO: 157, wherein the LCVD includes a histidine at position 96 in SEQ ID NO: 157; and an HCVD that is at least 90%, 95%, or 98% identical to SEQ ID NO: 156, where the HCVD includes a histidine at any of the specific combinations of two or more amino acid positions in SEQ ID NO: 156 listed in Table 5.

In some examples, the first ABD comprises an LCVD that is at least 90%, 95%, or 98% identical to SEQ ID NO: 157, wherein the LCVD includes a histidine at position 97 in SEQ ID NO: 157; and an HCVD that is at least 90%, 95%, or 98% identical to SEQ ID NO: 156, where the HCVD includes a histidine at any of the specific combinations of two or more amino acid positions in SEQ ID NO: 156 listed in Table 5.

In some examples, the first ABD comprises an LCVD that is at least 90%, 95%, or 98% identical to SEQ ID NO. 157, wherein the LCVD includes a histidine at position 99 in SEQ ID NO: 157; and an HCVD that is at least 90%, 95%, or 98% identical to SEQ ID NO: 156, where the HCVD includes a histidine at any of the specific combinations of two or more amino acid positions in SEQ ID NO: 156 listed in Table 5.

In some examples, the first ABD comprises an LCVD that is at least 90%, 95%, or 98% identical to SEQ ID NO: 157, wherein the LCVD includes a histidine at position 100 in SEQ ID NO: 157; and an HCVD that is at least 90° %, 95%, or 98% identical to SEQ ID NO: 156, where the HCVD includes a histidine at any of the specific combinations of two or more amino acid positions in SEQ ID NO: 156 listed in Table 5.

In some examples, the first ABD comprises an LCVD that is at least 90%, 95%, or 98% identical to SEQ ID NO: 157, wherein the LCVD includes a histidine at position 101 in SEQ ID NO: 157; and an HCVD that is at least 90° %, 95%, or 98% identical to SEQ ID NO: 156, where the HCVD includes a histidine at any of the specific combinations of two or more amino acid positions in SEQ ID NO: 156 listed in Table 5.

In some examples, the first ABD comprises an HCVD that is at least 90%, 95%, or 98% identical to SEQ ID NO: 156, wherein the HCVD includes a histidine at any one of positions 26, 29, 30, 33, 34, 51, 54, 56, 57, 59, 102, 103, and 106 in SEQ ID NO: 156; and an LCVD that is at least 90%, 95%, or 98% identical to SEQ ID NO: 157, where the LCVD includes a histidine at any of the specific combinations of two or more amino acid positions in SEQ ID NO: 157 listed in Table 6.

In some examples, the first ABD comprises an HCVD of SEQ ID NO: 156, one of the sequences set forth in SEQ ID NOs: 164-202, or one of the sequences set forth in SEQ ID NOs: 427-457, and/or the first ABD includes an LCVD of SEQ ID NO: 157, one of the sequences set forth in SEQ ID NOs: 203-233, or one of the sequences set forth in SEQ ID NOs: 458-492.

In some examples of the ABPCs, the first ABD includes an LCVD comprising the sequence of SEQ ID NO: 157, one of the sequences set forth in SEQ ID NOs: 203-233, or one of the sequences set forth in SEQ ID NOs: 458-492, and an HCVD comprising the sequence of SEQ ID NO: 156, one of the sequences set forth in SEQ ID NOs: 164-202, or one of the sequences set forth in 427-457.

In some examples, the first ABD includes an HCVD comprising the sequence of SEQ ID NO: 156, and an LCVD comprising the sequence of SEQ ID NO: 157, one of the sequences set forth in SEQ ID NOs: 203-233, or one of the sequences set forth in SEQ ID NOs: 458-492.

In some examples, the first ABD includes an HCVD comprising the sequence of SEQ ID NO: 164, and an LCVD comprising the sequence of SEQ ID NO: 157, one of the sequences set forth in SEQ ID NOs: 203-233, or one of the sequences set forth in SEQ ID NOs: 458-492.

In some examples, the first ABD includes an HCVD comprising the sequence of SEQ ID NO: 165, and an LCVD comprising the sequence of SEQ ID NO: 157, one of the sequences set forth in SEQ ID NOs: 203-233, or one of the sequences set forth in SEQ ID NOs: 458-492.

In some examples, the first ABD includes an HCVD comprising the sequence of SEQ ID NO: 166, and an LCVD comprising the sequence of SEQ ID NO: 157, one of the sequences set forth in SEQ ID NOs: 203-233, or one of the sequences set forth in SEQ ID NOs: 458-492.

In some examples, the first ABD includes an HCVD comprising the sequence of SEQ ID NO: 167, and an LCVD comprising the sequence of SEQ ID NO: 157, one of the sequences set forth in SEQ ID NOs: 203-233, or one of the sequences set forth in SEQ ID NOs: 458-492.

In some examples, the first ABD includes an HCVD comprising the sequence of SEQ ID NO: 168, and an LCVD comprising the sequence of SEQ ID NO: 157, one of the sequences set forth in SEQ ID NOs: 203-233, or one of the sequences set forth in SEQ ID NOs: 458-492.

In some examples, the first ABD includes an HCVD comprising the sequence of SEQ ID NO: 169, and an LCVD comprising the sequence of SEQ ID NO: 157, one of the sequences set forth in SEQ ID NOs: 203-233, or one of the sequences set forth in SEQ ID NOs: 458-492.

In some examples, the first ABD includes an HCVD comprising the sequence of SEQ ID NO: 170, and an LCVD comprising the sequence of SEQ ID NO: 157, one of the sequences set forth in SEQ ID NOs: 203-233, or one of the sequences set forth in SEQ ID NOs: 458-492.

In some examples, the first ABD includes an HCVD comprising the sequence of SEQ ID NO: 171, and an LCVD comprising the sequence of SEQ ID NO: 157, one of the sequences set forth in SEQ ID NOs: 203-233, or one of the sequences set forth in SEQ ID NOs: 458-492.

In some examples, the first ABD includes an HCVD comprising the sequence of SEQ ID NO: 172, and an LCVD comprising the sequence of SEQ ID NO: 157, one of the sequences set forth in SEQ ID NOs: 203-233, or one of the sequences set forth in SEQ ID NOs: 458-492.

In some examples, the first ABD includes an HCVD comprising the sequence of SEQ ID NO: 173, and an LCVD comprising the sequence of SEQ ID NO: 157, one of the sequences set forth in SEQ ID NOs: 203-233, or one of the sequences set forth in SEQ ID NOs: 458-492.

In some examples, the first ABD includes an HCVD comprising the sequence of SEQ ID NO: 174, and an LCVD comprising the sequence of SEQ ID NO: 157, one of the sequences set forth in SEQ ID NOs: 203-233, or one of the sequences set forth in SEQ ID NOs: 458-492.

In some examples, the first ABD includes an HCVD comprising the sequence of SEQ ID NO: 175, and an LCVD comprising the sequence of SEQ ID NO: 157, one of the sequences set forth in SEQ ID NOs: 203-233, or one of the sequences set forth in SEQ ID NOs: 458-492.

In some examples, the first ABD includes an HCVD comprising the sequence of SEQ ID NO: 176, and an LCVD comprising the sequence of SEQ ID NO: 157, one of the sequences set forth in SEQ ID NOs: 203-233, or one of the sequences set forth in SEQ ID NOs: 458-492.

In some examples, the first ABD includes an HCVD comprising the sequence of SEQ ID NO: 177, and an LCVD comprising the sequence of SEQ ID NO: 157, one of the sequences set forth in SEQ ID NOs: 203-233, or one of the sequences set forth in SEQ ID NOs: 458-492.

In some examples, the first ABD includes an HCVD comprising the sequence of SEQ ID NO: 178, and an LCVD comprising the sequence of SEQ ID NO: 157, one of the sequences set forth in SEQ ID NOs: 203-233, or one of the sequences set forth in SEQ ID NOs: 458-492.

In some examples, the first ABD includes an HCVD comprising the sequence of SEQ ID NO: 179, and an LCVD comprising the sequence of SEQ ID NO: 157, one of the sequences set forth in SEQ ID NOs: 203-233, or one of the sequences set forth in SEQ ID NOs: 458-492.

In some examples, the first ABD includes an HCVD comprising the sequence of SEQ ID NO: 180, and an LCVD comprising the sequence of SEQ ID NO: 157, one of the sequences set forth in SEQ ID NOs: 203-233, or one of the sequences set forth in SEQ ID NOs: 458-492.

In some examples, the first ABD includes an HCVD comprising the sequence of SEQ ID NO: 181, and an LCVD comprising the sequence of SEQ ID NO: 157, one of the sequences set forth in SEQ ID NOs: 203-233, or one of the sequences set forth in SEQ ID NOs: 458-492.

In some examples, the first ABD includes an HCVD comprising the sequence of SEQ ID NO: 182, and an LCVD comprising the sequence of SEQ ID NO: 157, one of the sequences set forth in SEQ ID NOs: 203-233, or one of the sequences set forth in SEQ ID NOs: 458-492.

In some examples, the first ABD includes an HCVD comprising the sequence of SEQ ID NO: 183, and an LCVD comprising the sequence of SEQ ID NO: 157, one of the sequences set forth in SEQ ID NOs: 203-233, or one of the sequences set forth in SEQ ID NOs: 458-492.

In some examples, the first ABD includes an HCVD comprising the sequence of SEQ ID NO: 184, and an LCVD comprising the sequence of SEQ ID NO: 157, one of the sequences set forth in SEQ ID NOs: 203-233, or one of the sequences set forth in SEQ ID NOs: 458-492.

In some examples, the first ABD includes an HCVD comprising the sequence of SEQ ID NO: 185, and an LCVD comprising the sequence of SEQ ID NO: 157, one of the sequences set forth in SEQ ID NOs: 203-233, or one of the sequences set forth in SEQ ID NOs: 458-492.

In some examples, the first ABD includes an HCVD comprising the sequence of SEQ ID NO: 186, and an LCVD comprising the sequence of SEQ ID NO: 157, one of the sequences set forth in SEQ ID NOs: 203-233, or one of the sequences set forth in SEQ ID NOs: 458-492.

In some examples, the first ABD includes an HCVD comprising the sequence of SEQ ID NO: 187, and an LCVD comprising the sequence of SEQ ID NO: 157, one of the sequences set forth in SEQ ID NOs: 203-233, or one of the sequences set forth in SEQ ID NOs: 458-492.

In some examples, the first ABD includes an HCVD comprising the sequence of SEQ ID NO: 188, and an LCVD comprising the sequence of SEQ ID NO: 157, one of the sequences set forth in SEQ ID NOs: 203-233, or one of the sequences set forth in SEQ ID NOs: 458-492.

In some examples, the first ABD includes an HCVD comprising the sequence of SEQ ID NO: 189, and an LCVD comprising the sequence of SEQ ID NO: 157, one of the sequences set forth in SEQ ID NOs: 203-233, or one of the sequences set forth in SEQ ID NOs: 458-492.

In some examples, the first ABD includes an HCVD comprising the sequence of SEQ ID NO: 190, and an LCVD comprising the sequence of SEQ ID NO: 157, one of the sequences set forth in SEQ ID NOs: 203-233, or one of the sequences set forth in SEQ ID NOs: 458-492.

In some examples, the first ABD includes an HCVD comprising the sequence of SEQ ID NO: 191, and an LCVD comprising the sequence of SEQ ID NO: 157, one of the sequences set forth in SEQ ID NOs: 203-233, or one of the sequences set forth in SEQ ID NOs: 458-492.

In some examples, the first ABD includes an HCVD comprising the sequence of SEQ ID NO: 192, and an LCVD comprising the sequence of SEQ ID NO: 157, one of the sequences set forth in SEQ ID NOs: 203-233, or one of the sequences set forth in SEQ ID NOs: 458-492.

In some examples, the first ABD includes an HCVD comprising the sequence of SEQ ID NO: 193, and an LCVD comprising the sequence of SEQ ID NO: 157, one of the sequences set forth in SEQ ID NOs: 203-233, or one of the sequences set forth in SEQ ID NOs: 458-492.

In some examples, the first ABD includes an HCVD comprising the sequence of SEQ ID NO: 194, and an LCVD comprising the sequence of SEQ ID NO: 157, one of the sequences set forth in SEQ ID NOs: 203-233, or one of the sequences set forth in SEQ ID NOs: 458-492.

In some examples, the first ABD includes an HCVD comprising the sequence of SEQ ID NO: 195, and an LCVD comprising the sequence of SEQ ID NO: 157, one of the sequences set forth in SEQ ID NOs: 203-233, or one of the sequences set forth in SEQ ID NOs: 458-492.

In some examples, the first ABD includes an HCVD comprising the sequence of SEQ ID NO: 196, and an LCVD comprising the sequence of SEQ ID NO: 157, one of the sequences set forth in SEQ ID NOs: 203-233, or one of the sequences set forth in SEQ ID NOs: 458-492.

In some examples, the first ABD includes an HCVD comprising the sequence of SEQ ID NO: 197, and an LCVD comprising the sequence of SEQ ID NO: 157, one of the sequences set forth in SEQ ID NOs: 203-233, or one of the sequences set forth in SEQ ID NOs: 458-492.

In some examples, the first ABD includes an HCVD comprising the sequence of SEQ ID NO: 198, and an LCVD comprising the sequence of SEQ ID NO: 157, one of the sequences set forth in SEQ ID NOs: 203-233, or one of the sequences set forth in SEQ ID NOs: 458-492.

In some examples, the first ABD includes an HCVD comprising the sequence of SEQ ID NO: 199, and an LCVD comprising the sequence of SEQ ID NO: 157, one of the sequences set forth in SEQ ID NOs: 203-233, or one of the sequences set forth in SEQ ID NOs: 458-492.

In some examples, the first ABD includes an HCVD comprising the sequence of SEQ ID NO: 200, and an LCVD comprising the sequence of SEQ ID NO: 157, one of the sequences set forth in SEQ ID NOs: 203-233, or one of the sequences set forth in SEQ ID NOs: 458-492.

In some examples, the first ABD includes an HCVD comprising the sequence of SEQ ID NO: 201, and an LCVD comprising the sequence of SEQ ID NO: 157, one of the sequences set forth in SEQ ID NOs: 203-233, or one of the sequences set forth in SEQ ID NOs: 458-492.

In some examples, the first ABD includes an HCVD comprising the sequence of SEQ ID NO: 202, and an LCVD comprising the sequence of SEQ ID NO: 157, one of the sequences set forth in SEQ ID NOs: 203-233, or one of the sequences set forth in SEQ ID NOs: 458-492.

In some examples, the first ABD includes an HCVD comprising the sequence of SEQ ID NO: 427, and an LCVD comprising the sequence of SEQ ID NO: 157, one of the sequences set forth in SEQ ID NOs: 203-233, or one of the sequences set forth in SEQ ID NOs: 458-492.

In some examples, the first ABD includes an HCVD comprising the sequence of SEQ ID NO: 428, and an LCVD comprising the sequence of SEQ ID NO: 157, one of the sequences set forth in SEQ ID NOs: 203-233, or one of the sequences set forth in SEQ ID NOs: 458-492.

In some examples, the first ABD includes an HCVD comprising the sequence of SEQ ID NO: 429, and an LCVD comprising the sequence of SEQ ID NO: 157, one of the sequences set forth in SEQ ID NOs: 203-233, or one of the sequences set forth in SEQ ID NOs: 458-492.

In some examples, the first ABD includes an HCVD comprising the sequence of SEQ ID NO: 430, and an LCVD comprising the sequence of SEQ ID NO: 157, one of the sequences set forth in SEQ ID NOs: 203-233, or one of the sequences set forth in SEQ ID NOs: 458-492.

In some examples, the first ABD includes an HCVD comprising the sequence of SEQ ID NO: 431, and an LCVD comprising the sequence of SEQ ID NO: 157, one of the sequences set forth in SEQ ID NOs: 203-233, or one of the sequences set forth in SEQ ID NOs: 458-492.

In some examples, the first ABD includes an HCVD comprising the sequence of SEQ ID NO: 432, and an LCVD comprising the sequence of SEQ ID NO: 157, one of the sequences set forth in SEQ ID NOs: 203-233, or one of the sequences set forth in SEQ ID NOs: 458-492.

In some examples, the first ABD includes an HCVD comprising the sequence of SEQ ID NO: 433, and an LCVD comprising the sequence of SEQ ID NO: 157, one of the sequences set forth in SEQ ID NOs: 203-233, or one of the sequences set forth in SEQ ID NOs: 458-492.

In some examples, the first ABD includes an HCVD comprising the sequence of SEQ ID NO: 434, and an LCVD comprising the sequence of SEQ ID NO: 157, one of the sequences set forth in SEQ ID NOs: 203-233, or one of the sequences set forth in SEQ ID NOs: 458-492.

In some examples, the first ABD includes an HCVD comprising the sequence of SEQ ID NO: 435, and an LCVD comprising the sequence of SEQ ID NO: 157, one of the sequences set forth in SEQ ID NOs: 203-233, or one of the sequences set forth in SEQ ID NOs: 458-492.

In some examples, the first ABD includes an HCVD comprising the sequence of SEQ ID NO: 436, and an LCVD comprising the sequence of SEQ ID NO: 157, one of the sequences set forth in SEQ ID NOs: 203-233, or one of the sequences set forth in SEQ ID NOs: 458-492.

In some examples, the first ABD includes an HCVD comprising the sequence of SEQ ID NO: 437, and an LCVD comprising the sequence of SEQ ID NO: 157, one of the sequences set forth in SEQ ID NOs: 203-233, or one of the sequences set forth in SEQ ID NOs: 458-492.

In some examples, the first ABD includes an HCVD comprising the sequence of SEQ ID NO: 438, and an LCVD comprising the sequence of SEQ ID NO: 157, one of the sequences set forth in SEQ ID NOs: 203-233, or one of the sequences set forth in SEQ ID NOs: 458-492.

In some examples, the first ABD includes an HCVD comprising the sequence of SEQ ID NO: 439, and an LCVD comprising the sequence of SEQ ID NO: 157, one of the sequences set forth in SEQ ID NOs: 203-233, or one of the sequences set forth in SEQ ID NOs: 458-492.

In some examples, the first ABD includes an HCVD comprising the sequence of SEQ ID NO: 440, and an LCVD comprising the sequence of SEQ ID NO: 157, one of the sequences set forth in SEQ ID NOs: 203-233, or one of the sequences set forth in SEQ ID NOs: 458-492.

In some examples, the first ABD includes an HCVD comprising the sequence of SEQ ID NO: 441, and an LCVD comprising the sequence of SEQ ID NO: 157, one of the sequences set forth in SEQ ID NOs: 203-233, or one of the sequences set forth in SEQ ID NOs: 458-492.

In some examples, the first ABD includes an HCVD comprising the sequence of SEQ ID NO: 442, and an LCVD comprising the sequence of SEQ ID NO: 157, one of the sequences set forth in SEQ ID NOs: 203-233, or one of the sequences set forth in SEQ ID NOs: 458-492.

In some examples, the first ABD includes an HCVD comprising the sequence of SEQ ID NO: 443, and an LCVD comprising the sequence of SEQ ID NO: 157, one of the sequences set forth in SEQ ID NOs: 203-233, or one of the sequences set forth in SEQ ID NOs: 458-492.

In some examples, the first ABD includes an HCVD comprising the sequence of SEQ ID NO: 444, and an LCVD comprising the sequence of SEQ ID NO: 157, one of the sequences set forth in SEQ ID NOs: 203-233, or one of the sequences set forth in SEQ ID NOs: 458-492.

In some examples, the first ABD includes an HCVD comprising the sequence of SEQ ID NO: 445, and an LCVD comprising the sequence of SEQ ID NO: 157, one of the sequences set forth in SEQ ID NOs: 203-233, or one of the sequences set forth in SEQ ID NOs: 458-492.

In some examples, the first ABD includes an HCVD comprising the sequence of SEQ ID NO: 446, and an LCVD comprising the sequence of SEQ ID NO: 157, one of the sequences set forth in SEQ ID NOs: 203-233, or one of the sequences set forth in SEQ ID NOs: 458-492.

In some examples, the first ABD includes an HCVD comprising the sequence of SEQ ID NO: 447, and an LCVD comprising the sequence of SEQ ID NO: 157, one of the sequences set forth in SEQ ID NOs: 203-233, or one of the sequences set forth in SEQ ID NOs: 458-492.

In some examples, the first ABD includes an HCVD comprising the sequence of SEQ ID NO: 448, and an LCVD comprising the sequence of SEQ ID NO: 157, one of the sequences set forth in SEQ ID NOs: 203-233, or one of the sequences set forth in SEQ ID NOs: 458-492.

In some examples, the first ABD includes an HCVD comprising the sequence of SEQ ID NO: 449, and an LCVD comprising the sequence of SEQ ID NO: 157, one of the sequences set forth in SEQ ID NOs: 203-233, or one of the sequences set forth in SEQ ID NOs: 458-492.

In some examples, the first ABD includes an HCVD comprising the sequence of SEQ ID NO: 450, and an LCVD comprising the sequence of SEQ ID NO: 157, one of the sequences set forth in SEQ ID NOs: 203-233, or one of the sequences set forth in SEQ ID NOs: 458-492.

In some examples, the first ABD includes an HCVD comprising the sequence of SEQ ID NO: 451, and an LCVD comprising the sequence of SEQ ID NO: 157, one of the sequences set forth in SEQ ID NOs: 203-233, or one of the sequences set forth in SEQ ID NOs: 458-492.

In some examples, the first ABD includes an HCVD comprising the sequence of SEQ ID NO: 452, and an LCVD comprising the sequence of SEQ ID NO: 157, one of the sequences set forth in SEQ ID NOs: 203-233, or one of the sequences set forth in SEQ ID NOs: 458-492.

In some examples, the first ABD includes an HCVD comprising the sequence of SEQ ID NO: 453, and an LCVD comprising the sequence of SEQ ID NO: 157, one of the sequences set forth in SEQ ID NOs: 203-233, or one of the sequences set forth in SEQ ID NOs: 458-492.

In some examples, the first ABD includes an HCVD comprising the sequence of SEQ ID NO: 454, and an LCVD comprising the sequence of SEQ ID NO: 157, one of the sequences set forth in SEQ ID NOs: 203-233, or one of the sequences set forth in SEQ ID NOs: 458-492.

In some examples, the first ABD includes an HCVD comprising the sequence of SEQ ID NO: 455, and an LCVD comprising the sequence of SEQ ID NO: 157, one of the sequences set forth in SEQ ID NOs: 203-233, or one of the sequences set forth in SEQ ID NOs: 458-492.

In some examples, the first ABD includes an HCVD comprising the sequence of SEQ ID NO: 456, and an LCVD comprising the sequence of SEQ ID NO: 157, one of the sequences set forth in SEQ ID NOs: 203-233, or one of the sequences set forth in SEQ ID NOs: 458-492.

In some examples, the first ABD includes an HCVD comprising the sequence of SEQ ID NO: 457, and an LCVD comprising the sequence of SEQ ID NO: 157, one of the sequences set forth in SEQ ID NOs: 203-233, or one of the sequences set forth in SEQ ID NOs: 458-492.

In some examples, the first ABD includes an LCVD comprising the sequence of SEQ ID NO: 157, and an HCVD comprising the sequence of SEQ ID NO: 156, one of the sequences set forth in SEQ ID NOs: 164-202, or one of the sequences set forth in 427-457.

In some examples, the first ABD includes an LCVD comprising the sequence of SEQ ID NO: 203, and an HCVD comprising the sequence of SEQ ID NO: 156, one of the sequences set forth in SEQ ID NOs: 164-202, or one of the sequences set forth in 427-457.

In some examples, the first ABD includes an LCVD comprising the sequence of SEQ ID NO: 204, and an HCVD comprising the sequence of SEQ ID NO: 156, one of the sequences set forth in SEQ ID NOs: 164-202, or one of the sequences set forth in 427-457.

In some examples, the first ABD includes an LCVD comprising the sequence of SEQ ID NO: 205, and an HCVD comprising the sequence of SEQ ID NO: 156, one of the sequences set forth in SEQ ID NOs: 164-202, or one of the sequences set forth in 427-457.

In some examples, the first ABD includes an LCVD comprising the sequence of SEQ ID NO: 206, and an HCVD comprising the sequence of SEQ ID NO: 156, one of the sequences set forth in SEQ ID NOs: 164-202, or one of the sequences set forth in 427-457.

In some examples, the first ABD includes an LCVD comprising the sequence of SEQ ID NO: 207, and an HCVD comprising the sequence of SEQ ID NO: 156, one of the sequences set forth in SEQ ID NOs: 164-202, or one of the sequences set forth in 427457.

In some examples, the first ABD includes an LCVD comprising the sequence of SEQ ID NO: 208, and an HCVD comprising the sequence of SEQ ID NO: 156, one of the sequences set forth in SEQ ID NOs: 164-202, or one of the sequences set forth in 427-457.

In some examples, the first ABD includes an LCVD comprising the sequence of SEQ ID NO: 209, and an HCVD comprising the sequence of SEQ ID NO: 156, one of the sequences set forth in SEQ ID NOs: 164-202, or one of the sequences set forth in 427457.

In some examples, the first ABD includes an LCVD comprising the sequence of SEQ ID NO: 210, and an HCVD comprising the sequence of SEQ ID NO: 156, one of the sequences set forth in SEQ ID NOs: 164-202, or one of the sequences set forth in 427457.

In some examples, the first ABD includes an LCVD comprising the sequence of SEQ ID NO: 211, and an HCVD comprising the sequence of SEQ ID NO: 156, one of the sequences set forth in SEQ ID NOs: 164-202, or one of the sequences set forth in 427457.

In some examples, the first ABD includes an LCVD comprising the sequence of SEQ ID NO: 212, and an HCVD comprising the sequence of SEQ ID NO: 156, one of the sequences set forth in SEQ ID NOs: 164-202, or one of the sequences set forth in 427-457.

In some examples, the first ABD includes an LCVD comprising the sequence of SEQ ID NO: 213, and an HCVD comprising the sequence of SEQ ID NO: 156, one of the sequences set forth in SEQ ID NOs: 164-202, or one of the sequences set forth in 427457.

In some examples, the first ABD includes an LCVD comprising the sequence of SEQ ID NO: 214, and an HCVD comprising the sequence of SEQ ID NO: 156, one of the sequences set forth in SEQ ID NOs: 164-202, or one of the sequences set forth in 427457.

In some examples, the first ABD includes an LCVD comprising the sequence of SEQ ID NO: 215, and an HCVD comprising the sequence of SEQ ID NO: 156, one of the sequences set forth in SEQ ID NOs: 164-202, or one of the sequences set forth in 427457.

In some examples, the first ABD includes an LCVD comprising the sequence of SEQ ID NO: 216, and an HCVD comprising the sequence of SEQ ID NO: 156, one of the sequences set forth in SEQ ID NOs: 164-202, or one of the sequences set forth in 427457.

In some examples, the first ABD includes an LCVD comprising the sequence of SEQ ID NO: 217, and an HCVD comprising the sequence of SEQ ID NO: 156, one of the sequences set forth in SEQ ID NOs: 164-202, or one of the sequences set forth in 427457.

In some examples, the first ABD includes an LCVD comprising the sequence of SEQ ID NO: 218, and an HCVD comprising the sequence of SEQ ID NO: 156, one of the sequences set forth in SEQ ID NOs: 164-202, or one of the sequences set forth in 427-457.

In some examples, the first ABD includes an LCVD comprising the sequence of SEQ ID NO: 219, and an HCVD comprising the sequence of SEQ ID NO: 156, one of the sequences set forth in SEQ ID NOs: 164-202, or one of the sequences set forth in 427457.

In some examples, the first ABD includes an LCVD comprising the sequence of SEQ ID NO: 220, and an HCVD comprising the sequence of SEQ ID NO: 156, one of the sequences set forth in SEQ ID NOs: 164-202, or one of the sequences set forth in 427457.

In some examples, the first ABD includes an LCVD comprising the sequence of SEQ ID NO: 221, and an HCVD comprising the sequence of SEQ ID NO: 156, one of the sequences set forth in SEQ ID NOs: 164-202, or one of the sequences set forth in 427457.

In some examples, the first ABD includes an LCVD comprising the sequence of SEQ ID NO: 222, and an HCVD comprising the sequence of SEQ ID NO: 156, one of the sequences set forth in SEQ ID NOs: 164-202, or one of the sequences set forth in 427-457.

In some examples, the first ABD includes an LCVD comprising the sequence of SEQ ID NO: 223, and an HCVD comprising the sequence of SEQ ID NO: 156, one of the sequences set forth in SEQ ID NOs: 164-202, or one of the sequences set forth in 427457.

In some examples, the first ABD includes an LCVD comprising the sequence of SEQ ID NO: 224, and an HCVD comprising the sequence of SEQ ID NO: 156, one of the sequences set forth in SEQ ID NOs: 164-202, or one of the sequences set forth in 427457.

In some examples, the first ABD includes an LCVD comprising the sequence of SEQ ID NO: 225, and an HCVD comprising the sequence of SEQ ID NO: 156, one of the sequences set forth in SEQ ID NOs: 164-202, or one of the sequences set forth in 427457.

In some examples, the first ABD includes an LCVD comprising the sequence of SEQ ID NO: 226, and an HCVD comprising the sequence of SEQ ID NO: 156, one of the sequences set forth in SEQ ID NOs: 164-202, or one of the sequences set forth in 427457.

In some examples, the first ABD includes an LCVD comprising the sequence of SEQ ID NO: 227, and an HCVD comprising the sequence of SEQ ID NO: 156, one of the sequences set forth in SEQ ID NOs: 164-202, or one of the sequences set forth in 427457.

In some examples, the first ABD includes an LCVD comprising the sequence of SEQ ID NO: 228, and an HCVD comprising the sequence of SEQ ID NO: 156, one of the sequences set forth in SEQ ID NOs: 164-202, or one of the sequences set forth in 427-457.

In some examples, the first ABD includes an LCVD comprising the sequence of SEQ ID NO: 229, and an HCVD comprising the sequence of SEQ ID NO: 156, one of the sequences set forth in SEQ ID NOs: 164-202, or one of the sequences set forth in 427457.

In some examples, the first ABD includes an LCVD comprising the sequence of SEQ ID NO: 230, and an HCVD comprising the sequence of SEQ ID NO: 156, one of the sequences set forth in SEQ ID NOs: 164-202, or one of the sequences set forth in 427457.

In some examples, the first ABD includes an LCVD comprising the sequence of SEQ ID NO: 231, and an HCVD comprising the sequence of SEQ ID NO: 156, one of the sequences set forth in SEQ ID NOs: 164-202, or one of the sequences set forth in 427457.

In some examples, the first ABD includes an LCVD comprising the sequence of SEQ ID NO: 232, and an HCVD comprising the sequence of SEQ ID NO: 156, one of the sequences set forth in SEQ ID NOs: 164-202, or one of the sequences set forth in 427-457.

In some examples, the first ABD includes an LCVD comprising the sequence of SEQ ID NO: 233, and an HCVD comprising the sequence of SEQ ID NO: 156, one of the sequences set forth in SEQ ID NOs: 164-202, or one of the sequences set forth in 427457.

In some examples, the first ABD includes an LCVD comprising the sequence of SEQ ID NO: 458, and an HCVD comprising the sequence of SEQ ID NO: 156, one of the sequences set forth in SEQ ID NOs: 164-202, or one of the sequences set forth in 427457.

In some examples, the first ABD includes an LCVD comprising the sequence of SEQ ID NO: 459, and an HCVD comprising the sequence of SEQ ID NO: 156, one of the sequences set forth in SEQ ID NOs: 164-202, or one of the sequences set forth in 427457.

In some examples, the first ABD includes an LCVD comprising the sequence of SEQ ID NO: 460, and an HCVD comprising the sequence of SEQ ID NO: 156, one of the sequences set forth in SEQ ID NOs: 164-202, or one of the sequences set forth in 427457.

In some examples, the first ABD includes an LCVD comprising the sequence of SEQ ID NO: 461, and an HCVD comprising the sequence of SEQ ID NO: 156, one of the sequences set forth in SEQ ID NOs: 164-202, or one of the sequences set forth in 427457.

In some examples, the first ABD includes an LCVD comprising the sequence of SEQ ID NO: 462, and an HCVD comprising the sequence of SEQ ID NO: 156, one of the sequences set forth in SEQ ID NOs: 164-202, or one of the sequences set forth in 427-457.

In some examples, the first ABD includes an LCVD comprising the sequence of SEQ ID NO: 463, and an HCVD comprising the sequence of SEQ ID NO: 156, one of the sequences set forth in SEQ ID NOs: 164-202, or one of the sequences set forth in 427457.

In some examples, the first ABD includes an LCVD comprising the sequence of SEQ ID NO: 464, and an HCVD comprising the sequence of SEQ ID NO: 156, one of the sequences set forth in SEQ ID NOs: 164-202, or one of the sequences set forth in 427457.

In some examples, the first ABD includes an LCVD comprising the sequence of SEQ ID NO: 465, and an HCVD comprising the sequence of SEQ ID NO: 156, one of the sequences set forth in SEQ ID NOs: 164-202, or one of the sequences set forth in 427457.

In some examples, the first ABD includes an LCVD comprising the sequence of SEQ ID NO: 466, and an HCVD comprising the sequence of SEQ ID NO: 156, one of the sequences set forth in SEQ ID NOs: 164-202, or one of the sequences set forth in 427-457.

In some examples, the first ABD includes an LCVD comprising the sequence of SEQ ID NO: 467, and an HCVD comprising the sequence of SEQ ID NO: 156, one of the sequences set forth in SEQ ID NOs: 164-202, or one of the sequences set forth in 427457.

In some examples, the first ABD includes an LCVD comprising the sequence of SEQ ID NO: 468, and an HCVD comprising the sequence of SEQ ID NO: 156, one of the sequences set forth in SEQ ID NOs: 164-202, or one of the sequences set forth in 427457.

In some examples, the first ABD includes an LCVD comprising the sequence of SEQ ID NO: 469, and an HCVD comprising the sequence of SEQ ID NO: 156, one of the sequences set forth in SEQ ID NOs: 164-202, or one of the sequences set forth in 427457.

In some examples, the first ABD includes an LCVD comprising the sequence of SEQ ID NO: 470, and an HCVD comprising the sequence of SEQ ID NO: 156, one of the sequences set forth in SEQ ID NOs: 164-202, or one of the sequences set forth in 427457.

In some examples, the first ABD includes an LCVD comprising the sequence of SEQ ID NO: 471, and an HCVD comprising the sequence of SEQ ID NO: 156, one of the sequences set forth in SEQ ID NOs: 164-202, or one of the sequences set forth in 427457.

In some examples, the first ABD includes an LCVD comprising the sequence of SEQ ID NO: 472, and an HCVD comprising the sequence of SEQ ID NO: 156, one of the sequences set forth in SEQ ID NOs: 164-202, or one of the sequences set forth in 427-457.

In some examples, the first ABD includes an LCVD comprising the sequence of SEQ ID NO: 473, and an HCVD comprising the sequence of SEQ ID NO: 156, one of the sequences set forth in SEQ ID NOs: 164-202, or one of the sequences set forth in 427457.

In some examples, the first ABD includes an LCVD comprising the sequence of SEQ ID NO: 474, and an HCVD comprising the sequence of SEQ ID NO: 156, one of the sequences set forth in SEQ ID NOs: 164-202, or one of the sequences set forth in 427457.

In some examples, the first ABD includes an LCVD comprising the sequence of SEQ ID NO: 475, and an HCVD comprising the sequence of SEQ ID NO: 156, one of the sequences set forth in SEQ ID NOs: 164-202, or one of the sequences set forth in 427457.

In some examples, the first ABD includes an LCVD comprising the sequence of SEQ ID NO: 476, and an HCVD comprising the sequence of SEQ ID NO: 156, one of the sequences set forth in SEQ ID NOs: 164-202, or one of the sequences set forth in 427-457.

In some examples, the first ABD includes an LCVD comprising the sequence of SEQ ID NO: 477, and an HCVD comprising the sequence of SEQ ID NO: 156, one of the sequences set forth in SEQ ID NOs: 164-202, or one of the sequences set forth in 427457.

In some examples, the first ABD includes an LCVD comprising the sequence of SEQ ID NO: 478, and an HCVD comprising the sequence of SEQ ID NO: 156, one of the sequences set forth in SEQ ID NOs: 164-202, or one of the sequences set forth in 427457.

In some examples, the first ABD includes an LCVD comprising the sequence of SEQ ID NO: 479, and an HCVD comprising the sequence of SEQ ID NO: 156, one of the sequences set forth in SEQ ID NOs: 164-202, or one of the sequences set forth in 427457.

In some examples, the first ABD includes an LCVD comprising the sequence of SEQ ID NO: 480, and an HCVD comprising the sequence of SEQ ID NO: 156, one of the sequences set forth in SEQ ID NOs: 164-202, or one of the sequences set forth in 427457.

In some examples, the first ABD includes an LCVD comprising the sequence of SEQ ID NO: 481, and an HCVD comprising the sequence of SEQ ID NO: 156, one of the sequences set forth in SEQ ID NOs: 164-202, or one of the sequences set forth in 427457.

In some examples, the first ABD includes an LCVD comprising the sequence of SEQ ID NO: 482, and an HCVD comprising the sequence of SEQ ID NO: 156, one of the sequences set forth in SEQ ID NOs: 164-202, or one of the sequences set forth in 427-457.

In some examples, the first ABD includes an LCVD comprising the sequence of SEQ ID NO: 483, and an HCVD comprising the sequence of SEQ ID NO: 156, one of the sequences set forth in SEQ ID NOs: 164-202, or one of the sequences set forth in 427457.

In some examples, the first ABD includes an LCVD comprising the sequence of SEQ ID NO: 484, and an HCVD comprising the sequence of SEQ ID NO: 156, one of the sequences set forth in SEQ ID NOs: 164-202, or one of the sequences set forth in 427457.

In some examples, the first ABD includes an LCVD comprising the sequence of SEQ ID NO: 485, and an HCVD comprising the sequence of SEQ ID NO: 156, one of the sequences set forth in SEQ ID NOs: 164-202, or one of the sequences set forth in 427457.

In some examples, the first ABD includes an LCVD comprising the sequence of SEQ ID NO: 486, and an HCVD comprising the sequence of SEQ ID NO: 156, one of the sequences set forth in SEQ ID NOs: 164-202, or one of the sequences set forth in 427-457.

In some examples, the first ABD includes an LCVD comprising the sequence of SEQ ID NO: 487, and an HCVD comprising the sequence of SEQ ID NO: 156, one of the sequences set forth in SEQ ID NOs: 164-202, or one of the sequences set forth in 427457.

In some examples, the first ABD includes an LCVD comprising the sequence of SEQ ID NO: 488, and an HCVD comprising the sequence of SEQ ID NO: 156, one of the sequences set forth in SEQ ID NOs: 164-202, or one of the sequences set forth in 427457.

In some examples, the first ABD includes an LCVD comprising the sequence of SEQ ID NO: 489, and an HCVD comprising the sequence of SEQ ID NO: 156, one of the sequences set forth in SEQ ID NOs: 164-202, or one of the sequences set forth in 427457.

In some examples, the first ABD includes an LCVD comprising the sequence of SEQ ID NO: 490, and an HCVD comprising the sequence of SEQ ID NO: 156, one of the sequences set forth in SEQ ID NOs: 164-202, or one of the sequences set forth in 427457.

In some examples, the first ABD includes an LCVD comprising the sequence of SEQ ID NO: 491, and an HCVD comprising the sequence of SEQ ID NO: 156, one of the sequences set forth in SEQ ID NOs: 164-202, or one of the sequences set forth in 427457.

In some examples, the first ABD includes an LCVD comprising the sequence of SEQ ID NO: 492, and an HCVD comprising the sequence of SEQ ID NO: 156, one of the sequences set forth in SEQ ID NOs: 164-202, or one of the sequences set forth in 427-457.

In some examples, the first ABD includes an HCVD of A1,2X4 with one or more amino acids substituted with a histidine. In some examples, the first ABD includes an LCVD of A1,2X4 with one or more amino acids substituted with a histidine. In some examples, the first ABD includes an HCVD of A1,2X4 with one or more amino acids substituted with a histidine; and an LCVD of A1,2X4 with one or more amino acids substituted with a histidine. In some examples, the HCVD of A1,2X4 comprises SEQ ID NO: 234. In some examples, the LCVD of A1,2X4 comprises SEQ ID NO: 235. In each of the foregoing examples, an alanine may be substituted where a histidine was already present.

In some examples, the first ABD comprises an HCVD comprising a CDR1, a CDR2, and a CDR3 of SEQ ID NOs: 236, 237, and 238, respectively, with collectively a total of one or more amino acid positions in SEQ ID NOs: 236, 237, and 238 substituted with a histidine. In some examples, the first ABD comprises an LCVD comprising a CDR1, a CDR2, and a CDR3 of SEQ ID NOs: 239, 240, and 241, respectively, with collectively a total of one or more amino acid positions in SEQ ID NOs: 239, 240, and 241 substituted with a histidine. In some examples, the first ABD includes: an HCVD comprising a CDR1, a CDR2, and a CDR3 of SEQ ID NOs: 236, 237, and 238, respectively, with collectively a total of one or more amino acid positions in NOs: 236, 237, and 238 substituted with a histidine; and an LCVD comprising a CDR1, a CDR2, and a CDR3 of SEQ ID NOs: 239, 240, and 241, respectively, with collectively a total of one or more amino acid positions in SEQ ID NOs: 239, 240, and 241 substituted with a histidine. In each of the foregoing examples, an alanine may be substituted where a histidine was already present.

In some examples, the first ABD includes an HCVD that is at least 90%, 95%, or 98% identical to SEQ ID NO: 234, where the HCVD includes a histidine at one or more amino acid positions in SEQ ID NO: 234. In some examples, the first ABD includes an LCVD that is at least 90%, 95%, or 98% identical to SEQ ID NO: 235, where the LCVD includes a histidine at one or more amino acid positions in SEQ ID NO: 235. In some examples, the first ABD includes an HCVD that is at least 90%, 95%, or 98% identical to SEQ ID NO: 234, where the HCVD includes a histidine at one or more amino acid positions in SEQ ID NO: 234, and an LCVD that is at least 90%, 95%, or 98% identical to SEQ ID NO: 235, where the LCVD includes a histidine at one or more amino acid positions in SEQ ID NO: 235. In each of the foregoing examples, an alanine may be substituted where a histidine was already present.

Also provided herein are pharmaceutical compositions including any of the ABPCs described herein. Also provided herein are methods of treating a subject in need thereof that include administering a therapeutically effective amount of any of the ABPCs described herein to the subject.

In some examples, a composition including the ABPC (e.g., any of the ABPCs described herein) can provide for an increase (e.g., a detectable increase) (e.g., any of the percents increase or ranges of percents increase recited in WO 2021/022039 at pages 389-404 (or recited in the corresponding published application US 2022/0281984), which are incorporated by reference herein in their entirety) in toxin liberation in the target mammalian cell (e.g., any of the target mammalian cells described herein) as compared to a composition including the same amount of a control ABPC (e.g., any of the exemplary control ABPCs described herein).

In some examples, a composition including the ABPC (e.g., any of the ABPCs described herein) can provide for an increase (e.g., a detectable increase) (e.g., any of the folds increase or ranges of folds increase recited in WO 2021/022039 at pages 404-420 (or recited in the corresponding published application US 2022/0281984), which are incorporated by reference herein in their entirety) in toxin liberation in the target mammalian cell (e.g., any of the target mammalian cells described herein) as compared to a composition including the same amount of a control ABPC (e.g., any of the exemplary control ABPCs described herein).

In some examples, a composition including the ABPC (e.g., any of the ABPCs described herein) can provide for an increase (e.g., a detectable increase) (e.g., at least a 1%, 2%, 5%, 10%, 15%, 20%, 25%, 30%, 35%, 40%, 45%, 50%, 55%, 60%, 65%, 70%, 75%, 80%, 85%, 90%, 95%, 100%, 120%, 140%, 160%, 180%, 200%, 250%, 300%, 350%, 400%, 450%, 500%, 1,000%, 2,000%, 3,000%, 4,000%, 5,000%, 6,000%, 7,000%, 8,000%, 9,000%, or a 10,000% increase, or about a 1% to 10,000% increase (e.g., or any of the subranges)) in target mammalian cell killing as compared to a composition including the same amount of a control ABPC.

In some examples, a composition including the ABPC (e.g., any of the ABPCs described herein) can provide for an increase (e.g., a detectable increase) (e.g., at least a 0.1-, 0.2-, 0.3-, 0.4-, 0.5-, 0.6-, 0.7-, 0.8-, 0.9-, 1.0-, 1.2-, 1.4-, 1.5-, 1.6-, 1.8-, 2.0-, 2.2-, 2.4-, 2.5-, 2.6-, 2.8-, 3.0-, 3.5-, 4.0-, 4.5-, 5.0-, 5.5-, 6.0-, 6.5-, 7.0-, 7.5-, 8.0-, 8.5-, 9.0-, 9.5-, 10-, 15-, 20-, 25-, 30-, 35-, 40-, 40-, 45-, 50-, 55-, 60-, 65-, 70-, 80-, 85-, 90-, 95-fold increase, or at least a 100-fold increase, or a 0.1-100-fold increase in target mammalian cell killing as compared to a composition including the same amount of a control ABPC

In some examples, a composition including any of the ABPCs described herein (e.g., upon contacting target mammalian cells presenting TROP2 on their surface) results in decreased (e.g., ≥a 1%, 5%, 10%, 15%, 20%, 25%, 30%, 35%, 40%, 45%, 50%, 55%, 60%, 65%, 70%, 75%, 80%, 85%, 90%, 95% decrease, or at least a 99% decrease, about a 1%-99% decrease, or any of the subranges of this range described herein) IC₅₀ (for target mammalian cell killing) as compared to the IC₅₀ for a composition including the same amount of a control ABPC.

In some examples, a composition including any of the ABPCs described herein (e.g., upon contacting target mammalian cells presenting TROP2 on their surface) can provide for an increase (e.g., at least a 0.1-, 0.2-, 0.4-, 0.6-, 0.8-, 1-, 2-, 5-, 10-, 15-, 20-, 25-, 30-, 35-, 40-, 45-, 50-, 55-, 60-, 65-, 70-, 75-, 80-, 85-, 90-, 95-fold increase, or at least a 100-fold increase, or about a 0.1-500-fold increase (or any of the subranges of this range described herein) in the ratio of K_{D} on target mammalian cells presenting TROP2 on their surface at a neutral pH to IC₅₀ at the neutral pH on the same target cells, e.g., as compared to a control ABPC.

In some examples, a composition including the ABPC (e.g., any of the ABPCs described herein) can provide for an increase (e.g., a detectable increase) (e.g., at least a 1%, 2%, 5%, 10%, 15%, 20%, 25%, 30%, 35%, 40%, 45%, 50%, 55%, 60%, 65%, 70%, 75%, 80%, 85%, 90%, 95%, 100%, 120%, 140%, 160%, 180%, 200%, 250%, 300%, 350%, 400%, 450%, 500%, 1,000%, 2,000%, 3,000%, 4,000%, 5,000%, 6,000%, 7,000%, 8,000%, 9,000% increase, or at least a 10,000% increase, or about a 1%-10,000% increase (e.g., or any of the subranges of this range described herein)) in endolysosomal delivery in the target mammalian cell as compared to a composition including the same amount of a control ABPC

In some examples, a composition including the ABPC can provide for an increase (e.g., ≥a 0.1-, 0.2-, 0.3-, 0.4-, 0.5-, 0.6-, 0.7-, 0.8-, 0.9-, 1.0-, 1.2-, 1.4-, 1.5-, 1.6-, 1.8-, 2.0-, 2.2-, 2.4-, 2.5-, 2.6-, 2.8-, 3.0-, 3.5-, 4.0-, 4.5-, 5.0-, 5.5-, 6.0-, 6.5-, 7.0-, 7.5-, 8.0-, 8.5-, 9.0-, 9.5-, 10-, 15-, 20-, 25-, 30-, 35-, 40-, 45-, 50-, 55-, 60-, 65-, 70-, 75-, 80-, 85-, 90-, 95-fold increase, or at least a 100-fold increase, or about a 0.1-100-fold increase in endolysosomal delivery in the target mammalian cell as compared to a composition including the same amount of a control ABPC.

In examples of any of the ABPCs described herein, the target mammalian cell does not express an FcRn receptor, or expresses a lower (e.g., a detectably lower) level (e.g., at least a 1% decreased, at least a 2%, 5%, 10% decrease, at least a 15%, 20%, 25%, 30%, 35%, 40%, 45%, 50%, 55%, 60%, 65%, 70%, 75%, 80%, 85%, 90%, 95% decreased, or at least a 99% decreased level) of FcRn receptor as compared to a FcRn expressing control cell (e.g., HUVEC-ThermoFisher #C0035C). In some examples, the target mammalian cell is a cancer cell. In some examples, the ABPC is cytotoxic or cytostatic to the target mammalian cell.

In some examples, a composition including any of the ABPCs described herein (e.g., upon administration to a subject) results in less (e.g., a 1% decrease to about a 99% decrease, or any of the subranges of this range described herein) of a reduction in the level of TROP2 presented on the surface of the target cell as compared to a composition including the same amount of a control ABPC (e.g., any of the control ABPCs described herein). In some examples, the composition does not result in a detectable reduction in the level of the TROP2 presented on the surface of the target mammalian cell.

In some examples, the ABPC is cross-reactive with a non-human primate TROP2 and a human TROP2. In some examples, the ABPC is cross-reactive with a non-human primate TROP2, a human TROP2, and one or both of rat TROP2 and a mouse TROP2. In some examples, the ABPC is cross-reactive with a non-human primate TROP2, a human TROP2, a rat TROP2, and a mouse TROP2. In some examples, the ABPC is cross-reactive with mouse TROP2 and rat TROP2. In some examples, the ABD binds to an epitope of TROP2 that is present on the surface of cells from an Old World Monkey.

Some examples of any of the ABPCs described herein can further include a second ABD (e.g., any of the exemplary ABDs described herein). Non-limiting aspects of these methods are described below, and can be used in any combination without limitation. Additional aspects of these methods are known in the art.

### TROP2 or Epitope of TROP2

Multiple TROP2-binding monoclonal antibodies have been described in the literature and can be used as a template for engineering pH-dependent binding. The amino acid sequence of the mature Human TROP2 can be found in SEQ ID NO: 2. The amino acid sequence of the extracellular domain of TROP2 can be found in SEQ ID NO: 3.

### Antigen-Binding Protein Constructs

Any of the antigen-binding protein constructs (ABPCs) described herein can be a single polypeptide, or can include 2, 3, 4, 5, 6, 7, 8, 9, or 10 (the same or different) polypeptides. In some embodiments where the ABPC is a single polypeptide, the ABPC can include a single ABD or two ABDs. In some embodiments where the ABPC is a single polypeptide and includes two ABDs, the first and second ABDs can be identical or different from each other (and can specifically bind to the same or different antigens or epitopes).

In some embodiments where the ABPC is a single polypeptide, the first ABD and the second ABD (if present) can each be independently selected from the group of: a VH domain, a VHH domain, a VNAR domain, and a scFv. In some embodiments where the ABPC is a single polypeptide, the antigen-binding protein construct can be a BiTe, a (scFv)2, a nanobody, a nanobody-HSA, a DART, a TandAb, a scDiabody, a scDiabody-CH3, scFv-CH-CL-scFv, a HSAbody, scDiabody-HAS, a tandem-scFv, an Adnectin, a DARPin, a fibronectin, a DEP conjugate, a PROTAB, or a PROTAC. Additional examples of ABDs that can be used when the ABPC is a single polypeptide are known in the art

A VHH domain is a single monomeric variable antibody domain that can be found in camelids. A VNAR domain is a single monomeric variable antibody domain that can be found in cartilaginous fish. Non-limiting aspects of V_{H}H domains and V_{NAR} domains are described in, e.g., Cromie et al., Curr. Top. Med. Chem. 15:2543-2557, 2016; De Genst et al., Dev. Comp. Immunol. 30:187-198, 2006; De Meyer et al., Trends Biotechnol. 32:263-270, 2014; Kijanka et al., Nanomedicine 10:161-174, 2015; Kovaleva et al., Expert. Opin. Biol. Ther. 14:1527-1539, 2014; Krah et al., Immunopharmacol. Immunotoxicol. 38:21-28, 2016; Mujic-Delic et al., Trends Pharmacol. Sci. 35:247-255, 2014; Muyldermans, J. Biotechnol. 74:277-302, 2001; Muyldermans et al., Trends Biochem. Sci. 26:230-235, 2001; Muyldermans, Ann. Rev. Biochem. 82:775-797, 2013; Rahbarizadeh et al., Immunol. Invest. 40:299-338, 2011; Van Audenhove et al., ELBioMedicine 8:40-48, 2016; Van Bockstaele et al., Curr. Opin. Investig. Drugs 10:1212-1224, 2009; Vincke et al., Methods Mol. Biol. 911:15-26, 2012; and Wesolowski et al., Med. Microbiol. Immunol. 198:157-174, 2009.

In some embodiments where the ABPC is a single polypeptide and includes two ABDs, the first ABD and the second ABD can both be VHH domains, or at least one ABD can be a VHH domain. In some embodiments where the ABPC is a single polypeptide and includes two ABDs, the first ABD and the second ABD are both VNAR domains, or at least one ABD is a V_{NAR} domain. In some embodiments where the ABPC is a single polypeptide, the first ABD is a scFv domain. In some embodiments where the ABPC is a single polypeptide and includes two ABDs, the first ABD and the second ABD can both be scFv domains, or at least one ABD can be a scFv domain.

In some embodiments, the ABPC can include two or more polypeptides (e.g., 2, 3, 4, 5, 6, 7, 8, 9, or 10 polypeptides). In some embodiments where the ABPC includes two or more polypeptides, two, three, four, five or six of the polypeptides of the two or more polypeptides can be identical.

In some embodiments where the ABPC includes two or more polypeptides (e.g., 2, 3, 4, 5, 6, 7, 8, 9, or 10 polypeptides), two or more of the polypeptides of the ABPC can assemble (e.g., non-covalently assemble) to form one or more ABDs, e.g., an antigen-binding fragment of an antibody (e.g., any of the antigen-binding fragments of an antibody described herein), a VHH-scAb, a VHH-Fab, a Dual scFab, a F(ab′)2, a diabody, a crossMab, a DAF (two-in-one), a DAF (four-in-one), a DutaMab, a DT-IgG, a knobs-in-holes common light chain, a knobs-in-holes assembly, a charge pair, a Fab-arm exchange, a SEEDbody, a LUZ-Y, a Fcab, a x-body, an orthogonal Fab, a DVD-IgG, a IgG(H)-scFv, a scFv-(H)IgG, IgG(L)-scFv, scFv-(L)IgG, IgG(L,H)-Fv, IgG(H)-V, V(H)—IgG, IgG(L)-V, V(L)-IgG, KIH IgG-scFab, 2scFv-IgG, IgG-2scFv, scFv4-Ig, Zybody, DVI-IgG, Diabody-CH3, a triple body, a miniantibody, a minibody, a TriBi minibody, scFv-CH3 KIH, Fab-scFv, a F(ab′)2-scFv2, a scFv-KIH, a Fab-scFv-Fc, a tetravalent HCAb, a scDiabody-Fc, a Diabody-Fc, a tandem scFv-Fc, a VHH-Fc, a tandem VHH-Fc, a VHH-Fc KiH, a Fab-VHH-Fc, an Intrabody, a dock and lock, an ImmTAC, an IgG-IgG conjugate, a Cov-X-Body, a scFv1-PEG-scFv2, an Adnectin, a DARPin, a fibronectin, a DEP conjugate, a PROTAB, or a PROTAC. See, e.g., Spiess et al., Mol. Immunol. 67:95-106, 2015, incorporated in its entirety herewith, for a description of these elements. Non-limiting examples of an antigen-binding fragment of an antibody include an Fv fragment, a Fab fragment, a F(ab′)2 fragment, and a Fab′ fragment. Additional examples of an antigen-binding fragment of an antibody is an antigen-binding fragment of an IgG (e.g., an antigen-binding fragment of IgG1, IgG2, IgG3, or IgG4) (e.g., an antigen-binding fragment of a human or humanized IgG, e.g., human or humanized IgG1, IgG2, IgG3, or IgG4); an antigen-binding fragment of an IgA (e.g., an antigen-binding fragment of IgAQ1 or IgA2) (e.g., an antigen-binding fragment of a human or humanized IgA, e.g., a human or humanized IgAQ1 or IgA2); an antigen-binding fragment of an IgD (e.g., an antigen-binding fragment of a human or humanized IgD); an antigen-binding fragment of an IgE (e.g., an antigen-binding fragment of a human or humanized IgE); or an antigen-binding fragment of an IgM (e.g., an antigen-binding fragment of a human or humanized IgM).

A “Fv” fragment includes a non-covalently-linked dimer of one HCVD and one LCVD.

A “Fab” fragment includes, the constant domain of the light chain and the first constant domain (C_{H1}) of the heavy chain, in addition to the heavy and LCVDs of the Fv fragment.

A “F(ab′)₂” fragment includes two Fab fragments joined, near the hinge region, by disulfide bonds.

A “dual variable domain immunoglobulin” or “DVD-Ig” refers to multivalent and multispecific binding proteins as described, e.g., in DiGiammarino et al., Methods Mol. Biol. 899:145-156, 2012; Jakob et al., MABs 5:358-363, 2013, and U.S. Pat. Nos. 7,612,181; 8,258,268; 8,586,714; 8,716,450; 8,722,855; 8,735,546; and 8,822,645, each of which is incorporated by reference in its entirety.

DARTs are described in, e.g., Garber, Nature Reviews Drug Discovery 13:799-801, 2014.

Additional aspects of ABPCs are known in the art.

### Antigen-Binding Domains

In some embodiments of any of the antigen-binding protein constructs (ABPCs) described herein, the dissociation rate of the first ABD (and optionally the second ABD, if present) at a pH of about 4.0 to about 6.5 (e.g., about 4.0 to about 6.4, or any of the ranges of pH recited in WO 2021/022039 at pages 428-430 (or recited in the corresponding published application US 2022/0281984), which are incorporated by reference herein in their entirety) is faster (e.g., at least 5% faster, or any of the % faster or ranges of % faster recited in WO 2021/022039 at pages 430-447 (or recited in the corresponding published application US 2022/0281984), which are incorporated by reference herein in their entirety) than the dissociation rate at a pH of about 7.0 to about 8.0 (e.g., about 7.0-7.9, 7.0-7.8, 7.0-7.7, 7.0-7.6, 7.0-7.5, 7.0-7.4, 7.0-7.3, 7.0-7.2, 7.0-7.1, 7.1-8.0, 7.1-7.9, 7.1-7.8, 7.1-7.7, 7.1-7.6, 7.1-7.5, 7.1-7.4, 7.1-7.3, 7.1-7.2, 7.2-8.0, 7.2-7.9, 7.2-7.8, 7.2-7.7, 7.2-7.6, 7.2-7.5, 7.2-7.4, 7.2-7.3, 7.3-8.0, 7.3-7.9, 7.3-7.8, 7.3-7.7, 7.3-7.6, 7.3-7.5, 7.3-7.4, 7.4-8.0, 7.4-7.9, 7.4-7.8, 7.4-7.7, 7.4-7.6, 7.4-7.5, 7.5-8.0, 7.5-7.9, 7.5-7.8, 7.5-7.7, 7.5-7.6, 7.5-8.0, 7.6-7.9, 7.6-7.8, 7.6-7.7, 7.7-8.0, 7.7-7.9, 7.7-7.8, 7.8-8.0, 7.8-7.9, or about 7.9-8.0).

In some embodiments of any of the ABPCs described herein, the dissociation constant (K_{D}) of the first ABD (and optionally the second ABD, if present) at a pH of about 4.0 to about 6.5 (e.g., any of the subranges of this range described herein) is greater (e.g., detectably greater) (e.g., at least about 5, 10, 15, 20, 25, 30, 35, 40, 45, 50, 55, 60, 65, 70, 80, 85, 90, 95, 100, 120, 140, 160, 180, 200, 220, 240, 260, 280, 300, 320, 340, 360, 380, 400, 420, 440, 460, 480, 500, 1,000, 2,000, 3,000, 4,000, 5,000, 6,000, 7,000, 8,000, 9,000, or at least about 10,000% greater, or about 5% to about 10,000% greater, or any of the ranges of percents greater K_{D} recited in WO 2021/022039 at pages 448-466 (or recited in the corresponding published application US 2022/0281984), which are incorporated by reference herein in their entirety) than the K_{D} at a pH of about 7.0 to about 8.0 (e.g., any of the subranges of this range described herein).

In some embodiments of any of the antigen-binding protein constructs (ABPCs) described herein, the dissociation rate of the first ABD (and optionally the second ABD, if present) at a pH of about 4.0 to about 6.5 (e.g., any of the subranges of this range described herein) is faster (e.g., at least 0.2-fold faster, or any of the folds faster or ranges of folds faster recited in WO 2021/022039 at pages 466-468 (or recited in the corresponding published application US 2022/0281984), which are which are incorporated by reference herein in their entirety) than the dissociation rate at a pH of ~7.0 to ~8.0 (e.g., or any of the subranges).

In some embodiments of any of the antigen-binding protein constructs (ABPCs) described herein, the dissociation constant (K_{D}) of the first ABD (and optionally the second ABD, if present) at a pH of about 4.0 to about 6.5 (e.g., any of the subranges of this range described herein) is greater (e.g., detectably greater) (e.g., at least 0.2-fold greater, or any of the fold greater or ranges of fold greater recited in WO 2021/022039 at pages 468-470 (or recited in the corresponding published application US 2022/0281984), which are which are incorporated by reference herein in their entirety), than the K_{D} at a pH of about 7.0 to about 8.0 (e.g., any of the subranges of this range described herein).

In some embodiments of the ABPCs that include a first ABD and a second ABD, the first and second ABDs are identical or are at least 80% identical (e.g., ≥82%, ≥84%, ≥86%, ≥88%, ≥90%, ≥91%, ≥92%, ≥93%, ≥94%, ≥95%, ≥96%, ≥97%, ≥98%, or ≥99% identical) in amino acid sequence to each other. In some embodiments, the ABPCs that include a first ABD and a second ABD, the first ABD and the second ABD have a sequence that is less than 80% identical (e.g., <75%, <70%, <65%, <60%, <55%, <50%, <45%, <40%, <35%, <30%, <25%, <20%, <15%, <10%, or <5% identical) to each other. In some embodiments of ABPCs that include a first and a second ABD, the first and second ABD binds two different epitopes (e.g., two different epitopes on TROP2 or the first ABD binding specifically to TROP2 and the second ABD binding to an antigen other than TROP2).

In some embodiments of any of the ABPCs described herein, the K_{D} of the first ABD (and optionally, the second ABD if present) at a pH of about 7.0-8.0 (e.g., any of the subranges of this range described herein) is between about 1 μM-5 μM (e.g., 1 μM-2 μM, or any of the ranges of K_{D} recited in WO 2021/022039 at pages 471-492 (or recited in published application US 2022/0281984), which are herein incorporated by reference in their entirety).

In some embodiments of any of the ABPCs described herein, the K_{D} of the first ABD (and optionally, the second ABD, if present) at a pH of about 4.0-6.5 (e.g., any of the subranges of this range described herein) can be greater than 1 nM (e.g., between about 1 nM-1 mM, or any of the ranges of K_{D} recited in WO 2021/022039 at pages 492-496 (or recited in published application US 2022/0281984), which are herein incorporated by reference in their entirety).

A variety of different methods known in the art can be used to determine the K_{D} values of any of the antigen-binding protein constructs described herein (e.g., an electrophoretic mobility shift assay, a filter binding assay, surface plasmon resonance, a biomolecular binding kinetics assay, in vitro binding assay on antigen-expressing cells, etc.).

In some examples, the half-life of the ABPC in vivo is decreased (e.g., a detectable decrease) (e.g., at least a 1% decrease, or any of the percents decrease or ranges of percents decrease recited in WO 2021/022039 at pages 496-500 (or recited in the corresponding published application US 2022/0281984), which are herein incorporated by reference in their entirety) as compared to the half-life of a control ABPC (e.g., any of the exemplary control ABPCs described herein). Conversely, in some examples, the half-life of the ABPC in vivo may be increased as compared to the half-life of a control ABPC.

### Conjugation

In some embodiments, the ABPCs provided herein can be conjugated to a drug (e.g., a chemotherapeutic drug, a small molecule), a toxin, or a radioisotope. Non-limiting examples of drugs, toxins, and radioisotopes (e.g., useful for the treatment of cancer) are known in the art.

In some embodiments, at least one polypeptide of any of the ABPCs described herein is conjugated to the toxin, the radioisotope, or the drug via a cleavable linker. In some embodiments, the cleavable linker includes a protease cleavage site. In some embodiments, the cleavable linker is cleaved on the ABPC once it is transported to the lysosome or late endosome by the target mammalian cell. In some embodiments, cleavage of the linker functionally activates the drug or toxin.

In some embodiments, at least one polypeptide of any of the ABPCs described herein is conjugated to the toxin, the radioisotope, or the drug via a non-cleavable linker. In some embodiments, the conjugated toxin, radioisotope, or drug is released during lysosomal and/or late endosomal degradation of the ABPC.

Non-limiting examples of cleavable linkers include: hydrazone linkers, peptide linkers, disulfide linkers, and thioether linkers. See, e.g., Carter et al., Cancer J. 14(3):154-169, 2008; Sanderson et al., Clin. Cancer Res. 11(2 Pt1):843-852, 2005; Chari et al., Acc. Chem. Res. 41(1); 98-107, 2008; Oflazoglu et al., Clin. Cancer Res. 14(19). 6171-6180, 2008; and Lu et al., Int. J. Mol. Sci. 17(4): 561, 2016.

Non-limiting examples of non-cleavable linkers include: maleimide alkane-linkers and maleimide cyclohexane linker (MMC) (see, e.g., McCombs et al., AAPS J. 17(2):339-351, 2015).

In some embodiments, any of the ABPCs described herein is cytotoxic or cytostatic to the target mammalian cell.

In some embodiments, the antibodies provided herein can comprise one or more amino acid substitutions to provide a conjugation site (e.g., conjugated to a drug, a toxin, a radioisotope). In some embodiments, the antibodies provided herein can have one conjugation site. In some embodiments, the antibodies described herein can have two conjugation sites. In some embodiments, the antibodies provided herein can have three or more conjugation sites.

Naturally-occurring cysteine amino acids can also provide conjugation. In some embodiments, the antibodies provided herein can have a drug, a toxin, or a radioisotope conjugated at one or more naturally-occurring conjugation sites. In some embodiments, the antibodies provided herein can have a drug, a toxin, or a radioisotope conjugated at one or more naturally occurring conjugation sites. In some embodiments, the antibodies provided herein can have a drug, a toxin, or a radioisotope conjugated at one or more naturally occurring conjugation sites and one or more engineered conjugation sites.

Conjugation through engineered cysteines is achieved by methods known in the art. Briefly, engineered cysteine-containing antibody is prepared for conjugation by treatment with a reducing agent, for example, tris (2-carboxyethyl) phosphine (TCEP), Dithiothreitol (DTT), or 2-Mercaptoethanol (BME). In the reduction reaction the reducing reagent with disulfide bonds in the antibody, breaking interchain disulfides and removing disulfide caps from the engineered cysteines. An optional reoxidation step, achieved by exposure of the solution to air, or an oxidizing agent such as dehydroascorbic acid, allows reformation of the interchain disulfide bonds, leaving the engineered cysteines with a thiolate reactive group. Conjugation with a maleimide functionality on the linker-payload, maleimide-vc-MMAE, is achieved by reaction with the payload in buffered solution, containing cosolvent such as ethanol, dimethylacetamide (DMA), or dimethyl sulfoxide (DMSO). The crude conjugated antibody solution is purified by size exclusion chromatography, or selective filtration methods, such as tangential flow filtration. In this step, residual unreacted payload, reducing agent and oxidizing agents are removed from the reaction mixture, and the conjugated ADC product may be transferred into a desirable formulation buffer.

Conjugation through hinge cysteines is achieved by similar methods, using antibodies with, or without, additional engineered cysteine conjugation sites. Briefly, the antibody is prepared for conjugation by treatment with a reducing agent, for example, tris (2-carboxyethyl) phosphine (TCEP) or Dithiothreitol (DTT). The reducing strength and concentration of the reducing agent are selected such that some or all of the interchain disulfide bonds are reduced leaving free cysteines for conjugation. The solution may be directly conjugated in the presence of excess reducing agent. Conjugation with a maleimide functionality on the linker-payload, maleimide-vc-MMAE, is achieved by reaction with the payload in buffered solution, containing cosolvent such as ethanol, dimethylacetamide (DMA), or dimethyl sulfoxide (DMSO). Unreacted linker-payload may be rendered non-reactive by addition of a sacrificial thiolate molecule such as acetyl-cysteine. The crude conjugated antibody solution may be further purified by methods known in the art, including hydrophobic interaction chromatography, ion-exchange chromatography, or mixed-mode chromatography such as ceramic hydroxyapatite chromatography. Isolation of chromatography fractions allows selection of the desired antibody to payload ratio and removal of unreacted antibody, protein aggregates and fragments, and payload-related reaction side products. The purified antibody drug conjugate may be further purified by size exclusion chromatography, or selective filtration methods, such as TFF. In this step the conjugated ADC product may also be transferred into a desirable formulation buffer.

In some examples, an antibody conjugate can be made comprising an antibody linked to monomethyl auristatin E (MMAE) via a valine-citrulline (vc) linker (hereafter, TROP2-IgG-DC). Conjugation of the antibody with vcMMAE begins with a partial reduction of the TROP2-IgG followed by reaction with maleimidocaproyl-Val-Cit-PABC-MMAE (vcMMAE). The TROP2-IgG (10 mg/mL) is partially reduced by addition of TCEP (molar equivalents of TCEP:mAb is 2:1) followed by incubation at 4° C. overnight. The reduction reaction is then warmed to 25° C. To conjugate all of the thiols, vcMMAE is added to a final vcMMAE:reduced Cys molar ratio of 1:10. The conjugation reaction is carried out in the presence of 10% v/v of DMA and allowed to proceed at 25° C. for 60 minutes.

In some examples, an antibody conjugate (ADC) is made comprising the TROP2-binding IgG (hereafter, TROP2-IgG) described herein linked to monomethyl auristatin E (MMAE) via a valine-citrulline (vc) linker (hereafter, TROP2-IgG-DC) Conjugation of the antibody with vcMMAE begins with a partial reduction of the TROP2-IgG followed by reaction with maleimidocaproyl-Val-Cit-PABC-MMAE (vcMMAE). The TROP2-IgG (10 mg/mL) is reduced by addition of DTT (molar equivalents of DTT:mAb is 100:1) followed by incubation at 25° C. overnight. The reduced TROP2-IgG (10 mg/mL) is then re-oxidized by exposure to DHAA (molar equivalents of DHAA:mAb is 10:1) followed by incubation at 25° C. for 2 hours. To conjugate all of the thiols, vcMMAE is added to a final vcMMAE:mAb molar ratio of 4:1. The conjugation reaction is carried out in the presence of 10% v/v of DMA and allowed to proceed at 25° C. for 3 hours.

In some examples, an ADC is made according to the methods referenced in Daisuke Okajima et al, “Datopotamab Deruxtecan, a Novel TROP2-directed Antibody-drug Conjugate, Demonstrates Potent Antitumor Activity by Efficient Drug Delivery to Tumor Cells”, Mol Cancer Ther. 2021 Dec. 1; 20(12): 2329-2340, and/or Chau C H et al, “Novel antibody drug conjugates containing exatecan derivative-based cytotoxic payloads”, Bioorg Med Chem Lett 2016; 26:1542-5, which are herein incorporated by reference in their entireties.

In some examples, antibodies were produced by transient transfection of CHO cells (0.5 L scale) with the corresponding heavy chain and light chain expression plasmids. Supernatants were harvested after 7 days of incubation at 37° C. and the antibodies were purified by ProA affinity chromatography followed by size exclusion chromatography (SEC) and formulated in 20 mM His, 150 mM NaCl, pH6. Purity and identity of antibodies were assessed by SDS-PAGE, SEC-HPLC, and LC-MS. Antibodies were partially reduced for 17 h at 22° C. with TCEP and 10% DMA cosolvent for maleimide based GGFG-DXd conjugation to hinge cysteines with an average drug to antibody ratio (DAR) of about 4. Conjugated ADCs were purified by filtration using Amicon spin columns, DAR was assessed by hydrophobic interaction chromatography (HIC), and free drug was assessed by LC-MS. For a review of conjugates, see e.g., Laurence Gauzy-Lazo et al., “Advances in Antibody-Drug Conjugate Design: Current Clinical Landscape and Future Innovations”, SLAS Discovery, 25(8), p 843-868, September 2020.

### Expression of an Antigen-Binding Protein Construct in a Cell

Also provided herein are methods of generating a recombinant cell that expresses an ABPC (e.g., any of the ABPCs described herein) that include: introducing into a cell a nucleic acid encoding the ABPC to produce a recombinant cell; and culturing the recombinant cell under conditions sufficient for the expression of the ABPC. In some embodiments, the introducing step includes introducing into a cell an expression vector including a nucleic acid encoding the ABPC to produce a recombinant cell.

Any of the ABPCs described herein can be produced by any cell, e.g., a eukaryotic cell or a prokaryotic cell. As used herein, the term “eukaryotic cell” refers to a cell having a distinct, membrane-bound nucleus. Such cells may include, for example, mammalian (e.g., rodent, non-human primate, or human), insect, fungal, or plant cells. In some embodiments, the eukaryotic cell is a yeast cell, such as Saccharomyces cerevisiae. In some embodiments, the eukaryotic cell is a higher eukaryote, such as mammalian, avian, plant, or insect cells. As used herein, the term “prokaryotic cell” refers to a cell that does not have a distinct, membrane-bound nucleus. In some embodiments, the prokaryotic cell is a bacterial cell.

Methods of culturing cells are well known in the art. Cells can be maintained in vitro under conditions that favor proliferation, differentiation, and growth. Briefly, cells can be cultured by contacting a cell (e.g., any cell) with a cell culture medium that includes the necessary growth factors and supplements to support cell viability and growth.

Methods of introducing nucleic acids and expression vectors into a cell (e.g., a eukaryotic cell) are known in the art. Non-limiting examples of methods that can be used to introduce a nucleic acid into a cell include lipofection, transfection, electroporation, microinjection, calcium phosphate transfection, dendrimer-based transfection, cationic polymer transfection, cell squeezing, sonoporation, optical transfection, impalection, hydrodynamic delivery, magnetofection, viral transduction (e.g., adenoviral and lentiviral transduction), and nanoparticle transfection.

Provided herein are methods that further include isolation of the ABPCs from a cell (e.g., a eukaryotic cell) using techniques well-known in the art (e.g., ammonium sulfate precipitation, polyethylene glycol precipitation, ion-exchange chromatography (anion or cation), chromatography based on hydrophobic interaction, metal-affinity chromatography, ligand-affinity chromatography, and size exclusion chromatography).

### Methods of Treatment

Provided herein are methods of treating a cancer characterized by having a population of cancer cells that have TROP2 or an epitope of TROP2 presented on their surface, which include: administering a therapeutically effective amount of any of the pharmaceutical compositions described herein or any of the ABPCs described herein to a subject identified as having a cancer characterized by having the population of cancer cells.

Also provided herein are methods of reducing the volume of a tumor in a subject, wherein the tumor is characterized by having a population of cancer cells that have TROP2 or an epitope of TROP2 presented on their surface, that include: administering a therapeutically effective amount of any of the pharmaceutical compositions described herein or any of the ABPCs described herein to a subject identified as having a cancer characterized by having the population of cancer cells. In some embodiments of any of the methods described herein, the volume of at least one tumor (e.g., solid tumor) or tumor location (e.g., a site of metastasis) is reduced (e.g., a detectable reduction) by ≥1%, ≥2%, ≥3%, ≥4%, ≥5%, ≥6%, ≥8%, ≥10%, ≥12%, ≥14%, ≥16%, ≥18%, ≥20%, ≥22%, ≥24%, ≥26%, ≥28%, ≥30%, ≥35%, ≥40%, ≥45%, ≥50%, ≥55%, ≥60%, ≥65%, ≥70%, ≥75%, ≥80%, ≥85%, ≥90%, ≥95%, or ≥99%) reduced as compared to the size of the at least one tumor (e.g., solid tumor) before administration of the ABPC.

Also provided herein are methods of inducing cell death in a cancer cell in a subject, wherein the cancer cell has TROP2 or an epitope of TROP2 presented on its surface, that include: administering a therapeutically effective amount of any of the pharmaceutical compositions of described herein or any of the ABPCs described herein to a subject identified as having a cancer characterized as having the population of cancer cells. In some embodiments, the cell death that is induced is necrosis. In some embodiments, the induced cell death is apoptosis.

In some embodiments of any of the methods described herein, the cancer is a primary tumor or a metastasis.

In some embodiments of any of the methods described herein, the cancer is a non-T-cell-infiltrating tumor. In some embodiments of any of the methods described herein, the cancer is a T-cell-infiltrating tumor.

Provided herein are methods of decreasing the risk of developing a metastasis or decreasing the risk of developing an additional metastasis in a subject having a cancer, wherein the cancer is characterized by having a population of cancer cells that have TROP2 or an epitope of TROP2 presented on their surface, that include: administering a therapeutically effective amount of any of the pharmaceutical compositions of described herein or any of the ABPCs described herein to a subject identified as having a cancer characterized as having the population of cancer cells. In some embodiments, the risk of developing a metastasis or the risk of developing an additional metastasis is decreased by ≥1%, by ≥2%, ≥3%, ≥4%, ≥5%, ≥6%, ≥8%, ≥10%, ≥12%, ≥14%, ≥16%, ≥18%, ≥20%, ≥25%, ≥30%, ≥35%, ≥40%, ≥45%, ≥50%, ≥55%, ≥60%, ≥65%, ≥70%, ≥75%, ≥80%, ≥85%, ≥90%, ≥95%, or ≥99% in the subject as compared to the risk of a subject having a similar cancer, but administered no treatment or a treatment that does not include the administration of any of the ABPCs described herein.

In some embodiments of any of the methods described herein, the cancer is a non-T-cell-infiltrating tumor. In some embodiments of any of the methods described herein, the cancer is a T-cell-infiltrating tumor. In some embodiments of any of the methods described herein, the cellular compartment is part of the endosomal/lysosomal pathway. In some embodiments of any of the methods described herein, the cellular compartment is an endosome.

The term “subject” refers to any mammal. In some embodiments, the subject or “subject suitable for treatment” may be a canine (e.g., a dog), feline (e.g., a cat), equine (e.g., a horse), ovine, bovine, porcine, caprine, primate, e.g., a simian (e.g., a monkey (e.g., marmoset, baboon), or an ape (e.g., a gorilla, chimpanzee, orangutan, or gibbon) or a human; or rodent (e.g., a mouse, a guinea pig, a hamster, or a rat). In some embodiments, the subject or “subject suitable for treatment” may be a non-human mammal, especially mammals that are conventionally used as models for demonstrating therapeutic efficacy in humans (e.g., murine, lapine, porcine, canine or primate animals) may be employed.

As used herein, treating includes reducing the number, frequency, or severity of one or more (e.g., two, three, four, or five) signs or symptoms of a cancer in a patient having a cancer (e.g., any of the cancers described herein). For example, treatment can reduce cancer progression, reduce the severity of a cancer, or reduce the risk of re-occurrence of a cancer in a subject having the cancer

Provided herein are methods of inhibiting the growth of a solid tumor in a subject (e.g., any of the subjects described herein) that include administering to the subject a therapeutically effective amount of any of the ABPCs described herein or any of the pharmaceutical compositions described herein (e.g., as compared to the growth of the solid tumor in the subject prior to treatment or the growth of a similar solid tumor in a different subject receiving a different treatment or receiving no treatment).

In some embodiments of any of the methods described herein, the growth of a solid tumor is primary growth of a solid tumor. In some embodiments of any of the methods described herein, the growth of a solid tumor is recurrent growth of a solid tumor. In some embodiments of any of the methods described herein, the growth of a solid tumor is metastatic growth of a solid tumor. In some embodiments, treatment results in about a 1% decrease to about 99% decrease (or any of the subranges of this range described herein) in the growth of a solid tumor in the subject (e.g., as compared to the growth of the solid tumor in the subject prior to treatment or the growth of a similar solid tumor in a different subject receiving a different treatment or receiving no treatment). The growth of a solid tumor in a subject can be assessed by a variety of different imaging methods, e.g., positron emission tomography, X-ray computed tomography, computed axial tomography, and magnetic resonance imaging.

Also provided herein are methods of decreasing the risk of developing a metastasis or developing an additional metastasis over a period of time in a subject identified as having a cancer (e.g., any of the exemplary cancers described herein) that include administering to the subject a therapeutically effective amount of any of the proteins described herein or any of the pharmaceutical compositions described herein (e.g., as compared to a subject having a similar cancer and receiving a different treatment or receiving no treatment). In some embodiments of any of the methods described herein, the metastasis or additional metastasis is one or more to a bone, lymph nodes, brain, lung, liver, skin, chest wall including bone, cartilage and soft tissue, abdominal cavity, contralateral breast, soft tissue, muscle, bone marrow, ovaries, adrenal glands, and pancreas.

In some embodiments of any of the methods described herein, the period of time is ~1 month to ~3 years (e.g., ~1-30, 1-24, 2-18, 1-12, 1-10, 1-8, 1-6, 1-5, 1-4, 1-3, 1-2, 2-36, 2-30, 2-24, 2-18, 2-12, 2-10, 2-8, 2-6, 2-5, 2-4, 2-3, 3-36, 3-30, 3-24, 3-18, 3-12, 3-10, 3-8, 3-6, 3-5, 3-4, 4-36, 4-30, 4-24, 4-18, 4-12, 4-10, 4-8, 4-6, 4-5, 5-36, 5-30, 5-24, 5-18, 5-12, 5-10, 5-8, 5-6, 6-36, 6-30, 6-24, 6-18, 6-12, 6-10, 6-8, 8-36, 8-30, 8-24, 8-18, 8-12, 8-10, 10-36, 10-30, 10-24, 10-18, 10-12, 12-36, 12-30, 12-24, 12-18, 18-36, 18-30, 18-24, 24-36, 24-30, or ~30-36 months).

In some embodiments, the risk of developing a metastasis or developing an additional metastasis over a period of time in a subject identified as having a cancer is decreased by ~1% to ~99% (e.g., or any of the subranges of this range described herein), e.g., as compared to the risk in a subject having a similar cancer receiving a different treatment or receiving no treatment.

Non-limiting examples of cancer include: acute lymphoblastic leukemia (ALL), acute myeloid leukemia (AML), adrenocortical carcinoma, anal cancer, appendix cancer, astrocytoma, basal cell carcinoma, brain tumor, bile duct cancer, bladder cancer, bone cancer, breast cancer, bronchial tumor, Burkitt Lymphoma, carcinoma of unknown primary origin, cardiac tumor, cervical cancer, chordoma, chronic lymphocytic leukemia (CLL), chronic myelogenous leukemia (CML), chronic myeloproliferative neoplasm, colon cancer, colorectal cancer, craniopharyngioma, cutaneous T-cell lymphoma, ductal carcinoma, embryonal tumor, endometrial cancer, ependymoma, esophageal cancer, esthesioneuroblastoma, fibrous histiocytoma, Ewing sarcoma, eye cancer, germ cell tumor, gallbladder cancer, gastric cancer, gastrointestinal carcinoid tumor, gastrointestinal stromal tumor, gestational trophoblastic disease, glioma, head and neck cancer, hairy cell leukemia, hepatocellular cancer, histiocytosis, Hodgkin lymphoma, hypopharyngeal cancer, intraocular melanoma, islet cell tumor, Kaposi sarcoma, kidney cancer, Langerhans cell histiocytosis, laryngeal cancer, leukemia, lip and oral cavity cancer, liver cancer, lobular carcinoma in situ, lung cancer, lymphoma, macroglobulinemia, malignant fibrous histiocytoma, melanoma, Merkel cell carcinoma, mesothelioma, metastatic squamous neck cancer with occult primary, midline tract carcinoma involving NUT gene, mouth cancer, multiple endocrine neoplasia syndrome, multiple myeloma, mycosis fungoides, myelodysplastic syndrome, myelodysplastic/myeloproliferative neoplasm, nasal cavity and para-nasal sinus cancer, nasopharyngeal cancer, neuroblastoma, non-Hodgkin lymphoma, non-small cell lung cancer, oropharyngeal cancer, osteosarcoma, ovarian cancer, pancreatic cancer, papillomatosis, paraganglioma, parathyroid cancer, penile cancer, pharyngeal cancer, pheochromocytomas, pituitary tumor, pleuropulmonary blastoma, primary central nervous system lymphoma, prostate cancer, rectal cancer, renal cell cancer, renal pelvis and ureter cancer, retinoblastoma, rhabdoid tumor, salivary gland cancer, Sezary syndrome, skin cancer, small cell lung cancer, small intestine cancer, soft tissue sarcoma, spinal cord tumor, stomach cancer, T-cell lymphoma, teratoid tumor, testicular cancer, throat cancer, thymoma and thymic carcinoma, thyroid cancer, urethral cancer, uterine cancer, vaginal cancer, vulvar cancer, and Wilms' tumor. Additional examples of cancer are known in the art.

In some embodiments, the patient is further administered one or more additional therapeutic agents (e.g., one or more of a chemotherapeutic agent, a recombinant cytokine or interleukin protein, a kinase inhibitor, and a checkpoint inhibitor). In some embodiments, the one or more additional therapeutic agents is administered to the patient at approximately the same time as any of the ABPCs described herein are administered to the patient. In some embodiments, the one or more additional therapeutic agents are administered to the patient after the administration of any of the ABPCs described herein to the patient. In some embodiments, the one or more additional therapeutic agents are administered to the patient before the administration of any of the ABPCs described herein to the patient.

In some embodiments of any of the methods described herein, the cancer is a solid cancer (e.g., breast cancer, prostate cancer, or non-small cell lung cancer).

### Compositions

Also provided herein are compositions (e.g., pharmaceutical compositions) that include at least one of any of the ABPCs described herein. In some embodiments, the compositions (e.g., pharmaceutical compositions) can be disposed in a sterile vial or a pre-loaded syringe.

In some embodiments, the compositions (e.g., pharmaceutical compositions) are formulated for different routes of administration (e.g., intravenous, subcutaneous, intramuscular, or intratumoral). In some embodiments, the compositions (e.g., pharmaceutical compositions) can include a pharmaceutically acceptable carrier (e.g., phosphate buffered saline). Single or multiple administrations of any of the pharmaceutical compositions described herein can be given to a subject depending on, for example: the dosage and frequency as required and tolerated by the patient. A dosage of the pharmaceutical composition should provide a sufficient quantity of the ABPC to effectively treat or ameliorate conditions, diseases, or symptoms.

Also provided herein are methods of treating a subject having a cancer (e.g., any of the cancers described herein) that include administering a therapeutically effective amount of at least one of any of the compositions or pharmaceutical compositions provided herein.

### Kits

Also provided herein are kits that include any of the ABPCs described herein, any of the compositions described herein, or any of the pharmaceutical compositions described herein In some embodiments, the kits can include instructions for performing any of the methods described herein. In some embodiments, the kits can include at least one dose of any of the compositions (e.g., pharmaceutical compositions) described herein. In some embodiments, the kits can provide a syringe for administering any of the pharmaceutical compositions described herein.

### Protein Constructs

Also provided are protein constructs (PCs) that include: a first ABD that is capable of specifically binding TROP2 or an epitope of TROP2 presented on the surface of a target mammalian cell, where: (a) the dissociation rate of the first ABD at a pH of about 7.0 to about 8.0 (or any of the subranges of this range described herein) is faster than the dissociation rate at a pH of about 4.0 to about 6.5 (or any of the subranges of this range described herein); and/or (b) the dissociation constant (K_{D}) of the first ABD at a pH of about 7.0 to about 8.0 (or any of the subranges of this range) is greater than the K_{D} at a pH of about 4.0 to about 6.5.

Also provided herein are pharmaceutical compositions including any of the PCs described herein. Also provided herein are methods of treating a subject in need thereof that include administering a therapeutically effective amount of any of the PCs described herein to the subject.

### Methods of Improving pH Dependence of an Antigen-Binding Protein Construct

Also provided herein are methods of improving pH dependence of an antigen-binding protein construct, the method comprises providing a starting antigen-binding protein construct comprising an ABD and introducing one or more histidine amino acid substitutions into one or more CDRs of the ABD in the starting antigen-binding protein construct, wherein the method results in the generation of an antigen-binding protein construct having one or both of: (a) an increased (e.g., at least a 0.1-fold increase to about a 100-fold increase, or any of the subranges of this range described herein) ratio of the dissociation rate of the ABD at a pH of about 4.0 to about 6.5 to the dissociation rate at a pH of about 7.0 to about 8.0, as compared to the starting antigen-binding protein construct, and (b) an increased (e.g., at least a 0.1-fold increase to about a 100-fold increase, or any of the subranges of this range described herein) ratio the dissociation constant (K_{D}) of the ABD at a pH of about 4.0 to about 6.5 to the K_{D} at a pH of about 7.0 to about 8.0, as compared to the starting antigen-binding protein construct.

From the foregoing, it will be seen that aspects herein are well adapted to attain all the ends and objects hereinabove set forth together with other advantages which are obvious and which are inherent to the structure.

While specific elements and steps are discussed in connection to one another, it is understood that any element and/or steps provided herein is contemplated as being combinable with any other elements and/or steps regardless of explicit provision of the same while still being within the scope provided herein.

It will be understood that certain features and subcombinations are of utility and may be employed without reference to other features and subcombinations. This is contemplated by and is within the scope of the claims.

Since many possible aspects may be made without departing from the scope thereof, it is to be understood that all matter herein set forth or shown in the accompanying drawings and detailed description is to be interpreted as illustrative and not in a limiting sense.

It is also to be understood that the terminology used herein is for the purpose of describing particular aspects only, and is not intended to be limiting. The skilled artisan will recognize many variants and adaptations of the aspects described herein. These variants and adaptations are intended to be included in the teachings of this disclosure and to be encompassed by the claims herein.

Now having described the aspects of the present disclosure, in general, the following Examples describe some additional aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of the present disclosure.

### EXAMPLES

### Example 1. Generation of TROP2 Binders and Engineering of pH Binding Dependence

pH-engineered ABPCs specific for TROP2 are generated using two methods. In the first approach, published monoclonal antibodies against TROP2 are used as a starting template for introduction of additional mutations that allow engineering of pH-dependent binding to TROP2 and i) enhanced endolysosomal accumulation of a conjugated toxin, as well as ii) enhanced TROP2 recycling to the cell surface. The second approach involves discovery of de novo ABPCs specific for TROP2 via antibody display methods from naive libraries or libraries with defined CDR compositions and screening under conditions designed for selection of pH-engineered ABPCs specific for TROP2. In either case, histidine residues play an important role in engineering pH-dependent binding proteins.

Histidine residues are at least partially protonated at a pH below 6.5 owing to its pKa of 6.0. Therefore, if a histidine side chain in an ABD participates in an electrostatic binding interaction with its antigen it will start to turn positively charged at a pH at or below 6.5. This could either weaken or enhance the binding affinity of the interaction at a pH below 6.5, based on the corresponding charge of and interactions with the antigen epitope. Thus, systematic introduction of histidines into antibody complementarity determining regions (CDRs) in an antibody or other binder library (e.g., an scFv library) can be used to identify substitutions that will affect an ABD's interaction with an antigen at lower pH values. The first approach therefore involves histidine-scanning of variable region sequences of published monoclonal antibodies to identify pH-dependent variants.

Multiple TROP2-binding monoclonal antibodies have been described in the literature and can be used as a template for engineering pH-dependent binding. Briefly, for a subset of the antibody sequences, CDRs in each chain are identified using the methods described by Kabat et al (Kabat et al. (1992) Sequences of Proteins of Immunological Interest (DIANE publishing) and IMGT (Lefranc M P (1999) “The IMGT unique numbering for Immunoglobulins, T cell receptors and Ig-like domains” The Immunologist 7, 132-136), and for each CDR, residues falling under either or both Kabat and IMGT CDR definitions were called as CDR residues. To engineer pH-dependent sequence variants, individual amino acid residues within the heavy chain and/or light chain CDRs are systematically substituted with a histidine, one at a time. In cases where the starting CDR residue is a histidine, it is mutated to an alanine. Antibody variants with only one histidine or alanine mutation in a heavy/light chain CDR are generated by co-transfection of Expi293 cells with a) one heavy chain or light chain sequence variant, and b) the corresponding starting ABPC (e.g., the starting TROP2-binding monoclonal antibody) light chain or heavy chain, respectively, using methods known to the art. After allowing for a period of protein expression, cell culture supernatants are collected, quantified, and the pH dependence of the variant is evaluated using biolayer interferometry (BLI) or other methods known to the art. Briefly, cell culture supernatants are normalized to an antibody expression level of 50 μg/mL, and captured on an anti-human Fc sensor (Forte Bio). A baseline is established using 1× kinetics buffer (Forte Bio), and the sensor is associated with 100 nM of TROP2 in 1×PBS at pH 7.4 for 300 sec to generate an association curve. In the dissociation phase, the antibody-antigen complex on the sensor is exposed to 1×PBS at either pH 5.5 or pH 7.4 for 300-500 sec. Association and dissociation phase curves are examined for the starting ABPC antibody and each corresponding antibody variant at pH 5.5 and pH 7.4 to inform on two criteria: a) enhanced dissociation (i.e., higher koff values) at pH 5.5 due to histidine or alanine substitution compared to the starting ABPC, and b) reduced dissociation at pH 7.4 (i.e., lower koff values) compared to pH 5.5 in the antibody variant itself and with the starting ABPC. Variants that show either enhanced dissociation at pH 5.5 or reduced dissociation at pH 7.4 or both are selected for further analysis. It is also noted that while some histidine and alanine mutations obliterate TROP2 binding, others are tolerated with little (e.g., less than 1-fold change in KD or dissociation rate) or no change in TROP2 binding kinetics. Especially because histidine is a large, positively charged amino acid, these histidine variants and alanine variants with no change are noted as positions that may tolerate a wide range of mutations and lead to antibodies with different sequence but similar binding properties, a designation that is not otherwise apparent. The variants selected for further analysis are expressed at a larger scale and purified using protein A affinity chromatography. Binding kinetics (kon and koff) of the purified starting ABPC and variant antibodies are measured at pH 5.5 and pH 7.4 using Biacore (GE Healthcare). The ratio of the antibody's rate of dissociation (koff at pH 7.4 divided by koff at pH 5.5) is also used as a quantitative assessment of pH-dependent binding; similarly, the dissociation constant KD is calculated at both pH 5.5 and pH 7.4 as koff divided by kon and the ratio of the antibody's dissociation constant (KD at pH 7.4 divided by KD at pH 5.5) is also used as a quantitative assessment of pH-dependent binding. Antibodies with a rate of dissociation ratio less than that of the starting ABPC and/or a dissociation constant ratio less than that of the starting ABPC are selected for further assessment of combinatorial substitutions. Favorable histidine and/or alanine amino acid positions can also be combined to enhance pH dependence; this can be done by, e.g., combinatorially or rationally combining histidine and/or alanine substitutions on a given heavy or light chain that individually improve pH dependence, by, e.g., combinatorially or rationally combining modified heavy and light chains such that histidine and/or alanine substitutions are present on both chains, or combinations thereof. Such combinatorial variants are generated and tested/analyzed for differential pH dependence using the methods and protocols described herein, or others known to the art. Antibody variants that have the lowest rate of dissociation ratios and/or dissociation constant ratios are selected as candidates for further analysis (hereafter referred to as “pH-engineered ABPCs specific for TROP2”).

The second method for selection of pH-engineered ABPCs specific for TROP2 involves either screening libraries to identify de novo pH-dependent ABPCs specific for TROP2 or ABPCs that could serve as templates for engineering pH-dependent binding as described herein. Two types of libraries can be used for these selections: naive phage/yeast display antibody libraries (e.g., Fab, scFv, VHH, VL, or others known to the art) or phage/yeast display libraries where CDRs have been mutated to express a subset of amino acid residues. Libraries are screened against soluble recombinant TROP2 extracellular domains using methods known to the art with positive selection for variants that bind weakly (e.g., are eluted from beads) at pH 5.0 and bind strongly (e.g., are bound to beads) at pH 7.4. Three rounds of selections are performed. The final round of binders are screened using ELISA for binding to human TROP2 and cyno TROP2 and mouse TROP2 or via mean fluorescence intensity (MFI) in flow cytometric analysis. If more binders with cyno or murine cross-reactivity are desired, the final selection round can instead be performed on cyno TROP2 or murine TROP2. Selected binding proteins are subcloned into mammalian expression vectors and expressed as either full IgG proteins or Fc fusions in Expi293 cells. BLI analysis is performed as described herein for selection of pH-dependent binder variants and confirmed using Biacore.

### Example 2. In Vitro Demonstration of pH-Dependent Binding to TROP2, pH-Dependent Release of TROP2, Enhanced Endolysosomal Delivery in TROP2+ Cells, and Increased TROP2 Antigen Density in TROP2+ Cells after Exposure to pH-Engineered ABPCs Specific For TROP2 as Compared to Control ABPCs Specific for TROP2

As discussed herein, pH-engineered ABPCs specific for TROP2 exhibit the desirable property of decreased TROP2 binding at acidic pH (e.g., pH 5.0 or pH 5.5), but enhanced binding at higher pH (e.g., pH 7.4), which enhances their accumulation in endolysosomes under physiological conditions.

pH-Dependent Binding to TROP2 on Cells

To demonstrate that pH-engineered ABPCs specific for TROP2 binds cell surface TROP2 at neutral pH, a cell surface binding assay is performed. A panel of human cells that are TROP2+ is assembled (e.g., NCI-H1975, MKN-45, DSMZ ACC 409; HPAC, ATCC CRL-2119; HPAF-IL, ATCC CRL-1997). Methods of identifying and quantifying gene expression (e.g., TROP2) for a given cell line are known to the art, and include, e.g., consulting the Cancer Cell Line Encyclopedia to ascertain the expression level and/or mutation status of a given gene in a tumor cell line), rtPCR, microarray, or RNA-Seq analysis, or cell staining with anti-TROP2 antibodies known in the art (e.g., sacituzumab, datopotamab, hu7E6SVG, or A1,2X4). Cells are seeded at approximately 5-10,000 per well in 150 μL of pH 7.4 culture medium and incubated at 37° C. for 5 minutes at several doses (e.g., a two-fold dilution series) from 1 pM to 1 μM with one of the following antibodies: a known, control ABPC specific for TROP2 (e.g., an antibody, sacituzumab, datopotamab, hu7E6SVG, or A1,2X4), the pH-engineered ABPC specific for TROP2, and an appropriate negative isotype control mAb (e.g., Biolegend Purified Human IgG1 Isotype Control Recombinant Antibody, Cat #403501). Prior to the onset of the experiment, the binding properties of all antibodies are validated using methods known to the art. Following the 5 minute incubation, cells are fixed with 4% formaldehyde (20 min at room temperature) and incubated with an appropriate fluorophore-labeled secondary antibody (e.g., ThermoFisher Mouse anti-Human IgG1 Fc Secondary Antibody, Alexa Fluor 488, Cat #A-10631) for 60 minutes. Unbound reagents are washed with a series of PBS washes, and the cell panels are imaged using confocal microscopy. Upon analysis of the images, significant fluorescence can be observed on the surface of cells bound with the known, control ABPC specific for TROP2 as well as the pH-engineered ABPC specific for TROP2, but little surface binding can be observed for the isotype negative control. To isolate the effect of pH on surface binding, the same experiment is repeated twice, with the primary antibody incubation taking place at sequentially lower pH (e.g., pH 6.5 and 5.5 and 5.0). Analysis of the resulting confocal microscopy images can show significant fluorescence on the surface of cells bound with all mAbs tested, excepting the isotype negative control, and that this fluorescence decreases for the pH-engineered ABPC specific for TROP2 as the pH decreases. Alternatively, cells are analyzed for mean fluorescent intensity by flow cytometry using methods known in the art. A dissociation constant KD on cells at neutral pH of the antibodies analyzed is determined by nonlinear regression methods known in the art (e.g., a Scatchard plot). Taken together, the results can show that the pH engineering process results in the creation of a pH-engineered ABPC specific for TROP2 that is pH-dependent in its binding properties and that it more effectively binds at neutral pH as compared to more acidic pH. Other methods of assessing the pH dependence of the pH-engineered ABPCs specific for TROP2 are known in the art and include, e.g., using flow cytometry to measure ABPC surface binding.

pH-Dependent Release of TROP2 on Cells

To demonstrate that pH-engineered ABPCs specific for TROP2 are capable of releasing TROP2 at low pH after binding at a neutral pH, a variant of the cell surface binding assay described above is performed using methods known to the art (e.g., as generally described in Gera N. (2012) PLoS ONE 7(11): e48928). Briefly, an appropriate TROP2+ cell line is harvested and 50,000 cells per well are plated in a U-Bottomed 96-well microplate. Three conditions are tested; binding and secondary staining at pH 7.4, binding and secondary staining at pH 5.0, and binding at pH 7.4 followed by release at pH 5.0 for 30 minutes and secondary staining at pH 7.4. Both pH-engineered ABPCs specific for TROP2 as well as a control ABPC specific for TROP2 are tested. The cells are washed two times with 200 μL of FACS buffer (1×PBS containing 3% Fetal Bovine Serum) at either pH 7.4 or 5.0 depending on the condition being tested. The purified protein samples are diluted into FACS buffer of the appropriate pH and added to the cells and allowed to bind for one hour on ice. After incubation with the primary antibodies the pH 7.4 and pH 5.0 conditions are washed twice as before, and then 100 μl of secondary rat anti-human Fc AF488 (BioLegend 410706) or other appropriate antibody, diluted 1:50, or anti Myc-Tag mouse mAb-AF488 (Cell Signaling Technologies 2279S) diluted 1:50 is added in FACS buffer of the appropriate pH, and incubated for 30 minutes on ice. The pH 5.0 release condition is washed twice with FACS buffer pH 7.4 and then resuspended in 100 μl of FACS buffer pH 5.0 and incubated on ice for 30 minutes, followed by secondary staining in FACS buffer pH 7.4 as described for the other conditions. The plates are washed twice as before and resuspended in 1% paraformaldehyde in the appropriate FACS buffer to fix them for flow cytometry analysis. All conditions are read on a flow cytometer (Accuri C6, BD Biosciences). Binding is observed as a shift in the FLI signal (as a MFI) versus secondary alone. Upon analysis of the data, it can be determined that both the pH-engineered ABPC specific for TROP2 as well as the control ABPC specific for TROP2 effectively bind the surface of TROP2+ cells at neutral pH, but the pH-engineered ABPC specific for TROP2 binds poorly at pH 5.0; similarly, it can be determined that the pH-engineered ABPC specific for TROP2 binds effectively at pH 7.4, but then releases/unbinds TROP2 at pH 5.0.

Enhanced Endolysosomal Delivery in TROP2+ Cells of pH-Engineered ABPCs Specific for TROP2 as Compared to Control ABPCs Specific for TROP2 (pHrodo)

To verify and demonstrate that ABPCs specific for TROP2 achieve endolysosomal localization following cellular uptake, an internalization assay is performed using methods known to the art (e.g., Mahmutefendic et al., Int. J. Biochem. Cell Bio., 2011). Briefly, as described herein, a panel of human cells that express TROP2 highly is assembled using methods known to the art. Cells are plated, washed three times with PBS, and incubated at 37° C. for 60 minutes in media at neutral pH, with added concentrations of 2 micrograms per milliliter of a known, control ABPC specific for TROP2 (e.g., as described herein), the pH-engineered ABPC specific for TROP2, and an appropriate negative isotype control mAb (e.g., as described herein). In a subset of cells, validation of antibody internalization and endosomal localization is performed using methods known to the art; e.g., cells are fixed in 4% formaldehyde as described herein, permeabilized using TWEEN 20 or other methods known to the art (Jamur M C et al (2010) Permeabilization of cell membranes, Methods Mol Biol. 588:63-6), additionally stained with an endosomal marker, e.g., a fluorescent RAB11 antibody (RAB11 Antibody, Alexa Fluor 488, 3H18L5, ABfinity™ Rabbit Monoclonal), stained with an appropriate fluorescently labeled anti-human secondary antibody (e.g., as described herein), and imaged using confocal fluorescence microscopy, as described herein. Analysis of the confocal images can be used to show that both the pH-engineered ABPC specific for TROP2 as well as the control ABPC specific for TROP2 are internalized and accumulate in the endolysosomes.

To demonstrate that pH-engineered ABPCs specific for TROP2 achieve enhanced endolysosomal accumulation relative to a control ABPC specific for TROP2, a pHrodo-based internalization assay is performed using both a known, control ABPC specific for TROP2 as well as the pH-engineered ABPC specific for TROP2. The assay makes use of pHrodo™ iFL (P36014, ThermoFisher), a dye whose fluorescence increases with decreasing pH, such that its level of fluorescence outside the cell at neutral pH is lower than its level of fluorescence inside the acidic pH environment of endolysosomes. Briefly, an appropriate TROP2+ cell line is suspended in its recommended media and plated in a 24-well plate at a density of 2,000,000 cells/mL, 1 mL per well. While keeping the cells on ice, 1 mL of 2×pHrodo iFL-labeled antibody is added to each well, the well is pipetted/mixed five times, and the plate is incubated in a light-protected environment for 45 minutes, on ice. An identical but separate plate is also incubated on ice that is meant as a no-internalization negative control. Following this incubation, the experimental plate is moved to a 37° C. incubator, the negative control plate is kept on ice to slow or block internalization, and samples are taken at designated time points to create an internalization time course. Samples are placed into a U-bottom 96-well plate, and internalization is quenched via addition of 200 μL/well of ice-cold FACS buffer. The plates are spun down at 2000×g for 2 minutes, resuspended in 200 μL ice-cold FACS buffer, spun down again, and resuspended in FACS buffer a second time. Finally, the samples are loaded into a flow cytometer for read-out of cellular pHrodo fluorescence using excitation and emission wavelengths consistent with the excitation and emission maxima of the pHrodo iFL Red dye (566 nm and 590 nm, respectively). Upon completion of the flow cytometry experiment and analysis of the data, it can be observed that cells treated with the pH-engineered ABPC specific for TROP2 have a higher pHrodo iFL signal relative to a known, control ABPC specific for TROP2, indicating that pH-engineered ABPCs specific for TROP2 achieve enhanced endolysosomal accumulation relative to a control ABPC specific for TROP2.

Alternatively, to demonstrate that pH-engineered ABPCs specific for TROP2 achieve enhanced endolysosomal accumulation relative to a control ABPC specific for TROP2, a variation of the above-described experiment is performed. TROP2+ cells are plated, washed three times with PBS, and incubated at 37° C. for 60 minutes in media at neutral pH with added concentrations of 2 μg/mL of either pH-engineered ABPC specific for TROP2 or control ABPC specific for TROP2. Following incubation, cells are washed three times with PBS, fixed and permeabilized, and stained with a panel of appropriately selected antibodies that bind late endosomal markers as well as lysosomes (e.g., RAB7, and LAMP1; Cell Signaling Technology, Endosomal Marker Antibody Sampler Kit #12666; AbCam, Anti-LAMP2 antibody [GL2A7], ab13524). After primary antibody staining, cells are stained with an appropriate mixture of fluorescently labeled secondary antibodies (e.g., Goat Anti-Human lgG (H&L) Secondary Antibody (Alexa Fluor 647) Cat #A-21445, and Abcam Goat Anti-Rabbit IgG H&L (Alexa Fluor 488), Cat #ab150077), imaged using confocal fluorescence microscopy, and regions of co-localization of signal from TROP2-specific antibodies and endosomal markers are visualized and quantified. Upon analysis of the data, it can be revealed that there is increased co-localization of endolysosomal and TROP2-specific antibody signal in wells treated with the pH-engineered ABPCs specific for TROP2 as compared to wells treated with control ABPC specific for TROP2, and can thereby demonstrate that pH-engineered ABPCs specific for TROP2 achieve enhanced endolysosomal accumulation relative to control ABPC specific for TROP2.

Increased TROP2 Antigen Density in TROP2+ Cells after Exposure to pH-Engineered ABPCs Specific for TROP2 as Compared to Control ABPCs Specific for TROP2

To demonstrate that treatment of cells with the pH-engineered ABPCs specific for TROP2 does not result in a detectable reduction of the level of TROP2 on the surface of cells exposed to the pH-engineered ABPCs specific for TROP2, or that said treatment results in less of a reduction of the level of TROP2 on the surface of cells exposed to the pH-engineered ABPC specific for TROP2 versus a control ABPC specific for TROP2, an antigen density study is performed using flow cytometry. Briefly, 4.0×10\hat{ }5 cells that express TROP2 are plated per well in a 96-well plate in 100 μL media. Cells are treated with a titration from 1 pM to 1 μM of i) pH-engineered ABPCs specific for TROP2, ii) a first control ABPC specific for TROP2, iii) an appropriate isotype control, and iv) an untreated control. Cells are incubated for 2 hours at 37° C., at which point all cells are incubated with 200 nM of a fluorophore-labeled second control ABPC specific for TROP2 (e.g., as described herein) which has a different epitope (as determined by, e.g., competitive binding studies on cells) than either the first control ABPC specific for TROP2 or the pH-engineered ABPCs specific for TROP2 for 30 minutes at 4° C. Following this 30-minute incubation, the MFI (MFI) of all cells is read out using, e.g., flow cytometry, using methods known to one of ordinary skill in the art. In parallel, a quantitative standard curve that can be used to quantify the presence of TROP2 on the surface of treated cells as a function of MFI is generated using a commercially available quantification kit (e.g., BD Biosciences PE Phycoerythrin Fluorescence Quantitation Kit, catalog #340495); the quantitative standard curve is created by following the manufacturer's instructions. Other methods of determining the absolute number of TROP2 on the cell surface are known in the art and include, e.g., use of radioisotopically labeled reagents. Upon analysis of the data, it can be revealed that at least one antibody concentration, cells treated with a control ABPC specific for TROP2 experience a reduction of the level of TROP2 on their surface, whereas cells treated with pH-engineered ABPCs specific for TROP2 experience a significantly smaller reduction or no reduction at all, both relative to the isotype and untreated controls.

### Example 3. Conjugation of pH-Engineered and Control ABPCs Specific for TROP2 to Cytotoxic Drugs

An antigen-binding protein construct conjugate (ADC) is made comprising the TROP2-binding IgG (hereafter, TROP2-IgG) described herein linked to monomethyl auristatin E (MMAE) via a valine-citrulline (vc) linker (hereafter, TROP2-IgG-DC). Conjugation of the antigen-binding protein construct with vcMMAE begins with a partial reduction of the TROP2-IgG followed by reaction with maleimidocaproyl-Val-Cit-PABC-MMAE (vcMMAE). The TROP2-IgG (20 mg/mL) is partially reduced by addition of TCEP (molar equivalents of TCEP:mAb is 2:1) followed by incubation at 0° C. overnight. The reduction reaction is then warmed to 20° C. To conjugate all of the thiols, vcMMAE is added to a final vcMMAE:reduced Cys molar ratio of 1:15. The conjugation reaction is carried out in the presence of 10% v/v of DMSO and allowed to proceed at 20° C. for 60 minutes.

After the conjugation reaction, excess free N(acetyl)-Cysteine (2 equivalents vs. vcMMAE charge) is added to quench unreacted vcMMAE to produce the Cys-Val-Cit-MMAE adduct. The Cys quenching reaction is allowed to proceed at 20° C. for approximately 30 minutes. The Cys-quenched reaction mixture is purified as per below. The above conjugation method can also be used to conjugate maleimidocaproyl monomethylauristatin F (mcMMAF) to an antigen-binding protein construct.

The TROP2-IgG-DC is purified using a batch purification method. The reaction mixture is treated with the appropriate amount of water washed Bu-HIC resin (ToyoPearl; Tosoh Biosciences), i.e., seven weights of resin is added to the mixture. The resin/reaction mixture is stirred for the appropriate time, and monitored by analytical hydrophobic interaction chromatography for removal of drug conjugate products, filtered through a coarse polypropylene filter, and washed by two bed volumes of a buffer (0.28 M sodium chloride, 7 mM potassium phosphate, pH 7). The combined filtrate and rinses are combined and analyzed for product profile by HIC HPLC. The combined filtrate and rinses are buffer exchanged by ultrafiltration/diafiltration (UF/DF) to 15 mM histidine, pH 6 with 10 diavolumes 15 nM histidine buffer.

A similar protocol can be used to conjugate DNA toxins such as SG3249 and SGD-1910 to TROP2-IgG (see Tiberghien A C et al (2016) Design and Synthesis of Tesirine, a Clinical Antibody-Drug Conjugate Pyrrolobenzodiazepine Dimer Payload, ACS Med Chem Lett 7:983-987). Briefly, for SG3249, TROP2-IgG (15 mg, 100 nanomoles) is diluted into 13.5 mL of a reduction buffer containing 10 mM sodium borate pH 8.4, 2.5 mM EDTA and a final antibody concentration of 1.11 mg/mL. A 10 mM solution of TCEP is added (1.5 molar equivalent/antibody, 150 nanomoles, 15 microliters) and the reduction mixture is heated at +37° C. for 1.5 hours in an incubator. After cooling down to room temperature, SG3249 is added as a DMSO solution (5 molar equivalent/antibody, 500 nanomoles, in 1.5 mL DMSO). The solution is mixed for 1.25 hours at room temperature, then the conjugation is quenched by addition of N-acetyl cysteine (1 micromole, 100 microliters at 10 mM), and injected into an AKTA™ Pure FPLC using a GE Healthcare HiLoad™ 26/600 column packed with Superdex 200 PG, and eluted with 2.6 mL/min of sterile-filtered phosphate-buffered saline (PBS). Fractions corresponding to the TROP2-IgG-DC monomer peak are pooled, concentrated using a 15 mL Amicon Ultracell 50 KDa MWCO spin filter, analyzed and sterile-filtered. UHPLC analysis on a Shimadzu Prominence system using a Phenomenex Aeris 3.6 u XB-C18 150×2.1 mm column eluting with a gradient of water and acetonitrile on a reduced sample of TROP2-IgG-DC at 280 nm and 330 nm (SG3249 specific) can show a mixture of light and heavy chains attached to several molecules of SG3249, consistent with a drug-per-antibody ratio (DAR) of 1 to 4 molecules of SG3249 per antibody. UHPLC analysis on a Shimadzu Prominence system using a Phenomenex Yarra 3 u SEC-3000 300 mm×4.60 mm column eluting with sterile-filtered SEC buffer containing 200 mM potassium phosphate pH 6.95, 250 mM potassium chloride and 10% isopropanol (v/v) on a sample of TROP2-IgG-DC at 280 nm can show a monomer purity of over 90% with no impurity detected. UHPLC SEC analysis allows determination of final TROP2-IgG-DC yield of greater than 30%.

Alternatively, methods to conjugate toxins to antibodies via lysine residues are known in the art (e.g., see Catcott K C et al (2016) Microscale screening of antibody libraries as maytansinoid antibody-drug conjugates, MAbs 8:513-23). In addition, similar methods to the above can be used to conjugate drugs and toxins to non-IgG formats with disulfide bonds, such as Vh-Fcs.

### Example 4. Demonstration of Enhanced Cytotoxicity of pH-Engineered ABPC ADCs Specific For TROP2 in TROP2+ Cells as Compared to a Control ABPC ADC Specific for TROP2

The cytotoxic activity of both pH-engineered ADCs specific for TROP2 (e.g., a pH-engineered TROP2-IgG-DC) and control ABPC ADCs specific for TROP2 (e.g., a control ABPC TROP2-IgG-DC) are separately evaluated on a panel of TROP2+ cell lines expressing a variety of antigen densities (e.g., as described herein) and a TROP2− cell line, selected using the methods described herein, and, optionally, cells expressing transgenic TROP2, e.g., HEK293 cells transfected with TROP2 using methods known in the art (e.g., Expi293™ Expression System Kit ThermoFisher Catalog number: A14635). For purposes of validation, prior to use, all cell lines are tested for expression of TROP2 using methods known to the art, e.g., qPCR, flow cytometry, mRNA RPKM, and antibody staining using anti-TROP2 antibodies known to the art (e.g., as described herein) followed by visualization of the stain using fluorescence microscopy, immunohistochemistry, flow cytometry, ELISA, or other methods known to the art. To evaluate the cytotoxicity of compounds, cells are seeded at approximately 10-40,000 per well in 150 microliters of culture medium, then treated with graded doses of compounds from 1 pM to 1 μM in quadruplicates at the initiation of the assay. Cytotoxicity assays are carried out for 96 hours after addition of test compounds. Fifty microliters of resazurin dye are added to each well during the last 4 to 6 hours of the incubation to assess viable cells at the end of culture. Dye reduction is determined by fluorescence spectrometry using the excitation and emission wavelengths of 535 nm and 590 nm, respectively. For analysis, the extent of resazurin reduction by the treated cells is compared to that of untreated control cells, and percent cytotoxicity is determined. Alternatively, a WST-8 kit is used to measure cytotoxicity per the manufacturer's instructions (e.g., Dojindo Molecular Technologies Catalog #CCK-8). IC50, the concentration at which half-maximal killing is observed, is calculated using curve-fitting methods known in the art. Upon analysis of the data, it can be determined that pH-engineered and control ABPC ADCs specific for TROP2 are substantially cytotoxic to one or more TROP2+ cell line, but less toxic to TROP2− cells. It also can be determined that pH-engineered ADCs specific for TROP2 are more cytotoxic to one or more TROP2+ cell lines than control ABPC ADCs specific for TROP2 because: a) they show greater depth of killing at one or more concentrations or, b) they show lower IC50 or, c) they show a greater ratio of their dissociation constant KD on cells at neutral pH (as described herein) divided by their IC50 on those same cells.

Additionally, the cytotoxic activity of ABPCs specific for TROP2 can be measured in a secondary ADC assay. Secondary ADC assays are known in the art (e.g., Moradec Cat #αHFc-NC-MMAF and Cat #αHFc-CL-MMAE, and associated manufacturer's instructions). Briefly, the assay is carried out as in the previous paragraph, except the ABPC specific for TROP2 is substituted for the ADC specific for TROP2, and to evaluate the cytotoxicity of compounds, cells are seeded at approximately 10-40,000 per well in 150 microliters of culture medium, then treated with graded doses of ABPC specific for TROP2 from 1 μM to 1 μM (final concentration in culture medium, having been pre-mixed with 100 nM, final concentration in culture medium, of Moradec Cat #αHFc-NC-MMAF secondary ADC reagent and pre-incubated at 37° C. for 30 min before addition of the mixture to the culture medium) in quadruplicates at the initiation of the assay.

The cytotoxic activity of pH-engineered ADCs specific for TROP2 and control ABPC ADCs specific for TROP2 conjugates, as well as ABPCs specific for TROP2 in a secondary ADC assay, are additionally measured by a cell proliferation assay employing the following protocol (Promega Corp. Technical Bulletin TB288; Mendoza et al., Cancer Res. 62:5485-5488, 2002):
1. An aliquot of 100 microliters of cell culture containing about 104 cells (e.g., TROP2+ cells as described herein) in medium is deposited in each well of a 96-well, opaque-walled plate.
2. Control wells are prepared containing medium and without cells.
3. ADC specific for TROP2 is added to the experimental wells at a range of concentrations from 1 pM-1 uM and incubated for 1-5 days. Alternatively, in a secondary ADC assay, 100 nM secondary ADC reagent (final concentration in culture medium, Moradec Cat #αHFc-NC-MMAF) and ABPC specific for TROP2 at a range of concentrations from 1 pM-1 uM (final concentration in culture medium) are pre-mixed and pre-incubated at 37° C. for 30 min before addition of the mixture to the culture medium, and incubated for 1-5 days.
4. The plates are equilibrated to RT for approximately 30 minutes.
5. A volume of CellTiter-Glo Reagent equal to the volume of cell culture medium present in each well is added.
6. The contents are mixed for 2 minutes on an orbital shaker to induce cell lysis.
7. The plate is incubated at RT for 10 minutes to stabilize the luminescence signal.
8. Luminescence is recorded; reported in graphs as RLU=relative luminescence units.

### Example 5. Demonstration of Enhanced Toxin Liberation of pH-Engineered ABPC ADCs Specific for TROP2 in TROP2+ Cells as Compared to a Control ABPC ADC Specific for TROP2

The pH-engineered ADCs specific for TROP2 (e.g., a pH-engineered TROP2-IgG-DC) can also demonstrate increased toxin liberation in TROP2+ cells as compared to a control ABPC ADC specific for TROP2 (e.g., a control ABPC TROP2-IgG-DC). After treatment of TROP2+ cells with pH-engineered and control ABPC ADCs specific for TROP2 as described herein, an LC-MS/MS method is used to quantify unconjugated (i.e., liberated) MMAE in treated TROP2+ cells (Singh, A. P. and Shah, D.K. Drug Metabolism and Disposition 45.11 (2017): 1120-1132.) An LC-MS/MS system with electrospray interphase and triple quadrupole mass spectrometer is used. For the detection of MMAE, a XBridge BEH Amide column (Waters, Milford, MA) is used with mobile phase A as water (with 5 mM ammonium formate and 0.1% formic acid) and mobile phase B as 95:5 acetonitrile/water (with 0.1% formic acid and 1 mM ammonium formate), using a gradient at a flow rate of 0.25 mL/min at 40° C. The total duration of the chromatographic run is 12 minutes, where two MRM scans (718.5/686.5 and 718.5/152.1 amu) are monitored. Deuterated (d8) MMAE (MCE MedChem Express, Monmouth Junction, NJ) is used as an internal standard. First, an equation for quantifying unconjugated MMAE in a biological sample is derived by dividing the peak area for each drug standard by the peak area obtained for the internal standard. The resultant peak area ratios are then plotted as a function of the standard concentrations, and data points are fitted to the curve using linear regression. Three QC samples are included in the low, middle, and upper ranges of the standard curve to assess the predictive capability of the developed standard curve. The standard curves obtained are then used to deduce the observed concentrations of MMAE in a biologic sample. For measurement of MMAE concentration, treated cell samples are pelleted and reconstituted in fresh media to a final concentration of 0.25 million cells/100 μL. Samples are spiked with d8-MMAE (1 ng/mL) before performing cell lysis by the addition of a 2-fold volume of ice-cold methanol followed by freeze-thaw cycle of 45 minutes at −20° C. The final cell lysate is obtained by centrifuging the samples at 13,000 rpm for 15 minutes at 4° C. followed by collection of supernatant. For the preparation of standards and QC samples, a fresh cell suspension (0.25 million/100 μl) is spiked with known concentrations of MMAE and internal standard (d8-MMAE) before a procedure similar to the cell lysis mentioned above. The resulting cell lysates are then evaporated and reconstituted in mobile phase B before injection into LC-MS/MS. The concentration of unconjugated MMAE in lysates of TROP2+ cells treated with pH-engineered ADCs specific for TROP2 is observed to be greater than that in TROP2+ cells treated with control ABPC ADC specific for TROP2.

For tubulin-inhibiting toxins, toxin liberation is also assessed by monitoring of cell viability and cell cycle phase. ~2.0×10\hat{ }5 TROP2+ cells are plated in a 96-well flat bottom plate and treated with pH-engineered and control ABPC ADCs specific for TROP2 as described herein. After treatment, cells are transferred to a 96-round bottom plate, and the plate is centrifuged at 400 rcf for 2 min to decant supernatant. Decanted cells are stained with Live/Dead eFluor 660. Cells are then centrifuged and washed with FACS buffer (PBS with 2% FBS), after which cell cycle distribution is analyzed with a BD Cycletest™ Plus DNA Kit (cat #340242). Briefly, cells are re-suspended in 76 μl Solution A and incubated for 10 min at room temperature. 61 μL Solution B is then added, and cells are incubated for another 10 min at room temperature. Finally, 61 μL of cold Solution C is added, and cells are again incubated for 10 min at room temp. Immediately after the last incubation step, cells are analyzed by flow cytometry (without washing) at a flow rate of 10 μL/sec. Increased G2/M-phase arrest can be observed with exposure to pH-engineered ADCs specific for TROP2 as compared to control ABPC ADC specific for TROP2.

For DNA-damaging toxins (e.g., pyrrolobenzodiazepine or “PBD”), DNA damage is assessed by measuring the phosphorylated histone H2AX (γH2AX). H2AX is normally phosphorylated in response to double-strand breaks in DNA; however, increased levels γH2AX may also be observed as a result of treatment with DNA-cross-linking toxins such as PBD or cisplatin (Huang, X. et al. 2004, Cytometry Part A 58A, 99-110). TROP2+ cells are treated with pH-engineered and control ABPC ADCs specific for TROP2 as described herein. After treatment, cells are rinsed with PBS, and then fixed in suspension in 1% methanol-free formaldehyde (Polysciences, Warrington, PA) in PBS at 0° C. for 15 min. Cells are resuspended in 70% ethanol for at least 2 h at −20° C. Cells are then washed twice in PBS and suspended in 0.2% Triton X-100 (Sigma) in a 1% (w/v) solution of BSA (Sigma) in PBS for 30 min to suppress nonspecific Ab binding. Cells are centrifuged again (200 g, 5 min) and the cell pellet is suspended in 100 μL of 1% BSA containing 1:800 diluted anti-histone γH2AX polyclonal Ab (Trevigen, Gaithersburg, MD). The cells are then incubated overnight at 4° C., washed twice with PBS, and resuspended in 100 μL of 1:30 diluted FITC-conjugated F(ab′)2 fragment of swine anti-rabbit immunoglobulin (DAKO, Carpinteria, CA) for 30 min at room temperature in the dark. The cells are then counterstained with 5 μg/mL of PI (Molecular Probes, Eugene, OR) dissolved in PBS containing 100 μg/mL of DNase-free RNase A (Sigma), for 20 min at room temperature. Cellular fluorescence of the FITC γH2AX signal and the PI counterstain are measured using flow cytometry using methods known in the art. When comparing cells within the same stage of the cell cycle (based on total DNA content), treated TROP2+ cells can be observed to have an increased FITC γH2AX signal relative to untreated TROP2+ cells (which serve as a baseline). Furthermore, TROP2+ cells treated with pH-engineered ADCs specific for TROP2 can be observed to have a greater increase in levels of γH2AX over baseline than cells treated with a control ABPC ADC specific for TROP2. In addition to the γH2AX assay, DNA cross-linking can be more directly assessed with a Comet assay (Chandna, S. (2004) Cytometry 61A, 127-133).

In addition, as disclosed herein, pH-engineered and control ABPCs can be assayed using the methods in this example without direct conjugation by performing a secondary ADC assay instead of using primary conjugated ADCs.

### Example 6. Demonstration of Increased or Decreased Half-Life of pH-Engineered ABPCs Specific for TROP2 as Compared to a Control ABPC Specific for TROP2

One of the surprising aspects of the pH-engineered ABPCs specific for TROP2 described by the invention can be their ability to facilitate increased dissociation of ABPCs from the TROP2 within the endosome or lysosome resulting in a decreased or increased serum half-life relative to control ABPCs specific for TROP2 or ABPCs that are not specific for TROP2. To demonstrate these properties, a series of animal studies in mice and/or monkeys is performed using pH-engineered ABPC specific for TROP2 and control ABPC specific for TROP2 using methods known to the art (e.g., Gupta, P., et al. (2016), mAbs, 8:5, 991-997). Briefly, to conduct mouse studies, a single intravenous bolus (e.g., 5 mg/kg) of either pH-engineered ABPC specific for TROP2 or control ABPC specific for TROP2 is administered via tail vein to two groups of NOD SCID mice (e.g. Jackson Labs NOD.CB17-Prkdcscid/J Stock No: 001303) xenografted with a TROP2+ cell line (e.g., as described herein). Xenografted mice are prepared by growing 1-5 million TROP2+ cells in vitro and inoculating subcutaneously into the right flank of the mouse. Tumors are size matched at 300 mm³. Measurements of the length (L) and width (W) of the tumors are taken via electronic caliper and the volume is calculated according to the following equation: V=L×W\hat{ }2/2. Blood samples are collected via retro-orbital bleeds from each group at each of the following time points: 15 m, 30 m, 1 h, 8 h, 24 h, and 3 d, 7 d, 10 d, 14 d, 17 d, 21 d, and 28 d. Samples are processed to collect serum, and antibody concentrations are quantified using ELISA or other methods known to the art (e.g., PAC assay or MAC assay; Fischer, S. K. et al. (2012), mAbs, 4:5, 623-631, utilizing, e.g., anti-human Fc antibody Jackson ImmunoResearch Labs, Cat #109-006-006). Antibody concentrations of pH-engineered ABPC specific for TROP2 and control ABPC specific for TROP2 are plotted as a function of time. Upon analysis of the data, it can be observed that the pH-engineered ABPC specific for TROP2 has a significantly longer or shorter serum half-life relative to control ABPC specific for TROP2. If the pH-engineered and control ABPCs specific for TROP2 are cross-reactive with the mouse homolog of TROP2, a similar experiment can be repeated with non-xenografted mice.

Optionally, if the pH-engineered and control ABPCs specific for TROP2 are cross-reactive with the cynomolgus monkey homolog of TROP2, a similar experiment can be performed on monkeys (e.g., cynomolgus monkeys). An equal number of male and female monkeys (e.g., n=1-2 each) are administered a bolus of either pH-engineered ABPC specific for TROP2 or control ABPC specific for TROP2 at a dose of, e.g., 1 mg/kg via saphenous vein injection. Alternatively, several different doses of TROP2-binding protein are administered across a group of several monkeys. Blood samples are collected via the peripheral vein or femoral vein at intervals similar to those described above, and analyzed for the presence of either pH-engineered ABPC specific for TROP2 or control ABPC specific for TROP2 using methods known to the art (e.g., ELISA). Upon analysis of the data, it can be observed that pH-engineered ABPC specific for TROP2 has a significantly longer or shorter serum half-life relative to control ABPC specific for TROP2. In some cases, this effect is observed only in certain doses, whereas in others it is observed across doses. In particular cases, increased half-life is desirable, especially where less frequent patient dosing is advantageous.

In addition, the half-life of pH-engineered and control ABPC ADCs specific for TROP2 can be assessed using the above methods by substituting pH-engineered and control ABPC ADCs specific for TROP2 for the pH-engineered and control ABPCs specific for TROP2 (i.e., studying the ABPCs after conjugation to a drug or toxin, as described herein).

### Example 7. Increased Potency of pH-Engineered ADCs Specific for TROP2 vs. A Control ABPC ADC Specific for TROP2 in Mouse Xenograft Models

The enhanced anti-tumor activity of the pH-engineered ADCs specific for TROP2 against TROP2+ tumors can be demonstrated in a subcutaneous xenograft model of TROP2+ cells. For the experiments, 1-5 million TROP2+ cells are grown in vitro and inoculated subcutaneously per mouse into the right flank of female immunodeficient (e.g., SCID-Beige or NOD scid) mice. Tumors are size matched at 100-200 mm3, and dosed intraperitoneally (IP) (1 dose given every ~4-7 days for a total of ~2-6 doses). Measurements of the length (L) and width (W) of the tumors are taken via electronic caliper and the volume is calculated according to the following equation: V=L×W\hat{ }2/2. A bolus (e.g., 5 mg/kg) of either pH-engineered ADC specific for TROP2 or control ABPC ADC specific for TROP2 is administered via tail vein. Tumor growth inhibition (TGI) and tumor growth delay (TGD) and survival are significantly improved with administration of pH-engineered ADC specific for TROP2 compared to administration of control ABPC ADC specific for TROP2 at the same regimen.

Optionally, spread of tumor cells into the various tissues is determined in sacrificed animals. Metastasis is measured according to Schneider, T., et al., Clin. Exp. Metas. 19 (2002) 571-582. Briefly, tissues are harvested and human Alu sequences are quantified by real-time PCR. Higher human DNA levels, quantified by real-time PCR, correspond to higher levels of metastasis. Levels of human Alu sequences (correlating to invasion of tumor cells into secondary tissue) are significantly lower in animals treated with pH-engineered ADC specific for TROP2, corresponding to reduced metastasis, compared to mice treated with control ABPC ADC specific for TROP2 at the same regimen. Alternatively, the enhanced anti-tumor activity of the pH-engineered ADC specific for TROP2 can be shown in TROP2+ patient-derived xenograft models.

### Example 8. Creation of a pH-Engineered Bispecific TROP2 Bispecific ABPC and Demonstration of Exemplary Properties as Compared to a Control Bispecific ABPC

To create a pH-engineered ABPC specific for TROP2 with modified toxicity and internalization properties, a bispecific antibody that binds two different epitopes on TROP2 is constructed. It is known in the art that biparatopic antibodies can show increased antigen-dependent internalization, and are therefore useful for applications such as antibody-drug conjugates (e.g., see Li et al (2016) A Biparatopic HER2-Targeting Antibody-Drug Conjugate Induces Tumor Regression in Primary Models Refractory to or Ineligible for HER2-Targeted Therapy, Cancer Cell 29:117-29). Briefly, a pH-engineered TROP2×TROP2 bispecific, biparatopic ABPC specific for TROP2 is assembled using light chain/heavy chain pairs from two different pH-engineered ABPCs specific for TROP2, each of which binds a distinct epitope on TROP2 that does not overlap with the other epitope. A set of pH-engineered ABPCs specific for TROP2 that bind non-overlapping epitopes are discovered, e.g., using the methods described herein, or others known to one of ordinary skill in the art. Briefly, two binders are selected on the basis that they bind substantially different epitopes on TROP2, as determined by, e.g., a binding competition assay as in Abdiche Y N et al (2009) Exploring blocking assays using Octet, ProteOn, and Biacore biosensors, Anal Biochem 386:172-80. Alternatively, briefly, as described herein, cell culture supernatants of cells transfected with a first ABPC specific for TROP2 are normalized to an antibody expression level of 50 μg/mL, and captured on an anti-human Fc sensor (Forte Bio). A baseline is established using 1× kinetics buffer (Forte Bio), and the sensor is associated with 50 nM of TROP2 in 1×PBS (that has been mixed and pre-incubated for 30 min at 37° C. with a second ABPC specific for TROP2 transfection supernatant or the first ABPC specific for TROP2 transfection supernatant, both normalized to 50 ug/mL) at pH 7.4 for 300 sec to generate an association curve. If the association rate in the presence of the second ABPC specific for TROP2 is significantly faster (as calculated by the instrument software, or as seen by an elevated level of association over time) than the association rate in the presence of the first ABPC specific for TROP2, then the second ABPC specific for TROP2 is deemed to bind a non-overlapping epitope of TROP2. Optionally, each antibody is screened for its internalization properties when bound to its epitope on a cell expressing TROP2, and well-internalizing antibodies are selected. Assays for determining the internalization rate of a molecule present on the surface of a cell are known to the art. See, e.g., Wiley et al. (1991) J. Biol. Chem. 266; 11083-11094; and Sorkin and Duex (2010) Curr. Protoc. Cell Biol. Chapter, Unit-15.14; Vainshtein et al. (2015) Pharm Res. 32: 286-299. Once selected, heavy and light chain constructs with engineered mutations for heavy and light chain pairing (Spiess et al., “Alternative molecular formats and therapeutic applications of bispecific antibodies,” 2015) are synthesized for both arms. Bispecific ABPCs specific for TROP2 are produced by co-expression of corresponding heavy and light chain plasmids in, e.g., Expi293 cells. Cell culture supernatants are harvested and subjected to Protein A purification. Heterodimeric ABPCs specific for TROP2 are separated from homodimeric species via additional purification steps such as ion exchange chromatography, hydrophobic interaction chromatography, and mixed mode chromatography. The purified pH-engineered TROP2×TROP2 bispecific, biparatopic ABPCs specific for TROP2 are characterized via mass spectrometry to confirm the purity and absence of homodimeric species and size exclusion chromatography to confirm the presence of monomeric antigen-binding protein construct species. For the product antibody, binding to the TROP2 is confirmed via Biacore analysis. Other methods of bispecific antibody production are known to the art and could also be used to create a bispecific antibody, e.g., the TROP2×TROP2 bispecific, biparatopic ABPCs specific for TROP2 described herein (e.g., Labrijn et al (2014) “Controlled Fab-arm exchange for the generation of stable bispecific IgG1” Nature Protocols 9:2450-2463, accessed at Nature's website, as would be apparent to one of ordinary skill in the art. Alternatively, instead of a TROP2×TROP2 ABPC specific for TROP2, a pH-engineered TROP2×BINDER ABPC specific for TROP2 can be constructed using similar methods apparent to one skilled in the art, where BINDER is any antibody that has been published in the art or discovered using methods like those herein or those known in the art (e.g., display-based or immunization-based methods).

Next, exemplary properties of pH-engineered TROP2×TROP2 ABPCs specific for TROP2 can be demonstrated using the methods described herein, with the appropriate control being a control ABPC monospecific or bispecific ABPC specific for TROP2. Briefly, it can be shown that, as compared to a control, the pH-engineered TROP2×TROP2 ABPCs specific for TROP2: a) bind in a pH-dependent manner to cells, e.g., bind at a neutral pH but not an acidic pH and b) release from cells in a pH-dependent manner, e.g. bind at a neutral pH and release at an acidic pH and c) show enhanced endolysosomal accumulation in TROP2+ cells and d) show increased TROP2 antigen density after exposure to TROP2+ cells and e) when conjugated to a toxin, show increased cytotoxicity to TROP2+ cells and f) when conjugated to a toxin, show increased toxin liberation when incubated with TROP2+ cells and g) show decreased half-life when exposed to TROP2 antigen in a relevant animal model and h) when conjugated to a toxin, show increased efficacy in a mouse xenograft model of TROP2+ cells. Similarly, the exemplary properties of pH-engineered TROP2×BINDER ABPCs specific for TROP2 can be demonstrated using the methods described herein, with the appropriate control being a control ABPC TROP2×BINDER bispecific ABPC specific for TROP2.

### Example 9. Construction and Screening of pH-Engineered TROP2 ABPCs

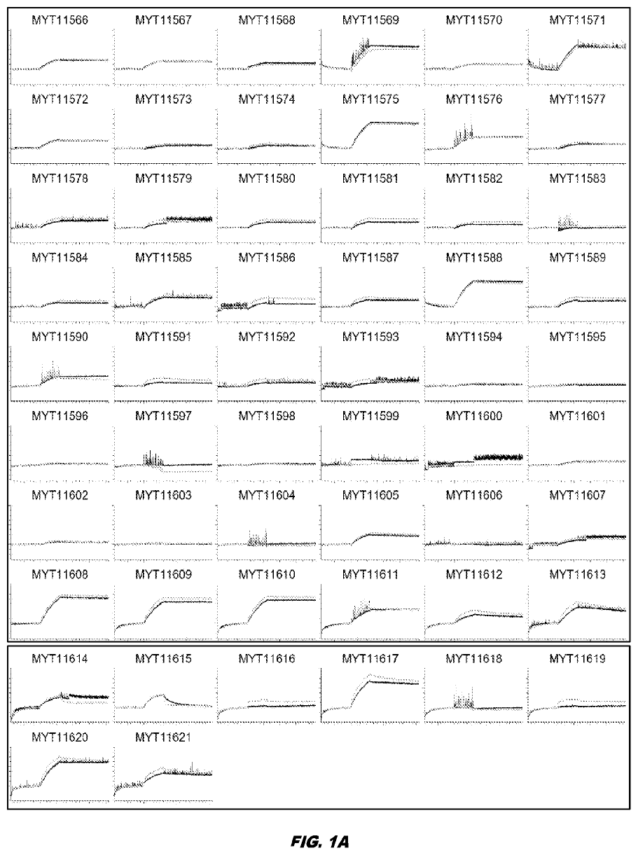

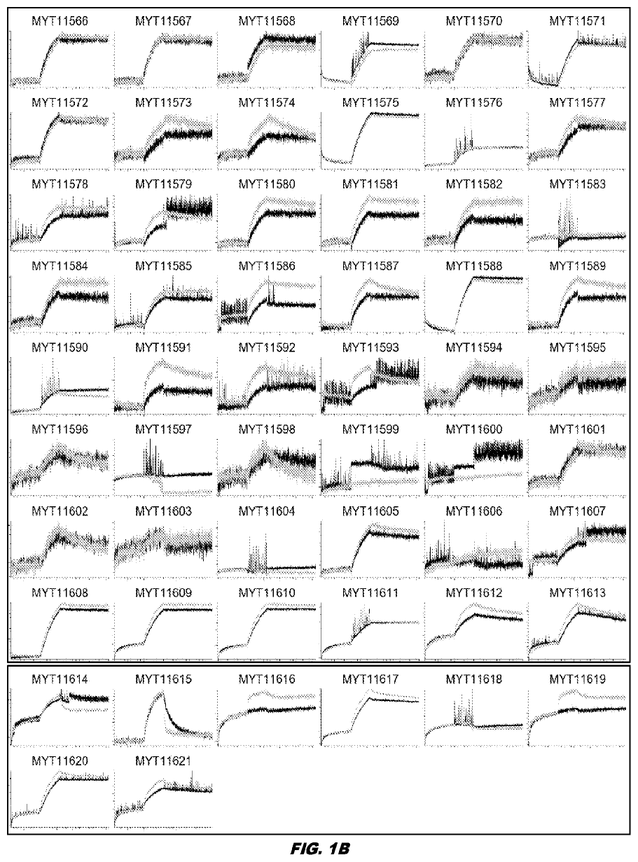

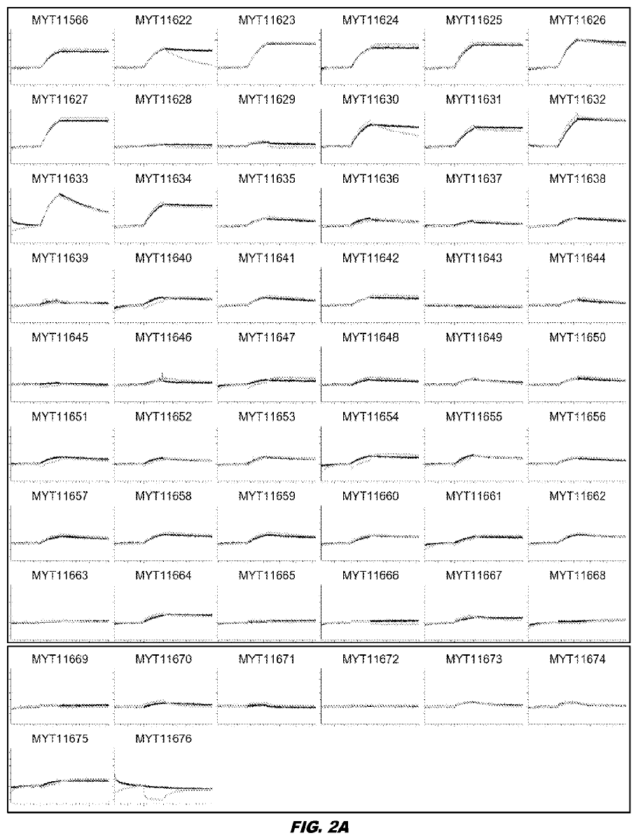

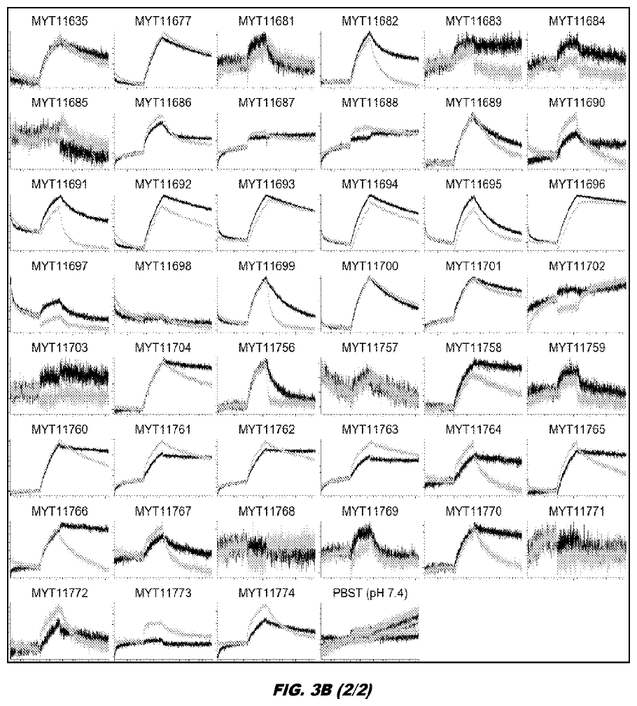

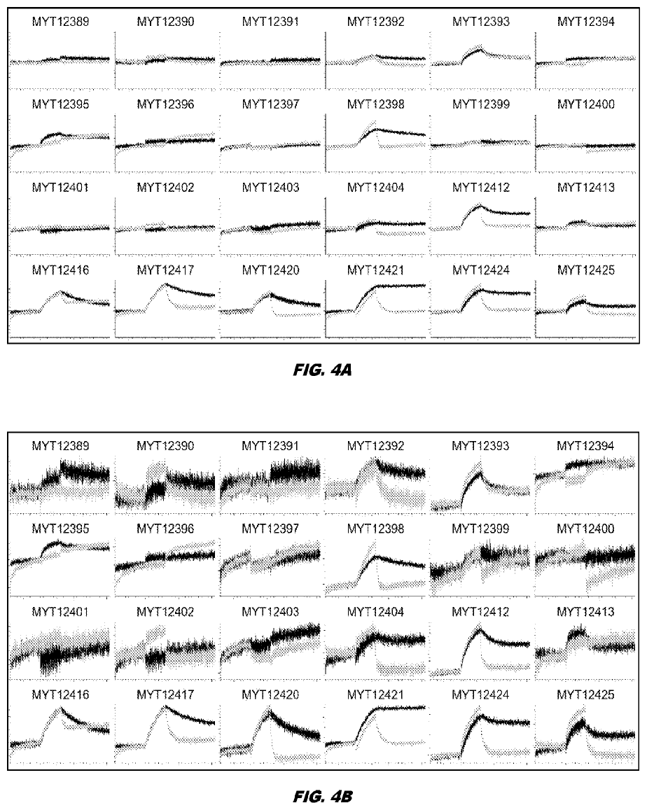

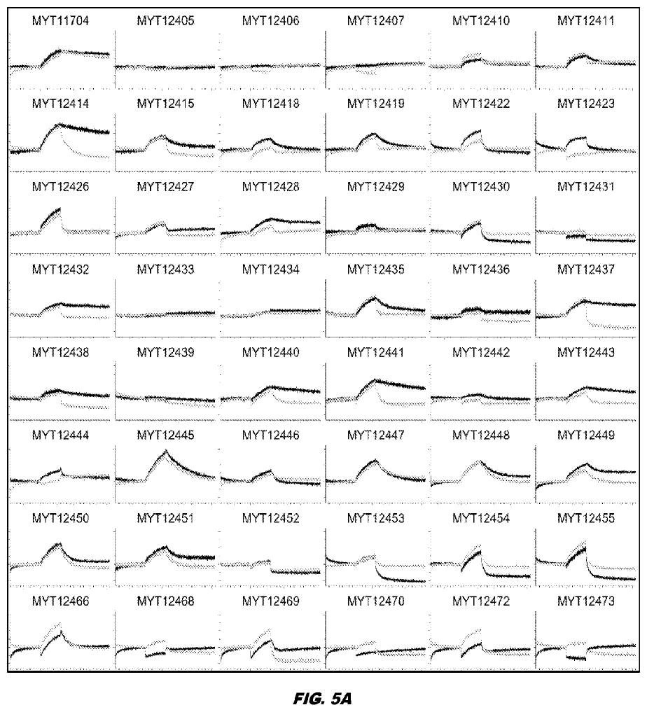

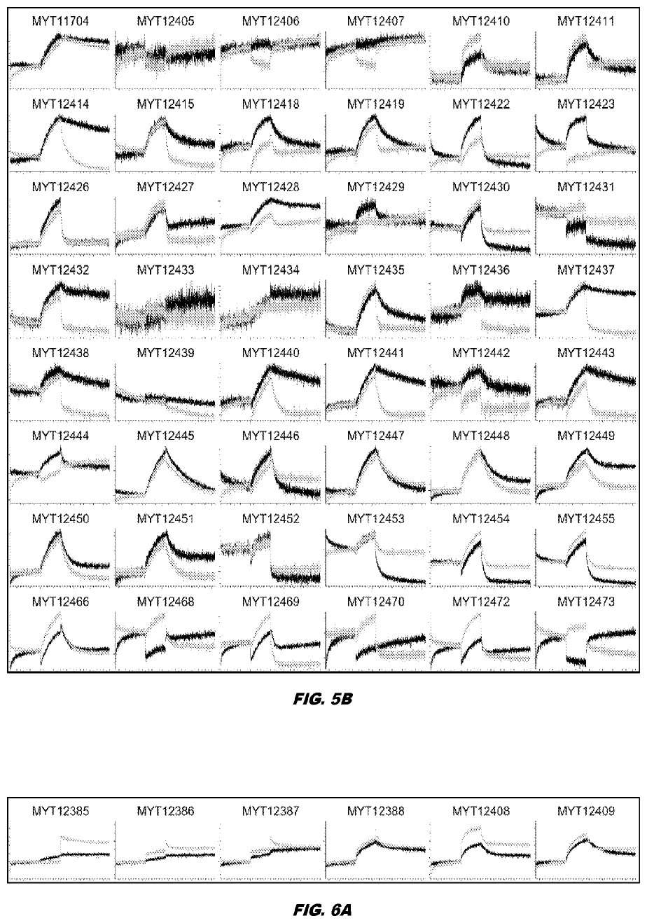

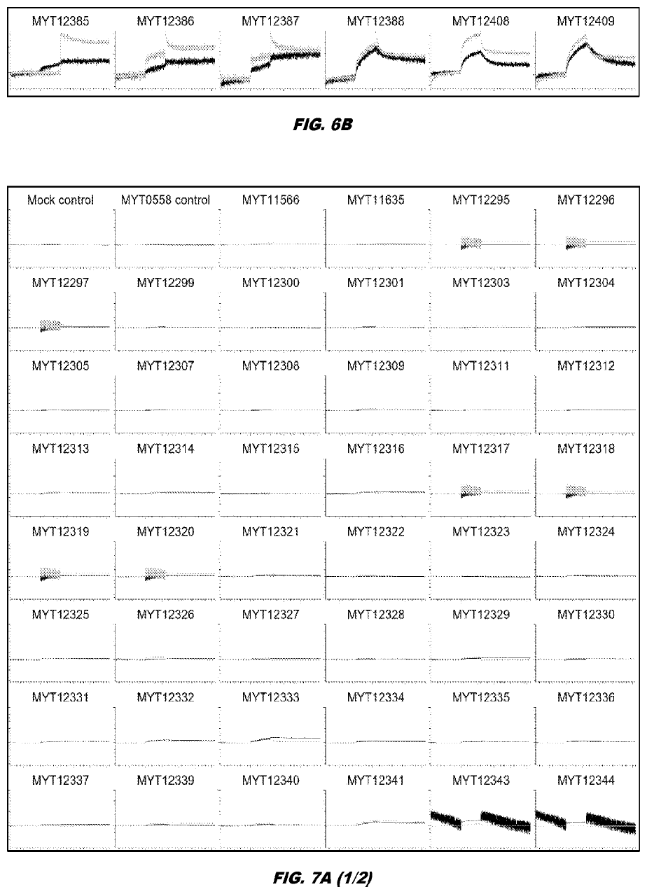

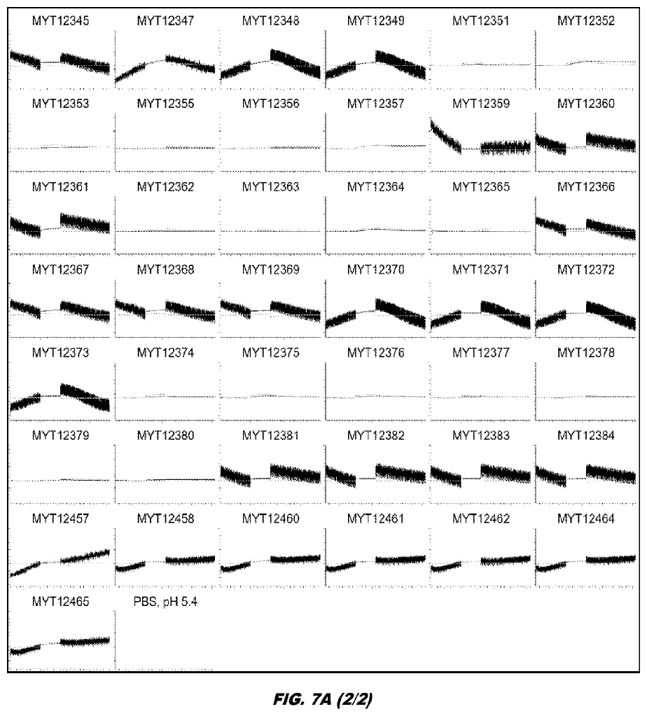

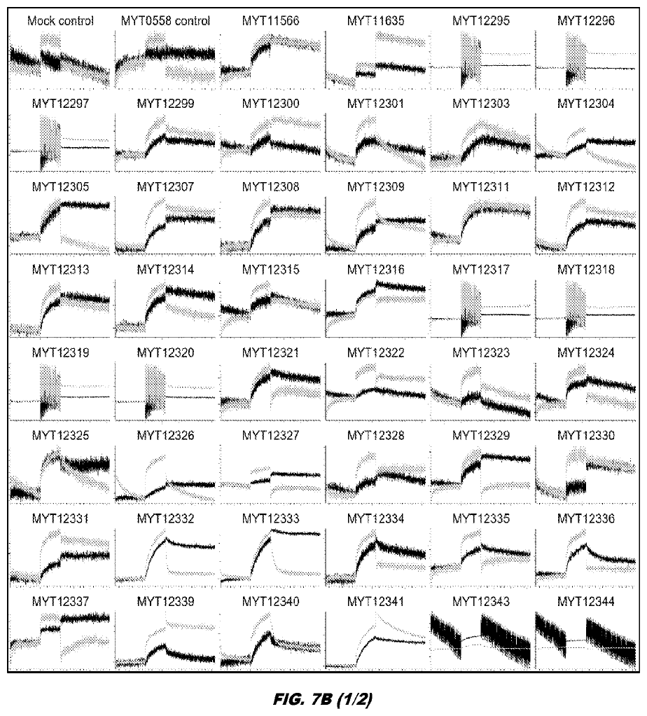

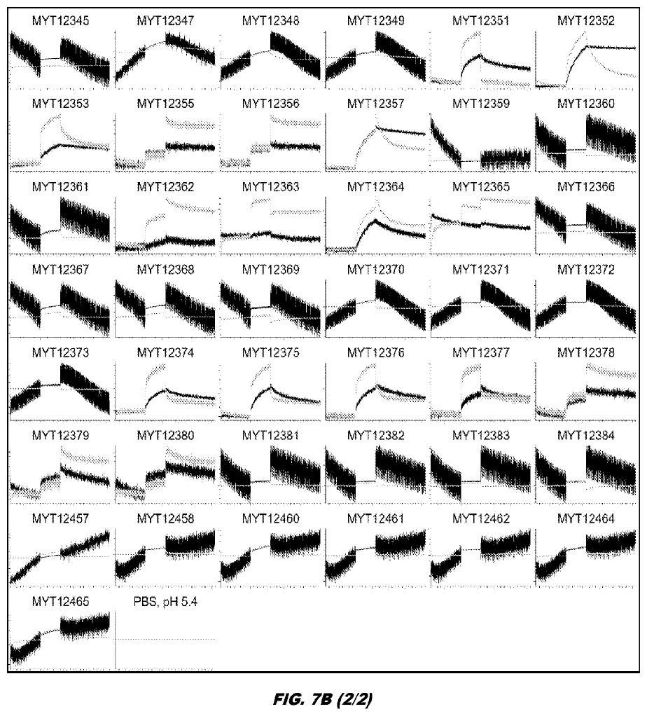

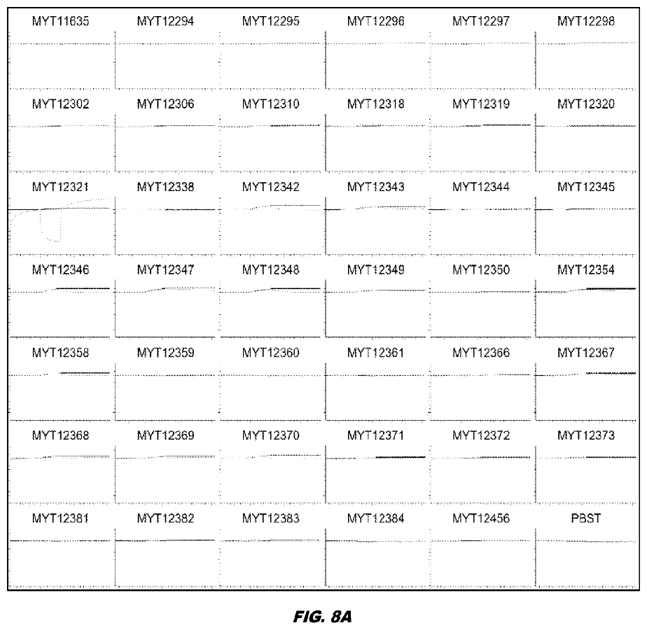

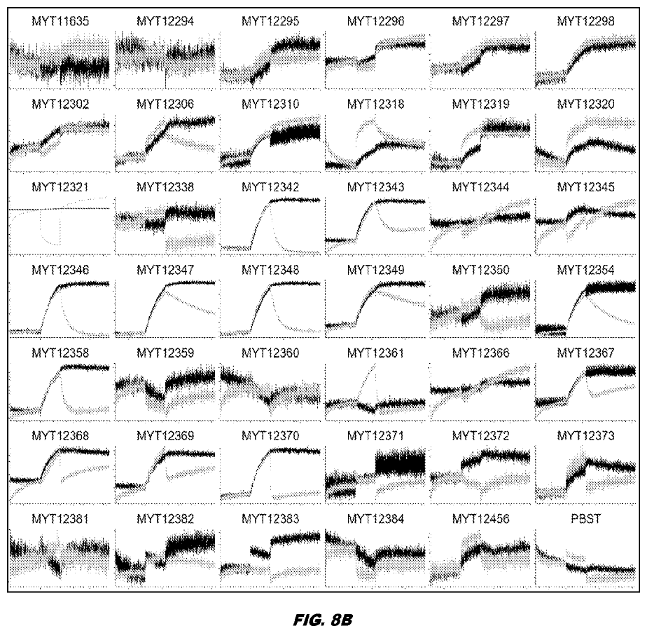

Multiple TROP2-binding monoclonal antibodies have been described in the literature and can be used as a template for engineering pH-dependent TROP2-binding proteins, including pH-dependent TROP2-binding monoclonal antibodies. We selected sacituzumab(HCVD SEQ ID NO: 4, LCVD SEQ ID NO: 5) as a TROP2-binding monoclonal antibody for pH engineering via histidine scanning. Briefly, CDRs in the heavy and light chains were identified using the methods described by Kabat et al (Kabat et al. (1992) Sequences of Proteins of Immunological Interest, DIANE publishing) and IMGT (Lefranc M P (1999) “The IMGT unique numbering for Immunoglobulins, T cell receptors and Ig-like domains” The Immunologist 7, 132-136), and for each CDR, residues falling under either or both Kabat and IMGT CDR definitions were called as CDR residues. To generate pH-dependent sequence variants, individual amino acid residues within the heavy and light chain CDRs were systematically substituted with a histidine, one at a time to produce MYT11567-MYT11607 and MYT11608-MYT11634. In cases where the starting CDR residue was a histidine, it was mutated to an alanine. Antibody variants with only one histidine or alanine mutation in either a heavy or light chain CDR were generated by co-transfection of Expi293 cells with either a) one heavy chain sequence variant, and the corresponding starting ABPC light chain; or b) one light chain sequence variant, and the corresponding starting ABPC heavy chain using methods known to the art. After allowing for four days of protein expression, cell culture supernatants were collected, quantified by SDS-PAGE analysis (DNS), and the pH dependence of the variant was evaluated using biolayer interferometry (BLI) on an Octet RED 96e instrument. Briefly, cell culture supernatants were diluted based on qualitative expression level of the variant determined by visual examination of SDS-PAGE gels, 5 μL of cell culture supernatant was diluted into 195 μL of 1×PBST, pH 7.4 for high expressors, 25 μL of cell culture supernatant was diluted into 175 μL of 1×PBST, pH 7.4 for medium expressors and 100 μL of cell culture supernatant was diluted into 100 μL of 1×PBST, pH 7.4 for low expressors for loading onto the sensor tips. The resulting diluted supernatants were then captured on an anti-human Fc sensor (Forte Bio). A baseline was established using 1×PBST (50 mM Potassium Phosphate Buffer+150 mM NaCl+0.05% Tween 20) pH 7.4, and the sensor was associated with 50 nM of TROP2 (Human TROP2 Protein, His Tag, Acro Biosystems) in 1×PBST pH 7.4 for 120 sec to generate an association curve. In the dissociation phase, the antibody-antigen complex on the sensor was exposed to 1×PBST pH 7.4 or pH 5.4, for 300 sec. Association and dissociation phase curves were examined for the starting ABPC antibody (with no substitutions) and each corresponding antibody variant at pH 5.4 and pH 7.4 to inform on two criteria: a) enhanced dissociation (i.e., higher koff values) at pH 5.4 due to histidine or alanine substitution compared to the starting ABPC, (with no substitutions), and b) reduced dissociation at pH 7.4 (i.e., lower koff values) compared to pH 5.4 in the antibody variant itself and with the starting ABPC (with no substitutions). Heavy and light chain variants that showed either enhanced dissociation at pH 5.4 or reduced dissociation at pH 7.4 or both (as compared to starting ABPC; see FIGS. 1A-11B) were selected for further analysis (e.g., MYT numbers in Table 7 associated with an octet binding results of “I”). It was also noted that while some mutations obliterated TROP2 binding, others were tolerated with little or n change in TROP2 binding kinetics.

Especially because histidine is a large, positively charged amino acid, heavy or light chain variants exhibiting no significant change in binding were noted as positions in the heavy or light chain that may tolerate a wide range of mutations and lead to antibodies with different sequence but similar binding properties, a designation that is not otherwise apparent.

TABLE 7
Sacituzumab variants information, including sequence, protein, and binding data

| | | | | | | | | Octet | |
|---|---|---|---|---|---|---|---|---|---|
| | CDR | HCV | LCV | A Sub | HCV | LCV | SDS | loading | Octet |
| MYT | H-Sub | Sub | Sub | Pos if | SEQ | SEQ | PAGE | result | Binding |
| # | Pos | Pos | Pos | made | ID NO | ID NO | result | (nm) | Result |
| 11566 | n/a | n/a | n/a | | 4 | 5 | Yes | 0.37 | n/a |
| 11567 | H1 01 | 26 | n/a | | 12 | 5 | Yes | 0.32 | 2 |
| 11568 | H1 02 | 27 | n/a | | 13 | 5 | Yes | 0.26 | 2 |
| 11569 | H1 03 | 28 | n/a | | 14 | 5 | Yes | 1.56 | 2 |
| 11570 | H1 04 | 29 | n/a | | 15 | 5 | Yes | 0.24 | 1 |
| 11571 | H1 05 | 30 | n/a | | 16 | 5 | Yes | 1.67 | 2 |
| 11572 | H1 06 | 31 | n/a | | 17 | 5 | Yes | 0.33 | 2 |
| 11573 | H1 07 | 32 | n/a | | 18 | 5 | Yes | 0.21 | 1 |
| 11574 | H1 08 | 33 | n/a | | 19 | 5 | Yes | 0.31 | 1 |
| 11575 | H1 09 | 34 | n/a | | 20 | 5 | Yes | 1.29 | 2 |
| 11576 | H1 10 | 35 | n/a | | 21 | 5 | Yes | 0.43 | 2 |
| 11577 | H2 01 | 50 | n/a | | 22 | 5 | Yes | 0.31 | 1 |
| 11578 | H2 02 | 51 | n/a | | 23 | 5 | Yes | 0.26 | 2 |
| 11579 | H2 03 | 52 | n/a | | 24 | 5 | Yes | 0.19 | 1 |
| 11580 | H2 04 | 53 | n/a | | 25 | 5 | Yes | 0.27 | 1 |
| 11581 | H2 05 | 54 | n/a | | 26 | 5 | Yes | 0.35 | 2 |
| 11582 | H2 06 | 55 | n/a | | 27 | 5 | Yes | 0.20 | 2 |
| 11583 | H2 07 | 56 | n/a | | 28 | 5 | Yes | 0.23 | 2 |
| 11584 | H2 08 | 57 | n/a | | 29 | 5 | Yes | 0.26 | 2 |
| 11585 | H2 09 | 58 | n/a | | 30 | 5 | Yes | 0.38 | 2 |
| 11586 | H2 10 | 59 | n/a | | 31 | 5 | Yes | 0.31 | 2 |
| 11587 | H2 11 | 60 | n/a | | 32 | 5 | Yes | 0.26 | 1 |
| 11588 | H2 12 | 61 | n/a | | 33 | 5 | Yes | 1.37 | 1 |
| 11589 | H2 13 | 62 | n/a | | 34 | 5 | Yes | 0.29 | 1 |
| 11590 | H2 14 | 63 | n/a | | 35 | 5 | Yes | 0.34 | 1 |
| 11591 | H2 15 | 64 | n/a | | 36 | 5 | Yes | 0.15 | 1 |
| 11592 | H2 16 | 65 | n/a | | 37 | 5 | Yes | 0.16 | 1 |
| 11593 | H2 17 | 66 | n/a | | 38 | 5 | Yes | 0.16 | 1 |
| 11594 | H3 01 | 97 | n/a | | 39 | 5 | Yes | 0.13 | 2 |
| 11595 | H3 02 | 98 | n/a | | 40 | 5 | Yes | 0.20 | 2 |
| 11596 | H3 03 | 99 | n/a | | 41 | 5 | Yes | 0.28 | 1 |
| 11597 | H3 04 | 100 | n/a | | 42 | 5 | Yes | 0.29 | 3 |
| 11598 | H3 05 | 101 | n/a | | 43 | 5 | Yes | 0.16 | 1 |
| 11599 | H3 06 | 102 | n/a | | 44 | 5 | Yes | 0.24 | 3 |
| 11600 | H3 07 | 103 | n/a | | 45 | 5 | Yes | 0.23 | 3 |
| 11601 | H3 08 | 104 | n/a | | 46 | 5 | Yes | 0.23 | 1 |
| 11602 | H3 09 | 105 | n/a | | 47 | 5 | Yes | 0.18 | 1 |
| 11603 | H3 10 | 106 | n/a | | 48 | 5 | Yes | 0.27 | 3 |
| 11604 | H3 11 | 107 | n/a | | 49 | 5 | Yes | 0.21 | 3 |
| 11605 | H3 12 | 108 | n/a | | 50 | 5 | Yes | 0.47 | 1 |
| 11606 | H3 13 | 109 | n/a | | 51 | 5 | Yes | 0.38 | 3 |
| 11607 | H3 14 | 110 | n/a | | 52 | 5 | Yes | 0.22 | 2 |
| 11608 | L1 01 | n/a | 24 | | 4 | 53 | Yes | 1.47 | 2 |
| 11609 | L1 02 | n/a | 25 | | 4 | 54 | Yes | 0.94 | 2 |
| 11610 | L1 03 | n/a | 26 | | 4 | 55 | Yes | 1.05 | 2 |
| 11611 | L1 04 | n/a | 27 | | 4 | 56 | Yes | 0.67 | 2 |
| 11612 | L1 05 | n/a | 28 | | 4 | 57 | Yes | 0.38 | 1 |
| 11613 | L1 06 | n/a | 29 | | 4 | 58 | Yes | 1.11 | 1 |
| 11614 | L1 07 | n/a | 30 | | 4 | 59 | Yes | 0.42 | 1 |
| 11615 | L1 08 | n/a | 31 | | 4 | 60 | Yes | 1.71 | 1 |
| 11616 | L1 09 | n/a | 32 | | 4 | 61 | Yes | 0.38 | 1 |
| 11617 | L1 10 | n/a | 33 | | 4 | 62 | Yes | 1.17 | 1 |
| 11618 | L1 11 | n/a | 34 | | 4 | 63 | Yes | 0.85 | 3 |
| 11619 | L2 01 | n/a | 50 | | 4 | 64 | Yes | 0.56 | 1 |
| 11620 | L2 02 | n/a | 51 | | 4 | 65 | Yes | 1.09 | 1 |
| 11621 | L2 03 | n/a | 52 | | 4 | 66 | Yes | 0.40 | 1 |
| 11622 | L2 04 | n/a | 53 | | 4 | 67 | Yes | 0.65 | 1 |
| 11623 | L2 05 | n/a | 54 | | 4 | 68 | Yes | 0.75 | 2 |
| 11624 | L2 06 | n/a | 55 | | 4 | 69 | Yes | 0.56 | 2 |
| 11625 | L2 07 | n/a | 56 | | 4 | 70 | Yes | 0.68 | 2 |
| 11626 | L3 01 | n/a | 89 | | 4 | 71 | Yes | 0.80 | 1 |
| 11627 | L3 02 | n/a | 90 | | 4 | 72 | Yes | 0.77 | 2 |
| 11628 | L3 03 | n/a | 91 | A | 4 | 73 | Yes | 0.87 | 1 |
| 11629 | L3 04 | n/a | 92 | | 4 | 74 | Yes | 0.55 | 1 |
| 11630 | L3 05 | n/a | 93 | | 4 | 75 | Yes | 0.69 | 1 |
| 11631 | L3 06 | n/a | 94 | | 4 | 76 | Yes | 0.64 | 1 |
| 11632 | L3 07 | n/a | 95 | | 4 | 77 | Yes | 1.40 | 1 |
| 11633 | L3 08 | n/a | 96 | | 4 | 78 | Yes | 1.36 | 1 |
| 11634 | L3 09 | n/a | 97 | | 4 | 79 | Yes | 0.70 | 1 |

For each such table, the “MYT #” provides an identifier for each antigen binding protein (ABP), and the values from each column (reading along each row) provide characteristics and data associated with each ABP. “Sub Pos” indicates the amino acid position in the indicated heavy chain variable (HCV) or light chain variable (LCV) SEQ ID NO, and “CDR H” and “CDR L” mean, respectively, the indicated heavy or light chain CDR (e.g., 1^{st}, 2^{nd}, or 3^{rd}). The “SDS PAGE Result” indicates whether a band consistent with full length IgG antibody was observed on an SDS PAGE gel and the “Octet Loading Result” is the thicknesses of the layer (in nm) that was deposited onto the Octet device's sensor surface. Because the anti-human capture (AHC) sensors were coated with an anti-human Fc antibody, they should be highly specific for the ABPs under evaluation, and reliable data has been observed coming from as little as 0.1 nm of loading. And finally, the “Octet Binding Result” indicates the binding behavior of the variant vs. its corresponding parental antibody: 1=pH dependent; 2=No change; 3=No binding.

### Example 10. Construction and Screening of pH-Engineered TROP2 ABPCs

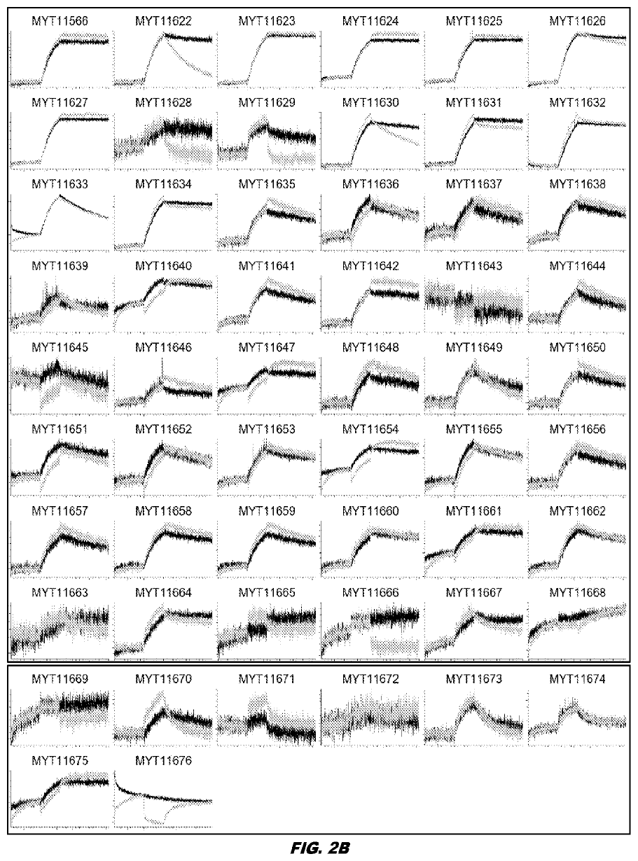

Using the methods detailed in Example 9, desirable mutations may be systematically combined two or more at a time (i.e., to produce double, triple, quadruple, etc., HC and LC histidine (or alanine) substitution variants). Heavy and light chain combination variants showing either enhanced dissociation at pH 5.4 or reduced dissociation at pH 7.4 or both (as compared to the starting ABPC; see, e.g., FIGS. 2A-B and 3A-B), were selected for further analysis. It was also noted that while some mutations obliterated TROP2 binding, others were tolerated with little or no change in TROP2 binding kinetics. For all octet plots depicted herein, grey and black lines correspond to pH 5.4 and pH 7.4, respectively.

TABLE 8
Datopotamab variants information, including sequence, protein, and binding data

| | CDR | HCV | LCV | A Sub | HCV | LCV | SDS | Octet | Octet |
|---|---|---|---|---|---|---|---|---|---|
| MYT | H-Sub | Sub | Sub | Pos if | SEQ | SEQ | PAGE | loading | Binding |
| # | Pos | Pos | Pos | made | ID NO | ID NO | result | result | Result |
| 11635 | n/a | n/a | n/a | | 80 | 81 | Yes | 0.26 | n/a |
| 11636 | H1 01 | 26 | n/a | | 88 | 81 | Yes | 0.24 | 2 |
| 11637 | H1 02 | 27 | n/a | | 89 | 81 | Yes | 0.22 | 2 |
| 11638 | H1 03 | 28 | n/a | | 90 | 81 | Yes | 0.28 | 2 |
| 11639 | H1 04 | 29 | n/a | | 91 | 81 | Yes | 0.19 | 1 |
| 11640 | H1 05 | 30 | n/a | | 92 | 81 | Yes | 0.26 | 2 |
| 11641 | H1 06 | 31 | n/a | | 93 | 81 | Yes | 0.29 | 2 |
| 11642 | H1 07 | 32 | n/a | | 94 | 81 | Yes | 0.29 | 2 |
| 11643 | H1 08 | 33 | n/a | | 95 | 81 | Yes | 0.22 | 3 |
| 11644 | H1 09 | 34 | n/a | | 96 | 81 | Yes | 0.24 | 2 |
| 11645 | H1 10 | 35 | n/a | | 97 | 81 | Yes | 0.09 | 3 |
| 11646 | H2 01 | 50 | n/a | | 98 | 81 | Yes | 0.24 | 2 |
| 11647 | H2 02 | 51 | n/a | | 99 | 81 | Yes | 0.19 | 2 |
| 11648 | H2 03 | 52 | n/a | | 100 | 81 | Yes | 0.22 | 2 |
| 11649 | H2 04 | 53 | n/a | | 101 | 81 | Yes | 0.23 | 1 |
| 11650 | H2 05 | 54 | n/a | A | 102 | 81 | Yes | 0.25 | 2 |
| 11651 | H2 06 | 55 | n/a | | 103 | 81 | Yes | 0.23 | 1 |
| 11652 | H2 07 | 56 | n/a | | 104 | 81 | Yes | 0.20 | 2 |
| 11653 | H2 08 | 57 | n/a | | 105 | 81 | Yes | 0.26 | 2 |
| 11654 | H2 09 | 58 | n/a | | 106 | 81 | Yes | 0.27 | 2 |
| 11655 | H2 10 | 59 | n/a | | 107 | 81 | Yes | 0.30 | 2 |
| 11656 | H2 11 | 60 | n/a | | 108 | 81 | Yes | 0.18 | 2 |
| 11657 | H2 12 | 61 | n/a | | 109 | 81 | Yes | 0.22 | 2 |
| 11658 | H2 13 | 62 | n/a | | 110 | 81 | Yes | 0.28 | 2 |
| 11659 | H2 14 | 63 | n/a | | 111 | 81 | Yes | 0.30 | 2 |
| 11660 | H2 15 | 64 | n/a | | 112 | 81 | Yes | 0.23 | 1 |
| 11661 | H2 16 | 65 | n/a | | 113 | 81 | Yes | 0.21 | 1 |
| 11662 | H2 17 | 66 | n/a | | 114 | 81 | Yes | 0.30 | 2 |
| 11663 | H3 01 | 97 | n/a | | 115 | 81 | Yes | 0.10 | 3 |
| 11664 | H3 02 | 98 | n/a | | 116 | 81 | Yes | 0.23 | 1 |
| 11665 | H3 03 | 99 | n/a | | 117 | 81 | Yes | 0.19 | 3 |
| 11666 | H3 04 | 100 | n/a | | 118 | 81 | Yes | 0.21 | 3 |
| 11667 | H3 05 | 101 | n/a | | 119 | 81 | Yes | 0.20 | 1 |
| 11668 | H3 06 | 102 | n/a | | 120 | 81 | Yes | 0.22 | 3 |
| 11669 | H3 07 | 103 | n/a | | 121 | 81 | Yes | 0.27 | 3 |
| 11670 | H3 08 | 104 | n/a | | 122 | 81 | Yes | 0.23 | 1 |
| 11671 | H3 09 | 105 | n/a | | 123 | 81 | Yes | 0.20 | 1 |
| 11672 | H3 10 | 106 | n/a | | 124 | 81 | Yes | 0.24 | 3 |
| 11673 | H3 11 | 107 | n/a | | 125 | 81 | Yes | 0.21 | 1 |
| 11674 | H3 12 | 108 | n/a | | 126 | 81 | Yes | 0.27 | 1 |
| 11675 | H3 13 | 109 | n/a | | 127 | 81 | Yes | 0.24 | |
| 11676 | H3 14 | 110 | n/a | | 128 | 81 | | 0.06 | 3 |
| 11677 | L1 01 | n/a | 24 | | 80 | 129 | | | 2 |
| 11678 | L1 02 | n/a | 25 | | 80 | 130 | Yes | 1.20 | 2 |
| 11679 | L1 03 | n/a | 26 | | 80 | 131 | Yes | 0.76 | 1 |
| 11680 | L1 04 | n/a | 27 | | 80 | 132 | Yes | 1.70 | 2 |
| 11681 | L1 05 | n/a | 28 | | 80 | 133 | Yes | | 1 |
| 11682 | L1 06 | n/a | 29 | | 80 | 134 | Yes | | 1 |
| 11683 | L1 07 | n/a | 30 | | 80 | 135 | Yes | | 1 |
| 11684 | L1 08 | n/a | 31 | | 80 | 136 | Yes | | 1 |
| 11685 | L1 09 | n/a | 32 | | 80 | 137 | Yes | | 3 |
| 11686 | L1 10 | n/a | 33 | | 80 | 138 | Yes | | 1 |
| 11687 | L1 11 | n/a | 34 | | 80 | 139 | Yes | | 1 |
| 11688 | L2 01 | n/a | 50 | | 80 | 140 | Yes | | 1 |
| 11689 | L2 02 | n/a | 51 | | 80 | 141 | Yes | | 1 |
| 11690 | L2 03 | n/a | 52 | | 80 | 142 | Yes | | 1 |
| 11691 | L2 04 | n/a | 53 | | 80 | 143 | Yes | | 1 |
| 11692 | L2 05 | n/a | 54 | | 80 | 144 | Yes | | 1 |
| 11693 | L2 06 | n/a | 55 | | 80 | 145 | Yes | | 2 |
| 11694 | L2 07 | n/a | 56 | | 80 | 146 | Yes | | 2 |
| 11695 | L3 01 | n/a | 89 | | 80 | 147 | Yes | | 1 |
| 11696 | L3 02 | n/a | 90 | | 80 | 148 | Yes | | 2 |
| 11697 | L3 03 | n/a | 91 | A | 80 | 149 | Yes | | 1 |
| 11698 | L3 04 | n/a | 92 | | 80 | 150 | Yes | | 3 |
| 11699 | L3 05 | n/a | 93 | | 80 | 151 | Yes | | 1 |
| 11700 | L3 06 | n/a | 94 | | 80 | 152 | Yes | | 1 |
| 11701 | L3 07 | n/a | 95 | | 80 | 153 | Yes | | 1 |
| 11702 | L3 08 | n/a | 96 | | 80 | 154 | Yes | | 3 |
| 11703 | L3 09 | n/a | 97 | | 80 | 155 | Yes | | 3 |

### Example 11. Construction and Screening of pH-Engineered TROP2 ABPCs

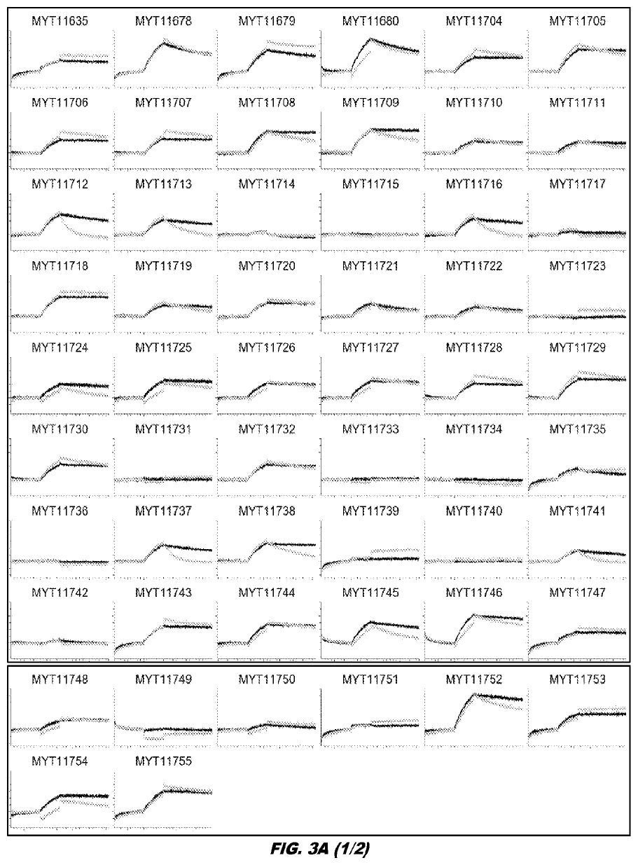

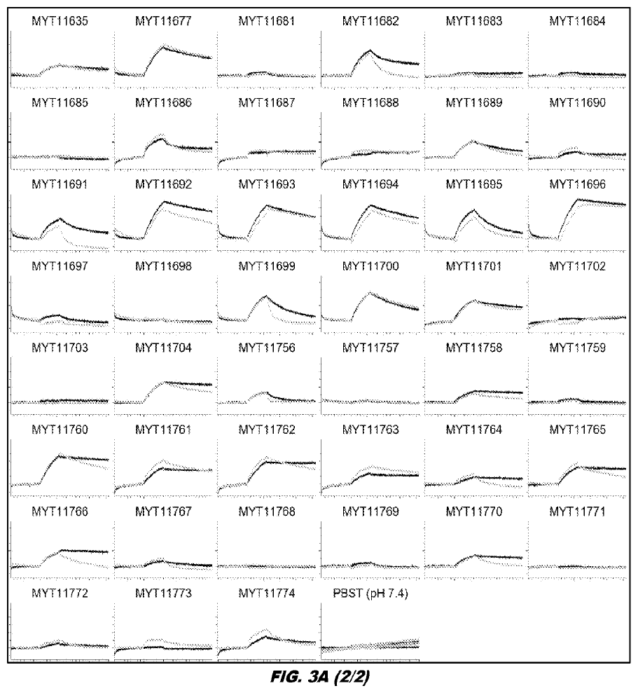

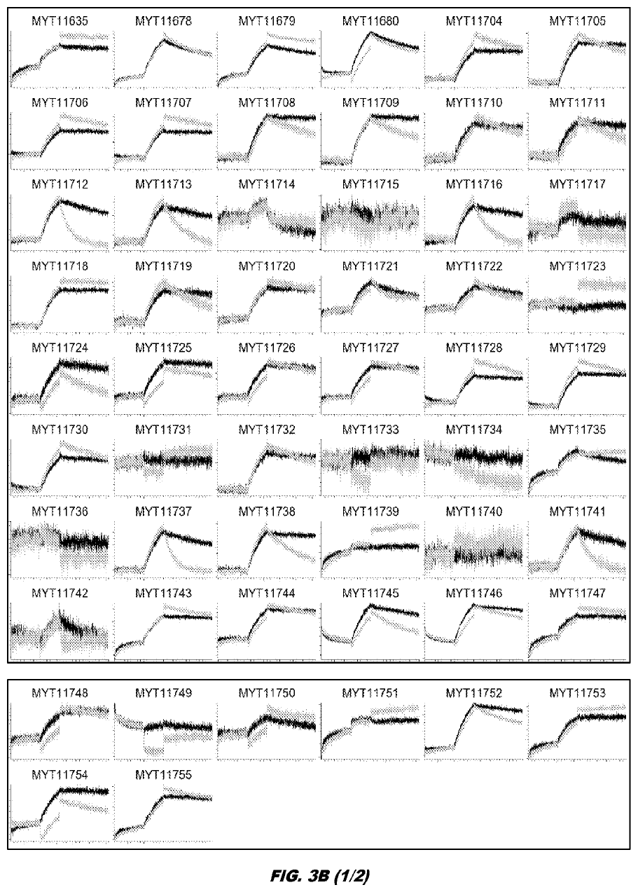

The methods of Examples 9-10 were applied to datopotamab (HCV SEQ ID NO: 80, LCV SEQ ID NO. 81) to produce HC single substitution variants (MYT111636-MYT111676) and LC single substitution variants (MYT11677-MYT11703); and hu7E6SVG (HCV SEQ ID NO: 156, LCV SEQ ID NO: 157) to produce to produce HC single substitution variants (MYT11704-MYT11743) and LC single substitution variants (MYT11744-MYT11774). Variants that show either enhanced dissociation at pH 5.4 or reduced dissociation at pH 7.4, or both are selected for further analysis. FIGS. 3A-B and 4A-B present octet plots for the foregoing variants and their parental antibodies. As indicated by the plots, some mutations obliterated TROP2 binding, while others were tolerated with little or no change in TROP2 binding kinetics.

TABLE 9
hu7E6SVG variants information, including sequence, protein, and binding data

| | CDR | HCV | LCV | A Sub | HCV | LCV | SDS | Octet | Octet |
|---|---|---|---|---|---|---|---|---|---|
| MYT | H-Sub | Sub | Sub | Pos if | SEQ | SEQ | PAGE | loading | Binding |
| # | Pos | Pos | Pos | made | ID NO | ID NO | result | result | Result |
| 11704 | n/a | n/a | n/a | | 156 | 157 | YES | 0.56 | n/a |
| 11705 | H1 01 | 26 | n/a | | 164 | 157 | YES | 0.85 | 1 |
| 11706 | H1 02 | 27 | n/a | | 165 | 157 | YES | 0.56 | 2 |
| 11707 | H1 03 | 28 | n/a | | 166 | 157 | YES | 0.62 | 2 |
| 11708 | H1 04 | 29 | n/a | | 167 | 157 | YES | 0.80 | 1 |
| 11709 | H1 05 | 30 | n/a | | 168 | 157 | YES | 0.84 | 1 |
| 11710 | H1 06 | 31 | n/a | | 169 | 157 | YES | 0.44 | 2 |
| 11711 | H1 07 | 32 | n/a | | 170 | 157 | YES | 0.39 | 2 |
| 11712 | H1 08 | 33 | n/a | | 171 | 157 | YES | 0.70 | 1 |
| 11713 | H1 09 | 34 | n/a | | 172 | 157 | YES | 0.61 | 1 |
| 11714 | H1 10 | 35 | n/a | A | 173 | 157 | YES | 0.47 | 1 |
| 11715 | H2 01 | 50 | n/a | | 174 | 157 | YES | 0.55 | 3 |
| 11716 | H2 02 | 51 | n/a | | 175 | 157 | YES | 0.60 | 1 |
| 11717 | H2 03 | 52 | n/a | | 176 | 157 | YES | 0.61 | 3 |
| 11718 | H2 04 | 53 | n/a | | 177 | 157 | YES | 0.65 | 2 |
| 11719 | H2 05 | 54 | n/a | | 178 | 157 | YES | 0.39 | 1 |
| 11720 | H2 06 | 55 | n/a | | 179 | 157 | YES | 0.49 | 2 |
| 11721 | H2 07 | 56 | n/a | | 180 | 157 | YES | 0.72 | 1 |
| 11722 | H2 08 | 57 | n/a | | 181 | 157 | YES | 0.52 | 1 |
| 11723 | H2 09 | 58 | n/a | | 182 | 157 | YES | 0.58 | 3 |
| 11724 | H2 10 | 59 | n/a | | 183 | 157 | YES | 0.54 | 1 |
| 11725 | H2 11 | 60 | n/a | | 184 | 157 | YES | 0.57 | 2 |
| 11726 | H2 12 | 61 | n/a | | 185 | 157 | YES | 0.53 | 2 |
| 11727 | H2 13 | 62 | n/a | | 186 | 157 | YES | 0.64 | 2 |
| 11728 | H2 14 | 63 | n/a | | 187 | 157 | YES | 0.61 | 2 |
| 11729 | H2 15 | 64 | n/a | | 188 | 157 | YES | 0.70 | 2 |
| 11730 | H2 16 | 65 | n/a | | 189 | 157 | YES | 0.63 | 2 |
| 11731 | H3 01 | 96 | n/a | | 190 | 157 | YES | 1.44 | 3 |
| 11732 | H3 02 | 97 | n/a | | 191 | 157 | YES | 0.60 | 2 |
| 11733 | H3 03 | 98 | n/a | | 192 | 157 | YES | 0.57 | 3 |
| 11734 | H3 04 | 99 | n/a | | 193 | 157 | YES | 0.41 | 3 |
| 11735 | H3 05 | 100 | n/a | | 194 | 157 | YES | 0.57 | 2 |
| 11736 | H3 06 | 101 | n/a | | 195 | 157 | YES | 0.74 | 3 |
| 11737 | H3 07 | 102 | n/a | | 196 | 157 | YES | 0.78 | 1 |
| 11738 | H3 08 | 103 | n/a | | 197 | 157 | YES | 0.64 | 1 |
| 11739 | H3 09 | 104 | n/a | | 198 | 157 | YES | 0.76 | 3 |
| 11740 | H3 10 | 105 | n/a | | 199 | 157 | YES | 0.63 | 3 |
| 11741 | H3 11 | 106 | n/a | | 200 | 157 | YES | 0.51 | 1 |
| 11742 | H3 12 | 107 | n/a | | 201 | 157 | YES | 0.58 | 1 |
| 11743 | H3 13 | 108 | n/a | | 202 | 157 | YES | 0.57 | 2 |
| 11744 | L1 01 | n/a | 24 | | 156 | 203 | YES | 0.72 | 2 |
| 11745 | L1 02 | n/a | 25 | | 156 | 204 | YES | 1.12 | 1 |
| 11746 | L1 03 | n/a | 26 | | 156 | 205 | YES | 1.36 | 2 |
| 11747 | L1 04 | n/a | 27 | | 156 | 206 | YES | 0.40 | 2 |
| 11748 | L1 05 | n/a | 28 | | 156 | 207 | YES | 0.34 | 2 |
| 11749 | L1 06 | n/a | 29 | | 156 | 208 | YES | 0.98 | 3 |
| 11750 | L1 07 | n/a | 30 | | 156 | 209 | YES | 0.33 | 2 |
| 11751 | L1 08 | n/a | 31 | | 156 | 210 | YES | 0.70 | 3 |
| 11752 | L1 09 | n/a | 32 | | 156 | 211 | YES | 1.54 | 1 |
| 11753 | L1 10 | n/a | 33 | | 156 | 212 | YES | 0.40 | 2 |
| 11754 | L1 11 | n/a | 34 | | 156 | 213 | YES | 0.51 | 1 |
| 11755 | L1 12 | n/a | 35 | | 156 | 214 | YES | 0.73 | 2 |
| 11756 | L1 13 | n/a | 36 | | 156 | 215 | YES | 0.73 | 1 |
| 11757 | L1 14 | n/a | 37 | | 156 | 216 | YES | 0.09 | 3 |
| 11758 | L1 15 | n/a | 38 | A | 156 | 217 | YES | 0.33 | 1 |
| 11759 | L2 01 | n/a | 54 | | 156 | 218 | YES | 0.43 | 1 |
| 11760 | L2 02 | n/a | 55 | | 156 | 219 | YES | 0.98 | 2 |
| 11761 | L2 03 | n/a | 56 | | 156 | 220 | YES | 0.48 | 1 |
| 11762 | L2 04 | n/a | 57 | | 156 | 221 | YES | 0.67 | 1 |
| 11763 | L2 05 | n/a | 58 | | 156 | 222 | YES | 0.31 | 1 |
| 11764 | L2 06 | n/a | 59 | | 156 | 223 | YES | 0.34 | 1 |
| 11765 | L2 07 | n/a | 60 | | 156 | 224 | YES | 0.55 | 1 |
| 11766 | L3 01 | n/a | 93 | | 156 | 225 | YES | 0.35 | 1 |
| 11767 | L3 02 | n/a | 94 | A | 156 | 226 | YES | 0.38 | 1 |
| 11768 | L3 03 | n/a | 95 | | 156 | 227 | YES | 0.24 | 3 |
| 11769 | L3 04 | n/a | 96 | | 156 | 228 | YES | 0.38 | 1 |
| 11770 | L3 05 | n/a | 97 | | 156 | 229 | YES | 0.29 | 1 |
| 11771 | L3 06 | n/a | 98 | | 156 | 230 | YES | 0.54 | 3 |
| 11772 | L3 07 | n/a | 99 | | 156 | 231 | YES | 0.9 | 1 |
| 11773 | L3 08 | n/a | 100 | | 156 | 232 | YES | 0.31 | 1 |
| 11774 | L3 09 | n/a | 101 | | 156 | 233 | YES | 0.47 | 1 |

### Example 12. Construction and Screening of pH-Engineered TROP2 ABPCs

The methods of Examples 9-11 were then applied to produce double and triple substitution HCVD and LCVD variants of the constructs recited in Tables 7, 8, and 9. Higher performing HCVD and LCVD variants of Tables 7-12 were then selected to produce HCVD and LCVD) pairing variants (e.g., a substituted HCVD combined with a substituted LCVD).

TABLE 10
Sacituzumab double and triple substitution variants information, including sequence,
protein, and binding data

| | HCV | LCV | A Sub | HCV | LCV | SDS | Octet | Octet |
|---|---|---|---|---|---|---|---|---|
| MYT | Sub | Sub | Pos if | SEQ | SEQ | PAGE | loading | Binding |
| # | Positions | Positions | made | ID NO | ID NO | result | result | Result |
| 11566 | n/a | n/a | | 4 | 5 | Yes | 0.13 | n/a |
| 12294 | 32, 33 | n/a | | 313 | 5 | Yes | 0.04 | 3 |
| 12295 | 32, 60 | n/a | | 314 | 5 | Yes | 0.08 | 2 |
| 12296 | 32, 62 | n/a | | 315 | 5 | Yes | 0.05 | 2 |
| 12297 | 32, 63 | n/a | | 316 | 5 | Yes | 0.11 | 2 |
| 12298 | 32, 64 | n/a | | 317 | 5 | Yes | 0.17 | 2 |
| 12299 | 32, 65 | n/a | | 318 | 5 | Yes | 0.17 | 1 |
| 12300 | 32, 66 | n/a | | 319 | 5 | Yes | 0.13 | 2 |
| 12301 | 33, 60 | n/a | | 320 | 5 | Yes | 0.09 | 1 |
| 12302 | 33, 62 | n/a | | 321 | 5 | Yes | 0.14 | 2 |
| 12303 | 33, 63 | n/a | | 322 | 5 | Yes | 0.15 | 1 |
| 12304 | 33, 64 | n/a | | 323 | 5 | Yes | 0.13 | 1 |
| 12305 | 33, 65 | n/a | | 324 | 5 | Yes | 0.31 | 1 |
| 12306 | 33, 66 | n/a | | 325 | 5 | Yes | 0.2 | 1 |
| 12307 | 60, 62 | n/a | | 326 | 5 | Yes | 0.14 | 2 |
| 12308 | 60, 63 | n/a | | 327 | 5 | Yes | 0.13 | 2 |
| 12309 | 60, 64 | n/a | | 328 | 5 | Yes | 0.15 | 1 |
| 12310 | 60, 65 | n/a | | 329 | 5 | Yes | 0.15 | 2 |
| 12311 | 60, 66 | n/a | | 330 | 5 | Yes | 0.11 | 2 |
| 12312 | 62, 63 | n/a | | 331 | 5 | Yes | 0.53 | 2 |
| 12313 | 62, 64 | n/a | | 332 | 5 | Yes | 0.58 | 1 |
| 12314 | 62, 65 | n/a | | 333 | 5 | Yes | 0.14 | 1 |
| 12315 | 62, 66 | n/a | | 334 | 5 | Yes | 0.06 | 1 |
| 12316 | 63, 64 | n/a | | 335 | 5 | Yes | 0.21 | 2 |
| 12317 | 63, 65 | n/a | | 336 | 5 | Yes | 0.23 | 1 |
| 12318 | 63, 66 | n/a | | 337 | 5 | Yes | 0.08 | 1 |
| 12319 | 64, 65 | n/a | | 338 | 5 | Yes | 0.12 | 2 |
| 12320 | 64, 66 | n/a | | 339 | 5 | Yes | 0.14 | 2 |
| 12321 | 65, 66 | n/a | | 340 | 5 | Yes | 0.19 | 3 |
| 12322 | 33, 60, 64 | n/a | | 341 | 5 | Yes | 0.12 | 3 |
| 12323 | 33, 60, 65 | n/a | | 342 | 5 | Yes | 0.05 | 3 |
| 12324 | 33, 60, 66 | n/a | | 343 | 5 | Yes | 0.11 | 1 |
| 12325 | 33, 64, 65 | n/a | | 344 | 5 | Yes | 0.17 | 1 |
| 12326 | 33, 64, 66 | n/a | | 345 | 5 | Yes | 0.18 | 1 |
| 12327 | 33, 65, 66 | n/a | | 346 | 5 | Yes | 0.11 | 3 |
| 12328 | 60, 64, 65 | n/a | | 347 | 5 | Yes | 0.06 | 1 |
| 12329 | 60, 64, 66 | n/a | | 348 | 5 | Yes | 0.22 | 1 |
| 12330 | 60, 65, 66 | n/a | | 349 | 5 | Yes | 0.02 | 3 |
| 12331 | 64, 65, 66 | n/a | | 350 | 5 | Yes | 0.1 | 2 |
| 12332 | n/a | 30, 33 | | 4 | 351 | Yes | 0.93 | 1 |
| 12333 | n/a | 30, 51 | | 4 | 352 | Yes | 1.1 | 1 |
| 12334 | n/a | 30, 52 | | 4 | 353 | Yes | 0.67 | 1 |
| 12335 | n/a | 30, 53 | | 4 | 354 | Yes | 0.59 | 1 |
| 12336 | n/a | 30, 89 | | 4 | 355 | Yes | 0.71 | 1 |
| 12337 | n/a | 30, 91 | 91 | 4 | 356 | Yes | 0.72 | 3 |
| 12338 | n/a | 30, 92 | | 4 | 357 | Yes | 0.42 | 3 |
| 12339 | n/a | 30, 93 | | 4 | 358 | Yes | 0.68 | 2 |
| 12340 | n/a | 33, 51 | | 4 | 359 | Yes | 0.71 | 1 |
| 12341 | n/a | 33, 52 | | 4 | 360 | Yes | 0.8 | 1 |
| 12342 | n/a | 33, 53 | | 4 | 361 | Yes | 1.03 | 1 |
| 12343 | n/a | 33, 89 | | 4 | 362 | Yes | 0.59 | 1 |
| 12344 | n/a | 33, 91 | 91 | 4 | 363 | Yes | 0.52 | 3 |
| 12345 | n/a | 33, 92 | | 4 | 364 | Yes | 0.77 | 3 |
| 12346 | n/a | 33, 93 | | 4 | 365 | Yes | 0.97 | 1 |
| 12347 | n/a | 51, 52 | | 4 | 366 | Yes | 1.04 | 1 |
| 12348 | n/a | 51, 53 | | 4 | 367 | Yes | 0.98 | 1 |
| 12349 | n/a | 51, 89 | | 4 | 368 | Yes | 0.46 | 1 |
| 12350 | n/a | 51, 91 | 91 | 4 | 369 | Yes | 0.74 | 3 |
| 12351 | n/a | 51, 92 | | 4 | 370 | Yes | 1.11 | 1 |
| 12352 | n/a | 51, 93 | | 4 | 371 | Yes | 1.06 | 1 |
| 12353 | n/a | 52, 53 | | 4 | 372 | Yes | 0.5 | 1 |
| 12354 | n/a | 52, 89 | | 4 | 373 | Yes | 0.8 | 1 |
| 12355 | n/a | 52, 91 | 91 | 4 | 374 | Yes | 0.96 | 3 |
| 12356 | n/a | 52, 92 | | 4 | 375 | Yes | 0.41 | 3 |
| 12357 | n/a | 52, 93 | | 4 | 376 | Yes | 0.62 | 1 |
| 12358 | n/a | 53, 89 | | 4 | 377 | Yes | 0.52 | 1 |
| 12359 | n/a | 53, 91 | 91 | 4 | 378 | Yes | 0.44 | 3 |
| 12360 | n/a | 53, 92 | | 4 | 379 | Yes | 0.52 | 3 |
| 12361 | n/a | 53, 93 | | 4 | 380 | Yes | 0.7 | 3 |
| 12362 | n/a | 89, 91 | 91 | 4 | 381 | Yes | 0.78 | 3 |
| 12363 | n/a | 89, 92 | | 4 | 382 | Yes | 0.71 | 3 |
| 12364 | n/a | 89, 93 | | 4 | 383 | Yes | 0.7 | 1 |
| 12365 | n/a | 91, 92 | 91 | 4 | 384 | Yes | 0.05 | 3 |
| 12366 | n/a | 91, 93 | 91 | 4 | 385 | Yes | 0.6 | 3 |
| 12367 | n/a | 92, 93 | | 4 | 386 | Yes | 0.45 | 1 |
| 12368 | n/a | 30, 51, 53 | | 4 | 387 | Yes | 0.82 | 1 |
| 12369 | n/a | 30, 51, 89 | | 4 | 388 | Yes | 0.49 | 1 |
| 12370 | n/a | 30, 51, 93 | | 4 | 389 | Yes | 0.94 | 1 |
| 12371 | n/a | 30, 53, 89 | | 4 | 390 | Yes | 0.62 | 3 |
| 12372 | n/a | 30, 53, 93 | | 4 | 391 | Yes | 0.64 | 3 |
| 12373 | n/a | 30, 89, 93 | | 4 | 392 | Yes | 0.71 | 1 |
| 12374 | n/a | 51, 53, 89 | | 4 | 393 | Yes | 0.68 | 1 |
| 12375 | n/a | 51, 53, 93 | | 4 | 394 | Yes | 1.08 | 1 |
| 12376 | n/a | 51, 89, 93 | | 4 | 395 | Yes | 0.4 | 1 |
| 12377 | n/a | 53, 89, 93 | | 4 | 396 | Yes | 0.67 | 1 |

TABLE 11
Datopotamab double and triple substitution variants information,
including sequence, protein, and binding data

| | HCV | LCV | HCV | LCV | SDS | Octet | Octet |
|---|---|---|---|---|---|---|---|
| MYT | Sub | Sub | SEQ ID | SEQ ID | PAGE | loading | Binding |
| # | Positions | Positions | NO | NO | result | result | Result |
| 11635 | n/a | n/a | 80 | 81 | Yes | 0.14 | n/a |
| 12378 | 54, 98 | n/a | 397 | 81 | Yes | 0.07 | 3 |
| 12379 | 54, 101 | n/a | 398 | 81 | Yes | 0.13 | 3 |
| 12380 | 54, 104 | n/a | 399 | 81 | Yes | 0.04 | 3 |
| 12381 | 98, 101 | n/a | 400 | 81 | Yes | 0.14 | 3 |
| 12382 | 98, 104 | n/a | 401 | 81 | Yes | 0.12 | 3 |
| 12383 | 101, 104 | n/a | 402 | 81 | Yes | 0.16 | 3 |
| 12384 | 54, 98, 101 | n/a | 403 | 81 | Yes | 0.15 | 3 |
| 12385 | 54, 98, 104 | n/a | 404 | 81 | Yes | 0.09 | 3 |
| 12386 | 54, 101, 104 | n/a | 405 | 81 | Yes | 0.12 | 3 |
| 12387 | 98, 101, 104 | n/a | 406 | 81 | Yes | 0.19 | 3 |
| 12388 | n/a | 28, 51 | 80 | 407 | Yes | 0.91 | 2 |
| 12389 | n/a | 28, 52 | 80 | 408 | Yes | 0.2 | 3 |
| 12390 | n/a | 28, 89 | 80 | 409 | Yes | 0.19 | 3 |
| 12391 | n/a | 28, 92 | 80 | 410 | Yes | 0.4 | 3 |
| 12392 | n/a | 51, 52 | 80 | 411 | Yes | 0.47 | 1 |
| 12393 | n/a | 51, 89 | 80 | 412 | Yes | 0.92 | 1 |
| 12394 | n/a | 51, 92 | 80 | 413 | Yes | 0.77 | 3 |
| 12395 | n/a | 52, 89 | 80 | 414 | Yes | 0.56 | 3 |
| 12396 | n/a | 52, 92 | 80 | 415 | Yes | 0.48 | 3 |
| 12397 | n/a | 89, 92 | 80 | 416 | Yes | 0.59 | 3 |
| 12398 | n/a | 28, 51, 52 | 80 | 417 | Yes | 0.56 | 1 |
| 12399 | n/a | 28, 51, 89 | 80 | 418 | Yes | 0.83 | 1 |
| 12400 | n/a | 28, 51, 92 | 80 | 419 | Yes | 0.85 | 3 |
| 12401 | n/a | 28, 52, 89 | 80 | 420 | Yes | 0.77 | 3 |
| 12402 | n/a | 28, 52, 92 | 80 | 421 | Yes | 0.44 | 3 |
| 12403 | n/a | 28, 89, 92 | 80 | 422 | Yes | 0.6 | 3 |
| 12404 | n/a | 51, 52, 89 | 80 | 423 | Yes | 1.14 | 1 |
| 12405 | n/a | 51, 52, 92 | 80 | 424 | Yes | 0.93 | 3 |
| 12406 | n/a | 51, 89, 92 | 80 | 425 | Yes | 1 | 3 |
| 12407 | n/a | 52, 89, 92 | 80 | 426 | Yes | 0.5 | 3 |

TABLE 12
hu7E6SVG double and triple substitution variants information,
including sequence, protein, and binding data

| | HCV | LCV | Ala | HCV | LCV | SDS | Octet | Octet |
|---|---|---|---|---|---|---|---|---|
| MYT | Sub | Sub | Sub | SEQ | SEQ | PAGE | Loading | Binding |
| # | Positions | Positions | Pos | ID NO | ID NO | Result | Result | Result |
| 11704 | n/a | n/a | | 156 | 157 | Yes | 0.41 | n/a |
| 12408 | 33, 34 | n/a | | 427 | 157 | Yes | 0.68 | 1 |
| 12409 | 33, 51 | n/a | | 428 | 157 | Yes | 0.62 | 1 |
| 12410 | 33, 56 | n/a | | 429 | 157 | Yes | 0.64 | 1 |
| 12411 | 33, 102 | n/a | | 430 | 157 | Yes | 0.65 | 1 |
| 12412 | 33, 103 | n/a | | 431 | 157 | Yes | 0.59 | 1 |
| 12413 | 33, 106 | n/a | | 432 | 157 | Yes | 0.64 | 3 |
| 12414 | 34, 51 | n/a | | 433 | 157 | Yes | 0.68 | 1 |
| 12415 | 34, 56 | n/a | | 434 | 157 | Yes | 0.82 | 1 |
| 12416 | 34, 102 | n/a | | 435 | 157 | Yes | 0.85 | 1 |
| 12417 | 34, 103 | n/a | | 436 | 157 | Yes | 0.72 | 1 |
| 12418 | 34, 106 | n/a | | 437 | 157 | Yes | 0.8 | 1 |
| 12419 | 51, 56 | n/a | | 438 | 157 | Yes | 0.96 | 1 |
| 12420 | 51, 102 | n/a | | 439 | 157 | Yes | 0.67 | 1 |
| 12421 | 51, 103 | n/a | | 440 | 157 | Yes | 0.57 | 1 |
| 12422 | 51, 106 | n/a | | 441 | 157 | Yes | 0.7 | 1 |
| 12423 | 56, 102 | n/a | | 442 | 157 | Yes | 1.1 | 2 |
| 12424 | 56, 103 | n/a | | 443 | 157 | Yes | 0.73 | 1 |
| 12425 | 56, 106 | n/a | | 444 | 157 | Yes | 0.67 | 1 |
| 12426 | 102, 103 | n/a | | 445 | 157 | Yes | 0.85 | 1 |
| 12427 | 102, 106 | n/a | | 446 | 157 | Yes | 0.68 | 1 |
| 12428 | 103, 106 | n/a | | 447 | 157 | Yes | 0.45 | 1 |
| 12429 | 33, 51, 56 | n/a | | 448 | 157 | Yes | 0.82 | 2 |
| 12430 | 33, 51, 103 | n/a | | 449 | 157 | Yes | 0.77 | 1 |
| 12431 | 33, 51, 106 | n/a | | 450 | 157 | Yes | 0.79 | 3 |
| 12432 | 33, 56, 103 | n/a | | 451 | 157 | Yes | 0.84 | 1 |
| 12433 | 33, 56, 106 | n/a | | 452 | 157 | Yes | 0.77 | 3 |
| 12434 | 33, 103, 106 | n/a | | 453 | 157 | Yes | 0.65 | 3 |
| 12435 | 51, 56, 103 | n/a | | 454 | 157 | Yes | 0.91 | 1 |
| 12436 | 51, 56, 106 | n/a | | 455 | 157 | Yes | 0.75 | 2 |
| 12437 | 51, 103, 106 | n/a | | 456 | 157 | Yes | 0.59 | 1 |
| 12438 | 56, 103, 106 | n/a | | 457 | 157 | Yes | 0.72 | 1 |
| 12439 | n/a | 59, 60 | | 156 | 458 | No | −0.05 | |
| 12440 | n/a | 59, 93 | | 156 | 459 | Yes | 0.9 | 1 |
| 12441 | n/a | 59, 94 | 94 | 156 | 460 | Yes | 0.81 | 1 |
| 12442 | n/a | 59, 97 | | 156 | 461 | Yes | 0.86 | 2 |
| 12443 | n/a | 59, 101 | | 156 | 462 | Yes | 0.43 | 1 |
| 12444 | n/a | 60, 93 | | 156 | 463 | Ye | 0.91 | 2 |
| 12445 | n/a | 60, 94 | 94 | 156 | 464 | Yes | 0.82 | 1 |
| 12446 | n/a | 60, 97 | | 156 | 465 | Yes | 0.85 | 1 |
| 12447 | n/a | 60, 101 | | 156 | 466 | Yes | 0.52 | 1 |
| 12448 | n/a | 93, 94 | 94 | 156 | 467 | Yes | 0.8 | 1 |
| 12449 | n/a | 93, 97 | | 156 | 468 | Yes | 0.64 | 1 |
| 12450 | n/a | 93, 101 | | 156 | 469 | Yes | 0.58 | 1 |
| 12451 | n/a | 94, 97 | 94 | 156 | 470 | Yes | 0.62 | 1 |
| 12452 | n/a | 94, 101 | 94 | 156 | 471 | Ye | 0.77 | 2 |
| 12453 | n/a | 97, 101 | | 156 | 472 | Yes | 0.79 | 2 |
| 12454 | n/a | 59, 60, 93 | | 156 | 473 | Yes | 0.77 | 1 |
| 12455 | n/a | 59, 60, 94 | 94 | 156 | 474 | Yes | 0.79 | 1 |
| 12456 | n/a | 59, 60, 97 | | 156 | 475 | Ye | 0.89 | 3 |
| 12457 | n/a | 59, 60, 101 | | 156 | 476 | Yes | 0.85 | 3 |
| 12458 | n/a | 59, 93, 94 | 94 | 156 | 477 | Yes | 0.97 | 3 |
| 12459 | n/a | 59, 93, 97 | | 156 | 478 | Yes | | |
| 12460 | n/a | 59, 93, 101 | | 156 | 479 | Yes | 0.79 | 3 |
| 12461 | n/a | 59, 94, 97 | 94 | 156 | 480 | Yes | 0.87 | 3 |
| 12462 | n/a | 59, 94, 101 | 94 | 156 | 481 | Yes | 0.9 | 3 |
| 12463 | n/a | 59, 97, 101 | | 156 | 482 | Yes | | |
| 12464 | n/a | 60, 93, 94 | 94 | 156 | 483 | Yes | 0.81 | 3 |
| 12465 | n/a | 60, 93, 97 | | 156 | 484 | Yes | 0.85 | 3 |
| 12466 | n/a | 60, 93, 101 | | 156 | 485 | Yes | 0.65 | 1 |
| 12467 | n/a | 60, 94, 97 | 94 | 156 | 486 | Yes | | |
| 12468 | n/a | 60, 94, 101 | 94 | 156 | 487 | Yes | 0.81 | 3 |
| 12469 | n/a | 60, 97, 101 | | 156 | 488 | Yes | 0.8 | 1 |
| 12470 | n/a | 93, 94, 97 | 94 | 156 | 489 | Yes | 0.6 | 1 |
| 12471 | n/a | 93, 94, 101 | 94 | 156 | 490 | Yes | | |
| 12472 | n/a | 93, 97, 101 | | 156 | 491 | Yes | 0.64 | 1 |
| 12473 | n/a | 94, 97, 101 | 94 | 156 | 492 | Yes | 0.8 | 3 |

TABLE 13
Summary of sacituzumab heavy chain and light chain pairing variants

| | HCV | LCV | A Sub | HCV | LCV | SDS | Octet | Octet |
|---|---|---|---|---|---|---|---|---|
| MYT | Sub | Sub | Pos if | SEQ | SEQ | PAGE | loading | Binding |
| # | Positions | Positions | made | ID NO | ID NO | result | result (nm) | Result |
| 11566 | n/a | n/a | | 4 | 5 | Yes | 1.64 | n/a |
| 13160 | 32 | 30 | | 18 | 59 | Yes | 1.22 | 1 |
| 13161 | 33 | 30 | | 19 | 59 | Yes | 1.10 | 1 |
| 13162 | 60 | 30 | | 32 | 59 | Yes | 1.01 | 1 |
| 13163 | 62 | 30 | | 34 | 59 | Yes | 1.20 | 1 |
| 13164 | 63 | 30 | | 35 | 59 | Yes | 1.16 | 1 |
| 13165 | 64 | 30 | | 36 | 59 | Yes | 0.92 | 1 |
| 13166 | 65 | 30 | | 37 | 59 | Yes | 1.10 | 1 |
| 13167 | 66 | 30 | | 38 | 59 | Yes | 1.25 | 1 |
| 13168 | 32 | 33 | | 18 | 62 | Yes | 1.17 | 1 |
| 13169 | 33 | 33 | | 19 | 62 | Yes | 0.96 | 1 |
| 13170 | 60 | 33 | | 32 | 62 | Yes | 0.97 | 2 |
| 13171 | 62 | 33 | | 34 | 62 | Yes | 1.07 | 2 |
| 13172 | 63 | 33 | | 35 | 62 | Yes | 0.92 | 2 |
| 13173 | 64 | 33 | | 36 | 62 | Yes | 0.85 | 2 |
| 13174 | 65 | 33 | | 37 | 62 | Yes | 0.89 | 2 |
| 13175 | 66 | 33 | | 38 | 62 | Yes | 1.06 | 1 |
| 13176 | 32 | 51 | | 18 | 65 | Yes | 1.05 | 2 |
| 13177 | 33 | 51 | | 19 | 65 | Yes | 0.84 | 1 |
| 13178 | 60 | 51 | | 32 | 65 | Yes | 0.89 | 2 |
| 13179 | 62 | 51 | | 34 | 65 | Yes | 1.03 | 2 |
| 13180 | 63 | 51 | | 35 | 65 | Yes | 1.20 | 2 |
| 13181 | 64 | 51 | | 36 | 65 | Yes | 1.02 | 2 |
| 13182 | 65 | 51 | | 37 | 65 | Yes | 1.18 | 2 |
| 13183 | 66 | 51 | | 38 | 65 | Yes | 1.24 | 2 |
| 13184 | 32 | 52 | | 18 | 66 | Yes | 1.04 | 1 |
| 13185 | 33 | 52 | | 19 | 66 | Yes | 1.03 | 1 |
| 13186 | 60 | 52 | | 32 | 66 | Yes | 1.73 | 2 |
| 13187 | 62 | 52 | | 34 | 66 | Yes | 0.11 | 2 |
| 13188 | 63 | 52 | | 35 | 66 | Yes | 0.98 | 2 |
| 13189 | 64 | 52 | | 36 | 66 | Yes | 0.87 | 2 |
| 13190 | 65 | 52 | | 37 | 66 | Yes | 0.05 | 3 |
| 13191 | 66 | 52 | | 38 | 66 | Yes | 0.12 | 3 |
| 13192 | 32 | 53 | | 18 | 67 | Yes | 0.09 | 3 |
| 13193 | 33 | 53 | | 19 | 67 | Yes | 0.06 | 3 |
| 13194 | 60 | 53 | | 32 | 67 | Yes | 0.02 | 3 |
| 13195 | 62 | 53 | | 34 | 67 | Yes | 0.10 | 3 |
| 13196 | 63 | 53 | | 35 | 67 | Yes | 0.06 | 3 |
| 13197 | 64 | 53 | | 36 | 67 | Yes | −0.01 | 3 |
| 13198 | 65 | 53 | | 37 | 67 | Yes | 0.05 | 2 |
| 13199 | 66 | 53 | | 38 | 67 | Yes | 0.13 | 2 |
| 13200 | 32 | 89 | | 18 | 71 | Yes | 1.08 | 2 |
| 13201 | 33 | 89 | | 19 | 71 | Yes | 0.07 | 3 |
| 13202 | 60 | 89 | | 32 | 71 | Yes | 0.04 | 3 |
| 13203 | 62 | 89 | | 34 | 71 | Yes | 0.15 | 2 |
| 13204 | 63 | 89 | | 35 | 71 | Yes | 1.00 | 1 |
| 13205 | 64 | 89 | | 36 | 71 | Yes | 0.82 | 2 |
| 13206 | 65 | 89 | | 37 | 71 | Yes | 1.07 | 2 |
| 13207 | 66 | 89 | | 38 | 71 | Yes | 1.22 | 1 |
| 13208 | 32 | 91 | A | 18 | 73 | Yes | 1.41 | 3 |
| 13209 | 33 | 91 | A | 19 | 73 | Yes | 1.19 | 3 |
| 13210 | 60 | 91 | A | 32 | 73 | Yes | 1.20 | 3 |
| 13211 | 62 | 91 | A | 34 | 73 | Yes | 1.52 | 1 |
| 13212 | 63 | 91 | A | 35 | 73 | Yes | 1.43 | 2 |
| 13213 | 64 | 91 | A | 36 | 73 | Yes | 1.26 | 2 |
| 13214 | 65 | 91 | A | 37 | 73 | Yes | 1.45 | 2 |
| 13215 | 66 | 91 | A | 38 | 73 | Yes | 1.51 | 2 |
| 13216 | 32 | 92 | | 18 | 74 | | | |
| 13217 | 33 | 92 | | 19 | 74 | | | |
| 13218 | 60 | 92 | | 32 | 74 | | | |
| 13219 | 62 | 92 | | 34 | 74 | | | |
| 13220 | 63 | 92 | | 35 | 74 | | | |
| 13221 | 64 | 92 | | 36 | 74 | | | |
| 13222 | 65 | 92 | | 37 | 74 | | | |
| 13223 | 66 | 92 | | 38 | 74 | | | |
| 13224 | 32 | 93 | | 18 | 75 | Yes | 0.60 | 2 |
| 13225 | 33 | 93 | | 19 | 75 | Yes | 0.48 | 1 |
| 13226 | 60 | 93 | | 32 | 75 | Yes | 0.44 | 2 |
| 13227 | 62 | 93 | | 34 | 75 | Yes | 0.50 | 2 |
| 13228 | 63 | 93 | | 35 | 75 | Yes | 1.54 | 1 |
| 13229 | 64 | 93 | | 36 | 75 | Yes | 1.47 | 1 |
| 13230 | 65 | 93 | | 37 | 75 | Yes | 1.50 | 1 |
| 13231 | 66 | 93 | | 38 | 75 | Yes | 1.52 | 1 |
| 13232 | 32 | 51, 52 | | 18 | 366 | Yes | 1.83 | 2 |
| 13233 | 33 | 51, 52 | | 19 | 366 | Yes | 1.79 | 1 |
| 13234 | 60 | 51, 52 | | 32 | 366 | Yes | 1.69 | 1 |
| 13235 | 62 | 51, 52 | | 34 | 366 | Yes | 1.82 | 2 |
| 13236 | 63 | 51, 52 | | 35 | 366 | Yes | 1.78 | 1 |
| 13237 | 64 | 51, 52 | | 36 | 366 | Yes | 1.81 | 1 |
| 13238 | 65 | 51, 52 | | 37 | 366 | Yes | 1.85 | 2 |
| 13239 | 66 | 51, 52 | | 38 | 366 | Yes | 1.89 | 2 |
| 13240 | 32 | 51, 89 | | 18 | 368 | Yes | 1.38 | 2 |
| 13241 | 33 | 51, 89 | | 19 | 368 | Yes | 1.13 | 3 |
| 13242 | 60 | 51, 89 | | 32 | 368 | Yes | 1.13 | 2 |
| 13243 | 62 | 51, 89 | | 34 | 368 | Yes | 1.34 | 2 |
| 13244 | 63 | 51, 89 | | 35 | 368 | Yes | 1.15 | 2 |
| 13245 | 64 | 51, 89 | | 36 | 368 | Yes | 0.99 | 2 |
| 13246 | 65 | 51, 89 | | 37 | 368 | Yes | 1.28 | 2 |
| 13247 | 66 | 51, 89 | | 38 | 368 | Yes | 0.28 | 2 |
| 13248 | 32 | 52, 93 | | 18 | 376 | Yes | 0.23 | 1 |
| 13249 | 33 | 52, 93 | | 19 | 376 | Yes | 1.25 | 3 |
| 13250 | 60 | 52, 93 | | 32 | 376 | Yes | 1.31 | 1 |
| 13251 | 62 | 52, 93 | | 34 | 376 | Yes | 1.45 | 1 |
| 13252 | 63 | 52, 93 | | 35 | 376 | Yes | 1.42 | 1 |
| 13253 | 64 | 52, 93 | | 36 | 376 | Yes | 1.12 | 1 |
| 13254 | 65 | 52, 93 | | 37 | 376 | Yes | 1.34 | 1 |
| 13255 | 66 | 52, 93 | | 38 | 376 | Yes | 1.51 | 1 |
| 13366 | 32 | 30, 33 | | 18 | 351 | Yes | 1.08 | 1 |
| 13367 | 33 | 30, 33 | | 19 | 351 | Yes | 0.68 | 1 |
| 13368 | 60 | 30, 33 | | 32 | 351 | Yes | 0.97 | 1 |
| 13369 | 62 | 30, 33 | | 34 | 351 | Yes | 0.47 | 1 |
| 13370 | 63 | 30, 33 | | 35 | 351 | Yes | 0.45 | 1 |
| 13371 | 64 | 30, 33 | | 36 | 351 | Yes | 0.31 | 1 |
| 13372 | 65 | 30, 33 | | 37 | 351 | Yes | 0.98 | 1 |
| 13373 | 66 | 30, 33 | | 38 | 351 | Yes | 1.02 | 1 |
| 13374 | 32 | 30, 51 | | 18 | 352 | Yes | 0.80 | 1 |
| 13375 | 33 | 30, 51 | | 19 | 352 | Yes | 0.50 | 1 |
| 13376 | 60 | 30, 51 | | 32 | 352 | Yes | 1.05 | 1 |
| 13377 | 62 | 30, 51 | | 34 | 352 | Yes | 0.31 | 1 |
| 13378 | 63 | 30, 51 | | 35 | 352 | Yes | 0.29 | 1 |
| 13379 | 64 | 30, 51 | | 36 | 352 | Yes | 0.19 | 1 |
| 13380 | 65 | 30, 51 | | 37 | 352 | Yes | 0.84 | 1 |
| 13381 | 66 | 30, 51 | | 38 | 352 | Yes | 0.92 | 1 |
| 13382 | 32 | 30, 52 | | 18 | 353 | Yes | 0.16 | 3 |
| 13383 | 33 | 30, 52 | | 19 | 353 | Yes | 0.07 | 3 |
| 13384 | 60 | 30, 52 | | 32 | 353 | Yes | 0.41 | 1 |
| 13385 | 62 | 30, 52 | | 34 | 353 | Yes | 0.11 | 1 |
| 13386 | 63 | 30, 52 | | 35 | 353 | Yes | 0.17 | 1 |
| 13387 | 64 | 30, 52 | | 36 | 353 | Yes | 0.01 | 3 |
| 13388 | 65 | 30, 52 | | 37 | 353 | Yes | 0.36 | 1 |
| 13389 | 66 | 30, 52 | | 38 | 353 | Yes | 0.29 | 1 |
| 13390 | 32 | 33, 53 | | 18 | 361 | Yes | 1.25 | 1 |
| 13391 | 33 | 33, 53 | | 19 | 361 | Yes | 0.99 | 1 |
| 13392 | 60 | 33, 53 | | 32 | 361 | Yes | 1.17 | 1 |
| 13393 | 62 | 33, 53 | | 34 | 361 | Yes | 1.07 | 1 |
| 13394 | 63 | 33, 53 | | 35 | 361 | Yes | 0.98 | 1 |
| 13395 | 64 | 33, 53 | | 36 | 361 | Yes | 0.55 | 1 |
| 13396 | 65 | 33, 53 | | 37 | 361 | Yes | 1.17 | 1 |
| 13397 | 66 | 33, 53 | | 38 | 361 | Yes | 1.25 | 1 |
| 13398 | 32 | 33, 93 | | 18 | 365 | Yes | 0.60 | 1 |
| 13399 | 33 | 33, 93 | | 19 | 365 | Yes | 0.32 | 3 |
| 13400 | 60 | 33, 93 | | 32 | 365 | Yes | 0.96 | 1 |
| 13401 | 62 | 33, 93 | | 34 | 365 | Yes | 0.80 | 1 |
| 13402 | 63 | 33, 93 | | 35 | 365 | Yes | 0.76 | 1 |
| 13403 | 64 | 33, 93 | | 36 | 365 | Yes | 0.37 | 1 |
| 13404 | 65 | 33, 93 | | 37 | 365 | Yes | 1.06 | 1 |
| 13405 | 66 | 33, 93 | | 38 | 365 | Yes | 0.99 | 1 |
| 13406 | 32 | 51, 52 | | 18 | 366 | Yes | 1.10 | 1 |
| 13407 | 33 | 51, 52 | | 19 | 366 | Yes | 0.78 | 1 |
| 13408 | 60 | 51, 52 | | 32 | 366 | Yes | 1.17 | 1 |
| 13409 | 62 | 51, 52 | | 34 | 366 | Yes | 0.88 | 1 |
| 13410 | 63 | 51, 52 | | 35 | 366 | Yes | 0.90 | 1 |
| 13411 | 64 | 51, 52 | | 36 | 366 | Yes | 0.35 | 1 |
| 13412 | 65 | 51, 52 | | 37 | 366 | Yes | 1.11 | 1 |
| 13413 | 66 | 51, 52 | | 38 | 366 | Yes | 1.19 | 1 |
| 13414 | 32 | 30, 51, 89 | | 18 | 388 | Yes | 0.56 | 1 |
| 13415 | 33 | 30, 51, 89 | | 19 | 388 | Yes | 0.23 | 3 |
| 13416 | 60 | 30, 51, 89 | | 32 | 388 | Yes | 0.83 | 1 |
| 13417 | 62 | 30, 51, 89 | | 34 | 388 | Yes | 0.48 | 1 |
| 13418 | 63 | 30, 51, 89 | | 35 | 388 | Yes | 0.58 | 1 |
| 13419 | 64 | 30, 51, 89 | | 36 | 388 | Yes | 0.07 | 3 |
| 13420 | 65 | 30, 51, 89 | | 37 | 388 | Yes | 0.58 | 1 |
| 13421 | 66 | 30, 51, 89 | | 38 | 388 | Yes | 0.96 | 1 |
| 13422 | 32 | 30, 51, 93 | | 18 | 389 | Yes | 1.05 | 1 |
| 13423 | 33 | 30, 51, 93 | | 19 | 389 | Yes | 0.71 | 1 |
| 13424 | 60 | 30, 51, 93 | | 32 | 389 | Yes | 1.10 | 1 |
| 13425 | 62 | 30, 51, 93 | | 34 | 389 | Yes | 0.92 | 1 |
| 13426 | 63 | 30, 51, 93 | | 35 | 389 | Yes | 0.91 | 1 |
| 13427 | 64 | 30, 51, 93 | | 36 | 389 | Yes | 0.47 | 1 |
| 13428 | 65 | 30, 51, 93 | | 37 | 389 | Yes | 1.13 | 1 |
| 13429 | 66 | 30, 51, 93 | | 38 | 389 | Yes | 1.23 | 1 |
| 13430 | 32 | 51, 53, 93 | | 18 | 394 | Yes | 1.13 | 1 |
| 13431 | 33 | 51, 53, 93 | | 19 | 394 | Yes | 0.98 | 3 |
| 13432 | 60 | 51, 53, 93 | | 32 | 394 | Yes | 1.21 | 1 |
| 13433 | 62 | 51, 53, 93 | | 34 | 394 | Yes | 1.08 | 1 |
| 13434 | 63 | 51, 53, 93 | | 35 | 394 | Yes | 1.04 | 1 |
| 13435 | 64 | 51, 53, 93 | | 36 | 394 | Yes | 0.79 | 1 |
| 13436 | 65 | 51, 53, 93 | | 37 | 394 | Yes | 1.29 | 1 |
| 13437 | 66 | 51, 53, 93 | | 38 | 394 | Yes | 1.27 | 1 |

TABLE 14
Summary of datopotamab heavy chain and light chain pairing variants

| | HCV | LCV | HCV | LCV | SDS | Octet | Octet |
|---|---|---|---|---|---|---|---|
| MYT | Sub | Sub | SEQ ID | SEQ ID | PAGE | loading | Binding |
| # | Positions | Positions | NO | NO | result | result | Result |
| 11635 | n/a | n/a | 80 | 81 | Yes | 1.78 | n/a |
| 13256 | 30 | 29 | 101 | 134 | Yes | 1.67 | 1 |
| 13257 | 98 | 29 | 116 | 134 | Yes | 1.67 | 2 |
| 13258 | 101 | 29 | 119 | 134 | Yes | 1.22 | 1 |
| 13259 | 104 | 29 | 122 | 134 | Yes | 1.30 | 1 |
| 13260 | 30 | 52 | 101 | 142 | Yes | 0.19 | 3 |
| 13261 | 98 | 52 | 116 | 142 | Yes | 0.29 | 2 |
| 13262 | 101 | 52 | 119 | 142 | Yes | 0.23 | 1 |
| 13263 | 104 | 52 | 122 | 142 | Yes | −0.03 | 3 |
| 13264 | 30 | 53 | 101 | 143 | Yes | 0.60 | 2 |
| 13265 | 98 | 53 | 116 | 143 | Yes | 0.59 | 2 |
| 13266 | 101 | 53 | 119 | 143 | Yes | 0.66 | 1 |
| 13267 | 104 | 53 | 122 | 143 | Yes | 0.66 | 1 |
| 13268 | 30 | 90 | 101 | 148 | Yes | 0.21 | 2 |
| 13269 | 98 | 90 | 116 | 148 | Yes | 0.59 | 1 |
| 13270 | 101 | 90 | 119 | 148 | Yes | 0.51 | 2 |
| 13271 | 104 | 90 | 122 | 148 | Yes | 0.44 | 2 |
| 13272 | 30 | 93 | 101 | 151 | Yes | 0.59 | 2 |
| 13273 | 98 | 93 | 116 | 151 | Yes | 0.74 | 1 |
| 13274 | 101 | 93 | 119 | 151 | Yes | 0.61 | 1 |
| 13275 | 104 | 93 | 122 | 151 | Yes | 0.55 | 1 |

TABLE 15
Summary of hu7E6SVG heavy chain and light chain pairing variants

| | HCV | LCV | A Sub | HCV | LCV | SDS | Octet | Octet |
|---|---|---|---|---|---|---|---|---|
| MYT | Sub | Sub | Pos if | SEQ | SEQ | PAGE | loading | Binding |
| # | Pos | Pos | made | ID NO | ID NO | result | result | Result |
| 11704 | n/a | n/a | | 156 | 157 | Yes | 0.67 | n/a |
| 13276 | 26 | 59 | | 175 | 223 | Yes | | |
| 13277 | 27 | 59 | | 180 | 223 | Yes | | |
| 13278 | 28 | 59 | | 196 | 223 | Yes | 1.04 | 1 |
| 13279 | 29 | 59 | | 197 | 223 | Yes | 1.10 | 1 |
| 13280 | 30 | 59 | | 200 | 223 | Yes | 0.76 | 1 |
| 13281 | 31 | 60 | | 175 | 224 | Yes | | |
| 13282 | 32 | 60 | | 180 | 224 | Yes | | |
| 13283 | 33 | 60 | | 196 | 224 | Yes | 0.85 | 1 |
| 13284 | 34 | 60 | | 197 | 224 | Yes | 0.60 | 1 |
| 13285 | 35 | 60 | A | 200 | 224 | Yes | 1.02 | 1 |
| 13286 | 50 | 93 | | 175 | 225 | Yes | | |
| 13287 | 51 | 93 | | 180 | 225 | Yes | | |
| 13288 | 52 | 93 | | 196 | 225 | Yes | 0.76 | 1 |
| 13289 | 53 | 93 | | 197 | 225 | Yes | 0.29 | 1 |
| 13290 | 54 | 93 | | 200 | 225 | Yes | 0.31 | 1 |
| 13291 | 55 | 94 | | 175 | 226 | Yes | | |
| 13292 | 56 | 94 | | 180 | 226 | Yes | | |
| 13293 | 57 | 94 | | 196 | 226 | Yes | 1.07 | 1 |
| 13294 | 58 | 94 | | 197 | 226 | Yes | 0.69 | 1 |
| 13295 | 59 | 94 | | 200 | 226 | Yes | 0.57 | 3 |
| 13296 | 60 | 97 | | 175 | 229 | Yes | | |
| 13297 | 61 | 97 | | 180 | 229 | Yes | | |
| 13298 | 62 | 97 | | 196 | 229 | Yes | 0.93 | 1 |
| 13299 | 63 | 97 | | 197 | 229 | Yes | 0.61 | 1 |
| 13300 | 64 | 97 | | 200 | 229 | Yes | 1.42 | 1 |
| 13301 | 65 | n/a | | 175 | 233 | | | |
| 13302 | 96 | n/a | | 180 | 233 | | | |
| 13303 | 97 | 101 | | 196 | 233 | Yes | 0.94 | 1 |
| 13304 | 98 | 101 | | 197 | 233 | Yes | 0.55 | 1 |
| 13305 | 99 | 101 | | 200 | 233 | Yes | 0.81 | 3 |
| 13306 | 100 | 59, 94 | | 175 | 460 | Yes | 0.60 | 3 |
| 13307 | 101 | 59, 94 | | 180 | 460 | Yes | 0.67 | 3 |
| 13308 | 102 | 59, 94 | | 196 | 460 | Yes | 0.69 | 3 |
| 13309 | 103 | 59, 94 | | 197 | 460 | Yes | 0.66 | 1 |
| 13310 | 104 | 59, 94 | | 200 | 460 | Yes | 0.37 | 3 |
| 13311 | 105 | 59, 101 | | 175 | 462 | Yes | 0.62 | 3 |
| 13312 | 106 | 59, 101 | | 180 | 462 | Yes | 0.69 | 3 |
| 13313 | 107 | 59, 101 | | 196 | 462 | Yes | 0.67 | 3 |
| 13314 | 108 | 59, 101 | | 197 | 462 | Yes | 0.65 | 1 |
| 13315 | n/a | 59, 101 | | 200 | 462 | Yes | 0.62 | 3 |
| 13438 | n/a | 59, 94 | | 175 | 460 | Yes | 0.69 | 3 |
| 13439 | n/a | 59, 94 | | 180 | 460 | Yes | 0.53 | 3 |
| 13440 | n/a | 59, 94 | | 196 | 460 | Yes | 0.68 | 3 |
| 13441 | n/a | 59, 94 | | 197 | 460 | Yes | 0.81 | 1 |
| 13442 | n/a | 59, 94 | | 200 | 460 | Yes | 0.32 | 3 |
| 13443 | n/a | 59, 101 | | 175 | 462 | Yes | 0.43 | 3 |
| 13444 | n/a | 59, 101 | | 180 | 462 | Yes | 1.03 | 3 |
| 13445 | n/a | 59, 101 | | 196 | 462 | Yes | 0.90 | 3 |
| 13446 | n/a | 59, 101 | | 197 | 462 | Yes | 0.59 | 1 |
| 13447 | n/a | 59, 101 | | 200 | 462 | Yes | 0.61 | 3 |

In some embodiments, the antigen binding protein construct (ABPC) comprises a heavy chain and a light chain comprising a heavy chain variable domains (HCVD) and a light chain variable domain (LCVD) as set forth in one of the pairs of HCV and LCV SEQ ID NOs recited in Tables 13-15. In some embodiments, the pair of HCV and LCV combine to produce an antigen binding protein exhibiting a binding result of “1” (i.e., pH-dependent binding relative to its corresponding, non-engineered parent).

In some embodiments, the ABPC comprises the same heavy chain and light chain CDRs (e.g., as determined by any suitable numbering system, e.g., Kabat, Chothia, IMGT, or combinations or variations thereof e.g., the additive Kabat-IMGT method disclosed in Example 1) of an HCVD and an LCVD having the heavy chain and light chain CDRs present in one of the pairs of HCV and LCV sequences as set forth in one of the pairs of SEQ ID NOs recited in Tables 13-15. In some particular embodiments, the ABPC comprises an HCVD and LCVD having the sequences of one of the pairs of HCV and LCV SEQ ID NOs as recited in Tables 13-15, wherein the pair is associated with an octet binding result of “1”. The binding properties of selected candidates are presented in Table 16.

TABLE 16
Summary of binding data for selected heavy chain
and light chain pairing variants, including KD
and magnitude of response as determined by Octet

| | | Octet | Octet response |
|---|---|---|---|
| | | KD | at 33.33 nM |
| MYT# | DESCRIPTION | (nM) | (Y-Axis) nm |
| 11566 | Sacituzumab WT | 0.6 | 0.1569 |
| 11614 | Sacituzumab LC variant | 0.75 | 0.1493 |
| 13160 | Sacituzumab HC-LC pairing variant | 0.009 | 0.077 |
| 13368 | Sacituzumab HC-LC pairing variant | 1.03 | 0.1399 |
| 13369 | Sacituzumab HC-LC pairing variant | 0.14 | 0.1672 |
| 13370 | Sacituzumab HC-LC pairing variant | 1.36 | 0.1555 |
| 13424 | Sacituzumab HC-LC pairing variant | 1.97 | 0.1231 |
| 13425 | Sacituzumab HC-LC pairing variant | 3.9 | 0.1351 |
| 13426 | Sacituzumab HC-LC pairing variant | 1.51 | 0.1321 |
| 13427 | Sacituzumab HC-LC pairing variant | 2.12 | 0.1343 |
| 13428 | Sacituzumab HC-LC pairing variant | 2.08 | 0.1405 |
| 13429 | Sacituzumab HC-LC pairing variant | 2.85 | 0.122 |
| 13433 | Sacituzumab HC-LC pairing variant | 4.36 | 0.1203 |
| 11635 | Datopotamab WT | 1.25 | 0.1302 |
| 11670 | Datopotamab LC variant | 10.9 | 0.0891 |
| 11690 | Datopotamab LC variant | 3.4 | 0.0911 |
| 11691 | Datopotamab LC variant | 6.03 | 0.0719 |
| 11699 | Datopotamab LC variant | 3.52 | 0.0968 |
| 13265 | Datopotamab HC-LC pairing variant | 9.5 | 0.0712 |
| 11704 | hu7E6SVG WT | 4.4 | 0.1178 |
| 12459 | hu7E6SVG LCC his scan variant | 6.02 | 0.0906 |
| 13283 | h7E6SVG HC-LC pairing variant | 5.32 | 0.1085 |
| 13290 | h7E6SVG HC-LC pairing variant | 7.45 | 0.1032 |

In addition, candidates were evaluated for their ability to bind to TROP2 on TROP2-positive NCI-H1975 cells at various pH. As indicated in Table 17, pH-engineered ABPCs showed pH-dependent binding on TROP2+ cells, whilst retaining high affinity at mildly acidic pH (e.g., comparable to the mildly acidic conditions of the tumor microenvironment).

TABLE 17
Binding of selected TROP2-binding constructs
to NCI-H1975 cells at various pH

| Clone # | IC₅₀ pH 7.4 (nM) | IC₅₀ pH 6.7 (nM) | IC₅₀ pH 5.4 (nM) |
|---|---|---|---|
| Sacituzumab | 1.05 | 0.94 | 1.15 |
| MYT11614 | 0.82 | 1.061 | ~5 |
| MYT13369 | 0.63 | 1.02 | ~25 |
| MYT13370 | 0.84 | 1.86 | ~45 |
| MYT13425 | 0.81 | 2.66 | — |
| MYT13427 | 1 | 4.39 | — |
| MYT13428 | 1.56 | 8.29 | — |
| Datopotamab | 0.51 | 0.54 | 0.48 |
| MYT11699 | 0.73 | 1.16 | 4.58 |
| MYT11670 | 1.61 | 1.42 | 1.83 |
| h7E6SVG | 0.98 | 0.97 | 1.29 |
| MYT12459 | 1.42 | 1.67 | ~30 |

### Example 13. Characterization of Cellular Internalization and Endolysosomal Delivery of pH Engineered Anti-TROP2 ABPCs

Selected anti-TROP2 pH engineered antibody variants from Examples 9-12 were analyzed for internalization and endolysosomal delivery TROP2+ NCI-H1975 cells, grown per providers instructions. Cell counts were determined using trypan blue staining and the Countess II FL Automated Cell Counter (ThermoFisher; AMQAF1000). The NCI-H1975 cells were then diluted to 75,000 cells/mL and 100 μl/well are seeded into 96-well flat bottom cell culture plates (Genesee Scientific; 25-109) for overnight incubation at 37° C. and 5% C02. The following day, anti-TROP2 pH engineered antibody variants, starting ABPC antibodies, control IgG1 isotype control (BP0297, Bioxcell), and vehicle control were diluted in native culture media, and then mixed 1:1 with a 3× molar ratio Zenon pHrodo iFL Human IgG Labeling Reagent (ThermoFisher; Z25611). The mixture was incubated for 30 minutes at room temperature, the existing media was removed from the plated cells and the mixture was added. The mixture of cells, anti-TROP2 antibody variants, and Zenon pHrodo iFL Human IgG Labeling Reagent was incubated at 37° C., 5% CO₂ for 24 hours. Following incubation, the media was removed and cells are washed with 100 μL of room temperature culture medium. Following the wash 50 μL of Trypsin solution (ThermoFisher Scientific; 12605036) was added and the plate incubated at 37° C. until most of the cells were detached, followed by addition of 50 μL of culture medium. Cells were transferred to a 96-well U-bottom plate (Genesee Scientific; 25-221) and spun down at 2000 RPM for 2 minutes. Cells were washed 2× with 200 μL of ice-cold FACS buffer (phosphate buffered saline (PBS), pH 7.4+2 mM ethylenediaminetetraacetic acid (EDTA)+2% (v/v) HI FBS) before being resuspended in 150 μL of FACS buffer. Median green fluorescence intensity was detected using an Attune NxT flow cytometer (ThermoFisher Scientific). Data was analyzed using Flowjo analysis software, pHrodo green is a pH sensitive dye that fluoresces in the low pH environment of the endosomes and lysosomes and therefore can be used to quantify antibody internalization and endolysosomal delivery. Internalization and endolysosomal delivery of anti-TROP2 starting ABPC's and variants at 10 nM or 25 nM in TROP2+ cells was measured by pHrodo green median fluorescence intensity. Several pH engineered anti-TROP2 antibody variants showed increased median fluorescence intensity relative to their corresponding starting ABPC antibodies demonstrating that increased dissociation at lower pH led to enhanced internalization and endolysosomal delivery inside cells as shown by increased fluorescence or increased fluorescence as compared to IgG1 isotype control.

Increased endolysosomal delivery was quantitated for each pH engineered anti-TROP2 antibody variant on the top of each bar as a ratio of: the variant's median fluorescence intensity minus the median fluorescence intensity of the IgG control, then all divided by the variant's corresponding starting ABPC's median fluorescence intensity minus the median fluorescence intensity of the IgG control (results presented in Table 18). For example, pH-engineered variant antibodies as disclosed herein may show increased pH-dependent binding, internalization, and/or endolysosomal delivery relative to their respective parental antibodies, as particularly as exemplified by the following:

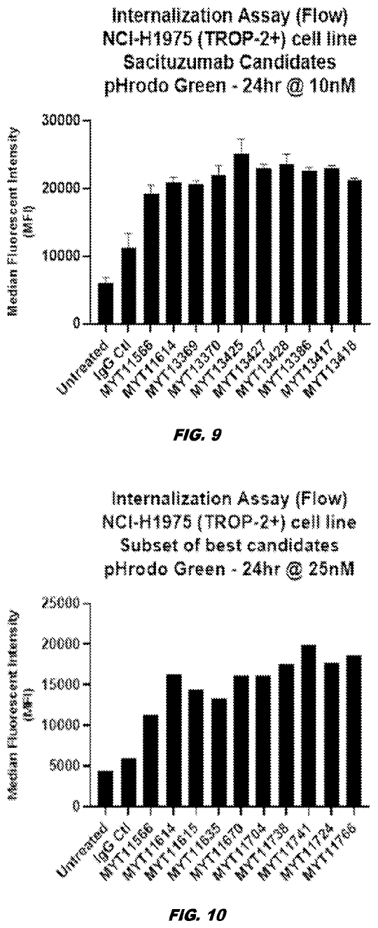

FateControl™ augmented sacituzumab constructs: MYT11573, MYT11574, MYT11577, MYT11587, MYT11589, MYT11592, MYT11593, MYT11596, MYT11605, MYT11612, MYT1613, MYT11614, MYT11615, MYT11617, MYT11620, MYT11621, MYT11622, MYT11626, MYT11628, MYT11629, MYT11630, MYT11631, MYT13369, MYT13370, MYT13425, MYT13427, MYT13428, MYT13386, MYT13417, and MYT13418 (see also FIG. 9 and FIG. 10); FateControl™ augmented datopotamab constructs: MYT11670 and MYT12459; and FateControl™ augmented hu7E6SVG constructs: MYT11724, MYT11738, MYT11741, and MYT11766.

In particular embodiments, the ABPC is selected from MYT(1614, MYT13369, MYT13370, MYT13425, MYT13427, MYT13428, MYT11635, MYT11670, MYT11699, MYT11704, and MYT12459.

Such pH engineered anti-TROP2 antibody variants with increased median fluorescence intensity relative to their starting ABPC antibodies are selected for further analysis.

TABLE 18
pH-engineered anti-TROP2 antibodies exhibiting increased
internalization in H1975 cells relative to their
respective non-engineered parent antibodies

| MYT | HCV Sub | LCV Sub | HCV SEQ | LCV SEQ | FOW H1975 |
|---|---|---|---|---|---|
| # | Position | Positions | ID NO | ID NO | (10 nM) |
| 13425 | 62 | 30, 51, 93 | 34 | 389 | 1.94 |
| 13429 | 66 | 30, 51, 93 | 38 | 389 | 1.83 |
| 13433 | 62 | 51, 53, 93 | 34 | 394 | 1.82 |
| 13417 | 62 | 30, 51, 89 | 34 | 388 | 1.76 |
| 13386 | 63 | 30, 52 | 35 | 353 | 1.71 |
| 13424 | 60 | 30, 51, 93 | 32 | 389 | 1.71 |
| 13426 | 63 | 30, 51, 93 | 35 | 389 | 1.71 |
| 13373 | 66 | 30, 33 | 38 | 351 | 1.6 |
| 13427 | 64 | 30, 51, 93 | 36 | 389 | 1.56 |
| 13428 | 65 | 30, 51, 93 | 37 | 389 | 1.55 |
| 13371 | 64 | 30, 33 | 36 | 351 | 1.51 |
| 13418 | 63 | 30, 51, 89 | 35 | 388 | 1.51 |
| 13437 | 66 | 51, 53, 93 | 38 | 394 | 1.49 |
| 13434 | 63 | 51, 53, 93 | 35 | 394 | 1.46 |
| 13369 | 62 | 30, 33 | 34 | 351 | 1.44 |
| 13368 | 60 | 30, 33 | 32 | 351 | 1.43 |
| 13370 | 63 | 30, 33 | 35 | 351 | 1.43 |
| 13376 | 60 | 30, 51 | 32 | 352 | 1.42 |
| 13421 | 66 | 30, 51, 89 | 38 | 388 | 1.42 |
| 13378 | 63 | 30, 51 | 35 | 352 | 1.4 |
| 13432 | 60 | 51, 53, 93 | 32 | 394 | 1.4 |
| 13374 | 32 | 30, 51 | 18 | 352 | 1.37 |
| 13366 | 32 | 30, 33 | 18 | 351 | 1.35 |
| 13420 | 65 | 30, 51, 89 | 37 | 388 | 1.34 |
| 13379 | 64 | 30, 51 | 36 | 352 | 1.33 |
| 13435 | 64 | 51, 53, 93 | 36 | 394 | 1.33 |
| 13395 | 64 | 33, 53 | 36 | 361 | 1.29 |
| 13416 | 60 | 30, 51, 89 | 32 | 388 | 1.29 |
| 13377 | 62 | 30, 51 | 34 | 352 | 1.26 |
| 13388 | 65 | 30, 52 | 37 | 353 | 1.24 |
| 13392 | 60 | 33, 53 | 32 | 361 | 1.19 |
| 13372 | 65 | 30, 33 | 37 | 351 | 1.18 |
| 13401 | 62 | 33, 93 | 34 | 365 | 1.16 |
| 13405 | 66 | 33, 93 | 38 | 365 | 1.16 |
| 13400 | 60 | 33, 93 | 32 | 365 | 1.13 |
| 13403 | 64 | 33, 93 | 36 | 365 | 1.13 |
| 13422 | 32 | 30, 51, 93 | 18 | 389 | 1.13 |
| 13402 | 63 | 33, 93 | 35 | 365 | 1.1 |
| 13404 | 65 | 33, 93 | 37 | 365 | 1.08 |
| 13398 | 32 | 33, 93 | 18 | 365 | 1.07 |
| 13413 | 66 | 51, 52 | 38 | 366 | 1.06 |
| 13290 | 54 | 93 | 200 | 225 | 1.7 |
| 13285 | 35 | 60 | 200 | 224 | 1.61 |
| 13298 | 62 | 97 | 196 | 229 | 1.59 |
| 13283 | 33 | 60 | 196 | 224 | 1.51 |
| 13300 | 64 | 97 | 200 | 229 | 1.39 |
| 13279 | 29 | 59 | 197 | 223 | 1.37 |
| 13289 | 53 | 93 | 197 | 225 | 1.37 |
| 13299 | 63 | 97 | 197 | 229 | 1.35 |
| 13288 | 52 | 93 | 196 | 225 | 1.15 |
| 13446 | n/a | 59, 101 | 197 | 462 | 1.09 |

### Examples of Superior Sacituzumab Variant Internalizers

As used herein (unless otherwise indicated by the context), the “same HCDRs” and “same LCDRs” means the applicable substituted or wildtype HCDRs and LCDRs present in the referenced heavy chain variable domain (HCVD) or light chain variable domain (LCVD) sequence. For example, the wildtype sacituzumab HCDRs and LCDRs may be as set forth in SEQ ID NOs: 6, 7, and 8 (HCDRs) and SEQ ID NOs: 9, 10, and 11 (LCDRs), respectively. In another example, the wildtype datopotamab HCDRs and LCDRs may be as set forth in SEQ ID NOs: 82, 83, and 84 (HCDRs) and SEQ ID NOs: 85, 86, and 87 (LCDRs), respectively. In another example, the wildtype hu7E6SVG HCDRs and LCDRs may be as set forth in SEQ ID) NOs: 158, 159, and 160 (HCDRs) and SEQ ID NOs: 161, 162, and 163 (LCDRs), respectively.

In an embodiment, the TROP2-binding protein construct comprises an HCVD set forth in SEQ ID NO: 34 and an LCVD set forth in SEQ ID NO: 389 (e.g., MYT13425). In another embodiment, the HCVD and LCVD have the same HCDRs and LCDRs as, and are at least 90%, 95%, or 98% identical to, the sequences set forth in SEQ ID NOs: 34 and 389, respectively.

In an embodiment, the TROP2-binding protein construct comprises an HCVD set forth in SEQ ID NO: 34 and an LCVD set forth in SEQ ID NO: 389 (e.g., MYT13425). In another embodiment, the HCVD and LCVD have the same HCDRs and LCDRs as, and are at least 90%, 95%, or 98% identical to, the sequences set forth in SEQ ID NOs: 34 and 389, respectively.

In an embodiment, the TROP2-binding protein construct comprises an HCVD set forth in SEQ ID NO: 38 and an LCVD set forth in SEQ ID NO: 389 (e.g., MYT13429). In another embodiment, the HCVD and LCVD have the same HCDRs and LCDRs as, and are at least 90%, 95%, or 98% identical to, the sequences set forth in SEQ ID NOs: 38 and 389, respectively.

In an embodiment, the TROP2-binding protein construct comprises an HCVD set forth in SEQ ID NO: 34 and an LCVD set forth in SEQ ID NO: 394 (e.g., MYT13433). In another embodiment, the HCVD and LCVD have the same HCDRs and LCDRs as, and are at least 90%, 95%, or 98% identical to, the sequences set forth in SEQ ID NOs: 34 and 394, respectively.

In an embodiment, the TROP2-binding protein construct comprises an HCVD set forth in SEQ ID NO: 34 and an LCVD set forth in SEQ ID NO: 388 (e.g., MYT13417). In another embodiment, the HCVD and LCVD have the same HCDRs and LCDRs as, and are at least 90%, 95%, or 98% identical to, the sequences set forth in SEQ ID NOs: 34 and 388, respectively.

In an embodiment, the TROP2-binding protein construct comprises an HCVD set forth in SEQ ID NO: 35 and an LCVD set forth in SEQ ID NO: 353 (e.g., MYT13386). In another embodiment, the HCVD and LCVD have the same HCDRs and LCDRs as, and are at least 90%, 95%, or 98% identical to, the sequences set forth in SEQ ID NOs: 35 and 353, respectively.

In an embodiment, the TROP2-binding protein construct comprises an HCVD set forth in SEQ ID NO: 32 and an LCVD set forth in SEQ ID NO: 389 (e.g., MYT13424). In another embodiment, the HCVD and LCVD have the same HCDRs and LCDRs as, and are at least 90%, 95%, or 98% identical to, the sequences set forth in SEQ ID NOs: 32 and 389, respectively.

In an embodiment, the TROP2-binding protein construct comprises an HCVD set forth in SEQ ID NO: 35 and an LCVD set forth in SEQ ID NO: 389 (e.g., MYT13426). In another embodiment, the HCVD and LCVD have the same HCDRs and LCDRs as, and are at least 90%, 95%, or 98% identical to, the sequences set forth in SEQ ID NOs: 35 and 389, respectively.

In an embodiment, the TROP2-binding protein construct comprises an HCVD set forth in SEQ ID NO: 38 and an LCVD set forth in SEQ ID NO: 351 (e.g., MYT13373). In another embodiment, the HCVD and LCVD have the same HCDRs and LCDRs as, and are at least 90%, 95%, or 98% identical to, the sequences set forth in SEQ ID NOs: 38 and 351, respectively.

In an embodiment, the TROP2-binding protein construct comprises an HCVD set forth in SEQ ID NO: 36 and an LCVD set forth in SEQ ID NO: 389 (e.g., MYT13427). In another embodiment, the HCVD and LCVD have the same HCDRs and LCDRs as, and are at least 90%, 95%, or 98% identical to, the sequences set forth in SEQ ID NOs: 36 and 389, respectively.

In an embodiment, the TROP2-binding protein construct comprises an HCVD set forth in SEQ ID NO: 37 and an LCVD set forth in SEQ ID NO: 389 (e.g., MYT13428). In another embodiment, the HCVD and LCVD have the same HCDRs and LCDRs as, and are at least 90%, 95%, or 98% identical to, the sequences set forth in SEQ ID NOs: 37 and 389, respectively.

In an embodiment, the TROP2-binding protein construct comprises an HCVD set forth in SEQ ID NO. 36 and an LCVD set forth in SEQ ID NO: 351 (e.g., MYT13371). In another embodiment, the HCVD and LCVD have the same HCDRs and LCDRs as, and are at least 90%, 95%, or 98% identical to, the sequences set forth in SEQ ID NOs: 36 and 351, respectively.

In an embodiment, the TROP2-binding protein construct comprises an HCVD set forth in SEQ ID NO: 35 and an LCVD set forth in SEQ ID NO: 388 (e.g., MYT13418). In another embodiment, the HCVD and LCVD have the same HCDRs and LCDRs as, and are at least 90%, 95%, or 98% identical to, the sequences set forth in SEQ ID NOs: 35 and 388, respectively.

In an embodiment, the TROP2-binding protein construct comprises an HCVD set forth in SEQ ID NO: 38 and an LCVD set forth in SEQ ID NO: 394 (e.g., MYT13437). In another embodiment, the HCVD and LCVD have the same HCDRs and LCDRs as, and are at least 90%, 95%, or 98% identical to, the sequences set forth in SEQ ID NOs: 38 and 394, respectively.

In an embodiment, the TROP2-binding protein construct comprises an HCVD set forth in SEQ ID NO: 35 and an LCVD set forth in SEQ ID NO: 394 (e.g., MYT13434). In another embodiment, the HCVD and LCVD have the same HCDRs and LCDRs as, and are at least 90%, 95%, or 98% identical to, the sequences set forth in SEQ ID NOs: 35 and 394, respectively.

In an embodiment, the TROP2-binding protein construct comprises an HCVD set forth in SEQ ID NO: 34 and an LCVD set forth in SEQ ID NO: 351 (e.g., MYT13369). In another embodiment, the HCVD and LCVD have the same HCDRs and LCDRs as, and are at least 90%, 95%, or 98% identical to, the sequences set forth in SEQ ID NOs: 34 and 351, respectively.

In an embodiment, the TROP2-binding protein construct comprises an HCVD set forth in SEQ ID NO: 32 and an LCVD set forth in SEQ ID NO: 351 (e.g., MYT13368). In another embodiment, the HCVD and LCVD have the same HCDRs and LCDRs as, and are at least 90%, 95%, or 98% identical to, the sequences set forth in SEQ ID NOs: 32 and 351, respectively.

In an embodiment, the TROP2-binding protein construct comprises an HCVD set forth in SEQ ID NO: 35 and an LCVD set forth in SEQ ID NO: 351 (e.g., MYT13370). In another embodiment, the HCVD and LCVD have the same HCDRs and LCDRs as, and are at least 90%, 95%, or 98% identical to, the sequences set forth in SEQ ID NOs: 35 and 351, respectively.

In an embodiment, the TROP2-binding protein construct comprises an HCVD set forth in SEQ ID NO: 32 and an LCVD set forth in SEQ ID NO: 352 (e.g., MYT13376). In another embodiment, the HCVD and LCVD have the same HCDRs and LCDRs as, and are at least 90%, 95%, or 98% identical to, the sequences set forth in SEQ ID NOs: 32 and 352, respectively.

In an embodiment, the TROP2-binding protein construct comprises an HCVD set forth in SEQ ID NO: 38 and an LCVD set forth in SEQ ID NO: 388 (e.g., MYT13421). In another embodiment, the HCVD and LCVD have the same HCDRs and LCDRs as, and are at least 90%, 95%, or 98% identical to, the sequences set forth in SEQ ID NOs: 38 and 388, respectively.

In an embodiment, the TROP2-binding protein construct comprises an HCVD set forth in SEQ ID NO: 35 and an LCVD set forth in SEQ ID NO: 352 (e.g., MYT13378). In another embodiment, the HCVD and LCVD have the same HCDRs and LCDRs as, and are at least 90%, 95%, or 98% identical to, the sequences set forth in SEQ ID NOs: 35 and 352, respectively.

In an embodiment, the TROP2-binding protein construct comprises an HCVD set forth in SEQ ID NO: 32 and an LCVD set forth in SEQ ID NO: 394 (e.g., MYT13432). In another embodiment, the HCVD and LCVD have the same HCDRs and LCDRs as, and are at least 90%, 95%, or 98% identical to, the sequences set forth in SEQ ID NOs: 32 and 394, respectively.

In an embodiment, the TROP2-binding protein construct comprises an HCVD set forth in SEQ ID NO: 18 and an LCVD set forth in SEQ ID NO: 352 (e.g., MYT13374). In another embodiment, the HCVD and LCVD have the same HCDRs and LCDRs as, and are at least 90%, 95%, or 98% identical to, the sequences set forth in SEQ ID NOs: 18 and 352, respectively.

In an embodiment, the TROP2-binding protein construct comprises an HCVD set forth in SEQ ID NO: 18 and an LCVD set forth in SEQ ID NO: 351 (e.g., MYT13366). In another embodiment, the HCVD and LCVD have the same HCDRs and LCDRs as, and are at least 90%, 95%, or 98% identical to, the sequences set forth in SEQ ID NOs: 18 and 351, respectively.

In an embodiment, the TROP2-binding protein construct comprises an HCVD set forth in SEQ ID NO: 37 and an LCVD set forth in SEQ ID NO: 388 (e.g., MYT13420). In another embodiment, the HCVD and LCVD have the same HCDRs and LCDRs as, and are at least 90%, 95%, or 98% identical to, the sequences set forth in SEQ ID NOs: 37 and 388, respectively.

In an embodiment, the TROP2-binding protein construct comprises an HCVD set forth in SEQ ID NO: 36 and an LCVD set forth in SEQ ID NO: 352 (e.g., MYT13379). In another embodiment, the HCVD and LCVD have the same HCDRs and LCDRs as, and are at least 90%, 95%, or 98% identical to, the sequences set forth in SEQ ID NOs: 36 and 352, respectively.

In an embodiment, the TROP2-binding protein construct comprises an HCVD set forth in SEQ ID NO. 36 and an LCVD set forth in SEQ ID NO: 394 (e.g., MYT13435). In another embodiment, the HCVD and LCVD have the same HCDRs and LCDRs as, and are at least 90%, 95%, or 98% identical to, the sequences set forth in SEQ ID NOs: 36 and 394, respectively.

In an embodiment, the TROP2-binding protein construct comprises an HCVD set forth in SEQ ID NO: 36 and an LCVD set forth in SEQ ID NO: 361 (e.g., MYT13395). In another embodiment, the HCVD and LCVD have the same HCDRs and LCDRs as, and are at least 90%, 95%, or 98% identical to, the sequences set forth in SEQ ID NOs: 36 and 361, respectively.

In an embodiment, the TROP2-binding protein construct comprises an HCVD set forth in SEQ ID NO: 32 and an LCVD set forth in SEQ ID NO: 388 (e.g., MYT13416). In another embodiment, the HCVD and LCVD have the same HCDRs and LCDRs as, and are at least 90%, 95%, or 98% identical to, the sequences set forth in SEQ ID NOs: 32 and 388, respectively.

In an embodiment, the TROP2-binding protein construct comprises an HCVD set forth in SEQ ID NO: 34 and an LCVD set forth in SEQ ID NO: 352 (e.g., MYT13377). In another embodiment, the HCVD and LCVD have the same HCDRs and LCDRs as, and are at least 90%, 95%, or 98% identical to, the sequences set forth in SEQ ID NOs: 34 and 352, respectively.

In an embodiment, the TROP2-binding protein construct comprises an HCVD set forth in SEQ ID NO: 37 and an LCVD set forth in SEQ ID NO: 353 (e.g., MYT13388). In another embodiment, the HCVD and LCVD have the same HCDRs and LCDRs as, and are at least 90%, 95%, or 98% identical to, the sequences set forth in SEQ ID NOs: 37 and 353, respectively.

In an embodiment, the TROP2-binding protein construct comprises an HCVD set forth in SEQ ID NO: 32 and an LCVD set forth in SEQ ID NO: 361 (e.g., MYT13392). In another embodiment, the HCVD and LCVD have the same HCDRs and LCDRs as, and are at least 90%, 95%, or 98% identical to, the sequences set forth in SEQ ID NOs: 32 and 361, respectively.

In an embodiment, the TROP2-binding protein construct comprises an HCVD set forth in SEQ ID NO: 37 and an LCVD set forth in SEQ ID NO: 351 (e.g., MYT13372). In another embodiment, the HCVD and LCVD have the same HCDRs and LCDRs as, and are at least 90%, 95%, or 98% identical to, the sequences set forth in SEQ ID NOs: 37 and 351, respectively.

In an embodiment, the TROP2-binding protein construct comprises an HCVD set forth in SEQ ID NO: 34 and an LCVD set forth in SEQ ID NO: 365 (e.g., MYT13401). In another embodiment, the HCVD and LCVD have the same HCDRs and LCDRs as, and are at least 90%, 95%, or 98% identical to, the sequences set forth in SEQ ID NOs: 34 and 365, respectively.

In an embodiment, the TROP2-binding protein construct comprises an HCVD set forth in SEQ ID NO: 38 and an LCVD set forth in SEQ ID NO: 365 (e.g., MYT13405). In another embodiment, the HCVD and LCVD have the same HCDRs and LCDRs as, and are at least 90%, 95%, or 98% identical to, the sequences set forth in SEQ ID NOs: 38 and 365, respectively.

In an embodiment, the TROP2-binding protein construct comprises an HCVD set forth in SEQ ID NO: 32 and an LCVD set forth in SEQ ID NO: 365 (e.g., MYT13400). In another embodiment, the HCVD and LCVD have the same HCDRs and LCDRs as, and are at least 90%, 95%, or 98% identical to, the sequences set forth in SEQ ID NOs: 32 and 365, respectively.

In an embodiment, the TROP2-binding protein construct comprises an HCVD set forth in SEQ ID NO: 36 and an LCVD set forth in SEQ ID NO: 365 (e.g., MYT13403). In another embodiment, the HCVD and LCVD have the same HCDRs and LCDRs as, and are at least 90%, 95%, or 98% identical to, the sequences set forth in SEQ ID NOs: 36 and 365, respectively.

In an embodiment, the TROP2-binding protein construct comprises an HCVD set forth in SEQ ID NO: 18 and an LCVD set forth in SEQ ID NO: 389 (e.g., MYT13422). In another embodiment, the HCVD and LCVD have the same HCDRs and LCDRs as, and are at least 90%, 95%, or 98% identical to, the sequences set forth in SEQ ID NOs: 18 and 389, respectively.

In an embodiment, the TROP2-binding protein construct comprises an HCVD set forth in SEQ ID NO: 35 and an LCVD set forth in SEQ ID NO: 365 (e.g., MYT13402). In another embodiment, the HCVD and LCVD have the same HCDRs and LCDRs as, and are at least 90%, 95%, or 98% identical to, the sequences set forth in SEQ ID NOs: 35 and 365, respectively.

In an embodiment, the TROP2-binding protein construct comprises an HCVD set forth in SEQ ID NO: 37 and an LCVD set forth in SEQ ID NO: 365 (e.g., MYT13404). In another embodiment, the HCVD and LCVD have the same HCDRs and LCDRs as, and are at least 90%, 95%, or 98% identical to, the sequences set forth in SEQ ID NOs: 37 and 365, respectively.

In an embodiment, the TROP2-binding protein construct comprises an HCVD set forth in SEQ ID NO: 18 and an LCVD set forth in SEQ ID NO: 365 (e.g., MYT13398). In another embodiment, the HCVD and LCVD have the same HCDRs and LCDRs as, and are at least 90%, 95%, or 98% identical to, the sequences set forth in SEQ ID NOs: 18 and 365, respectively.

In an embodiment, the TROP2-binding protein construct comprises an HCVD set forth in SEQ ID NO. 38 and an LCVD set forth in SEQ ID NO: 366 (e.g., MYT13413). In another embodiment, the HCVD and LCVD have the same HCDRs and LCDRs as, and are at least 90%, 95%, or 98% identical to, the sequences set forth in SEQ ID NOs: 38 and 366, respectively.

For each of the foregoing embodiments, the CDRs may be determined using the Kabat, IMGT, or additive Kabat-IMGT method (described in Example 1).

### Examples of Superior Hu7E6SVG Variant Internalizers.

In an embodiment, the TROP2-binding protein construct comprises an HCVD set forth in SEQ ID NO: 200 and an LCVD set forth in SEQ ID NO: 225 (e.g., MYT13290). In another embodiment, the HCVD and LCVD have the same HCDRs and LCDRs as, and are at least 90%, 95%, or 98% identical to, the sequences set forth in SEQ ID NOs: 200 and 225, respectively.

In an embodiment, the TROP2-binding protein construct comprises an HCVD set forth in SEQ ID NO: 200 and an LCVD set forth in SEQ ID NO: 224 (e.g., MYT13285). In another embodiment, the HCVD and LCVD have the same HCDRs and LCDRs as, and are at least 90%, 95%, or 98% identical to, the sequences set forth in SEQ ID NOs: 200 and 224, respectively.

In an embodiment, the TROP2-binding protein construct comprises an HCVD set forth in SEQ ID NO: 196 and an LCVD set forth in SEQ ID NO: 229 (e.g., MYT13298). In another embodiment, the HCVD and LCVD have the same HCDRs and LCDRs as, and are at least 90%, 95%, or 98% identical to, the sequences set forth in SEQ ID NOs: 196 and 229, respectively.

In an embodiment, the TROP2-binding protein construct comprises an HCVD set forth in SEQ ID NO: 196 and an LCVD set forth in SEQ ID NO: 224 (e.g., MYT13283). In another embodiment, the HCVD and LCVD have the same HCDRs and LCDRs as, and are at least 90%, 95%, or 98% identical to, the sequences set forth in SEQ ID NOs: 196 and 224, respectively.

In an embodiment, the TROP2-binding protein construct comprises an HCVD set forth in SEQ ID NO: 200 and an LCVD set forth in SEQ ID NO: 229 (e.g., MYT13300). In another embodiment, the HCVD and LCVD have the same HCDRs and LCDRs as, and are at least 90%, 95%, or 98% identical to, the sequences set forth in SEQ ID NOs: 200 and 229, respectively.

In an embodiment, the TROP2-binding protein construct comprises an HCVD set forth in SEQ ID NO: 197 and an LCVD set forth in SEQ ID NO: 223 (e.g., MYT13279). In another embodiment, the HCVD and LCVD have the same HCDRs and LCDRs as, and are at least 90%, 95%, or 98% identical to, the sequences set forth in SEQ ID NOs: 197 and 223, respectively.

In an embodiment, the TROP2-binding protein construct comprises an HCVD set forth in SEQ ID NO: 197 and an LCVD set forth in SEQ ID NO: 225 (e.g., MYT13289). In another embodiment, the HCVD and LCVD have the same HCDRs and LCDRs as, and are at least 90%, 95%, or 98% identical to, the sequences set forth in SEQ ID NOs: 197 and 225, respectively.

In an embodiment, the TROP2-binding protein construct comprises an HCVD set forth in SEQ ID NO: 197 and an LCVD set forth in SEQ ID NO: 229 (e.g., MYT13299). In another embodiment, the HCVD and LCVD have the same HCDRs and LCDRs as, and are at least 90%, 95%, or 98% identical to, the sequences set forth in SEQ ID NOs: 197 and 229, respectively.

In an embodiment, the TROP2-binding protein construct comprises an HCVD set forth in SEQ ID NO: 196 and an LCVD set forth in SEQ ID NO: 225 (e.g., MYT13288). In another embodiment, the HCVD and LCVD have the same HCDRs and LCDRs as, and are at least 90%, 95%, or 98% identical to, the sequences set forth in SEQ ID NOs: 196 and 225, respectively.

In an embodiment, the TROP2-binding protein construct comprises an HCVD set forth in SEQ ID NO: 197 and an LCVD set forth in SEQ ID NO: 462 (e.g., MYT13446). In another embodiment, the HCVD and LCVD have the same HCDRs and LCDRs as, and are at least 90%, 95%, or 98% identical to, the sequences set forth in SEQ ID NOs: 197 and 462, respectively.

For each of the foregoing embodiments, the CDRs may be determined using the Kabat, IMGT, or additive Kabat-IMGT method (described in Example 1).

### Example 14. Demonstration of Enhanced Cytotoxicity of pH-Engineered ABPC ADCs Against TROP2+ Cells

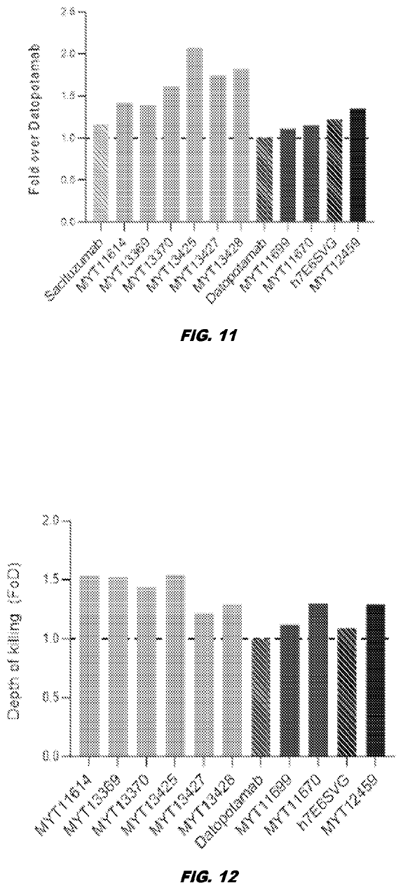

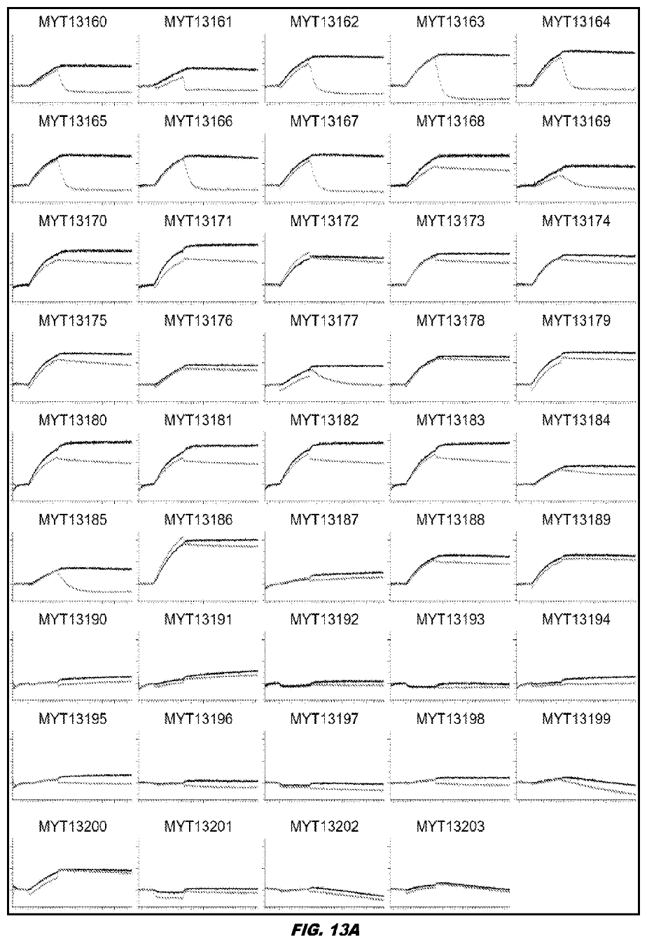

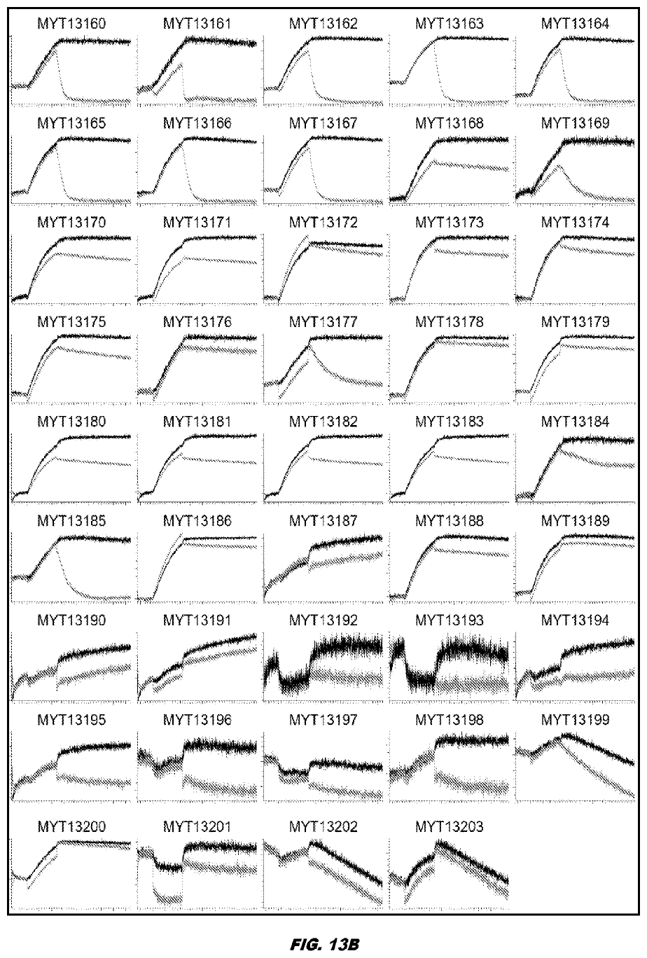

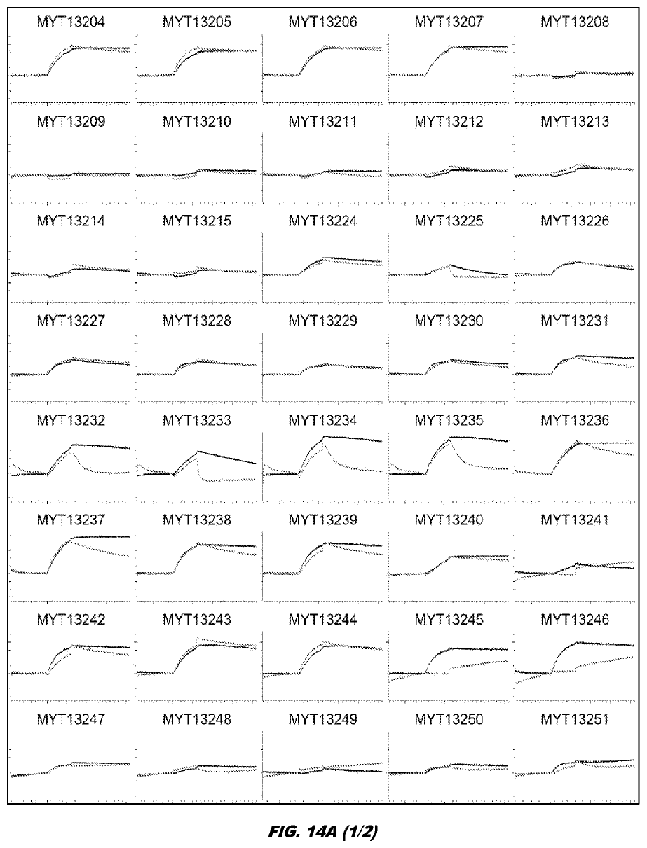

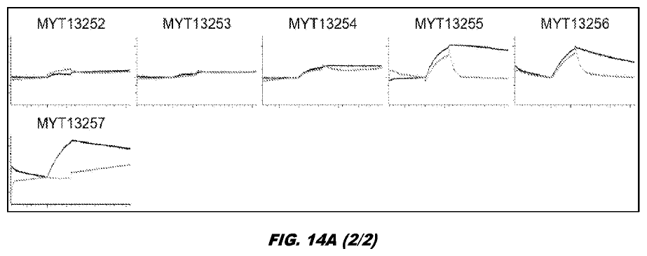

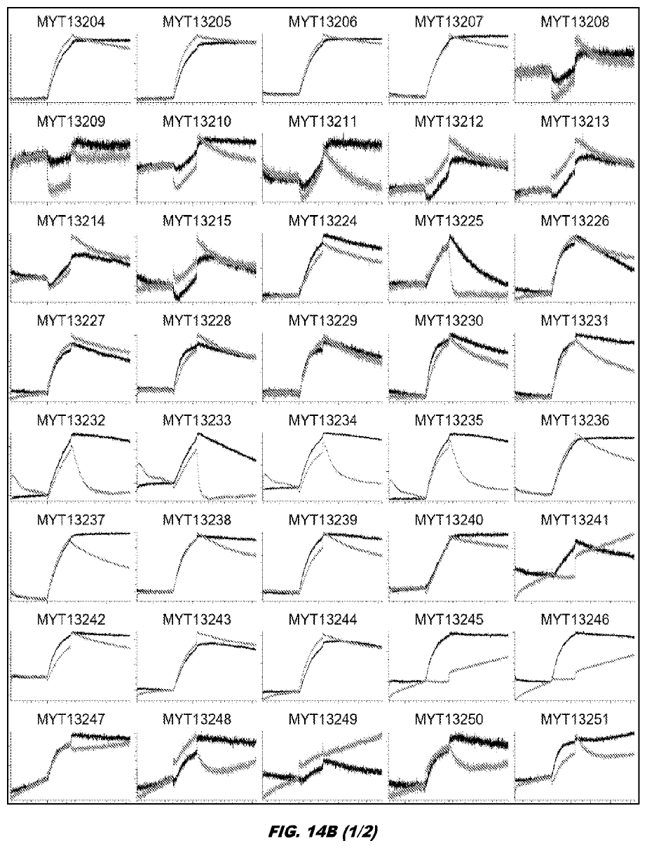

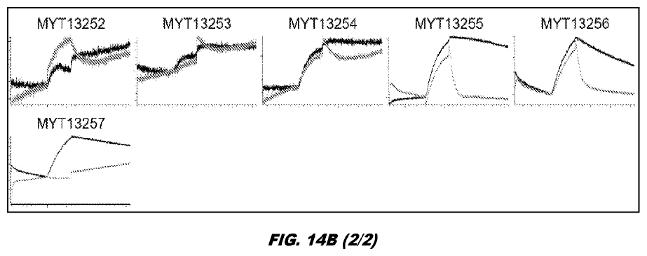

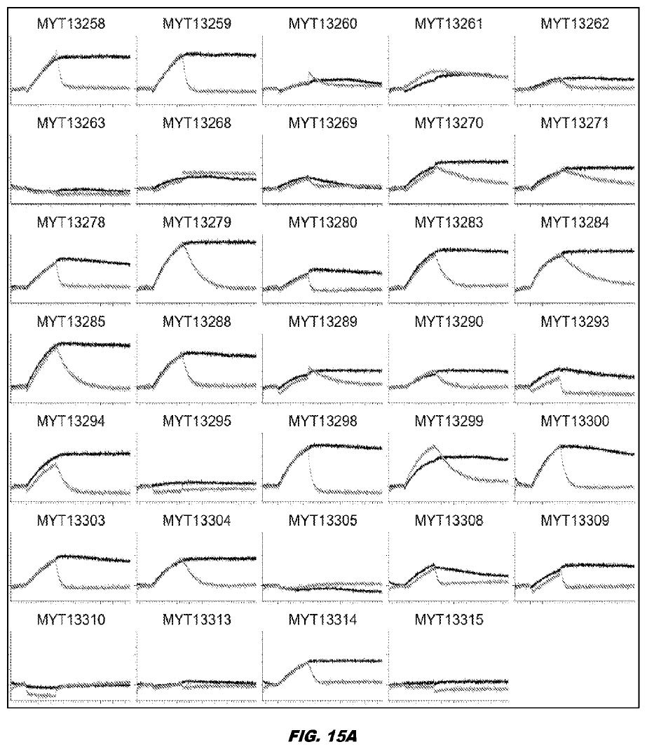

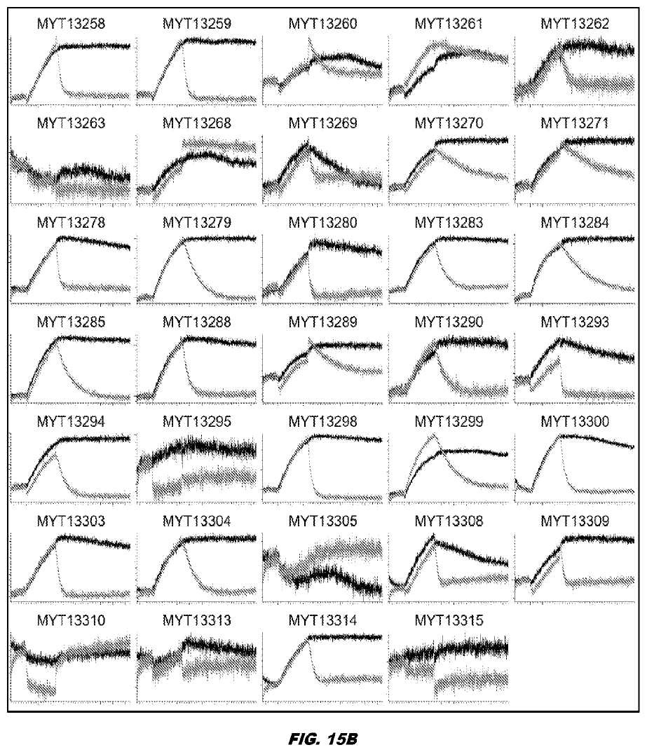

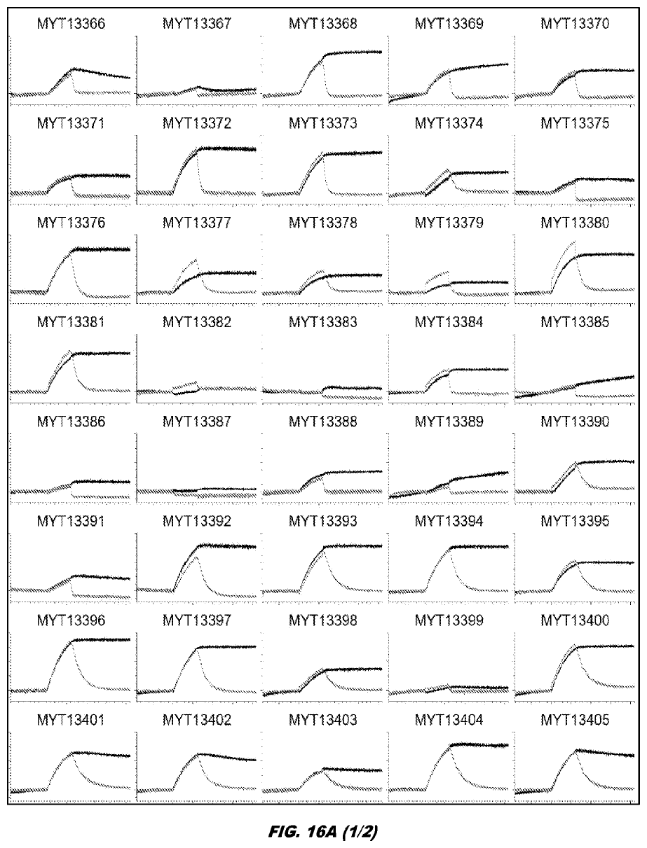

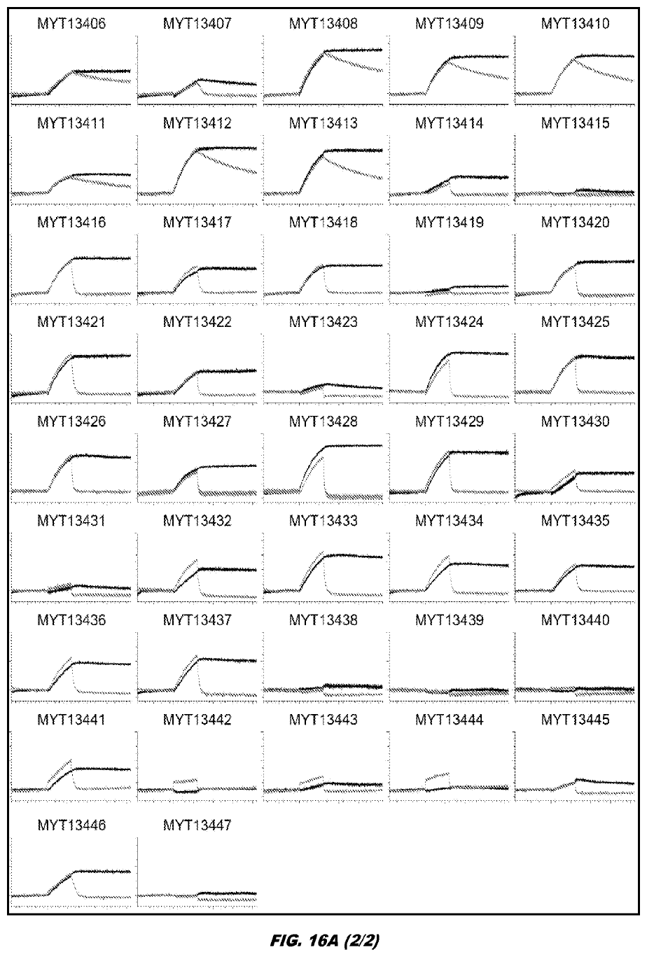

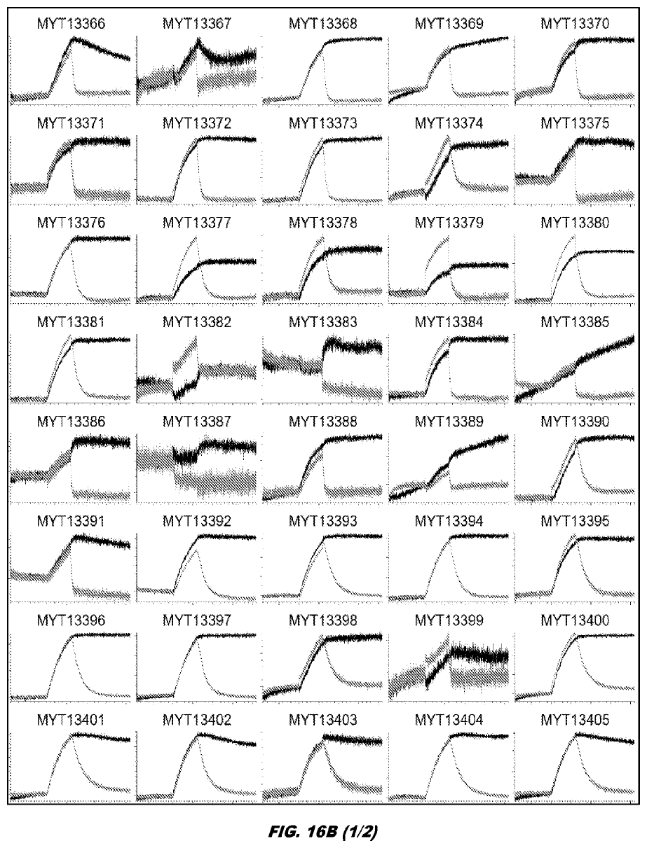

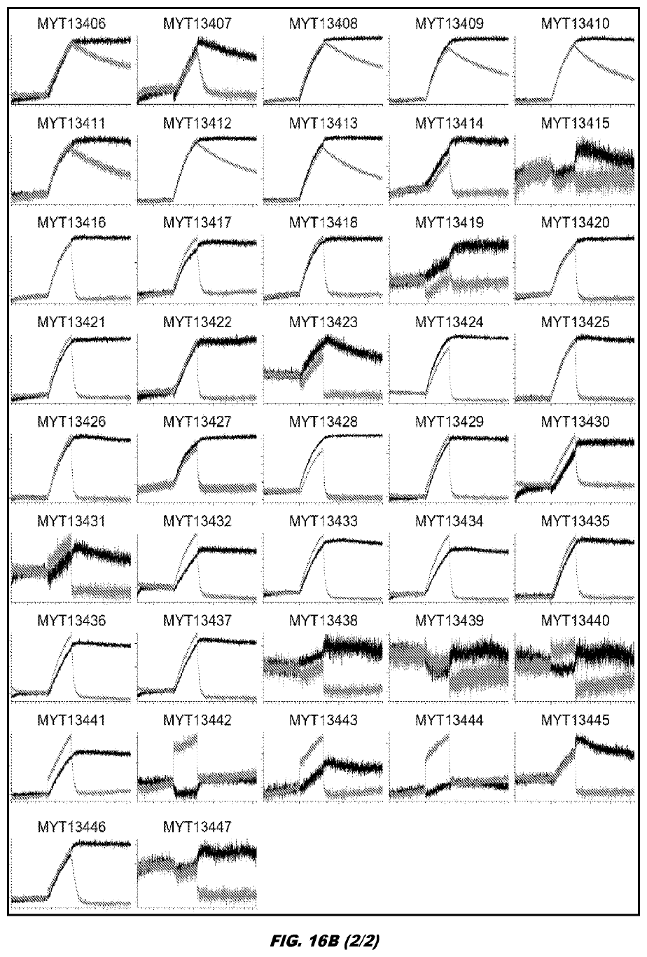

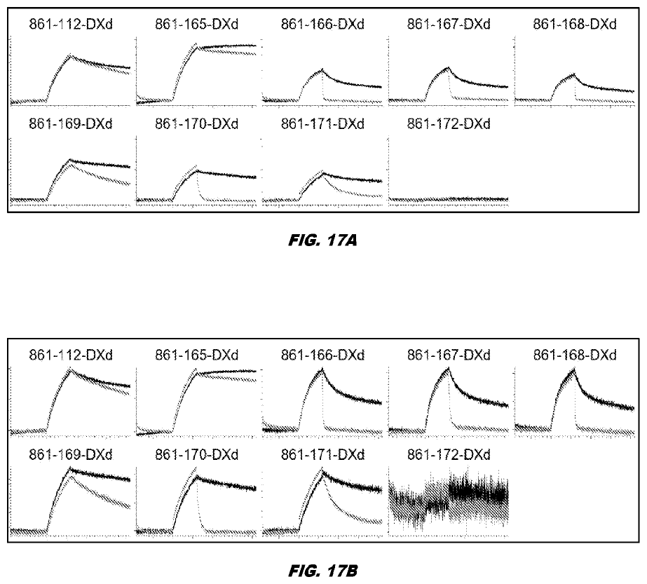

The cytotoxicity of anti-TROP-2 candidate and control antibodies was assessed by complexing them to a secondary antibody that was covalently conjugated to a non-cleavable MMAF (50 nM) and applying the complex to cells in vitro. Briefly, anti-TROP2 antibodies comprising a human IgG1 (heavy chain constant domain), a Kappa light chain constant domain, and the indicated heavy and light chain variable domains (HCVDs) were produced using techniques described herein (see Table 19). Briefly, cells were plated at about 2×10\hat{ }3 cells/well in 384 w plate and incubated overnight. Antibodies had a starting concentration of 2000 nM and were titrated 1:5 to produce an 11-point curve. 50 nM secondary-MMAF was prepared and diluted/complexed 1:1 with the antibodies. Cells were allowed to incubate for 5 days and the cell titer glow (CTG) assay was performed. The results are presented in FIGS. 11 and 12.

Particularly effective antibodies include, for example, constructs comprising the same HCV and LCV and/or the same heavy chain CDRs and light chain CDRs as the following constructs. MYT11670, MYT11614, MYT12459, MYT13160, MYT13368, MYT13369, MYT13370, MYT13371, MYT13373, MYT13424, MYT13425, MYT13426, MYT13427, MYT13428, and MYT13429.

As indicated in Table 19, there was a correlation between internalization & cytotoxicity using TROP2-positive NCI-H1975 cells. MYT12375 (one of the sacituzumab LCC variants) showed ~27% greater cytotoxicity compared to WT, non-pH-engineered datopotamab. MYT11682 (one of the datopotamab LC variants) showed ~20% greater cytotoxicity compared to WT, non-pH-engineered datopotamab. Moreover, MYT11741, MYT11766, MYT11770, and MYT12440 showed ~30% greater cytotoxicity compared to WT, non-pH-engineered datopotamab. And as across the non-augmented, WT antibodies, hu7E6SVG showed the greatest cytotoxicity. And finally, the candidates exhibited no cytotoxicity when applied to TROP2-negative NCI-H1373 cells.

TABLE 19
Binding and internalization of selected TROP2-binding constructs in NCI-H1975 cells
(formatted as heavy chain IgG1 and light chain Kappa monoclonal antibodies)

| | | | Cytotox | | Max MFI | |
|---|---|---|---|---|---|---|
| | HCV | LCV | Fold over | KD (pH | (@ | KD |
| | SEQ | SEQ | datopotamab | 7.4)- | pH 6.4)- | (OCTET) |
| MYT # | ID NO | ID NO | (FOD) | H1975 | H1975 | nM |
| MYT11566 | 4 | 5 | n/a | 1.289 | 29461 | 0.6 |
| (WT sacituzumab | | | | | | |
| variable regions) | | | | | | |
| MYT11614 | 4 | 59 | 1.35 | 0.9626 | 19414 | 0.75 |
| MYT13369 | 34 | 351 | 1.33 | 0.9194 | 17061 | 0.14 |
| MYT13370 | 35 | 351 | 1.28 | 1.58 | 13380 | 1.36 |
| MYT13425 | 34 | 389 | 1.39 | 1.354 | 11202 | 3.99 |
| MYT13427 | 36 | 389 | 1.19 | 1.443 | 6106 | 2.12 |
| MYT13428 | 37 | 389 | 1.18 | 2.543 | 6722 | 2.08 |
| MYT11635 | 80 | 81 | 1.00 | 1.283 | 25852 | 1.25 |
| (WT datopotamab | | | | | | |
| variable regions) | | | | | | |
| MYT11670 | 122 | 81 | 1.41 | 2.947 | 19955 | 10.9 |
| MYT11704 | 156 | 157 | 1.22 | 2.173 | 31485 | 4.4 |
| MYT12459 | 156 | 478 | 1.35 | 2.817 | 18732 | 6.02 |

### Example 15. Cytotoxicity of ADCs Comprising Anti-TROP2 Antibodies Hinge-Conjugated to GGFG-DXd

Antibody Production. Anti-TROP2 antibodies were produced by transient transfection of CHO cells (0.5 L scale) with the corresponding heavy chain and light chain expression plasmids. Supermatants were harvested after 7 days of incubation at 37° C. and the antibodies were purified by ProA affinity chromatography followed by size exclusion chromatography (SEC) and formulated in 20 mM His, 150 mM NaCl, pH6. Purity and identity of antibodies were assessed by SDS-PAGE, SEC-HPLC, and LC-MS. Antibodies were partially reduced for 17 h at 22° C. with TCEP and 10%?/DMA cosolvent for maleimide based GGFG-DXd conjugation to hinge cysteines with an average drug to antibody ratio (DAR) of about 4. Conjugated ADCs were purified by filtration using Amicon spin columns, DAR was assessed by hydrophobic interaction chromatography (HIC), and free drug was assessed by LC-MS. Table 20 presents a summary of the ADCs made according to the foregoing process (each antibody is formatted as a humanized monoclonal antibody comprising an IgG1 heavy chain constant domain and a Kappa light chain constant domain).

TABLE 20
Characteristics of the anti-TROP2 ADCs

| | Identifier | | | |
|---|---|---|---|---|
| | when | | HCV | LCV |
| | conjugated | | SEQ | SEQ |
| MYT # | to DXd | Description | ID NO | ID NO |
| MYT11566 | 165-DXd | Sacituzumab WT | 4 | 5 |
| MYT13425 | 166-DXd | Sacituzumab pH variant | 34 | 389 |
| MYT13370 | 167-DXd | Sacituzumab pH variant | 35 | 351 |
| MYT13427 | 168-DXd | Sacituzumab pH variant | 36 | 389 |
| MYT11704 | 169-DXd | hu7E6SVG WT | 156 | 157 |
| MYT12459 | 170-DXd | hu7E6SVG pH variant | 156 | 478 |
| MYT11670 | 171-DXd | Datopotamab pH variant | 122 | 81 |
| MYT1735 | 172-DXd | Anti-HIV gp120 antibody | | |
| | | (Non-binding control) | | |
| MYT11635 | 112-DXd | Datopotamab WT | 80 | 81 |

Cytotoxicity assays. On day 0 (DO), about 2×10\hat{ }3 TROP2+ NCI-H1975 cells were added into each well of a 384-well plate and incubated overnight at 37° C. The next day, ADC solutions were produced starting at 1000 nM and titrated 1:5 with an eleven-point curve. Five μl of the appropriate dilution was added to each well and the ADC-treated cells were incubated for five (5) days. Thereafter, the Cell Titer Glo (Promega) assay was performed substantially according to the manufacturer's protocol to evaluate the cytotoxicity of the test article ADCs relative to the non-binding control ADC.

TABLE 21
Cytotoxicity of sacituzumab pH-engineered
variants in NCI-H1975 cells

| | | % efficiency |
|---|---|---|
| Identifier | Antibody Description | @ 100 nM ADC |
| 165 DXd | Sacituzumab WT | 1.47 |
| 166 DXd | Sacituzumab variant | 9.57 |
| 167 DXd | Sacituzumab variant | 4.86 |
| 168 DXd | Sacituzumab variant | 7.32 |
| 172 DXd | Anti-HIV gp120 antibody | −0.64 |

TABLE 22
Cytotoxicity of hu7E6SVG pH-engineered
variant in NCI-H1975 cells

| | | % efficiency |
|---|---|---|
| Identifier | Antibody Description | @ 100 nM ADC |
| 169 DXd | hu7E6SVG WT | −3.50 |
| 170 DXd | hu7E6SVG variant | 5.39 |
| 112 DXd | Datopotamab WT | −1.22 |
| 172 DXd | Anti-HIV gp120 antibody | −0.64 |

Each pH-engineered ADC exhibited better cytotoxicity compared to its respective parent ADC at 100 nM.

### Example 16. Evaluation of Anti-TROP2 ABPCs In Vivo

Leading candidate anti-TROP2 ABPCs are conjugated to a cargo (e.g., an anti-cancer toxin, an immunomodulator, a nucleic acid, an inducer of protein degradation, or any other suitable cargo) and evaluated for their safety and efficacy in vivo.

In one study, conjugated candidate test articles and suitable controls are administered to mice harboring tumors from the following types of cancer cells: NCI-H1373 (TROP2-negative), NCI-H1975 (TROP2-positive), and EBC-1. Briefly, a pre-determined number of cells is injected into each mouse, tumors are permitted to form, and test articles/controls are administered to the mice when the tumors have reached about 150 to about 250 mm³. Tumor volumes are measured and timepoints post administration of the test articles/controls, and efficacy is evaluated.

In another study, about 6 mg/kg of the following test articles is administered to cynomolgus monkeys to evaluate PK and safety: non-augmented parent A, non-augmented parent B, or one of the higher performing candidate anti-TROP2 ABPC-ADCs. The cyno PK study can reveal improved recycling of pH-engineered/pH-augmented ADCs from normal tissue vs non-augmented ADC (e.g., a datopotamab-DXd comparator).

### EMBODIMENTS

Embodiment 1. A pharmaceutical composition comprising an effective amount of an antigen-binding protein construct (ABPC) comprising: a first antigen-binding domain (ABD) that is capable of specifically binding TROP2 or an epitope of TROP2 presented on the surface of a target mammalian cell, wherein: the dissociation rate of the first ABD at a pH of ~4.0-6.5 is faster than the dissociation rate at a pH of ~7.0-8.0; and/or the KD of the first ABD at a pH of ~4.0-6.5 is greater than the KD at a pH of ~7.0-8.0.

Embodiment 2. The composition of embodiment 1, wherein the first ABD comprises a heavy chain variable domain (HCVD) of sacituzumab, datopotamab, hu7E6SVG, or A1,2X4, with one or more histidine substitution, and/or wherein the HCVD comprises an H to A substitution.

Embodiment 3. The composition of embodiment 1, wherein the first ABD comprises a light chain variable domain (LCVD) of sacituzumab, datopotamab, hu7E6SVG, or A1,2X4, with one or more histidine substitution, and/or wherein the LCVD comprises an H to A substitution.

Embodiment 4. The composition of embodiment 1, wherein the ABD comprises one of (a) to (d): (a) an HCVD of sacituzumab and/or an LCVD of sacituzumab; (b) an HCVD of datopotamab and/or an LCVD of datopotamab; (c) an HCVD of hu7E6SVG; and/or an LCVD of hu7E6SVG; or (d) an HCVD of A1,2X4 and/or an LCVD of A1,2X4; wherein each HCVD and/or LCVD has one or more histidine substitution, and/or wherein each HCVD and/or LCVD comprises an H to A substitution.

Embodiment 5. The composition of embodiment 2 or 4, wherein the HCVD comprises SEQ ID NO: 4, SEQ ID NO: 80, SEQ ID NO: 156, or SEQ ID NO: 234.

Embodiment 6. The composition of embodiment 3 or 4, wherein the LCVD comprises SEQ ID NO: 5, SEQ ID NO: 81, SEQ ID NO: 157, or SEQ ID NO: 235.

Embodiment 7. The composition of embodiment 1, wherein the first ABD comprises an HCVD comprising the CDR1, 2, and 3 of one of: (a) SEQ ID NOs: 6, 7, and 8; (b) SEQ ID NOs: 82, 83, and 84; (c) SEQ ID NOs: 158, 159, and 160; and (d) SEQ ID NOs: 236, 237, and 238, wherein each set of three CDRs has collectively one or more position substituted with a histidine; and/or wherein each set of three CDRs has collectively one or more position substituted with an alanine.

Embodiment 8. The composition of embodiment 1, wherein the first ABD comprises an LCVD comprising the CDR1, 2, and 3 of one of: (a) SEQ ID NOs: 9, 10, and 11; (b) SEQ ID NOs: 85, 86, and 87; (c) SEQ ID NOs: 161, 162, and 163; and (d) SEQ ID NOs: 239, 240, and 241, wherein each set of three CDRs has collectively one or more position substituted with a histidine; and/or wherein each set of three CDRs has collectively one or more position substituted with an alanine.

Embodiment 9. The composition of embodiment 1, wherein the first ABD comprises one of (a) to (c): (a) an HCVD comprising the CDR1, 2, and 3 of SEQ ID NOs: 6, 7, and 8; and/or an LCVD comprising the CDR1, 2, and 3 of one of SEQ ID NOs: 9, 10, and 11; (b) an HCVD comprising the CDR1, 2, and 3 of one of SEQ ID NOs: 82, 83, and 84; and/or an LCVD comprising the CDR1, 2, and 3 of SEQ ID NOs: 85, 86, and 87; and (c) an HCVD comprising the CDR1, 2, and 3 of SEQ ID NOs: 158, 159, and 160; and/or an LCVD comprising the CDR1, 2, and 3 of SEQ ID NOs: 161, 162, and 163, each set of three CDRs having one or more amino acid position substituted with histidine.

Embodiment 10. The composition of embodiment 1, 2, or 7, wherein the first ABD comprises one of (a) to (c): (a) an HCVD that is at least 90% N, 95%, or 98% identical to SEQ ID NO: 4, wherein the HCVD includes a histidine at one or more positions in SEQ ID NO: 4 selected from: 29, 32, 33, 50, 52, 53, 60, 61, 62, 63, 64, 65, 66, 99, 101, 104, 105, and 108; (b) an HCVD that is at least 90%, 95%, or 98% identical to SEQ ID NO: 80, wherein the HCVD includes a histidine at one or more positions in SEQ ID NO: 80 selected from: 29, 53, 55, 64, 65, 98, 101, 104, 105, 107, and 108; and (c) an HCVD that is at least 90%, 95%, or 98% identical to SEQ ID NO: 156, wherein the HCVD includes a histidine at one or more positions in SEQ ID NO: 156 selected from: 26, 29, 30, 33, 34, 51, 54, 56, 57, 59, 102, 103, and 106.

Embodiment 11. The composition of embodiment 1, 3, or 8, wherein the first ABD comprises of one (a) to (c): (a) an LCVD that is at least 90%, 95%, or 98% identical to SEQ ID NO: 5, wherein the LCVD includes a histidine at one or more positions in SEQ ID NO: 5 selected from: 28, 29, 30, 31, 32, 33, 50, 51, 52, 53, 89, 92, 93, 94, 95, 96, and 97; (b) an LCVD that is at least 90%, 95%, or 98% identical to SEQ ID NO: 81, wherein the LCVD includes a histidine at one or more positions in SEQ ID NO: 81 selected from 26, 28, 29, 30, 31, 33, 34, 50, 51, 52, 53, 54, 89, 93, 94, and 95; and (c) an LCVD that is at least 90%, 95%, or 98% identical to SEQ ID NO: 157, wherein the LCVD includes a histidine at one or more positions in SEQ ID NO: 157 selected from 25, 32, 34, 36, 54, 56, 57, 58, 59, 60, 93, 96, 97, 99, 100, and 101.

Embodiment 12. The composition of embodiment 1, 2, or 7, wherein the first ABD comprises one of (a) to (c): (a) an HCVD that is at least 90%, 95%, or 98% identical to SEQ ID NO: 4, wherein the HCVD includes a histidine at two or more positions in SEQ ID NO: 4 selected from 29, 32, 33, 50, 52, 53, 60, 61, 62, 63, 64, 65, 66, 99, 101, 104, 105, and 108; (b) an HCVD that is at least 90%, 95%, or 98% identical to SEQ ID NO: 80, wherein the HCVD includes a histidine at two or more positions in SEQ ID NO: 80 selected from 29, 53, 55, 64, 65, 98, 101, 104, 105, 107, and 108; and (c) an HCVD that is at least 90%, 95%, or 98% identical to SEQ ID NO: 156, wherein the HCVD includes a histidine at two or more positions in SEQ ID NO: 156 selected from 26, 29, 30, 33, 34, 51, 54, 56, 57, 59, 102, 103, and 106.

Embodiment 13. The composition of embodiment 1, 3, or 8, wherein the first ABD comprises one of (a) to (c): (a) an LCVD that is at least 90%, 95%, or 98% identical to SEQ ID NO: 5, wherein the LCVD includes a histidine at two or more positions in SEQ ID NO: 5 selected from 28, 29, 30, 31, 32, 33, 50, 51, 52, 53, 89, 92, 93, 94, 95, 96, and 97; (b) an LCVD that is at least 90%, 95%, or 98% identical to SEQ ID NO: 81, wherein the LCVD includes a histidine at two or more positions in SEQ ID NO: 81 selected from 26, 28, 29, 30, 31, 33, 34, 50, 51, 52, 53, 54, 89, 93, 94, and 95; and (c) an LCVD that is at least 90%, 95%, or 98% identical to SEQ ID NO: 157, wherein the LCVD includes a histidine at two or more positions in SEQ ID NO: 157 selected from 25, 32, 34, 36, 54, 56, 57, 58, 59, 60, 93, 96, 97, 99, 100, and 101.

Embodiment 14. The composition of embodiment 1, 4, or 9, wherein the first ABD comprises one of (a) to (c): (a) an HCVD that is at least 90%, 95%, or 98% identical to SEQ ID NO: 4, wherein the HCVD includes a histidine at one or more positions in SEQ ID NO: 4 selected from 29, 32, 33, 50, 52, 53, 60, 61, 62, 63, 64, 65, 66, 99, 101, 104, 105, and 108; and/or an LCVD that is at least 90%, 95%, or 98% identical to SEQ ID NO: 5, wherein the LCVD includes a histidine at one or more positions in SEQ ID NO: 5 selected from 28, 29, 30, 31, 32, 33, 50, 51, 52, 53, 89, 92, 93, 94, 95, 96, and 97; (b) an HCVD that is at least 90%, 95%, or 98% identical to SEQ ID NO: 80, wherein the HCVD includes a histidine at one or more positions in SEQ ID NO: 80 selected from 29, 53, 55, 64, 65, 98, 101, 104, 105, 107, and 108; and/or an LCVD that is at least 90%, 95%, or 98% identical to SEQ ID NO: 81, wherein the LCVD includes a histidine at one or more positions in SEQ ID NO: 81 selected from 26, 28, 29, 30, 31, 33, 34, 50, 51, 52, 53, 54, 89, 93, 94, and 95; and (c) an HCVD that is at least 90%, 95%, or 98% identical to SEQ ID NO: 156, wherein the HCVD includes a histidine at one or more positions in SEQ ID NO: 156 selected from 26, 29, 30, 33, 34, 51, 54, 56, 57, 59, 102, 103, and 106; and/or an LCVD that is at least 90%, 95%, or 98% identical to SEQ ID NO: 157, wherein the LCVD includes a histidine at one or more positions in SEQ ID NO: 157 selected from 25, 32, 34, 36, 54, 56, 57, 58, 59, 60, 93, 96, 97, 99, 100, and 101.

Embodiment 15. The composition of embodiment 1, wherein the first ABD comprises an HCVD comprising the sequence of SEQ ID NO: 4, one of the sequences of SEQ ID NOs: 12-52, SEQ ID NO: 80, one of the sequences of SEQ ID NOs: 88-128, SEQ ID NO: 156, one of the sequences of 164-202, SEQ ID NO: 234, one of the sequences set forth in SEQ ID NOs: 242-280, or one of the sequences set forth in SEQ ID NOs: 318, 320, 322, 323, 324, 325, 328, 332, 333, 334, 336, 337, 343, 344, 345, 347, 348, 427, 428, 429, 430, 431, 433, 434, 435, 436, 437, 438, 439, 440, 441, 443, 444, 445, 446, 447, 449, 451, 454, 456, and 457.

Embodiment 16. The composition of embodiment 1 or 15, wherein the first ABD comprises an LCVD comprising the sequence of SEQ ID NO: 5, one of the sequences set forth in SEQ ID NOs: 53-79, SEQ ID NO: 81, one of the sequences set forth in SEQ ID NOs: 129-155, SEQ ID NO: 157, one of the sequences set forth in SEQ ID NOs: 203-233, SEQ ID NO: 235, one of the sequences set forth in SEQ ID NOs: 281-312, or one of the sequences set forth in SEQ ID NOs: 351, 352, 353, 354, 355, 359, 360, 361, 362, 365, 366, 367, 368, 370, 371, 372, 373, 376, 377, 383, 386, 387, 388, 389, 392, 393, 394, 395, 396, 411, 412, 417, 418, 423, 459, 460, 462, 464, 465, 466, 467, 468, 469, 470, 473, 474, 485, 488, 489, and 491.

Embodiment 17. The composition of embodiment 1, wherein the first ABD comprises one of (a) to (c): (a) an HCVD of SEQ ID NO: 4, one of the sequences set forth in SEQ ID NOs: 12-52, or one of the sequences set forth in SEQ ID NOs: 313-350, and/or an LCVD of SEQ ID NO: 5, one of the sequences set forth in SEQ ID NOs: 53-79, or one of the sequences set forth in SEQ ID NOs: 351-396, wherein the first ABD does not comprise (i) SEQ ID NOs: 4 and 5; (ii) an HCVD of SEQ ID NO: 4 and an LCVD that is not one of the sequences set forth in SEQ ID NOs: 57, 58, 59, 60, 61, 62, 64, 65, 66, 67, 71, 74, 75, 76, 77, 78, and 79; or (iii) an LCVD of SEQ ID NO: 5 and an HCVD that is not one of SEQ ID NOs: 15, 18, 19, 22, 24, 25, 32, 33, 34, 35, 36, 37, 38, 41, 43, 46, 47, 50, 318, 320, 322, 323, 324, 325, 328, 332, 333, 334, 336, 337, 343, 344, 345, 347, and 348; (b) an HCVD of SEQ ID NO: 80, one of the sequences set forth in SEQ ID NOs: 88-128, one of the sequences set forth in SEQ ID NOs: 397-406, or one of the sequences set forth in SEQ ID NOs: 397-406, and/or an LCVD of SEQ ID NO: 81, one of the sequences set forth in SEQ ID NOs: 129-155, or one of the sequences set forth in SEQ ID NOs: 407-426, wherein the first ABD does not comprise (i) SEQ ID NOs: 80 and 81; (ii) an HCVD of SEQ ID NO: 80 and an LCVD that is not one of SEQ ID NOs: 131, 133, 134, 135, 136, 138, 139, 140, 141, 142, 143, 144, 147, 151, 152, 153, 411, 412, 417, 418, and 423; or (iii) an LCVD of SEQ ID NO: 81 and an HCVD that is not one of SEQ ID NOs: 91, 101, 103, 112, 113, 116, 119, 122, 123, 125, and 126; and (c) an HCVD of SEQ ID NO: 156, one of the sequences set forth in SEQ ID NOs: 164-202, or one of the sequences set forth in SEQ ID NOs: 427-457, and/or an LCVD of SEQ ID NO: 157, one of the sequences set forth in SEQ ID NOs: 203-233, or one of the sequences set forth in SEQ ID NOs: 458-492, wherein the first ABD does not comprise (i) SEQ ID NOs: 156 and 157; (ii) an HCVD of SEQ ID NO: 156 and an LCVD that is not one of SEQ ID NOs: 204, 211, 213, 215, 218, 220, 221, 222, 223, 224, 225, 228, 229, 231, 232, 233, 459, 460, 462, 464, 465, 466, 467, 468, 469, 470, 473, 474, 485, 488, 489, and 491; or (iii) an LCVD of SEQ ID NO: 157 and an HCVD that is not one of the sequences set forth in SEQ ID NOs: 164, 167, 168, 171, 172, 175, 178, 180, 181, 183, 196, 197, 200, 201, 427, 428, 429, 430, 431, 433, 434, 435, 436, 437, 438, 439, 440, 441, 443, 444, 445, 446, 447, 449, 451, 454, 456, and 457.

Embodiment 18. The composition of any one of embodiments 1-17, wherein the ABPC is degraded in the target mammalian cell following internalization of the ABPC by the target mammalian cell.

Embodiment 19. The composition of any one of embodiments 1-18, wherein the ABPC further comprises a conjugated toxin, radioisotope, drug, or small molecule.

Embodiment 20. The composition of embodiment 19, wherein the composition provides for an increase in toxin liberation in the target mammalian cell as compared to a composition comprising the same amount of a control ABPC.

Embodiment 21. The composition of embodiment 20, wherein the composition provides for at least a 20%, 50%, 2-fold, or 5-fold increase in toxin liberation in the target mammalian cell as compared to a composition comprising the same amount of a control ABPC.

Embodiment 22. The composition of any one of embodiments 19-21, wherein the composition provides for an increase in target mammalian cell killing as compared to a composition comprising the same amount of a control ABPC.

Embodiment 23. The composition of embodiment 22, wherein the composition provides for at least a 20%, 50%, 2-fold, or 5-fold increase in target mammalian cell killing as compared to a composition comprising the same amount of a control ABPC.

Embodiment 24. The composition of any one of embodiments 1-23, wherein the composition provides for an increase in endolysosomal delivery in the target mammalian cell as compared to a composition comprising the same amount of a control ABPC.

Embodiment 25. The composition of embodiment 24, wherein the composition provides for at least a 20%, 50%, 2-fold, or 5-fold increase in endolysosomal delivery in the target mammalian cell as compared to a composition comprising the same amount of a control ABPC.

Embodiment 26. The composition of any one of embodiments 1-25, wherein the composition results in a less or no detectable reduction in the level of TROP2 presented on the surface of the target mammalian cell as compared to a composition comprising the same amount of a control ABPC.

Embodiment 27. A composition comprising an effective amount of an antigen-binding protein construct (ABPC) comprising: a first ABD that is capable of specifically binding TROP2 or an epitope of TROP2 presented on the surface of a target mammalian cell; and a conjugated toxin, radioisotope, drug, or small molecule, wherein: (a) the dissociation rate of the first ABD at a pH of ~4.0-6.5 is faster than the dissociation rate at a pH of ~7.0-8.0; or the dissociation constant (KD) of the first ABD at a pH of ~4.0-6.5 is greater than the KD at a pH of ~7.0-8.0; and (b) the composition provides for one or more of: (i) an increase in toxin liberation in the target mammalian cell as compared to a composition comprising the same amount of a control ABPC; (ii) an increase in target mammalian cell killing as compared to a composition comprising the same amount of a control ABPC; or (iii) an increase in endolysosomal delivery in the target mammalian cell as compared to a composition comprising the same amount of a control ABPC.

Embodiment 28. The composition of embodiment 27, wherein the first ABD comprises a heavy chain variable domain (HCVD) of one of (a) to (d): an HCVD of (a) sacituzumab; (b) datopotamab; (c) hu7E6SVG; and (d) A1,2X4, each with one or more amino acids substituted with a histidine.

Embodiment 29. The composition of embodiment 27, wherein the first ABD comprises a light chain variable domain (LCVD) of one of: an LCVD of (a) sacituzumab; (b) datopotamab; (c) hu7E6SVG; and (d) A1,2X4, each with one or more amino acids substituted with a histidine.

Embodiment 30. The composition of embodiment 27, wherein the ABD comprises one of (a) an HCVD of sacituzumab and/or an LCVD of sacituzumab; (b) an HCVD of datopotamab and/or an LCVD of datopotamab; (c) an HCVD of hu7E6SVG and/or an LCVD of hu7E6SVG; or (d) an HCVD of A1,2X4 and/or an LCVD of A1,2X4, wherein each HCVD or LCVD has one or more amino acid substituted with a histidine.

Embodiment 31. The composition of embodiment 28 or 30, wherein the HCVD comprises one of: (a) an HCVD of sacituzumab comprising SEQ ID NO: 4; (b) an HCVD of datopotamab comprising SEQ ID NO: 80; (c) an HCVD of hu7E6SVG comprising SEQ ID NO: 156; and (d) an HCVD of A1,2X4 comprising SEQ ID NO: 234.

Embodiment 32. The composition of embodiment 29 or 30, wherein the LCVD comprises one of: (a) an LCVD of sacituzumab comprising SEQ ID NO: 5; (b) an LCVD of datopotamab comprising SEQ ID NO: 81; (c) an LCVD of hu7E6SVG comprising SEQ ID NO: 157; and (d) an LCVD of A1,2X4 comprising SEQ ID NO: 235.

Embodiment 33. The composition of embodiment 27, wherein the first ABD comprises an HCVD comprising the CDR1, 2, and 3 of one of: (a) SEQ ID NOs: 6, 7, and 8; (b) SEQ ID NOs: 82, 83, and 84; (c) SEQ ID NOs: 158, 159, and 160; and (d) SEQ ID NOs: 236, 237, and 238, wherein each set of three CDRs has collectively one or more amino acid position substituted with a histidine.

Embodiment 34. The composition of embodiment 27, wherein the first ABD comprises an LCVD comprising the CDR1, 2, and 3 of one of: (a) SEQ ID NOs: 9, 10, and 11; (b) SEQ ID NOs: 85, 86, and 87; (c) SEQ ID NOs: 161, 162, and 163; and (d) SEQ ID NOs: 239, 240, and 241, wherein each set of three CDRs has collectively one or more amino acid position substituted with a histidine.

Embodiment 35. The composition of embodiment 27, wherein the first ABD comprises one of (a) to (c): (a) an HCVD comprising the CDR1, 2, and 3 of SEQ ID NOs: 6, 7, and 8; and/or an LCVD comprising the CDR1, 2, and 3 of one of SEQ ID NOs: 9, 10, and 11; (b) an HCVD comprising the CDR1, 2, and 3 of one of SEQ ID NOs: 82, 83, and 84; and/or an LCVD comprising the CDR1, 2, and 3 of SEQ ID NOs: 85, 86, and 87; (c) an HCVD comprising the CDR1, 2, and 3 of SEQ ID NOs: 158, 159, and 160; and/or an LCVD comprising the CDR1, 2, and 3 of SEQ ID NOs: 161, 162, and 163; each set of three HCVD and/or LCVD CDRs having one or more amino acid position substituted with histidine.

Embodiment 36. The composition of embodiment 27, 28, or 33, wherein the first ABD comprises one of (a) to (c): (a) an LCVD that is at least 90%, 95%, or 98% identical to SEQ ID NO: 5, wherein the LCVD includes a histidine at one or more positions in SEQ ID NO: 5 selected from 28, 29, 30, 31, 32, 33, 50, 51, 52, 53, 89, 92, 93, 94, 95, 96, and 97; (b) an LCVD that is at least 90%, 95%, or 98% identical to SEQ ID NO: 81, wherein the LCVD includes a histidine at one or more positions in SEQ ID NO: 81 selected from 26, 28, 29, 30, 31, 33, 34, 50, 51, 52, 53, 54, 89, 93, 94, and 95; and (c) an LCVD that is at least 90%, 95%, or 98% identical to SEQ ID NO: 157, wherein the LCVD includes a histidine at one or more positions in SEQ ID NO: 157 selected from 25, 32, 34, 36, 54, 56, 57, 58, 59, 60, 93, 96, 97, 99, 100, and 101.

Embodiment 37. The composition of embodiment 27, 29, or 34, wherein the first ABD comprises one of (a) to (c): (a) an HCVD that is at least 90%, 95%, or 98% identical to SEQ ID NO: 4, wherein the HCVD includes a histidine at two or more positions in SEQ ID NO: 4 selected from 29, 32, 33, 50, 52, 53, 60, 61, 62, 63, 64, 65, 66, 99, 101, 104, 105, and 108; (b) an HCVD that is at least 90%, 95%, or 98% identical to SEQ ID NO: 80, wherein the HCVD includes a histidine at two or more positions in SEQ ID NO: 80 selected from 29, 53, 55, 64, 65, 98, 101, 104, 105, 107, and 108; and (c) an HCVD that is at least 90%, 95%, or 98% identical to SEQ ID NO: 156, wherein the HCVD includes a histidine at two or more positions in SEQ ID NO: 156 selected from 26, 29, 30, 33, 34, 51, 54, 56, 57, 59, 102, 103, and 106.

Embodiment 38. The composition of embodiment 27, 28, or 33, wherein the first ABD comprises one of (a) to (c): (a) an LCVD that is at least 90%, 95%, or 98% identical to SEQ ID NO: 5, wherein the LCVD includes a histidine at two or more positions in SEQ ID NO: 5 selected from two of 28, 29, 30, 31, 32, 33, 50, 51, 52, 53, 89, 92, 93, 94, 95, 96, and 97; (b) an LCVD that is at least 90%, 95%, or 98% identical to SEQ ID NO: 81, wherein the LCVD includes a histidine at two or more positions in SEQ ID NO: 81 selected from 26, 28, 29, 30, 31, 33, 34, 50, 51, 52, 53, 54, 89, 93, 94, and 95; and (c) an LCVD that is at least 90%, 95%, or 98% identical to SEQ ID NO: 157, wherein the LCVD includes a histidine at two or more positions in SEQ ID NO: 157 selected from 25, 32, 34, 36, 54, 56, 57, 58, 59, 60, 93, 96, 97, 99, 100, and 101.

Embodiment 39. The composition of embodiment 27, 29, 34, wherein the first ABD comprises one of (a) to (c): (a) an HCVD that is at least 90%, 95%, or 98% identical to SEQ ID NO: 4, wherein the HCVD includes a histidine at one or more positions in SEQ ID NO: 4 selected from 29, 32, 33, 50, 52, 53, 60, 61, 62, 63, 64, 65, 66, 99, 101, 104, 105, and 108; and/or an LCVD that is at least 90%, 95%, or 98% identical to SEQ ID NO: 5, wherein the LCVD includes a histidine at one or more positions in SEQ ID NO: 5 selected from 28, 29, 30, 31, 32, 33, 50, 51, 52, 53, 89, 92, 93, 94, 95, 96, and 97; (b) an HCVD that is at least 90%, 95%, or 98% identical to SEQ ID NO: 80, wherein the HCVD includes a histidine at one or more positions in SEQ ID NO: 80 selected from 29, 53, 55, 64, 65, 98, 101, 104, 105, 107, and 108; and/or an LCVD that is at least 90%, 95%, or 98% identical to SEQ ID NO: 81, wherein the LCVD includes a histidine at one or more positions in SEQ ID NO: 81 selected from 26, 28, 29, 30, 31, 33, 34, 50, 51, 52, 53, 54, 89, 93, 94, and 95; and (c) an HCVD that is at least 90%, 95%, or 98% identical to SEQ ID NO: 156, wherein the HCVD includes a histidine at one or more positions in SEQ ID NO: 156 selected from 26, 29, 30, 33, 34, 51, 54, 56, 57, 59, 102, 103, and 106; and/or an LCVD that is at least 90%, 95%, or 98% identical to SEQ ID NO: 157, wherein the LCVD includes a histidine at one or more positions in SEQ ID NO: 157 selected from 25, 32, 34, 36, 54, 56, 57, 58, 59, 60, 93, 96, 97, 99, 100, and 101.

Embodiment 40. The composition of embodiment 27, 30, or 35, wherein the first ABD comprises an HCVD comprising SEQ ID NO: 4, one of the sequences set forth in SEQ ID NOs: 12-52, SEQ ID NO: 80, one of the sequences set forth in SEQ ID NOs: 88-128, SEQ ID NO: 156, one of the sequences set forth in SEQ ID NOs164-202, SEQ ID NO: 234, one of the sequences set forth in SEQ ID NOs: 242-280, or one of the sequences set forth in SEQ ID NOs: 318, 320, 322, 323, 324, 325, 328, 332, 333, 334, 336, 337, 343, 344, 345, 347, 348, 427, 428, 429, 430, 431, 433, 434, 435, 436, 437, 438, 439, 440, 441, 443, 444, 445, 446, 447, 449, 451, 454, 456, and 457.

Embodiment 41. The composition of embodiment 27, wherein the first ABD comprises an LCVD comprising the sequence of SEQ ID NO: 5, one of the sequences set forth in SEQ ID NOs: 53-79, SEQ ID NO: 81, one of the sequences set forth in SEQ ID NOs: 129-155, SEQ ID NO: 157, one of the sequences set forth in SEQ ID NOs: 203-233, SEQ ID NO: 235, one of the sequences set forth in SEQ ID NOs: 281-312, or one of the sequences set forth in SEQ ID NOs: 351, 352, 353, 354, 355, 359, 360, 361, 362, 365, 366, 367, 368, 370, 371, 372, 373, 376, 377, 383, 386, 387, 388, 389, 392, 393, 394, 395, 396, 411, 412, 417, 418, 423, 459, 460, 462, 464, 465, 466, 467, 468, 469, 470, 473, 474, 485, 488, 489, and 491

Embodiment 42. The composition of embodiment 27 or 41, wherein the first ABD comprises one of (a) to (c): (a) an HCVD of SEQ ID NO: 4, one of the sequences set forth in SEQ ID NOs: 12-52, or one of the sequences set forth in SEQ ID NOs: 313-350, and/or an LCVD of SEQ ID NO: 5, one of the sequences set forth in SEQ ID NOs: 53-79, or one of the sequences set forth in SEQ ID NOs: 351-396, wherein the first ABD does not comprise (i) SEQ ID NOs: 4 and 5; (ii) an HCVD of SEQ ID NO: 4 and an LCVD that is not one of the sequences set forth in SEQ ID NOs: 57, 58, 59, 60, 61, 62, 64, 65, 66, 67, 71, 74, 75, 76, 77, 78, 79, 351, 352, 353, 354, 355, 359, 360, 361, 362, 365, 366, 367, 368, 370, 371, 372, 373, 376, 377, 383, 386, 387, 388, 389, 392, 393, 394, 395, and 396; or (iii) an LCVD of SEQ ID NO: 5 and an HCVD that is not one of SEQ ID NOs: 15, 18, 19, 22, 24, 25, 32, 33, 34, 35, 36, 37, 38, 41, 43, 46, 47, 50, 318, 320, 322, 323, 324, 325, 328, 332, 333, 334, 336, 337, 343, 344, 345, 347, and 348; (b) an HCVD of SEQ ID NO: 80, one of the sequences set forth in SEQ ID NOs: 88-128, one of the sequences set forth in SEQ ID NOs: 397-406, or one of the sequences set forth in SEQ ID NOs: 397406, and/or an LCVD of SEQ ID NO: 81, one of the sequences set forth in SEQ ID NOs: 129-155, or one of the sequences set forth in SEQ ID NOs: 407-426, wherein the first ABD does not comprise (i) SEQ ID NOs: 80 and 81; (ii) an HCVD of SEQ ID NO: 80 and an LCVD that is not one of SEQ ID NOs: 131, 133, 134, 135, 136, 138, 139, 140, 141, 142, 143, 144, 147, 151, 152, 153, 411, 412, 417, 418, and 423; or (iii) an LCVD of SEQ ID NO: 81 and an HCVD that is not one of SEQ ID NOs: 91, 101, 103, 112, 113, 116, 119, 122, 123, 125, and 126; and (c) an HCVD of SEQ ID NO: 156, one of the sequences set forth in SEQ ID NOs: 164-202, or one of the sequences set forth in SEQ ID NOs: 427-457, and/or an LCVD of SEQ ID NO: 157, one of the sequences set forth in SEQ ID NOs: 203-233, or one of the sequences set forth in SEQ ID NOs: 458-492, wherein the first ABD does not comprise (i) SEQ ID NOs: 156 and 157; (ii) an HCVD of SEQ ID NO: 156 and an LCVD that is not one of SEQ ID NOs: 204, 211, 213, 215, 218, 220, 221, 222, 223, 224, 225, 228, 229, 231, 232, 233, 459, 460, 462, 464, 465, 466, 467, 468, 469, 470, 473, 474, 485, 488, 489, and 491; or (iii) an LCVD of SEQ ID NO: 157 and an HCVD that is not one of the sequences set forth in SEQ ID NOs: 164, 167, 168, 171, 172, 175, 178, 180, 181, 183, 196, 197, 200, 201, 427, 428, 429, 430, 431, 433, 434, 435, 436, 437, 438, 439, 440, 441, 443, 444, 445, 446, 447, 449, 451, 454, 456, and 457.

Embodiment 43. The composition of any one of embodiments 27-42, wherein the composition provides for at least a 20%, 50%, 2-fold, or 5-fold increase in toxin liberation in the target mammalian cell, and/or cell killing, and/or at least a 20%, 50%, 2-fold, or 5-fold increase in endolysosomal delivery as compared to a composition comprising the same amount of a control ABPC.

Embodiment 44. The composition of any one of embodiments 1-43, wherein the dissociation rate of the ABD at a pH of ~4.0-6.5 is at least 10%, 3-fold, or 10-fold faster than the dissociation rate of the ABD at a pH of ~7.0-8.0 and/or the KD of the ABD at a pH of ~4.0-6.5 is at least 10%, 3-fold, or 10-fold greater than the KD of the ABD at a pH of ~7.0-8.0.

Embodiment 45. The composition of any one of embodiments 1-44, wherein the ABPC comprises a single polypeptide, optionally wherein the ABD is selected from a VH domain, a VHH domain, a VNAR domain, and a scFv; and/or wherein the ABPC is a BiTe, a (scFv)2, a nanobody, a nanobody-HSA, a DART, a TandAb, a scDiabody, a scDiabody-CH3, scFv-CH-CL-scFv, a HSAbody, scDiabody-HSA, or a tandem-scFv.

Embodiment 46. The composition of any one of embodiments 1-45, wherein the ABPC comprises two or more polypeptides.

Embodiment 47. The composition of any one of embodiments 17-46, wherein at least one polypeptide of the ABPC is conjugated to the toxin, the radioisotope, the drug, or the small molecule via a cleavable or non-cleavable linker.

Embodiment 48. The composition of any of embodiments 1-47, wherein the half-life of the ABPC in vivo is increased as compared to the half-life of a control ABPC in vivo, optionally wherein the increase is ~5%-95%, ~10%-95%, ~30%-95%, ~50%-95%, or ~70%-95% as compared to the half-life of a control ABPC in vivo.

Embodiment 49. The composition of any one of embodiments 17-48, wherein the control ABPC is capable of specifically binding to TROP2 or an epitope of TROP2 presented on the surface of a target mammalian cell, wherein: (a) the control ABPC comprises a first ABD; (b) the dissociation rate of the first ABD of the control ABPC at a pH of 4.0-6.5 is no more than 2-fold or 3-fold faster than the dissociation rate at a pH of ~7.0-8.0; and (c) the dissociation constant (KD) of the first ABD of the control ABPC at a pH of 4.0-6.5 is no more than 2-fold or 3-fold greater than the KD at a pH of ~7.0-8.0.

Embodiment 50. The composition of any one of embodiments 20-49, wherein the control ABPC is sacituzumab, datopotamab, hu7E6SVG, or A1,2X4.

Embodiment 51. The composition of any one of embodiments 1-50, wherein the ABPC further comprises a second ABD.

Embodiment 52. A kit comprising at least one dose of the composition of any one of embodiments 1-51.

Embodiment 53. An antigen-binding protein construct (ABPC) comprising: a first antigen-binding domain (ABD) that is capable of specifically binding TROP2 or an epitope of TROP2 presented on the surface of a target mammalian cell, wherein: the dissociation rate of the first ABD at a pH of ~4.0-6.5 is faster than the dissociation rate at a pH of ~7.0-8.0; or the KD of the first ABD at a pH of ~4.0-6.5 is greater than the KD at a pH of ~7.0-8.0.

Embodiment 54. The ABPC of embodiment 53, wherein the first ABD comprises an HCVD of one of an HCVD of (a) sacituzumab; (b) datopotamab; (c) hu7E6SVG, and (d) A1,2X4 and/or an LCVD of one of an LCVD of (a) sacituzumab; (b) datopotamab; (c) hu7E6SVG; and (d) A1,2X4, each HCVD or LCVD with one or more amino acids substituted with a histidine.

Embodiment 55. The ABPC of embodiment 54, wherein the HCVD comprises one of: (a1) an HCVD of sacituzumab comprising SEQ ID NO: 4; (b1) an HCVD of datopotamab comprising SEQ ID NO: 80; (c1) an HCVD of hu7E6SVG comprising SEQ ID NO: 156; and (d1) an HCVD of A1,2X4 comprising SEQ ID NO: 234; and one of a corresponding: (a2) an LCVD of sacituzumab comprising SEQ ID NO: 5; (b2) an LCVD of datopotamab comprising SEQ ID NO: 81; (c2) an LCVD of hu7E6SVG comprising SEQ ID NO. 157, and (d2) an LCVD of A1,2X4 comprising SEQ ID NO: 235, wherein each HCVD and/or LCVD has one or more histidine substitution.

Embodiment 56. The ABPC of embodiment 55, wherein the first ABD comprises an HCVD comprising the CDR1, 2, and 3 of one of: (a1) SEQ ID NOs: 6, 7, and 8; (b1) SEQ ID NOs: 82, 83, and 84; (c1) SEQ ID NOs: 158, 159, and 160; and (d1) SEQ ID NOs: 236, 237, and 238, and a corresponding LCVD comprising the CDR1, 2, and 3 of one of a corresponding: (a2) SEQ ID NOs: 9, 10, and 11; (b2) SEQ ID NOs: 85, 86, and 87; (c2) SEQ ID NOs: 161, 162, and 163; and (d2) SEQ LD NOs: 239, 240, and 241, wherein each set of three CDRs has collectively one or more amino acid position substituted with a histidine.

Embodiment 57. The ABPC of any one of embodiments 54-56, comprising an HCVD of one of (a) to (c):
(a) an HCVD that is at least 90%, 95%, or 98% identical to SEQ ID NO: 4, wherein the HCVD includes a histidine at one or more positions in SEQ ID NO: 4 selected from 29, 32, 33, 50, 52, 53, 60, 61, 62, 63, 64, 65, 66, 99, 101, 104, 105, and 108, (b) an HCVD that is at least 90%, 95%, or 98% identical to SEQ ID NO: 80, wherein the HCVD includes a histidine at one or more positions in SEQ ID NO: 80 selected from 29, 53, 55, 64, 65, 98, 101, 104, 105, 107, and 108; and (c) an HCVD that is at least 90%, 95%, or 98% identical to SEQ ID NO: 156, wherein the HCVD includes a histidine at one or more positions in SEQ ID NO: 156 selected from 26, 29, 30, 33, 34, 51, 54, 56, 57, 59, 102, 103, and 106.

Embodiment 58. The ABPC of any one of embodiments 54-56, comprising an LCVD of one of comprises one of (a) to (d): (a) an LCVD that is at least 90%, 95%, or 98% identical to SEQ ID NO: 5, wherein the LCVD includes a histidine at one or more positions in SEQ ID NO: 5 selected from 28, 29, 30, 31, 32, 33, 50, 51, 52, 53, 89, 92, 93, 94, 95, 96, and 97; (b) an LCVD that is at least 90%, 95%, or 98% identical to SEQ ID NO: 80, wherein the LCVD includes a histidine at one or more positions in SEQ ID NO: 80 selected from 26, 28-31, 33, 34, 50-54, 89, 93, 94, and 95; (c) an LCVD that is at least 90%, 95%, or 98% identical to SEQ ID NO: 157, wherein the LCVD includes a histidine at one or more positions in SEQ ID NO: 157 selected from 25, 32, 34, 36, 54, 56, 57, 58, 59, 60, 93, 96, 97, 99, 100, and 101; and (d) an LCVD that is at least 90%, 95%, or 98% identical to SEQ ID NO: 235, wherein the LCVD includes a histidine at one or more positions in SEQ ID NO: 235.

Embodiment 59. The ABPC of 57 or 58, wherein the HCVD and/or LCVD includes a histidine at two or more positions in its defining SEQ ID NO.

Embodiment 60. The ABPC of embodiment 53, wherein the first ABD comprises an HCVD comprising the sequence of SEQ ID NO: 4, one of the sequences of SEQ ID NOs: 12-52, SEQ ID NO: 80, one of the sequences of SEQ ID NOs: 88-128, SEQ ID NO: 156, one of the sequences of 164-202, SEQ ID NO: 234, one of the sequences set forth in SEQ ID NOs: 242-280, or one of the sequences set forth in SEQ ID NOs: 318, 320, 322, 323, 324, 325, 328, 332, 333, 334, 336, 337, 343, 344, 345, 347, 348, 427, 428, 429, 430, 431, 433, 434, 435, 436, 437, 438, 439, 440, 441, 443, 444, 445, 446, 447, 449, 451, 454, 456, and 457; and/or the first ABD comprises an LCVD comprising the sequence of SEQ ID NO: 5, one of the sequences set forth in SEQ ID NOs: 53-79, SEQ ID NO: 81, one of the sequences set forth in SEQ ID NOs: 129-155, SEQ ID NO: 157, one of the sequences set forth in SEQ ID NOs: 203-233, SEQ ID NO: 235, one of the sequences set forth in SEQ ID NOs: 281-312, or one of the sequences set forth in SEQ ID NOs: 351, 352, 353, 354, 355, 359, 360, 361, 362, 365, 366, 367, 368, 370, 371, 372, 373, 376, 377, 383, 386, 387, 388, 389, 392, 393, 394, 395, 396, 411, 412, 417, 418, 423, 459, 460, 462, 464, 465, 466, 467, 468, 469, 470, 473, 474, 485, 488, 489, and 491.

Embodiment 61. The ABPC of embodiment 53, wherein the first ABD comprises one of comprises one of (a) to (c): (a) an HCVD of SEQ ID NO: 4, one of the sequences set forth in SEQ ID NOs: 12-52, or one of the sequences set forth in SEQ LD NOs: 313-350, and/or an LCVD of SEQ ID NO: 5, one of the sequences set forth in SEQ ID NOs: 53-79, or one of the sequences set forth in SEQ ID NOs: 351-396, wherein the first ABD does not comprise (i) SEQ ID NOs: 4 and 5; (ii) an HCVD of SEQ ID NO: 4 and an LCVD that is not one of the sequences set forth in SEQ ID NOs: 57, 58, 59, 60, 61, 62, 64, 65, 66, 67, 71, 74, 75, 76, 77, 78, 79, 351, 352, 353, 354, 355, 359, 360, 361, 362, 365, 366, 367, 368, 370, 371, 372, 373, 376, 377, 383, 386, 387, 388, 389, 392, 393, 394, 395, and 3%; or (iii) an LCVD of SEQ ID NO: 5 and an HCVD that is not one of SEQ ID NOs: 15, 18, 19, 22, 24, 25, 32, 33, 34, 35, 36, 37, 38, 41, 43, 46, 47, 50, 318, 320, 322, 323, 324, 325, 328, 332, 333, 334, 336, 337, 343, 344, 345, 347, and 348; (b) an HCVD of SEQ ID NO: 80, one of the sequences set forth in SEQ ID NOs: 88-128, one of the sequences set forth in SEQ ID NOs: 397-406, or one of the sequences set forth in SEQ ID NOs: 397-406, and/or an LCVD of SEQ ID NO: 81, one of the sequences set forth in SEQ ID NOs: 129-155, or one of the sequences set forth in SEQ ID NOs: 407-426, wherein the first ABD does not comprise (i) SEQ ID NOs: 80 and 81; (ii) an HCVD of SEQ ID NO: 80 and an LCVD that is not one of SEQ ID NOs: 131, 133, 134, 135, 136, 138, 139, 140, 141, 142, 143, 144, 147, 151, 152, 153, 411, 412, 417, 418, and 423; or (iii) an LCVD of SEQ ID NO: 81 and an HCVD that is not one of SEQ ID NOs: 91, 101, 103, 112, 113, 116, 119, 122, 123, 125, and 126; and (c) an HCVD of SEQ ID NO: 156, one of the sequences set forth in SEQ ID NOs: 164-202, or one of the sequences set forth in SEQ ID NOs: 427-457, and/or an LCVD of SEQ ID NO: 157, one of the sequences set forth in SEQ ID NOs: 203-233, or one of the sequences set forth in SEQ ID NOs: 458-492, wherein the first ABD does not comprise (i) SEQ ID NOs: 156 and 157; (ii) an HCVD of SEQ ID NO: 156 and an LCVD that is not one of SEQ ID NOs: 204, 211, 213, 215, 218, 220, 221, 222, 223, 224, 225, 228, 229, 231, 232, 233, 459, 460, 462, 464, 465, 466, 467, 468, 469, 470, 473, 474, 485, 488, 489, and 491; or (iii) an LCVD of SEQ ID NO: 157 and an HCVD that is not one of the sequences set forth in SEQ ID NOs: 164, 167, 168, 171, 172, 175, 178, 180, 181, 183, 196, 197, 200, 201, 427, 428, 429, 430, 431, 433, 434, 435, 436, 437, 438, 439, 440, 441, 443, 444, 445, 446, 447, 449, 451, 454, 456, and 457.

Embodiment 62. The ABPC of any one of embodiments 53-61, wherein the ABPC is degraded in the target mammalian cell following internalization of the ABPC by the cell; and/or wherein the ABPC further comprises a conjugated toxin, radioisotope, drug, or small molecule; and/or wherein the ABPC provides for an increase in toxin liberation in the cell as compared to the same amount of a control ABPC.

Embodiment 63. The ABPC of embodiment 62, wherein the ABPC provides for at least a 20%, 50%, 2-fold, or 5-fold increase in toxin liberation; and/or wherein the ABPC provides for an increase in target mammalian cell killing as compared to the same amount of a control ABPC.

Embodiment 64. The ABPC of embodiment 63, wherein the composition provides for at least a 20%, 50%, 2-fold, or 5-fold increase in target mammalian cell killing as compared to the same amount of a control ABPC.

Embodiment 65. The ABPC of any one of embodiments 53-64, wherein the ABPC provides for an increase in endolysosomal delivery in the target mammalian cell as compared to the same amount of a control ABPC; optionally wherein the increase is at least a 20%, 50%, 2-fold, or 5-fold increase.

Embodiment 66. The ABPC of any one of embodiments 53-65, wherein the ABPC results in a less or no detectable reduction in the level of TROP2 presented on the surface of the target mammalian cell as compared to the same amount of a control ABPC.

Embodiment 67. The ABPC of any one of embodiments 53-66, wherein the ABPC further comprises a second ABD.

Embodiment 68. The ABPC of any one of embodiments 53-67, wherein the ABPC is conjugated to a drug.

Embodiment 69. A kit comprising at least one dose of the ABPC of any one of embodiments 53-68.

Embodiment 70. A method of treating a cancer characterized by having a population of cancer cells that have a predetermined level of TROP2 or an epitope of TROP2 presented on their surface, comprising administering a therapeutically effective amount of the composition of any one of embodiments 1-51 or the ABPC of any one of embodiments 53-68 to a subject identified as having a cancer characterized by having the population of cancer cells.

Embodiment 71. A method of reducing the volume of a tumor in a subject, wherein the tumor is characterized by having TROP2 positive cancer cells, comprising administering a therapeutically effective amount of the composition of any one of embodiments 1-51 or the ABPC of any one of embodiments 53-68 to the subject identified as having a cancer characterized by the cancer cells.

Embodiment 72. A method of inducing cell death in a TROP2 positive cancer cell in a subject, comprising administering a therapeutically effective amount of the composition of any one of embodiments 1-51 or the ABPC of any one of embodiments 53-68 to a subject identified as having the cancer.

Embodiment 73. A method of decreasing the risk of developing a metastasis or an additional metastasis in a subject having a TROP2 positive cancer, comprising administering a therapeutically effective amount of the composition of any one of embodiments 1-51 or the ABPC of any one of embodiments 53-68 to the subject identified as having the cancer.

Embodiment 74. A TROP2-specific-binding protein construct comprising a heavy chain variable domain and a light chain variable domain set forth in one of the following pairs of sequences, respectively: SEQ ID NOs: 18+59, 19+59, 32+59, 34+59, 35+59, 36+59, 37+59, 38+59, 18+62, 19+62, 38+62, 19+65, 18+66, 19+66, 35-71, 38+71, 34+73, 19+75, 35+75, 36+75, 37+75, 38+75, 19+366, 32+366, 35+366, 36+366, 18+376, 32+376, 34+376, 35+376, 36+376, 37+376, 38+376, 18+351, 19+351, 32+351, 34+351, 35+351, 36+351, 37+351, 38+351, 18+352, 19+352, 32+352, 34+352, 35+352, 36+352, 37+352, 38+352, 32+353, 34+353, 35+353, 37+353, 38+353, 18+361, 19+361, 32+361, 34+361, 35+361, 36+361, 37+361, 38+361, 18+365, 32+365, 34+365, 35+365, 36+365, 37+365, 38+365, 18+366, 19+366, 32+366, 34+366, 35+366, 36+366, 37+366, 38+366, 18+388, 32+388, 34+388, 35+388, 37+388, 38+388, 18+389, 19+389, 32+389, 34-389, 35+389, 36+389, 37+389, 38+389, 18+394, 32+394, 34+394, 35+394, 36+394, 37-394, and 38+394.

Embodiment 75. A TROP2-specific-binding protein construct comprising a heavy chain variable domain and a light chain variable domain set forth in one of the following pairs of sequences, respectively: SEQ ID NOs: 101+134, 119+134, 122+134, 119+142, 119+143, 122+143, 116+148, 116+151, 119+151, and 122+151.

Embodiment 76. A TROP2-specific-binding protein construct comprising a heavy chain variable domain and a light chain variable domain set forth in one of the following pairs of sequences, respectively: SEQ ID NOs: 196+223, 197+223, 200+223, 196+224, 197+224, 200+224, 196+225, 197+225, 200+225, 196+226, 197+226, 196+229, 197+229, 200+229, 196+233, 197+233, 197+460, 197+462, 197+460, or 197+462.

Embodiment 77. A TROP2-specific-binding protein construct comprising a heavy chain variable domain (HCVD) and a light chain variable domain (LCVD) comprising one of (a) to (d):
(a) one of the pairs of HCVD and LCVD set forth in one of the following pairs of SEQ ID NOs: 18+59, 19+59, 32+59, 34+59, 35+59, 36+59, 37+59, 38+59, 18+62, 19+62, 38+62, 19+65, 18+66, 19+66, 35+71, 38+71, 34+73, 19+75, 35+75, 36+75, 37+75, 38+75, 19+366, 32+366, 35-366, 36+366, 18+376, 32+376, 34+376, 35+376, 36+376, 37+376, 38+376, 18+351, 19-351, 32+351, 34+351, 35+351, 36+351, 37+351, 38+351, 18+352, 19+352, 32+352, 34+352, 35+352, 36+352, 37+352, 38+352, 32+353, 34+353, 35+353, 37+353, 38+353, 18+361, 19+361, 32+361, 34-361, 35+361, 36+361, 37+361, 38+361, 18+365, 32+365, 34+365, 35+365, 36+365, 37-365, 38+365, 18+366, 19+366, 32+366, 34+366, 35+366, 36+366, 37+366, 38+366, 18+388, 32+388, 34+388, 35+388, 37+388, 38+388, 18+389, 19+389, 32+389, 34+389, 35+389, 36+389, 37+389, 38+389, 18+394, 32+394, 34+394, 35+394, 36+394, 37+394, 38+394, 101-134, 119+134, 122+134, 119+142, 119+143, 122+143, 116+148, 116+151, 119+151, 122+151, 196+223, 197+223, 200+223, 196+224, 197+224, 200+224, 196+225, 197+225, 200+225, 196+226, 197+226, 196+229, 197+229, 200+229, 196+233, 197+233, 197+460, 197+462, 197+460, and 197+462; (b) an HCVD sequence and an LCVD sequence each having the same HCDRs and LCDRs of, and at least 90%, 95%, or 98% identity to, the HCVD and LCVD recited in one of the pairs of SEQ ID NOs: recited in (a); (c) an HCVD sequence and an LCVD sequence each having the same HCDRs and LCDRs of (as determined by the Kabat system), and at least 90%, 95%, or 98% identity to, the HCVD and LCVD recited in one of the pairs of SEQ ID NOs: recited in (a); and (d) an HCVD sequence and an LCVD sequence each having the same HCDRs and LCDRs of (as determined by the IMGT system), and at least 90%, 95%, or 98% identity to, the HCVD and LCVD recited in one of the pairs of SEQ ID NOs: recited in (a).

Embodiment 78. The antigen binding protein construct (ABPC) or composition of any one of the preceding embodiments for use in a method of treating a cancer characterized by having a population of cancer cells that have a predetermined level of TROP2 or an epitope of TROP2 presented on their surface.

Embodiment 79. The antigen binding protein construct (ABPC) or composition of any one of the preceding embodiments for use in a method of reducing the volume of a tumor in a subject, wherein the tumor is characterized by having TROP2 positive cancer cells.

Embodiment 80. The antigen binding protein construct (ABPC) or composition of any one of the preceding embodiments for use in a method of inducing cell death in a TROP2 positive cancer cell in a subject.

Embodiment 81. The antigen binding protein construct (ABPC) or composition of any one of the preceding embodiments for use in a method of decreasing the risk of developing a metastasis or an additional metastasis in a subject having a TROP2 positive cancer.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of this following claims.

What is claimed is:

1. An antigen-binding protein construct (ABPC) comprising: a first antigen-binding domain (ABD) that is capable of specifically binding TROP2 or an epitope of TROP2 presented on the surface of a target mammalian cell, wherein: the dissociation rate of the first ABD at a pH of ~4.0-6.5 is faster than the dissociation rate at a pH of ~7.0-8.0; or the K_{D} of the first ABD at a pH of ~4.0-6.5 is greater than the K_{D} at a pH of ~7.0-8.0.

2. The ABPC of claim 1, wherein the first ABD comprises a heavy chain variable domain (HCVD) of one of an HCVD of (a) sacituzumab; (b) datopotamab; (c) hu7E6SVG, and (d) A1,2X4 and/or a light chain variable domain (LCVD) of one of an LCVD of (a) sacituzumab; (b) datopotamab; (c) hu7E6SVG; and (d) A1,2X4, each HCVD or LCVD with one or more amino acids substituted with a histidine.

3. The ABPC of claim 2, wherein the HCVD comprises one of: (a1) an HCVD of sacituzumab comprising SEQ ID NO: 4; (b1) an HCVD of datopotamab comprising SEQ ID NO: 80; (c1) an HCVD of hu7E6SVG comprising SEQ ID NO: 156; and (d1) an HCVD of A1,2X4 comprising SEQ ID NO: 234; and one of a corresponding: (a2) an LCVD of sacituzumab comprising SEQ ID NO: 5; (b2) an LCVD of datopotamab comprising SEQ ID NO: 81; (c2) an LCVD of hu7E6SVG comprising SEQ ID NO: 157; and (d2) an LCVD of A1,2X4 comprising SEQ ID NO: 235, wherein each HCVD and/or LCVD has one or more histidine substitution.

4. The ABPC of claim 3, wherein the first ABD comprises an HCVD comprising the CDR1, 2, and 3 of one of (a) to (c): (a1) SEQ ID NOs: 6, 7, and 8; (b1) SEQ ID NOs: 82, 83, and 84; (c1) SEQ ID NOs: 158, 159, and 160; and (d1) SEQ ID NOs: 236, 237, and 238, and a corresponding LCVD comprising the CDR1, 2, and 3 of one of a corresponding: (a2) SEQ ID NOs: 9, 10, and 11; (b2) SEQ ID NOs: 85, 86, and 87; (c2) SEQ ID NOs: 161, 162, and 163; and (d2) SEQ ID NOs: 239, 240, and 241, wherein each set of three CDRs has collectively one or more amino acid position substituted with a histidine.

5. The ABPC of any one of claims 2-4, comprising an HCVD of one of (a) to (c):
(a) an HCVD that is at least 90%, 95%, or 98% identical to SEQ ID NO: 4, wherein the HCVD includes a histidine at one or more positions in SEQ ID NO: 4 selected from 29, 32, 33, 50, 52, 53, 60, 61, 62, 63, 64, 65, 66, 99, 101, 104, 105, and 108;
(b) an HCVD that is at least 90%, 95%, or 98% identical to SEQ ID NO: 80, wherein the HCVD includes a histidine at one or more positions in SEQ ID NO: 80 selected from 29, 53, 55, 64, 65, 98, 101, 104, 105, 107, and 108; and
(c) an HCVD that is at least 90%, 95%, or 98% identical to SEQ ID NO: 156, wherein the HCVD includes a histidine at one or more positions in SEQ ID NO: 156 selected from 26, 29, 30, 33, 34, 51, 54, 56, 57, 59, 102, 103, and 106.

6. The ABPC of any one of claims 2-5, comprising an LCVD of one of (a) to (d):
(a) an LCVD that is at least 90%, 95%, or 98% identical to SEQ ID NO: 5, wherein the LCVD includes a histidine at one or more positions in SEQ ID NO: 5 selected from 28, 29, 30, 31, 32, 33, 50, 51, 52, 53, 89, 92, 93, 94, 95, 96, and 97;
(b) an LCVD that is at least 90%, 95%, or 98% identical to SEQ ID NO: 80, wherein the LCVD includes a histidine at one or more positions in SEQ ID NO: 80 selected from 26, 28-31, 33, 34, 50-54, 89, 93, 94, and 95;
(c) an LCVD that is at least 90%, 95%, or 98% identical to SEQ ID NO: 157, wherein the LCVD includes a histidine at one or more positions in SEQ ID NO: 157 selected from 25, 32, 34, 36, 54, 56, 57, 58, 59, 60, 93, 96, 97, 99, 100, and 101; and
(d) an LCVD that is at least 90%, 95%, or 98% identical to SEQ ID NO: 235, wherein the LCVD includes a histidine at one or more positions in SEQ ID NO: 235.

7. The ABPC of 5 or 6, wherein the HCVD and/or LCVD includes a histidine at two or more positions in its defining SEQ ID NO.

8. The ABPC of claim 1, wherein the first ABD comprises an HCVD comprising the sequence of SEQ ID NO: 4, one of the sequences of SEQ ID NOs: 12-52, SEQ ID NO: 80, one of the sequences of SEQ ID NOs: 88-128, SEQ ID NO: 156, one of the sequences of 164-202, SEQ ID NO: 234, one of the sequences set forth in SEQ ID NOs: 242-280, or one of the sequences set forth in SEQ ID NOs: 318, 320, 322, 323, 324, 325, 328, 332, 333, 334, 336, 337, 343, 344, 345, 347, 348, 427, 428, 429, 430, 431, 433, 434, 435, 436, 437, 438, 439, 440, 441, 443, 444, 445, 446, 447, 449, 451, 454, 456, and 457; and/or the first ABD comprises an LCVD comprising the sequence of SEQ ID NO: 5, one of the sequences set forth in SEQ ID NOs: 53-79, SEQ ID NO: 81, one of the sequences set forth in SEQ ID NOs: 129-155, SEQ ID NO: 157, one of the sequences set forth in SEQ ID NOs: 203-233, SEQ ID NO: 235, one of the sequences set forth in SEQ ID NOs: 281-312, or one of the sequences set forth in SEQ ID NOs: 351, 352, 353, 354, 355, 359, 360, 361, 362, 365, 366, 367, 368, 370, 371, 372, 373, 376, 377, 383, 386, 387, 388, 389, 392, 393, 394, 395, 396, 411, 412, 417, 418, 423, 459, 460, 462, 464, 465, 466, 467, 468, 469, 470, 473, 474, 485, 488, 489, and 491.

9. The ABPC of claim 1, wherein the first ABD comprises one of (a) to (c):
(a) an HCVD of SEQ ID NO: 4, one of the sequences set forth in SEQ ID NOs: 12-52, or one of the sequences set forth in SEQ ID NOs: 313-350, and/or an LCVD of SEQ ID NO: 5, one of the sequences set forth in SEQ ID NOs: 53-79, or one of the sequences set forth in SEQ ID NOs: 351-396, wherein the first ABD does not comprise (i) SEQ ID NOs: 4 and 5; (ii) an HCVD of SEQ ID NO: 4 and an LCVD that is not one of the sequences set forth in SEQ ID NOs: 57, 58, 59, 60, 61, 62, 64, 65, 66, 67, 71, 74, 75, 76, 77, 78, 79, 351, 352, 353, 354, 355, 359, 360, 361, 362, 365, 366, 367, 368, 370, 371, 372, 373, 376, 377, 383, 386, 387, 388, 389, 392, 393, 394, 395, and 396; or (iii) an LCVD of SEQ ID NO: 5 and an HCVD that is not one of SEQ ID NOs: 15, 18, 19, 22, 24, 25, 32, 33, 34, 35, 36, 37, 38, 41, 43, 46, 47, 50, 318, 320, 322, 323, 324, 325, 328, 332, 333, 334, 336, 337, 343, 344, 345, 347, and 348;
(b) an HCVD of SEQ ID NO: 80, one of the sequences set forth in SEQ ID NOs: 88-128, one of the sequences set forth in SEQ ID NOs: 397-406, or one of the sequences set forth in SEQ ID NOs: 397-406, and/or an LCVD of SEQ ID NO: 81, one of the sequences set forth in SEQ ID NOs: 129-155, or one of the sequences set forth in SEQ ID NOs: 407-426, wherein the first ABD does not comprise (i) SEQ ID NOs: 80 and 81; (ii) an HCVD of SEQ ID NO: 80 and an LCVD that is not one of SEQ ID NOs: 131, 133, 134, 135, 136, 138, 139, 140, 141, 142, 143, 144, 147, 151, 152, 153, 411, 412, 417, 418, and 423; or (iii) an LCVD of SEQ ID NO: 81 and an HCVD that is not one of SEQ ID NOs: 91, 101, 103, 112, 113, 116, 119, 122, 123, 125, and 126; and
(c) an HCVD of SEQ ID NO: 156, one of the sequences set forth in SEQ ID NOs: 164-202, or one of the sequences set forth in SEQ ID NOs: 427-457, and/or an LCVD of SEQ ID NO: 157, one of the sequences set forth in SEQ ID NOs: 203-233, or one of the sequences set forth in SEQ ID NOs: 458-492, wherein the first ABD does not comprise (i) SEQ ID NOs: 156 and 157; (ii) an HCVD of SEQ ID NO: 156 and an LCVD that is not one of SEQ ID NOs: 204, 211, 213, 215, 218, 220, 221, 222, 223, 224, 225, 228, 229, 231, 232, 233, 459, 460, 462, 464, 465, 466, 467, 468, 469, 470, 473, 474, 485, 488, 489, and 491; or (iii) an LCVD of SEQ ID NO: 157 and an HCVD that is not one of the sequences set forth in SEQ ID NOs: 164, 167, 168, 171, 172, 175, 178, 180, 181, 183, 196, 197, 200, 201, 427, 428, 429, 430, 431, 433, 434, 435, 436, 437, 438, 439, 440, 441, 443, 444, 445, 446, 447, 449, 451, 454, 456, and 457.

10. The ABPC of any one of claims 1-9, wherein the ABPC is degraded in the target mammalian cell following internalization of the ABPC by the target mammalian cell; and/or wherein the ABPC further comprises a conjugated toxin, radioisotope, drug, or small molecule; and/or wherein the ABPC provides for an increase in toxin liberation in the target mammalian cell as compared to the same amount of a control ABPC.

11. The ABPC of claim 10, wherein the ABPC provides for at least a 20%, 50%, 2-fold, or 5-fold increase in toxin liberation; and/or wherein the ABPC provides for an increase in target mammalian cell killing as compared to the same amount of a control ABPC.

12. The ABPC of claim 11, wherein the composition provides for at least a 20%, 50%, 2-fold, or 5-fold increase in target mammalian cell killing as compared to the same amount of a control ABPC.

13. The ABPC of any one of claims 1-12, wherein the ABPC provides for an increase in endolysosomal delivery in the target mammalian cell as compared to the same amount of a control ABPC; optionally wherein the increase is at least a 20%, 50%, 2-fold, or 5-fold increase.

14. The ABPC of any one of claims 1-13, wherein the ABPC results in a less or no detectable reduction in the level of TROP2 presented on the surface of the target mammalian cell as compared to the same amount of a control ABPC.

15. The ABPC of any one of claims 1-14, wherein the ABPC further comprises a second ABD.

16. The ABPC of any one of claims 1-15, wherein the ABPC is conjugated to a drug.

17. A kit comprising at least one dose of the ABPC of any one of claims 1-16.

18. A method of treating a cancer characterized by having a population of cancer cells that have a predetermined level of TROP2 or an epitope of TROP2 presented on their surface, comprising administering a therapeutically effective amount of the ABPC of any one of claims 1-16 to a subject identified as having a cancer characterized by having the population of cancer cells.

19. A method of reducing the volume of a tumor in a subject, wherein the tumor is characterized by having TROP2 positive cancer cells, comprising administering a therapeutically effective amount of the ABPC of any one of claims 1-16 to the subject identified as having a cancer characterized by the cancer cells.

20. A method of inducing cell death in a TROP2 positive cancer cell in a subject, comprising administering a therapeutically effective amount of the ABPC of any one of claims 1-16 to the subject identified as having the cancer.

21. A method of decreasing the risk of developing a metastasis or an additional metastasis in a subject having a TROP2 positive cancer, comprising administering a therapeutically effective amount of the ABPC of any one of claims 1-16 to the subject identified as having the cancer.

22. A TROP2-specific-binding protein construct comprising a heavy chain variable domain (HCVD) and a light chain variable domain (LCVD) comprising one of (a) to (f):
(a) one of the pairs of HCVD and LCVD sequences set forth in one of the following pairs of SEQ ID NOs: 18+59, 19+59, 32+59, 34+59, 35+59, 36+59, 37+59, 38+59, 18+62, 19+62, 38+62, 19+65, 18+66, 19+66, 35+71, 38+71, 34+73, 19+75, 35+75, 36+75, 37+75, 38+75, 19+366, 32+366, 35+366, 36+366, 18+376, 32+376, 34+376, 35+376, 36+376, 37+376, 38+376, 18+351, 19+351, 32+351, 34+351, 35+351, 36+351, 37+351, 38+351, 18+352, 19+352, 32+352, 34+352, 35+352, 36+352, 37+352, 38+352, 32+353, 34+353, 35+353, 37+353, 38+353, 18+361, 19+361, 32+361, 34+361, 35+361, 36+361, 37+361, 38+361, 18+365, 32+365, 34+365, 35+365, 36+365, 37+365, 38+365, 18+366, 19+366, 32+366, 34+366, 35+366, 36+366, 37+366, 38+366, 18+388, 32+388, 34+388, 35+388, 37+388, 38+388, 18+389, 19+389, 32+389, 34+389, 35+389, 36+389, 37+389, 38+389, 18+394, 32+394, 34+394, 35+394, 36+394, 37+394, 38+394, 101+134, 119+134, 122+134, 119+142, 119+143, 122+143, 116+148, 116+151, 119+151, 122+151, 196+223, 197+223, 200+223, 196+224, 197+224, 200+224, 196+225, 197+225, 200+225, 196+226, 197+226, 196+229, 197+229, 200+229, 196+233, 197+233, 197+460, 197+462, 197+460, and 197+462;
(b) an HCVD sequence and an LCVD sequence each having the same HCDRs and LCDRs of, and at least 90%, at least 95%, or at least 98% identity to the HCVD and LCVD sequences recited in one of the pairs of SEQ ID NOs: recited in (a);
(c) an HCVD sequence and an LCVD sequence each having the same HCDRs and LCDRs (as determined by the Kabat system) of, and at least 90%, at least 95%, or at least 98% identity to, the HCVD and LCVD sequences set forth in one of the pairs of SEQ ID NOs: recited in (a);
(d) an HCVD sequence and an LCVD sequence each having the same HCDRs and LCDRs (as determined by the IMGT system) of, and at least 90%, at least 95%, or at least 98% identity to, the HCVD and LCVD sequences set forth in one of the pairs of SEQ ID NOs: recited in (a);
(e) an HCVD sequence and an LCVD sequence each having the same HCDRs and LCDRs (as determined by the additive Kabat-IMGT method) of, and at least 90%, at least 95%, or at least 98% identity to, the HCVD and LCVD sequences set forth in one of the pairs of SEQ ID NOs: recited in (a); and
(f) an HCVD sequence and an LCVD sequence each having the same HCDRs and LCDRs (as determined by the additive Kabat-IMGT method) of, and at least 98% identity to, the HCVD and LCVD sequences set forth in one of the pairs of SEQ ID NOs: recited in (a).
